(19) United States
(12) Patent Application Publication
(10) Pub. No.: US 2025/0049908 A1
(43) Pub. Date: Feb. 13, 2025

(54) HIV VACCINES AND METHODS OF USING

(71) Applicant: Gilead Sciences, Inc., Foster City, CA (US)

(72) Inventors: Jiani Li, San Francisco, CA (US); Xinan Liu, Shanghai (CN); Azure T. Makadzange, Half Moon Bay, CA (US); Stephen R. Martin, Tiburon, CA (US); Sarah Schmidt, Vienna (AT); Hesham Shehata, Sunnyvale, CA (US); Felix Stemeseder, Vienna (AT); Evguenia Svarovskaia, Redwood City, CA (US); Archana V. Boopathy, San Mateo, CA (US)

(21) Appl. No.: 18/743,701

(22) Filed: Jun. 14, 2024

Related U.S. Application Data
(63) Continuation of application No. 17/574,465, filed on Jan. 12, 2022, now Pat. No. 12,053,517.
(60) Provisional application No. 63/170,900, filed on Apr. 5, 2021.
(60) Provisional application No. 63/149,820, filed on Feb. 16, 2021.
(60) Provisional application No. 63/137,521, filed on Jan. 14, 2021.

(51) Int. Cl. A61K 39/12; A61K 39/00; A61P 31/18; C07K 14/005; C07K 16/10; C07K 16/28; C12N 15/10; C12N 15/86

(52) U.S. Cl. CPC A61K 39/12; A61P 31/18; C07K 14/005; C07K 16/1045; C07K 16/2818; C07K 16/2827; C12N 15/1082; C12N 15/86; A61K 2039/53; A61K 2039/70; C12N 2710/10343; C12N 2710/16143; C12N 2740/15034; C12N 2740/16022; C12N 2740/16034; C12N 2740/16134; C12N 2740/16234; C12N 2760/10043

(57) ABSTRACT

Provided are HIV-1 fusion polypeptides, polynucleotides encoding such fusion polypeptides, vectors expressing such fusion polypeptides for use in eliciting an immune response against HIV-1; pharmaceutical and immunogenic compositions and kits comprising such fusion polypeptides, polynucleotides or vectors, and methods of use in treating and/or preventing HIV-1. Further provided are methods for design of antiviral vaccines, including vaccines to elicit an immune response against HIV-1.

### CROSS-REFERENCE TO RELATED APPLICATIONS

This application is a Continuation of U.S. application Ser. No. 17/574,465, filed on Jan. 12, 2022, and granted as U.S. Pat. No. 12,053,517 on Aug. 6, 2024, which claims the benefit under 35 U.S.C. § 119(e) of U.S. Provisional Application No. 63/137,521, filed on Jan. 14, 2021; U.S. Provisional Application No. 63/149,820, filed on Feb. 16, 2021 and U.S. Provisional Application No. 63/170,900, filed on Apr. 5, 2021, which are hereby incorporated herein by reference in their entireties for all purposes.

### SEQUENCE LISTING

The instant application contains a Sequence Listing which has been submitted electronically in XML format and is hereby incorporated by reference in its entirety. Said XML copy, created on Aug. 7, 2024 is named 1355-US-NP.xml and is 379,651 bytes in size.

### BACKGROUND

HIV remains one of the leading causes of mobility and mortality globally, with over 38 million infected globally and 690,000 deaths in 2019 (UNAIDS. Fact Sheet-World AIDS Day 2020. unaidsorg/en/resources/fact-sheet. 2020). The last three decades have seen significant improvements in therapeutics for HIV. The development of highly active anti-retroviral therapy has improved survival for people living with HIV, and reduced morbidity from HIV related immunosuppression and opportunistic infections (Johnson, et al., PLOS Med. 2013; 10 (4): e1001418; May, et al., AIDS. (2014) 28 (8): 1193-202; Rodger, et al., AIDS. (2013) 27 (6): 973-9; Samji, et al., PLOS One. (2013) 8 (12): e81355). In recent years antiretroviral treatment (ART) regimens have been simplified to once daily pills with longer acting oral and injectable therapeutics on the horizon (Swindells, et al., N Engl J Med. (2020) 382 (12): 1112-23; Orkin, et al., N Engl J Med. (2020) 382 (12): 1124-35; Link, et al., Nature. (2020) 584 (7822): 614-8). Despite these advances in therapeutics, to date there has only been two well documented examples of cure, both of which required severe immunosuppression and bone marrow transplantation (Gupta, et al., Lancet HIV. (2020) 7 (5): e340-e7; Hutter, et al., N Engl J Med. (2009) 360 (7): 692-8). Unlike many self-limited infectious diseases, the HIV virus integrates itself into its host genome, and establishes latency in resting memory CD4+T cells (Eisele, et al., Immunity. (2012) 37 (3): 377-88). These cells form the HIV latent reservoir that cannot be cleared by standard antiretroviral therapy. Reservoir cells are also protected from immune surveillance and clearance mechanisms, as they do not express viral antigens enabling them to evade recognition and clearance by cytotoxic T cells. The reservoir is long-lived and despite detectable reductions in the size of the reservoir, viral rebound is observed following discontinuation of ART (Henrich, et al., Ann Intern Med. (2014) 161 (5): 319-27; Henrich, et al., J Infect Dis. (2013) 207 (11): 1694-702). Activating the reservoir with latency reversing agents will need to be coupled with an effective mechanism to stimulate cytotoxic T cells and eliminate the infected reservoir cells (Ait-Ammar, et al., Front Microbiol. (2019) 10:3060; Mothe, et al., Front Immunol. (2020) 11:823; Fidler, et al., Lancet. (2020) 395 (10227): 888-98). A therapeutic vaccine designed to generate antigen specific effector T cell responses can be an additional component of an HIV cure strategy.

T cell vaccines hold significant promise in therapeutic areas such as oncology. High levels of tumor infiltrating CD8+ lymphocytes are associated with better prognosis in some cancers, leading to significant interest in developing vaccines that can induce tumor specific cytotoxic CD8+T cells. Although previous approaches have had limited success (Rosenberg, et al., Nature Medicine. (2004) 10 (9): 909-15), the use of novel delivery platforms, improved techniques in antigen discovery and immune modulation hold some promise (Ott, et al., Nature. (2017) 547 (7662): 217-21). Similarly, T cells play an important role in the control of HIV viremia. Antigen specific T cells that arise in acute HIV infection are responsible for driving the initial drop in viremia (Borrow, et al., J Virol. (1994) 68 (9): 6103-10; Koup, et al., J Virol. (1994) 68 (7): 4650-5). HIV infected human long-term non-progressers or elite controllers are characterized by having strong effective antigen specific T cell responses. In SIV infection (a non-human primate model of human HIV infection) antigen specific T cells either generated naturally or through vaccination have been associated with viral control (Schmitz, et al., Science. (1999) 283 (5403): 857-60; Jin, et al., J Exp Med. (1999) 189 (6): 991-8). Despite this data, T-cells vaccines for prevention have had limited success in inducing T cell responses of limited breadth or efficacy (Buchbinder, et al., Lancet. (2008) 372 (9653): 1881-93; Janes, et al., J Infect Dis. (2013) 208 (8): 1231-9; Excler, et al., Curr Opin HIV AIDS. (2016) 11 (6): 607-13). Therapeutic vaccine trials with T cell vaccines have also shown limited efficacy (Mothe, et al., Front Immunol. (2020) 11:823; Colby, et al., Nature Medicine. (2020) 26 (4): 498-501). These trials have used viral vectors such as adenoviruses (human and chimpanzee) as well as modified vaccinia Ankara (MVA) virus and with full length viral antigens or shorter constructs (Barouch, et al., Lancet. (2018) 392 (10143): 232-43; Mothe, et al., EClinicalMedicine. (2019) 11:65-80). Data from these studies suggest that following vaccination the breadth of antigen specific T cells generated is low. Although these vaccine-induced T cells secrete IFN-γ in standard ELISpot assays the demonstrated lack of efficacy suggests that these T cells may not have full cytotoxic activity.

Immunogen design is a component of any therapeutic HIV vaccine to generate the right antigen specific response, targeted at a conserved region and with cytotoxic activity. Natural infection has demonstrated that the immune response will tend to focus on highly immunodominant variable regions within HIV for example in HIV envelope or Nef, or variable regions in HIV Gag (Addo, et al. J Virol. (2003) 77 (3): 2081-92; Betts, et al., J Virol. 2001; 75 (24): 11983-91). These responses generate T cells from which the virus can rapidly escape without a fitness cost (Liu, et al., J Virol. (2006) 80 (19): 9519-29). An effective HIV vaccine will need to avoid these regions and drive the establishment of de novo responses that focus the immune response to conserved regions of the virus where conservation is required to maintain viral function.

Previous HIV vaccines have primarily focused on designing immunogens that provide universal coverage by addressing global HIV viral diversity and have generated full length sequences or have adapted algorithms to generate constructs of conserved regions (Korber, et al., Hum Vaccin Immunother. (2020) 16 (3): 713-722; Fischer, et al., Nature Medicine. (2007) 13 (1): 100-6). All of these approaches have primarily evaluated consensus sequences assessing inter patient variability to determine regions of conservation, and concatenated to provide global sequence coverage. However, within an HIV infected individual there are several circulating quasispecies. Some of these viral quasispecies are generated as a result of T cell driven pressure and reflect pre-existing escape route for the virus should a T cell response directed at it emerge (Liu, et al., J Virol. (2006) 80 (19): 9519-29; Korber, et al., 2020, supra; Price, et al., Proc Natl Acad Sci USA. (1997) 94 (5): 1890-5). That the targeting of conserved regions by previous vaccines did not consider quasispecies together with identification of highly conserved immunogenic regions, may in part explain their limited efficacy (Hanke, et al., Expert Rev Vaccines. (2019) 18 (10): 1029-41).

### SUMMARY

Provided are fusion polypeptides comprising a plurality of polypeptide segments of one or more human immunodeficiency virus-1 (HIV-1) proteins encoded by two or more HIV genes selected from gag, pol and nef. In some embodiments, the plurality of polypeptide segments comprises or consists of polypeptide segments encoded by HIV-1 genes pol and nef, and does not comprise polypeptide segments encoded by HIV-1 genes env, tat, rev, vpr, vif and/or vpu. In some embodiments, the plurality of polypeptide segments comprises or consists of polypeptide segments encoded by HIV-1 genes gag, pol and nef, and does not comprise polypeptide segments encoded by HIV-1 genes env, tat, rev, vpr, vif and/or vpu. In some embodiments, the plurality polypeptide segments are derived from conserved regions in a population of viral proteome sequences. In some embodiments, the conserved regions are greater than 80%, 81%, 82%, 83%, 84%, 85%, 86%, 87%, 88%, 89%, 90%, 91%, 92%, 93%, 94%, 95%, 96%, 97%, 98% or 99% conserved amongst HIV-1 species in interpatient populations. In some embodiments, the conserved regions are conserved amongst one or more of HIV-1 clades A-K, e.g., one or more of clades A, B, C, D and G, or recombinant forms of one or more of HIV-1 clades A-K, and combinations thereof. In some embodiments, the fusion polypeptides comprise at least 4 and up to 6 polypeptide segments, e.g., 4, 5 or 6 polypeptide segments. In some embodiments, each polypeptide segment is at least 10 amino acids in length, and up to about 225 amino acids in length, e.g. from at least 10 amino acids in length up to 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220 or 225 amino acids in length. In some embodiments, the full-length of the fusion polypeptide comprises at least about 330 amino acids and up to about 505 amino acids (without a signal peptide) or up to about 550 amino acids (including a signal peptide), e.g., at least about 330 amino acids and up to about 335, 340, 345, 350, 355, 360, 365, 370, 375, 380, 385, 390, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545 or 550 amino acids. In some embodiments, the full-length of the fusion polypeptide is no longer than 550 amino acids (with a signal peptide) or no longer than 505 amino acids (without a signal peptide), e.g., no longer than 545, 540, 535, 530, 525, 520, 515, 510, 505, 500, 495, 490, 485, 480, 475, 470, 465, 460, 455, 450, 445, 440, 435, 430, 425, 420, 415, 410, 405, 400, 390, 385, 380, 375, 370, 365, 360, 355, 350, 345, 340, 335 or 330 amino acids. In some embodiments, the full-length of the fusion polypeptide comprises at least 350 amino acids and up to 385 amino acids, e.g., at least 350 amino acids and up to 365 amino acids. In some embodiments, the full-length of the fusion polypeptide comprises at least 390 amino acids and up to 395 amino acids. In some embodiments, each polypeptide segment comprises or consists of one or more predicted T cell epitopes. In some embodiments, the fusion polypeptide comprises three or more predicted T cell epitopes, e.g., four, five, six or more predicted T cell epitopes. In some embodiments, the fusion polypeptide comprises one or more polypeptide segments that bind to or are presented by one or more human HLA class I alleles (e.g. 1, 2, 3, 4, 5 or 6 alleles), e.g. within a single subject or amongst multiple patients. In some embodiments, the fusion polypeptide comprises one or more polypeptide segments that bind to or are presented by at least by a human A*0201 HLA class I molecule. In some embodiments, the fusion polypeptide comprises one or more polypeptide segments that are intracellularly processed and presented by one or more human HLA class II alleles (e.g. 1, 2, 3, 4, 5 or 6 alleles), e.g. within a single subject. In some embodiments, one or more of the polypeptide segments is abutted or fused to an adjacent segment. In some embodiments, one or more of the polypeptide segments is joined to an adjacent segment by one or more peptide linkers. In some embodiments, the one or more peptide linkers is selected from one or more of a polyalanine, a polyglycine, Gln-Glu-Glu (QEE), Lysine (L), Isoleucine (I), Leu-Ile (LI), Lys-Ile-Leu (KIL), Leu-Ile-Lys (LIK), Pro-Pro-Val (PPV), Ser-Glu-Gly (SEG), a cleavable linker, a flexible linker, a rigid linker, and combinations thereof. In some embodiments, the flexible linker is selected from Gln-Glu-Glu (QEE) (SEQ ID NO: 51), Lysine (L), Isoleucine (I), Leu-Ile (LI), Lys-Ile-Leu (KIL) (SEQ ID NO: 52), Leu-Ile-Lys (LIK) (SEQ ID NO: 53), Pro-Pro-Val (PPV) (SEQ ID NO: 54) and Ser-Glu-Gly (SEG) (SEQ ID NO:55). In some embodiments, the flexible linker comprises a polyalanine linker or a polyglycine linker, e.g., comprising or consisting of 2 or 3 contiguous alanine residues, e.g. AA, AAA (SEQ ID NO: 48), AAY (SEQ ID NO: 49) or AAX, wherein X is any amino acid (e.g. A, C, D, E, F, G, H, I, K, L, M, N, P, Q, R, S, T, V, Y) (SEQ ID NO: 50). In some embodiments, the flexible linker comprises or consists of GG, GGS (SEQ ID NO: 57), GSG (SEQ ID NO: 58) or GGGS (SEQ ID NO: 59). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. RAKR (SEQ ID NO: 60), REKR (SEQ ID NO: 61) and RRKR (SEQ ID NO: 62)), and combinations, derivatives or variants thereof. In some embodiments, the cleavable linker comprises or consists of a furin recognition/cleavage site selected from RAKR (SEQ ID NO: 60), REKR (SEQ ID NO: 61) and RRKR (SEQ ID NO: 62). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67), or comprises or consists of the amino acid sequence of ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67). In some embodiments, the fusion polypeptide comprises two, three, four, five, six, or more, of the polypeptide segments comprising or consisting of amino acid residues corresponding to Gag 1-53; Gag 147-369; Pol 56-117; Pol 129-320; Pol 367-431 Pol 542-606; Pol 586-606; Pol 683-708, Pol 747-827; Pol 840-909; Pol 840-920; Pol 932-1003; Nef 64-76; Nef 64-99 or Nef 117-148, wherein the Gag, Pol and Nef amino acid position numbers correspond to SEQ ID NOs: 1, 2 and 3, respectively. In some embodiments, the fusion polypeptide does not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In some embodiments, the fusion polypeptide does not comprise any polypeptide segments having an amino acid sequence of any one of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof. In some embodiments, the fusion polypeptide comprises two, three, four, five, six, or more, polypeptide segments selected from SEQ ID NOs: 4-33. In some embodiments, the fusion polypeptide comprises or consists of the following polypeptide segments, wherein the Gag, Pol and Nef amino acid position numbers correspond to SEQ ID NOs: 1, 2 and 3, respectively: (a) amino acid residues corresponding to: Gag 1-53; Gag 147-369; Pol 683-708 and Nef 117-148; (b) amino acid residues corresponding to: Pol 56-117; Pol 129-320; Pol 367-431 and Nef 64-99; (c) amino acid residues corresponding to: Pol 542-606; Pol 747-827; Pol 840-920; Pol 932-1003 and Nef 64-99; (d) amino acid residues corresponding to: Gag 1-53; Gag 147-369; Pol 683-708; Pol 747-827; Pol 840-920 and Nef 117-148; (e) amino acid residues corresponding to: Pol 56-117; Pol 129-320; Pol 367-431; Pol 542-606; Pol 932-1003 and Nef 64-99; (f) amino acid residues corresponding to: Gag 147-369, Pol 586-606, Pol 683-708 and Pol 840-920; (g) amino acid residues corresponding to: Pol 129-320, Pol 747-827, Pol 932-1003 and Nef 64-76; or (h) amino acid residues corresponding to: Gag: 147-369, Pol 747-827, Pol 840-909 and Nef 64-76. In some embodiments, the fusion polypeptide comprises or consists of the following polypeptide segments: (a) SEQ ID NOs: 4, 6, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 4, 6, 18 and 32, respectively; (b) SEQ ID NOs: 5, 7, 19 and 33, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 5, 7, 19 and 33, respectively; (c) SEQ ID NOs: 8, 10, 12 and 30 or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 10, 12 and 30, respectively; (d) SEQ ID NOs: 9, 11, 13 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 11, 13 and 31, respectively; (e) SEQ ID NOs: 14, 20, 24, 26 and 30, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 14, 20, 24, 26 and 30, respectively; (f) SEQ ID NOs: 15, 21, 25, 27 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 21, 25, 27 and 31, respectively; (g) SEQ ID NOs: 4, 6, 18, 20, 24 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 4, 6, 18, 20, 24 and 32, respectively; (h) SEQ ID NOs: 5, 7, 19, 21, 25 and 33, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 5, 7, 19, 21, 25 and 33, respectively; (i) SEQ ID NOs: 8, 10, 12, 14, 26 and 30, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 10, 12, 14, 26 and 30, respectively; (j) SEQ ID NOs: 9, 11, 13, 15, 27 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 11, 13, 15, 27 and 31, respectively; (k) SEQ ID NOs: 6, 16, 18 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 16, 18 and 24, respectively; (1) SEQ ID NOs: 7, 17, 19 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 17, 19 and 25, respectively; (m) SEQ ID NOs: 10, 20, 26 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 10, 20, 26 and 28, respectively; (n) SEQ ID NOs: 11, 21, 27 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 21, 27 and 29, respectively; (o) SEQ ID NOs: 6, 20, 22 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 22 and 28, respectively; (p) SEQ ID NOs: 7, 21, 23 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 23 and 29, respectively; (q) SEQ ID NOs: 6, 16, 18 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 16, 18 and 24, respectively; (r) SEQ ID NOs: 7, 17, 19 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 17, 19 and 25, respectively; (s) SEQ ID NOs: 10, 20, 26 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 10, 20, 26 and 28, respectively; or (t) SEQ ID NOs: 11, 21, 27 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 21, 27 and 29, respectively. In some embodiments, the fusion polypeptide comprises or consists of the following polypeptide segments in sequential order, from N-terminus to C-terminus, optionally joined or connected by one or more linkers: SEQ ID NOs: 6, 4, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 4, 18 and 32, respectively; SEQ ID NOs: 7, 5, 33 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 5, 33 and 19, respectively; SEQ ID NOs: 12, 30, 8 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 12, 30, 8 and 10, respectively; SEQ ID NOs: 9, 31, 13 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 31, 13 and 11, respectively; SEQ ID NOs: 14, 26, 20, 30 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 14, 26, 20, 30 and 24, respectively; SEQ ID NOs: 15, 31, 21, 27 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 31, 21, 27 and 25, respectively; SEQ ID NOs: 32, 18, 4 and 6, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 32, 18, 4 and 6, respectively; SEQ ID NOs: 7, 33, 5 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 33, 5 and 19, respectively; SEQ ID NOs: 8, 30, 12 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 30, 12 and 10, respectively; SEQ ID NOs: 13, 31, 9 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 13, 31, 9 and 11, respectively; SEQ ID NOs: 26, 30, 14, 20 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 30, 14, 20 and 24, respectively; SEQ ID NOs: 31, 27, 15, 25 and 21, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 31, 27, 15, 25 and 21, respectively; SEQ ID NOs: 24, 6, 4, 20, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 4, 20, 18 and 32, respectively; SEQ ID NOs: 6, 20, 4, 24, 32 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 4, 24, 32 and 18, respectively; SEQ ID NOs: 7, 21, 5, 25, 33 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 5, 25, 33 and 19, respectively; SEQ ID NOs: 8, 30, 14, 12, 26 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 30, 14, 12, 26 and 10, respectively; SEQ ID NOs: 8, 12, 30, 26, 14 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 12, 30, 26, 14 and 10, respectively; SEQ ID NOs: 9, 13, 31, 27, 15 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 13, 31, 27, 15 and 11, respectively; SEQ ID NOs: 20, 32, 24, 4, 6 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 20, 32, 24, 4, 6 and 18, respectively; SEQ ID NOs: 7, 25, 19, 5, 33 and 21, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 25, 19, 5, 33 and 21, respectively; SEQ ID NOs: 26, 30, 12, 14, 8 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 30, 12, 14, 8 and 10, respectively; SEQ ID NOs: 15, 31, 9, 27, 13 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 31, 9, 27, 13 and 11, respectively; SEQ ID NOs: 24, 6, 16 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 16 and 18, respectively; SEQ ID NOs: 7, 19, 17 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 19, 17 and 25, respectively; SEQ ID NOs: 24, 16, 6 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 16, 6 and 18, respectively; SEQ ID NOs: 7, 25, 17 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 25, 17 and 19, respectively; SEQ ID NOs: 26, 20, 10 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 20, 10 and 28, respectively; SEQ ID NOs: 21, 27, 11 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 21, 27, 11 and 29, respectively; SEQ ID NOs: 26, 10, 20 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 10, 20 and 28, respectively; SEQ ID NOs: 11, 27, 21 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 27, 21 and 29, respectively; SEQ ID NOs: 22, 6, 20 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 6, 20 and 28, respectively; SEQ ID NOs: 23, 7, 21 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 23, 7, 21 and 29, respectively; SEQ ID NOs: 22, 20, 6 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 20, 6 and 28, respectively; or SEQ ID NOs: 7, 21, 23 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 23 and 29, respectively. In some embodiments, the fusion polypeptide comprises or consists of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227.

In some embodiments, the fusion polypeptide comprises or consists of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223.

Further provided are compound fusion polypeptides comprising at least a first fusion polypeptide and a second fusion polypeptide, the first and second fusion polypeptides being independently selected from the fusion polypeptides as described above and herein. In some embodiments, the compound fusion polypeptide comprises or consists of the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the compound fusion polypeptide comprises or consists of the following first fusion polypeptide and second fusion polypeptide in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively; SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively. In some embodiments, the first fusion polypeptide and the second fusion polypeptide are joined or connected by a cleavable linker. In some embodiments, the first fusion polypeptide and the second fusion polypeptide are joined or connected by a cleavable linker selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. RAKR (SEQ ID NO: 60), REKR (SEQ ID NO: 61) and RRKR (SEQ ID NO: 62)), and combinations, derivatives or variants thereof. In some embodiments, the first fusion polypeptide and the second fusion polypeptide are joined or connected by a furin recognition/cleavage site selected from RAKR (SEQ ID NO: 60), REKR (SEQ ID NO: 61) and RRKR (SEQ ID NO: 62). In some embodiments, the first fusion polypeptide and the second fusion polypeptide are joined or connected by a 2A cleavable peptide comprising or consisting of the amino acid sequence of ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67, or having an amino acid sequence that is at least 90%, 91%, 92%, 93%, 94%, 95%, 96%, 97%, 98% or at least 99% identical to ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67. In some embodiments, the compound fusion polypeptide comprises or consists of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the compound fusion polypeptide comprises or consists of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223.

With respect to further embodiments of the fusion polypeptides and the compound fusion polypeptides, in some embodiments, the fusion polypeptide or the compound fusion polypeptide further comprises an N-terminal signal peptide or leader sequence. In some embodiments, the signal peptide or leader sequence is from a source protein selected from a serum protein, a cytokine, a chemokine, a chaperone protein, an invariant protein, and a protein that directs proteins to the lysosomal compartment. In some embodiments, the signal peptide or leader sequence is from a source protein selected from colony stimulating factor 2 (CSF2, GM-CSF), tissue type plasminogen activator (PLAT, t-PA), C-C motif chemokine ligand 7 (CCL7, MCP-3), C-X-C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2). In some embodiments, the signal peptide or leader sequence is selected from an amino acid sequence of any one of SEQ ID NOs: 115-126, or an amino acid sequence that is at least 95%, 96%, 97%, 98%, or 99% identical to any one of SEQ ID NOs: 115-126. In some embodiments, the fusion polypeptide and/or the compound fusion polypeptide are recombinantly produced or chemically synthesized. In some embodiments, the fusion polypeptide and/or the compound fusion polypeptide are capable of inducing, promoting or stimulating an immune response in a human. In some embodiments, the fusion polypeptide and/or the compound fusion polypeptide are capable of inducing, promoting or stimulating an immune response against HIV-1 in a human. In some embodiments, the fusion polypeptide and/or the compound fusion polypeptide are capable of inducing, promoting or stimulating proliferation and/or activation of one or more cell types selected from monocyte-derived dendritic cells (DCs), CD8+ T cells and CD4+T cells.

Further provided are polynucleotides encoding one or more fusion polypeptides or compound fusion polypeptide, as described above and herein. In some embodiments, the polynucleotide comprises cDNA, mRNA, self-amplifying RNA (SAM, saRNA), self-replicating RNA, or self-amplifying replicon RNA (RepRNA). In some embodiments, the polynucleotide comprises self-replicating or self-amplifying alphavirus replicons. In some embodiments, the polynucleotide comprises a nucleic acid sequence of any one of SEQ ID NOs: 130-167, 210-219 and 225-226, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the polynucleotide comprises a nucleic acid sequence of any one of SEQ ID NOs: 139, 140, 142, 143, 145, 146, 148, 149, 150, 152, 155, 158, 225 and 226, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226. Further provided are expression cassettes comprising one or more polynucleotides described herein operably linked to one or more regulatory sequences, e.g., a promoter. In some embodiments, the polynucleotide is operably linked to and under the control of a constitutive promoter. In some embodiments, the promoter is selected from a CMV promoter, a CAG promoter and an EFla promoter.

Further provided are lipoplexes, e.g., lipid nanoparticles (LNPs) comprising one or more polynucleotides described herein, e.g., encoding one or more polypeptides having an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227, e.g., one or more polynucleotides comprising a nucleic acid sequence of any one of SEQ ID NOs: 130-167, 210-219 and 225-226, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the lipoplexes, e.g., lipid nanoparticles (LNPs) comprise one or more polynucleotides described herein, e.g., encoding one or more polypeptides having an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223, e.g., one or more polynucleotides comprising a nucleic acid sequence of any one of SEQ ID NOs: 139, 140, 142, 143, 145, 146, 148, 149, 150, 152, 155, 158, 225 and 226, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226.

Further provided are vectors comprising one or more polynucleotides or one or more expression cassette, described herein. In some embodiments, the vector comprises or consists of one or more polynucleotides encoding one or more fusion polypeptides comprising the following polypeptide segments comprising in sequential order, from N-terminus to C terminus, optionally joined or connected by one or more linkers: SEQ ID NOs: 6, 4, 16 and 26, and SEQ ID NOs: 7, 5, 27 and 17; SEQ ID NOs: 12, 24, 8 and 10, and SEQ ID NOs: 9, 25, 13 and 11; SEQ ID NOs: 14, 22, 18, 24 and 20, and SEQ ID NOs: 15, 25, 19, 23 and 21; SEQ ID NOs: 26, 16, 4 and 6, and SEQ ID NOs: 7, 27, 5 and 17; SEQ ID NOs: 8, 24, 12 and 10, and SEQ ID NOs: 13, 25, 9 and 11; SEQ ID NOs: 22, 24, 14, 18 and 20, and SEQ ID NOs: 25, 23, 15, 21 and 19; SEQ ID NOs: 20, 6, 4, 18, 16 and 26, and SEQ ID NOs: 7, 19, 5, 21, 27 and 17; SEQ ID NOs: 8, 24, 14, 12, 22 and 10, and SEQ ID NOs: 9, 13, 25, 23, 15 and 11; SEQ ID NOs: 18, 26, 20, 4, 6 and 16, and SEQ ID NOs: 7, 21, 17, 5, 27 and 19; SEQ ID NOs: 22, 24, 12, 14, 8 and 10, and SEQ ID NOs: 15, 25, 9, 23, 13 and 11; SEQ ID NOs: 24, 6, 4, 20, 18 and 32, and SEQ ID NOs: 8, 30, 14, 12, 26 and 10; SEQ ID NOs: 24, 6, 4, 20, 18 and 32, and SEQ ID NOs: 7, 21, 5, 25, 33 and 19; SEQ ID NOs: 24, 6, 4, 20, 18 and 32, and SEQ ID NOs: 8, 30, 14, 12, 26 and 10; SEQ ID NOs: 8, 30, 14, 12, 26 and 10, and SEQ ID NOs: 9, 13, 31, 27, 15 and 11; SEQ ID NOs: 6, 20, 4, 24, 32 and 18, and SEQ ID NOs: 8, 12, 30, 26, 14 and 10; SEQ ID NOs: 7, 21, 5, 25, 33 and 19 and SEQ ID NOs: 9, 13, 31, 27, 15 and 11; SEQ ID NOs: 20, 32, 24, 4, 6 and 18, and SEQ ID NOs: 26, 30, 12, 14, 8 and 10; SEQ ID NOs: 7, 25, 19, 5, 33 and 21, and SEQ ID NOs: 15, 31, 9, 27, 13 and 11; SEQ ID NOs: 24, 6, 16 and 18, and SEQ ID NOs: 26, 20, 10 and 28; SEQ ID NOs: 24, 6, 16 and 18, and SEQ ID NOs: 26, 10, 20 and 28; SEQ ID NOs: 24, 6, 16 and 18, and SEQ ID NOs: 7, 25, 17 and 19; SEQ ID NOs: 7, 19, 17 and 25, and SEQ ID NOs: 21, 27, 11 and 29; SEQ ID NOs: 24, 16, 6 and 18, and SEQ ID NOs: 27, 11, 21 and 29; SEQ ID NOs: 7, 25, 17 and 19, and SEQ ID NOs: 11, 27, 21 and 29; SEQ ID NOs: 7, 25, 17 and 19, and SEQ ID NOs: 21, 27, 11 and 29; SEQ ID NOs: 22, 6, 20 and 28, and SEQ ID NOs: 23, 7, 21 and 29; SEQ ID NOs: 22, 20, 6 and 28, and SEQ ID NOs: 7, 21, 23 and 29; SEQ ID NOs: 26, 10, 20 and 28, and SEQ ID NOs: 21, 27, 11 and 29; SEQ ID NOs: 22, 6, 16, 20, 18, 28, 26 and 10; or SEQ ID NOs: 7, 21, 19, 17, 27, 25, 29 and 11. In some embodiments, the vector comprises one or more polynucleotides encoding one or more fusion polypeptides comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the vector comprises one or more polynucleotides encoding one or more fusion polypeptides comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223. In some embodiments, the vector comprises one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the vector comprises one or more polynucleotides encoding the following first fusion polypeptide and second fusion polypeptide in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively; SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively. In some embodiments, the vector comprises one or more polynucleotides encoding the following first fusion polypeptide and second fusion polypeptide, optionally joined or connected by one or more linkers: SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively. In some embodiments, the vector comprises one or more polynucleotides encoding the following first fusion polypeptide and second fusion polypeptide, optionally joined or connected by one or more linkers: SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively. In some embodiments, the vector comprises one or more polynucleotides encoding the following first fusion polypeptide and second fusion polypeptide, optionally joined or connected by one or more linkers: SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively. In some embodiments, the vector comprises one or more polynucleotides encoding the following first fusion polypeptide and second fusion polypeptide, optionally joined or connected by one or more linkers: SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively. In some embodiments, the vector comprises one or more polynucleotides encoding the following first fusion polypeptide and second fusion polypeptide, optionally joined or connected by one or more linkers: SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively. In some embodiments, the vector comprises one or more polynucleotides encoding the following first fusion polypeptide and second fusion polypeptide, optionally joined or connected by one or more linkers: SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively. In some embodiments, the vector comprises one or more polynucleotides encoding the following first fusion polypeptide and second fusion polypeptide, optionally joined or connected by one or more linkers: SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively. In some embodiments, the vector comprises one or more polynucleotides encoding the following first fusion polypeptide and second fusion polypeptide, optionally joined or connected by one or more linkers: SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the vector comprises one or more polynucleotides encoding the following first fusion polypeptide and second fusion polypeptide, optionally joined or connected by one or more linkers: SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the vector comprises one or more polynucleotides encoding the following first fusion polypeptide and second fusion polypeptide, optionally joined or connected by one or more linkers: SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively. In some embodiments, the vector comprises one or more polynucleotides encoding the following first fusion polypeptide and second fusion polypeptide, optionally joined or connected by one or more linkers: SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively. In some embodiments, the vector comprises one or more polynucleotides encoding the following first fusion polypeptide and second fusion polypeptide, optionally joined or connected by one or more linkers: SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively. In some embodiments, the vector comprises one or more polynucleotides encoding the following first fusion polypeptide and second fusion polypeptide, optionally joined or connected by one or more linkers: SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In some embodiments, the vector comprises one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively. In some embodiments, the vector comprises one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively. In some embodiments, the vector comprises one or more polynucleotides encoding the following first fusion polypeptide and second fusion polypeptide, optionally joined or connected by one or more linkers: SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively. In some embodiments, the vector comprises one or more polynucleotides encoding the following first fusion polypeptide and second fusion polypeptide, optionally joined or connected by one or more linkers: SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively. In some embodiments, the vector comprises one or more polynucleotides encoding the following first fusion polypeptide and second fusion polypeptide, optionally joined or connected by one or more linkers: SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the vector comprises one or more polynucleotides encoding the following first fusion polypeptide and second fusion polypeptide, optionally joined or connected by one or more linkers: SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively. In some embodiments, the vector comprises one or more polynucleotides encoding the following first fusion polypeptide and second fusion polypeptide, optionally joined or connected by one or more linkers: SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the vector comprises one or more polynucleotides encoding the following first fusion polypeptide and second fusion polypeptide, optionally joined or connected by one or more linkers: SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively. In some embodiments, the vector comprises one or more polynucleotides encoding the following first fusion polypeptide and second fusion polypeptide, optionally joined or connected by one or more linkers: SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively. In some embodiments, the vector comprises one or more polynucleotides encoding the following first fusion polypeptide and second fusion polypeptide, optionally joined or connected by one or more linkers: SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the vector comprises one or more polynucleotides encoding the following first fusion polypeptide and second fusion polypeptide, optionally joined or connected by one or more linkers: SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the vector comprises a polynucleotide encoding a compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the vector comprises a polynucleotide encoding a compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the first and second fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In some embodiments, the first and second fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof. In some embodiments, the vector comprises a polynucleotide comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the vector comprises a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226. In some embodiments, the following first polynucleotide and second polynucleotide: SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively; SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively; SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively; SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively; SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively; SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively; SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively; SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively; SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively; SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively; SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively; SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively; SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively; SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively; SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively; SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively; SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively; SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively; SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively; SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively; SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively; SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively; SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively; SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively; SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively; SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively. In some embodiments, the vector comprises the following first polynucleotide and second polynucleotide: (a) a first polynucleotide comprising SEQ ID NOs: 130 or SEQ ID NO: 131, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 130 or SEQ ID NO: 131, respectively, and (b) a second polynucleotide comprising SEQ ID NO: 134 or SEQ ID NO: 135, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 134 or SEQ ID NO: 135. In some embodiments, the vector comprises the following first polynucleotide and second polynucleotide: (a) a first polynucleotide comprising SEQ ID NO: 132 or SEQ ID NO: 133, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 132 or SEQ ID NO: 133, respectively, and (b) a second polynucleotide comprising SEQ ID NO: 136 or SEQ ID NO: 137, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 136 or SEQ ID NO: 137. In some embodiments, the vector comprises the following first polynucleotide and second polynucleotide: (a) a first polynucleotide comprising SEQ ID NO: 139, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 139, respectively, and (b) a second polynucleotide comprising SEQ ID NO: 145, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 145. In some embodiments, the vector comprises the following first polynucleotide and second polynucleotide: (a) a first polynucleotide comprising SEQ ID NO: 142, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 142, respectively, and (b) a second polynucleotide comprising SEQ ID NO: 148, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 148. In some embodiments, the vector comprises the following first polynucleotide and second polynucleotide: (a) a first polynucleotide comprising SEQ ID NO: 140, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 140, respectively, and (b) a second polynucleotide comprising SEQ ID NO: 146, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 146. In some embodiments, the vector comprises the following first polynucleotide and second polynucleotide: (a) a first polynucleotide comprising SEQ ID NO: 143, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 143, respectively, and (b) a second polynucleotide comprising SEQ ID NO: 149, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 149. In some embodiments, the vector comprises the following first polynucleotide and second polynucleotide: (a) a first polynucleotide comprising SEQ ID NO: 150, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 150, respectively, and (b) a second polynucleotide comprising SEQ ID NO: 152, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 152. In some embodiments, the vector comprises the following first polynucleotide and second polynucleotide: (a) a first polynucleotide comprising SEQ ID NO: 151, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 151, respectively, and (b) a second polynucleotide comprising SEQ ID NO: 153, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 153. In some embodiments, the vector comprises the following first polynucleotide and second polynucleotide: (a) a first polynucleotide comprising SEQ ID NO: 154, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 154, respectively, and (b) a second polynucleotide comprising SEQ ID NO: 157, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 157. In some embodiments, the vector comprises the following first polynucleotide and second polynucleotide: (a) a first polynucleotide comprising SEQ ID NO: 155, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 155, respectively, and (b) a second polynucleotide comprising SEQ ID NO: 158, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 158. In some embodiments, the vector comprises the following first polynucleotide and second polynucleotide: (a) a first polynucleotide comprising SEQ ID NO: 156, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 156, respectively, and (b) a second polynucleotide comprising SEQ ID NO: 159, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 159. In some embodiments, the vector comprises the following first polynucleotide and second polynucleotide: (a) a first polynucleotide comprising SEQ ID NO: 160, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 160, respectively, and (b) a second polynucleotide comprising SEQ ID NO: 161, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 161. In some embodiments, the vector comprises the following first polynucleotide and second polynucleotide: (a) a first polynucleotide comprising SEQ ID NO: 162, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 162, respectively, and (b) a second polynucleotide comprising SEQ ID NO: 163, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 163. In some embodiments, the vector comprises the following first polynucleotide and second polynucleotide: (a) a first polynucleotide comprising SEQ ID NO: 164, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 164, respectively, and (b) a second polynucleotide comprising SEQ ID NO: 165, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 165. In some embodiments, the vector comprises the following first polynucleotide and second polynucleotide: (a) a first polynucleotide comprising SEQ ID NO: 166, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 166, respectively, and (b) a second polynucleotide comprising SEQ ID NO: 167, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 167. In some embodiments, the vector comprises the following first polynucleotide and second polynucleotide: (a) a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and (b) a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211. In some embodiments, the vector comprises the following first polynucleotide and second polynucleotide: (a) a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and (b) a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213. In some embodiments, the vector comprises the following first polynucleotide and second polynucleotide: (a) a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and (b) a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215. In some embodiments, the vector comprises the following first polynucleotide and second polynucleotide: (a) a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and (b) a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217. In some embodiments, the vector comprises the following first polynucleotide and second polynucleotide: (a) a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219. In some embodiments, the vector comprises the following first polynucleotide and second polynucleotide: (a) a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the vector comprises the following first polynucleotide and second polynucleotide: (a) a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and (b) a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the first polynucleotide encoding the first fusion polypeptide and the second polynucleotide encoding the second fusion polypeptide are in a single open reading frame. In some embodiments, the first polynucleotide encoding the first fusion polypeptide is in a first open reading frame and the second polynucleotide encoding the second fusion polypeptide is in a second open reading frame. In some embodiments, the first polynucleotide encoding the first fusion polypeptide and the second polynucleotide encoding the second fusion polypeptide are in a single expression cassette. In some embodiments, the first polynucleotide encoding the first fusion polypeptide is in a first expression cassette and the second polynucleotide encoding the second fusion polypeptide is in a second expression cassette. In some embodiments, the vector is a plasmid vector, a bacterial vector or a viral vector. In some embodiments, the vector is a viral vector. In some embodiments, the viral vector is a DNA virus or an RNA virus. In some embodiments, the viral vector is replication defective, replication deficient, replication attenuated or replication competent. In some embodiments, the viral vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral vector is a human adenovirus or a simian adenovirus (e.g., a chimpanzee adenovirus, a gorilla adenovirus or a rhesus adenovirus). In some embodiments, the viral vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (e.g. ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), ChAd147 (AdC147)), gorilla adenovirus (e.g. GC44, GC45, GC46) and rhesus adenovirus (e.g., RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66).

Further provided are host cells comprising one or more polynucleotides, one or more expression cassettes, or one or more vectors, as described herein. In some embodiments, the one or more polynucleotides are not integrated into the host cell genome, e.g., are episomal. In some embodiments, the one or more polynucleotides are integrated into the host cell genome. In some embodiments, the host cell is a mammalian cell, e.g., a human cell. In various embodiments, the host cell can be in vitro or in vivo.

Further provided are immunogenic compositions. In various embodiments, the immunogenic compositions comprise one or more of the fusion polypeptides or compound fusion polypeptides, one or more polynucleotides, or one or more vectors, as described herein, and a pharmaceutically acceptable carrier. In some embodiments, the immunogenic composition comprises two or more of the fusion polypeptides or compound fusion polypeptides, two or more polynucleotides, two or more vectors, as described herein. In some embodiments, the one or more polynucleotides are DNA, cDNA, mRNA, or self-replicating RNA. In some embodiments, the immunogenic composition comprises a first fusion polypeptide and a second fusion polypeptide, or one or more vectors comprising one or more polynucleotides encoding the first and second fusion polypeptides, the first and second polypeptides comprising the following polypeptide segments, in sequential order, from N-terminus to C terminus, optionally joined or connected by one or more linkers: SEQ ID NOs: 6, 4, 16 and 26, and SEQ ID NOs: 7, 5, 27 and 17; SEQ ID NOs: 12, 24, 8 and 10, and SEQ ID NOs: 9, 25, 13 and 11; SEQ ID NOs: 14, 22, 18, 24 and 20, and SEQ ID NOs: 15, 25, 19, 23 and 21; SEQ ID NOs: 26, 16, 4 and 6, and SEQ ID NOs: 7, 27, 5 and 17; SEQ ID NOs: 8, 24, 12 and 10, and SEQ ID NOs: 13, 25, 9 and 11; SEQ ID NOs: 22, 24, 14, 18 and 20, and SEQ ID NOs: 25, 23, 15, 21 and 19; SEQ ID NOs: 20, 6, 4, 18, 16 and 26, and SEQ ID NOs: 7, 19, 5, 21, 27 and 17; SEQ ID NOs: 8, 24, 14, 12, 22 and 10, and SEQ ID NOs: 9, 13, 25, 23, 15 and 11; SEQ ID NOs: 18, 26, 20, 4, 6 and 16, and SEQ ID NOs: 7, 21, 17, 5, 27 and 19; SEQ ID NOs: 22, 24, 12, 14, 8 and 10, and SEQ ID NOs: 15, 25, 9, 23, 13 and 11; SEQ ID NOs: 24, 6, 4, 20, 18 and 32, and SEQ ID NOs: 8, 30, 14, 12, 26 and 10; SEQ ID NOs: 24, 6, 4, 20, 18 and 32, and SEQ ID NOs: 7, 21, 5, 25, 33 and 19; SEQ ID NOs: 24, 6, 4, 20, 18 and 32, and SEQ ID NOs: 8, 30, 14, 12, 26 and 10; SEQ ID NOs: 8, 30, 14, 12, 26 and 10, and SEQ ID NOs: 9, 13, 31, 27, 15 and 11; SEQ ID NOs: 6, 20, 4, 24, 32 and 18, and SEQ ID NOs: 8, 12, 30, 26, 14 and 10; SEQ ID NOs: 7, 21, 5, 25, 33 and 19 and SEQ ID NOs: 9, 13, 31, 27, 15 and 11; SEQ ID NOs: 20, 32, 24, 4, 6 and 18, and SEQ ID NOs: 26, 30, 12, 14, 8 and 10; SEQ ID NOs: 7, 25, 19, 5, 33 and 21, and SEQ ID NOs: 15, 31, 9, 27, 13 and 11; SEQ ID NOs: 24, 6, 16 and 18, and SEQ ID NOs: 26, 20, 10 and 28; SEQ ID NOs: 24, 6, 16 and 18, and SEQ ID NOs: 26, 10, 20 and 28; SEQ ID NOs: 24, 6, 16 and 18, and SEQ ID NOs: 7, 25, 17 and 19; SEQ ID NOs: 7, 19, 17 and 25, and SEQ ID NOs: 21, 27, 11 and 29; SEQ ID NOs: 24, 16, 6 and 18, and SEQ ID NOs: 27, 11, 21 and 29; SEQ ID NOs: 7, 25, 17 and 19, and SEQ ID NOs: 11, 27, 21 and 29; SEQ ID NOs: 7, 25, 17 and 19, and SEQ ID NOs: 21, 27, 11 and 29; SEQ ID NOs: 22, 6, 20 and 28, and SEQ ID NOs: 23, 7, 21 and 29; SEQ ID NOs: 22, 20, 6 and 28, and SEQ ID NOs: 7, 21, 23 and 29; SEQ ID NOs: 26, 10, 20 and 28, and SEQ ID NOs: 21, 27, 11 and 29; SEQ ID NOs: 22, 6, 16, 20, 18, 28, 26 and 10; or SEQ ID NOs: 7, 21, 19, 17, 27, 25, 29 and 11. In some embodiments, the immunogenic composition comprises one or more fusion polypeptides, or one or more vectors comprising one or more polynucleotides encoding one or more fusion polypeptides, comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the immunogenic composition comprises one or more fusion polypeptides, or one or more vectors comprising one or more polynucleotides encoding one or more fusion polypeptides, comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223. In some embodiments, the immunogenic composition comprises the following first fusion polypeptide and second fusion polypeptide, one or more polynucleotides, or one or more vectors or one or more lipoplexes (e.g., LNPs) comprising one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the immunogenic composition comprises the following first fusion polypeptide and second fusion polypeptide, one or more polynucleotides, or one or more vectors or one or more lipoplexes (e.g., LNPs) comprising one or more polynucleotides encoding the following first fusion polypeptide and second fusion polypeptide, in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively; SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively. In some embodiments, the immunogenic composition comprises first and second polynucleotides, first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively, and (b) a second polynucleotide or second viral vector or second lipoplex (e.g., LNP) comprising one or more polynucleotides encoding a first fusion polypeptide and a second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively, and (b) a second polynucleotide or second viral vector or second lipoplex (e.g., LNP) comprising one or more polynucleotides encoding a first fusion polypeptide and a second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively, and (b) a second polynucleotide or second viral vector or second lipoplex (e.g., LNP) comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the immunogenic composition comprises first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively, and (b) a second polynucleotide or second viral vector or second lipoplex (e.g., LNP) comprising one or more polynucleotides encoding a first fusion polypeptide and a second fusion polypeptide comprising SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively. In some embodiments, the immunogenic composition comprises first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively, and (b) a second polynucleotide or second viral vector or second lipoplex (e.g., LNP) comprising one or more polynucleotides encoding a first fusion polypeptide and a second fusion polypeptide comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively, and (b) a second polynucleotide or second viral vector or second lipoplex (e.g., LNP) comprising one or more polynucleotides encoding a first fusion polypeptide and a second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second polynucleotide or second viral vector or second lipoplex (e.g., LNP) comprising one or more polynucleotides encoding a first fusion polypeptide and a second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In some embodiments, the immunogenic composition comprises a viral vector or a lipoplex (e.g., LNP) comprising one or more polynucleotides encoding the following first fusion polypeptide and second fusion polypeptide, optionally joined or connected by one or more linkers, SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively. In some embodiments, the immunogenic composition comprises a viral vector or a lipoplex (e.g., LNP) comprising one or more polynucleotides encoding the following first fusion polypeptide and second fusion polypeptide, optionally joined or connected by one or more linkers, SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively. In some embodiments, the composition comprises first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively, and (b) a second polynucleotide or second viral vector or second lipoplex (e.g., LNP) comprising one or more polynucleotides encoding a first fusion polypeptide and a second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and (b) a second polynucleotide or second viral vector or second lipoplex (e.g., LNP) comprising one or more polynucleotides encoding a first fusion polypeptide and a second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and (b) a second polynucleotide or second viral vector or second lipoplex (e.g., LNP) comprising one or more polynucleotides encoding a first fusion polypeptide and a second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the immunogenic composition comprises first and second polynucleotides or first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second polynucleotide or second viral vector or second lipoplex (e.g., LNP) comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively. In some embodiments, the immunogenic composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the immunogenic composition comprises a first viral vector or a lipoplex (e.g., LNP) comprising a first polynucleotide encoding a first polypeptide comprising SEQ ID NO: 200, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, and a second viral vector or a lipoplex (e.g., LNP) comprising a second polynucleotide encoding a second polypeptide comprising SEQ ID NO: 201, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201. In some embodiments, the immunogenic composition comprises a first viral vector or a lipoplex (e.g., LNP) comprising a first polynucleotide encoding a first polypeptide comprising SEQ ID NO: 202, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202, and a second viral vector or a lipoplex (e.g., LNP) comprising a second polynucleotide encoding a second polypeptide comprising SEQ ID NO: 203, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203. In some embodiments, the immunogenic composition comprises a first viral vector or a lipoplex (e.g., LNP) comprising a first polynucleotide encoding a first polypeptide comprising SEQ ID NO: 203, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203, and a second viral vector or a lipoplex (e.g., LNP) comprising a second polynucleotide encoding a second polypeptide comprising SEQ ID NO: 204, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204. In some embodiments, the immunogenic composition comprises a first viral vector or a lipoplex (e.g., LNP) comprising a first polynucleotide encoding a first polypeptide comprising SEQ ID NO: 105, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, and a second viral vector or a lipoplex (e.g., LNP) comprising a second polynucleotide encoding a second polypeptide comprising SEQ ID NO: 107, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107. In some embodiments, the immunogenic composition comprises a first viral vector or a lipoplex (e.g., LNP) comprising a first polynucleotide encoding a first polypeptide comprising SEQ ID NO: 206, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, and a second viral vector or a lipoplex (e.g., LNP) comprising a second polynucleotide encoding a second polypeptide comprising SEQ ID NO: 207, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207. In some embodiments, the immunogenic composition comprises a first viral vector or a lipoplex (e.g., LNP) comprising a first polynucleotide encoding a first polypeptide comprising SEQ ID NO: 208, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, and a second viral vector or a lipoplex (e.g., LNP) comprising a second polynucleotide encoding a second polypeptide comprising SEQ ID NO: 209 or SEQ ID NO: 227, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the immunogenic composition comprises a first viral vector or a lipoplex (e.g., LNP) comprising a first polynucleotide encoding a first polypeptide comprising SEQ ID NO: 222, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and a second viral vector or a lipoplex (e.g., LNP) comprising a second polynucleotide encoding a second polypeptide comprising SEQ ID NO: 209 or SEQ ID NO: 227, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the immunogenic composition comprises a first viral vector or a lipoplex (e.g., LNP) comprising a first polynucleotide encoding a first polypeptide comprising SEQ ID NO: 222, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and a second viral vector or a lipoplex (e.g., LNP) comprising a second polynucleotide encoding a second polypeptide comprising SEQ ID NO: 223, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In some embodiments, the immunogenic composition comprises a polynucleotide comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the immunogenic composition comprises a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226. In some embodiments, the immunogenic composition comprises the following first polynucleotide and second polynucleotide, or one or more vectors comprising the following first polynucleotide and second polynucleotide: SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively; SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively; SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively; SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively; SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively; SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively; SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively; SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively; SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively; SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively; SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively; SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively; SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively; SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively; SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively; SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively; SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively; SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively; SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively; SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively; SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively; SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively; SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively; SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively; SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively; SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively. In some embodiments, the immunogenic composition comprises first and second polynucleotides or first and second viral vectors or first and second lipoplexes (e.g., LNPs) comprising the following first polynucleotide and second polynucleotide: (a) a first vector or first lipoplex (e.g., LNP) comprising first and second polynucleotides comprising SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively, and (b) a second vector or second lipoplex (e.g., LNP) comprising first and second polynucleotides comprising SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively. In some embodiments, the immunogenic composition comprises first and second polynucleotides or first and second viral vectors or first and second lipoplexes (e.g., LNPs) comprising the following first polynucleotide and second polynucleotide: (a) a first vector or first lipoplex (e.g., LNP) comprising first and second polynucleotides comprising SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively, and (b) a second vector or second lipoplex (e.g., LNP) comprising first and second polynucleotides comprising SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In some embodiments, the immunogenic composition comprises first and second polynucleotides or first and second viral vectors or first and second lipoplexes (e.g., LNPs) comprising the following first polynucleotide and second polynucleotide: (a) a first vector or first lipoplex (e.g., LNP) comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively, and (b) a second vector or second lipoplex (e.g., LNP) comprising first and second polynucleotides comprising SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively. In some embodiments, the first and second viral vectors of such an immunogenic composition are Lymphocytic choriomeningitis mammarenavirus (LCMV) viral vectors. In some embodiments, the immunogenic composition comprises first and second polynucleotides or first and second viral vectors or first and second lipoplexes (e.g., LNPs) comprising the following first polynucleotide and second polynucleotide: (a) a first vector or first lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and (b) a second vector or second lipoplex (e.g., LNP) comprising first and second polynucleotides comprising SEQ ID NOs: 154 and 157 or SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In some embodiments, the first and second viral vectors of such an immunogenic composition are Cali mammarenavirus (a.k.a. Pichinde mammarenavirus or Pichinde arenavirus) viral vectors. In some embodiments, the immunogenic composition comprises first and second polynucleotides or first and second viral vectors or first and second lipoplexes (e.g., LNPs) comprising the following first polynucleotide and second polynucleotide: (a) a first vector or first lipoplex (e.g., LNP) comprising first and second polynucleotides comprising SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively, and (b) a second vector or second lipoplex (e.g., LNP) comprising first and second polynucleotides comprising SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 160, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 160, respectively, and (b) the second viral vector or second lipoplex (e.g., LNP) comprising a polynucleotide of SEQ ID NO: 161, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 161, respectively. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 162, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 162, respectively, and (b) the second viral vector or second lipoplex (e.g., LNP) comprising a polynucleotide of SEQ ID NO: 163, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 163, respectively. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 164, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 164, respectively, and (b) the second viral vector or second lipoplex (e.g., LNP) comprising a polynucleotide of SEQ ID NO: 165, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 165, respectively. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 166, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 166, respectively, and (b) the second viral vector or second lipoplex (e.g., LNP) comprising a polynucleotide of SEQ ID NO: 167, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 167, respectively. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and (b) the second viral vector or second lipoplex (e.g., LNP) comprising a polynucleotide of SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and (b) the second viral vector or second lipoplex (e.g., LNP) comprising a polynucleotide of SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and (b) the second viral vector or second lipoplex (e.g., LNP) comprising a polynucleotide of SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and (b) the second viral vector or second lipoplex (e.g., LNP) comprising a polynucleotide of SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) the second viral vector or second lipoplex (e.g., LNP) comprising a polynucleotide of SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) the second viral vector or second lipoplex (e.g., LNP) comprising a polynucleotide of SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the immunogenic composition comprises first and second polynucleotides or first and second viral vectors or first and second lipoplexes (e.g., LNPs) comprising one or more polynucleotides: (a) the first viral vector or first lipoplex (e.g., LNP) comprising a polynucleotide of SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and (b) the second viral vector or second lipoplex (e.g., LNP) comprising a polynucleotide of SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the immunogenic composition comprises a compound fusion polypeptide, a vector or a lipoplex (e.g., LNP) comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the immunogenic composition comprises a compound fusion polypeptide, a vector or a lipoplex (e.g., LNP) comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In some embodiments, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof. In various embodiments of the immunogenic compositions, the first and second viral vectors can be Lymphocytic choriomeningitis mammarenavirus (LCMV) viral vectors, Cali mammarenavirus (a.k.a. Pichinde mammarenavirus or Pichinde arenavirus) viral vectors or adenoviral vectors, e.g., chimpanzee adenoviral vectors (ChAds). In some embodiments, the immunogenic composition comprises a single vector comprising one or more polynucleotides encoding first and second fusion polypeptides. In some embodiments, the immunogenic composition further comprises one or more of an adjuvant, a detergent, a micelle-forming agent, and an oil. In some embodiments, the immunogenic composition is formulated for administration via a route selected from intravenous, intramuscular, intradermal, subcutaneous, intranodal and mucosal (e.g. buccal, intranasal, intrarectal, intravaginal). In some embodiments, the immunogenic composition is formulated as a liquid, a suspension or an emulsion. In some embodiments, the immunogenic composition is lyophilized.

Further provided are kits comprising one or more unitary doses of one or more of the fusion polypeptides, one or more compound fusion polypeptides, one or more polynucleotides, one or more vectors, or one or more immunogenic compositions, as described herein. In some embodiments, the kit comprises two or more of the fusion polypeptides, two or more compound fusion polypeptides, two or more polynucleotides, two or more vectors, or two or more immunogenic compositions, as described herein. In some embodiments, the one or more unitary doses are in a single container. In some embodiments, one or more unitary doses are in two or more separate containers. In some embodiments, the kit comprises one or more containers selected from vials, ampules and pre-loaded syringes. In some embodiments, the kit comprises one or more containers comprising the one or more fusion polypeptides, one or more polynucleotides or one or more vectors in an aqueous solution. In some embodiments, the one or more unitary doses are the same. In some embodiments, the one or more unitary doses are the different. In some embodiments, the kit comprises one or more unitary doses of one or more viral vectors and the unitary doses are in the range of about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp. In some embodiments, the kit comprises a first fusion polypeptide and a second fusion polypeptide, or one or more vectors comprising one or more polynucleotides encoding the first and second fusion polypeptides, the first and second polypeptides comprising the following polypeptide segments, in sequential order, from N-terminus to C terminus, optionally joined or connected by one or more linkers: SEQ ID NOs: 6, 4, 16 and 26, and SEQ ID NOs: 7, 5, 27 and 17; SEQ ID NOs: 12, 24, 8 and 10, and SEQ ID NOs: 9, 25, 13 and 11; SEQ ID NOs: 14, 22, 18, 24 and 20, and SEQ ID NOs: 15, 25, 19, 23 and 21; SEQ ID NOs: 26, 16, 4 and 6, and SEQ ID NOs: 7, 27, 5 and 17; SEQ ID NOs: 8, 24, 12 and 10, and SEQ ID NOs: 13, 25, 9 and 11; SEQ ID NOs: 22, 24, 14, 18 and 20, and SEQ ID NOs: 25, 23, 15, 21 and 19; SEQ ID NOs: 20, 6, 4, 18, 16 and 26, and SEQ ID NOs: 7, 19, 5, 21, 27 and 17; SEQ ID NOs: 8, 24, 14, 12, 22 and 10, and SEQ ID NOs: 9, 13, 25, 23, 15 and 11; SEQ ID NOs: 18, 26, 20, 4, 6 and 16, and SEQ ID NOs: 7, 21, 17, 5, 27 and 19; SEQ ID NOs: 22, 24, 12, 14, 8 and 10, and SEQ ID NOs: 15, 25, 9, 23, 13 and 11; SEQ ID NOs: 24, 6, 4, 20, 18 and 32, and SEQ ID NOs: 8, 30, 14, 12, 26 and 10; SEQ ID NOs: 24, 6, 4, 20, 18 and 32, and SEQ ID NOs: 7, 21, 5, 25, 33 and 19; SEQ ID NOs: 24, 6, 4, 20, 18 and 32, and SEQ ID NOs: 8, 30, 14, 12, 26 and 10; SEQ ID NOs: 8, 30, 14, 12, 26 and 10, and SEQ ID NOs: 9, 13, 31, 27, 15 and 11; SEQ ID NOs: 6, 20, 4, 24, 32 and 18, and SEQ ID NOs: 8, 12, 30, 26, 14 and 10; SEQ ID NOs: 7, 21, 5, 25, 33 and 19 and SEQ ID NOs: 9, 13, 31, 27, 15 and 11; SEQ ID NOs: 20, 32, 24, 4, 6 and 18, and SEQ ID NOs: 26, 30, 12, 14, 8 and 10; SEQ ID NOs: 7, 25, 19, 5, 33 and 21, and SEQ ID NOs: 15, 31, 9, 27, 13 and 11; SEQ ID NOs: 24, 6, 16 and 18, and SEQ ID NOs: 26, 20, 10 and 28; SEQ ID NOs: 24, 6, 16 and 18, and SEQ ID NOs: 26, 10, 20 and 28; SEQ ID NOs: 24, 6, 16 and 18, and SEQ ID NOs: 7, 25, 17 and 19; SEQ ID NOs: 7, 19, 17 and 25, and SEQ ID NOs: 21, 27, 11 and 29; SEQ ID NOs: 24, 16, 6 and 18, and SEQ ID NOs: 27, 11, 21 and 29; SEQ ID NOs: 7, 25, 17 and 19, and SEQ ID NOs: 11, 27, 21 and 29; SEQ ID NOs: 7, 25, 17 and 19, and SEQ ID NOs: 21, 27, 11 and 29; SEQ ID NOs: 22, 6, 20 and 28, and SEQ ID NOs: 23, 7, 21 and 29; SEQ ID NOs: 22, 20, 6 and 28, and SEQ ID NOs: 7, 21, 23 and 29; SEQ ID NOs: 26, 10, 20 and 28, and SEQ ID NOs: 21, 27, 11 and 29; SEQ ID NOs: 22, 6, 16, 20, 18, 28, 26 and 10; or SEQ ID NOs: 7, 21, 19, 17, 27, 25, 29 and 11. In some embodiments, the kit comprises one or more fusion polypeptides, one or more vectors, or one or more lipoplexes (LNPs), comprising one or more polynucleotides encoding one or more fusion polypeptides, comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the kit comprises one or more fusion polypeptides, one or more vectors, or one or more lipoplexes (LNPs), comprising one or more polynucleotides encoding one or more fusion polypeptides, comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223. In some embodiments, the kit comprises the following first fusion polypeptide and second fusion polypeptide, or one or more vectors comprising one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the kit comprises the following first fusion polypeptide and second fusion polypeptide, one or more vectors, or one or more lipoplexes (e.g., LNPs), comprising one or more polynucleotides encoding the following first fusion polypeptide and second fusion polypeptide, in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively; SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; (b) a second viral vector or lipoplex (e.g., LNP) comprising one or more polynucleotides encoding a first fusion polypeptide and a second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; (c) a third viral vector or lipoplex (e.g., LNP) comprising one or more polynucleotides encoding a first fusion polypeptide and a second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; and (d) a fourth viral vector or lipoplex (e.g., LNP) comprising one or more polynucleotides encoding a first fusion polypeptide and a second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively, and (b) a second viral vector or lipoplex (e.g., LNP) comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively, and (b) a second viral vector or lipoplex (e.g., LNP) comprising one or more polynucleotides encoding a first fusion polypeptide and a second fusion polypeptide comprising SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively, and (b) a second viral vector or lipoplex (e.g., LNP) comprising one or more polynucleotides encoding a first fusion polypeptide and a second fusion polypeptide comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 200, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, and (b) a second viral vector or lipoplex (e.g., LNP) comprising a polynucleotide encoding a fusion polypeptide comprising SEQ ID NO: 201, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 202, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202, and (b) a second viral vector or lipoplex (e.g., LNP) comprising a polynucleotide encoding a fusion polypeptide comprising SEQ ID NO: 203, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 204, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204, and (b) a second viral vector or lipoplex (e.g., LNP) comprising a polynucleotide encoding a fusion polypeptide comprising SEQ ID NO: 205, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 105 or 206, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105 or 206, respectively, and (b) a second viral vector or lipoplex (e.g., LNP) comprising a polynucleotide encoding a fusion polypeptide comprising SEQ ID NO: 107 or 207, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107 or 207, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 208, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, and (b) a second viral vector or lipoplex (e.g., LNP) comprising a polynucleotide encoding a fusion polypeptide comprising SEQ ID NO: 209 or SEQ ID NO: 227, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 222, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second viral vector or lipoplex (e.g., LNP) comprising a polynucleotide encoding a fusion polypeptide comprising SEQ ID NO: 209 or SEQ ID NO: 227, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising a polynucleotide encoding a fusion polypeptide comprising SEQ ID NO: 222, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second viral vector or lipoplex (e.g., LNP) comprising a polynucleotide encoding a fusion polypeptide comprising SEQ ID NO: 223, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; (b) a second viral vector or lipoplex (e.g., LNP) comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; (c) a third viral vector or lipoplex (e.g., LNP) comprising one or more polynucleotides encoding a first fusion polypeptide and a second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; and (d) a fourth viral vector or lipoplex (e.g., LNP) comprising one or more polynucleotides encoding a first fusion polypeptide and a second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; (b) a second viral vector or lipoplex (e.g., LNP) comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; (c) a third viral vector or lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (d) a fourth viral vector or lipoplex (e.g., LNP) comprising one or more polynucleotides encoding a first fusion polypeptide and a second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; (b) a second viral vector or lipoplex (e.g., LNP) comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; (c) a third viral vector or lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (d) a fourth viral vector or lipoplex (e.g., LNP) comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively, and (b) a second viral vector or lipoplex (e.g., LNP) comprising one or more polynucleotides encoding a first fusion polypeptide and a second fusion polypeptide comprising SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding the following first fusion polypeptide and second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and (b) a second viral vector or lipoplex (e.g., LNP) comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding the following first fusion polypeptide and second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and (b) a second viral vector or lipoplex (e.g., LNP) comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding the following first fusion polypeptide and second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively, and (b) a second viral vector or lipoplex (e.g., LNP) comprising one or more polynucleotides encoding the following first fusion polypeptide and second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the kit comprises first and second viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second viral vector or lipoplex (e.g., LNP) comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; (b) a second viral vector or lipoplex (e.g., LNP) comprising one or more polynucleotides encoding a first fusion polypeptide and a second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; (c) a third viral vector or lipoplex (e.g., LNP) comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (d) a fourth viral vector or lipoplex (e.g., LNP) comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the kit comprises first, second, third and fourth viral vectors or lipoplexes (e.g., LNPs) comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding a first fusion polypeptide and a second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; (b) a second viral vector or lipoplex (e.g., LNP) comprising one or more polynucleotides encoding a first fusion polypeptide and a second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; (c) a third viral vector or lipoplex (e.g., LNP) comprising one or more polynucleotides encoding a first fusion polypeptide and a second fusion polypeptide comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; and (d) a fourth viral vector or lipoplex (e.g., LNP) comprising one or more polynucleotides encoding a first fusion polypeptide and a second fusion polypeptide comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In various embodiments of the kits, the viral vectors are Lymphocytic choriomeningitis mammarenavirus (LCMV) viral vectors, Cali mammarenavirus (a.k.a. Pichinde mammarenavirus or Pichinde arenavirus) viral vectors or adenoviral vectors, e.g., chimpanzee adenoviral vectors (ChAds). In some embodiments, the kit comprises a polynucleotide comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the kit comprises a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226. In some embodiments, the kit comprises the following first polynucleotide and second polynucleotide, one or more vectors, or one or more lipoplexes (e.g., LNPs) comprising the following first polynucleotide and second polynucleotide: SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively; SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively; SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively; SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively; SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively; SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively; SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively; SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively; SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively; SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively; SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively; SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively; SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively; SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively; SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively; SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively; SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively; SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively; SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively; SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively; SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively; SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively; SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively; SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively; SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively; SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively. In some embodiments, the kit comprises first and second viral vectors or lipoplexes (e.g., LNPs) comprising the following first polynucleotide and second polynucleotide: (a) a first vector or lipoplex (e.g., LNP) comprising first and second polynucleotides comprising SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively, and (b) a second vector or lipoplex (e.g., LNP) comprising first and second polynucleotides comprising SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively. In some embodiments, the kit comprises first and second viral vectors or lipoplexes (e.g., LNPs) comprising the following first polynucleotide and second polynucleotide: (a) a first vector or lipoplex (e.g., LNP) comprising first and second polynucleotides comprising SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively, and (b) a second vector or lipoplex (e.g., LNP) comprising first and second polynucleotides comprising SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively. In some embodiments, the kit comprises first, second, third and fourth viral vectors or lipoplexes (e.g., LNPs), each vector or lipoplex (e.g., LNP) comprising first and second polynucleotides: (a) a first viral vector or lipoplex (e.g., LNP) comprising first and second polynucleotides comprising SEQ ID NOs: 131 and 135, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; (b) a second viral vector or lipoplex (e.g., LNP) comprising first and second polynucleotides comprising SEQ ID NOs: 133 and 137, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; (c) a third viral vector or lipoplex (e.g., LNP) comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and (d) a fourth viral vector or lipoplex (e.g., LNP) comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In some embodiments, the kit comprises first and second viral vectors or lipoplexes (e.g., LNPs) comprising the following first polynucleotide and second polynucleotide: (a) a first vector or lipoplex (e.g., LNP) comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively, and (b) a second vector or lipoplex (e.g., LNP) comprising first and second polynucleotides comprising SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively. In some embodiments, the first and second viral vectors are Lymphocytic choriomeningitis mammarenavirus (LCMV) viral vectors. In some embodiments, the kit comprises first and second viral vectors or lipoplexes (e.g., LNPs) comprising the following first polynucleotide and second polynucleotide: (a) a first vector or lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and (b) a second vector or lipoplex (e.g., LNP) comprising first and second polynucleotides comprising SEQ ID NOs: 154 and 157 or SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In some embodiments, the first and second viral vectors are Cali mammarenavirus (a.k.a. Pichinde mammarenavirus or Pichinde arenavirus) viral vectors. In some embodiments, the kit comprises first, second, third and fourth viral vectors or lipoplexes (e.g., LNPs), each vector or lipoplex (e.g., LNP) comprising first and second polynucleotides: (a) a first viral vector or lipoplex (e.g., LNP) comprising first and second polynucleotides comprising SEQ ID NOs: 140 and 146, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; (b) a second viral vector or lipoplex (e.g., LNP) comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; (c) a third viral vector or lipoplex (e.g., LNP) comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and (d) a fourth viral vector or lipoplex (e.g., LNP) comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In some embodiments, the kit comprises first, second, third and fourth viral vectors or lipoplexes (e.g., LNPs), each vector or lipoplex (e.g., LNP) comprising first and second polynucleotides: (a) a first viral vector or lipoplex (e.g., LNP) comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; (b) a second viral vector or lipoplex (e.g., LNP) comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; (c) a third viral vector or lipoplex (e.g., LNP) comprising first and second polynucleotides comprising SEQ ID NOs: 151 and 153, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; and (d) a fourth viral vector or lipoplex (e.g., LNP) comprising first and second polynucleotides comprising SEQ ID NOs: 156 and 159, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively. In various embodiments, one or more of the first, second, third and fourth vectors are adenoviral vectors. In some embodiments, the kit comprises first and second viral vectors or lipoplexes (e.g., LNPs) comprising the following first polynucleotide and second polynucleotide: (a) a first vector or lipoplex (e.g., LNP) comprising first and second polynucleotides comprising SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively, and (b) a second vector or lipoplex (e.g., LNP) comprising first and second polynucleotides comprising SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively. In some embodiments, the kit comprises first and second viral vectors comprising the following first polynucleotide and second polynucleotide: (a) a first vector or lipoplex (e.g., LNP) comprising first and second polynucleotides comprising SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively, and (b) a second vector or lipoplex (e.g., LNP) comprising first and second polynucleotides comprising SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively. In some embodiments, the kit comprises (a) a first vector or lipoplex (e.g., LNP) comprising a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and (b) a second vector or lipoplex (e.g., LNP) comprising a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211. In some embodiments, the kit comprises (a) a first vector or lipoplex (e.g., LNP) comprising a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and (b) a second vector or lipoplex (e.g., LNP) comprising a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213. In some embodiments, the kit comprises (a) a first vector or lipoplex (e.g., LNP) comprising a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and (b) a second vector or lipoplex (e.g., LNP) comprising a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215. In some embodiments, the kit comprises (a) a first vector or lipoplex (e.g., LNP) comprising a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and (b) a second vector or lipoplex (e.g., LNP) comprising a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217. In some embodiments, the kit comprises (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219. In some embodiments, the kit comprises (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the kit comprises (a) a first vector or lipoplex (e.g., LNP) comprising a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the kit comprises a compound fusion polypeptide, a vector, or a lipoplex (e.g., LNP) comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the kit comprises a compound fusion polypeptide, a vector, or a lipoplex (e.g., LNP) comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In some embodiments, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof. In some embodiments, the kit further comprises one or more unitary doses of one or more additional therapeutic agents. In some embodiments, the kit comprises one or more agents that activate latent HIV, e.g., one or more latency reversing agents (LRAs). In some embodiments, the kit comprises one or more LRAs selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the kit comprises one or more agonists or activators of one or more toll-like receptors (TLRs). In some embodiments, the TLR agonist or activator is selected from a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from GS 9620 (vesatolimod), R848 (Resiquimod), DS-0509, LHC-165 and TMX-101 (imiquimod), and/or wherein the TLR8 agonist is selected from GS-9688 (Selgantolimod), R848 (Resiquimod) and NKTR-262 (dual TLR7/TLR8 agonist). In some embodiments, the kit comprises one or more interleukin receptor agonists of an interleukin selected from IL-2, IL-7, IL-12 and IL-15. In some embodiments, the kit comprises one or more cytokines selected from IL-2, IL-7, IL-12, IL-15, and variants thereof. In some embodiments, the kit comprises one or more innate immune activators. In some embodiments, the one or more innate immune activators comprises an agonist of a receptor selected from fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, the kit comprises an agonist of fms related tyrosine kinase 3 (FLT3). In some embodiments, the kit comprises one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCNI, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL 16 binding protein 1 (ULBP1); UL16 binding protein 2 (ULBP2); UL 16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor DI (KLRD1); and SLAM family member 7 (SLAMF7). In some embodiments, the kit comprises one or more blockers or inhibitors of one or more T-cell inhibitory immune checkpoint proteins or receptors. In some embodiments, the T-cell inhibitory immune checkpoint proteins or receptors are selected from CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the kit comprises one or more agonists or activators of one or more T-cell stimulatory immune checkpoint proteins or receptors. In some embodiments the T-cell stimulatory immune checkpoint proteins or receptors are selected from CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the kit comprises one or more blockers or inhibitors of one or more NK-cell inhibitory immune checkpoint proteins or receptors. In some embodiments, the NK-cell inhibitory immune checkpoint proteins or receptors are selected from killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor DI (KLRD1, CD94). In some embodiments, the kit comprises one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous inhibitor of PD-L1 (CD274), PD-1 (PDCD1) or CTLA4. In some embodiments, the kit comprises and antibody that binds to CTLA4. In some embodiments, the proteinaceous or antibody inhibitor of CTLA4 is selected from ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous inhibitor of PD-L1 (CD274) or PD-1 (PDCD1) is selected from pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCDI is selected from GS-4224, GS-4416, INCB086550 and MAX10181. In some embodiments, the small molecule inhibitor of CTLA4 comprises BPI-002. In some embodiments, the kit further comprises one or more antiviral agents. In some embodiments, the one or more antiviral agents are selected from HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

Further provided are methods for eliciting an immune response to human immunodeficiency virus (HIV) in a subject in need thereof, comprising administering to the subject a fusion polypeptide, a compound fusion polypeptide, a polynucleotide, a vector, a lipoplex (e.g., LNP) or an immunogenic composition, as described herein. Also provided are methods of treating or preventing human immunodeficiency virus (HIV) in a subject in need thereof, comprising administering to the subject a fusion polypeptide, a compound fusion polypeptide, a vector, a lipoplex (e.g., LNP) or an immunogenic composition, as described herein. In some embodiments the method entails administering a single vector comprising one or more polynucleotides encoding a first fusion polypeptide and a second fusion polypeptide. In some embodiments, two or more fusion polypeptides, two or more compound fusion polypeptides, two or more polynucleotides encoding the fusion polypeptides, two or more viral expression vectors comprising polynucleotides encoding the fusion polypeptides, two or more lipoplexes (e.g., LNPs) or two or more immunogenic compositions, as described herein, are administered to the subject simultaneously or concurrently. In some embodiments, two or more fusion polypeptides, or two or more polynucleotides or two or more viral expression vectors encoding the fusion polypeptides, are in the form of a bivalent antigen composition. In some embodiments, the method entails administering a first fusion polypeptide and a second fusion polypeptide, one or more polynucleotides encoding the first and second fusion polypeptides, one or more vectors comprising one or more polynucleotides encoding the first and second fusion polypeptides, or one or more lipoplexes (e.g., LNPs), the first and second polypeptides comprising the following polypeptide segments, in sequential order, from N-terminus to C terminus, optionally joined or connected by one or more linkers: SEQ ID NOs: 6, 4, 16 and 26, and SEQ ID NOs: 7, 5, 27 and 17; SEQ ID NOs: 12, 24, 8 and 10, and SEQ ID NOs: 9, 25, 13 and 11; SEQ ID NOs: 14, 22, 18, 24 and 20, and SEQ ID NOs: 15, 25, 19, 23 and 21; SEQ ID NOs: 26, 16, 4 and 6, and SEQ ID NOs: 7, 27, 5 and 17; SEQ ID NOs: 8, 24, 12 and 10, and SEQ ID NOs: 13, 25, 9 and 11; SEQ ID NOs: 22, 24, 14, 18 and 20, and SEQ ID NOs: 25, 23, 15, 21 and 19; SEQ ID NOs: 20, 6, 4, 18, 16 and 26, and SEQ ID NOs: 7, 19, 5, 21, 27 and 17; SEQ ID NOs: 8, 24, 14, 12, 22 and 10, and SEQ ID NOs: 9, 13, 25, 23, 15 and 11; SEQ ID NOS: 18, 26, 20, 4, 6 and 16, and SEQ ID NOs: 7, 21, 17, 5, 27 and 19; SEQ ID NOs: 22, 24, 12, 14, 8 and 10, and SEQ ID NOs: 15, 25, 9, 23, 13 and 11; SEQ ID NOs: 24, 6, 4, 20, 18 and 32, and SEQ ID NOs: 8, 30, 14, 12, 26 and 10; SEQ ID NOs: 24, 6, 4, 20, 18 and 32, and SEQ ID NOs: 7, 21, 5, 25, 33 and 19; SEQ ID NOs: 24, 6, 4, 20, 18 and 32, and SEQ ID NOs: 8, 30, 14, 12, 26 and 10; SEQ ID NOs: 8, 30, 14, 12, 26 and 10, and SEQ ID NOs: 9, 13, 31, 27, 15 and 11; SEQ ID NOs: 6, 20, 4, 24, 32 and 18, and SEQ ID NOs: 8, 12, 30, 26, 14 and 10; SEQ ID NOs: 7, 21, 5, 25, 33 and 19 and SEQ ID NOs: 9, 13, 31, 27, 15 and 11; SEQ ID NOs: 20, 32, 24, 4, 6 and 18, and SEQ ID NOs: 26, 30, 12, 14, 8 and 10; SEQ ID NOs: 7, 25, 19, 5, 33 and 21, and SEQ ID NOs: 15, 31, 9, 27, 13 and 11; SEQ ID NOs: 24, 6, 16 and 18, and SEQ ID NOs: 26, 20, 10 and 28; SEQ ID NOs: 24, 6, 16 and 18, and SEQ ID NOs: 26, 10, 20 and 28; SEQ ID NOs: 24, 6, 16 and 18, and SEQ ID NOs: 7, 25, 17 and 19; SEQ ID NOs: 7, 19, 17 and 25, and SEQ ID NOs: 21, 27, 11 and 29; SEQ ID NOs: 24, 16, 6 and 18, and SEQ ID NOs: 27, 11, 21 and 29; SEQ ID NOs: 7, 25, 17 and 19, and SEQ ID NOs: 11, 27, 21 and 29; SEQ ID NOs: 7, 25, 17 and 19, and SEQ ID NOs: 21, 27, 11 and 29; SEQ ID NOs: 22, 6, 20 and 28, and SEQ ID NOs: 23, 7, 21 and 29; SEQ ID NOs: 22, 20, 6 and 28, and SEQ ID NOs: 7, 21, 23 and 29; SEQ ID NOs: 26, 10, 20 and 28, and SEQ ID NOs: 21, 27, 11 and 29; SEQ ID NOs: 22, 6, 16, 20, 18, 28, 26 and 10; or SEQ ID NOs: 7, 21, 19, 17, 27, 25, 29 and 11. In some embodiments, the method entails administering one or more fusion polypeptides, one or more polynucleotides encoding one or more fusion polypeptides, one or more vectors comprising one or more polynucleotides encoding one or more fusion polypeptides, one or more lipoplexes (e.g., LNPs), the one or more fusion polypeptides comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the method entails administering one or more fusion polypeptides, one or more polynucleotides encoding one or more fusion polypeptides, one or more vectors comprising one or more polynucleotides encoding one or more fusion polypeptides, one or more lipoplexes (e.g., LNPs), the one or more fusion polypeptides comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223. In some embodiments, the method entails administering the following first fusion polypeptide and second fusion polypeptide, one or more polynucleotides, one or more vectors or one or more lipoplexes (e.g., LNPs), comprising one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the method entails administering the following first fusion polypeptide and second fusion polypeptide, one or more vectors, or one or more lipoplexes (e.g., LNPs), comprising one or more polynucleotides encoding the following first fusion polypeptide and second fusion polypeptide, in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively; SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively, and (b) a second polynucleotide or second viral vector or second lipoplex (e.g., LNP) comprising one or more polynucleotides encoding the following first fusion polypeptide and second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively, and (b) a second polynucleotide or second viral vector or second lipoplex (e.g., LNP) comprising one or more polynucleotides encoding the following first fusion polypeptide and second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively, and (b) a second polynucleotide or second viral vector or second lipoplex (e.g., LNP) comprising one or more polynucleotides encoding the following first fusion polypeptide and second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively, and (b) a second polynucleotide or second viral vector or second lipoplex (e.g., LNP) comprising one or more polynucleotides encoding the following first fusion polypeptide and second fusion polypeptide comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively, and (b) a second polynucleotide or second viral vector or second lipoplex (e.g., LNP) comprising one or more polynucleotides encoding the following first fusion polypeptide and second fusion polypeptide comprising SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively, and (b) a second polynucleotide or second viral vector or second lipoplex (e.g., LNP) comprising one or more polynucleotides encoding the following first fusion polypeptide and second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively, and (b) a second polynucleotide or second viral vector or second lipoplex (e.g., LNP) comprising one or more polynucleotides encoding the following first fusion polypeptide and second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and (b) a second polynucleotide or second viral vector or second lipoplex (e.g., LNP) comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and (b) a second polynucleotide or second viral vector or second lipoplex (e.g., LNP) comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second polynucleotide or second viral vector or second lipoplex (e.g., LNP) comprising one or more polynucleotides encoding the following first fusion polypeptide and second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the method entails administering first and second viral vectors or first and second lipoplexes (e.g., LNPs) comprising one or more polynucleotides encoding the following first fusion polypeptide and second fusion polypeptide, optionally joined or connected by one or more linkers: (a) a first polynucleotide or first viral vector or first lipoplex (e.g., LNP) comprising one or more polynucleotides encoding the following first fusion polypeptide and second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second polynucleotide or second viral vector or second lipoplex (e.g., LNP) comprising one or more polynucleotides encoding the following first fusion polypeptide and second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 200, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, and (b) a second polynucleotide or second viral vector or second lipoplex (e.g., LNP) comprising a polynucleotide encoding a second fusion polypeptide comprising SEQ ID NO: 201, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 202, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202, and (b) a second polynucleotide or second viral vector or second lipoplex (e.g., LNP) comprising a polynucleotide encoding a second fusion polypeptide comprising SEQ ID NO: 203, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 204, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204, and (b) a second polynucleotide or second viral vector or second lipoplex (e.g., LNP) comprising a polynucleotide encoding a second fusion polypeptide comprising SEQ ID NO: 205, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 105, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, and (b) a second polynucleotide or second viral vector or second lipoplex (e.g., LNP) comprising a polynucleotide encoding a second fusion polypeptide comprising SEQ ID NO: 107, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 206, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, and (b) a second polynucleotide or second viral vector or second lipoplex (e.g., LNP) comprising a polynucleotide encoding a second fusion polypeptide comprising SEQ ID NO: 207, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 208, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, and (b) a second polynucleotide or second viral vector or second lipoplex (e.g., LNP) comprising a polynucleotide encoding a second fusion polypeptide comprising SEQ ID NO: 209 or SEQ ID NO: 227, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 222, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second polynucleotide or second viral vector or second lipoplex (e.g., LNP) comprising a polynucleotide encoding a second fusion polypeptide comprising SEQ ID NO: 209 or SEQ ID NO: 227, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227. In some embodiments, the method entails administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 222, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second polynucleotide or second viral vector or second lipoplex (e.g., LNP) comprising a polynucleotide encoding a second fusion polypeptide comprising SEQ ID NO: 223, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In some embodiments, the method entails administering a viral vector or a lipoplex (e.g., LNP) comprising one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively. In some embodiments, the method entails administering a viral vector or a lipoplex (e.g., LNP) comprising one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively. In various embodiments, the first and second viral vectors are Lymphocytic choriomeningitis mammarenavirus (LCMV) viral vectors, Cali mammarenavirus (a.k.a. Pichinde mammarenavirus or Pichinde arenavirus) viral vectors or adenoviral vectors, e.g., chimpanzee adenoviral vectors (ChAds). In some embodiments, the first and second viral vectors or first and second lipoplexes (e.g., LNPs) are co-administered concurrently. In some embodiments, the methods entail administering the following first polynucleotide and second polynucleotide, one or more vectors, or one or more lipoplexes (e.g., LNPs) comprising the following first polynucleotide and second polynucleotide: SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively; SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively; SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively; SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively; SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively; SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively; SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively; SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively; SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively; SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively; SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively; SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively; SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively; SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively; SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively; SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively; SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively; SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively; SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively; SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively; SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively; SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively; SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively; SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively; SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively; SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively. In some embodiments, the method entails administering first and second viral vectors, or first and second lipoplexes (e.g., LNPs), comprising the following first polynucleotide and second polynucleotide: (a) a first vector or lipoplex (e.g., LNP) comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively, and (b) a second vector or lipoplex (e.g., LNP) comprising first and second polynucleotides comprising SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively, wherein the first and second viral vectors are Lymphocytic choriomeningitis mammarenavirus (LCMV) viral vectors. In some embodiments, the method entails administering first and second viral vectors, or first and second lipoplexes (e.g., LNPs), comprising the following first polynucleotide and second polynucleotide: (a) a first vector or lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and (b) a second vector or lipoplex (e.g., LNP) comprising first and second polynucleotides comprising SEQ ID NOs: 154 and 157 or SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively, wherein the first and second viral vectors are Cali mammarenavirus (a.k.a. Pichinde mammarenavirus or Pichinde arenavirus) viral vectors. In some embodiments, the method entails administering a compound fusion polypeptide, a vector, or a lipoplex (e.g., LNP) comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the method entails administering a compound fusion polypeptide, a vector, or a lipoplex (e.g., LNP) comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the methods entail administering a polynucleotide (e.g., in a vector, in a lipoplex (e.g., LNP)) comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the methods entail administering a polynucleotide (e.g., in a vector, in a lipoplex (e.g., LNP)) comprising or consisting of a nucleic acid sequence of any one of SEQ ID NOs: 139, 140, 142, 143, 145, 146, 148, 149, 150, 152, 155, 158, 225 and 226, or a nucleic sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226. In some embodiments, the method entails administering: (a) a first vector or lipoplex (e.g., LNP) comprising a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and (b) a second vector or lipoplex (e.g., LNP) comprising a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 211. In some embodiments, the method entails administering: (a) a first vector or lipoplex (e.g., LNP) comprising a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and (b) a second vector or lipoplex (e.g., LNP) comprising a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 213. In some embodiments, the method entails administering: (a) a first vector or lipoplex (e.g., LNP) comprising a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and (b) a second vector or lipoplex (e.g., LNP) comprising a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 215. In some embodiments, the method entails administering: (a) a first vector or lipoplex (e.g., LNP) comprising a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and (b) a second vector or lipoplex (e.g., LNP) comprising a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 217. In some embodiments, the method entails administering: (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219. In some embodiments, the method entails administering: (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the method entails administering: (a) a first vector or lipoplex (e.g., LNP) comprising a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In some embodiments, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, the fusion polypeptide, the compound fusion polypeptide, the polynucleotide, the vector, the lipoplex (e.g., LNP) or the immunogenic composition is administered via a route selected from intravenous, intramuscular, intradermal, subcutaneous, intranodal and mucosal (e.g. buccal, intranasal, intrarectal, intravaginal). In some embodiments, the method entails administering from about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the methods comprise a prime-boost regimen comprising administering a priming composition at a first time point and administering one or more boosting compositions at one or more subsequent time points. In some embodiments, the methods comprise repeating the prime-boost regimen one or more iterations. In some embodiments, the administrations of the priming composition and the one or more boosting compositions are spaced at least 1 week, 2 weeks, 3 weeks or 1 month apart, e.g., at least 2, 3, 4, 5, 6, 7, 8, 9, 10, 11 or 12 months apart. In some embodiments, the priming composition and the boosting composition comprise the same immunogenic composition. In some embodiments, the priming composition and the boosting composition comprise different immunogenic compositions. In some embodiments, the priming composition and the boosting composition comprise the same one or more fusion polypeptides and same viral expression vector. In some embodiments, the priming composition and the boosting composition comprise different fusion polypeptides and/or different viral expression vectors. In some embodiments, the methods comprise priming with a first viral expression vector, and boosting with a second viral expression vector. In some embodiments, the prime-boost regimen comprises: (a) Priming with one or more viral expression vectors and boosting with one or more polynucleotides, wherein the one or more polynucleotides comprise DNA, cDNA, mRNA or self-replicating RNA; (b) Priming with one or more polynucleotides, wherein the one or more polynucleotides comprise DNA, cDNA, mRNA or self-replicating RNA, and boosting with one or more viral expression vectors; (c) Priming with one or more viral expression vectors, and boosting with one or more viral expression vectors, wherein the one or more viral expression vectors in the priming composition and the one or more viral expression vectors in the boosting composition are from identical, related or unrelated taxonomical families; (d) Priming with one or more replication-deficient viral expression vectors and boosting with one or more replication-deficient viral expression vectors, wherein the one or more replication-deficient viral expression vectors in the priming composition and the one or more replication-deficient viral expression vectors in the boosting composition are from identical, related or unrelated taxonomical families; (e) Priming with one or more replication-attenuated viral expression vectors and boosting with one or more replication-attenuated viral expression vectors, wherein the one or more replication-attenuated viral expression vectors in the priming composition and the one or more replication-attenuated viral expression vectors in the boosting composition are from identical, related or unrelated taxonomical families; (f) Priming with one or more replication-deficient viral expression vectors and boosting with one or more replication-attenuated viral expression vectors; (g) Priming with one or more replication-attenuated viral expression vectors and boosting with one or more replication-deficient viral expression vectors; (h) Priming with one or more Lymphocytic choriomeningitis mammarenavirus (LCMV) viral expression vectors and boosting with one or more Pichinde mammarenavirus viral expression vectors; (i) Priming with one or more Pichinde mammarenavirus viral expression vectors and boosting with one or more Lymphocytic choriomeningitis mammarenavirus (LCMV) viral expression vectors; (j) Priming with one or more replication deficient Pichinde mammarenavirus viral expression vectors and boosting with one or more replication deficient Lymphocytic choriomeningitis mammarenavirus (LCMV) viral expression vectors; (k) Priming with one or more replication deficient Lymphocytic choriomeningitis mammarenavirus (LCMV) viral expression vectors and boosting with one or more replication deficient Pichinde mammarenavirus viral expression vectors; (1) Priming with one or more arenavirus viral expression vectors and boosting with one or more adenovirus viral expression vectors; (m) Priming with one or more adenovirus viral expression vectors and boosting with boosting composition comprising one or more arenavirus viral expression vectors; (n) Priming with one or more adenovirus viral expression vectors and boosting with boosting composition comprising one or more RNA molecules (e.g., mRNA, self-amplifying or self-replicating RNA); (o) Priming with one or more RNA molecules (e.g., mRNA, self-amplifying or self-replicating RNA) and boosting with boosting composition comprising one or more adenovirus viral expression vectors; (p) Priming with one or more chimpanzee adenoviral (ChAd) expression vectors and boosting with boosting composition comprising one or more self-amplifying or self-replicating RNA (saRNA or samRNA); (q) Priming with one or more self-amplifying or self-replicating RNA (saRNA or samRNA) and boosting with boosting composition comprising one or more chimpanzee adenoviral (ChAd) expression vectors; (r) Priming with one or more poxvirus viral expression vectors and boosting with one or more arenavirus viral expression vectors; (s) Priming with one or more arenavirus viral expression vectors and boosting with boosting composition comprising one or more poxvirus viral expression vectors; (t) Priming with one or more poxvirus viral expression vectors and boosting with one or more adenovirus viral expression vectors; or (u) Priming with one or more adenovirus viral expression vectors and boosting with boosting composition comprising one or more poxvirus viral expression vectors. In some embodiments, the prime-boost regimen comprises: (1) Priming with an immunogenic composition comprising the following first fusion polypeptide and second fusion polypeptide, one or more vectors, or one or more lipoplexes (e.g., LNPs) comprising one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; and (2) Boosting with an immunogenic composition comprising the following first fusion polypeptide and second fusion polypeptide, one or more vectors, or one or more lipoplexes (e.g., LNPs) comprising one or more polynucleotides encoding the following first fusion polypeptide and second fusion polypeptide, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively; SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively; SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively; SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively; SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively; SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively; SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; SEQ ID NOS: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively; SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively; SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively; SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively; SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the prime-boost regimen comprises: (1) Priming with an immunogenic composition comprising the following first fusion polypeptide and second fusion polypeptide, one or more vectors, one or more lipoplexes (e.g., LNPs), comprising one or more polynucleotides encoding the following first fusion polypeptide and second fusion polypeptide, optionally joined or connected by one or more linkers: (a) SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; (b) SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; (c) SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; (d) SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and/or (e) SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; and (2) Boosting with an immunogenic composition comprising the following first fusion polypeptide and second fusion polypeptide, one or more vectors, or one or more lipoplexes (e.g., LNPs), comprising one or more polynucleotides encoding the following first fusion polypeptide and second fusion polypeptide, optionally joined or connected by one or more linkers: (a) SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; (b) SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; (c) SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and/or (d) SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively or one or more polynucleotides encoding first fusion polypeptide and second fusion polypeptides comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and (b) a second viral vector or lipoplex (e.g., LNP) comprising one or more polynucleotides encoding first fusion polypeptide and second fusion polypeptides comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (b) a second viral vector or lipoplex (e.g., LNP) comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively. In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively. In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 105, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, respectively; and (2) Boosting with an immunogenic composition comprising a viral vector or a lipoplex (e.g., LNP) comprising a polynucleotide encoding a compound fusion polypeptide comprising SEQ ID NO: 109, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 109, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 105 or 206, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105 or 206, respectively; and (2) Boosting with an immunogenic composition comprising a viral vector or a lipoplex (e.g., LNP) comprising a polynucleotide encoding a compound fusion polypeptide comprising SEQ ID NO: 107 or 207, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107 or 207, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 105, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, respectively; and a second viral vector or a lipoplex (e.g., LNP) comprising a polynucleotide encoding a second fusion polypeptide, comprising SEQ ID NO: 107, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 206, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, respectively; and a second viral vector or a lipoplex (e.g., LNP) comprising a polynucleotide encoding a second fusion polypeptide, comprising SEQ ID NO: 207, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 208, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, respectively; and (2) Boosting with an immunogenic composition comprising a viral vector or a lipoplex (e.g., LNP) comprising a polynucleotide encoding a compound fusion polypeptide comprising SEQ ID NO: 209 or SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and (2) Boosting with an immunogenic composition comprising a viral vector or a lipoplex (e.g., LNP) comprising a polynucleotide encoding a compound fusion polypeptide comprising SEQ ID NO: 209 or SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and (2) Boosting with an immunogenic composition comprising a viral vector or a lipoplex (e.g., LNP) comprising a polynucleotide encoding a compound fusion polypeptide comprising SEQ ID NO: 223, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 208, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, respectively; and a second viral vector or a lipoplex (e.g., LNP) comprising a polynucleotide encoding a second fusion polypeptide, comprising SEQ ID NO: 209 or SEQ ID NO: 227, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 222, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and a second viral vector or a lipoplex (e.g., LNP) comprising a polynucleotide encoding a second fusion polypeptide, comprising SEQ ID NO: 209 or SEQ ID NO: 227, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 222, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and a second viral vector or a lipoplex (e.g., LNP) comprising a polynucleotide encoding a second fusion polypeptide, comprising SEQ ID NO: 223, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223, respectively. In some embodiments, the prime-boost regimen comprises: (1) Priming with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; and (2) Boosting with an immunogenic composition comprising a viral vector or a lipoplex (e.g., LNP) comprising one or more polynucleotides encoding first fusion polypeptide and second fusion polypeptides, optionally joined or connected by one or more linkers, comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 107, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107, respectively; and (2) Boosting with an immunogenic composition comprising a viral vector or a lipoplex (e.g., LNP) comprising a polynucleotide encoding a compound fusion polypeptide comprising SEQ ID NO: 111, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 111, respectively. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 200, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, respectively; and (2) Boosting with an immunogenic composition comprising a viral vector or a lipoplex (e.g., LNP) comprising a polynucleotide encoding a compound fusion polypeptide comprising SEQ ID NO: 201, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201, respectively. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 200, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200; and a second viral vector or a lipoplex (e.g., LNP) comprising a polynucleotide encoding a second fusion polypeptide, comprising SEQ ID NO: 201, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 202, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202; and (2) Boosting with an immunogenic composition comprising a viral vector or a lipoplex (e.g., LNP) comprising a polynucleotide encoding a compound fusion polypeptide comprising SEQ ID NO: 203, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 202, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202; and a second viral vector or a lipoplex (e.g., LNP) comprising a polynucleotide encoding a second fusion polypeptide, comprising SEQ ID NO: 203, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203. In some embodiments, the prime-boost regimen comprises: (1) Priming with an immunogenic composition comprising a viral vector or a lipoplex (e.g., LNP) comprising a polynucleotide encoding a compound fusion polypeptide comprising SEQ ID NO: 204, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204; and (2) Boosting with an immunogenic composition comprising a viral vector or a lipoplex (e.g., LNP) comprising a polynucleotide encoding a compound fusion polypeptide comprising SEQ ID NO: 205, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205. In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 204, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204; and a second viral vector or a lipoplex (e.g., LNP) comprising a polynucleotide encoding a second fusion polypeptide, comprising SEQ ID NO: 205, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; and (b) a second viral vector or lipoplex (e.g., LNP) comprising one or more polynucleotides encoding first fusion polypeptide and second fusion polypeptides comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; and (b) a second viral vector or lipoplex (e.g., LNP) comprising one or more polynucleotides encoding first fusion polypeptide and second fusion polypeptides comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and (b) a second viral vector or lipoplex (e.g., LNP) comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector comprising one or more polynucleotides encoding first fusion polypeptide and second fusion polypeptides comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; and (b) a second viral vector comprising one or more polynucleotides encoding first fusion polypeptide and second fusion polypeptides comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and (b) a second viral vector or lipoplex (e.g., LNP) comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (b) a second viral vector or lipoplex (e.g., LNP) comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and (b) a second viral vector or lipoplex (e.g., LNP) comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (b) a second viral vector or lipoplex (e.g., LNP) comprising one or more polynucleotides encoding first fusion polypeptide and second fusion polypeptides comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (b) a second viral vector or lipoplex (e.g., LNP) comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and (b) a second viral vector or lipoplex (e.g., LNP) comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the prime-boost regimen comprises: (1) Priming with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and (b) a second viral vector or lipoplex (e.g., LNP) comprising one or more polynucleotides encoding first fusion polypeptide and second fusion polypeptides comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; and (2) Boosting with an immunogenic composition comprising first and second viral vectors, or first and second lipoplexes (e.g., LNPs), comprising one or more polynucleotides encoding the following first fusion polypeptide and second fusion polypeptide, optionally joined or connected by one or more linkers: (a) a first viral vector or lipoplex (e.g., LNP) comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and (b) a second viral vector or lipoplex (e.g., LNP) comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In some embodiments, the prime-boost regimen comprises: (1) Priming with an immunogenic composition comprising a viral vector or lipoplex (e.g., LNP) comprising one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; and (2) Boosting with an immunogenic composition comprising a viral vector or lipoplex (e.g., LNP) comprising one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively. In some embodiments, the prime-boost regimen comprises: (1) Priming with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; and (b) a second viral vector or lipoplex (e.g., LNP) comprising first and second polynucleotides comprising SEQ ID NOs: 133 and 137, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; and (2) Boosting with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and (b) a second viral vector or lipoplex (e.g., LNP) comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In some embodiments, the prime-boost regimen comprises: (1) Priming with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; and (b) a second viral vector or lipoplex (e.g., LNP) comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; and (2) Boosting with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and (b) a second viral vector or lipoplex (e.g., LNP) comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In some embodiments, the prime-boost regimen comprises: (1) Priming with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and (b) a second viral vector or lipoplex (e.g., LNP) comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively; and (2) Boosting with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 140 and 146, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; and (b) a second viral vector or lipoplex (e.g., LNP) comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively. In some embodiments, the prime-boost regimen comprises: (1) Priming with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and (b) a second viral vector or lipoplex (e.g., LNP) comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; and (2) Boosting with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 151 and 153, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; and (b) a second viral vector or lipoplex (e.g., LNP) comprising first and second polynucleotides comprising SEQ ID NOs: 156 and 159, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively. In some embodiments, the prime-boost regimen comprises: (1) Priming with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and (b) a second viral vector or lipoplex (e.g., LNP) comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; and (2) Boosting with an immunogenic composition comprising first and second viral vectors, or first and second lipoplexes (e.g., LNPs), comprising the following polynucleotides: (a) a first viral vector or lipoplex (e.g., LNP) comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145 respectively; and (b) a second viral vector or lipoplex (e.g., LNP) comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In some embodiments, the prime-boost regimen comprises: (1) Priming with an immunogenic composition comprising a first viral vector or lipoplex (e.g., LNP) comprising first and second polynucleotides comprising SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; and (2) Boosting with an immunogenic composition comprising a second viral vector or lipoplex (e.g., LNP) comprising first and second polynucleotides comprising SEQ ID NOs: 162 and 163, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively. In some embodiments, the prime-boost regimen comprises: (1) Priming with an immunogenic composition comprising a first viral vector or lipoplex (e.g., LNP) comprising first and second polynucleotides comprising SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; and (2) Boosting with an immunogenic composition comprising a second viral vector or lipoplex (e.g., LNP) comprising first and second polynucleotides comprising SEQ ID NOs: 166 and 167, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively. In some embodiments, the viral vectors in the priming composition are Lymphocytic choriomeningitis mammarenavirus (LCMV) viral vectors and the viral vectors in the boosting composition are Cali mammarenavirus (a.k.a. Pichinde mammarenavirus or Pichinde arenavirus) viral vectors. In various embodiments, the viral vectors in the priming composition are Cali mammarenavirus (a.k.a. Pichinde mammarenavirus or Pichinde arenavirus) viral vectors and the viral vectors in the boosting composition are Lymphocytic choriomeningitis mammarenavirus (LCMV) viral vectors. In some embodiments, the prime-boost regimen comprises: priming and boosting with an immunogenic composition comprising a first viral vector or lipoplex (e.g., LNP) comprising a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210; and a second viral vector or lipoplex (e.g., LNP) comprising a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211. In some embodiments, the prime-boost regimen comprises: priming and boosting with an immunogenic composition comprising a first viral vector or lipoplex (e.g., LNP) comprising a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212; and a second viral vector or lipoplex (e.g., LNP) comprising a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213. In some embodiments, the prime-boost regimen comprises: priming and boosting with an immunogenic composition comprising a first viral vector or lipoplex (e.g., LNP) comprising a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214; and a second viral vector or lipoplex (e.g., LNP) comprising a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215. In some embodiments, the prime-boost regimen comprises: priming and boosting with an immunogenic composition comprising a first viral vector or lipoplex (e.g., LNP) comprising a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216; and a second viral vector or lipoplex (e.g., LNP) comprising a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217. In some embodiments, the prime-boost regimen comprises: priming and boosting with an immunogenic composition comprising a first viral vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218; and a second viral vector or lipoplex (e.g., LNP) comprising a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219. In some embodiments, the prime-boost regimen comprises: priming and boosting with an immunogenic composition comprising a first viral vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218; and a second viral vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the prime-boost regimen comprises: priming and boosting with an immunogenic composition comprising a first viral vector or lipoplex (e.g., LNP) comprising a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225; and a second viral vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In some embodiments, the viral vector(s) in the priming composition and the viral vector(s) in the boosting composition are adenoviral vectors, e.g., chimpanzee adenoviral vectors (ChAds). In some embodiments, the viral vectors in the priming composition and the viral vectors in the boosting composition are replication deficient. In some embodiments, the viral vectors in the priming composition and the viral vectors in the boosting composition are replication attenuated. In some embodiments, the subject is not receiving antiretroviral therapy (ART) or ART is discontinued prior to administration of the one or more compositions. In some embodiments, ART is discontinued after one or more administrations of the compositions. In some embodiments, the methods entail administering to the subject one or more additional therapeutic agents, e.g. two, three, four, or more additional therapeutic agents. In some embodiments, the methods entail co-administering one or more agents that activate latent HIV, e.g., one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the methods entail co-administering one or more agonists or activators of one or more toll-like receptors (TLRs). In some embodiments, the TLR agonist or activator is selected from a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from GS 9620 (vesatolimod), R848 (Resiquimod), DS-0509, LHC-165 and TMX-101 (imiquimod), and/or wherein the TLR8 agonist is selected from GS-9688 (Selgantolimod), R848 (Resiquimod) and NKTR-262 (dual TLR7/TLR8 agonist). In some embodiments, the methods entail co-administering one or more interleukin receptor agonists of an interleukin selected from IL-2, IL-7, IL-12 and IL-15. In some embodiments, the methods entail co-administering one or more cytokines selected from IL-2, IL-7, IL-12, IL-15, and variants thereof. In some embodiments, the methods entail co-administering one or more innate immune activators. In some embodiments, the one or more innate immune activators comprises an agonist of a receptor selected from fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, the methods entail co-administering an agonist of fms related tyrosine kinase 3 (FLT3). In some embodiments, the methods entail co-administering one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL 16 binding protein 1 (ULBP1); UL16 binding protein 2 (ULBP2); UL 16 binding protein 3 (ULBP3); retinoic acid early transcript 1E (RAETIE; ULBP4); retinoic acid early transcript 1G (RAET1G; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7). In some embodiments, the methods entail co-administering one or more blockers or inhibitors of one or more T-cell inhibitory immune checkpoint proteins or receptors. In some embodiments, the T-cell inhibitory immune checkpoint proteins or receptors are selected from CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the methods entail co-administering one or more agonists or activators of one or more T-cell stimulatory immune checkpoint proteins or receptors. In some embodiments, the T-cell stimulatory immune checkpoint proteins or receptors are selected from CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the methods entail co-administering one or more blockers or inhibitors of one or more NK-cell inhibitory immune checkpoint proteins or receptors. In some embodiments, the NK-cell inhibitory immune checkpoint proteins or receptors are selected from killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In some embodiments, the methods entail co-administering one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous inhibitor of PD-L1 (CD274), PD-1 (PDCD1) or CTLA4. In some embodiments, the methods entail co-administering an antibody that binds to CTLA4. In some embodiments, the proteinaceous or antibody inhibitor of CTLA4 is selected from ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous inhibitor of PD-L1 (CD274) or PD-1 (PDCD1) is selected from pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCDI is selected from GS-4224, GS-4416, INCB086550 and MAX10181. In some embodiments, the small molecule inhibitor of CTLA4 comprises BPI-002. In some embodiments, the methods further comprise administering to the subject one or more antiviral agents. In some embodiments, the one or more antiviral agents are selected from HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, the methods further comprise administering to the subject one or more anti-HIV antibodies or antigen-binding fragments thereof. In some embodiments, the one or more anti-HIV antibodies or antigen-binding fragments thereof binds to HIV gp120. In some embodiments, the anti-HIV antibody or antigen-binding fragment thereof comprises a broadly neutralizing antibody. In some embodiments, the one or more anti-HIV antibodies or antigen-binding fragments thereof that bind, inhibit, and/or neutralize HIV, compete with or comprise VH and VL variable domains of a broadly neutralizing antibody (bNAb) against HIV. In some embodiments, one or more anti-HIV antibodies or antigen-binding fragments thereof that bind, inhibit, and/or neutralize HIV, bind to an epitope or region of gp120 selected from: (i) third variable loop (V3) and/or high mannose patch comprising a N332 oligomannose glycan; (ii) CD4 binding site (CD4bs); (iii) second variable loop (V2) and/or Env trimer apex; (iv) gp120/gp41 interface; or (v) silent face of gp120. In some embodiments, the antibody or antigen-binding fragment thereof that binds, inhibits, and/or neutralizes HIV, binds to an epitope or region of gp120 in the third variable loop (V3) and/or high mannose patch comprising a N332 oligomannose glycan and competes with or comprises VH and VL regions from an antibody selected from GS-9722, PGT-121, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, GS-2872, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the antibody or antigen-binding fragment thereof binds to an epitope or region of gp120 in the CD4 binding site (CD4bs) and competes with or comprises VH and VL regions from an antibody selected from b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N49-P7, NC-Cow1, IOMA, CH235 and CH235.12, N49P6, N49P7, N49P11, N49P9 and N60P25. In some embodiments, the antibody or antigen-binding fragment thereof that binds, inhibits, and/or neutralizes HIV, binds to an epitope or region of gp120 in the second variable loop (V2) and/or Env trimer apex and competes with or comprises VH and VL regions from an antibody selected from PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the antibody or antigen-binding fragment binds to an epitope or region of gp120 in the gp120/gp41 interface and competes with or comprises VH and VL regions from an antibody selected from PGT-151, CAP248-2B, 35022, 8ANC195, ACS202, VRC34 and VRC34.01. In some embodiments, the antibody or antigen-binding fragment thereof that binds, inhibits, and/or neutralizes HIV, binds to an epitope or region of the gp120 silent face and competes with or comprises VH and VL regions from antibody selected from VRC-PG05 and SF12. In some embodiments, the antibody or antigen-binding fragment thereof that binds, inhibits, and/or neutralizes HIV, binds to an epitope or region of gp41 in the membrane proximal region (MPER). In some embodiments, the antibody or antigen-binding fragment thereof that binds, inhibits, and/or neutralizes HIV, binds to an epitope or region of gp41 in the membrane proximal region (MPER) and competes with or comprises VH and VL regions from an antibody selected from 10E8, 10E8v4, 10E8-5R-100cF, 4E10, DH511.11P, 2F5, 762, and LN01. In some embodiments, the antibody or antigen-binding fragment thereof that binds, inhibits, and/or neutralizes HIV, binds to an epitope or region of the gp41 fusion peptide and competes with or comprises VH and VL regions from an antibody selected from VRC34 and ACS202. In some embodiments of the methods, after one or more administrations of the one or more fusion polypeptides, compound fusion polypeptides, polynucleotides, vectors, lipoplexes (e.g., LNPS) or immunogenic compositions, optionally in combination with one or more additional therapeutic agents, the subject does not exhibit symptoms of HIV or AIDS in the absence of anti-retroviral treatment (ART) for at least 3 months, at least 6 months, at least 1 year, at least 2 years, at least 3 years, or more. In some embodiments of the methods, after one or more administrations of the one or more fusion polypeptides, compound fusion polypeptides, polynucleotides, vectors, lipoplexes (e.g., LNPS) or immunogenic compositions, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 3 months, at least 6 months, at least 1 year, at least 2 years, at least 3 years, or more.

Further provided are methods of designing a fusion polypeptide that is capable of eliciting an immune response against one or more viral target antigens, the method comprising: (a) identifying in silico one or more regions of sequence conservation in a population of polypeptide sequences encoded by a viral gene, the population from an interpatient virus population; (b) identifying in silico the two most prevalent polypeptide sequences from the one or more conserved regions identified in step a), and generating multivalent polypeptide segments from the conserved regions; and (c) arranging the polypeptide segments to reduce or avoid the creation of deleterious epitopes at junctions between polypeptide segments. In some embodiments, step (c) comprises reducing or eliminating junctional 9-mers that bind to a specific HLA allele with a predicted IC50 value of less than about 1000 nM or having a percentile rank within the top 5% in a population of polypeptide segments. Further provided are methods of designing a fusion polypeptide that is capable of eliciting an immune response against one or more viral target antigens, the method comprising: (a) identifying in silico one or more regions of sequence conservation in a first population of polypeptide sequences encoded by a viral gene, the first population from an interpatient virus population; (b) optionally, identifying in silico the two most prevalent polypeptide sequences from the one or more conserved regions identified in step a); and (c) arranging the retained polypeptide segments into one or more contiguous fusion polypeptides, such that the junctions connecting the polypeptide segments avoid or reduce creating epitopes capable of binding human MHC class I or human MHC class II molecules, e.g., with a predicted binding affinity IC50 value of less than about 1000 nM or having a percentile rank within the top 5% in a population of polypeptide segments. In some embodiments, step (c) comprises reducing or eliminating viral polypeptide 9-mers that have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity to a human protein. In some embodiments, the multivalent polypeptide segments are bivalent polypeptide segments. In some embodiments, the fusion polypeptide design method further comprises the step of identifying variant subsequences within one or more regions of sequence conservation in an intrapatient population of polypeptide sequences encoded by a viral gene, e.g., using deep sequencing data. In some embodiments, the fusion polypeptide design method further comprises the step of identifying conserved regions of a polypeptide encoded by a viral gene, such that at least 70% of the variant subsequences within the one or more regions of sequence conservation in the intrapatient population are within the bivalent polypeptide segments. In some embodiments, the fusion polypeptide design method further comprises the step of shortening the length of the fusion polypeptide, e.g., by at least 10%, 15%, 20%, 25%, 30%, or more, retaining polypeptide segment subsequences comprising epitopes (i) predicted in silico, and (ii) confirmed in vitro. Further provided are methods for producing a multivalent antigen, the method comprising constructing, in silico, a set of multivalent amino acid sequences within structurally conserved regions of a population of viral proteome sequences by a method comprising: (a) aligning the population of viral proteome sequences; (b) creating, for each sequence in the alignment, a set of 9-amino acid subsequences (“9-mers”) starting with the N-terminal amino acid, each subsequence overlapping the preceding subsequence by eight amino acids such that each sequence of length l in the alignment contains (1-8) 9-mers; (c) calculating a frequency for each unique 9-mer starting at a position i in each sequence of the alignment and identifying the two or more most common unique 9-mers at each position; (c) (l) wherein frequency is calculated as the number of times the unique 9-mer occurs at position i in the alignment divided by the total number of sequences in the alignment; (d) calculating a multivalent conservation for each position by summing the proportion of sequences in the alignment containing either of the two or more most common unique 9-mers; (e) creating an alignment of conserved regions by extracting the sequences in the alignment having a multivalent conservation of greater than 80% or greater than 90%; (f) determining a frequency for each pair of unique 9-mers at each position in the alignment of conserved regions; (g) connecting 9-mer pairs in adjacent positions of the alignment of conserved regions that share an overlap of eight amino acids; (h) creating a directed acyclic graph in which each 9-mer pair is a node and the edges between adjacent nodes are formed from the connected 9-mer pairs in the adjacent positions with the weight of each edge equal to the frequency of the downstream 9-mer pair, (h) (1) adding a source node and connecting it with all of the nodes in the first position, (h) (2) adding a sink node and connecting it with all of the nodes in the last position, and (h) (3) negating all of the weights; (i) finding an optimal path in the directed acyclic graph from the source node to the sink node where the optimal path is defined in terms of the sum of the frequencies of all 9-mer pairs in the directed acyclic graph; (j) building a multivalent antigen by connecting two or more 9-mers in adjacent positions within the optimal multivalent 9-mer path if they share an overlap of eight amino acids, thereby creating two or more sequences of connected 9-mers which together form the multivalent antigen; and (k) optionally, rearranging the polypeptide segments to reduce or avoid the creation of deleterious epitopes at junctions between polypeptide segments. In some embodiments, the multivalent conservation is bivalent conservation and wherein the multivalent antigen is a bivalent antigen. In some embodiments, in step (a) the conserved regions are further defined by performing one or more of the following steps: (i) removing segments of fewer than 35 amino acids in length, e.g., from 9 amino acids to 10, 15, 20, 25, 30 or 35 amino acids in length; (ii) removing segments determined to have less than 90% multivalent (e.g., bivalent) conservation; (iii) removing segments determined to be weakly immunogenic or non-immunogenic, e.g., as demonstrated in in vitro or in vivo; and/or (iv) including additional segments determined to be immunogenic, e.g., as demonstrated in in vitro or in vivo. In some embodiments, the step of rearranging the peptide segments to reduce or avoid creation of deleterious epitopes is performed by a method comprising one or more of in silico HLA binding analysis and human proteome cross-recognition analysis. In some embodiments, the fusion polypeptide design method further comprises the step of inserting a linker sequence between one or more adjacent segments. In some embodiments, the fusion polypeptide design method further comprises improving the multivalent (e.g., bivalent) antigen produced in step (h) by removing junctional 9-mers that bind to a specific HLA allele with a predicted IC50 value of less than about 1000 nM or having a percentile rank within the top 5% in a population of polypeptide segments. In some embodiments, the method further comprises improving the multivalent (e.g., bivalent) antigen produced in step (h) by removing 9-mers that have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity with human peptides or that have the same T cell receptor (TCR) facing residues with human proteins. In some embodiments, the fusion polypeptide design method further comprises the step of rearranging the polypeptide segments to reduce or avoid the creation of deleterious epitopes at junctions between polypeptide segments. In some embodiments, the step of rearranging the peptide segments to reduce or avoid creation of deleterious epitopes is performed by a method comprising one or more of in silico HLA binding analysis and human proteome cross-recognition analysis. In some embodiments, the fusion polypeptide design method further comprises the step of identifying variant subsequences within one or more regions of sequence conservation in an intrapatient population of polypeptide sequences encoded by a viral gene, e.g., using deep sequencing data. In some embodiments, the fusion polypeptide design method further comprises the step of identifying conserved regions of a polypeptide encoded by a viral gene, such that at least 70% of the variant subsequences within the one or more regions of sequence conservation in the intrapatient population are within the bivalent polypeptide segments. In some embodiments, the fusion polypeptide design method further comprises the step of shortening the length of the fusion polypeptide, e.g., by at least 10%, 15%, 20%, 25%, 30%, or more, retaining polypeptide segment subsequences comprising epitopes (i) predicted in silico, and (ii) confirmed in vitro. With respect to the fusion polypeptide design methods, in some embodiments, the one or more viral target antigens are from a mammalian virus, e.g., a human virus. In some embodiments, the one or more viral target antigens are from a virus selected from human immunodeficiency virus (HIV), hepatitis B virus (HBV), human papillomavirus (HPV), herpes simplex virus (HSV), Ebola virus, Zika virus and Chikungunya virus. In some embodiments, the interpatient virus population is from a population of patients who have not received antiretroviral therapy (ART). In some embodiments, the interpatient virus population is from a population of patients who have received antiretroviral therapy (ART). Further provided are fusion polypeptides made according to the fusion polypeptide design methods, described herein, wherein the fusion polypeptide elicits an immune response against a virus in a mammal, e.g., a human.

### BRIEF DESCRIPTION OF THE DRAWINGS

FIG. 1 illustrates a 6-step workflow for designing a fusion polypeptide to elicit an antiviral response.

FIG. 2 illustrates a representative methodology of a population-based vaccine construct approach.

FIG. 3 illustrates the steps of the conserved walking analysis (CWA) algorithm, as described herein.

FIGS. 4A-4B. FIG. 4A illustrates how “bivalent conservation” can be determined based on the prevalence of the two most common 9-mers among all considered viral sequences in a population. FIG. 4A discloses the sequences corresponding to Patients 1-10 as SEQ ID NOS 168-169, 169-170, 169, 169, 171-172 and 172-173, respectively, in order of appearance. FIG. 4A also discloses “QNLQGQMVH” as SEQ ID NO: 174, “QNIQGQMVH” as SEQ ID NO: 175 and “PNIQGQMVH” as SEQ ID NO: 176. FIG. 4B illustrates how the conserved regions are identified based on the “bivalent conservation” distribution across 9-mer positions. HIV-1 Gag p24 was used as the representative protein.

FIGS. 5A-5C. FIG. 5A illustrates unique 9-mers extracted from aligned natural sequences. FIG. 5A discloses SEQ ID NOs 177-183, respectively, in order of columns. FIG. 5B illustrates a directed acyclic graph built based on 9-mer pair nodes and their connection. FIG. 5B discloses “GIIIIIIII” as SEQ ID NO: 180, “AIIIIIIII” as SEQ ID NO: 181, “IIIIIIIIK” as SEQ ID NO: 182, “IIIIIIIIH” as SEQ ID NO: 183 and “IIIIIIIIR” as SEQ ID NO: 184. FIG. 5C illustrates how 9-mers in connected 9-mer pairs are connected. When there are two options available for the connection, the ultimate connection is determined by the prevalence of each connection in naturally occurring sequences. FIG. 5C discloses “GIIIIIIII” as SEQ ID NO: 180, “AIIIIIIII” as SEQ ID NO: 181, “IIIIIIIIK” as SEQ ID NO: 182 and “IIIIIIIIH” as SEQ ID NO: 183.

FIG. 6 illustrates the results of human proteome cross-recognition analysis. FIG. 6 discloses SEQ ID NOs 185-188, respectively, in order of columns.

FIG. 7 illustrates how polypeptide segment arrangement analysis can reduce or eliminate possible presentation of deleterious or undesirable epitopes in junction regions.

FIG. 8 provides a schematic of viral vectors containing fusion proteins of immunogen version 1 (e.g., SEQ ID NOs: 94-101). Fusion polypeptides of immunogen version 1 have twelve conserved regions within HIV-1 Gag, Pol and Nef in the range of 516 to 527 amino acids in length that have been rearranged to reduce or minimize overall junctional responses. A total of four vectors to enable a bi-valent, heterologous prime/boost vaccine regimen.

FIG. 9 provides a flow diagram illustrating the basic methodology of the approach for designing the fusion proteins of immunogen version 2 (e.g., SEQ ID NOs: 82-89). A set of 9-mers is selected from conserved regions and combined to form the fusion polypeptides of immunogen version 2, a subset of immunogen version 1. The sequences and gene regions included in immunogen version 2 are provided in Table E.

FIGS. 10A-10D illustrate the regions of HIV encoded proteins Pol (A-B), Gag (C) and Nef (D) that were selected for the immunogen version 2 by combining deep sequencing data and immunogenicity data. Solid horizontal black line=intra-patient conservation (evaluated by coverage of intra-patient 9-mer variants with bi-valent vaccine). Grey horizonal line=inter-patient conservation. Solid vertical black bars=LANL responses. White vertical bars=ELISpot responses. Horizonal bar (diagonal stripes)=regions maintained in immunogen version 2.

FIG. 11 provides a schematic of viral vectors containing fusion proteins of immunogen version 2 (e.g., SEQ ID NOs: 82-89). Fusion polypeptides of immunogen version 2 have eight conserved regions within HIV-1 Gag, Pol and Nef in the range of 351 to 365 amino acids in length that have been rearranged to reduce or minimize overall junctional responses. A total of four vectors to enable a bi-valent, heterologous prime/boost vaccine regimen.

FIGS. 12A-12B illustrate that median responses remain stable with size reduction of the fusion polypeptides of immunogen version 1 to the fusion polypeptides of immunogen version 2. 12A. The ability of the fusion polypeptides of immunogen version 1 to induce T-cell responses was tested in vitro for a total of 93 donor PBMCs. The dash line shows the median number of responses recognized. Immune responses are detected in 73/93 (78.49%) participants for immunogen version 1. 12B. The fusion polypeptides of immunogen 2 were assessed in silico for the ability to induce T-cell responses in the same participants. The dash line shows the median number of responses recognized. Immune responses are detected in 71/93 (76.34%) participants for immunogen version 2.

FIG. 13 provides a flow diagram illustrating the basic methodology of the approach for designing the fusion proteins of immunogen version 3 (e.g., SEQ ID NOs: 90-93). A set of 9-mers is selected from conserved regions and combined to form the fusion polypeptides of immunogen version 3, a subset of immunogen version 1. The sequences and gene regions included in immunogen version 3 are provided in Table E.

FIGS. 14A-14D illustrate the regions of HIV encoded proteins Pol (A-B), Gag (C) and Nef (D) that were selected for the immunogen version 3 by combining deep sequencing data and immunogenicity data. Solid horizontal black line=intra-patient conservation (evaluated by coverage of intra-patient 9-mer variants with bi-valent vaccine). Grey horizonal line=inter-patient conservation. Solid vertical black bars=LANL responses. Grey vertical bars=ELISpot responses. Horizonal open bar=regions maintain in improved immunogen.

FIG. 15 provides a schematic of viral vectors containing the fusion proteins of immunogen version 3 (e.g., SEQ ID NOs: 90-93). Schematic representation of immunogen 3 version constructs. The fusion polypeptides of immunogen version 3 have four conserved regions within HIV-1 Gag, Pol and Nef in the range of 391 to 394 amino acids in length that have been rearranged to minimize overall junctional responses. A total of two vectors to enable a bi-valent, heterologous prime/boost vaccine regimen.

FIG. 16 illustrates polypeptide segments encoded by the HIV-1 Gag gene used in the fusion polypeptide constructs described herein. The Gag HIV-1 HXB2 reference polypeptide (SEQ ID NO: 1) sequence is underlined. Amino acid residues corresponding to Gag HIV-1 HXB2 reference polypeptide residues 54-146 and 370-500, and subsequences and fragments thereof, are not included in the herein described polypeptide segments. FIG. 16 discloses SEQ ID NOs 1 and 4-7, respectively, in order of appearance.

FIGS. 17A-17C illustrate polypeptide segments encoded by the HIV-1 Pol gene used in the fusion polypeptide constructs described herein. The Pol HIV-1 HXB2 reference polypeptide (SEQ ID NO: 2) sequence is underlined. Amino acid residues corresponding to Pol HIV-1 HXB2 reference polypeptide residues 1-55, 118-128, 321-366, 432-541, 607-682, 709-746, 828-839 and 921-931, and subsequences and fragments thereof, are not included in the herein described polypeptide segments. FIG. 17A-17C discloses SEQ ID NOs 2, 8-21, 24-25, 189, 23 and 26-27, respectively, in order of appearance.

FIG. 18 illustrates polypeptide segments encoded by the HIV-1 Nef gene used in the fusion polypeptide constructs described herein. The Nef HIV-1 HXB2 reference polypeptide, having a tryptophan (W) at position 124 (SEQ ID NO: 3) sequence is underlined. Amino acid residues corresponding to Nef HIV-1 HXB2 reference polypeptide residues 1-63, 100-116 and 149-206, and subsequences and fragments thereof, are not included in the herein described polypeptide segments. FIG. 18 discloses SEQ ID NOs 3, 30-31, 28-29 and 32-33, respectively, in order of appearance.

FIG. 19 illustrates a schematic representation of compound fusion polypeptides containing HIV-1 immunogen version 1 polypeptide sequences (e.g., SEQ ID NOs: 105, 107, 109, 111 and 206-209). The depicted compound fusion polypeptides have 12 conserved regions within HIV-1 Gag, Pol and Nef that have been rearranged to minimize overall junctional responses. All four vectors were used for transduction of monocyte-derived dendritic cells (moDCs) in CD8+T cell priming assays and are collectively labeled as “post vaccination” in assays corresponding to these polypeptide sequences. FIG. 19 discloses “AAA” as SEQ ID NO: 48 and “AAY” as SEQ ID NO: 49.

FIGS. 20A-20B. Ad5 vectors expressing compound fusion polypeptides of SEQ ID NO: 105, 107, 109 or 111 were used to transduce moDCs to evaluate expression of the transgene and transduction efficiency. Expression efficiency was assessed by Gag p24 ELISA (N=2) (FIG. 20A). The y-axis represents Gag p24 concentration (pg/ml) detected in moDC lysates at day 3 post transduction with Ad5 vectors expressing compound fusion polypeptides of SEQ ID NO: 105, 107, 109 or 111 (▪) or empty vector control (A) at multiplicity of infection (MOI) of 500. FIG. 20B illustrates representative moDC transduction efficiency using GFP expressing Ad5 viral vectors at MOI of 500 in N=36 human donors at day 3 post-transduction. Proportion of cells expressing GFP by flow cytometry is shown on the y-axis. The x-axis represents vaccine immunogen constructs of conserved regions of SEQ ID NOs: 105, 107, 109 and 111 (=) or empty vector control (A) at multiplicity of infection (MOI) of 500, or untransduced (.). Transduction efficiency was determined by evaluation of percent GFP expression of transduced moDCs by flow cytometry (N=36). The amino acid sequences are provided in Table F.

FIG. 21 illustrates a schematic representation of fusion polypeptides containing HIV-1 immunogen version 2 fusion polypeptides (SEQ ID NOs: 82-89). The depicted compound fusion polypeptides have 12 conserved regions within HIV-1 Gag, Pol and Nef that have been rearranged to minimize overall junctional responses. All eight vectors were used for transduction of moDCs in CD8+T cell priming assays and are collectively labeled as “post vaccination” in assays using these sequences. FIG. 21 discloses “AAA” as SEQ ID NO: 48, “QEE” as SEQ ID NO: 51, “KILQEE” as SEQ ID NO: 198 and “AAQEE” as SEQ ID NO: 199.

FIGS. 22A-22B. Ad5 vectors expressing compound fusion polypeptides of SEQ ID NO: 82-89 were used to transduce moDCs to evaluate expression of the transgene and transduction efficiency. Expression efficiency was assessed by Gag p24 ELISA (N=2) (FIG. 22A). The y-axis represents Gag p24 concentration (pg/ml) detected in moDC lysates at day 3 post transduction with Ad5 vectors expressing compound fusion polypeptides of SEQ ID NOs: 82-89 (=) or empty vector control ( ) at multiplicity of infection (MOI) of 500. FIG. 22B illustrates representative moDC transduction efficiency using GFP expressing Ad5 viral vectors at MOI of 500 in N=3 human donors at day 3 post-transduction. Proportion of cells expressing GFP by flow cytometry is shown on the y-axis. The x-axis represents vaccine immunogen constructs of conserved regions of SEQ ID NOs: 82-89 (=) or empty vector control (4) at multiplicity of infection (MOI) of 500, or untransduced (.). The amino acid sequences are provided in Table E.

FIG. 23 illustrates a schematic representation of fusion polypeptides containing HIV-1 immunogen version 3 fusion polypeptides (SEQ ID NOs: 90-93). All four vectors were used for transduction of moDCs in CD8+T cell priming assays and are collectively labeled as “post vaccination” in assays using these sequences. FIG. 23 discloses “QEE” as SEQ ID NO: 51, “AAQEE” as SEQ ID NO: 199 and “AAY” as SEQ ID NO: 49.

FIGS. 24A-24B. Ad5 vectors expressing compound fusion polypeptides of SEQ ID NO: 90-93 were used to transduce moDCs to evaluate expression of the transgene and transduction efficiency. Expression efficiency was assessed by Gag p24 ELISA (N=2) (FIG. 24A). The y-axis represents Gag p24 concentration (pg/ml) detected in moDC lysates at day 3 post transduction with Ad5 vectors expressing compound fusion polypeptides of SEQ ID NOs: 82-89 (=) or empty vector control (A) at multiplicity of infection (MOI) of 500. FIG. 24B illustrates representative moDC transduction efficiency using GFP expressing Ad5 viral vectors at MOI of 500 in N=3 human donors at day 3 post-transduction. Proportion of cells expressing GFP by flow cytometry is shown on the y-axis. The x-axis represents vaccine immunogen constructs of conserved regions of SEQ ID NOs: 90-93 (=) or empty vector control (4) at multiplicity of infection (MOI) of 500, or untransduced (·). The amino acid sequences are provided in Table E.

FIG. 25 illustrates a schematic representation of fusion polypeptides containing HIV-1 immunogen version 1 fusion polypeptide sequences. The fusion polypeptides of SEQ ID NOs: 94 and 95 represent bivalent sequences within HIV-1 Gag, Pol and Nef; SEQ ID NOs: 96 and 97 represent bivalent sequences within HIV-1 Pol and Nef. The fusion polypeptides of SEQ ID NOs: 94-97 can be combined to form a priming sequence. The fusion polypeptides of SEQ ID NOs: 98 and 99 represent bivalent sequences within HIV-1 Gag, Pol and Nef. The fusion polypeptides of SEQ ID NOs: 100 and 101 represent bivalent sequences within HIV-1 Pol and Nef. The fusion polypeptides of SEQ ID NOs: 98-101 can be combined to form a boosting sequence that can be used following priming with the fusion polypeptides of SEQ ID NOs: 94-97. Bivalent sequences are designed to cover >80% of inter patient diversity in viral sequences, and gene segments used to generate the polypeptide fusion constructs are rearranged in order to minimize creation of de novo epitopes that react with the human protein and to minimize boosting of junctional responses in prime boost sequences. FIG. 25 discloses “AAA” as SEQ ID NO: 48 and “AAY” as SEQ ID NO: 49.

FIG. 26 illustrates immunization schedule in Balb/c mice. Mice were immunized intramuscularly (IM) in right and left quadriceps at 1×10⁹ PFU with Ad5 vectors expressing HIV-1 fusion polypeptide sequences as indicated. Splenocytes were collected at day 16 after immunization and responses against HIV Gag, Pol and Nef antigens were measured by IFN-γ ELISpot Assay.

FIGS. 27A-B illustrate results of immunogenicity testing in Balb/c mice by IFN-γ ELISpot with peptide stimulation with either HIV-1 Gag (i and ii), Nef (vii and viii) (FIG. 27A), Pol (protease/RT) (iii and iv), or Pol (integrase) (v and vi) (FIG. 27B) peptides. The fusion polypeptides of SEQ ID NOs: 94-95 (Seq 94, Seq 95) were used as bivalent priming sequences. The fusion polypeptides of SEQ ID NOs: 98-99 (Seq 98, Seq 99) were used as boosting sequences. The Y-axis represents magnitude of IFN-γ responses against the specific peptide pool stimulus as number of spot forming colonies (SFC) per 106 splenocytes. Peptide specific values were obtained by subtracting no peptide stimulated control to exclude nonspecific responses. The X-axis indicates the individual vaccine constructs used for in vivo priming against which peptide specific responses were studied. All vectors were immunogenic inducing robust responses to HIV-1 Gag responses, with weaker but detectable responses to Pol proteins (Protease, RT and integrase). No responses were detected to HIV-Nef in this model likely due to previously described immunodominance patterns for HIV-1 Gag epitopes in Balb/c mice and reflecting the lack of Nef epitopes that can be presented in Balb/c mice.

FIG. 28 illustrates results of immunogenicity testing in Balb/c mice by IFN-γ ELISpot with peptide stimulation with either HIV-1 Pol (protease/RT) (i and ii), Pol (integrase) (iii and iv) or Nef (v and vi) peptides. The fusion polypeptides of SEQ ID NOs: 96-97 (Seq 96, Seq 97) were used as bivalent priming sequences. The fusion polypeptides of SEQ ID NOs: 100-101 (Seq 100, Seq 101) were used as boosting sequences. The Y axis represents magnitude of IFN-γ responses against the specific peptide pool stimulus as number of spot forming colonies (SFC) per 106 Splenocytes. Peptide specific values were obtained by subtracting no peptide stimulated control to exclude nonspecific responses. The X-axis indicates the individual vaccine constructs used for in vivo priming against which peptide specific responses were studied. All vectors were immunogenic inducing robust responses to HIV-1 Pol, particularly protease and RT peptides with weaker responses detected to integrase peptides. No responses were detected to HIV-Nef in this model likely due to previously described immunodominance patterns for HIV-1 Gag epitopes in Balb/c mice and reflecting the lack of Nef epitopes that can be presented in Balb/c mice.

FIG. 29 illustrates a representative immunization schedule in Balb/c mice. Mice were immunized I.M. in right and left quadriceps at 1×10⁹ PFU with Ad5 vectors expressing HIV-1 sequences as indicated. Individual vectors (i) were tested for immunogenicity as a single dose to prime the response with spleens harvested on Day 16 for analysis. Combination vectors (ii) were tested in a homologous vector prime-boost schedule, with boosting on Day 29 and spleens harvested for analysis on Day 36. Responses against Gag, Pol and Nef antigens were measured by IFN-γ ELISpot Assay and ICS/Flow cytometry.

FIGS. 30A-30B illustrate immunogenicity assessed following immunization either by single Ad5 vector prime alone (A) or in a homologous Ad5 vector prime-boost combination (B). Seq 94-F2A-95 (tPA-SEQ ID NO: 105) and Seq 98-F2A-99 (tPA-SEQ ID NO: 109) were used as a prime-boost pair. Vectors were tested for immunogenicity in Balb/c mice by IFN-γ ELISpot with peptide stimulation with either HIV-1 Gag, (i) and (ii); Pol (protease/RT, (iii) and (iv); Pol (integrase, (v) and (vi); or Nef (vii and viii) peptides. The Y axis represents magnitude of IFN-γ responses against the specific peptide pool stimulus as number of spot forming colonies (SFC) per 106 Splenocytes. Peptide specific values were obtained by subtracting no peptide stimulated control to exclude nonspecific responses. The X-axis indicates the individual vaccine constructs used for in vivo priming against which peptide specific responses were studied. All vectors were immunogenic inducing robust responses to HIV-1 Gag responses, with weaker but detectable responses to Pol proteins (protease, RT and integrase). No responses were detected to HIV-Nef in this model likely due to previously described immunodominance patterns for HIV-1 Gag epitopes in Balb/c mice and reflecting the lack of Nef epitopes that can be presented in Balb/c mice. Responses were enhanced by homologous boosting with Ad5 vectors expressing the boost sequence.

FIGS. 31A-31B illustrate immunogenicity is assessed following immunization either by single Ad5 vector prime alone or in a homologous Ad5 vector prime-boost combination. Seq 96-F2A-97 (tPA-SEQ ID NO: 107) and Seq 100-F2A-101 (tPA-SEQ ID NO: 111) were used as a prime boost pair. Vectors were tested for immunogenicity in Balb/c mice by IFN-γ ELISpot with peptide stimulation with either HIV-1 Pol (protease/RT, (i) and (ii); Pol (integrase, (iii) and (iv); or Nef (v and vi) peptides. The Y-axis represents magnitude of IFN-γ responses against the specific peptide pool stimulus as number of spot forming colonies (SFC) per 106 Splenocytes. Peptide specific values were obtained by subtracting no peptide stimulated control to exclude nonspecific responses. The X-axis indicates the individual vaccine constructs used for in vivo priming against which peptide specific responses were studied. All vectors were immunogenic inducing robust responses to HIV-1 Pol, particularly Protease and RT peptides, with weaker responses detected to integrase peptides. The responses observed to the Protease and RT were weaker than previously observed by Seq 96 and Seq 97 when not combined. The immunogenicity responses were boosted in sequential dosing with Seq 96-F2A-97 and Seq 100-F2A-101, however. No responses were detected to HIV-Nef in this model likely due to previously described immunodominance patterns for HIV-1 Gag epitopes in Balb/c mice and reflecting the lack of Nef epitopes that can be presented in Balb/c mice.

FIGS. 32A-32C illustrate HIV-1 Gag immunogenicity by intracellular cytokine staining (ICS) following vaccination with single vectors, Seq 94-F2A-95 (tPA-SEQ ID NO:105) or Seq 98-F2A-99 (tPA-SEQ ID NO: 109), as well as homologous prime-boost. The Y axis represents proportion of CD8+ (FIG. 32A i, ii and FIG. 32C iii) or CD4+ (FIG. 32B iv, v and FIG. 32C vi) T cells exhibiting HIV-Gag specific responses by expressing cytokines IFN-γ, IL-2 and TNF-α, either as individual cytokines or combination of cytokines, as shown on X axis. HIV-Gag specific values were obtained by subtracting no peptide stimulated control to exclude nonspecific responses. Strong CD4+ and CD8+ T cell responses were generated in response to vaccination, with demonstrated polyfunctionality most robust in CD8+T cells. Homologous prime-boost enhanced polyfunctionality of the CD8+ T cell responses.

FIGS. 33A-33C illustrate HIV-1 Pol (protease and RT) immunogenicity by ICS following vaccination with single vectors, Seq 94-F2A-95 (tPA-SEQ ID NO: 105) or Seq 98-F2A-99 (tPA-SEQ ID NO: 109), as well as homologous prime-boost. The Y axis represents proportion of CD8+ (FIG. 33A i, ii and FIG. 33C iii) or CD4+ (FIG. 33B iv, v and FIG. 33C vi) T cells exhibiting HIV-Pol (protease and RT) specific responses by expressing cytokines IFN-γ, IL-2 and TNF-α, either as individual cytokines or combination of cytokines, as shown on X-axis. HIV-Pol (Protease and RT) specific values were obtained by subtracting no peptide stimulated control to exclude nonspecific responses. CD4+ and CD8+ T cell responses were generated in response to vaccination. Responses are weaker than Gag responses and tend to be monofunctional, with limited boosting.

FIGS. 34A-34C illustrate HIV-1 Pol (integrase) immunogenicity by ICS following vaccination with single vectors, Seq 94-F2A-95 (tPA-SEQ ID NO: 105) or Seq 98-F2A-99 (tPA-SEQ ID NO: 109), as well as homologous prime-boost. The Y-axis represents proportion of CD8+ (FIG. 34A i, ii and FIG. 34C iii) or CD4+ (FIG. 34B iv, v and FIG. 34C vi) T cells exhibiting HIV-Pol (integrase) specific responses by expressing cytokines IFN-γ, IL-2 and TNF-α, either as individual cytokines or combination of cytokines, as shown on the X-axis. HIV-Pol (integrase) specific values were obtained by subtracting no peptide stimulated control to exclude nonspecific responses. CD4+ and CD8+ T cell responses were generated in response to vaccination. Responses are weaker than Gag responses and tend to be monofunctional, with limited boosting.

FIGS. 35A-35C illustrate HIV-1 Nef immunogenicity by ICS following vaccination with single vectors, Seq 94-F2A-95 (tPA-SEQ ID NO:105) or Seq 98-F2A-99 (tPA-SEQ ID NO: 109), as well as homologous prime boost. The Y-axis represents proportion of CD8+ (FIG. 35A i, ii and FIG. 35C iii) or CD4+ (FIG. 35B iv, v and FIG. 35C vi) T cells exhibiting HIV-Nef specific responses by expressing cytokines IFN-γ, IL-2 and TNF-α, either as individual cytokines or combination of cytokines, as shown on the X-axis. HIV-1 Nef specific values were obtained by subtracting no peptide stimulated control to exclude nonspecific responses. Very low proportion of CD4+ and CD8+ T cell responses were generated in response to vaccination. Responses are weaker than Gag responses and tend to be monofunctional, with limited boosting.

FIGS. 36A-36C illustrate HIV-1 Pol (protease and RT) immunogenicity by ICS following vaccination with single vectors, Seq 96-F2A-97 (tPA-SEQ ID NO: 107) and Seq 100-F2A-101 (tPA-SEQ ID NO: 111), as well as homologous vector prime-boost. The Y-axis represents proportion of CD8+ (FIG. 36A i, ii and FIG. 36C iii) or CD4+ (FIG. 36B iv, v and FIG. 36C vi) T cells exhibiting HIV-Pol (protease and RT) specific responses by expressing cytokines IFN-γ, IL-2 and TNF-α, either as individual cytokines or combination of cytokines, as shown on X-axis. HIV-1 Pol (protease and RT) specific values were obtained by subtracting no peptide stimulated control to exclude nonspecific responses. CD4+ and CD8+ responses were generated in response to vaccination. Seq 100-F2A-101 (tPA-SEQ ID NO: 111) demonstrated strong immunogenicity with polyfunctionality (>2 cytokines produced). Homologous vector prime-boost with the vectors expressing the fusion polypeptides enhanced both CD4+ and CD8+ T cell responses.

FIGS. 37A-37C illustrate HIV-1 Pol (Integrase) immunogenicity by ICS following vaccination with single vectors, Seq 96-F2A-97 (tPA-SEQ ID NO: 107) and Seq 100-F2A-101 (tPA-SEQ ID NO: 111), as well as homologous vector prime boost. The Y-axis represents proportion of CD8+ (FIG. 37A i, ii and FIG. 37C iii) or CD4+ (FIG. 37B iv, v and FIG. 37C vi) T cells exhibiting HIV-Pol (integrase) specific responses by expressing cytokines IFN-γ, IL-2 and TNF-α, either as individual cytokines or combination of cytokines, as shown on X-axis. HIV-1 Pol (integrase) specific values were obtained by subtracting no peptide stimulated control to exclude nonspecific responses. CD4+ and CD8+ responses were generated in response to vaccination. Seq 100-F2A-101 (tPA-SEQ ID NO: 111) demonstrated strong immunogenicity with polyfunctionality (>2 cytokines produced). Homologous vector prime-boost with the vectors expressing the fusion polypeptides enhanced both CD4+ and CD8+ T cell responses.

FIGS. 38A-38C illustrate HIV-1 Nef Immunogenicity by ICS following vaccination with single vectors, Seq 96-F2A-97 (tPA-SEQ ID NO: 107) and Seq 100-F2A-101 (tPA-SEQ ID NO: 111), as well as homologous vector prime boost. The Y axis represents proportion of CD8+FIG. 38A i, ii and FIG. 38C iii) or CD4+ (FIG. 38B iv, v and FIG. 38C vi) T cells exhibiting HIV-Nef specific responses by expressing cytokines IFN-γ, IL-2 and TNF-α, either as individual cytokines or combination of cytokines, as shown on X-axis. HIV-1 Nef specific values were obtained by subtracting no peptide stimulated control to exclude nonspecific responses. Low levels of CD4+ and CD8+ T cell responses were generated in response to vaccination and more responses were seen with CD4 T cells. Seq 100-F2A-101 (tPA-SEQ ID NO: 111) demonstrated and induced mostly monokine production. Homologous vector prime-boost with the vectors expressing the fusion polypeptides enhanced both CD4+ and CD8+ T cell responses.

FIG. 39 provides a schematic of a protocol established for moDC-T cell priming assays for testing immunogenicity of all conserved regions vaccine constructs followed by individual epitope mapping using 384-well ELISpot assays.

FIG. 40 provides a summary of breadth of responses in ex vivo analysis of PBMCs from N=93 participants following in vitro priming with moDCs. Heat map representing the breadth of T cell responses to Gag, Pol and Nef conserved antigens following in vitro priming with moDCs transduced with Ad5-empty vector MOI 500 (pre-vaccine) or Ad5 viral vectors expressing SEQ ID NOs: 105, 107, 109 and 111, each at MOI=500 (post-vaccine). Each matched row in the heat map represents responses from a single participant and breadth of responses are categorized as 0, 1-3. 4-6, 7-9, 10-12 or 13-16. Positive ELISpot responses were defined as >3-fold background levels as described in detail in Example 9.

FIG. 41 illustrates characterization of the breadth of immune responses targeted to HIV-1 Gag, Pol and Nef antigens post priming with moDCs transduced with Ad5 viral vectors expressing empty vectors (pre-vaccine) or conserved regions vaccines (SEQ ID NOs: 105, 107, 109 and 111, each at MOI=500 (post vaccine) in N=93 participants. Also enumerated is the fraction of participants that exhibited responses to ≥3 or 0 epitopes in each antigen following in vitro priming with vaccine immunogen sequences. Positive ELISpot responses were defined as >3-fold background levels. Each point represents one donor. Wilcoxon matched pairs signed rank test **** p<0.0001.

FIG. 42 illustrates breadth of responses to Gag (.), Pol (A) and Nef (7) defined as number of de novo recognized peptides (excluding pre-existing baseline responses) assessed by IFN-γ ELISpot assay on day 10 following co-culture of PBMCs with Ad5 vaccine vector transduced autologous moDCs expressing conserved regions constructs (SEQ ID NOs: 105, 107, 109 and 111, each at MOI=500). Each point represents one donor. Median with interquartile range are shown.

FIGS. 43A-43B illustrate results of an in vitro HIV-1 viral inhibition assay staining for Gag-p24 within target cells. Autologous CD4+ T cells were infected in vitro with HIV-1 BaL, cultured alone or in the presence of vaccine or empty vector primed CFSE labeled CD8+T cells for 3 days and analyzed for infection using flow cytometry. Representative flow cytometry plots illustrating the gating strategy to define Gag p24+infected CD4+T cells (HIV-1 Gag+ cells with down-regulated surface CD4 expression) within target cells (CFSE-CD8-cells).

FIGS. 44A-44B illustrate fraction of residual HIV-1 Gag+CD4-T cells after 3 days of co-culture with enriched CD8+T cells obtained after priming with vaccine (SEQ ID NOs: 105, 107, 109 and 111, or empty vector control each at MOI-500 for N=51 participants normalized to infected CD4+T cells cultured without CD8+T cells. Each condition was completed in technical duplicates or triplicates depending on cell availability. Also illustrated correlation between % residual Gag+ cells (FIG. 44A) and breadth of total, Gag and Pol responses (FIG. 44B). Correlations evaluated by 2-sided Spearman rank-correlation tests without Bonferroni adjustments.

FIGS. 45A-45B illustrates breadth of responses to Gag, Pol and Nef assessed by IFN-γ ELISpot assay on day 10 following co-culture of PBMCs with Ad5 vaccine vector transduced autologous moDCs expressing conserved regions constructs, SEQ ID NO: 82-89 (FIG. 45A) and SEQ ID NO: 90-93 (FIG. 45B), each at MOI=500). Each point represents one donor. Median with interquartile range are shown.

FIG. 46 provides a schematic of immunization schedule prime-boost with arenavirus vectors in non-human primates (NHPs). Indian-origin healthy rhesus macaques were immunized via intramuscular (I.M.) route with the arenavirus vectors. The Gag and Env expressing vectors (TT2 (replication attenuated Pichinde) and TT1 (replication attenuated LCMV)) were administered on the left quadricep whereas the Pol expressing vectors (TT2 and TT1) were administered on the right quadricep. The doses administered are as below: 1×10⁶ replication competent virus particles (RCV) of TT2 Gag, Env and Pol1/Pol2 vectors, 4×10⁶ RCV of TT1 Gag, Env, and 2×10⁶ RCV of TT1 Pol1/Pol2. In the placebo group, NHPs were administered placebo buffer solution composed of 10 mM HEPES, 150 mM NaCl, 20 mM Glycine and 0.1% macaque serum albumin. Responses against Gag, Env and Pol antigens were measured by IFN-γ ELISpot Assay.

FIGS. 47A-47B. 47A. Time course of total responses to Gag, Env and Pol was assessed by IFN-γ ELISpot on PBMCs isolated from NHP peripheral blood at two four-week intervals post Ist vaccine dose up to week 32. Median with interquartile range are shown. 47B. The NHP responses in FIG. 47A were classified as moderate or high responders depending on whether the magnitude of peak IFN-γ-ELISpot responses was below or above 1000 SFU/106 PBMCs, respectively. Each line represents one animal that received the arenavirus vaccine vector. Peak response after each immunization remains constant with subsequent boosting.

FIGS. 48A-48D illustrate magnitude of total responses (A) to Gag (B), Env (C) and Pol (D) assessed by IFN-γ ELISpot on PBMCs isolated from NHP peripheral blood at two weeks post vaccine doses 1, 2, 3 and 4. Threshold for positive response is indicated by dotted line and set to observing at least 50 SFU/106 PBMCs for Gag, Env and Pol and to 150 SFU/106 PBMCs for the total SIV-specific response. Each symbol represents one NHP. Median with interquartile range are shown. After four doses of the heterologous vaccine, a positive response to Gag, Env and Pol is observed in at least 21 of 24 NHPs (response rate greater or equal to 87.5%). The magnitude of the Gag and Pol responses observed after doses 2 (TT1) and 3 (TT2) remained stable with each subsequent vaccine boost.

FIGS. 49A-49D illustrate the breadth of total SIV (A), Gag (B), Env (C) and Pol (D)-specific IFN-γ responses at 2 weeks post vaccine boost dose 2 (triangles), dose 3 (squares) and dose 4 (diamond). Statistical analysis by Wilcoxon matched-pairs signed rank test was performed. Data are represented as median±interquartile range (IQR).

FIGS. 50A-50C illustrate (A) Time course of plasma SIV viral load over weeks 0-40 post challenge; (B) Peak viral load at week 2 post challenge; (C) Setpoint viral load measured over weeks 10-40 post challenge. Placebo group is shown as closed triangles and TT2/TT1 vaccine group in closed circles. Data are represented as median+SEM. * p<0.05 and ** p<0.01 in B and C by Mann-Whitney t-test. Median viral load (VL) is indicated above each group in figures B and C.

FIG. 51 illustrates an immunoblot analysis of replication-attenuated arenavirus vectors. Transgene expression of Gag/Nef/Pol fusion polypeptides from stock material (pl) of replication-attenuated-LCMV and replication-attenuated-PICV vectors, was determined by immunoblot staining of the Gag epitope p24 (mAb anti HIV1 p24; Abcam). Whole cell lysates of three biological replicates (1, 2, 3) were analyzed. TT1=replication-attenuated-LCMV; TT2=replication-attenuated-PICV. GNP=Gag/Nef/Pol; PN=Pol/Nef. TT1-HIV-GNP1/PN1 (SEQ ID NOs: 98 and 100); TT1-HIV-GNP2/PN2 (SEQ ID NOs: 99 and 101); TT2-HIV-GNP1/PN1 (SEQ ID NOs: 82 and 86); TT2-HIV-GNP2/PN2 (SEQ ID NOs: 83 and 87).

FIG. 52 illustrates immunoblot analysis of replication-attenuated arenavirus vectors. Transgene expression Gag/Nef/Pol from stock material (pl) of replication attenuated-LCMV and replication attenuated-PICV HIV Immunogen 1 vectors, was determined by immunoblot staining of the Gag epitope p24 (mAb anti HIV1 p24; Abcam). Whole cell lysates of three biological replicates (1, 2, 3) were analyzed. TT1=replication attenuated-LCMV; TT2-replication attenuated-PICV. GNP=Gag/Nef/Pol; PN=Pol/Nef.

FIG. 53 illustrates transgene stability analysis of TT1-HIV-GNP1/PN1 (having transgenes encoding fusion polypeptides of SEQ ID NOs: 98 and 100). Shown are transgene PCR results for 4 biological replicates (1-4) and for indicated passaging levels. The last passage showing ≥50% full length transgene was visually assessed.

FIG. 54 illustrates transgene stability analysis of TT1-HIV-GNP2/PN2 (having transgenes encoding fusion polypeptides of SEQ ID NOs: 99 and 101). Shown are transgene PCR results for 3 biological replicates (1-3) and for indicated passaging levels. The last passage showing ≥50% full length transgene was visually assessed.

FIG. 55 illustrates transgene stability analysis of TT2-HIV-GNP1/PN1 (having transgenes encoding fusion polypeptides SEQ ID NOs: 94 and 95). Shown are transgene PCR results for 3 biological replicates (1-3) and for indicated passaging levels. The last passage showing >50% full length transgene was visually assessed.

FIG. 56 illustrates transgene Stability analysis of TT2-HIV-GNP2/PN2 (having transgenes encoding fusion polypeptides SEQ ID NOs: 95 and 97). Shown are transgene PCR results for 3 biological replicates (1-3) and for indicated passaging levels. Passaging was discontinued at passage level 4. The (presumably) last passage showing ≥50% full length transgene was visually assessed.

FIG. 57 illustrates immunoblot analysis of replication-attenuated arenavirus vectors. Transgene expression Gag/Pol from stock material (P1) and BDS-equivalent passage (P4) of replication-attenuated-LCMV and replication-attenuated-PICV HIV Immunogen 2 vectors, was determined by immunoblot staining of the Gag epitope p24 (mAb anti HIV1 p24; Abcam). Whole cell lysates of biological replicates (1, 2, 3, 4 for TT1-HIV (C2)-GP1/PN1; 1, 2 for TT1-HIV (C2)-GP2/PN2) or representative samples (TT2-HIV (C2)-GP1/PN1 and TT2-HIV (C2)-GP2/PN2) were analyzed. TT1=replication-attenuated-LCMV; TT2=replication-attenuated-PICV. GP=Gag/Pol; PN=Pol/Nef. Ctrls=positive (+) control represents TT1-HIV Immunogen 2 or TT2-HIV Immunogen 2 infected cell lysates; negative (−) control represents uninfected cells.

FIG. 58 illustrates transgene stability analysis of TT1-HIV (C2)-GP1/PN1. A Shown are transgene PCR results for 5 biological replicates (1-5) and for indicated passaging levels. The expected size of the full-length PCR product is 1359 bp (LCMV-GP1 NP-Segment) and 1366 bp (LCMV-PN1 GP-Segment), respectively. The last passage showing ≥50% full length transgene was visually assessed.

FIG. 59 illustrates transgene stability analysis of TT1-HIV (C2)-GNP2/PN2. A Shown are transgene PCR results for 4 biological replicates (1-4) and for indicated passaging levels. The expected size of the full-length PCR product is 1344 bp (LCMV-GP2 NP-Segment) and 1364 bp (LCMV-PN2 GP-Segment), respectively. The last passage showing ≥50% full length transgene was visually assessed.

FIG. 60 illustrates transgene stability analysis of TT2-HIV (C2)-GP1/PN1. A Shown are transgene PCR results for 4 biological replicates (1-3 and 9) and for indicated passaging levels. The expected size of the full-length PCR product is 1456 bp (PICV-GP1 NP-Segment) and 1496 bp (PICV-PN1 GP-Segment), respectively. The last passage showing ≥50% full-length transgene was visually assessed.

FIG. 61 illustrates transgene stability analysis of TT2-HIV (C2)-GP2/PN2. Shown are transgene PCR results for 3 biological replicates (1-3) and for indicated passaging levels. The expected size of the full-length PCR product is 1450 bp (PICV-GP2 NP-Segment) and 1505 bp (PICV-PN2 GP-Segment), respectively. Passaging was discontinued at passage level 6. The (presumably) last passage showing ≥50% full length transgene was visually assessed.

FIG. 62 illustrates a schematic of non-human primate study evaluating combination of TT2/TT1 vaccine replication-attenuated PICV/LCMV arenavirus prime-boost scheme in combination with immune modulators. Each group had 13 rhesus macaques. Vaccine refers to alternating dosing of TT2 (closed circles) and TT1 (open circles). Checkpoint inhibitors aPD1 antibody (triangle) and aCTLA4 antibody (square) were administered immediately after the vaccine. FLT3L-Fc FLT3 agonist (black line) was administered one week before each vaccine dosing.

FIG. 63 illustrates the kinetics of total responses to Gag, Env and Pol was assessed by IFN-γ ELISpot in PBMCs isolated from NHP peripheral blood at two week intervals post 1st vaccine dose up to week 16. Median with interquartile range are shown. Statistical analysis by two-way ANOVA with repeated measures and Dunnett's post-test was performed. * p<0.05, ** p<0.01, *** p<0.001 for comparison of combination group with vaccine alone.

FIGS. 64A-D illustrates the breadth of (A) total SIV, (B) Gag, (C) Env and (D) Pol-specific IFN-γ responses at 2 weeks post last vaccine dose in PBMCs. Vector (Vx) only group in circles, Vx+aPD1 in triangles, Vx+aCTLA4 in squares and Vx+Flt3L in diamonds. Statistical analysis by Kruskal-Wallis test with Dunn's post-test was performed. Data are represented as median+IQR. * p<0.05, ** p<0.01, *** p<0.001, **** p<0.0001.

### DETAILED DESCRIPTION

### 1. Introduction

Herein is described an antiviral vaccine immune design approach that incorporates intra-patient diversity by using deep sequence data to evaluate viral quasispecies and determine the level of viral diversity within an individual. We developed an algorithm that considers both the interpatient as well as intrapatient viral diversity in the design of the vaccine immunogen, as well as immunogenicity of vaccine sequences. Understanding host viral diversity and antigen processing and presentation and T cell priming not only ensures that a vaccine immunogen generates a large number of antigen specific T cells, but also generates antigen specific T cells with cytotoxic activity. In the design of the antiviral vaccine immunogen we have combined computational in silico analysis of viral sequences and binding specificities with in vitro experimental immunology. We established an in vitro vaccine trial model that has enabled us to identify conserved regions that generate the strongest response across a large population. The approach has enabled us to identify novel epitopes within the conserved region that had previously not been recognized. This immunogenicity data has enabled the development of highly conserved, highly immunogenic vaccine immunogens that can be delivered to a broad population.

Provided herein are fusion polypeptides comprising a plurality of polypeptide or peptide segments and related compositions, including immunogenic compositions and pharmaceutical compositions, as well as methods for making the fusion polypeptides and methods for their use to elicit an immunogenic response to a human immunodeficiency virus (HIV-1) in a subject in need thereof. As used herein, an “immunogen” is a substance, such as an antigen, that elicits an immune response or is capable of eliciting an immune response. Also provided are polynucleotides encoding the fusion polypeptides described herein, as well as vectors comprising same.

Provided herein are fusion polypeptides designed to induce an antiviral immune response. The vaccine constructs described herein were designed to provide mathematically-determined improved coverage of predicted T cell epitopes (“PTE”) using the most highly conserved predicted epitopes within a source set of viral proteome sequences. As a paradigm for the methods of designing antiviral immunogens, fusion polypeptides encoded at least two of the HIV-1 genes gag, pol and nef were used. The fusion polypeptides and methods described herein both retain the positional information of the PTE's within the source set of sequences and construct a bivalent set of sequences to improve coverage of conserved PTEs. Accordingly, described herein are multivalent, e.g., bivalent, vaccine constructs that advantageously improve or increase highly conserved PTEs that are most likely to be highly similar to conserved epitopes in the naturally occurring sequences in proteins expressed by viral species amongst a population of patients and within an individual patient, due to both the retained positional information. In addition, the use of only highly conserved PTE sequences amongst HIV-1 species in interpatient populations reduces the likelihood of escape mutants because the highly conserved sequences are more likely to contribute viral structure and function.

Further provided are computational approaches for designing antiviral vaccine immunogens for a highly variable virus, such as HIV-1. The antiviral immunogens can be designed to provide coverage at an individual level, for a group of individuals with a defined set of HLA alleles, or for broad population coverage. In the herein described vaccine immunogen design methods, we define a computational approach for targeting conserved regions within a vaccine sequence using bulk population sequences, e.g., from public databases and internally developed databases. Further, using individual patient deep sequence data we define sequence variability for each potential T cell epitope within the conserved regions. Moreover, we identify regions that may serve as actual epitopes based on likelihood of presentation by the individual host's set of HLA alleles. The likelihood of binding to host HLA defined by publicly available and internally-developed databases, was used to develop deep learning models that model peptide binding per allele. This can be coupled with in-silico, published and/or experimental in-vitro T cell priming data that can define the potential impact of antigen variants in modulating TCR recognition or identify a peptide as an escape variant. This data is used to design a set of peptide immunogens that contain the epitopes and associated epitope variants. The epitope sequences are concatenated or connected in series into a single fusion polypeptide, either directly fused or linked via a linker sequence. Peptide segments are joined in a computationally determined sequential order from N-terminus to C-terminus that reduces or eliminates the creation of junctional epitopes that may mimic human self-antigens and have undesirable effects (e.g., eliciting an autoimmune response or a tolerogenic response).

Unlike similar graph-based approaches to vaccine design, the approaches described herein build segments of connected PTE's using only adjacent PTE's that are also adjacent in the natural sequences. In addition, the present methods first build a bivalent construct consisting of two polypeptides matched to improve or increase coverage at each PTE position in the viral proteome. The bivalent construct itself may be used as a vaccine, as in the constructs described in Examples 1 and 2 below. The bivalent constructs designed by analysis of population-based sequences (e.g., interpatient diversity) identifies population-based conserved sequences that may contribute to viral structure.

The methods described herein can begin with the identification of conserved region bivalent sequences, using a process referred to herein as the “Conservation Analysis” or “Conservation Algorithm.” The methods further can comprise a step of building a bivalent vaccine construct having maximal epitope coverage while retaining the positional information of the PTE's from the natural sequences, using a process referred to referred to herein as a “Conserved Walking Algorithm” or “CWA.”

### 2. Fusion Polypeptides Useful to Promote Immune Response Against Human Immunodeficiency Virus-1 (HIV-1)

Provided herein are fusion polypeptides comprising a plurality of polypeptide or peptide segments encoded by one or more HIV-1 genes. A ‘segment’ of a fusion polypeptide described herein is a contiguous sequence of at least 25 amino acids with respect to a reference sequence, for example HIV-1 HXB2 reference sequences for Gag, Pol and Nef polypeptides, provided herein as SEQ ID NOs: 1-3, respectively. The polypeptides described herein are ‘fusion’ polypeptides in the sense that they are assembled from connected or concatenated polypeptide or peptide segments of two or more HIV-1 proteins, e.g., at least Pol and Nef. With respect to the HIV-1 protein reference sequences, the polypeptide or peptide segments may correspond to discontinuous sequences of the same HIV-1 protein or different HIV-1 proteins. Generally, the fusion polypeptides are non-naturally occurring, and can be synthetic or recombinantly produced.

In various embodiments, immunogenic polypeptides or fusion polypeptides described herein, and/or the polynucleotides encoding such polypeptides, are provided in isolated form. This means that the polypeptide or polynucleotide is at least 50% w/w pure of interfering proteins, cellular and other contaminants arising from its production or purification but does not exclude the possibility that the agent is combined with an excess of pharmaceutical acceptable carrier(s) or other vehicle intended to facilitate its use. The term “isolated,” when applied to a polypeptide or polynucleotide, as described herein, denotes that the polypeptide or polynucleotide is essentially free of cellular components with which it is associated in the natural state. It can be, for example, in a homogeneous state and may be in either a dry or aqueous solution. Purity and homogeneity can be determined using known methods, e.g., analytical chemistry techniques such as polyacrylamide gel electrophoresis, column chromatography, thin layer chromatography, or high-performance liquid chromatography (HPLC) analysis. A protein that is the predominant species present in a preparation is substantially purified. An “isolated” or “purified” polypeptide or polynucleotide is substantially free of other cellular material, or culture medium when produced by recombinant techniques, or chemical precursors or other chemicals when chemically synthesized. In various embodiments, purified polypeptides and/or polynucleotides are at least 60%, 70%, 75%, 80%, 85%, 90%, 91%, 92%, 93%, 94%, 95%, 96%, 97%, 98%, 99%, or 100% (w/w), separated from, purified of, or free of interfering proteins and contaminants from production or purification. Often an immunogenic polypeptides or fusion polypeptides described herein, and/or the polynucleotides encoding such polypeptides, is the predominant macromolecular species remaining after its purification.

a. Polypeptide Segments

With respect to the HIV-1 genes encoding the polypeptide segments used to assemble the herein described fusion polypeptides, in various embodiments, the fusion polypeptides comprise a plurality of polypeptide segments of one or more human immunodeficiency virus-1 (HIV-1) proteins encoded by one or more, e.g. two or more, three or more, HIV-1 genes selected from Gag, Pol and Nef. In some embodiments, the plurality of polypeptide segments is comprised of only polypeptide segments encoded by HIV-1 genes pol and nef, e.g., does not comprise polypeptide segments encoded by HIV-1 genes gag, env, tat, rev, vpr, vif and vpu. In some embodiments, the plurality of polypeptide segments is comprised of only polypeptide segments encoded by HIV-1 genes gag, pol and nef, and does not comprise polypeptide segments encoded by HIV-1 genes env, tat, rev, vpr, vif and vpu.

With respect to the number of polypeptide segments assembled, connected, linked or concatenated into a single fusion polypeptide, in various embodiments, the fusion polypeptides are comprised of at least 4 and up to 6 polypeptide segments, e.g., 4, 5 or 6 polypeptide segments. As appropriate, the polypeptide segments can be arranged in the same order or according to a different order than in the naturally occurring proteins. In various embodiments, the fusion polypeptides comprise from 1 to 4 Pol polypeptide segments, from 1 to 2 Nef polypeptide segments, and optionally, from 1 to 3 Gag polypeptide segments.

With respect to the regions of the polypeptides encoded by an HIV-1 gene selected as polypeptide segments to include in the fusion polypeptides, in various embodiments, the polypeptide segments are derived from conserved regions in a population of viral proteome sequences. In some embodiments, the conserved regions are greater than 80%, e.g., greater than 81%, 82%, 83%, 84%, 85%, 86%, 87%, 88%, 89%, 90%, 91%, 92%, 93%, 94%, 95%, 96%, 97%, 98% or 99% conserved amongst HIV-1 species, e.g., as determined in interpatient populations. As used herein, conserved regions in a polypeptide encoded by an HIV-1 gene refers to the percentage of sequences in a population of sequences containing identical amino acid segments or subsequences e.g., segments 9 amino acids in length or 9-mers as the most prevalent one in a predetermined amino acid segment or subsequence position, where an amino acid segment or subsequence position is determined with respect to a reference sequence, e.g., HIV-1 HXB2 polypeptide sequences, e.g., SEQ ID NOs: 1-3. The start and end positions of the HIV polypeptides identified herein are with respect to HIV-1 HXB2 reference polypeptides, GenBank Accession No. K03455 (ncbi.nlm.nih.gov/nuccore/K03455), provided herein as SEQ ID NOs: 1-3 and identified in Table A. As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer. In various embodiments, the conserved regions are conserved amongst one or more of HIV-1 clades within Group M, e.g., one or more of HIV-1 clades A-K, e.g., one or more of clades A, B, C, D and G, e.g., amongst HIV-1 Group M, clade B, and recombinant forms thereof, e.g., CRF01_AE.

TABLE A
HIV-1 HXB2 reference sequences

| SEQ | | |
|---|---|---|
| ID | | |
| NO: | gene | Amino acid sequence of encoded protein |
| 1 | gag | MGARASVLSGGELDRWEKIRLRPGGKKKYKLKHIVWAS |
| | | RELERFAVNPGLLETSEGCRQILGQLQPSLQTGSEELR |
| | | SLYNTVATLYCVHQRIEIKDTKEALDKIEEEQNKSKKK |
| | | AQQAAADTGHSNQVSQNYPIVQNIQGQMVHQAISPRTL |
| | | NAWVKVVEEKAFSPEVIPMFSALSEGATPQDLNTMLNT |
| | | VGGHQAAMQMLKETINEEAAEWDRVHPVHAGPIAPGQM |
| | | REPRGSDIAGTTSTLQEQIGWMTNNPPIPVGEIYKRWI |
| | | ILGLNKIVRMYSPTSILDIRQGPKEPFRDYVDRFYKTL |
| | | RAEQASQEVKNWMTETLLVQNANPDCKTILKALGPAAT |
| | | LEEMMTACQGVGGPGHKARVLAEAMSQVTNSATIMMQR |
| | | GNERNQRKIVKCFNCGKEGHTARNCRAPRKKGCWKCGK |
| | | EGHQMKDCTERQANFLGKIWPSYKGRPGNFLQSRPEPT |
| | | APPEESFRSGVETTTPPQKQEPIDKELYPLISLRSLFG |
| | | NDPSSQ |
| 2 | pol | FFREDLAFLQGKAREFSSEQTRANSPTRRELQVWGRDN |
| | | NSPSEAGADRQGTVSFNFPQVTLWQRPLVTIKIGGQLK |
| | | EALLDTGADDTVLEEMSLPGRWKPKMIGGIGGFIKVRQ |
| | | YDQILIEICGHKAIGTVLVGPTPVNIIGRNLLTQIGCT |
| | | LNFPISPIETVPVKLKPGMDGPKVKQWPLTEEKIKALV |
| | | EICTEMEKEGKISKIGPENPYNTPVFAIKKKDSTKWRK |
| | | LVDFRELNKRTQDFWEVQLGIPHPAGLKKKKSVTVLDV |
| | | GDAYFSVPLDEDFRKYTAFTIPSINNETPGIRYQYNVL |
| | | PQGWKGSPAIFQSSMTKILEPFRKQNPDIVIYQYMDDL |
| | | YVGSDLEIGQHRTKIEELRQHLLRWGLTTPDKKHQKEP |
| | | PFLWMGYELHPDKWTVQPIVLPEKDSWTVNDIQKLVGK |
| | | LNWASQIYPGIKVRQLCKLLRGTKALTEVIPLTEEAEL |
| | | ELAENREILKEPVHGVYYDPSKDLIAEIQKQGQGQWTY |
| | | QIYQEPFKNLKTGKYARMRGAHTNDVKQLTEAVQKITT |
| | | ESIVIWGKTPKFKLPIQKETWETWWTEYWQATWIPEWE |
| | | EVNTPPLVKLWYQLEKEPIVGAETFYVDGAANRETKLG |
| | | KAGYVTNRGRQKVVTLTDTTNQKTELQAIYLALQDSGL |
| | | EVNIVTDSQYALGIIQAQPDQSESELVNQIIEQLIKKE |
| | | KVYLAWVPAHKGIGGNEQVDKLVSAGIRKVLFLDGIDK |
| | | AQDEHEKYHSNWRAMASDFNLPPVVAKEIVASCDKCQL |
| | | KGEAMHGQVDCSPGIWQLDCTHLEGKVILVAVHVASGY |
| | | IEAEVIPAETGQETAYFLLKLAGRWPVKIIHTDNGSNF |
| | | TGATVRAACWWAGIKQEFGIPYNPQSQGVVESMNKELK |
| | | KIIGQVRDQAEHLKTAVQMAVFIHNFKRKGGIGGYSAG |
| | | ERIVDIIATDIQTKELQKQITKIQNFRVYYRDSRNPLW |
| | | KGPAKLLWKGEGAVVIQDNSDIKVVPRRKAKIIRDYGK |
| | | QMAGDDCVASRQDED |
| 3 | nef | MGGKWSKSSVIGWPTVRERMRRAEPAADRVGAASRDLE |
| | | KHGAITSSNTAATNAACAWLEAQEEEEVGFPVTPQVPL |
| | | RPMTYKAAVDLSHFLKEKGGLEGLIHSQRRQDILDLWI |
| | | YHTQGYFPDWQNYTPGPGVRYPLTFGWCYKLVPVEPDK |
| | | IEEANKGENTSLLHPVSLHGMDDPEREVLEWRFDSRLA |
| | | FHHVARELHPEYFKNC |

In some embodiments, the plurality of polypeptide segments comprises at least 4 polypeptide segments, e.g., at least 4, 5, 6 or more, polypeptide segments selected from SEQ ID NOs: 4-33, e.g., polypeptide segments identified in Table B.

TABLE B
polypeptide segments in HIV-1 fusion polypeptides

| SEQ ID | | | | Length | |
|---|---|---|---|---|---|
| NO: | Gene | Start | End | (aa) | Sequence |
| 4 | gag | 1 | 53 | 53 | MAARASVLSGGELDRWEKIRLRPGGKKKYRLKH |
| | | | | | IVWASRELERFAVNPGLLET |
| 5 | gag | 1 | 53 | 53 | MAARASILSGGKLDKWEKIRLRPGGRKKYKLKH |
| | | | | | LVWASRELERFALNPGLLET |
| 6 | gag | 147 | 369 | 223 | ISPRTLNAWVKVVEEKAFSPEVIPMFSALSEGA |
| | | | | | TPQDLNTMLNTVGGHQAAMQMLKETINEEAAEW |
| | | | | | DRLHPVHAGPIAPGQMREPRGSDIAGTTSTLQE |
| | | | | | QIGWMTNNPPIPVGEIYKRWIILGLNKIVRMYS |
| | | | | | PTSILDIRQGPKEPFRDYVDRFYKTLRAEQASQ |
| | | | | | EVKNWMTETLLVQNANPDCKTILKALGPAATLE |
| | | | | | EMMTACQGVGGPGHKARVLAEAMSQ |
| 7 | gag | 147 | 369 | 223 | LSPRTLNAWVKVIEEKAFSPEVIPMFTALSEGA |
| | | | | | TPHDLNTMLNTIGGHQAAMQMLKDTINEEAAEW |
| | | | | | DRVHPVHAGPVAPGQMRDPRGSDIAGSTSTLQE |
| | | | | | QIAWMTNNPPIPVGDIYKRWIIMGLNKIVRMYS |
| | | | | | PVSILDIKQGPKEPERDYVDRFYRTLRAEQASQ |
| | | | | | DVKNWMTETLLVQNSNPDCKTILKALGPGATLE |
| | | | | | EMMSACQGVGGPSHKARVLAEAMCQ |
| 8 | pol | 56 | 117 | 62 | FPQITLWQRPLVTIKIGGQLKEALLDTGADDTV |
| | | | | | LEEMNLPGRWKPKMIGGIGGFIKVRQYDQ |
| 9 | pol | 56 | 117 | 62 | LPQITLWQRPIVTIKIGGQIKEALLDTGADDTV |
| | | | | | LEDMNLPGKWKPKMIGGIGGFIKVKQYDQ |
| 10 | pol | 129 | 320 | 192 | GTVLVGPTPVNIIGRNLLTQIGCTLNFPISPIE |
| | | | | | TVPVKLKPGMDGPKVKQWPLTEEKIKALVEICT |
| | | | | | EMEKEGKISKIGPENPYNTPVFAIKKKDSTKWR |
| | | | | | KLVDFRELNKRTQDFWEVQLGIPHPAGLKKKKS |
| | | | | | VTVLDVGDAYFSVPLDKDFRKYTAFTIPSINNE |
| | | | | | TPGIRYQYNVLPQGWKGSPAIFQSSMT |
| 11 | pol | 129 | 320 | 192 | GTVLIGPTPVNIIGRNLLTQLGCTLNFPISPID |
| | | | | | TVPVKLKPGMDGPRVKQWPLTEEKIKALIEICT |
| | | | | | EMEKEGKISRIGPENPYNTPIFAIKKKDGTKWR |
| | | | | | KLVDFRELNKKTQDFWEVQLGIPHPSGLKKKKS |
| | | | | | VTVLDIGDAYFSVPLDKEFRKYTAFTVPSTNNE |
| | | | | | TPGVRYQYNVLPMGWKGSPAIFQCSMT |
| 12 | pol | 367 | 431 | 65 | WGFTTPDKKHQKEPPFLWMGYELHPDKWTVQPI |
| | | | | | VLPEKDSWTVNDIQKLVGKLNWASQIYPGIKV |
| 13 | pol | 367 | 431 | 65 | WGLTTPDKKHQKDPPFLWMGYELHPDRWTVQPI |
| | | | | | ELPEKESWTVNDIQKLIGKLNWASQIYAGIKV |
| 14 | pol | 542 | 606 | 65 | PKFKLPIQKETWETWWTEYWQATWIPEWEFVNT |
| | | | | | PPLVKLWYQLEKEPIVGAETFYVDGAANRETK |
| 15 | pol | 542 | 606 | 65 | PKFRLPIQKETWDTWWIDYWQATWIPEWEFTNT |
| | | | | | PPLVKLWYQLETEPIAGVETFYVDGASNRETK |
| 16 | pol | 586 | 606 | 21 | KEPIVGAETFYVDGAANRETK |
| 17 | pol | 586 | 606 | 21 | TEPIAGVETFYVDGASNRETK |
| 18 | pol | 683 | 708 | 26 | KEKVYLAWVPAHKGIGGNEQVDKLVS |
| 19 | pol | 683 | 708 | 26 | KEKIYLAWVPAHKGIGGNEQIDKLVS |
| 20 | pol | 747 | 827 | 81 | VAKEIVASCDKCQLKGEAMHGQVDCSPGIWQLD |
| | | | | | CTHLEGKIILVAVHVASGYIEAEVIPAETGQET |
| | | | | | AYFLLKLAGRWPVKT |
| 21 | pol | 747 | 827 | 81 | VAKEIVACCDKCQLKGEAIHGQVDCSPGVWQLD |
| | | | | | CTHLEGKVILVAVHVASGYIEAEIIPTETGQET |
| | | | | | AYFILKLAGRWPVTT |
| 22 | pol | 840 | 909 | 69 | TVKAACWWAGIKQEFGIPYNPQSQGVVESMNKE |
| | | | | | LKKIIGQVRDQAEHLKTAVQMAVFIHNFKRKGG |
| | | | | | IGG |
| 23 | pol | 840 | 909 | 69 | AVKAACWWAGVKQEFGIPYNTQSQGVVESMNNE |
| | | | | | LKKIIGQIRDQAEHLKTAVQMAVLIHNFKRKGG |
| | | | | | IGE |
| 24 | pol | 840 | 920 | 81 | TVKAACWWAGIKQEFGIPYNPQSQGVVESMNKE |
| | | | | | LKKIIGQVRDQAEHLKTAVQMAVFIHNFKRKGG |
| | | | | | IGGYSAGERIVDIIA |
| 25 | pol | 840 | 920 | 81 | AVKAACWWAGVKQEFGIPYNTQSQGVVESMNNE |
| | | | | | LKKIIGQIRDQAEHLKTAVQMAVLIHNFKRKGG |
| | | | | | IGEYSAGERIIDIIA |
| 26 | pol | 932 | 1003 | 72 | ITKIQNFRVYYRDSRDPLWKGPAKLLWKGEGAV |
| | | | | | VIQDNSDIKVVPRRKAKIIRDYGKQMAGDDCVA |
| | | | | | SRQDED |
| 27 | pol | 932 | 1003 | 72 | ITKLQNFRVYYRDNRDPLWKGPARLLWKGEGAV |
| | | | | | VIQDNSEIKVVPRRKVKIIRDYGKRMAGDDCVA |
| | | | | | GRQDED |
| 28 | nef | 64 | 76 | 13 | EEVGFPVKPQVPL |
| 29 | nef | 64 | 76 | 13 | EEVGFPVRPQVPL |
| 30 | nef | 64 | 99 | 36 | EEVGFPVKPQVPLRPMTFKGALDLSHFLREKGG |
| | | | | | LEG |
| 31 | nef | 64 | 99 | 36 | EEVGFPVRPQVPLRPMTYKGALDLSHFLKEKGG |
| | | | | | LEG |
| 32 | nef | 117 | 148 | 32 | TQGYFPDWQNYTPGPGTRYPLTFGWCFKLVPV |
| 33 | nef | 117 | 148 | 32 | TQGFFPDWQNYTPGPGIRFPLTFGWCFKLVPL |

In various embodiments, the fusion polypeptide comprises two, three, four, five, six, or more, of the polypeptide segments comprising or consisting of amino acid residues corresponding to Gag 1-53; Gag 147-369; Pol 56-117; Pol 129-320; Pol 367-431 Pol 542-606; Pol 586-606; Pol 683-708, Pol 747-827; Pol 840-909; Pol 840-920; Pol 932-1003; Nef 64-76; Nef 64-99 or Nef 117-148, wherein the Gag, Pol and Nef amino acid position numbers correspond to HIV-1 HXB2 reference sequences, as set forth in SEQ ID NOs: 1, 2 and 3, respectively. In some embodiments, the fusion polypeptide comprises two, three, four, five, six, or more, polypeptide segments selected from SEQ ID NOs: 4-33.

In some embodiments, the fusion polypeptide comprises or consists of the following polypeptide segments, wherein the Gag, Pol and Nef amino acid position numbers correspond to SEQ ID NOs: 1, 2 and 3, respectively:
a) amino acid residues corresponding to: Gag 1-53; Gag 147-369; Pol 683-708 and Nef 117-148;
b amino acid residues corresponding to: Pol 56-117; Pol 129-320; Pol 367-431 and Nef 64-99;
c) amino acid residues corresponding to: Pol 542-606; Pol 747-827; Pol 840-920; Pol 932-1003 and Nef 64-99;
d) amino acid residues corresponding to: Gag 1-53; Gag 147-369; Pol 683-708; Pol 747-827; Pol 840-920 and Nef 117-148;
e) amino acid residues corresponding to: Pol 56-117; Pol 129-320; Pol 367-431; Pol 542-606; Pol 932-1003 and Nef 64-99;
f) amino acid residues corresponding to: Gag 147-369, Pol 586-606, Pol 683-708 and Pol 840-920;
g) amino acid residues corresponding to: Pol 129-320, Pol 747-827, Pol 932-1003 and Nef 64-76; or
h) amino acid residues corresponding to: Gag: 147-369, Pol 747-827, Pol 840-909 and Nef 64-76.

In some embodiments, the fusion polypeptide comprises or consists of the following polypeptide segments:
a) SEQ ID NOs: 4, 6, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 4, 6, 18 and 32, respectively;
b) SEQ ID NOs: 5, 7, 19 and 33, or sequence segments that are at least 80%, 81%, 82%, 97%, 98% or 99% identical to SEQ ID NOs: 5, 7, 19 and 33, respectively;
c) SEQ ID NOs: 8, 10, 12 and 30 or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 10, 12 and 30, respectively;
d) SEQ ID NOs: 9, 11, 13 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 11, 13 and 31, respectively;
e) SEQ ID NOs: 14, 20, 24, 26 and 30, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 14, 20, 24, 26 and 30, respectively;
f) SEQ ID NOs: 15, 21, 25, 27 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 21, 25, 27 and 31, respectively;
g) SEQ ID NOs: 4, 6, 18, 20, 24 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 4, 6, 18, 20, 24 and 32, respectively;
h) SEQ ID NOs: 5, 7, 19, 21, 25 and 33, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 5, 7, 19, 21, 25 and 33, respectively;
i) SEQ ID NOs: 8, 10, 12, 14, 26 and 30, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 10, 12, 14, 26 and 30, respectively;
j) SEQ ID NOs: 9, 11, 13, 15, 27 and 31, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 11, 13, 15, 27 and 31, respectively;
k) SEQ ID NOs: 6, 16, 18 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 16, 18 and 24, respectively;
l) SEQ ID NOs: 7, 17, 19 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 17, 19 and 25, respectively;
m) SEQ ID NOs: 10, 20, 26 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 10, 20, 26 and 28, respectively;
n) SEQ ID NOs: 11, 21, 27 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 21, 27 and 29, respectively;
o) SEQ ID NOs: 6, 20, 22 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 22 and 28, respectively;
p) SEQ ID NOs: 7, 21, 23 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 23 and 29, respectively;
q) SEQ ID NOs: 6, 16, 18 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 16, 18 and 24, respectively;
r) SEQ ID NOs: 7, 17, 19 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 17, 19 and 25, respectively;
S) SEQ ID NOs: 10, 20, 26 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 10, 20, 26 and 28, respectively; or
t) SEQ ID NOs: 11, 21, 27 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 21, 27 and 29, respectively.

Modifications may be made in the structure of the fusion polypeptides and polynucleotides encoding such fusion polypeptides, described herein, and still obtain a functional molecule that encodes a variant or derivative polypeptide with desirable (e.g., immunogenic) characteristics. When it is desired to alter the amino acid sequence of a polypeptide to create an equivalent, or even an improved, variant or portion of a fusion polypeptide described herein, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed fusion polypeptides, or corresponding DNA sequences that encode such fusion polypeptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E. W. and Muller W. (1988) CABIOS 4:11-17; Robinson, E. D. (1971) Comb. Theor 77:105; Santou, N. Nes, M. (1987) Mol. Biol. Evol. 4:406-425; Sneath, P. H. A. and Sokal, R. R. (1973) Numerical Taxonomy-the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

A “polypeptide variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences described herein and evaluating one or more biological activities of the polypeptide as described herein and/or using any of a number of techniques well known in the art. The term “variant” may also refer to any naturally occurring or engineered molecule comprising one or more nucleotide or amino acid mutations.

In some embodiments, the fusion polypeptide comprises or consists of the following polypeptide segments in sequential order, from N-terminus to C-terminus, optionally joined or connected by one or more linkers:
a) SEQ ID NOs: 6, 4, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 4, 18 and 32, respectively;
b) SEQ ID NOs: 7, 5, 33 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 5, 33 and 19, respectively;
c) SEQ ID NOs: 12, 30, 8 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 12, 30, 8 and 10, respectively;
d) SEQ ID NOs: 9, 31, 13 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 31, 13 and 11, respectively;
e) SEQ ID NOs: 14, 26, 20, 30 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 14, 26, 20, 30 and 24, respectively;
f) SEQ ID NOs: 15, 31, 21, 27 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 31, 21, 27 and 25, respectively;
g) SEQ ID NOs: 32, 18, 4 and 6, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 32, 18, 4 and 6, respectively;
h) SEQ ID NOs: 7, 33, 5 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 33, 5 and 19, respectively;
i) SEQ ID NOs: 8, 30, 12 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 30, 12 and 10, respectively;
j) SEQ ID NOs: 13, 31, 9 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 13, 31, 9 and 11, respectively;
k) SEQ ID NOs: 26, 30, 14, 20 and 24, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 30, 14, 20 and 24, respectively;
l) SEQ ID NOs: 31, 27, 15, 25 and 21, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 31, 27, 15, 25 and 21, respectively;
m) SEQ ID NOs: 24, 6, 4, 20, 18 and 32, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 4, 20, 18 and 32, respectively;
n) SEQ ID NOs: 6, 20, 4, 24, 32 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 4, 24, 32 and 18, respectively;
o) SEQ ID NOs: 7, 21, 5, 25, 33 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 5, 25, 33 and 19, respectively;
p) SEQ ID NOs: 8, 30, 14, 12, 26 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 30, 14, 12, 26 and 10, respectively;
q) SEQ ID NOs: 8, 12, 30, 26, 14 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 12, 30, 26, 14 and 10, respectively;
r) SEQ ID NOs: 9, 13, 31, 27, 15 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 9, 13, 31, 27, 15 and 11, respectively;
s) SEQ ID NOs: 20, 32, 24, 4, 6 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 20, 32, 24, 4, 6 and 18, respectively;
t) SEQ ID NOs: 7, 25, 19, 5, 33 and 21, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 25, 19, 5, 33 and 21, respectively;
u) SEQ ID NOs: 26, 30, 12, 14, 8 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 30, 12, 14, 8 and 10, respectively;
v) SEQ ID NOs: 15, 31, 9, 27, 13 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 15, 31, 9, 27, 13 and 11, respectively;
w) SEQ ID NOs: 24, 6, 16 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 16 and 18, respectively;
x) SEQ ID NOs: 7, 19, 17 and 25, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 19, 17 and 25, respectively;
y) SEQ ID NOs: 24, 16, 6 and 18, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 16, 6 and 18, respectively;
z) SEQ ID NOs: 7, 25, 17 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 25, 17 and 19, respectively;
aa) SEQ ID NOs: 26, 20, 10 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 20, 10 and 28, respectively;
bb) SEQ ID NOs: 21, 27, 11 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 21, 27, 11 and 29, respectively;
cc) SEQ ID NOs: 26, 10, 20 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 10, 20 and 28, respectively;
dd) SEQ ID NOs: 11, 27, 21 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 11, 27, 21 and 29, respectively;
ee) SEQ ID NOs: 22, 6, 20 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 6, 20 and 28, respectively;
ff) SEQ ID NOs: 23, 7, 21 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 23, 7, 21 and 29, respectively;
gg) SEQ ID NOs: 22, 20, 6 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 20, 6 and 28, respectively; or
hh) SEQ ID NOs: 7, 21, 23 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 23 and 29, respectively.

Generally, the fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. Generally, the fusion polypeptides do not comprise any polypeptide segments having an amino acid sequence of SEQ ID NOs: 35-47, provided in Table C, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof.

TABLE C
polypeptide segments NOT in present HIV-1
fusion polypeptides

| SEQ ID | | | | |
|---|---|---|---|---|
| NO: | Gene | Start | End | Sequence |
| 35 | Gag | 54 | 146 | EGCRQILGQLQPSLQTGSEELRSLY |
| | | | | NTVATLYCVHQRIEIKDTKEALDKI |
| | | | | EEEQNKSKKKAQQAAADTGHSNQVS |
| | | | | QNYPIVQNIQGQMVHQA |
| 36 | Gag | 370 | 500 | VTNSATIMMQRGNERNQRKIVKCFN |
| | | | | CGKEGHTARNCRAPRKKGCWKCGKE |
| | | | | GHQMKDCTERQANFLGKIWPSYKGR |
| | | | | PGNFLQSRPEPTAPPEESFRSGVET |
| | | | | TTPPQKQEPIDKELYPLTSLRSLFG |
| | | | | NDPSSQ |
| 37 | Pol | 1 | 55 | FFREDLAFLQGKAREFSSEQTRANS |
| | | | | PTRRELQVWGRDNNSPSEAGADRQG |
| | | | | TVSFN |
| 38 | Pol | 118 | 128 | ILIEICGHKAI |
| 39 | Pol | 321 | 366 | KILEPFRKQNPDIVIYQYMDDLYVG |
| | | | | SDLEIGQHRTKIEELRQHLLR |
| 40 | Pol | 432 | 541 | QLCKLLRGTKALTEVIPLTEEAELE |
| | | | | LAENREILKEPVHGVYYDPSKDLIA |
| | | | | EIQKQGQGQWTYQIYQEPFKNLKTG |
| | | | | KYARMRGAHTNDVKQLTEAVQKITT |
| | | | | ESIVIWGKT |
| 41 | Pol | 607 | 682 | GKAGYVTNRGRQKVVTLTDTTNQKT |
| | | | | ELQAIYLALQDSGLEVNIVTDSQYA |
| | | | | LGIIQAQPDQSESELVNQIIEQLIK |
| 42 | Pol | 709 | 746 | AGIRKVLFLDGIDKAQDEHEKYHSN |
| | | | | WRAMASDFNLPPV |
| 43 | Pol | 828 | 839 | IHTDNGSNFTGA |
| 44 | Pol | 921 | 931 | TDIQTKELQKQ |
| 45 | Nef | 1 | 63 | MGGKWSKSSVIGWPTVRERMRRAEP |
| | | | | AADRVGAASRDLEKHGAITSSNTAA |
| | | | | TNAACAWLEAQEE |
| 46 | Nef | 100 | 116 | LIHSQRRQDILDLWIYH |
| 47 | Nef | 149 | 206 | PDKIEEANKGENTSLLHPVSLHGMD |
| | | | | DPEREVLEWRFDSRLAFHHVARELH |
| | | | | PEYFKNC |

With respect to the range of lengths of the individual polypeptide or peptide segments, in various embodiments, each polypeptide segment is at least 25 amino acids in length, and up to about 230 amino acids in length, e.g. from at least 25 amino acids in length up to 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225 or 230 amino acids in length.

With respect to the length of the full-length fusion polypeptide, in various embodiments, in some embodiments, the full-length of the fusion polypeptide comprises at least about 330 amino acids and up to about 550 amino acids, e.g., at least about 330 amino acids and up to about 335, 340, 345, 350, 355, 360, 365, 370, 375, 380, 385, 390, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545 or 550 amino acids in length. In some embodiments, the full-length of the fusion polypeptide is no longer than 550 amino acids, e.g. no longer than 545, 540, 535, 530, 525, 520, 515, 510, 505, 500, 495, 490, 485, 480, 475, 470, 465, 460, 455, 450, 445, 440, 435, 430, 425, 420, 415, 410, 405, 400, 390, 385, 380, 375, 370, 365, 360, 355, 350, 345, 340, 335 or 330 amino acids in length. In various embodiments, in the absence of a signal or leader sequence, the full-length of the fusion polypeptide comprises at least about 330 amino acids and up to about 505 amino acids, e.g., at least about 330 amino acids and up to about 335, 340, 345, 350, 355, 360, 365, 370, 375, 380, 385, 390, 400, 405, 410, 415, 420, 425, 430, 435, 440, 445, 450, 455, 460, 465, 470, 475, 480, 485, 490, 495, 500, 505 amino acids in length.

amino acids in length. In various embodiments, in the absence of a signal or leader sequence, the full-length of the fusion polypeptide is no longer than 505 amino acids, e.g. no longer than 505, 500, 495, 490, 485, 480, 475, 470, 465, 460, 455, 450, 445, 440, 435, 430, 425, 420, 415, 410, 405, 400, 390, 385, 380, 375, 370, 365, 360, 355, 350, 345, 340, 335 or 330 amino acids in length. In various embodiments, in the presence of a signal or leader sequence, the full-length of the fusion polypeptide comprises at least about 350 amino acids and up to about 550 amino acids, e.g., at least about 350 amino acids and up to about 355, 360, 365, 370, 375, 380, 385, 390, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545 or 550 amino acids in length. In various embodiments, in the absence of a signal or leader sequence, the full-length of the fusion polypeptide is no longer than 550 amino acids, e.g. no longer than 545, 540, 535, 530, 525, 520, 515, 510, 505, 500, 495, 490, 485, 480, 475, 470, 465, 460, 455, 450, 445, 440, 435, 430, 425, 420, 415, 410, 405, 400, 390, 385, 380, 375, 370, 365, 360, 355 or 350 amino acids in length. The inclusion of a signal or leader peptide sequence is described in further detail below.

Generally, the fusion polypeptides are immunogenic, in that they are capable of eliciting an immune response in a human, e.g., against HIV-1. In some embodiments, the fusion polypeptides, optionally in combination with one or more additional therapeutic agents, e.g., as described herein, are capable of eliciting a protective or a therapeutically effective immune response in a human against HIV-1, e.g., capable of either preventing HIV-1 infection in an uninfected individual, or in therapeutic contexts, capable of eliciting an immune response sufficient to induce immune mediated control of HIV-1 or eradicate HIV-1 in an infected individual. The immunogenicity of the fusion polypeptides can be evaluated and demonstrated, in in vitro and in vivo assays, as described herein. For example, immunogenicity of the fusion polypeptides can be demonstrated by an in vitro assay, including CD4+ and/or CD8+T-cell activation (e.g., including cytokine expression and target killing assays) or proliferation assays. The T-cells can be activated by exposure to antigen presenting cells (APCs) (such as dendritic cells, e.g., monocyte-derived dendritic cells) that have been transfected with a polynucleotide encoding the fusion polypeptide. Such assays are known in the art and described herein. The immunogenicity of the fusion polypeptides can also be demonstrated in in vivo animal models, for example, by administering to mice, e.g., BALB/c or BL6, or transgenic for one or more human HLA molecules (available from Jackson Laboratories or Taconic), or non-human primates, and evaluating CD4+ and/or CD8+T-cell activation (e.g., including serum cytokine levels) or proliferation. In various embodiments, one, two, three, or more, of each polypeptide segment comprises or consists of one or more predicted T cell epitopes, e.g., as computationally or experimentally determined. In some embodiments, the fusion polypeptide comprises one or more polypeptide segments that bind to or are presented by one or more human HLA class I and/or class II alleles (e.g. 1, 2, 3, 4, 5 or 6 alleles), e.g. within a single subject or amongst multiple subjects.

### Concatenating Polypeptide Segments

As appropriate, the one or more of the polypeptide segments can be directly abutted or fused to an adjacent segment, or can be joined, connected or linked to an adjacent segment by one or more peptide linkers. In various embodiments, the one or more peptide linkers is selected from one or more of a polyalanine linker, a polyglycine linker, a cleavable linker, a flexible linker, a rigid linker, a Nef linking sequence, and combinations thereof, e.g., within a linker or within a full-length fusion polypeptide. Illustrative fusion protein linkers that can be used in the present fusion polypeptides to connect one or more polypeptide segments are described, e.g., in Chen, et al., Adv Drug Deliv Rev. (2013) 65 (10): 1357-1369. In some embodiments, the polyalanine linker comprises or consists of 2 or 3 contiguous alanine residues, e.g. AA, AAA (SEQ ID NO: 48), AAY (SEQ ID NO: 49) or AAX, wherein X is any amino acid (e.g., A, C, D, E, F, G, H, I, K, L, M, N, P, Q, R, S, T, V, Y) (SEQ ID NO: 50). In some embodiments, a polyglycine linker is used, e.g., GGS (SEQ ID NO: 57), GSG (SEQ ID NO: 58) or GGGS (SEQ ID NO: 59). In some embodiments, the cleavable linker is selected from a 2A cleavable peptide. Illustrative 2A cleavable peptides that can be used in the present fusion polypeptides to connect one or more polypeptide segments are described, e.g., in Donnelly, et al., J. Gen. Virol (2001), 82, 1027-1041 and Chng, et al., mAbs (2015) 7:2, 403-412. Illustrative cleavable peptides that can be used to link one or more polypeptide segments include without limitation 2A cleavage sequences (e.g., foot-and-mouth disease virus (F2A), equine rhinitis A virus (E2A), porcine teschovirus-1 (P2A) and Thosea asigna virus (T2A)), and furin recognition/cleavage sequences (e.g. RAKR (SEQ ID NO: 60), REKR (SEQ ID NO: 61), RRKR (SEQ ID NO: 62)). In certain embodiments, a furin recognition/cleavage sequence (e.g., RAKR (SEQ ID NO: 60), REKR (SEQ ID NO: 61), RRKR (SEQ ID NO: 62)) is combined or fused with a 2A cleavable peptide (e.g., foot-and-mouth disease virus (F2A), equine rhinitis A virus (E2A), porcine teschovirus-1 (P2A) and Thosea asigna virus (T2A)) in a single linker. See, e.g., Chng, et al., mAbs (2015) 7:2, 403-412. In various embodiments, the 2A cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67), or comprises or consists of the amino acid sequence of ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67). As appropriate, in certain embodiments, a furin recognition/cleavage sequence can be positioned either at the N-terminus or the C-terminus of a 2A linker. In some embodiments, the cleavable linker comprises or consists of a furin recognition/cleavage site selected from RAKR (SEQ ID NO: 60), REKR (SEQ ID NO: 61) and RRKR (SEQ ID NO: 62). REKR (SEQ ID NO: 61) is a naturally occurring cleavable linker in HIV and SIV envelope glycoprotein precursor (Bahbouhi, et al., Biochem. J. (2002) 366, 863-872). Illustrative linkers that can be used to link or connect one or more polypeptide segments in a fusion polypeptide are provided in Table D.

TABLE D
illustrative linkers

| SEQ | | |
|---|---|---|
| ID | | |
| NO: | NAME | SEQUENCE |
| | poly-alanine (2) | AA |
| 48 | poly-alanine (3) | AAA |
| 49 | poly-alanine-Tyr | AAY |
| 50 | poly-alanine-Xaa | AAX (X = any |
| | | amino acid) |
| 51 | Gln-Glu-Glu | QEE |
| | Glu-Glu | EE |
| | Isoleucine | I |
| | Lysine | K |
| | Leu-Ile | LI |
| 52 | Lys-Ile-Leu | KIL |
| 53 | Leu-Ile-Lys | LIK |
| 54 | Pro-Pro-Val | PPV |
| 55 | Ser-Glu-Gly | SEG |
| | poly-glycine (2) | GG |
| 57 | poly-glycine | GGS |
| 58 | poly-glycine | GSG |
| 59 | Gly3Ser | GGGS |
| 60 | furin recognition site | RAKR |
| 61 | furin recognition site | REKR |
| 62 | furin recognition site | RRKR |
| 63 | P2A | ATNFSLLKQAGDVEENPGP |
| 64 | F2A | APVKQTLNFDLLKLAGDVE |
| | | SNPGP |
| 65 | F2A + N-terminal | RAKRAPVKQTLNFDLLKLAG |
| | furin recognition site | DVESNPGP |
| 66 | E2A | QCTNYALLKLAGDVESNPGP |
| 67 | T2A | EGRGSLLTCGDVEENPGP |

Illustrative fusion polypeptides, without signal sequences, which have been designed and assembled according to the herein described methods, are provided in Table E. Table E discloses “AAA” as SEQ ID NO: 48, “LIK” as SEQ ID NO: 53, “AAY” as SEQ ID NO: 49, “SEG” as SEQ ID NO: 55, “QEE” as SEQ ID NO: 51, “KIL” as SEQ ID NO: 52 and “PPV” as SEQ ID NO: 54.

In some embodiments, the fusion polypeptide comprises or consists of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the fusion polypeptide comprises or consists of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223. In some embodiments, the fusion polypeptide comprises or consists of an amino acid sequence of SEQ ID NO: 82, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 82. In some embodiments, the fusion polypeptide comprises or consists of an amino acid sequence of SEQ ID NO: 83, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 83. In some embodiments, the fusion polypeptide comprises or consists of an amino acid sequence of SEQ ID NO: 85, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 85. In some embodiments, the fusion polypeptide comprises or consists of an amino acid sequence of SEQ ID NO: 86, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 86. In some embodiments, the fusion polypeptide comprises or consists of an amino acid sequence of SEQ ID NO: 87, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 87. In some embodiments, the fusion polypeptide comprises or consists of an amino acid sequence of SEQ ID NO: 98, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 98. In some embodiments, the fusion polypeptide comprises or consists of an amino acid sequence of SEQ ID NO: 99, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 99. In some embodiments, the fusion polypeptide comprises or consists of an amino acid sequence of SEQ ID NO: 100, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 100. In some embodiments, the fusion polypeptide comprises or consists of an amino acid sequence of SEQ ID NO: 101, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 101. In some embodiments, the fusion polypeptide comprises or consists of an amino acid sequence of SEQ ID NO: 209, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209. In some embodiments, the fusion polypeptide comprises or consists of an amino acid sequence of SEQ ID NO: 222, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222. In some embodiments, the fusion polypeptide comprises or consists of an amino acid sequence of SEQ ID NO: 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In some embodiments, the fusion polypeptide comprises or consists of an amino acid sequence of SEQ ID NO: 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 227. As appropriate or desired, the fusion polypeptide can have an N-terminal methionine residue.

TABLE E
Illustrative Fusion Polypeptides

| | Polypeptide | | |
|---|---|---|---|
| SEQ | segments | | |
| ID | (N-term to | Length | |
| NO: | C-term) | (aa) | Amino Acid Sequence |
| 70 | Gag: 147-369 | 337 | ISPRTLNAWVKVVEEKAFSPEVIPMFSALSEGATPQDLNTMLNTVGGHQAAMQMLKETINE |
| | Gag: 1-53 | | EAAEWDRLHPVHAGPIAPGQMREPRGSDIAGTTSTLQEQIGWMTNNPPIPVGEIYKRWIIL |
| | AAA | | GLNKIVRMYSPTSILDIRQGPKEPFRDYVDRFYKTLRAEQASQEVKNWMTETLLVQNANPD |
| | Pol: 683-708 | | CKTILKALGPAATLEEMMTACQGVGGPGHKARVLAEAMSQMAARASVLSGGELDRWEKIRL |
| | Nef: 117-148 | | RPGGKKKYRLKHIVWASRELERFAVNPGLLETAAAKEKVYLAWVPAHKGIGGNEQVDKLVS |
| | | | TQGYFPDWQNYTPGPGTRYPLTFGWCFKLVPV |
| 71 | Gag: 147-369 | 339 | LSPRTLNAWVKVIEEKAFSPEVIPMFTALSEGATPHDLNTMLNTIGGHQAAMQMLKDTINE |
| | AA | | EAAEWDRVHPVHAGPVAPGQMRDPRGSDIAGSTSTLQEQIAWMTNNPPIPVGDIYKRWIIM |
| | Gag: 1-53 | | GLNKIVRMYSPVSILDIKQGPKEPFRDYVDRFYRTLRAEQASQDVKNWMTETLLVQNSNPD |
| | Nef: 117-148 | | CKTILKALGPGATLEEMMSACQGVGGPSHKARVLAEAMCQAAMAARASILSGGKLDKWEKI |
| | LIK | | RLRPGGRKKYKLKHLVWASRELERFALNPGLLETTQGFFPDWQNYTPGPGIRFPLTFGWCF |
| | Pol: 683-708 | | KLVPLLIKKEKIYLAWVPAHKGIGGNEQIDKLVS |
| 72 | Pol: 367-431 | 360 | WGFTTPDKKHQKEPPFLWMGYELHPDKWTVQPIVLPEKDSWTVNDIQKLVGKLNWASQIYP |
| | AA | | GIKVAAQEEEEVGFPVKPQVPLRPMTFKGALDLSHFLREKGGLEGFPQITLWQRPLVTIKI |
| | QEE | | GGQLKEALLDTGADDTVLEEMNLPGRWKPKMIGGIGGFIKVRQYDQGTVLVGPTPVNIIGR |
| | Nef: 64-99 | | NLLTQIGCTLNFPISPIETVPVKLKPGMDGPKVKQWPLTEEKIKALVEICTEMEKEGKISK |
| | Pol: 56-117 | | IGPENPYNTPVFAIKKKDSTKWRKLVDFRELNKRTQDFWEVQLGIPHPAGLKKKKSVTVLD |
| | Pol: 129-320 | | VGDAYFSVPLDKDFRKYTAFTIPSINNETPGIRYQYNVLPQGWKGSPAIFQSSMT |
| 73 | Pol: 56-117 | 356 | LPQITLWQRPIVTIKIGGQIKEALLDTGADDTVLEDMNLPGKWKPKMIGGIGGFIKVKQYD |
| | Nef: 64-99 | | QEEVGFPVRPQVPLRPMTYKGALDLSHFLKEKGGLEGWGLTTPDKKHQKDPPFLWMGYELH |
| | Pol: 367-431 | | PDRWTVQPIELPEKESWTVNDIQKLIGKLNWASQIYAGIKVKGTVLIGPTPVNIIGRNLLT |
| | K | | QLGCTLNFPISPIDTVPVKLKPGMDGPRVKQWPLTEEKIKALIEICTEMEKEGKISRIGPE |
| | Pol: 129-320 | | NPYNTPIFAIKKKDGTKWRKLVDFRELNKKTQDFWEVQLGIPHPSGLKKKKSVTVLDIGDA |
| | | | YFSVPLDKEFRKYTAFTVPSTNNETPGVRYQYNVLPMGWKGSPAIFQCSMT |
| 74 | Pol: 542-606 | 335 | PKFKLPIQKETWETWWTEYWQATWIPEWEFVNTPPLVKLWYQLEKEPIVGAETFYVDGAAN |
| | Pol: 932-1003 | | RETKITKIQNFRVYYRDSRDPLWKGPAKLLWKGEGAVVIQDNSDIKVVPRRKAKIIRDYGK |
| | Pol: 747-827 | | QMAGDDCVASRQDEDVAKEIVASCDKCQLKGEAMHGQVDCSPGIWQLDCTHLEGKIILVAV |
| | Nef: 64-99 | | HVASGYIEAEVIPAETGQETAYFLLKLAGRWPVKTEEVGFPVKPQVPLRPMTFKGALDLSH |
| | Pol: 840-920 | | FLREKGGLEGTVKAACWWAGIKQEFGIPYNPQSQGVVESMNKELKKIIGQVRDQAEHLKTA |
| | | | VQMAVFIHNFKRKGGIGGYSAGERIVDIIA |
| 75 | Pol: 542-606 | 335 | PKFRLPIQKETWDTWWTDYWQATWIPEWEFTNTPPLVKLWYQLETEPIAGVETFYVDGASN |
| | Nef: 64-99 | | RETKEEVGFPVRPQVPLRPMTYKGALDLSHFLKEKGGLEGVAKEIVACCDKCQLKGEAIHG |
| | Pol: 747-827 | | QVDCSPGVWQLDCTHLEGKVILVAVHVASGYIEAEIIPTETGQETAYFILKLAGRWPVTTI |
| | Pol: 932-1003 | | TKLQNFRVYYRDNRDPLWKGPARLLWKGEGAVVIQDNSEIKVVPRRKVKIIRDYGKRMAGD |
| | Pol: 840-920 | | DCVAGRQDEDAVKAACWWAGVKQEFGIPYNTQSQGVVESMNNELKKIIGQIRDQAEHLKTA |
| | | | VQMAVLIHNFKRKGGIGEYSAGERIIDIIA |
| 76 | Nef: 117-148 | 340 | TQGYFPDWQNYTPGPGTRYPLTFGWCFKLVPVKEKVYLAWVPAHKGIGGNEQVDKLVSAAY |
| | Pol: 683-708 | | MAARASVLSGGELDRWEKIRLRPGGKKKYRLKHIVWASRELERFAVNPGLLETSEGISPRT |
| | AY | | LNAWVKVVEEKAFSPEVIPMFSALSEGATPQDLNTMLNTVGGHQAAMQMLKETINEEAAEW |
| | Gag: 1-53 | | DRLHPVHAGPIAPGQMREPRGSDIAGTTSTLQEQIGWMTNNPPIPVGEIYKRWIILGLNKI |
| | SEG | | VRMYSPTSILDIRQGPKEPFRDYVDRFYKTLRAEQASQEVKNWMTETLLVQNANPDCKTIL |
| | Gag: 147-369 | | KALGPAATLEEMMTACQGVGGPGHKARVLAEAMSQ |
| 77 | Gag: 147-369 | 334 | LSPRTLNAWVKVIEEKAFSPEVIPMFTALSEGATPHDLNTMLNTIGGHQAAMQMLKDTINE |
| | Nef: 117-148 | | EAAEWDRVHPVHAGPVAPGQMRDPRGSDIAGSTSTLQEQIAWMTNNPPIPVGDIYKRWIIM |
| | Gag: 1-53 | | GLNKIVRMYSPVSILDIKQGPKEPFRDYVDRFYRTLRAEQASQDVKNWMTETLLVQNSNPD |
| | Pol: 683-708 | | CKTILKALGPGATLEEMMSACQGVGGPSHKARVLAEAMCQTQGFFPDWQNYTPGPGIRFPL |
| | | | TFGWCFKLVPLMAARASILSGGKLDKWEKIRLRPGGRKKYKLKHLVWASRELERFALNPGL |
| | | | LETKEKIYLAWVPAHKGIGGNEQIDKLVS |
| 78 | Pol: 56-117 | 360 | FPQITLWQRPLVTIKIGGQLKEALLDTGADDTVLEEMNLPGRWKPKMIGGIGGFIKVRQYD |
| | AAY | | QAAYEEVGFPVKPQVPLRPMTFKGALDLSHFLREKGGLEGAAWGFTTPDKKHQKEPPFLWM |
| | Nef: 64-99 | | GYELHPDKWTVQPIVLPEKDSWTVNDIQKLVGKLNWASQIYPGIKVGTVLVGPTPVNIIGR |
| | AA | | NLLTQIGCTLNFPISPIETVPVKLKPGMDGPKVKQWPLTEEKIKALVEICTEMEKEGKISK |
| | Pol: 367-431 | | IGPENPYNTPVFAIKKKDSTKWRKLVDFRELNKRTQDFWEVQLGIPHPAGLKKKKSVTVLD |
| | Pol: 129-320 | | VGDAYFSVPLDKDFRKYTAFTIPSINNETPGIRYQYNVLPQGWKGSPAIFQSSMT |
| 79 | Pol: 367-431 | 358 | WGLTTPDKKHQKDPPFLWMGYELHPDRWTVQPIELPEKESWTVNDIQKLIGKLNWASQIYA |
| | Nef: 64-99 | | GIKVEEVGFPVRPQVPLRPMTYKGALDLSHFLKEKGGLEGLPQITLWQRPIVTIKIGGQIK |
| | Pol: 56-117 | | EALLDTGADDTVLEDMNLPGKWKPKMIGGIGGFIKVKQYDQAAAGTVLIGPTPVNIIGRNL |
| | AAA | | LTQLGCTLNFPISPIDTVPVKLKPGMDGPRVKQWPLTEEKIKALIEICTEMEKEGKISRIG |
| | Pol: 129-320 | | PENPYNTPIFAIKKKDGTKWRKLVDFRELNKKTQDFWEVQLGIPHPSGLKKKKSVTVLDIG |
| | | | DAYFSVPLDKEFRKYTAFTVPSTNNETPGVRYQYNVLPMGWKGSPAIFQCSMT |
| 80 | Pol: 932-1003 | 342 | ITKIQNFRVYYRDSRDPLWKGPAKLLWKGEGAVVIQDNSDIKVVPRRKAKIIRDYGKQMAG |
| | AAY | | DDCVASRQDEDAAYEEEEVGFPVKPQVPLRPMTFKGALDLSHFLREKGGLEGLIPKFKLPI |
| | EE | | QKETWETWWTEYWQATWIPEWEFVNTPPLVKLWYQLEKEPIVGAETFYVDGAANRETKVAK |
| | Nef: 64-99 | | EIVASCDKCQLKGEAMHGQVDCSPGIWQLDCTHLEGKIILVAVHVASGYIEAEVIPAETGQ |
| | LI | | ETAYFLLKLAGRWPVKTTVKAACWWAGIKQEFGIPYNPQSQGVVESMNKELKKIIGQVRDQ |
| | Pol: 542-606 | | AEHLKTAVQMAVFIHNFKRKGGIGGYSAGERIVDIIA |
| | Pol: 747-827 | | |
| | Pol: 840-920 | | |
| 81 | Nef: 64-99 | 335 | EEVGFPVRPQVPLRPMTYKGALDLSHFLKEKGGLEGITKLQNFRVYYRDNRDPLWKGPARL |
| | Pol: 932-1003 | | LWKGEGAVVIQDNSEIKVVPRRKVKIIRDYGKRMAGDDCVAGRQDEDPKFRLPIQKETWDT |
| | Pol: 542-606 | | WWITYWQATWIPEWEFTNTPPLVKLWYQLETEPIAGVETFYVDGASNRETKAVKAACWWAG |
| | Pol: 840-920 | | VKQEFGIPYNTQSQGVVESMNNELKKIIGQIRDQAEHLKTAVQMAVLIHNFKRKGGIGEYS |
| | Pol: 747-827 | | AGERIIDIIAVAKEIVACCDKCQLKGEAIHGQVDCSPGVWQLDCTHLEGKVILVAVHVASG |
| | | | YIEAEIIPTETGQETAYFILKLAGRWPVTT |
| 82 | Pol: 840-920 | 353 | TVKAACWWAGIKQEFGIPYNPQSQGVVESMNKELKKIIGQVRDQAEHLKTAVQMAVFIHNF |
| | Gag: 147-369 | | KRKGGIGGYSAGERIVDIIAISPRTLNAWVKVVEEKAFSPEVIPMFSALSEGATPQDLNTM |
| | Pol: 586-606 | | LNTVGGHQAAMQMLKETINEEAAEWDRLHPVHAGPIAPGQMREPRGSDIAGTTSTLQEQIG |
| | AA | | WMTNNPPIPVGEIYKRWIILGLNKIVRMYSPTSILDIRQGPKEPFRDYVDRFYKTLRAEQA |
| | Pol: 683-708 | | SQEVKNWMTETLLVQNANPDCKTILKALGPAATLEEMMTACQGVGGPGHKARVLAEAMSQK |
| | | | EPIVGAETFYVDGAANRETKAAKEKVYLAWVPAHKGIGGNEQVDKLVS |
| 83 | Gag: 147-369 | 351 | LSPRTLNAWVKVIEEKAFSPEVIPMFTALSEGATPHDLNTMLNTIGGHQAAMQMLKDTINE |
| | Pol: 683-708 | | EAAEWDRVHPVHAGPVAPGQMRDPRGSDIAGSTSTLQEQIAWMTNNPPIPVGDIYKRWIIM |
| | Pol: 586-606 | | GLNKIVRMYSPVSILDIKQGPKEPFRDYVDRFYRTLRAEQASQDVKNWMTETLLVQNSNPD |
| | Pol: 840-920 | | CKTILKALGPGATLEEMMSACQGVGGPSHKARVLAEAMCQKEKIYLAWVPAHKGIGGNEQI |
| | | | DKLVSTEPIAGVETFYVDGASNRETKAVKAACWWAGVKQEFGIPYNTQSQGVVESMNNELK |
| | | | KIIGQIRDQAEHLKTAVQMAVLIHNFKRKGGIGEYSAGERIIDIIA |
| 84 | Pol: 840-920 | 358 | TVKAACWWAGIKQEFGIPYNPQSQGVVESMNKELKKIIGQVRDQAEHLKTAVQMAVFIHNF |
| | Pol: 586-606 | | KRKGGIGGYSAGERIVDIIAKEPIVGAETFYVDGAANRETKAAAISPRTLNAWVKVVEEKA |
| | AAA | | FSPEVIPMFSALSEGATPQDLNTMLNTVGGHQAAMQMLKETINEEAAEWDRLHPVHAGPIA |
| | Gag: 147-369 | | PGQMREPRGSDIAGTTSTLQEQIGWMTNNPPIPVGEIYKRWIILGLNKIVRMYSPTSILDI |
| | AAA | | RQGPKEPFRDYVDRFYKTLRAEQASQEVKNWMTETLLVQNANPDCKTILKALGPAATLEEM |
| | Pol: 683-708 | | MTACQGVGGPGHKARVLAEAMSQAAAKEKVYLAWVPAHKGIGGNEQVDKLVSL |
| 85 | Gag: 147-369 | 353 | LSPRTLNAWVKVIEEKAFSPEVIPMFTALSEGATPHDLNTMLNTIGGHQAAMQMLKDTINE |
| | Pol: 840-920 | | EAAEWDRVHPVHAGPVAPGQMRDPRGSDIAGSTSTLQEQIAWMTNNPPIPVGDIYKRWIIM |
| | Pol: 586-606 | | GLNKIVRMYSPVSILDIKQGPKEPFRDYVDRFYRTLRAEQASQDVKNWMTETLLVQNSNPD |
| | AA | | CKTILKALGPGATLEEMMSACQGVGGPSHKARVLAEAMCQAVKAACWWAGVKQEFGIPYNT |
| | Pol: 683-708 | | QSQGVVESMNNELKKIIGQIRDQAEHLKTAVQMAVLIHNFKRKGGIGEYSAGERIIDIIAT |
| | | | EPIAGVETFYVDGASNRETKAAKEKIYLAWVPAHKGIGGNEQIDKLVS |
| 86 | Pol: 932-1003 | 361 | ITKIQNFRVYYRDSRDPLWKGPAKLLWKGEGAVVIQDNSDIKVVPRRKAKIIRDYGKQMAG |
| | Pol: 747-827 | | DDCVASRQDEDVAKEIVASCDKCQLKGEAMHGQVDCSPGIWQLDCTHLEGKIILVAVHVAS |
| | Pol: 129-320 | | GYIEAEVIPAETGQETAYFLLKLAGRWPVKTGTVLVGPTPVNIIGRNLLTQIGCTLNFPIS |
| | QEE | | PIETVPVKLKPGMDGPKVKQWPLTEEKIKALVEICTEMEKEGKISKIGPENPYNTPVFAIK |
| | Nef: 64-76 | | KKDSTKWRKLVDFRELNKRTQDFWEVQLGIPHPAGLKKKKSVTVLDVGDAYFSVPLDKDFR |
| | | | KYTAFTIPSINNETPGIRYQYNVLPQGWKGSPAIFQSSMTQEEEEVGFPVKPQVPL |
| 87 | Pol: 747-827 | 364 | VAKEIVACCDKCQLKGEAIHGQVDCSPGVWQLDCTHLEGKVILVAVHVASGYIEAEIIPTE |
| | Pol: 932-1003 | | TGQETAYFILKLAGRWPVTTITKLQNFRVYYRDNRDPLWKGPARLLWKGEGAVVIQDNSEI |
| | Pol: 129-320 | | KVVPRRKVKIIRDYGKRMAGDDCVAGRQDEDGTVLIGPTPVNIIGRNLLTQLGCTLNFPIS |
| | KIL | | PIDTVPVKLKPGMDGPRVKQWPLTEEKIKALIEICTEMEKEGKISRIGPENPYNTPIFAIK |
| | QEE | | KKDGTKWRKLVDFRELNKKTQDFWEVQLGIPHPSGLKKKKSVTVLDIGDAYFSVPLDKEFR |
| | Nef: 64-76 | | KYTAFTVPSTNNETPGVRYQYNVLPMGWKGSPAIFQCSMTKILQEEEEVGFPVRPQVPL |
| 88 | Pol: 932-1003 | 365 | ITKIQNFRVYYRDSRDPLWKGPAKLLWKGEGAVVIQDNSDIKVVPRRKAKIIRDYGKQMAG |
| | AAA | | DDCVASRQDEDAAAIGTVLVGPTPVNIIGRNLLTQIGCTLNFPISPIETVPVKLKPGMDGP |
| | I | | KVKQWPLTEEKIKALVEICTEMEKEGKISKIGPENPYNTPVFAIKKKDSTKWRKLVDFREL |
| | Pol: 129-320 | | NKRTQDFWEVQLGIPHPAGLKKKKSVTVLDVGDAYFSVPLDKDFRKYTAFTIPSINNETPG |
| | Pol: 747-827 | | IRYQYNVLPQGWKGSPAIFQSSMTVAKEIVASCDKCQLKGEAMHGQVDCSPGIWQLDCTHL |
| | QEE | | EGKIILVAVHVASGYIEAEVIPAETGQETAYFLLKLAGRWPVKTQEEEEVGFPVKPQVPL |
| | Nef: 64-76 | | |
| 89 | Pol: 129-320 | 363 | GTVLIGPTPVNIIGRNLLTQLGCTLNFPISPIDTVPVKLKPGMDGPRVKQWPLTEEKIKAL |
| | Pol: 932-1003 | | IEICTEMEKEGKISRIGPENPYNTPIFAIKKKDGTKWRKLVDFRELNKKTQDFWEVQLGIP |
| | Pol: 747-827 | | HPSGLKKKKSVTVLDIGDAYFSVPLDKEFRKYTAFTVPSTNNETPGVRYQYNVLPMGWKGS |
| | AA | | PAIFQCSMTITKLQNFRVYYRDNRDPLWKGPARLLWKGEGAVVIQDNSEIKVVPRRKVKII |
| | QEE | | RDYGKRMAGDDCVAGRQDEDVAKEIVACCDKCQLKGEAIHGQVDCSPGVWQLDCTHLEGKV |
| | Nef: 64-76 | | ILVAVHVASGYIEAEIIPTETGQETAYFILKLAGRWPVTTAAQEEEEVGFPVRPQVPL |
| 90 | Pol: 840-909 | 391 | TVKAACWWAGIKQEFGIPYNPQSQGVVESMNKELKKIIGQVRDQAEHLKTAVQMAVFIHNF |
| | AA | | KRKGGIGGAAISPRTLNAWVKVVEEKAFSPEVIPMFSALSEGATPQDLNTMLNTVGGHQAA |
| | Gag: 147-369 | | MQMLKETINEEAAEWDRLHPVHAGPIAPGQMREPRGSDIAGTTSTLQEQIGWMTNNPPIPV |
| | Pol: 747-827 | | GEIYKRWIILGLNKIVRMYSPTSILDIRQGPKEPFRDYVDRFYKTLRAEQASQEVKNWMTE |
| | QEE | | TLLVQNANPDCKTILKALGPAATLEEMMTACQGVGGPGHKARVLAEAMSQVAKEIVASCDK |
| | Nef: 64-76 | | CQLKGEAMHGQVDCSPGIWQLDCTHLEGKIILVAVHVASGYIEAEVIPAETGQETAYFLLK |
| | | | LAGRWPVKTQEEEEVGFPVKPQVPL |
| 91 | Pol: 840-909 | 393 | AVKAACWWAGVKQEFGIPYNTQSQGVVESMNNELKKIIGQIRDQAEHLKTAVQMAVLIHNF |
| | Gag: 147-369 | | KRKGGIGELSPRTLNAWVKVIEEKAFSPEVIPMFTALSEGATPHDLNTMLNTIGGHQAAMQ |
| | VT | | MLKDTINEEAAEWDRVHPVHAGPVAPGQMRDPRGSDIAGSTSTLQEQIAWMTNNPPIPVGD |
| | Pol: 747-827 | | IYKRWIIMGLNKIVRMYSPVSILDIKQGPKEPFRDYVDRFYRTLRAEQASQDVKNWMTETL |
| | AA | | LVQNSNPDCKTILKALGPGATLEEMMSACQGVGGPSHKARVLAEAMCQVTVAKEIVACCDK |
| | QEE | | CQLKGEAIHGQVDCSPGVWQLDCTHLEGKVILVAVHVASGYIEAEIIPTETGQETAYFILK |
| | Nef: 64-76 | | LAGRWPVTTAAQEEEEVGFPVRPQVPL |
| 92 | Pol: 840-909 | 391 | TVKAACWWAGIKQEFGIPYNPQSQGVVESMNKELKKIIGQVRDQAEHLKTAVQMAVFIHNF |
| | Pol: 747-827 | | KRKGGIGGVAKEIVASCDKCQLKGEAMHGQVDCSPGIWQLDCTHLEGKIILVAVHVASGYI |
| | AA | | EAEVIPAETGQETAYFLLKLAGRWPVKTAAISPRTLNAWVKVVEEKAFSPEVIPMFSALSE |
| | Gag: 147-369 | | GATPQDLNTMLNTVGGHQAAMQMLKETINEEAAEWDRLHPVHAGPIAPGQMREPRGSDIAG |
| | QEE | | TTSTLQEQIGWMTNNPPIPVGEIYKRWIILGLNKIVRMYSPTSILDIRQGPKEPFRDYVDR |
| | Nef: 64-76 | | FYKTLRAEQASQEVKNWMTETLLVQNANPDCKTILKALGPAATLEEMMTACQGVGGPGHKA |
| | | | RVLAEAMSQQEEEEVGFPVKPQVPL |
| 93 | Gag: 147-369 | 394 | LSPRTLNAWVKVIEEKAFSPEVIPMFTALSEGATPHDLNTMLNTIGGHQAAMQMLKDTINE |
| | PV | | EAAEWDRVHPVHAGPVAPGQMRDPRGSDIAGSTSTLQEQIAWMTNNPPIPVGDIYKRWIIM |
| | Pol: 747-827 | | GLNKIVRMYSPVSILDIKQGPKEPFRDYVDRFYRTLRAEQASQDVKNWMTETLLVQNSNPD |
| | AAY | | CKTILKALGPGATLEEMMSACQGVGGPSHKARVLAEAMCQPVVAKEIVACCDKCQLKGEAI |
| | Pol: 840-909 | | HGQVDCSPGVWQLDCTHLEGKVILVAVHVASGYIEAEIIPTETGQETAYFILKLAGRWPVT |
| | QEE | | TAAYAVKAACWWAGVKQEFGIPYNTQSQGVVESMNNELKKIIGQIRDQAEHLKTAVQMAVL |
| | Nef: 64-76 | | IHNFKRKGGIGEQEEEEVGFPVRPQVPL |
| 94 | Pol: 840-920 | 499 | TVKAACWWAGIKQEFGIPYNPQSQGVVESMNKELKKIIGQVRDQAEHLKTAVQMAVFIHNF |
| | Gag: 147-369 | | KRKGGIGGYSAGERIVDIIAISPRTLNAWVKVVEEKAFSPEVIPMFSALSEGATPQDLNTM |
| | Gag: 1-53 | | LNTVGGHQAAMQMLKETINEEAAEWDRLHPVHAGPIAPGQMREPRGSDIAGTTSTLQEQIG |
| | PPV | | WMTNNPPIPVGEIYKRWIILGLNKIVRMYSPTSILDIRQGPKEPFRDYVDRFYKTLRAEQA |
| | Pol: 747-827 | | SQEVKNWMTETLLVQNANPDCKTILKALGPAATLEEMMTACQGVGGPGHKARVLAEAMSQM |
| | Pol: 683-708 | | AARASVLSGGELDRWEKIRLRPGGKKKYRLKHIVWASRELERFAVNPGLLETPPVVAKEIV |
| | Nef: 117-148 | | ASCDKCQLKGEAMHGQVDCSPGIWQLDCTHLEGKIILVAVHVASGYIEAEVIPAETGQETA |
| | | | YFLLKLAGRWPVKTKEKVYLAWVPAHKGIGGNEQVDKLVSTQGYFPDWQNYTPGPGTRYPL |
| | | | TFGWCFKLVPV |
| 95 | Gag: 147-369 | 501 | LSPRTLNAWVKVIEEKAFSPEVIPMFTALSEGATPHDLNTMLNTIGGHQAAMQMLKDTINE |
| | Pol: 747-827 | | EAAEWDRVHPVHAGPVAPGQMRDPRGSDIAGSTSTLQEQIAWMTNNPPIPVGDIYKRWIIM |
| | Gag: 1-53 | | GLNKIVRMYSPVSILDIKQGPKEPFRDYVDRFYRTLRAEQASQDVKNWMTETLLVQNSNPD |
| | AA | | CKTILKALGPGATLEEMMSACQGVGGPSHKARVLAEAMCQVAKEIVACCDKCQLKGEAIHG |
| | Pol: 840-920 | | QVDCSPGVWQLDCTHLEGKVILVAVHVASGYIEAEIIPTETGQETAYFILKLAGRWPVTTM |
| | Nef: 117-148 | | AARASILSGGKLDKWEKIRLRPGGRKKYKLKHLVWASRELERFALNPGLLETAAAVKAACW |
| | LIK | | WAGVKQEFGIPYNTQSQGVVESMNNELKKIIGQIRDQAEHLKTAVQMAVLIHNFKRKGGIG |
| | Pol: 683-708 | | EYSAGERIIDIIATQGFFPDWQNYTPGPGIRFPLTFGWCFKLVPLLIKKEKIYLAWVPAHK |
| | | | GIGGNEQIDKLVS |
| 220 | Gag: 147-369 | 501 | ISPRTLNAWVKVVEEKAFSPEVIPMFSALSEGATPQDLNTMLNTVGGHQAAMQMLKETINE |
| | Pol: 747-827 | | EAAEWDRLHPVHAGPIAPGQMREPRGSDIAGTTSTLQEQIGWMTNNPPIPVGEIYKRWIIL |
| | Gag: 1-53 | | GLNKIVRMYSPTSILDIRQGPKEPFRDYVDRFYKTLRAEQASQEVKNWMTETLLVQNANPD |
| | AA | | CKTILKALGPAATLEEMMTACQGVGGPGHKARVLAEAMSQPPVVAKEIVASCDKCQLKGEA |
| | Pol: 840-920 | | MHGQVDCSPGIWQLDCTHLEGKIILVAVHVASGYIEAEVIPAETGQETAYFLLKLAGRWPV |
| | Nef: 117-148 | | KTMAARASVLSGGELDRWEKIRLRPGGKKKYRLKHIVWASRELERFAVNPGLLETAATVKA |
| | LIK | | ACWWAGIKQEFGIPYNPQSQGVVESMNKELKKIIGQVRDQAEHLKTAVQMAVFIHNFKRKG |
| | Pol: 683-708 | | GIGGYSAGERIVDIIATQGYFPDWQNYTPGPGTRYPLTFGWCFKLVPVKEKVYLAWVPAHK |
| | | | GIGGNEQVDKLVS |
| 96 | Pol: 56-117 | 494 | FPQITLWQRPLVTIKIGGQLKEALLDTGADDTVLEEMNLPGRWKPKMIGGIGGFIKVRQYD |
| | Nef: 64-99 | | QEEVGFPVKPQVPLRPMTFKGALDLSHFLREKGGLEGLIPKFKLPIQKETWETWWTEYWQA |
| | LI | | TWIPEWEFVNTPPLVKLWYQLEKEPIVGAETFYVDGAANRETKWGFTTPDKKHQKEPPFLW |
| | Pol: 542-606 | | MGYELHPDKWTVQPIVLPEKDSWTVNDIQKLVGKLNWASQIYPGIKVITKIQNFRVYYRDS |
| | Pol: 367-431 | | RDPLWKGPAKLLWKGEGAVVIQDNSDIKVVPRRKAKIIRDYGKQMAGDDCVASRQDEDGTV |
| | Pol: 932-1003 | | LVGPTPVNIIGRNLLTQIGCTLNFPISPIETVPVKLKPGMDGPKVKQWPLTEEKIKALVEI |
| | Pol: 129-320 | | CTEMEKEGKISKIGPENPYNTPVFAIKKKDSTKWRKLVDFRELNKRTQDFWEVQLGIPHPA |
| | | | GLKKKKSVTVLDVGDAYFSVPLDKDFRKYTAFTIPSINNETPGIRYQYNVLPQGWKGSPAI |
| | | | FQSSMT |
| 97 | Pol: 56-117 | 499 | LPQITLWQRPIVTIKIGGQIKEALLDTGADDTVLEDMNLPGKWKPKMIGGIGGFIKVKQYD |
| | Pol: 367-431 | | QWGLTTPDKKHQKDPPFLWMGYELHPDRWTVQPIELPEKESWTVNDIQKLIGKLNWASQIY |
| | AA | | AGIKVAAQEEEEVGFPVRPQVPLRPMTYKGALDLSHFLKEKGGLEGITKLQNFRVYYRDNR |
| | QEE | | DPLWKGPARLLWKGEGAVVIQDNSEIKVVPRRKVKIIRDYGKRMAGDDCVAGRQDEDPKFR |
| | Nef: 64-99 | | LPIQKETWDTWWTDYWQATWIPEWEFTNTPPLVKLWYQLETEPIAGVETFYVDGASNRETK |
| | Pol: 932-1003 | | AAGTVLIGPTPVNIIGRNLLTQLGCTLNFPISPIDTVPVKLKPGMDGPRVKQWPLTEEKIK |
| | Pol: 542-606 | | ALIEICTEMEKEGKISRIGPENPYNTPIFAIKKKDGTKWRKLVDFRELNKKTQDFWEVQLG |
| | AA | | IPHPSGLKKKKSVTVLDIGDAYFSVPLDKEFRKYTAFTVPSTNNETPGVRYQYNVLPMGWK |
| | Pol: 129-320 | | GSPAIFQCSMT |
| 221 | Pol: 56-117 | 501 | FPQITLWQRPLVTIKIGGQLKEALLDTGADDTVLEEMNLPGRWKPKMIGGIGGFIKVRQYD |
| | Pol: 367-431 | | QWGFTTPDKKHQKEPPFLWMGYELHPDKWTVQPIVLPEKDSWTVNDIQKLVGKLNWASQIY |
| | AA | | PGIKVAAQEEEEVGFPVKPQVPLRPMTFKGALDLSHFLREKGGLEGLIITKIQNFRVYYRD |
| | QEE | | SRDPLWKGPAKLLWKGEGAVVIQDNSDIKVVPRRKAKIIRDYGKQMAGDDCVASRQDEDPK |
| | Nef: 64-99 | | FKLPIQKETWETWWTEYWQATWIPEWEFVNTPPLVKLWYQLEKEPIVGAETFYVDGAANRE |
| | Pol: 932-1003 | | TKAAGTVLVGPTPVNIIGRNLLTQIGCTLNFPISPIETVPVKLKPGMDGPKVKQWPLTEEK |
| | Pol: 542-606 | | IKALVEICTEMEKEGKISKIGPENPYNTPVFAIKKKDSTKWRKLVDFRELNKRTQDFWEVQ |
| | AA | | LGIPHPAGLKKKKSVTVLDVGDAYFSVPLDKDFRKYTAFTIPSINNETPGIRYQYNVLPQG |
| | Pa1: 129-320 | | WKGSPAIFQSSMT |
| 98 | Pol: 747-827 | 504 | VAKEIVASCDKCQLKGEAMHGQVDCSPGIWQLDCTHLEGKIILVAVHVASGYIEAEVIPAE |
| | Nef: 117-148 | | TGQETAYFLLKLAGRWPVKTTQGYFPDWQNYTPGPGTRYPLTFGWCFKLVPVTVKAACWWA |
| | Pol: 840-920 | | GIKQEFGIPYNPQSQGVVESMNKELKKIIGQVRDQAEHLKTAVQMAVFIHNFKRKGGIGGY |
| | AA | | SAGERIVDIIAAAMAARASVLSGGELDRWEKIRLRPGGKKKYRLKHIVWASRELERFAVNP |
| | Gag: 1-53 | | GLLETSEGISPRTLNAWVKVVEEKAFSPEVIPMFSALSEGATPQDLNTMLNTVGGHQAAMQ |
| | SEG | | MLKETINEEAAEWDRLHPVHAGPIAPGQMREPRGSDIAGTTSTLQEQIGWMTNNPPIPVGE |
| | Gag: 147-369 | | IYKRWIILGLNKIVRMYSPTSILDIRQGPKEPFRDYVDRFYKTLRAEQASQEVKNWMTETL |
| | AAA | | LVQNANPDCKTILKALGPAATLEEMMTACQGVGGPGHKARVLAEAMSQAAAKEKVYLAWVP |
| | Pol: 683-708 | | AHKGIGGNEQVDKLVS |
| 99 | Gag: 147-369 | 499 | LSPRTLNAWVKVIEEKAFSPEVIPMFTALSEGATPHDLNTMLNTIGGHQAAMQMLKDTINE |
| | Pol: 840-920 | | EAAEWDRVHPVHAGPVAPGQMRDPRGSDIAGSTSTLQEQIAWMTNNPPIPVGDIYKRWIIM |
| | Pol: 683-708 | | GLNKIVRMYSPVSILDIKQGPKEPFRDYVDRFYRTLRAEQASQDVKNWMTETLLVQNSNPD |
| | AAY | | CKTILKALGPGATLEEMMSACQGVGGPSHKARVLAEAMCQAVKAACWWAGVKQEFGIPYNT |
| | Gag: 1-53 | | QSQGVVESMNNELKKIIGQIRDQAEHLKTAVQMAVLIHNFKRKGGIGEYSAGERIIDIIAK |
| | Nef: 117-148 | | EKIYLAWVPAHKGIGGNEQIDKLVSAAYMAARASILSGGKLDKWEKIRLRPGGRKKYKLKH |
| | Pol: 747-827 | | LVWASRELERFALNPGLLETTQGFFPDWQNYTPGPGIRFPLTFGWCFKLVPLVAKEIVACC |
| | | | DKCQLKGEAIHGQVDCSPGVWQLDCTHLEGKVILVAVHVASGYIEAEIIPTETGQETAYFI |
| | | | LKLAGRWPVTT |
| 100 | Pol: 932-1003 | 499 | ITKIQNFRVYYRDSRDPLWKGPAKLLWKGEGAVVIQDNSDIKVVPRRKAKIIRDYGKQMAG |
| | AAY | | DDCVASRQDEDAAYEEEEVGFPVKPQVPLRPMTFKGALDLSHFLREKGGLEGAAWGFTTPD |
| | EE | | KKHQKEPPFLWMGYELHPDKWTVQPIVLPEKDSWTVNDIQKLVGKLNWASQIYPGIKVPKF |
| | Nef: 64-99 | | KLPIQKETWETWWTEYWQATWIPEWEFVNTPPLVKLWYQLEKEPIVGAETFYVDGAANRET |
| | AA | | KFPQITLWQRPLVTIKIGGQLKEALLDTGADDTVLEEMNLPGRWKPKMIGGIGGFIKVRQY |
| | Pol: 367-431 | | DQGTVLVGPTPVNIIGRNLLTQIGCTLNFPISPIETVPVKLKPGMDGPKVKQWPLTEEKIK |
| | Pol: 542-606 | | ALVEICTEMEKEGKISKIGPENPYNTPVFAIKKKDSTKWRKLVDFRELNKRTQDFWEVQLG |
| | Pol: 56-117 | | IPHPAGLKKKKSVTVLDVGDAYFSVPLDKDFRKYTAFTIPSINNETPGIRYQYNVLPQGWK |
| | Pol: 129-320 | | GSPAIFQSSMT |
| 101 | Pol: 542-606 | 493 | PKFRLPIQKETWDTWWTDYWQATWIPEWEFTNTPPLVKLWYQLETEPIAGVETFYVDGASN |
| | Nef: 64-99 | | RETKEEVGFPVRPQVPLRPMTYKGALDLSHFLKEKGGLEGLPQITLWQRPIVTIKIGGQIK |
| | Pol: 56-117 | | EALLDTGADDTVLEDMNLPGKWKPKMIGGIGGFIKVKQYDQITKLQNFRVYYRDNRDPLWK |
| | Pol: 932-1003 | | GPARLLWKGEGAVVIQDNSEIKVVPRRKVKIIRDYGKRMAGDDCVAGRQDEDWGLTTPDKK |
| | Pol: 367-431 | | HQKDPPFLWMGYELHPDRWTVQPIELPEKESWTVNDIQKLIGKLNWASQIYAGIKVKGTVL |
| | K | | IGPTPVNIIGRNLLTQLGCTLNFPISPIDTVPVKLKPGMDGPRVKQWPLTEEKIKALIEIC |
| | Pol: 129-320 | | TEMEKEGKISRIGPENPYNTPIFAIKKKDGTKWRKLVDFRELNKKTQDFWEVQLGIPHPSG |
| | | | LKKKKSVTVLDIGDAYFSVPLDKEFRKYTAFTVPSTNNETPGVRYQYNVLPMGWKGSPAIF |
| | | | QCSMT |

### Compound Fusion Polypeptides

In various embodiments, provided are compound fusion polypeptides comprising two or more fusion polypeptides, as described herein. Such compound fusion polypeptides can be delivered in viral vectors that can express a polypeptide having at least about 750 amino acids in length, e.g., at least about 800, 850, 900, 950, 1000, 1050, 1100 amino acids in length, or longer. As appropriate, the two or more fusion polypeptides can be directly fused, or joined or connected by one or more linkers. In some embodiments, provided are compound fusion polypeptides comprising at least a first fusion polypeptide and a second fusion polypeptide, as described herein, the first and second fusion polypeptides optionally joined or connected by one or more linkers, as described herein, e.g., a cleavable linker such as a 2A cleavable peptide linker.

In various embodiments, the first fusion polypeptide and the second fusion polypeptide in the compound fusion polypeptide comprises the same polypeptide segments, e.g., same amino acid residue position ranges. In some embodiments, the first fusion polypeptide and the second fusion polypeptide in the compound fusion polypeptide are bivalent. For example, in some embodiments, the first fusion polypeptide and the second fusion polypeptide comprise or consist of the following polypeptide segments, wherein the Gag, Pol and Nef amino acid position numbers correspond to SEQ ID NOs: 1, 2 and 3, respectively:
a) amino acid residues corresponding to: Gag 1-53; Gag 147-369; Pol 683-708 and Nef 117-148;
b) amino acid residues corresponding to: Pol 56-117; Pol 129-320; Pol 367-431 and Nef 64-99;
c) amino acid residues corresponding to: Pol 542-606; Pol 747-827; Pol 840-920; Pol 932-1003 and Nef 64-99;
d) amino acid residues corresponding to: Gag 1-53; Gag 147-369; Pol 683-708; Pol 747-827; Pol 840-920 and Nef 117-148;
e) amino acid residues corresponding to: Pol 56-117; Pol 129-320; Pol 367-431; Pol 542-606; Pol 932-1003 and Nef 64-99;
f) amino acid residues corresponding to: Gag 147-369, Pol 586-606, Pol 683-708 and Pol 840-920;
g) amino acid residues corresponding to: Pol 129-320, Pol 747-827, Pol 932-1003 and Nef 64-76; or
h) amino acid residues corresponding to: Gag: 147-369, Pol 747-827, Pol 840-909 and Nef 64-76.

In various embodiments, the first fusion polypeptide and the second fusion polypeptide in the compound fusion polypeptide comprises different polypeptide segments, e.g., same amino acid residue position ranges. In some embodiments, the first fusion polypeptide and the second fusion polypeptide are bivalent. For example, in some embodiments, the compound fusion polypeptide (inclusive of the first fusion polypeptide and the second fusion polypeptide) comprises or consists of the following polypeptide segments, wherein the Gag, Pol and Nef amino acid position numbers correspond to SEQ ID NOs: 1, 2 and 3, respectively:
a) amino acid residues corresponding to: Gag 147-369, Pol 129-320, Pol 586-606, Pol 683-708, Pol 747-827, Pol 840-920, Pol 932-1003 and Nef 64-76; or
b) Gag 1-53, Gag 147-369, Pol 56-117, Pol 129-320, Pol 367-431, Pol 542-606, Pol 683-708, Pol 747-827, Pol 840-920, Pol 932-1003, Nef 64-99 and Nef 117-148.

In some embodiments, the compound fusion polypeptide comprises the following polypeptide segments comprising in sequential order, from N-terminus to C-terminus, optionally joined or connected by one or more linkers:
a) SEQ ID NOs: 6, 4, 16 and 26, and SEQ ID NOs: 7, 5, 27 and 17, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 4, 16 and 26, and SEQ ID NOs: 7, 5, 27 and 17, respectively;
b) SEQ ID NOs: 12, 24, 8 and 10, and SEQ ID NOs: 9, 25, 13 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 12, 24, 8 and 10, and SEQ ID NOs: 9, 25, 13 and 11, respectively;
c) SEQ ID NOs: 14, 22, 18, 24 and 20, and SEQ ID NOs: 15, 25, 19, 23 and 21, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 14, 22, 18, 24 and 20, and SEQ ID NOs: 15, 25, 19, 23 and 21, respectively;
d) SEQ ID NOs: 26, 16, 4 and 6, and SEQ ID NOs: 7, 27, 5 and 17, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 16, 4 and 6, and SEQ ID NOs: 7, 27, 5 and 17, respectively;
e) SEQ ID NOs: 8, 24, 12 and 10, and SEQ ID NOs: 13, 25, 9 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 24, 12 and 10, and SEQ ID NOs: 13, 25, 9 and 11, respectively;
f) SEQ ID NOs: 24, 6, 16 and 18, and SEQ ID NOs: 7, 25, 17 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 16 and 18, and SEQ ID NOs: 7, 25, 17 and 19, respectively;
g) SEQ ID NOs: 26, 10, 20 and 28, and SEQ ID NOs: 21, 27, 11 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 26, 10, 20 and 28, and SEQ ID NOs: 21, 27, 11 and 29, respectively;
h) SEQ ID NOs: 24, 6, 16 and 18, and SEQ ID NOs: 26, 10, 20 and 28, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 16 and 18, and SEQ ID NOs: 26, 10, 20 and 28, respectively;
i) SEQ ID NOs: 7, 25, 17 and 19, and SEQ ID NOs: 21, 27, 11 and 29, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 25, 17 and 19, and SEQ ID NOs: 21, 27, 11 and 29, respectively;
j) SEQ ID NOs: 22, 6, 16, 20, 18, 28, 26 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 6, 16, 20, 18, 28, 26 and 10, respectively;
k) SEQ ID NOs: 7, 21, 19, 17, 27, 25, 29 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 19, 17, 27, 25, 29 and 11, respectively;
l) SEQ ID NOs: 22, 24, 14, 18 and 20, and SEQ ID NOs: 25, 23, 15, 21 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 22, 24, 14, 18 and 20, and SEQ ID NOs: 25, 23, 15, 21 and 19, respectively;
m) SEQ ID NOs: 20, 6, 4, 18, 16 and 26, and SEQ ID NOs: 7, 19, 5, 21, 27 and 17, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 20, 6, 4, 18, 16 and 26, and SEQ ID NOs: 7, 19, 5, 21, 27 and 17, respectively;
n) SEQ ID NOs: 8, 24, 14, 12, 22 and 10, and SEQ ID NOs: 9, 13, 25, 23, 15 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 24, 14, 12, 22 and 10, and SEQ ID NOs: 9, 13, 25, 23, 15 and 11, respectively;
o) SEQ ID NOs: 18, 26, 20, 4, 6 and 16, and SEQ ID NOs: 7, 21, 17, 5, 27 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 18, 26, 20, 4, 6 and 16, SEQ ID NOs: 7, 21, 17, 5, 27 and 19, respectively;
p) SEQ ID NOs: 24, 6, 4, 20, 18, and 32, and SEQ ID NOs: 7, 21, 5, 25, 33 and 19, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 4, 20, 18, and 32, SEQ ID NOs: 7, 21, 5, 25, 33 and 19, respectively;
q) SEQ ID NOs: 6, 20, 4, 24, 32, 18, and SEQ ID NOs: 8, 12, 30, 26, 14 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 4, 24, 32, 18, and SEQ ID NOs: 8, 12, 30, 26, 14 and 10, respectively;
r) SEQ ID NOs: 8, 30, 14, 12, 26, and 10, and SEQ ID NOs: 9, 13, 31, 27, 15 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 8, 30, 14, 12, 26, and 10, SEQ ID NOs: 9, 13, 31, 27, 15 and 11, respectively;
s) SEQ ID NOs: 24, 6, 4, 20, 18 and 32, and SEQ ID NOs: 8, 30, 14, 12, 26 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 24, 6, 4, 20, 18 and 32, and SEQ ID NOs: 8, 30, 14, 12, 26 and 10, respectively;
t) SEQ ID NOs: 6, 20, 4, 24, 32, 18, and SEQ ID NOs: 8, 12, 30, 26, 14 and 10, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 6, 20, 4, 24, 32, 18, and SEQ ID NOs: 8, 12, 30, 26, 14 and 10, respectively; or
u) SEQ ID NOs: 7, 21, 5, 25, 33 and 19, and SEQ ID NOs: 9, 13, 31, 27, 15 and 11, or sequence segments that are at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 7, 21, 5, 25, 33 and 19, and SEQ ID NOs: 9, 13, 31, 27, 15 and 11, respectively.

In various embodiments, the compound fusion polypeptide comprises or consists of the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In various embodiments, the compound fusion polypeptide comprises or consists of the following first fusion polypeptide and second fusion polypeptide in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 75 and 74, or fusion polypeptides that are at least 80%, 81%, 82%, 97%, 98% or 99% identical to any one of SEQ ID NOs: 75 and 74, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively;
SEQ ID NOs: 86 and 87, or fusion polypeptides that are at least 80%, 81%, 82%, 97%, 98% or 99% identical to any one of SEQ ID NOs: 86 and 87, respectively;
SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 87 and 83, or fusion polypeptides that are at least 80%, 81%, 82%, 97%, 98% or 99% identical to any one of SEQ ID NOs: 87 and 83, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively;
SEQ ID NOs: 90 and 91, or fusion polypeptides that are at least 80%, 81%, 82%, 97%, 98% or 99% identical to any one of SEQ ID NOs: 90 and 91, respectively;
SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 97 and 95, or fusion polypeptides that are at least 80%, 81%, 82%, 97%, 98% or 99% identical to any one of SEQ ID NOs: 97 and 95, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively;
SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively;
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or
SEQ ID NOs: 101 and 100, or fusion polypeptides that are at least 80%, 81%, 82%, 97%, 98% or 99% identical to any one of SEQ ID NOs: 101 and 100, respectively.

In some embodiments of the compound fusion polypeptide, the first fusion polypeptide and the second fusion polypeptide are joined or connected by a cleavable linker. In various embodiments, the first fusion polypeptide and the second fusion polypeptide are joined or connected by a cleavable linker selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. RAKR (SEQ ID NO: 60), REKR (SEQ ID NO: 61), RRKR (SEQ ID NO: 62)), and combinations, derivatives or variants thereof. In some embodiments, the first fusion polypeptide and the second fusion polypeptide are joined or connected by a furin recognition/cleavage site selected from RAKR (SEQ ID NO: 60), REKR (SEQ ID NO: 61) and RRKR (SEQ ID NO: 62). In some embodiments, the first fusion polypeptide and the second fusion polypeptide are joined or connected by a 2A cleavable peptide comprising or consisting of the amino acid sequence of ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67), or having an amino acid sequence that is at least 90%, 91%, 92%, 93%, 94%, 95%, 96%, 97%, 98% or at least 99% identical to ATNFSLLKQAGDVEENPGP (SEQ ID NO: 63), APVKQTLNFDLLKLAGDVESNPGP (SEQ ID NO: 64), RAKRAPVKQTLNFDLLKLAGDVESNPGP (SEQ ID NO: 65), QCTNYALLKLAGDVESNPGP (SEQ ID NO: 66), or EGRGSLLTCGDVEENPGP (SEQ ID NO: 67).

Illustrative compound fusion polypeptides, without signal sequences, which have been designed and assembled according to the herein described methods, are provided in Table F. Table F discloses “AAA” as SEQ ID NO: 48, “LIK” as SEQ ID NO: 53, “AAY” as SEQ ID NO: 49, “SEG” as SEQ ID NO: 55, “QEE” as SEQ ID NO: 51, “RAKR” as SEQ ID NO: 60 and “PPV” as SEQ ID NO: 54.

In some embodiments, the compound fusion polypeptide comprises or consists of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the compound fusion polypeptide comprises or consists of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the compound fusion polypeptide comprises or consists of an amino acid sequence of SEQ ID NO: 209, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 209. In some embodiments, the compound fusion polypeptide comprises or consists of an amino acid sequence of SEQ ID NO: 222, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 222. In some embodiments, the compound fusion polypeptide comprises or consists of an amino acid sequence of SEQ ID NO: 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 223. In some embodiments, the compound fusion polypeptide comprises or consists of an amino acid sequence of SEQ ID NO: 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOs: 227. As appropriate or desired, the compound fusion polypeptide can have an N-terminal methionine residue.

TABLE F
Illustrative Compound Fusion Polypeptides

| SEQ ID | Polypeptide | Length | |
|---|---|---|---|
| NO: | segments | (aa) | Amino Acid Sequence |
| 105 | Pol: 840-920 | 1028 | TVKAACWWAGIKQEFGIPYNPQSQGVVESMNKELKKIIGQVRDQAEHLKTAVQMAVFIH |
| | Gag: 147-369 | | NFKRKGGIGGYSAGERIVDIIAISPRTLNAWVKVVEEKAFSPEVIPMFSALSEGATPQD |
| | Gag: 1-53 | | LNTMLNTVGGHQAAMQMLKETINEEAAEWDRLHPVHAGPIAPGQMREPRGSDIAGTTST |
| | PPV | | LQEQIGWMTNNPPIPVGEIYKRWIILGLNKIVRMYSPTSILDIRQGPKEPFRDYVDRFY |
| | Pol: 747-827 | | KTLRAEQASQEVKNWMTETLLVQNANPDCKTILKALGPAATLEEMMTACQGVGGPGHKA |
| | Pol: 683-708 | | RVLAEAMSQMAARASVLSGGELDRWEKIRLRPGGKKKYRLKHIVWASRELERFAVNPGL |
| | Nef: 117-148 | | LETPPVVAKEIVASCDKCQLKGEAMHGQVDCSPGIWQLDCTHLEGKIILVAVHVASGYI |
| | RAKR | | EAEVIPAETGQETAYFLLKLAGRWPVKTKEKVYLAWVPAHKGIGGNEQVDKLVSTQGYF |
| | F2A linker | | PDWQNYTPGPGTRYPLTFGWCFKLVPVRAKRAPVKQTLNFDLLKLAGDVESNPGPLSPR |
| | Gag: 147-369 | | TLNAWVKVIEEKAFSPEVIPMFTALSEGATPHDLNTMLNTIGGHQAAMQMLKDTINEEA |
| | Pol: 747-827 | | AEWDRVHPVHAGPVAPGQMRDPRGSDIAGSTSTLQEQIAWMTNNPPIPVGDIYKRWIIM |
| | Gag: 1-53 | | GLNKIVRMYSPVSILDIKQGPKEPFRDYVDRFYRTLRAEQASQDVKNWMTETLLVQNSN |
| | AA | | PDCKTILKALGPGATLEEMMSACQGVGGPSHKARVLAEAMCQVAKEIVACCDKCQLKGE |
| | Pol: 840-920 | | AIHGQVDCSPGVWQLDCTHLEGKVILVAVHVASGYIEAEIIPTETGQETAYFILKLAGR |
| | Nef: 117-148 | | WPVTTMAARASILSGGKLDKWEKIRLRPGGRKKYKLKHLVWASRELERFALNPGLLETA |
| | LIK | | AAVKAACWWAGVKQEFGIPYNTQSQGVVESMNNELKKIIGQIRDQAEHLKTAVQMAVLI |
| | Pol: 683-708 | | HNFKRKGGIGEYSAGERIIDIIATQGFFPDWQNYTPGPGIRFPLTFGWCFKLVPLLIKK |
| | | | EKIYLAWVPAHKGIGGNEQIDKLVS |
| 206 | M | 1030 | MTVKAACWWAGIKQEFGIPYNPQSQGVVESMNKELKKIIGQVRDQAEHLKTAVQMAVFI |
| | Pol: 840-920 | | HNFKRKGGIGGYSAGERIVDIIAISPRTLNAWVKVVEEKAFSPEVIPMFSALSEGATPQ |
| | Gag: 147-369 | | DLNTMLNTVGGHQAAMQMLKETINEEAAEWDRLHPVHAGPIAPGQMREPRGSDIAGTTS |
| | Gag: 1-53 | | TLQEQIGWMTNNPPIPVGEIYKRWIILGLNKIVRMYSPTSILDIRQGPKEPFRDYVDRF |
| | PPV | | YKTLRAEQASQEVKNWMTETLLVQNANPDCKTILKALGPAATLEEMMTACQGVGGPGHK |
| | Pol: 747-827 | | ARVLAEAMSQMAARASVLSGGELDRWEKIRLRPGGKKKYRLKHIVWASRELERFAVNPG |
| | Pol: 683-708 | | LLETPPVVAKEIVASCDKCQLKGEAMHGQVDCSPGIWQLDCTHLEGKIILVAVHVASGY |
| | Nef: 117-148 | | IEAEVIPAETGQETAYFLLKLAGRWPVKTKEKVYLAWVPAHKGIGGNEQVDKLVSTQGY |
| | RAKR | | FPDWQNYTPGPGTRYPLTFGWCFKLVPVRAKRAPVKQTLNFDLLKLAGDVESNPGPMLS |
| | F2A linker | | PRTLNAWVKVIEEKAFSPEVIPMFTALSEGATPHDLNTMLNTIGGHQAAMQMLKDTINE |
| | M | | EAAEWDRVHPVHAGPVAPGQMRDPRGSDIAGSTSTLQEQIAWMTNNPPIPVGDIYKRWI |
| | Gag: 147-369 | | IMGLNKIVRMYSPVSILDIKQGPKEPFRDYVDRFYRTLRAEQASQDVKNWMTETLLVQN |
| | Pol: 747-827 | | SNPDCKTILKALGPGATLEEMMSACQGVGGPSHKARVLAEAMCQVAKEIVACCDKCQLK |
| | Gag: 1-53 | | GEAIHGQVDCSPGVWQLDCTHLEGKVILVAVHVASGYIEAEIIPTETGQETAYFILKLA |
| | AA | | GRWPVTTMAARASILSGGKLDKWEKIRLRPGGRKKYKLKHLVWASRELERFALNPGLLE |
| | Pol: 840-920 | | TAAAVKAACWWAGVKQEFGIPYNTQSQGVVESMNNELKKIIGQIRDQAEHLKTAVQMAV |
| | Nef: 117-148 | | LIHNFKRKGGIGEYSAGERIIDIIATQGFFPDWQNYTPGPGIRFPLTFGWCFKLVPLLI |
| | LIK | | KKEKIYLAWVPAHKGIGGNEQIDKLVS |
| | Pol: 683-708 | | |
| 106 | Gag: 147-369 | 1028 | LSPRTLNAWVKVIEEKAFSPEVIPMFTALSEGATPHDLNTMLNTIGGHQAAMQMLKDTI |
| | Pol: 747-827 | | NEEAAEWDRVHPVHAGPVAPGQMRDPRGSDIAGSTSTLQEQIAWMTNNPPIPVGDIYKR |
| | Gag: 1-53 | | WIIMGLNKIVRMYSPVSILDIKQGPKEPFRDYVDRFYRTLRAEQASQDVKNWMTETLLV |
| | AA | | QNSNPDCKTILKALGPGATLEEMMSACQGVGGPSHKARVLAEAMCQVAKEIVACCDKCQ |
| | Pol: 840-920 | | LKGEAIHGQVDCSPGVWQLDCTHLEGKVILVAVHVASGYIEAEIIPTETGQETAYFILK |
| | Nef: 117-148 | | LAGRWPVTTMAARASILSGGKLDKWEKIRLRPGGRKKYKLKHLVWASRELERFALNPGL |
| | LIK | | LETAAAVKAACWWAGVKQEFGIPYNTQSQGVVESMNNELKKIIGQIRDQAEHLKTAVQM |
| | Pol: 683-708 | | AVLIHNFKRKGGIGEYSAGERIIDIIATQGFFPDWQNYTPGPGIRFPLTFGWCFKLVPL |
| | RAKR | | LIKKEKIYLAWVPAHKGIGGNEQIDKLVSRAKRAPVKQTLNFDLLKLAGDVESNPGPTV |
| | F2A linker | | KAACWWAGIKQEFGIPYNPQSQGVVESMNKELKKIIGQVRDQAEHLKTAVQMAVFIHNF |
| | Pol: 840-920 | | KRKGGIGGYSAGERIVDIIAISPRTLNAWVKVVEEKAFSPEVIPMFSALSEGATPQDLN |
| | Gag: 147-369 | | TMLNTVGGHQAAMQMLKETINEEAAEWDRLHPVHAGPIAPGQMREPRGSDIAGTTSTLQ |
| | Gag: 1-53 | | EQIGWMTNNPPIPVGEIYKRWIILGLNKIVRMYSPTSILDIRQGPKEPFRDYVDRFYKT |
| | PPV | | LRAEQASQEVKNWMTETLLVQNANPDCKTILKALGPAATLEEMMTACQGVGGPGHKARV |
| | Pol: 747-827 | | LAEAMSQMAARASVLSGGELDRWEKIRLRPGGKKKYRLKHIVWASRELERFAVNPGLLE |
| | Pol: 683-708 | | TPPVVAKEIVASCDKCQLKGEAMHGQVDCSPGIWQLDCTHLEGKIILVAVHVASGYIEA |
| | Nef: 117-148 | | EVIPAETGQETAYFLLKLAGRWPVKTKEKVYLAWVPAHKGIGGNEQVDKLVSTQGYFPD |
| | | | WQNYTPGPGTRYPLTFGWCFKLVPV |
| 107 | Pol: 56-117 | 1022 | FPQITLWQRPLVTIKIGGQLKEALLDTGADDTVLEEMNLPGRWKPKMIGGIGGFIKVRQ |
| | Nef: 64-99 | | YDQEEVGFPVKPQVPLRPMTFKGALDLSHFLREKGGLEGLIPKFKLPIQKETWETWWTE |
| | LI | | YWQATWIPEWEFVNTPPLVKLWYQLEKEPIVGAETFYVDGAANRETKWGFTTPDKKHQK |
| | Pol: 542-606 | | EPPFLWMGYELHPDKWTVQPIVLPEKDSWTVNDIQKLVGKLNWASQTYPGIKVITKIQN |
| | Pol: 367-431 | | FRVYYRDSRDPLWKGPAKLLWKGEGAVVIQDNSDIKVVPRRKAKIIRDYGKQMAGDDCV |
| | Pol: 932-1003 | | ASRQDEDGTVLVGPTPVNIIGRNLLTQIGCTLNFPISPIETVPVKLKPGMDGPKVKQWP |
| | Pol: 129-320 | | LTEEKIKALVEICTEMEKEGKISKIGPENPYNTPVFAIKKKDSTKWRKLVDFRELNKRT |
| | RAKR | | QDFWEVQLGIPHPAGLKKKKSVTVLDVGDAYFSVPLDKDFRKYTAFTIPSINNETPGIR |
| | F2A linker | | YQYNVLPQGWKGSPAIFQSSMTRAKRAPVKQTLNFDLLKLAGDVESNPGPNLPQITLWQ |
| | Pol: 56-117 | | RPIVTIKIGGQIKEALLDTGADDTVLEDMNLPGKWKPKMIGGIGGFIKVKQYDQWGLTT |
| | Pol: 367-431 | | PDKKHQKDPPFLWMGYELHPDRWTVQPIELPEKESWTVNDIQKLIGKLNWASQIYAGIK |
| | AA | | VAAQEEEEVGFPVRPQVPLRPMTYKGALDLSHFLKEKGGLEGITKLQNFRVYYRDNRDP |
| | QEE | | LWKGPARLLWKGEGAVVIQDNSEIKVVPRRKVKIIRDYGKRMAGDDCVAGRQDEDPKFR |
| | Nef: 64-99 | | LPIQKETWDTWWTDYWQATWIPEWEFTNTPPLVKLWYQLETEPIAGVETFYVDGASNRE |
| | Pol: 932-1003 | | TKAAGTVLIGPTPVNIIGRNLLTQLGCTLNFPISPIDTVPVKLKPGMDGPRVKQWPLTE |
| | Pol: 542-606 | | EKIKALIEICTEMEKEGKISRIGPENPYNTPIFAIKKKDGTKWRKLVDFRELNKKTQDF |
| | AA | | WEVQLGIPHPSGLKKKKSVTVLDIGDAYFSVPLDKEFRKYTAFTVPSTNNETPGVRYQY |
| | Pol: 129-320 | | NVLPMGWKGSPAIFQCSMT |
| 207 | M | 1023 | MFPQITLWQRPLVTIKIGGQLKEALLDTGADDTVLEEMNLPGRWKPKMIGGIGGFIKVR |
| | Pol: 56-117 | | QYDQEEVGFPVKPQVPLRPMTFKGALDLSHFLREKGGLEGLIPKFKLPIQKETWETWWT |
| | Nef: 64-99 | | EYWQATWIPEWEFVNTPPLVKLWYQLEKEPIVGAETFYVDGAANRETKWGFTTPDKKHQ |
| | LI | | KEPPFLWMGYELHPDKWTVQPIVLPEKDSWTVNDIQKLVGKLNWASQIYPGIKVITKIQ |
| | Pol: 542-606 | | NFRVYYRDSRDPLWKGPAKLLWKGEGAVVIQDNSDIKVVPRRKAKIIRDYGKQMAGDDC |
| | Pol: 367-431 | | VASRQDEDGTVLVGPTPVNIIGRNLLTQIGCTLNFPISPIETVPVKLKPGMDGPKVKQW |
| | Pol: 932-1003 | | PLTEEKIKALVEICTEMEKEGKISKIGPENPYNTPVFAIKKKDSTKWRKLVDFRELNKR |
| | Pol: 129-320 | | TQDFWEVQLGIPHPAGLKKKKSVTVLDVGDAYFSVPLDKDFRKYTAFTIPSINNETPGI |
| | RAKR | | RYQYNVLPQGWKGSPAIFQSSMTRAKRAPVKQTLNFDLLKLAGDVESNPGPMLPQITLW |
| | F2A linker | | QRPIVTIKIGGQIKEALLDTGADDTVLEDMNLPGKWKPKMIGGIGGFIKVKQYDQWGLT |
| | M | | TPDKKHQKDPPFLWMGYELHPDRWTVQPIELPEKESWTVNDIQKLIGKLNWASQIYAGI |
| | Pol: 56-117 | | KVAAQEEEEVGFPVRPQVPLRPMTYKGALDLSHFLKEKGGLEGITKLQNFRVYYRDNRD |
| | Pol: 367-431 | | PLWKGPARLLWKGEGAVVIQDNSEIKVVPRRKVKIIRDYGKRMAGDDCVAGRQDEDPKF |
| | AA | | RLPIQKETWDTWWTDYWQATWIPEWEFTNTPPLVKLWYQLETEPIAGVETFYVDGASNR |
| | QEE | | ETKAAGTVLIGPTPVNIIGRNLLTQLGCTLNFPISPIDTVPVKLKPGMDGPRVKQWPLT |
| | Nef: 64-99 | | EEKIKALIEICTEMEKEGKISRIGPENPYNTPIFAIKKKDGTKWRKLVDFRELNKKTQD |
| | Pol: 932-1003 | | FWEVQLGIPHPSGLKKKKSVTVLDIGDAYFSVPLDKEFRKYTAFTVPSTNNETPGVRYQ |
| | Pol: 542-606 | | YNVLPMGWKGSPAIFQCSMT |
| | AA | | |
| | Pol: 129-320 | | |
| 108 | Pol: 56-117 | 1022 | LPQITLWQRPIVTIKIGGQIKEALLDTGADDTVLEDMNLPGKWKPKMIGGIGGFIKVKQ |
| | Pol: 367-431 | | YDQWGLTTPDKKHQKDPPFLWMGYELHPDRWTVQPIELPEKESWTVNDIQKLIGKLNWA |
| | AA | | SQIYAGIKVAAQEEEEVGFPVRPQVPLRPMTYKGALDLSHFLKEKGGLEGITKLQNFRV |
| | QEE | | YYRDNRDPLWKGPARLLWKGEGAVVIQDNSEIKVVPRRKVKIIRDYGKRMAGDDCVAGR |
| | Nef: 64-99 | | QDEDPKFRLPIQKETWDTWWTDYWQATWIPEWEFTNTPPLVKLWYQLETEPIAGVETFY |
| | Pol: 932-1003 | | VDGASNRETKAAGTVLIGPTPVNIIGRNLLTQLGCTLNFPISPIDTVPVKLKPGMDGPR |
| | Pol: 542-606 | | VKQWPLTEEKIKALIEICTEMEKEGKISRIGPENPYNTPIFAIKKKDGTKWRKLVDFRE |
| | AA | | LNKKTQDFWEVQLGIPHPSGLKKKKSVTVLDIGDAYFSVPLDKEFRKYTAFTVPSTNNE |
| | Pol: 129-320 | | TPGVRYQYNVLPMGWKGSPAIFQCSMTRAKRAPVKQTLNFDLLKLAGDVESNPGPFPQI |
| | RAKR | | TLWQRPLVTIKIGGQLKEALLDTGADDTVLEEMNLPGRWKPKMIGGIGGFIKVRQYDQE |
| | F2A linker | | EVGFPVKPQVPLRPMTFKGALDLSHFLREKGGLEGLIPKFKLPIQKETWETWWTEYWQA |
| | Pol: 56-117 | | TWIPEWEFVNTPPLVKLWYQLEKEPIVGAETFYVDGAANRETKWGFTTPDKKHQKEPPF |
| | Nef: 64-99 | | LWMGYELHPDKWTVQPIVLPEKDSWTVNDIQKLVGKLNWASQIYPGIKVITKIQNFRVY |
| | LI | | YRDSRDPLWKGPAKLLWKGEGAVVIQDNSDIKVVPRRKAKIIRDYGKQMAGDDCVASRQ |
| | Pol: 542-606 | | DEDGTVLVGPTPVNIIGRNLLTQIGCTLNFPISPIETVPVKLKPGMDGPKVKQWPLTEE |
| | Pol: 367-431 | | KIKALVEICTEMEKEGKISKIGPENPYNTPVFAIKKKDSTKWRKLVDFRELNKRTQDFW |
| | Pol: 932-1003 | | EVQLGIPHPAGLKKKKSVTVLDVGDAYFSVPLDKDFRKYTAFTIPSINNETPGIRYQYN |
| | Pol: 129-320 | | VLPQGWKGSPAIFQSSMT |
| 109 | Pol: 747-827 | 1034 | VAKEIVASCDKCQLKGEAMHGQVDCSPGIWQLDCTHLEGKIILVAVHVASGYIEAEVIP |
| | Nef: 117-148 | | AETGQETAYFLLKLAGRWPVKTTQGYFPDWQNYTPGPGTRYPLTFGWCFKLVPVTVKAA |
| | Pol: 840-920 | | CWWAGIKQEFGIPYNPQSQGVVESMNKELKKIIGQVRDQAEHLKTAVQMAVFIHNFKRK |
| | AA | | GGIGGYSAGERIVDIIAAAMAARASVLSGGELDRWEKIRLRPGGKKKYRLKHIVWASRE |
| | Gag: 1-53 | | LERFAVNPGLLETSEGISPRTLNAWVKVVEEKAFSPEVIPMFSALSEGATPQDLNTMLN |
| | SEG | | TVGGHQAAMQMLKETINEEAAEWDRLHPVHAGPIAPGQMREPRGSDIAGTTSTLQEQIG |
| | Gag: 147-369 | | WMTNNPPIPVGEIYKRWIILGLNKIVRMYSPTSILDIRQGPKEPFRDYVDRFYKTLRAE |
| | AAA | | QASQEVKNWMTETLLVQNANPDCKTILKALGPAATLEEMMTACQGVGGPGHKARVLAEA |
| | Pol: 683-708 | | MSQAAAKEKVYLAWVPAHKGIGGNEQVDKLVSRAKRAPVKQTLNFDLLKLAGDVESNPG |
| | RAKR | | PHQALSPRTLNAWVKVIEEKAFSPEVIPMFTALSEGATPHDLNTMLNTIGGHQAAMQML |
| | F2A linker | | KDTINEEAAEWDRVHPVHAGPVAPGQMRDPRGSDIAGSTSTLQEQIAWMTNNPPIPVGD |
| | Gag: 147-369 | | IYKRWIIMGLNKIVRMYSPVSILDIKQGPKEPFRDYVDRFYRTLRAEQASQDVKNWMTE |
| | Pol: 840-920 | | TLLVQNSNPDCKTILKALGPGATLEEMMSACQGVGGPSHKARVLAEAMCQAVKAACWWA |
| | Pol: 683-708 | | GVKQEFGIPYNTQSQGVVESMNNELKKIIGQIRDQAEHLKTAVQMAVLIHNFKRKGGIG |
| | AAY | | EYSAGERIIDIIAKEKIYLAWVPAHKGIGGNEQIDKLVSAAYMAARASILSGGKLDKWE |
| | Gag: 1-53 | | KIRLRPGGRKKYKLKHLVWASRELERFALNPGLLETTQGFFPDWQNYTPGPGIRFPLTF |
| | Nef: 117-148 | | GWCFKLVPLVAKEIVACCDKCQLKGEAIHGQVDCSPGVWQLDCTHLEGKVILVAVHVAS |
| | Pol: 747-827 | | GYIEAEIIPTETGQETAYFILKLAGRWPVTT |
| 110 | Gag: 147-369 | 1031 | LSPRTLNAWVKVIEEKAFSPEVIPMFTALSEGATPHDLNTMLNTIGGHQAAMQMLKDTI |
| | Pol: 840-920 | | NEEAAEWDRVHPVHAGPVAPGQMRDPRGSDIAGSTSTLQEQIAWMTNNPPIPVGDIYKR |
| | Pol: 683-708 | | WIIMGLNKIVRMYSPVSILDIKQGPKEPFRDYVDRFYRTLRAEQASQDVKNWMTETLLV |
| | AAY | | QNSNPDCKTILKALGPGATLEEMMSACQGVGGPSHKARVLAEAMCQAVKAACWWAGVKQ |
| | Gag: 1-53 | | EFGIPYNTQSQGVVESMNNELKKIIGQIRDQAEHLKTAVQMAVLIHNFKRKGGIGEYSA |
| | Nef: 117-148 | | GERIIDIIAKEKIYLAWVPAHKGIGGNEQIDKLVSAAYMAARASILSGGKLDKWEKIRL |
| | Pol: 747-827 | | RPGGRKKYKLKHLVWASRELERFALNPGLLETTQGFFPDWQNYTPGPGIRFPLTFGWCF |
| | RAKR | | KLVPLVAKEIVACCDKCQLKGEAIHGQVDCSPGVWQLDCTHLEGKVILVAVHVASGYIE |
| | F2A linker | | AEIIPTETGQETAYFILKLAGRWPVTTRAKRAPVKQTLNFDLLKLAGDVESNPGPVAKE |
| | Pol: 747-827 | | IVASCDKCQLKGEAMHGQVDCSPGIWQLDCTHLEGKIILVAVHVASGYIEAEVIPAETG |
| | Nef: 117-148 | | QETAYFLLKLAGRWPVKTTQGYFPDWQNYTPGPGTRYPLTFGWCFKLVPVTVKAACWWA |
| | Pol: 840-920 | | GIKQEFGIPYNPQSQGVVESMNKELKKIIGQVRDQAEHLKTAVQMAVFIHNFKRKGGIG |
| | AA | | GYSAGERIVDIIAAAMAARASVLSGGELDRWEKIRLRPGGKKKYRLKHIVWASRELERF |
| | Gag: 1-53 | | AVNPGLLETSEGISPRTLNAWVKVVEEKAFSPEVIPMFSALSEGATPQDLNTMLNTVGG |
| | SEG | | HQAAMQMLKETINEEAAEWDRLHPVHAGPIAPGQMREPRGSDIAGTTSTLQEQIGWMTN |
| | Gag: 147-369 | | NPPIPVGEIYKRWIILGLNKIVRMYSPTSILDIRQGPKEPFRDYVDRFYKTLRAEQASQ |
| | AAA | | EVKNWMTETLLVQNANPDCKTILKALGPAATLEEMMTACQGVGGPGHKARVLAEAMSQA |
| | Pol: 683-708 | | AAKEKVYLAWVPAHKGIGGNEQVDKLVS |
| 111 | Pol: 932-1003 | 1020 | ITKIQNFRVYYRDSRDPLWKGPAKLLWKGEGAVVIQDNSDIKVVPRRKAKIIRDYGKQM |
| | AAY | | AGDDCVASRQDEDAAYEEEEVGFPVKPQVPLRPMTFKGALDLSHFLREKGGLEGAAWGF |
| | EE | | TTPDKKHQKEPPFLWMGYELHPDKWTVQPIVLPEKDSWTVNDIQKLVGKLNWASQIYPG |
| | Nef: 64-99 | | IKVPKFKLPIQKETWETWWTEYWQATWIPEWEFVNTPPLVKLWYQLEKEPIVGAETFYV |
| | AA | | DGAANRETKFPQITLWQRPLVTIKIGGQLKEALLDTGADDTVLEEMNLPGRWKPKMIGG |
| | Pol: 367-431 | | IGGFIKVRQYDQGTVLVGPTPVNIIGRNLLTQIGCTLNFPISPIETVPVKLKPGMDGPK |
| | Pol: 542-606 | | VKQWPLTEEKIKALVEICTEMEKEGKISKIGPENPYNTPVFAIKKKDSTKWRKLVDFRE |
| | Pol: 56-117 | | LNKRTQDFWEVQLGIPHPAGLKKKKSVTVLDVGDAYFSVPLDKDFRKYTAFTIPSINNE |
| | Pol: 129-320 | | TPGIRYQYNVLPQGWKGSPAIFQSSMTRAKRAPVKQTLNFDLLKLAGDVESNPGPPKFR |
| | RAKR | | LPIQKETWDTWWTDYWQATWIPEWEFTNTPPLVKLWYQLETEPIAGVETFYVDGASNRE |
| | F2A linker | | TKEEVGFPVRPQVPLRPMTYKGALDLSHFLKEKGGLEGLPQITLWQRPIVTIKIGGQIK |
| | Pol: 542-606 | | EALLDTGADDTVLEDMNLPGKWKPKMIGGIGGFIKVKQYDQITKLQNFRVYYRDNRDPL |
| | Nef: 64-99 | | WKGPARLLWKGEGAVVIQDNSEIKVVPRRKVKIIRDYGKRMAGDDCVAGRQDEDWGLTT |
| | Pol: 56-117 | | PDKKHQKDPPFLWMGYELHPDRWTVQPIELPEKESWTVNDIQKLIGKLNWASQIYAGIK |
| | Pol: 932-1003 | | VKGTVLIGPTPVNIIGRNLLTQLGCTLNFPISPIDTVPVKLKPGMDGPRVKQWPLTEEK |
| | Pol: 367-431 | | IKALIEICTEMEKEGKISRIGPENPYNTPIFAIKKKDGTKWRKLVDFRELNKKTQDFWE |
| | K | | VQLGIPHPSGLKKKKSVTVLDIGDAYFSVPLDKEFRKYTAFTVPSTNNETPGVRYQYNV |
| | Pol: 129-320 | | LPMGWKGSPAIFQCSMT |
| 112 | Pol: 542-606 | 1020 | PKFRLPIQKETWDTWWTDYWQATWIPEWEFTNTPPLVKLWYQLETEPIAGVETFYVDGA |
| | Nef: 64-99 | | SNRETKEEVGFPVRPQVPLRPMTYKGALDLSHFLKEKGGLEGLPQITLWQRPIVTIKIG |
| | Pol: 56-117 | | GQIKEALLDTGADDTVLEDMNLPGKWKPKMIGGIGGFIKVKQYDQITKLQNFRVYYRDN |
| | Pol: 932-1003 | | RDPLWKGPARLLWKGEGAVVIQDNSEIKVVPRRKVKIIRDYGKRMAGDDCVAGRQDEDW |
| | Pol: 367-431 | | GLTTPDKKHQKDPPFLWMGYELHPDRWTVQPIELPEKESWTVNDIQKLIGKLNWASQIY |
| | K | | AGIKVKGTVLIGPTPVNIIGRNLLTQLGCTLNFPISPIDTVPVKLKPGMDGPRVKQWPL |
| | Pol: 129-320 | | TEEKIKALIEICTEMEKEGKISRIGPENPYNTPIFAIKKKDGTKWRKLVDFRELNKKTQ |
| | RAKR | | DFWEVQLGIPHPSGLKKKKSVTVLDIGDAYFSVPLDKEFRKYTAFTVPSTNNETPGVRY |
| | F2A linker | | QYNVLPMGWKGSPAIFQCSMTRAKRAPVKQTLNFDLLKLAGDVESNPGPITKIQNFRVY |
| | Pol: 932-1003 | | YRDSRDPLWKGPAKLLWKGEGAVVIQDNSDIKVVPRRKAKIIRDYGKQMAGDDCVASRQ |
| | AAY | | DEDAAYEEEEVGFPVKPQVPLRPMTFKGALDLSHFLREKGGLEGAAWGFTTPDKKHQKE |
| | EE | | PPFLWMGYELHPDKWTVQPIVLPEKDSWTVNDIQKLVGKLNWASQIYPGIKVPKFKLPI |
| | Nef: 64-99 | | QKETWETWWTEYWQATWIPEWEFVNTPPLVKLWYQLEKEPIVGAETFYVDGAANRETKF |
| | AA | | PQITLWQRPLVTIKIGGQLKEALLDTGADDTVLEEMNLPGRWKPKMIGGIGGFIKVRQY |
| | Pol: 367-431 | | DQGTVLVGPTPVNIIGRNLLTQIGCTLNFPISPIETVPVKLKPGMDGPKVKQWPLTEEK |
| | Pol: 542-606 | | IKALVEICTEMEKEGKISKIGPENPYNTPVFAIKKKDSTKWRKLVDFRELNKRTQDFWE |
| | Pol: 56-117 | | VQLGIPHPAGLKKKKSVTVLDVGDAYFSVPLDKDFRKYTAFTIPSINNETPGIRYQYNV |
| | Pol: 129-320 | | LPQGWKGSPAIFQSSMT |
| 208 | M | 1023 | MTVKAACWWAGIKQEFGIPYNPQSQGVVESMNKELKKIIGQVRDQAEHLKTAVQMAVFI |
| | Pol: 840-920 | | HNFKRKGGIGGYSAGERIVDIIAISPRTLNAWVKVVEEKAFSPEVIPMFSALSEGATPQ |
| | Gag: 147-369 | | DLNTMLNTVGGHQAAMQMLKETINEEAAEWDRLHPVHAGPIAPGQMREPRGSDIAGTTS |
| | Gag: 1-53 | | TLQEQIGWMTNNPPIPVGEIYKRWIILGLNKIVRMYSPTSILDIRQGPKEPFRDYVDRF |
| | PPV | | YKTLRAEQASQEVKNWMTETLLVQNANPDCKTILKALGPAATLEEMMTACQGVGGPGHK |
| | Pol: 747-827 | | ARVLAEAMSQMAARASVLSGGELDRWEKIRLRPGGKKKYRLKHIVWASRELERFAVNPG |
| | Pol: 683-708 | | LLETPPVVAKEIVASCDKCQLKGEAMHGQVDCSPGIWQLDCTHLEGKIILVAVHVASGY |
| | Nef: 117-148 | | IEAEVIPAETGQETAYFLLKLAGRWPVKTKEKVYLAWVPAHKGIGGNEQVDKLVSTQGY |
| | RAKR | | FPDWQNYTPGPGTRYPLTFGWCFKLVPVRAKRAPVKQTLNFDLLKLAGDVESNPGPMFP |
| | F2A linker | | QITLWQRPLVTIKIGGQLKEALLDTGADDTVLEEMNLPGRWKPKMIGGIGGFIKVRQYD |
| | M | | QEEVGFPVKPQVPLRPMTFKGALDLSHFLREKGGLEGLIPKFKLPIQKETWETWWTEYW |
| | Pol: 56-117 | | QATWIPEWEFVNTPPLVKLWYQLEKEPIVGAETFYVDGAANRETKWGFTTPDKKHQKEP |
| | Nef: 64-99 | | PFLWMGYELHPDKWTVQPIVLPEKDSWTVNDIQKLVGKLNWASQIYPGIKVITKIQNFR |
| | LI | | VYYRDSRDPLWKGPAKLLWKGEGAVVIQDNSDIKVVPRRKAKIIRDYGKQMAGDDCVAS |
| | Pol: 542-606 | | RQDEDGTVLVGPTPVNIIGRNLLTQIGCTLNFPISPIETVPVKLKPGMDGPKVKQWPLT |
| | Pol: 367-431 | | EEKIKALVEICTEMEKEGKISKIGPENPYNTPVFAIKKKDSTKWRKLVDFRELNKRTQD |
| | Pol: 932-1003 | | FWEVQLGIPHPAGLKKKKSVTVLDVGDAYFSVPLDKDFRKYTAFTIPSINNETPGIRYQ |
| | Pol: 129-320 | | YNVLPQGWKGSPAIFQSSMT |
| 224 | Pol: 840-920 | 1021 | TVKAACWWAGIKQEFGIPYNPQSQGVVESMNKELKKIIGQVRDQAEHLKTAVQMAVFIH |
| | Gag: 147-369 | | NFKRKGGIGGYSAGERIVDIIAISPRTLNAWVKVVEEKAFSPEVIPMFSALSEGATPQD |
| | Gag: 1-53 | | LNTMLNTVGGHQAAMQMLKETINEEAAEWDRLHPVHAGPIAPGQMREPRGSDIAGTTST |
| | PPV | | LQEQIGWMTNNPPIPVGEIYKRWIILGLNKIVRMYSPTSILDIRQGPKEPFRDYVDRFY |
| | Pol: 747-827 | | KTLRAEQASQEVKNWMTETLLVQNANPDCKTILKALGPAATLEEMMTACQGVGGPGHKA |
| | Pol: 683-708 | | RVLAEAMSQMAARASVLSGGELDRWEKIRLRPGGKKKYRLKHIVWASRELERFAVNPGL |
| | Nef: 117-148 | | LETPPVVAKEIVASCDKCQLKGEAMHGQVDCSPGIWQLDCTHLEGKIILVAVHVASGYI |
| | RAKR | | EAEVIPAETGQETAYFLLKLAGRWPVKTKEKVYLAWVPAHKGIGGNEQVDKLVSTQGYF |
| | F2A linker | | PDWQNYTPGPGTRYPLTFGWCFKLVPVRAKRAPVKQTLNFDLLKLAGDVESNPGPFPQI |
| | Pol: 56-117 | | TLWQRPLVTIKIGGQLKEALLDTGADDTVLEEMNLPGRWKPKMIGGIGGFIKVRQYDQE |
| | Nef: 64-99 | | EVGFPVKPQVPLRPMTFKGALDLSHFLREKGGLEGLIPKFKLPIQKETWETWWTEYWQA |
| | LI | | TWIPEWEFVNTPPLVKLWYQLEKEPIVGAETFYVDGAANRETKWGFTTPDKKHQKEPPF |
| | Pol: 542-606 | | LWMGYELHPDKWTVQPIVLPEKDSWTVNDIQKLVGKLNWASQIYPGIKVITKIQNFRVY |
| | Pol: 367-431 | | YRDSRDPLWKGPAKLLWKGEGAVVIQDNSDIKVVPRRKAKIIRDYGKQMAGDDCVASRQ |
| | Pol: 932-1003 | | DEDGTVLVGPTPVNIIGRNLLTQIGCTLNFPISPIETVPVKLKPGMDGPKVKQWPLTEE |
| | Pol: 129-320 | | KIKALVEICTEMEKEGKISKIGPENPYNTPVFAIKKKDSTKWRKLVDFRELNKRTQDFW |
| | | | EVQLGIPHPAGLKKKKSVTVLDVGDAYFSVPLDKDFRKYTAFTIPSINNETPGIRYQYN |
| | | | VLPQGWKGSPAIFQSSMT |
| 209 | M | 1030 | MLSPRTLNAWVKVIEEKAFSPEVIPMFTALSEGATPHDLNTMLNTIGGHQAAMQMLKDT |
| | Gag: 147-369 | | INEEAAEWDRVHPVHAGPVAPGQMRDPRGSDIAGSTSTLQEQIAWMTNNPPIPVGDIYK |
| | Pol: 747-827 | | RWIIMGLNKIVRMYSPVSILDIKQGPKEPFRDYVDRFYRTLRAEQASQDVKNWMTETLL |
| | Gag: 1-53 | | VQNSNPDCKTILKALGPGATLEEMMSACQGVGGPSHKARVLAEAMCQVAKEIVACCDKC |
| | AA | | QLKGEAIHGQVDCSPGVWQLDCTHLEGKVILVAVHVASGYIEAEIIPTETGQETAYFIL |
| | Pol: 840-920 | | KLAGRWPVTTMAARASILSGGKLDKWEKIRLRPGGRKKYKLKHLVWASRELERFALNPG |
| | Nef: 117-148 | | LLETAAAVKAACWWAGVKQEFGIPYNTQSQGVVESMNNELKKIIGQIRDQAEHLKTAVQ |
| | LIK | | MAVLIHNFKRKGGIGEYSAGERIIDIIATQGFFPDWQNYTPGPGIRFPLTFGWCFKLVP |
| | Pol: 683-708 | | LLIKKEKIYLAWVPAHKGIGGNEQIDKLVSRAKRAPVKQTLNFDLLKLAGDVESNPGPM |
| | RAKR | | LPQITLWQRPIVTIKIGGQIKEALLDTGADDTVLEDMNLPGKWKPKMIGGIGGFIKVKQ |
| | F2A linker | | YDQWGLTTPDKKHQKDPPFLWMGYELHPDRWTVQPIELPEKESWTVNDIQKLIGKLNWA |
| | M | | SQIYAGIKVAAQEEEEVGFPVRPQVPLRPMTYKGALDLSHFLKEKGGLEGITKLQNFRV |
| | Pol: 56-117 | | YYRDNRDPLWKGPARLLWKGEGAVVIQDNSEIKVVPRRKVKIIRDYGKRMAGDDCVAGR |
| | Pol: 367-431 | | QDEDPKFRLPIQKETWDTWWTDYWQATWIPEWEFTNTPPLVKLWYQLETEPIAGVETFY |
| | AA | | VDGASNRETKAAGTVLIGPTPVNIIGRNLLTQLGCTLNFPISPIDTVPVKLKPGMDGPR |
| | QEE | | VKQWPLTEEKIKALIEICTEMEKEGKISRIGPENPYNTPIFAIKKKDGTKWRKLVDFRE |
| | Nef: 64-99 | | LNKKTQDFWEVQLGIPHPSGLKKKKSVTVLDIGDAYFSVPLDKEFRKYTAFTVPSTNNE |
| | Pol: 932-1003 | | TPGVRYQYNVLPMGWKGSPAIFQCSMT |
| | Pol: 542-606 | | |
| | AA | | |
| | Pol: 129-320 | | |
| 227 | Gag: 147-369 | 1029 | LSPRTLNAWVKVIEEKAFSPEVIPMFTALSEGATPHDLNTMLNTIGGHQAAMQMLKDTI |
| | Pol: 747-827 | | NEEAAEWDRVHPVHAGPVAPGQMRDPRGSDIAGSTSTLQEQIAWMTNNPPIPVGDIYKR |
| | Gag: 1-53 | | WIIMGLNKIVRMYSPVSILDIKQGPKEPFRDYVDRFYRTLRAEQASQDVKNWMTETLLV |
| | AA | | QNSNPDCKTILKALGPGATLEEMMSACQGVGGPSHKARVLAEAMCQVAKEIVACCDKCQ |
| | Pol: 840-920 | | LKGEAIHGQVDCSPGVWQLDCTHLEGKVILVAVHVASGYIEAEIIPTETGQETAYFILK |
| | Nef: 117-148 | | LAGRWPVTTMAARASILSGGKLDKWEKIRLRPGGRKKYKLKHLVWASRELERFALNPGL |
| | LIK | | LETAAAVKAACWWAGVKQEFGIPYNTQSQGVVESMNNELKKIIGQIRDQAEHLKTAVQM |
| | Pol: 683-708 | | AVLIHNFKRKGGIGEYSAGERIIDIIATQGFFPDWQNYTPGPGIRFPLTFGWCFKLVPL |
| | RAKR | | LIKKEKIYLAWVPAHKGIGGNEQIDKLVSRAKRAPVKQTLNFDLLKLAGDVESNPGPML |
| | F2A linker | | PQITLWQRPIVTIKIGGQIKEALLDTGADDTVLEDMNLPGKWKPKMIGGIGGFIKVKQY |
| | M | | DQWGLTTPDKKHQKDPPFLWMGYELHPDRWTVQPIELPEKESWTVNDIQKLIGKLNWAS |
| | Pol: 56-117 | | QIYAGIKVAAQEEEEVGFPVRPQVPLRPMTYKGALDLSHFLKEKGGLEGITKLQNFRVY |
| | Pol: 367-431 | | YRDNRDPLWKGPARLLWKGEGAVVIQDNSEIKVVPRRKVKIIRDYGKRMAGDDCVAGRQ |
| | AA | | DEDPKFRLPIQKETWDTWWTDYWQATWIPEWEFTNTPPLVKLWYQLETEPIAGVETFYV |
| | QEE | | DGASNRETKAAGTVLIGPTPVNIIGRNLLTQLGCTLNFPISPIDTVPVKLKPGMDGPRV |
| | Nef: 64-99 | | KQWPLTEEKIKALIEICTEMEKEGKISRIGPENPYNTPIFAIKKKDGTKWRKLVDFREL |
| | Pol: 932-1003 | | NKKTQDFWEVQLGIPHPSGLKKKKSVTVLDIGDAYFSVPLDKEFRKYTAFTVPSTNNET |
| | Pol: 542-606 | | PGVRYQYNVLPMGWKGSPAIFQCSMT |
| | AA | | |
| | Pol: 129-320 | | |
| 223 | Gag: 147-369 | 1028 | LSPRTLNAWVKVIEEKAFSPEVIPMFTALSEGATPHDLNTMLNTIGGHQAAMQMLKDTI |
| | Pol: 747-827 | | NEEAAEWDRVHPVHAGPVAPGQMRDPRGSDIAGSTSTLQEQIAWMTNNPPIPVGDIYKR |
| | Gag: 1-53 | | WIIMGLNKIVRMYSPVSILDIKQGPKEPFRDYVDRFYRTLRAEQASQDVKNWMTETLLV |
| | AA | | QNSNPDCKTILKALGPGATLEEMMSACQGVGGPSHKARVLAEAMCQVAKEIVACCDKCQ |
| | Pol: 840-920 | | LKGEAIHGQVDCSPGVWQLDCTHLEGKVILVAVHVASGYIEAEIIPTETGQETAYFILK |
| | Nef: 117-148 | | LAGRWPVTTMAARASILSGGKLDKWEKIRLRPGGRKKYKLKHLVWASRELERFALNPGL |
| | LIK | | LETAAAVKAACWWAGVKQEFGIPYNTQSQGVVESMNNELKKIIGQIRDQAEHLKTAVQM |
| | Pol: 683-708 | | AVLIHNFKRKGGIGEYSAGERIIDIIATQGFFPDWQNYTPGPGIRFPLTFGWCFKLVPL |
| | RAKR | | LIKKEKIYLAWVPAHKGIGGNEQIDKLVSRAKRAPVKQTLNFDLLKLAGDVESNPGPLP |
| | F2A linker | | QITLWQRPIVTIKIGGQIKEALLDTGADDTVLEDMNLPGKWKPKMIGGIGGFIKVKQYD |
| | Pol: 56-117 | | QWGLTTPDKKHQKDPPFLWMGYELHPDRWTVQPIELPEKESWTVNDIQKLIGKLNWASQ |
| | Pol: 367-431 | | IYAGIKVAAQEEEEVGFPVRPQVPLRPMTYKGALDLSHFLKEKGGLEGITKLQNFRVYY |
| | AA | | RDNRDPLWKGPARLLWKGEGAVVIQDNSEIKVVPRRKVKIIRDYGKRMAGDDCVAGRQD |
| | QEE | | EDPKFRLPIQKETWDTWWTDYWQATWIPEWEFTNTPPLVKLWYQLETEPIAGVETFYVD |
| | Nef: 64-99 | | GASNRETKAAGTVLIGPTPVNIIGRNLLTQLGCTLNFPISPIDTVPVKLKPGMDGPRVK |
| | Pol: 932-1003 | | QWPLTEEKIKALIEICTEMEKEGKISRIGPENPYNTPIFAIKKKDGTKWRKLVDFRELN |
| | Pol: 542-606 | | KKTQDFWEVQLGIPHPSGLKKKKSVTVLDIGDAYFSVPLDKEFRKYTAFTVPSTNNETP |
| | AA | | GVRYQYNVLPMGWKGSPAIFQCSMT |
| | Pol: 129-320 | | |
| 222 | Gag: 147-369 | 1030 | ISPRTLNAWVKVVEEKAFSPEVIPMFSALSEGATPQDLNTMLNTVGGHQAAMQMLKETI |
| | Pol: 747-827 | | NEEAAEWDRLHPVHAGPIAPGQMREPRGSDIAGTTSTLQEQIGWMTNNPPIPVGEIYKR |
| | Gag: 1-53 | | WIILGLNKIVRMYSPTSILDIRQGPKEPFRDYVDRFYKTLRAEQASQEVKNWMTETLLV |
| | AA | | QNANPDCKTILKALGPAATLEEMMTACQGVGGPGHKARVLAEAMSQPPVVAKEIVASCD |
| | Pol: 840-920 | | KCQLKGEAMHGQVDCSPGIWQLDCTHLEGKIILVAVHVASGYIEAEVIPAETGQETAYF |
| | Nef: 117-148 | | LLKLAGRWPVKTMAARASVLSGGELDRWEKIRLRPGGKKKYRLKHIVWASRELERFAVN |
| | LIK | | PGLLETAATVKAACWWAGIKQEFGIPYNPQSQGVVESMNKELKKIIGQVRDQAEHLKTA |
| | Pol: 683-708 | | VQMAVFIHNFKRKGGIGGYSAGERIVDIIATQGYFPDWQNYTPGPGTRYPLTFGWCFKL |
| | F2A | | VPVKEKVYLAWVPAHKGIGGNEQVDKLVSRAKRAPVKQTLNFDLLKLAGDVESNPGPFP |
| | Pol: 56-117 | | QITLWQRPLVTIKIGGQLKEALLDTGADDTVLEEMNLPGRWKPKMIGGIGGFIKVRQYD |
| | Pol: 367-431 | | QWGFTTPDKKHQKEPPFLWMGYELHPDKWTVQPIVLPEKDSWTVNDIQKLVGKLNWASQ |
| | AA | | IYPGIKVAAQEEEEVGFPVKPQVPLRPMTFKGALDLSHFLREKGGLEGLIITKIQNFRV |
| | QEE | | YYRDSRDPLWKGPAKLLWKGEGAVVIQDNSDIKVVPRRKAKIIRDYGKQMAGDDCVASR |
| | Nef: 64-99 | | QDEDPKFKLPIQKETWETWWTEYWQATWIPEWEFVNTPPLVKLWYQLEKEPIVGAETFY |
| | Pol: 932-1003 | | VDGAANRETKAAGTVLVGPTPVNIIGRNLLTQIGCTLNFPISPIETVPVKLKPGMDGPK |
| | Pol: 542-606 | | VKQWPLTEEKIKALVEICTEMEKEGKISKIGPENPYNTPVFAIKKKDSTKWRKLVDFRE |
| | AA | | LNKRTQDFWEVQLGIPHPAGLKKKKSVTVLDVGDAYFSVPLDKDFRKYTAFTIPSINNE |
| | Pol: 129-320 | | TPGIRYQYNVLPQGWKGSPAIFQSSMT |
| 200 | Pol: 840-920 | 735 | TVKAACWWAGIKQEFGIPYNPQSQGVVESMNKELKKIIGQVRDQAEHLKTAVQMAVFIH |
| | Gag: 147-369 | | NFKRKGGIGGYSAGERIVDIIAISPRTLNAWVKVVEEKAFSPEVIPMFSALSEGATPQD |
| | Pol: 586-606 | | LNTMLNTVGGHQAAMQMLKETINEEAAEWDRLHPVHAGPIAPGQMREPRGSDIAGTTST |
| | AA | | LQEQIGWMTNNPPIPVGEIYKRWIILGLNKIVRMYSPTSILDIRQGPKEPFRDYVDRFY |
| | Pol: 683-708 | | KTLRAEQASQEVKNWMTETLLVQNANPDCKTILKALGPAATLEEMMTACQGVGGPGHKA |
| | RAKR | | RVLAEAMSQKEPIVGAETFYVDGAANRETKAAKEKVYLAWVPAHKGIGGNEQVDKLVSR |
| | F2A linker | | AKRAPVKQTLNFDLLKLAGDVESNPGPLSPRTLNAWVKVIEEKAFSPEVIPMFTALSEG |
| | Gag: 147-369 | | ATPHDLNTMLNTIGGHQAAMQMLKDTINEEAAEWDRVHPVHAGPVAPGQMRDPRGSDIA |
| | Pol: 840-920 | | GSTSTLQEQIAWMTNNPPIPVGDIYKRWIIMGLNKIVRMYSPVSILDIKQGPKEPFRDY |
| | Pol: 586-606 | | VDRFYRTLRAEQASQDVKNWMTETLLVQNSNPDCKTILKALGPGATLEEMMSACQGVGG |
| | AA | | PSHKARVLAEAMCQAVKAACWWAGVKQEFGIPYNTQSQGVVESMNNELKKIIGQIRDQA |
| | Pol: 683-708 | | EHLKTAVQMAVLIHNFKRKGGIGEYSAGERIIDIIATEPIAGVETFYVDGASNRETKAA |
| | | | KEKIYLAWVPAHKGIGGNEQIDKLVS |
| 201 | Pol: 932-1003 | 758 | ITKIQNFRVYYRDSRDPLWKGPAKLLWKGEGAVVIQDNSDIKVVPRRKAKIIRDYGKQM |
| | AAA | | AGDDCVASRQDEDAAAIGTVLVGPTPVNIIGRNLLTQIGCTLNFPISPIETVPVKLKPG |
| | I | | MDGPKVKQWPLTEEKIKALVEICTEMEKEGKISKIGPENPYNTPVFAIKKKDSTKWRKL |
| | Pol: 129-320 | | VDFRELNKRTQDFWEVQLGIPHPAGLKKKKSVTVLDVGDAYFSVPLDKDFRKYTAFTIP |
| | Pol: 747-827 | | SINNETPGIRYQYNVLPQGWKGSPAIFQSSMTVAKEIVASCDKCQLKGEAMHGQVDCSP |
| | QEE | | GIWQLDCTHLEGKIILVAVHVASGYIEAEVIPAETGQETAYFLLKLAGRWPVKTQEEEE |
| | Nef: 64-76 | | VGFPVKPQVPLRAKRAPVKQTLNFDLLKLAGDVESNPGPVAKEIVACCDKCQLKGEAIH |
| | RAKR | | GQVDCSPGVWQLDCTHLEGKVILVAVHVASGYIEAEIIPTETGQETAYFILKLAGRWPV |
| | F2A linker | | TTITKLQNFRVYYRDNRDPLWKGPARLLWKGEGAVVIQDNSEIKVVPRRKVKIIRDYGK |
| | Pol: 747-827 | | RMAGDDCVAGRQDEDGTVLIGPTPVNIIGRNLLTQLGCTLNFPISPIDTVPVKLKPGMD |
| | Pol: 932-1003 | | GPRVKQWPLTEEKIKALIEICTEMEKEGKISRIGPENPYNTPIFAIKKKDGTKWRKLVD |
| | Pol: 129-320 | | FRELNKKTQDFWEVQLGIPHPSGLKKKKSVTVLDIGDAYFSVPLDKEFRKYTAFTVPST |
| | KIL | | NNETPGVRYQYNVLPMGWKGSPAIFQCSMTKILQEEEEVGFPVRPQVPL |
| | QEE | | |
| | Nef: 64-76 | | |
| 202 | Pol: 840-920 | 747 | TVKAACWWAGIKQEFGIPYNPQSQGVVESMNKELKKIIGQVRDQAEHLKTAVQMAVFIH |
| | Gag: 147-369 | | NFKRKGGIGGYSAGERIVDIIAISPRTLNAWVKVVEEKAFSPEVIPMFSALSEGATPQD |
| | Pol: 586-606 | | LNTMLNTVGGHQAAMQMLKETINEEAAEWDRLHPVHAGPIAPGQMREPRGSDIAGTTST |
| | AA | | LQEQIGWMTNNPPIPVGEIYKRWIILGLNKIVRMYSPTSILDIRQGPKEPFRDYVDRFY |
| | Pol: 683-708 | | KTLRAEQASQEVKNWMTETLLVQNANPDCKTILKALGPAATLEEMMTACQGVGGPGHKA |
| | RAKR | | RVLAEAMSQKEPIVGAETFYVDGAANRETKAAKEKVYLAWVPAHKGIGGNEQVDKLVSR |
| | F2A linker | | AKRAPVKQTLNFDLLKLAGDVESNPGPITKIQNFRVYYRDSRDPLWKGPAKLLWKGEGA |
| | Pol: 932-1003 | | VVIQDNSDIKVVPRRKAKIIRDYGKQMAGDDCVASRQDEDAAAIGTVLVGPTPVNIIGR |
| | AAA | | NLLTQIGCTLNFPISPIETVPVKLKPGMDGPKVKQWPLTEEKIKALVEICTEMEKEGKI |
| | I | | SKIGPENPYNTPVFAIKKKDSTKWRKLVDFRELNKRTQDFWEVQLGIPHPAGLKKKKSV |
| | Pol: 129-320 | | TVLDVGDAYFSVPLDKDFRKYTAFTIPSINNETPGIRYQYNVLPQGWKGSPAIFQSSMT |
| | Pol: 747-827 | | VAKEIVASCDKCQLKGEAMHGQVDCSPGIWQLDCTHLEGKIILVAVHVASGYIEAEVIP |
| | QEE | | AETGQETAYFLLKLAGRWPVKTQEEEEVGFPVKPQVPL |
| | Nef: 64-76 | | |
| 203 | Gag: 147-369 | 746 | LSPRTLNAWVKVIEEKAFSPEVIPMFTALSEGATPHDLNTMLNTIGGHQAAMQMLKDTI |
| | Pol: 840-920 | | NEEAAEWDRVHPVHAGPVAPGQMRDPRGSDIAGSTSTLQEQIAWMTNNPPIPVGDIYKR |
| | Pol: 586-606 | | WIIMGLNKIVRMYSPVSILDIKQGPKEPFRDYVDRFYRTLRAEQASQDVKNWMTETLLV |
| | AA | | QNSNPDCKTILKALGPGATLEEMMSACQGVGGPSHKARVLAEAMCQAVKAACWWAGVKQ |
| | Pol: 683-708 | | EFGIPYNTQSQGVVESMNNELKKIIGQIRDQAEHLKTAVQMAVLIHNFKRKGGIGEYSA |
| | RAKR | | GERIIDIIATEPIAGVETFYVDGASNRETKAAKEKIYLAWVPAHKGIGGNEQIDKLVSR |
| | F2A linker | | AKRAPVKQTLNFDLLKLAGDVESNPGPVAKEIVACCDKCQLKGEAIHGQVDCSPGVWQL |
| | Pol: 747-827 | | DCTHLEGKVILVAVHVASGYIEAEIIPTETGQETAYFILKLAGRWPVTTITKLQNFRVY |
| | Pol: 932-1003 | | YRDNRDPLWKGPARLLWKGEGAVVIQDNSEIKVVPRRKVKIIRDYGKRMAGDDCVAGRQ |
| | Pol: 129-320 | | DEDGTVLIGPTPVNIIGRNLLTQLGCTLNFPISPIDTVPVKLKPGMDGPRVKQWPLTEE |
| | KIL | | KIKALIEICTEMEKEGKISRIGPENPYNTPIFAIKKKDGTKWRKLVDFRELNKKTQDFW |
| | QEE | | EVQLGIPHPSGLKKKKSVTVLDIGDAYFSVPLDKEFRKYTAFTVPSTNNETPGVRYQYN |
| | Nef: 64-76 | | VLPMGWKGSPAIFQCSMTKILQEEEEVGFPVRPQVPL |
| 204 | Pol: 840-920 | 717 | TVKAACWWAGIKQEFGIPYNPQSQGVVESMNKELKKIIGQVRDQAEHLKTAVQMAVFIH |
| | Gag: 147-369 | | NFKRKGGIGGYSAGERIVDIIAISPRTLNAWVKVVEEKAFSPEVIPMFSALSEGATPQD |
| | Pol: 586-606 | | LNTMLNTVGGHQAAMQMLKETINEEAAEWDRLHPVHAGPIAPGQMREPRGSDIAGTTST |
| | Pol: 747-827 | | LQEQIGWMTNNPPIPVGEIYKRWIILGLNKIVRMYSPTSILDIRQGPKEPFRDYVDRFY |
| | Pol: 683-708 | | KTLRAEQASQEVKNWMTETLLVQNANPDCKTILKALGPAATLEEMMTACQGVGGPGHKA |
| | Nef: 64-76 | | RVLAEAMSQKEPIVGAETFYVDGAANRETKVAKEIVASCDKCQLKGEAMHGQVDCSPGI |
| | Pol: 932-1003 | | WQLDCTHLEGKIILVAVHVASGYIEAEVIPAETGQETAYFLLKLAGRWPVKTKEKVYLA |
| | AAA | | WVPAHKGIGGNEQVDKLVSQEEEEVGFPVKPQVPLITKIQNFRVYYRDSRDPLWKGPAK |
| | Pol: 129-320 | | LLWKGEGAVVIQDNSDIKVVPRRKAKIIRDYGKQMAGDDCVASRQDEDAAAIGTVLVGP |
| | | | TPVNIIGRNLLTQIGCTLNFPISPIETVPVKLKPGMDGPKVKQWPLTEEKIKALVEICT |
| | | | EMEKEGKISKIGPENPYNTPVFAIKKKDSTKWRKLVDFRELNKRTQDFWEVQLGIPHPA |
| | | | GLKKKKSVTVLDVGDAYFSVPLDKDFRKYTAFTIPSINNETPGIRYQYNVLPQGWKGSP |
| | | | AIFQSSMT |
| 205 | Gag: 147-369 | 716 | LSPRTLNAWVKVIEEKAFSPEVIPMFTALSEGATPHDLNTMLNTIGGHQAAMQMLKDTI |
| | Pol: 747-827 | | NEEAAEWDRVHPVHAGPVAPGQMRDPRGSDIAGSTSTLQEQIAWMTNNPPIPVGDIYKR |
| | Pol: 683-708 | | WIIMGLNKIVRMYSPVSILDIKQGPKEPFRDYVDRFYRTLRAEQASQDVKNWMTETLLV |
| | Pol: 586-606 | | QNSNPDCKTILKALGPGATLEEMMSACQGVGGPSHKARVLAEAMCQVAKEIVACCDKCQ |
| | Pol: 932-1003 | | LKGEAIHGQVDCSPGVWQLDCTHLEGKVILVAVHVASGYIEAEIIPTETGQETAYFILK |
| | Pol: 840-920 | | LAGRWPVTTKEKIYLAWVPAHKGIGGNEQIDKLVSTEPIAGVETFYVDGASNRETKITK |
| | Nef: 64-76 | | LQNFRVYYRDNRDPLWKGPARLLWKGEGAVVIQDNSEIKVVPRRKVKIIRDYGKRMAGD |
| | Pol: 129-320 | | DCVAGRQDEDAVKAACWWAGVKQEFGIPYNTQSQGVVESMNNELKKIIGQIRDQAEHLK |
| | | | TAVQMAVLIHNFKRKGGIGEYSAGERIIDIIAQEEEEVGFPVRPQVPLGTVLIGPTPVN |
| | | | IIGRNLLTQLGCTLNFPISPIDTVPVKLKPGMDGPRVKQWPLTEEKIKALIEICTEMEK |
| | | | EGKISRIGPENPYNTPIFAIKKKDGTKWRKLVDFRELNKKTQDFWEVQLGIPHPSGLKK |
| | | | KKSVTVLDIGDAYFSVPLDKEFRKYTAFTVPSTNNETPGVRYQYNVLPMGWKGSPAIFQ |
| | | | CSMTKIL |

### Signal or Leader Sequences

In various embodiments, the fusion polypeptides and/or compound fusion polypeptides comprise a signal sequence or signal peptide, e.g., to direct intracellular trafficking of the fusion polypeptide or compound fusion polypeptide to a proteasomal or lysosomal compartment. In various embodiments, fusion polypeptide or compound fusion polypeptide comprises a signal sequence at the N-terminus and/or the C-terminus. In some embodiments, the fusion polypeptide or compound fusion polypeptide comprises an N-terminal signal peptide or leader sequence. In various embodiments, the signal peptide or leader sequence is from a source protein selected from a serum protein, a cytokine, a chemokine, a chaperone protein, an invariant protein, and a protein that directs proteins to the lysosomal compartment. In some embodiments, the signal peptide or leader sequence is from a source protein selected from colony stimulating factor 2 (CSF2, GM-CSF), tissue type plasminogen activator (PLAT, t-PA), C-C motif chemokine ligand 7 (CCL7, MCP-3), C-X-C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2). In certain embodiments, the fusion polypeptide comprises N-terminal and C-terminal signal sequences from LAMP-1, e.g, SEQ ID NOs: 125 and 126, respectively. In various embodiments, the signal peptide or leader sequence is selected from an amino acid sequence of any one of SEQ ID NOs: 115-126, or a nucleic acid sequence that is at least 95%, 96%, 97%, 98%, or 99% identical to any one of SEQ ID NOs: 115-126. Illustrative signal sequences that can be used in the present fusion polypeptides and compound fusion polypeptides are provided in Table G.

TABLE G
signal sequences

| SEQ | source | |
|---|---|---|
| ID | protein | |
| NO: | name | SEQUENCE |
| 115 | CD74 | MHRRRSRSCREDQKPV |
| 116 | VSV-G | MKCLLYLAFLFIGVNC |
| 117 | albumin | KWVTFISLLELESSAYS |
| 118 | calreticulin | MLLSVPLLLGLLGLAVA |
| 119 | CSF2, GM-CSF | MWLQSLLLLGTVACSISV |
| 120 | CXCL10, IP-10 | MNQTAILICCLIFLTLSGIQG |
| 121 | PLAT, t-PA | MDAMKRGLCCVLLLCGAVEVSAR |
| 122 | β-catenin | MRKAAVSHWQQQSYLDSGIHSGATTTAPSLS |
| 123 | ubiquitin | MQIFVKTLTGKTITLEVEPSDTIENVKAKIQ |
| | | DKEGIPPDQQRLIFAGKQLEDGRTLSDYNIQ |
| | | KESTLHLVLRLRGG |
| 124 | CCL7, MCP-3 | MKASAALLCLLLTAAAFSPQGLA |
| 125 | LAMP-1 | MAPRSARRPLLLLLLLLLLGLMHCASAAMEM |
| | N-terminal | VKNGNGTACIMANFSAAFSVNYDTKSGPKNM |
| | | TLDLPSDATVVLNRSSCGKENTSDPSLVIAF |
| | | GRGHTLTLNFTRNATRYSVQLMSFVYNLSDT |
| | | HLFPNASSKEIKTVESITDIRADIDKKYRCV |
| | | SGTQVHMNNVTVTLHDATIQAYLSNSSFSRG |
| | | ETRCEQDRPSPTTAPPAPPSPSPSPVPKSPS |
| | | VDKYNVSGTNGTCLLASMGLQLNLTYERKDN |
| | | TTVTRLLNINPNKTSASGSCGAHLVTLELHS |
| | | EGTTVLLFQFGMNASSSRFELQGIQLNTlLP |
| | | DARDPAFKAANGSLRALQATVGNSYKCNAEE |
| | | HVRVTKAFSVNIFKVWVQAFKVEGGQFGSVE |
| | | ECLLDENSLEDI |
| 126 | LAMP-1 | GSEFTLIPIAVGGALAGLVIVLIAYLVGRKR |
| | C-terminal | SHAGYQTI |

### 3. Polynucleotides Encoding the Fusion Polypeptides or Compound Fusion Polypeptides

Provided are polynucleotides encoding the fusion polypeptides or the compound fusion polypeptides, described herein, vectors comprising such polynucleotides, and host cells (e.g., human cells, mammalian cells, yeast cells, plant cells, insect cells, bacterial cells, e.g., E. coli) comprising such polynucleotides or expression vectors. Provided herein are polynucleotides comprising nucleotide sequence(s) encoding any of the fusion polypeptides or compound fusion polypeptides provided herein, as well as expression cassettes and vector(s) comprising such polynucleotide sequences, e.g., expression vectors for their efficient expression in host cells, e.g., mammalian cells. In various embodiments, the polynucleotide is a DNA, a cDNA, an mRNA, a self-amplifying RNA (SAM), a self-replicating RNA, or a self-amplifying replicon RNA (RepRNA). In some embodiments, the polynucleotide comprises an alphavirus self-replicating or self-amplifying replicon RNA (RepRNA). Self-replicating RNA and self-amplifying replicon RNA as modes of vaccine delivery are described, e.g., by Ballesteros-Briones, et al., Curr Opin Virol. (2020) 44:145-153; Bloom, et al., Gene Ther. (2020) 22:1-13; Lundstrom, Int. J. Mol. Sci. (2020) 21:5130; Moyo, et al., Mol Ther Methods Clin Dev. (2018) 12:32-46; Tews, et al., Methods Mol Biol. (2017) 1499:15-35; Demoulins, et al., Methods Mol Biol. (2017) 1499:37-75; Englezou, et al., Mol Ther Nucleic Acids. (2018) 12:118-134; McCollough, et al., Vaccines (Basel). (2014) 2 (4): 735-54; and McCollough, et al., Mol Ther Nucleic Acids. (2014) 3: e173.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include without limitation, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired viral expression vector or host cell.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid” encoding a polypeptide segment or a fusion polypeptide or a compound fusion polypeptide refers to one or more nucleic acid molecules encoding such polypeptide segments or fusion polypeptides or compound fusion polypeptides, including such nucleic acid molecule(s) in a single vector or separate vectors, and such nucleic acid molecule(s) present at one or more locations in a host cell.

A “polynucleotide variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences described herein and evaluating one or more biological activities of the encoded polypeptide as described herein and/or using any of a number of techniques well known in the art.

In some embodiments, the nucleic acid molecule is codon-biased to enhance expression in a desired host cell, e.g., in human cells, mammalian cells, yeast cells, plant cells, insect cells, or bacterial cells, e.g., E. coli cells. Accordingly, provided are polynucleotides encoding a fusion polypeptide or a compound fusion polypeptide, described herein, wherein the polynucleotides are codon-biased, comprise replacement heterologous signal sequences, and/or have mRNA instability elements eliminated. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the fusion polypeptides or compound fusion polypeptides comprising HIV-1 polypeptide segments from desired viral expression vectors and/or in desired host cells is provided, e.g., at kazusa.or.jp/codon/; and genscript.com/tools/codon-frequency-table.

In some embodiments, the polynucleotide encoding a fusion polypeptide or a compound fusion polypeptide, as described herein, which has a nucleic acid sequence of any one of SEQ ID NOs: 130-167, 210-219 and 225-226, or is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from SEQ ID NOs: 130-167, 210-219 and 225-226, as provided in Table H. In some embodiments, the polynucleotide encoding a fusion polypeptide or a compound fusion polypeptide, as described herein, which has a nucleic acid sequence of any one of SEQ ID NOs: 139, 140, 142, 143, 145, 146, 148, 149, 150, 152, 155, 158, 225 and 226, or is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from SEQ ID NOs: 139, 140, 142, 143, 145, 146, 148, 149, 150, 152, 155, 158, 225 and 226, as provided in Table H. Table H discloses “AAA” as SEQ ID NO: 48, “LIK” as SEQ ID NO: 53, “AAY” as SEQ ID NO: 49, “SEG” as SEQ ID NO: 55, “QEE” as SEQ ID NO: 51, “RAKR” as SEQ ID NO: 60, “KIL” as SEQ ID NO: 52 and “PPV” as SEQ ID NO: 54. In some embodiments, the polynucleotide encoding a fusion polypeptide or a compound fusion polypeptide, as described herein, which has a nucleic acid sequence of SEQ ID NO: 139, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 139. In some embodiments, the polynucleotide encoding a fusion polypeptide or a compound fusion polypeptide, as described herein, which has a nucleic acid sequence of SEQ ID NO: 142, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 142. In some embodiments, the polynucleotide encoding a fusion polypeptide or a compound fusion polypeptide, as described herein, which has a nucleic acid sequence of SEQ ID NO: 145, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 145. In some embodiments, the polynucleotide encoding a fusion polypeptide or a compound fusion polypeptide, as described herein, which has a nucleic acid sequence of SEQ ID NO: 148, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 148. In some embodiments, the polynucleotide encoding a fusion polypeptide or a compound fusion polypeptide, as described herein, which has a nucleic acid sequence of SEQ ID NO: 150, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 150. In some embodiments, the polynucleotide encoding a fusion polypeptide or a compound fusion polypeptide, as described herein, which has a nucleic acid sequence of SEQ ID NO: 152, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 152. In some embodiments, the polynucleotide encoding a fusion polypeptide or a compound fusion polypeptide, as described herein, which has a nucleic acid sequence of SEQ ID NO: 155, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 155. In some embodiments, the polynucleotide encoding a fusion polypeptide or a compound fusion polypeptide, as described herein, which has a nucleic acid sequence of SEQ ID NO: 158, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 158. In some embodiments, the polynucleotide encoding a fusion polypeptide or a compound fusion polypeptide, as described herein, which has a nucleic acid sequence of SEQ ID NO: 225, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 225. In some embodiments, the polynucleotide encoding a fusion polypeptide or a compound fusion polypeptide, as described herein, which has a nucleic acid sequence of SEQ ID NO: 226, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to SEQ ID NO: 226.

TABLE H
Illustrative Polynucleotides Encoding Fusion Polypeptides

| SEQ | Encoded | |
|---|---|---|
| ID | Polypeptide | |
| NO: | Segments | Nucleic Acid Sequences |
| 130 | Pol:840-920 | ACAGTGAAGGCAGCTTGTTGGTGGGCCGGAATTAAACAGGAGTTTGGCATCCCTTATAATCCTCAGTCTCAG |
| | Gag:147-369 | GGAGTGGTGGAGTCTATGAACAAGGAGCTGAAGAAGATCATCGGCCAGGTGAGAGATCAGGCAGAACATCTG |
| | Gag:1-53 | AAGACAGCAGTGCAGATGGCCGTGTTTATCCACAACTTCAAGAGGAAGGGCGGCATTGGAGGATATAGCGCA |
| | PPV | GGAGAAAGAATCGTGGACATCATCGCCATCTCTCCTAGAACACTGAACGCTTGGGTGAAAGTGGTGGAGGAG |
| | Pol:747-827 | AAAGCCTTTAGCCCAGAAGTGATCCCTATGTTCTCAGCTCTGTCAGAAGGAGCTACACCTCAGGATCTGAAC |
| | Pol:683-708 | ACCATGCTGAATACCGTGGGAGGACATCAGGCAGCTATGCAGATGCTGAAGGAGACAATTAACGAGGAAGCA |
| | Nef:117-148 | GCCGAGTGGGATAGACTGCATCCAGTGCACGCAGGACCTATTGCTCCAGGACAGATGAGAGAGCCTAGAGGA |
| | | AGCGATATTGCCGGCACAACATCTACACTGCAGGAACAGATCGGTTGGATGACCAACAATCCTCCTATCCCA |
| | | GTGGGCGAAATCTACAAACGCTGGATCATCCTGGGCCTGAATAAGATCGTGAGAATGTACAGCCCCACAAGC |
| | | ATCCTGGATATCAGACAGGGACCTAAGGAACCTTTCAGGGATTACGTGGACCGGTTCTACAAGACACTGAGA |
| | | GCAGAACAGGCATCTCAGGAGGTGAAGAATTGGATGACCGAGACACTGCTGGTGCAGAACGCTAATCCAGAT |
| | | TGCAAGACCATTCTGAAAGCTCTGGGACCAGCAGCTACACTGGAAGAGATGATGACAGCTTGTCAGGGAGTG |
| | | GGAGGACCAGGACATAAAGCTAGAGTGCTGGCAGAAGCTATGTCTCAGATGGCAGCTAGAGCTTCAGTGCTG |
| | | TCAGGAGGAGAACTCGATAGGTGGGAGAAGATCAGACTGAGACCAGGAGGCAAGAAGAAGTACAGACTGAAG |
| | | CACATCGTGTGGGCTTCTAGAGAACTGGAGAGATTTGCCGTGAATCCAGGACTCCTGGAAACACCTCCAGTG |
| | | GTGGCTAAAGAGATTGTGGCTTCTTGCGATAAGTGCCAGCTGAAAGGAGAGGCTATGCACGGACAGGTGGAT |
| | | TGTTCTCCAGGAATTTGGCAGCTGGATTGTACACACCTGGAGGGAAAGATTATTCTGGTGGCAGTGCACGTG |
| | | GCATCAGGATATATTGAGGCCGAAGTGATTCCAGCAGAAACAGGACAGGAGACAGCTTACTTTCTGCTCAAA |
| | | CTGGCAGGTCGCTGGCCAGTGAAAACCAAGGAGAAGGTGTACCTGGCTTGGGTGCCAGCTCATAAAGGAATT |
| | | GGCGGAAACGAGCAGGTGGATAAACTGGTGTCTACACAGGGCTACTTCCCAGATTGGCAGAATTACACACCA |
| | | GGACCAGGCACAAGATATCCTCTGACATTCGGTTGGTGTTTCAAGCTGGTGCCCGTG |
| 131 | Pol:840-920 | ACAGTGAAAGCAGCTTGTTGGTGGGCAGGAATCAAGCAGGAGTTTGGCATCCCTTACAATCCTCAGTCTCAG |
| | Gag:147-369 | GGAGTGGTGGAATCTATGAACAAGGAGCTGAAGAAGATCATCGGCCAGGTGAGAGATCAGGCAGAACATCTG |
| | Gag:1-53 | AAGACAGCAGTGCAGATGGCAGTGTTTATCCACAATTTCAAGAGAAAGGGCGGCATTGGCGGATATAGCGCC |
| | PPV | GGAGAGAGAATCGTGGATATCATCGCCATCTCTCCTAGAACACTGAACGCTTGGGTGAAAGTGGTGGAAGAG |
| | Pol:747-827 | AAAGCCTTCTCTCCAGAGGTGATCCCTATGTTTAGCGCTCTGTCAGAAGGAGCTACACCTCAGGATCTGAAT |
| | Pol:683-708 | ACCATGCTGAATACCGTGGGCGGACATCAGGCAGCTATGCAGATGCTGAAAGAGACAATCAACGAAGAAGCA |
| | Nef:117-148 | GCCGAGTGGGATAGACTGCATCCAGTGCACGCAGGACCTATTGCTCCAGGACAGATGAGAGAACCTAGAGGA |
| | | TCAGACATTGCCGGAACAACATCTACACTGCAGGAGCAGATCGGTTGGATGACAAACAACCCTCCAATCCCA |
| | | GTGGGAGAGATCTACAAGAGATGGATCATCCTGGGCCTGAATAAGATCGTGAGAATGTACAGCCCCACAAGC |
| | | ATCCTGGATATCAGACAGGGACCTAAGGAGCCTTTCAGAGATTACGTGGACAGGTTCTACAAGACCCTGAGA |
| | | GCAGAACAGGCTTCTCAGGAGGTGAAAAATTGGATGACCGAAACACTGCTGGTGCAGAACGCTAATCCCGAT |
| | | TGCAAGACCATCCTGAAAGCTCTGGGACCAGCAGCTACACTGGAAGAGATGATGACAGCTTGTCAGGGAGTG |
| | | GGAGGACCAGGACATAAAGCTAGAGTGCTGGCAGAAGCTATGTCTCAGATGGCAGCTAGAGCTTCAGTGCTG |
| | | TCAGGAGGAGAACTCGATAGATGGGAAAAGATCAGACTGAGACCAGGAGGAAAGAAGAAGTACAGGCTGAAG |
| | | CACATCGTCTGGGCTTCTAGAGAACTGGAGAGATTTGCCGTGAATCCAGGACTCCTGGAAACACCTCCAGTG |
| | | GTGGCTAAAGAGATTGTGGCTTCTTGCGACAAGTGTCAGCTGAAAGGAGAGGCTATGCACGGACAGGTGGAT |
| | | TGTTCTCCAGGAATTTGGCAGCTGGATTGCACACATCTGGAAGGAAAGATTATTCTGGTGGCAGTGCACGTG |
| | | GCATCTGGATATATCGAGGCCGAGGTGATTCCAGCCGAAACAGGACAGGAAACAGCCTACTTTCTCCTGAAA |
| | | CTGGCAGGTAGGTGGCCAGTGAAGACAAAGGAGAAGGTGTACCTGGCTTGGGTGCCAGCCCATAAAGGAATT |
| | | GGAGGCAATGAGCAGGTGGATAAACTGGTGTCAACACAGGGCTACTTCCCAGATTGGCAGAATTACACCCCA |
| | | GGACCAGGAACAAGATATCCTCTGACATTCGGTTGGTGCTTTAAACTGGTGCCCGTG |
| 132 | Gag:147-369 | CTGTCTCCTAGAACACTGAACGCTTGGGTGAAGGTGATCGAAGAGAAGGCCTTTAGCCCAGAAGTGATCCCT |
| | Pol:747-827 | ATGTTCACAGCTCTGTCAGAAGGAGCTACACCTCATGACCTGAACACCATGCTGAATACCATCGGAGGACAT |
| | Gag:1-53 | CAGGCAGCTATGCAGATGCTGAAGGATACCATCAACGAAGAAGCAGCCGAGTGGGATAGAGTGCATCCAGTG |
| | AA | CACGCAGGACCAGTGGCTCCAGGACAGATGAGAGATCCTAGAGGAAGCGATATTGCCGGCTCTACATCTACA |
| | Pol:840-920 | CTGCAGGAACAGATCGCTTGGATGACCAACAATCCTCCTATCCCAGTGGGCGATATCTACAAACGCTGGATC |
| | Nef:117-148 | ATCATGGGCCTGAACAAGATCGTGAGGATGTACAGCCCAGTGTCTATCCTGGATATCAAGCAGGGCCCTAAA |
| | LIK | GAACCTTTCAGGGATTACGTGGACCGGTTCTACAGAACACTGAGAGCAGAACAGGCCTCACAGGATGTGAAG |
| | Pol:683-708 | AATTGGATGACCGAGACACTGCTGGTGCAGAACAGCAACCCCGATTGCAAGACCATTCTGAAAGCTCTGGGA |
| | | CCAGGAGCTACACTGGAAGAGATGATGTCAGCTTGTCAGGGAGTGGGAGGACCATCTCATAAAGCTAGAGTG |
| | | CTGGCCGAAGCTATGTGTCAGGTGGCTAAAGAGATCGTGGCTTGTTGCGACAAGTGTCAGCTGAAAGGAGAG |
| | | GCTATTCACGGACAGGTGGATTGTTCTCCAGGAGTCTGGCAGCTGGATTGTACACACCTGGAGGGAAAGGTG |
| | | ATTCTGGTGGCAGTGCACGTGGCATCAGGATATATTGAGGCCGAGATCATTCCTACAGAAACAGGACAGGAG |
| | | ACCGCTTACTTCATCCTGAAACTGGCAGGTAGGTGGCCAGTGACAACAATGGCAGCTAGAGCTTCTATCCTG |
| | | AGCGGAGGAAAACTCGACAAGTGGGAGAAGATCAGACTGAGACCAGGAGGCAGAAAGAAGTACAAGCTGAAG |
| | | CATCTCGTCTGGGCTTCTAGAGAGCTGGAAAGATTCGCTCTGAATCCAGGTCTGCTGGAAACAGCAGCAGCA |
| | | GTGAAAGCAGCTTGTTGGTGGGCAGGAGTGAAACAGGAATTTGGCATCCCTTACAATACACAGTCTCAGGGA |
| | | GTGGTGGAGAGCATGAACAACGAGCTGAAGAAGATCATCGGCCAGATCAGAGATCAGGCAGAACATCTGAAG |
| | | ACAGCAGTGCAGATGGCAGTGCTGATCCACAACTTCAAGAGGAAGGGCGGAATCGGAGAATATAGCGCCGGC |
| | | GAGAGAATTATCGATATCATCGCCACACAGGGCTTTTTCCCAGATTGGCAGAACTATACACCAGGACCAGGA |
| | | ATCAGGTTCCCTCTGACATTCGGTTGGTGTTTCAAGCTGGTGCCTCTGCTGATCAAGAAGGAGAAAATCTAT |
| | | CTGGCTTGGGTGCCAGCTCACAAAGGAATTGGCGGAAACGAGCAGATCGATAAGCTGGTGTCT |
| 133 | Gag:147-369 | CTGTCTCCTAGAACACTGAACGCTTGGGTGAAGGTGATTGAGGAGAAAGCCTTCAGCCCAGAAGTGATCCCT |
| | Pol:747-827 | ATGTTTACAGCCCTGAGCGAAGGAGCTACACCTCACGATCTGAATACCATGCTGAATACAATCGGCGGACAT |
| | Gag:1-53 | CAGGCAGCCATGCAGATGCTGAAGGATACAATCAACGAAGAAGCAGCCGAGTGGGATAGAGTGCATCCAGTG |
| | AA | CACGCAGGACCAGTGGCTCCAGGACAGATGAGAGATCCTAGAGGAAGCGATATCGCCGGATCTACATCTACA |
| | Pol:840-920 | CTGCAGGAACAGATCGCTTGGATGACAAATAACCCCCCTATCCCAGTGGGAGATATCTATAAGCGCTGGATC |
| | Nef:117-148 | ATCATGGGCCTGAACAAGATCGTGAGGATGTACAGCCCAGTGAGCATCCTGGATATCAAGCAGGGACCTAAG |
| | LIK | GAGCCTTTCAGAGATTACGTGGACAGGTTCTACAGAACCCTGAGAGCAGAACAGGCTTCTCAGGACGTGAAG |
| | Pol:683-708 | AATTGGATGACCGAAACACTGCTGGTGCAGAATAGCAACCCCGATTGCAAGACCATCCTGAAAGCTCTGGGA |
| | | CCAGGAGCTACACTGGAAGAAATGATGAGCGCTTGTCAGGGAGTGGGAGGACCATCTCATAAGGCTAGAGTG |
| | | CTGGCAGAAGCTATGTGTCAGGTGGCTAAGGAGATTGTGGCTTGTTGCGACAAGTGTCAGCTGAAAGGAGAG |
| | | GCTATTCACGGACAGGTGGATTGTTCTCCAGGAGTCTGGCAGCTGGATTGTACACATCTGGAGGGAAAAGTG |
| | | ATTCTGGTGGCAGTGCACGTGGCATCAGGATATATTGAGGCCGAAATCATCCCTACAGAGACAGGACAGGAG |
| | | ACAGCCTACTTTATCCTGAAACTGGCAGGCAGATGGCCAGTGACAACAATGGCAGCTAGAGCTTCTATCCTG |
| | | AGCGGAGGAAAGCTGGATAAGTGGGAAAAGATCAGACTGAGACCAGGAGGAAGGAAGAAGTACAAGCTGAAG |
| | | CACCTGGTCTGGGCTTCTAGAGAACTGGAAAGATTCGCCCTGAATCCAGGTCTGCTGGAAACAGCAGCAGCA |
| | | GTGAAAGCAGCTTGTTGGTGGGCAGGAGTGAAACAGGAGTTCGGAATCCCCTACAACACACAGTCTCAGGGA |
| | | GTGGTGGAATCTATGAACAACGAGCTGAAGAAGATCATCGGCCAGATCAGAGACCAGGCCGAACATCTGAAG |
| | | ACAGCAGTGCAGATGGCAGTGCTGATTCACAATTTCAAGAGAAAGGGCGGCATCGGAGAGTATAGCGCCGGA |
| | | GAGAGAATCATCGATATCATCGCTACACAGGGCTTCTTCCCCGATTGGCAGAATTACACCCCAGGCCCAGGC |
| | | ATTAGATTCCCTCTGACATTCGGTTGGTGCTTCAAACTGGTGCCTCTGCTGATCAAGAAGGAGAAGATCTAC |
| | | CTGGCTTGGGTGCCAGCTCATAAAGGAATCGGAGGAAACGAGCAGATCGATAAGCTGGTGTCT |
| 134 | Pol:56-117 | TTTCCTCAGATCACTCTCTGGCAGAGACCACTGGTGACAATCAAGATCGGAGGACAGCTGAAAGAAGCTCTG |
| | Nef:64-99 | CTGGATACAGGAGCAGACGATACAGTGCTGGAAGAGATGAATCTGCCAGGTCGCTGGAAACCTAAGATGATT |
| | LI | GGAGGCATTGGCGGCTTTATCAAGGTGAGACAGTACGACCAGGAGGAAGTGGGATTTCCAGTGAAACCTCAG |
| | Pol:542-606 | GTGCCTCTGAGACCTATGACATTTAAGGGCGCTCTGGACCTGTCTCACTTTCTGAGAGAGAAGGGAGGACTG |
| | Pol:367-431 | GAAGGACTGATCCCTAAGTTCAAGCTGCCTATCCAGAAGGAGACTTGGGAAACTTGGTGGACAGAGTATTGG |
| | Pol:932-1003 | CAGGCTACTTGGATTCCCGAGTGGGAATTTGTGAACACACCTCCTCTGGTGAAGCTGTGGTATCAGCTGGAA |
| | Pol:129-320 | AAGGAGCCTATTGTGGGCGCAGAAACATTCTACGTGGACGGAGCAGCTAACAGAGAAACTAAGTGGGGATTC |
| | | ACCACCCCAGATAAGAAGCACCAGAAGGAGCCACCATTTCTCTGGATGGGATACGAACTGCACCCAGATAAG |
| | | TGGACAGTCCAGCCTATTGTGCTGCCAGAAAAGGACTCTTGGACCGTGAACGATATCCAGAAGCTGGTGGGA |
| | | AAGCTGAATTGGGCTTCTCAGATCTACCCAGGAATCAAGGTGATCACCAAGATCCAGAACTTCAGGGTGTAC |
| | | TACAGAGACAGCAGAGATCCTCTCTGGAAGGGACCAGCTAAACTCCTCTGGAAAGGAGAAGGAGCAGTGGTG |
| | | ATCCAGGATAACAGCGACATCAAGGTGGTGCCTAGAAGAAAGGCCAAGATCATCAGGGACTACGGCAAACAG |
| | | ATGGCAGGAGACGATTGCGTGGCTTCTAGACAGGACGAAGACGGAACAGTCCTGGTGGGACCTACACCAGTG |
| | | AATATCATCGGCAGAAATCTCCTGACACAGATCGGTTGTACCCTGAACTTCCCTATCAGCCCTATCGAGACA |
| | | GTGCCAGTGAAACTGAAGCCAGGAATGGACGGACCTAAAGTCAAGCAGTGGCCTCTGACAGAAGAGAAGATC |
| | | AAGGCCCTGGTGGAGATTTGCACAGAGATGGAGAAGGAGGGAAAGATCAGCAAGATCGGCCCAGAGAATCCT |
| | | TACAACACCCCAGTGTTCGCCATCAAGAAGAAGGATAGCACCAAGTGGAGAAAGCTGGTGGATTTCAGGGAG |
| | | CTGAACAAGAGAACCCAGGACTTTTGGGAAGTGCAGCTGGGCATCCCCCATCCAGCAGGACTGAAGAAGAAG |
| | | AAGAGCGTGACAGTGCTGGACGTGGGAGACGCTTATTTTAGCGTGCCTCTGGACAAGGACTTCAGAAAGTAC |
| | | ACCGCCTTCACCATCCCTTCTATCAACAACGAGACCCCAGGCATCAGATACCAGTATAACGTGCTGCCTCAG |
| | | GGTTGGAAAGGATCTCCAGCAATCTTTCAGTCTAGCATGACC |
| 135 | Pol:56-117 | TTCCCTCAGATTACTCTCTGGCAGAGGCCACTGGTGACAATTAAGATCGGAGGACAGCTGAAAGAAGCTCTG |
| | Nef:64-99 | CTGGATACAGGAGCAGACGATACAGTGCTGGAGGAAATGAACCTGCCAGGTCGCTGGAAACCTAAAATGATC |
| | LI | GGAGGAATCGGCGGCTTTATTAAGGTGAGACAGTACGATCAGGAGGAAGTGGGATTTCCAGTGAAACCTCAG |
| | Pol:542-606 | GTGCCTCTGAGACCTATGACATTTAAGGGCGCTCTGGATCTGAGCCACTTTCTGAGAGAGAAAGGAGGACTG |
| | Pol:367-431 | GAAGGACTGATCCCTAAGTTCAAGCTGCCCATCCAGAAGGAGACTTGGGAAACTTGGTGGACCGAGTATTGG |
| | Pol:932-1003 | CAGGCAACTTGGATTCCCGAGTGGGAATTTGTGAACACACCTCCTCTGGTGAAGCTGTGGTATCAGCTGGAA |
| | Pol:129-320 | AAGGAGCCTATCGTGGGAGCCGAAACATTTTACGTGGACGGAGCAGCCAACAGAGAAACTAAGTGGGGATTC |
| | | ACCACCCCAGATAAGAAGCACCAGAAAGAGCCTCCCTTTCTCTGGATGGGATACGAACTGCACCCAGATAAG |
| | | TGGACAGTCCAGCCTATTGTGCTGCCAGAAAAGGACTCTTGGACAGTGAACGACATCCAGAAGCTGGTGGGA |
| | | AAGCTGAATTGGGCCTCTCAGATTTACCCAGGAATCAAGGTGATCACCAAGATCCAGAACTTCAGGGTGTAC |
| | | TACAGGGATAGCAGAGATCCTCTCTGGAAGGGACCAGCTAAACTCCTCTGGAAAGGAGAAGGAGCAGTGGTG |
| | | ATCCAGGATAATAGCGACATCAAGGTGGTGCCTAGAAGAAAGGCTAAGATCATCCGGGACTACGGCAAACAG |
| | | ATGGCAGGAGACGATTGCGTGGCTTCTAGACAGGATGAAGACGGAACAGTCCTGGTGGGACCTACACCAGTG |
| | | AACATCATCGGCAGAAACCTGCTGACACAGATCGGTTGTACCCTGAACTTCCCTATCTCTCCTATCGAAACA |
| | | GTGCCAGTGAAGCTGAAGCCAGGAATGGACGGACCTAAAGTCAAGCAGTGGCCTCTGACAGAAGAGAAGATC |
| | | AAGGCTCTGGTGGAGATTTGCACCGAAATGGAGAAGGAGGGCAAGATCAGCAAGATCGGACCAGAGAATCCT |
| | | TACAATACCCCAGTGTTCGCCATCAAGAAGAAGGACAGCACCAAGTGGAGAAAGCTGGTGGATTTCAGGGAA |
| | | CTGAACAAGAGGACCCAGGACTTTTGGGAAGTGCAGCTGGGCATCCCCCATCCAGCAGGACTGAAGAAGAAG |
| | | AAGAGCGTGACAGTGCTGGACGTGGGAGACGCTTATTTTAGCGTGCCTCTGGACAAGGACTTCAGAAAGTAC |
| | | ACCGCCTTCACCATCCCTAGCATCAATAACGAGACCCCAGGCATCAGATACCAGTATAACGTGCTGCCACAG |
| | | GGCTGGAAAGGATCTCCAGCAATCTTTCAGAGCTCTATGACA |
| 136 | Pol:56-117 | CTGCCTCAGATTACCCTGTGGCAGAGACCTATCGTGACCATCAAGATCGGAGGACAGATCAAAGAAGCTCTG |
| | Pol:367-431 | CTGGATACAGGAGCAGACGATACAGTGCTGGAGGATATGAACCTGCCAGGTAAGTGGAAGCCTAAGATGATC |
| | AA | GGCGGAATTGGCGGCTTTATCAAGGTCAAGCAGTACGATCAGTGGGGACTGACAACACCAGACAAGAAGCAC |
| | QEE | CAGAAGGACCCCCCTTTCCTCTGGATGGGATACGAACTGCATCCAGATAGGTGGACAGTGCAGCCAATTGAG |
| | Nef:64-99 | CTGCCAGAAAAGGAGTCTTGGACAGTGAACGACATCCAGAAGCTGATCGGCAAGCTGAATTGGGCTTCTCAG |
| | Pol:932-1003 | ATCTACGCCGGAATTAAGGTGGCAGCTCAGGAAGAAGAAGAAGTGGGATTTCCAGTGAGACCTCAGGTGCCT |
| | Pol:542-606 | CTGAGACCTATGACATACAAGGGAGCTCTGGATCTGAGCCACTTTCTGAAAGAGAAGGGAGGACTGGAGGGA |
| | AA | ATTACCAAGCTGCAGAACTTCAGGGTGTACTACAGGGACAACAGAGATCCTCTGTGGAAAGGACCAGCTAGA |
| | Pol:129-320 | CTCCTCTGGAAAGGAGAAGGAGCAGTGGTGATTCAGGACAATAGCGAGATCAAGGTGGTGCCTAGAAGAAAG |
| | | GTGAAGATCATCCGGGACTACGGCAAAAGAATGGCAGGAGACGATTGCGTGGCAGGAAGACAGGACGAGGAC |
| | | CCCAAATTCAGACTGCCTATCCAGAAGGAGACTTGGGACACTTGGTGGACAGATTATTGGCAGGCAACTTGG |
| | | ATTCCCGAGTGGGAATTTACCAATACCCCTCCTCTGGTCAAGCTCTGGTATCAGCTGGAAACAGAGCCTATC |
| | | GCAGGAGTGGAAACATTCTACGTGGACGGAGCCTCTAATAGAGAGACAAAAGCCGCAGGAACAGTGCTGATT |
| | | GGACCTACACCAGTGAACATCATCGGGAGAAACCTGCTGACACAGCTGGGTTGTACACTGAACTTCCCTATC |
| | | AGCCCTATCGATACAGTGCCAGTGAAACTGAAGCCAGGAATGGACGGACCTAGAGTGAAACAGTGGCCTCTG |
| | | ACAGAAGAGAAGATCAAGGCCCTGATCGAGATTTGTACAGAGATGGAGAAGGAGGGCAAGATCTCTAGAATT |
| | | GGCCCAGAGAACCCCTACAATACCCCTATCTTTGCCATCAAGAAGAAGGACGGCACCAAGTGGAGAAAGCTG |
| | | GTGGATTTCAGGGAGCTGAACAAGAAGACCCAGGACTTTTGGGAAGTGCAGCTGGGCATCCCCCACCCTAGC |
| | | GGACTGAAAAAGAAGAAGAGCGTGACCGTGCTGGATATTGGAGACGCCTATTTTAGCGTGCCACTGGATAAG |
| | | GAGTTCAGAAAGTACACCGCCTTTACCGTGCCTTCTACCAATAACGAGACACCAGGAGTGAGATACCAGTAC |
| | | AACGTGCTGCCTATGGGTTGGAAGGGATCACCAGCCATCTTTCAGTGTAGCATGACA |
| 137 | Pol:56-117 | CTGCCTCAGATTACTCTCTGGCAGAGGCCTATTGTGACAATCAAGATCGGCGGACAGATCAAAGAAGCCCTG |
| | Pol:367-431 | CTGGATACAGGAGCAGACGATACAGTGCTGGAGGATATGAACCTGCCAGGCAAGTGGAAACCTAAGATGATC |
| | AA | GGAGGAATCGGCGGATTTATCAAGGTGAAGCAGTACGATCAGTGGGGACTGACAACACCAGATAAGAAGCAC |
| | QEE | CAGAAGGACCCCCCATTCCTGTGGATGGGATACGAACTGCATCCAGATAGGTGGACAGTGCAGCCAATCGAA |
| | Nef:64-99 | CTGCCAGAAAAGGAGTCTTGGACCGTGAACGACATCCAGAAACTGATCGGCAAGCTGAATTGGGCCAGCCAG |
| | Pol:932-1003 | ATTTACGCCGGAATCAAAGTGGCAGCTCAGGAAGAAGAGGAAGTGGGATTTCCAGTGAGACCTCAGGTGCCT |
| | Pol:542-606 | CTGAGACCTATGACATACAAAGGCGCTCTGGATCTGAGCCACTTTCTGAAAGAGAAGGGAGGACTGGAGGGA |
| | AA | ATTACAAAGCTGCAGAACTTCCGGGTGTACTACAGAGACAACAGAGACCCTCTCTGGAAAGGACCAGCTAGA |
| | Pol:129-320 | CTCCTCTGGAAAGGAGAAGGAGCAGTGGTGATCCAGGATAATAGCGAGATCAAGGTGGTGCCTAGGAGAAAG |
| | | GTGAAGATCATCAGGGATTACGGCAAAAGAATGGCCGGAGACGATTGCGTGGCAGGAAGACAGGACGAAGAT |
| | | CCCAAGTTCAGACTGCCTATCCAGAAGGAGACTTGGGACACTTGGTGGACCGATTATTGGCAGGCAACTTGG |
| | | ATTCCCGAGTGGGAATTTACCAACACACCTCCTCTGGTGAAGCTGTGGTATCAGCTGGAAACAGAGCCTATT |
| | | GCCGGAGTGGAAACATTCTACGTGGACGGAGCCAGCAACAGAGAGACAAAAGCCGCCGGAACAGTGCTGATT |
| | | GGACCTACACCCGTGAATATCATCGGAAGAAATCTGCTGACACAGCTGGGTTGTACCCTGAATTTCCCTATC |
| | | AGCCCCATCGATACAGTGCCAGTGAAACTGAAGCCAGGAATGGACGGACCTAGAGTCAAACAGTGGCCTCTG |
| | | ACAGAAGAGAAGATCAAGGCCCTGATCGAGATTTGTACCGAGATGGAGAAGGAGGGAAAGATCAGCAGAATC |
| | | GGCCCAGAGAATCCTTACAACACCCCCATCTTCGCCATCAAGAAGAAAGACGGAACCAAGTGGAGAAAGCTG |
| | | GTGGATTTCAGGGAGCTGAACAAGAAGACCCAGGACTTTTGGGAAGTGCAGCTGGGCATCCCCCACCCTAGC |
| | | GGCCTGAAGAAGAAAAAGAGCGTGACAGTGCTGGACATTGGAGACGCTTATTTCAGCGTGCCACTGGATAAG |
| | | GAGTTCAGAAAGTACACCGCCTTTACCGTGCCTTCTACAAACAACGAGACACCAGGCGTGAGATATCAGTAC |
| | | AACGTGCTGCCTATGGGTTGGAAAGGATCTCCCGCCATCTTTCAGTGTAGCATGACA |
| 138 | Pol:747-827 | GTGGCCAAAGAAATTGTGGCCTCTTGCGATAAGTGCCAGCTGAAAGGAGAGGCTATGCACGGACAGGTGGAT |
| | Nef:117-148 | TGTTCTCCAGGAATTTGGCAGCTGGATTGTACACACCTGGAGGGAAAGATTATTCTGGTGGCAGTGCACGTG |
| | Pol:840-920 | GCATCAGGATATATTGAGGCCGAAGTGATTCCAGCAGAAACAGGACAGGAGACAGCTTACTTTCTGCTCAAA |
| | AA | CTGGCAGGTCGCTGGCCAGTGAAGACAACACAGGGCTACTTTCCTGATTGGCAGAATTACACACCAGGACCA |
| | Gag:1-53 | GGAACAAGATACCCTCTGACCTTTGGTTGGTGCTTCAAACTGGTGCCCGTGACAGTGAAAGCAGCTTGTTGG |
| | SEG | TGGGCAGGAATTAAGCAGGAGTTCGGCATCCCTTACAATCCTCAGTCTCAGGGAGTGGTGGAATCTATGAAC |
| | Gag:147-369 | AAGGAGCTGAAGAAGATCATCGGCCAGGTGAGAGATCAGGCAGAACATCTGAAGACAGCAGTGCAGATGGCA |
| | AAA | GTGTTCATCCACAACTTCAAGCGGAAGGGAGGAATTGGAGGATATAGCGCAGGAGAGAGAATCGTGGATATC |
| | Pol:683-708 | ATTGCCGCCGCTATGGCAGCTAGAGCCAGCGTGCTGAGCGGAGGAGAACTCGATCGCTGGGAAAAGATCAGA |
| | | CTGAGACCAGGAGGCAAGAAGAAGTACAGACTGAAGCACATCGTCTGGGCTTCTAGAGAACTGGAGAGATTT |
| | | GCCGTGAATCCAGGACTGCTGGAAACAAGCGAGGGCATTTCTCCTAGAACCCTGAACGCTTGGGTGAAAGTG |
| | | GTGGAAGAAAAAGCCTTCTCTCCAGAGGTGATCCCTATGTTTAGCGCTCTGTCAGAAGGAGCTACACCTCAG |
| | | GATCTGAACACCATGCTGAACACAGTGGGAGGACATCAGGCAGCTATGCAGATGCTGAAGGAGACAATTAAC |
| | | GAAGAAGCCGCCGAGTGGGATAGACTGCATCCAGTGCACGCAGGACCTATTGCTCCAGGACAGATGAGAGAG |
| | | CCTAGAGGAAGCGATATTGCCGGAACAACAAGCACACTGCAGGAACAGATCGGTTGGATGACCAATAATCCC |
| | | CCTATTCCAGTGGGCGAGATCTATAAGCGCTGGATTATCCTGGGCCTGAACAAGATCGTGAGAATGTACAGC |
| | | CCCACCTCTATCCTGGATATCAGACAGGGCCCTAAGGAACCTTTCAGAGACTACGTGGACAGGTTCTACAAG |
| | | ACACTGAGAGCAGAACAGGCATCTCAGGAGGTGAAGAATTGGATGACCGAGACACTGCTGGTGCAGAACGCC |
| | | AATCCAGATTGCAAGACAATTCTGAAAGCCCTGGGACCAGCAGCTACACTGGAAGAGATGATGACCGCTTGT |
| | | CAGGGAGTGGGAGGACCAGGACATAAAGCTAGAGTGCTGGCAGAAGCTATGTCTCAGGCAGCAGCTAAGGAG |
| | | AAAGTGTATCTGGCTTGGGTGCCAGCCCATAAAGGAATTGGAGGAAACGAGCAGGTGGATAAACTGGTGTCT |
| 139 | Pol:747-827 | GTGGCTAAGGAAATTGTGGCCTCTTGCGACAAGTGTCAGCTGAAAGGAGAGGCTATGCACGGACAGGTGGAT |
| | Nef:117-148 | TGTTCTCCAGGAATTTGGCAGCTGGATTGCACACATCTGGAAGGAAAGATTATTCTGGTGGCAGTGCACGTG |
| | Pol:840-920 | GCATCTGGATATATCGAGGCCGAGGTGATTCCAGCCGAAACAGGACAGGAAACAGCCTACTTTCTCCTGAAA |
| | AA | CTGGCAGGTAGGTGGCCAGTGAAGACAACACAGGGCTACTTCCCAGATTGGCAGAATTACACCCCAGGACCA |
| | Gag:1-53 | GGAACAAGATACCCTCTGACCTTTGGTTGGTGCTTCAAGCTCGTCCCAGTGACAGTGAAAGCAGCTTGTTGG |
| | SEG | TGGGCAGGAATTAAACAGGAGTTCGGAATCCCTTACAATCCTCAGTCTCAGGGAGTGGTGGAAAGCATGAAC |
| | Gag:147-369 | AAGGAGCTGAAGAAGATCATCGGACAGGTGAGAGATCAGGCAGAACATCTGAAGACAGCAGTGCAGATGGCA |
| | AAA | GTGTTCATCCACAACTTCAAGAGGAAGGGCGGAATTGGAGGATATAGCGCCGGAGAGAGAATCGTGGATATC |
| | Pol:683-708 | ATTGCAGCAGCTATGGCAGCTAGAGCTTCAGTGCTGTCAGGAGGAGAACTCGATAGGTGGGAGAAGATCAGA |
| | | CTGAGACCAGGAGGCAAGAAGAAGTACAGACTGAAGCACATCGTGTGGGCTTCTAGAGAACTGGAGAGATTC |
| | | GCAGTGAATCCAGGACTGCTGGAAACAAGCGAGGGAATTAGCCCTAGAACCCTGAATGCTTGGGTGAAAGTG |
| | | GTGGAAGAGAAGGCCTTCAGCCCAGAGGTGATCCCTATGTTTAGCGCTCTGTCAGAAGGAGCTACACCTCAG |
| | | GATCTGAACACCATGCTGAATACAGTGGGAGGACATCAGGCAGCTATGCAGATGCTGAAGGAGACCATCAAC |
| | | GAAGAAGCAGCCGAGTGGGATAGACTGCATCCAGTGCACGCAGGACCTATTGCTCCAGGACAGATGAGAGAA |
| | | CCTAGAGGAAGCGATATCGCCGGAACAACATCTACACTGCAGGAACAGATCGGTTGGATGACCAACAACCCT |
| | | CCTATTCCAGTGGGCGAGATTTACAAGCGCTGGATTATCCTGGGCCTGAATAAGATCGTGAGAATGTACAGC |
| | | CCTACCAGCATTCTGGACATCAGACAGGGACCTAAGGAGCCTTTTAGAGACTACGTGGACAGGTTCTACAAG |
| | | ACCCTGAGAGCAGAACAGGCATCTCAGGAGGTGAAGAATTGGATGACCGAGACACTGCTGGTGCAGAACGCT |
| | | AATCCCGATTGCAAGACCATCCTGAAAGCTCTGGGACCAGCAGCTACACTGGAAGAGATGATGACAGCTTGT |
| | | CAGGGAGTGGGAGGACCAGGACATAAAGCTAGAGTGCTGGCAGAAGCTATGTCTCAGGCAGCAGCTAAAGAG |
| | | AAAGTGTATCTGGCTTGGGTGCCAGCTCACAAAGGAATTGGAGGAAACGAGCAGGTGGATAAACTGGTGAGC |
| 140 | Pol:747-827 | GTGGCCAAAGAGATTGTGGCCTCCTGTGACAAGTGCCAGCTGAAAGGAGAGGCAATGCATGGACAGGTGGAT |
| | Nef:117-148 | TGTTCTCCAGGAATCTGGCAGCTGGATTGCACACACCTGGAGGGAAAGATCATCCTGGTGGCAGTGCATGTT |
| | Pol:840-920 | GCATCAGGATACATTGAGGCAGAAGTGATTCCAGCAGAAACAGGACAGGAGACTGCTTACTTTCTGCTGAAA |
| | AA | CTGGCAGGAAGGTGGCCAGTGAAGACAACACAGGGTTATTTCCCTGATTGGCAGAACTACACCCCAGGCCCT |
| | Gag:1-53 | GGCACAAGATACCCTCTGACCTTTGGTTGGTGCTTCAAACTGGTCCCTGTGACAGTGAAAGCAGCTTGTTGG |
| | SEG | TGGGCAGGCATCAAGCAGGAGTTTGGCATCCCTTACAACCCTCAGTCTCAGGGAGTTGTGGAATCCATGAAC |
| | Gag:147-369 | AAGGAGCTGAAGAAGATCATTGGTCAGGTGAGGGATCAGGCAGAACATCTGAAGACAGCAGTGCAGATGGCA |
| | AAA | GTGTTCATCCACAATTTCAAGAGGAAGGGAGGAATTGGAGGATACAGTGCAGGAGAGAGAATTGTGGACATC |
| | Pol:683-708 | ATTGCAGCTGCAATGGCAGCAAGAGCCAGTGTGCTCAGTGGAGGAGAACTTGACAGGTGGGAAAAGATCAGA |
| | | CTGAGACCAGGAGGCAAGAAGAAGTACAGACTGAAGCACATTGTCTGGGCTTCCAGAGAACTGGAGAGATTT |
| | | GCTGTGAATCCAGGGCTGCTGGAAACAAGTGAGGGCATTTCTCCCAGAACTCTGAATGCTTGGGTGAAGGTG |
| | | GTGGAAGAAAAAGCCTTCTCTCCAGAGGTGATCCCCATGTTCAGTGCACTGTCTGAAGGAGCCACACCTCAG |
| | | GACCTCAACACCATGCTGAACACAGTGGGAGGACATCAGGCAGCCATGCAGATGCTGAAGGAGACCATCAAT |
| | | GAAGAAGCTGCAGAGTGGGACAGGCTGCATCCAGTTCATGCAGGACCAATTGCTCCTGGACAGATGAGAGAG |
| | | CCCAGAGGAAGTGACATTGCTGGCACAACCAGCACACTGCAGGAACAGATTGGTTGGATGACCAACAATCCC |
| | | CCCATTCCAGTGGGAGAGATCTACAAGAGGTGGATCATCCTTGGCCTGAACAAGATTGTGAGAATGTACAGC |
| | | CCCACTTCAATCCTGGACATCAGACAGGGCCCCAAGGAACCTTTCAGAGACTATGTGGACAGGTTCTACAAG |
| | | ACACTGAGAGCAGAACAGGCCTCACAGGAGGTGAAGAATTGGATGACTGAGACACTGCTGGTGCAGAATGCC |
| | | AATCCAGATTGCAAGACAATTCTGAAAGCCCTGGGTCCAGCAGCCACACTGGAAGAGATGATGACAGCTTGC |
| | | CAGGGAGTGGGTGGACCAGGACACAAAGCAAGAGTGCTGGCAGAAGCAATGTCTCAGGCTGCAGCCAAGGAG |
| | | AAAGTTTATCTGGCTTGGGTCCCAGCGCACAAAGGAATTGGAGGAAATGAGCAGGTGGACAAACTTGTGTCC |
| 141 | Gag:147-369 | CTGTCTCCTAGAACACTGAACGCTTGGGTGAAAGTGATCGAGGAAAAGGCCTTTAGCCCAGAAGTGATCCCT |
| | Pol:840-920 | ATGTTTACCGCCCTGTCAGAAGGAGCTACACCTCACGATCTGAACACCATGCTGAACACAATCGGAGGACAT |
| | Pol:683-708 | CAGGCAGCTATGCAGATGCTGAAGGATACAATCAACGAAGAAGCCGCCGAGTGGGATAGAGTGCATCCAGTG |
| | AAY | CACGCAGGACCAGTGGCTCCAGGACAGATGAGAGATCCTAGAGGAAGCGATATCGCAGGATCTACAAGCACA |
| | Gag:1-53 | CTGCAGGAACAGATCGCTTGGATGACCAATAATCCCCCTATTCCAGTGGGCGATATCTACAAGCGCTGGATC |
| | Nef:117-148 | ATCATGGGCCTGAACAAGATCGTGAGGATGTACAGCCCAGTGAGCATCCTGGATATCAAGCAGGGACCTAAG |
| | Pol:747-827 | GAGCCTTTCAGAGATTACGTGGACAGGTTCTACAGAACACTGAGAGCCGAACAGGCATCTCAGGACGTGAAG |
| | | AATTGGATGACCGAGACACTGCTGGTGCAGAACAGCAATCCCGATTGCAAGACAATCCTGAAAGCTCTGGGA |
| | | CCAGGAGCTACACTGGAGGAAATGATGAGCGCTTGTCAGGGAGTGGGAGGACCATCTCATAAAGCTAGAGTG |
| | | CTGGCCGAAGCTATGTGTCAGGCAGTGAAAGCAGCTTGTTGGTGGGCAGGAGTGAAACAGGAGTTCGGCATC |
| | | CCTTACAACACCCAGTCTCAGGGAGTGGTGGAATCTATGAACAACGAGCTGAAGAAGATCATCGGCCAGATC |
| | | AGAGACCAGGCAGAACATCTGAAGACAGCAGTGCAGATGGCAGTGCTGATTCACAACTTCAAGAGAAAGGGC |
| | | GGCATTGGAGAGTATAGCGCCGGAGAGAGAATTATCGATATCATCGCCAAGGAGAAGATCTATCTGGCTTGG |
| | | GTGCCAGCTCATAAAGGAATCGGAGGAAACGAGCAGATCGATAAGCTGGTGTCAGCCGCCTATATGGCAGCT |
| | | AGAGCTTCTATTCTGAGCGGAGGAAAGCTCGACAAGTGGGAAAAGATCAGGCTGAGACCAGGAGGCAGAAAG |
| | | AAGTACAAGCTGAAGCATCTCGTCTGGGCTTCTAGAGAACTGGAAAGATTCGCTCTGAATCCAGGACTGCTG |
| | | GAAACAACCCAGGGCTTCTTCCCCGATTGGCAGAATTACACCCCAGGACCAGGAATCAGATTCCCTCTGACC |
| | | TTCGGTTGGTGTTTCAAGCTGGTGCCTCTGGTGGCTAAAGAGATTGTGGCTTGTTGCGACAAGTGTCAGCTG |
| | | AAAGGAGAGGCTATTCACGGACAGGTGGATTGTTCTCCAGGAGTCTGGCAGCTGGATTGTACACATCTGGAG |
| | | GGAAAAGTGATTCTGGTGGCAGTGCACGTGGCATCAGGATATATTGAGGCCGAAATCATCCCTACAGAGACA |
| | | GGACAGGAGACAGCCTACTTTATCCTGAAGCTGGCAGGAAGATGGCCAGTGACAACA |
| 142 | Gag:147-369 | CTGTCTCCTAGAACACTGAACGCTTGGGTGAAGGTGATTGAGGAGAAAGCCTTCAGCCCAGAAGTGATCCCT |
| | Pol:840-920 | ATGTTTACAGCCCTGAGCGAAGGAGCTACACCTCACGATCTGAATACCATGCTGAATACAATCGGCGGACAT |
| | Pol:683-708 | CAGGCAGCCATGCAGATGCTGAAGGATACAATCAACGAAGAAGCAGCCGAGTGGGATAGAGTGCATCCAGTG |
| | AAY | CACGCAGGACCAGTGGCTCCAGGACAGATGAGAGATCCTAGAGGAAGCGATATCGCCGGATCTACATCTACA |
| | Gag:1-53 | CTGCAGGAACAGATCGCTTGGATGACAAATAACCCCCCTATCCCAGTGGGAGATATCTATAAGCGCTGGATC |
| | Nef:117-148 | ATCATGGGCCTGAACAAGATCGTGAGGATGTACAGCCCAGTGAGCATCCTGGATATCAAGCAGGGACCTAAG |
| | Pol:747-827 | GAGCCTTTCAGAGATTACGTGGACAGGTTCTACAGAACCCTGAGAGCAGAACAGGCTTCTCAGGACGTGAAG |
| | | AATTGGATGACCGAAACACTGCTGGTGCAGAATAGCAACCCCGATTGCAAGACCATCCTGAAAGCTCTGGGA |
| | | CCAGGAGCTACACTGGAAGAAATGATGAGCGCTTGTCAGGGAGTGGGAGGACCATCTCATAAGGCTAGAGTG |
| | | CTGGCAGAAGCTATGTGTCAGGCAGTGAAAGCAGCTTGTTGGTGGGCAGGAGTGAAACAGGAGTTTGGCATC |
| | | CCTTACAATACCCAGTCTCAGGGAGTGGTGGAAAGCATGAACAACGAGCTGAAGAAGATCATCGGCCAGATT |
| | | AGAGATCAGGCCGAACATCTGAAAACAGCAGTGCAGATGGCCGTGCTGATCCACAATTTCAAGAGGAAGGGC |
| | | GGAATTGGCGAATATAGCGCCGGAGAGAGAATCATCGACATCATCGCCAAGGAGAAGATCTACCTGGCTTGG |
| | | GTGCCAGCTCATAAAGGAATCGGAGGAAACGAGCAGATCGATAAACTGGTGTCTGCCGCTTATATGGCAGCT |
| | | AGAGCTTCTATCCTGTCAGGAGGAAAGCTCGATAAGTGGGAAAAGATCAGACTGAGACCAGGCGGAAGAAAG |
| | | AAGTACAAGCTGAAGCACCTCGTCTGGGCTTCTAGAGAACTGGAAAGATTCGCCCTGAATCCAGGACTGCTG |
| | | GAAACAACACAGGGATTCTTCCCCGATTGGCAGAACTACACACCAGGACCAGGCATCAGATTTCCTCTGACC |
| | | TTTGGTTGGTGCTTTAAACTGGTGCCTCTGGTGGCTAAGGAGATTGTGGCTTGTTGCGACAAGTGTCAGCTG |
| | | AAAGGAGAGGCTATTCACGGACAGGTGGATTGTTCTCCAGGAGTCTGGCAGCTGGATTGTACACACCTGGAG |
| | | GGAAAAGTGATTCTGGTGGCAGTGCACGTGGCATCAGGATATATCGAGGCCGAGATCATCCCTACAGAGACA |
| | | GGACAGGAAACCGCCTACTTTATCCTGAAACTGGCAGGTAGGTGGCCAGTGACAACA |
| 143 | Gag:147-369 | CTGAGCCCCAGAACCCTCAATGCCTGGGTCAAAGTGATTGAGGAAAAGGCCTTCAGCCCAGAGGTGATCCCC |
| | Pol:840-920 | ATGTTCACAGCCCTCAGTGAGGGGGCCACCCCCCATGACCTGAACACCATGCTCAACACCATTGGGGGCCAC |
| | Pol:683-708 | CAGGCTGCCATGCAGATGCTGAAGGACACCATCAATGAGGAAGCTGCTGAGTGGGACAGAGTCCACCCAGTG |
| | AAY | CATGCTGGCCCAGTGGCCCCAGGCCAGATGAGGGACCCCAGGGGCTCTGACATTGCTGGCAGCACCAGCACC |
| | Gag:1-53 | CTGCAGGAGCAGATAGCCTGGATGACCAACAACCCCCCCATCCCTGTGGGAGACATCTACAAAAGATGGATC |
| | Nef:117-148 | ATCATGGGCCTCAACAAGATTGTCAGGATGTACTCCCCTGTGTCCATCCTGGACATCAAGCAGGGCCCCAAG |
| | Pol:747-827 | GAACCCTTCAGGGACTATGTGGACAGATTCTACAGAACCCTGAGAGCTGAGCAGGCCTCCCAGGATGTGAAA |
| | | AACTGGATGACAGAGACCCTCCTGGTCCAGAACAGCAACCCAGACTGCAAGACCATCCTCAAGGCCTTGGGC |
| | | CCAGGAGCCACCCTGGAAGAGATGATGAGTGCCTGCCAGGGAGTTGGAGGCCCCAGCCACAAGGCCAGAGTG |
| | | CTGGCAGAGGCCATGTGCCAGGCTGTGAAGGCTGCCTGCTGGTGGGCAGGAGTCAAGCAGGAGTTTGGCATC |
| | | CCATACAACACCCAGAGCCAGGGTGTGGTGGAAAGCATGAACAATGAGCTGAAAAAGATCATTGGCCAGATC |
| | | AGAGACCAGGCTGAGCACCTGAAGACAGCAGTGCAGATGGCTGTGCTCATCCACAACTTCAAGAGGAAAGGT |
| | | GGCATAGGGGAATACAGTGCTGGGGAGAGGATCATTGACATCATTGCCAAGGAGAAGATCTACCTGGCCTGG |
| | | GTGCCTGCCCACAAGGGCATTGGTGGCAATGAGCAGATTGACAAGCTGGTCTCAGCAGCCTACATGGCTGCC |
| | | AGAGCCTCCATCCTCTCAGGGGGCAAGCTGGACAAGTGGGAGAAAATCAGACTGAGGCCTGGTGGCAGAAAG |
| | | AAGTACAAGCTGAAGCACCTGGTGTGGGCCTCCAGGGAACTGGAAAGATTTGCCCTGAACCCTGGCCTGCTG |
| | | GAAACCACCCAGGGCTTCTTCCCTGACTGGCAGAACTACACCCCAGGCCCAGGCATCAGGTTCCCCCTGACC |
| | | TTTGGCTGGTGCTTCAAGCTGGTGCCCCTGGTGGCCAAGGAAATAGTGGCCTGCTGTGACAAGTGCCAGCTG |
| | | AAAGGGGAGGCCATCCATGGCCAAGTGGACTGCAGCCCTGGTGTGTGGCAGCTGGACTGCACCCACCTGGAA |
| | | GGCAAGGTCATCCTGGTTGCAGTGCATGTGGCCAGTGGCTACATTGAGGCTGAGATCATCCCCACAGAGACA |
| | | GGCCAGGAGACTGCCTACTTCATCCTGAAACTTGCAGGCAGGTGGCCTGTGACCACC |
| 144 | Pol:932-1003 | ATCACCAAGATCCAGAACTTCAGGGTGTACTACAGAGACAGCAGAGATCCTCTCTGGAAGGGACCAGCTAAA |
| | AAY | CTCCTCTGGAAAGGAGAAGGAGCAGTGGTGATCCAGGATAACAGCGACATCAAGGTGGTGCCTAGAAGAAAG |
| | EE | GCCAAGATCATCAGGGACTACGGCAAACAGATGGCAGGAGACGATTGCGTGGCTTCTAGACAGGACGAAGAC |
| | Nef:64-99 | GCAGCTTACGAAGAGGAAGAGGTGGGATTTCCAGTGAAACCTCAGGTGCCTCTGAGACCTATGACATTCAAG |
| | AA | GGAGCTCTGGATCTGTCTCACTTCCTGAGAGAAAAGGGAGGACTGGAAGGAGCAGCTTGGGGATTTACCACC |
| | Pol:367-431 | CCAGACAAGAAGCACCAGAAGGAACCACCATTCCTCTGGATGGGATACGAACTGCACCCAGATAAGTGGACA |
| | Pol:542-606 | GTCCAGCCTATTGTGCTGCCAGAAAAGGACTCTTGGACCGTGAACGATATCCAGAAGCTGGTGGGAAAGCTG |
| | Pol:56-117 | AATTGGGCTTCTCAGATCTACCCAGGAATCAAGGTGCCCAAGTTCAAGCTGCCTATCCAGAAGGAGACTTGG |
| | Pol:129-320 | GAAACTTGGTGGACAGAGTATTGGCAGGCTACTTGGATTCCCGAGTGGGAATTTGTGAACACACCTCCTCTG |
| | | GTGAAGCTGTGGTATCAGCTGGAAAAGGAGCCTATCGTGGGCGCAGAAACATTTTACGTGGACGGAGCCGCC |
| | | AATAGAGAAACCAAGTTTCCTCAGATCACTCTCTGGCAGAGACCTCTGGTGACAATCAAGATCGGCGGACAG |
| | | CTGAAAGAGGCTCTGCTGGATACAGGAGCAGACGATACCGTGCTGGAAGAAATGAATCTGCCAGGTAGGTGG |
| | | AAGCCTAAGATGATTGGCGGAATTGGCGGCTTCATCAAGGTGAGACAGTACGATCAGGGAACAGTGCTGGTG |
| | | GGACCTACTCCAGTGAACATCATCGGAAGGAACCTGCTGACACAGATCGGCTGTACACTGAACTTCCCTATC |
| | | AGCCCTATCGAGACAGTGCCAGTGAAACTGAAGCCAGGAATGGACGGACCTAAAGTCAAACAGTGGCCTCTG |
| | | ACAGAGGAGAAAATCAAAGCCCTGGTGGAGATTTGTACCGAGATGGAGAAGGAGGGCAAGATTTCTAAGATC |
| | | GGACCAGAGAACCCCTACAATACCCCAGTGTTTGCCATCAAGAAGAAGGACAGCACCAAGTGGAGGAAGCTG |
| | | GTGGATTTTAGGGAGCTGAACAAGAGGACACAGGACTTTTGGGAAGTGCAGCTGGGCATCCCCCACCCAGCC |
| | | GGACTGAAAAAGAAGAAGTCAGTGACAGTGCTGGACGTGGGAGATGCCTATTTTAGCGTGCCTCTGGATAAG |
| | | GACTTCAGGAAGTACACCGCCTTCACAATCCCTAGCATCAACAACGAGACCCCAGGAATCAGATACCAGTAC |
| | | AACGTGCTGCCTCAGGGTTGGAAAGGAAGCCCAGCCATCTTTCAGTCTAGCATGACC |
| 145 | Pol:932-1003 | ATCACCAAGATCCAGAACTTCCGGGTGTACTACAGGGATAGCAGAGATCCTCTCTGGAAGGGACCAGCTAAA |
| | AAY | CTCCTCTGGAAAGGAGAAGGAGCAGTGGTGATCCAGGATAATAGCGACATCAAGGTGGTGCCTAGAAGAAAG |
| | EE | GCTAAGATCATCCGGGACTACGGCAAACAGATGGCAGGAGACGATTGCGTGGCTTCTAGACAGGATGAAGAC |
| | Nef:64-99 | GCAGCCTACGAAGAAGAGGAAGTGGGATTTCCAGTGAAACCTCAGGTGCCTCTGAGACCTATGACATTCAAG |
| | AA | GGAGCTCTGGACCTGTCTCACTTTCTGAGAGAGAAGGGAGGACTGGAAGGAGCAGCTTGGGGATTTACCACA |
| | Pol:367-431 | CCAGATAAGAAGCACCAGAAGGAGCCACCATTTCTCTGGATGGGATACGAACTGCACCCAGATAAGTGGACA |
| | Pol:542-606 | GTCCAGCCTATTGTGCTGCCAGAAAAGGACTCTTGGACAGTGAACGACATCCAGAAGCTGGTGGGAAAGCTG |
| | Pol:56-117 | AATTGGGCCTCTCAGATTTACCCAGGAATCAAGGTGCCCAAGTTTAAGCTGCCTATCCAGAAGGAGACTTGG |
| | Pol:129-320 | GAAACTTGGTGGACCGAGTATTGGCAGGCAACTTGGATTCCCGAGTGGGAATTTGTGAACACACCTCCTCTG |
| | | GTGAAGCTGTGGTATCAGCTGGAAAAGGAGCCTATCGTGGGAGCCGAAACATTTTACGTGGACGGAGCAGCC |
| | | AACAGAGAGACAAAGTTCCCTCAGATCACTCTCTGGCAGAGACCACTGGTGACAATTAAGATCGGAGGACAG |
| | | CTGAAAGAAGCTCTGCTGGATACAGGAGCAGACGATACAGTGCTGGAGGAAATGAACCTGCCAGGTCGCTGG |
| | | AAACCTAAAATGATCGGCGGCATTGGCGGATTTATCAAGGTGAGGCAGTACGATCAGGGAACAGTGCTGGTG |
| | | GGACCTACACCCGTGAATATTATCGGAAGGAATCTGCTGACACAGATTGGCTGTACCCTGAACTTCCCTATC |
| | | AGCCCTATCGAAACCGTGCCAGTGAAACTGAAACCAGGAATGGACGGACCTAAAGTCAAGCAGTGGCCTCTG |
| | | ACAGAAGAGAAGATCAAAGCCCTGGTGGAGATTTGTACCGAGATGGAGAAGGAGGGAAAGATCAGCAAGATC |
| | | GGCCCAGAGAATCCTTACAACACCCCAGTGTTCGCCATCAAGAAGAAGGATAGCACCAAGTGGAGAAAGCTG |
| | | GTGGATTTCAGGGAGCTGAACAAGAGAACCCAGGACTTTTGGGAAGTGCAGCTGGGCATCCCCCACCCTGCC |
| | | GGCCTGAAGAAGAAGAAAAGCGTGACAGTGCTGGACGTGGGAGACGCTTATTTTAGCGTGCCTCTGGACAAG |
| | | GACTTCAGAAAGTACACCGCCTTCACCATCCCTTCTATCAATAACGAGACCCCAGGCATCAGATACCAGTAT |
| | | AACGTGCTGCCTCAGGGTTGGAAAGGAAGCCCAGCCATTTTTCAGAGCAGCATGACA |
| 146 | Pol:932-1003 | ATCACCAAGATCCAGAACTTCAGGGTCTACTACAGAGACAGCAGAGATCCACTCTGGAAGGGCCCAGCCAAA |
| | AAY | CTCCTTTGGAAAGGAGAAGGAGCAGTGGTGATCCAGGACAACAGTGACATCAAGGTGGTTCCCAGAAGAAAG |
| | EE | GCCAAGATCATCAGGGACTATGGCAAACAGATGGCAGGAGATGACTGTGTGGCTTCCAGACAGGATGAAGAT |
| | Nef:64-99 | GCAGCTTATGAAGAGGAAGAGGTGGGATTCCCAGTGAAACCCCAGGTGCCTCTGAGGCCAATGACATTCAAG |
| | AA | GGAGCTCTGGATCTGTCCCACTTCCTGAGAGAAAAGGGAGGACTGGAAGGAGCAGCTTGGGGATTCACCACC |
| | Pol:367-431 | CCAGACAAGAAGCATCAGAAGGAACCTCCCTTCCTCTGGATGGGTTATGAACTGCACCCAGACAAGTGGACA |
| | Pol:542-606 | GTCCAGCCCATTGTGCTGCCAGAAAAGGATTCTTGGACAGTGAATGACATCCAGAAGCTGGTGGGCAAGCTG |
| | Pol:56-117 | AATTGGGCCTCCCAGATCTACCCAGGAATCAAGGTGCCCAAGTTCAAGCTCCCCATCCAGAAGGAGACTTGG |
| | Pol:129-320 | GAAACTTGGTGGACAGAGTATTGGCAGGCAACTTGGATTCCTGAGTGGGAATTTGTGAACACCCCCCCTCTG |
| | | GTGAAGCTGTGGTATCAGCTGGAAAAGGAGCCCATTGTGGGGGCAGAAACATTTTATGTGGATGGAGCAGCC |
| | | AACAGAGAAACCAAGTTTCCCCAGATCACTCTTTGGCAGAGACCACTGGTGACAATCAAGATTGGGGGACAG |
| | | CTGAAAGAGGCTCTGCTGGACACAGGAGCTGATGACACAGTGCTGGAAGAAATGAATCTGCCAGGCAGGTGG |
| | | AAGCCCAAGATGATTGGAGGCATTGGGGGTTTCATCAAGGTGAGACAGTATGACCAGGGAACTGTGCTGGTG |
| | | GGACCCACTCCAGTGAACATCATTGGCAGGAACCTGCTGACACAGATTGGCTGCACACTGAACTTCCCCATC |
| | | AGCCCCATTGAGACAGTCCCAGTGAAACTGAAGCCAGGAATGGATGGCCCAAAAGTCAAACAATGGCCCCTG |
| | | ACAGAGGAGAAAATCAAAGCTCTGGTGGAGATTTGCACAGAGATGGAGAAGGAGGGCAAGATCTCTAAGATT |
| | | GGCCCAGAGAACCCATACAACACTCCAGTGTTTGCAATCAAGAAGAAGGACAGCACCAAGTGGAGGAAGCTG |
| | | GTGGACTTCAGGGAGCTCAACAAGAGGACACAGGACTTTTGGGAAGTGCAGCTGGGCATTCCCCACCCAGCA |
| | | GGACTGAAAAAGAAGAAGTCAGTGACAGTGCTGGATGTGGGGGATGCTTATTTCAGTGTGCCTCTGGACAAG |
| | | GACTTCAGGAAGTACACAGCCTTCACAATCCCCAGCATCAACAATGAGACCCCAGGAATCAGATATCAGTAC |
| | | AATGTGCTGCCTCAGGGTTGGAAAGGAAGCCCAGCCATTTTCCAGTCGAGCATGACC |
| 147 | Pol:542-606 | CCTAAATTCAGACTGCCTATCCAGAAGGAGACTTGGGATACTTGGTGGACCGATTATTGGCAGGCAACTTGG |
| | Nef:64-99 | ATTCCCGAGTGGGAATTTACCAATACCCCTCCTCTGGTGAAGCTGTGGTATCAGCTGGAAACAGAGCCTATT |
| | Pol:56-117 | GCCGGAGTGGAAACATTCTACGTGGACGGAGCCTCTAATAGAGAGACCAAGGAGGAAGTGGGATTTCCAGTG |
| | Pol:932-1003 | AGACCTCAGGTGCCTCTGAGACCTATGACATACAAGGGAGCTCTGGACCTGTCTCACTTTCTGAAGGAGAAG |
| | Pol:367-431 | GGAGGACTGGAAGGACTGCCTCAGATTACTCTCTGGCAGAGACCTATCGTGACCATCAAGATCGGAGGACAG |
| | K | ATCAAAGAAGCTCTGCTGGATACAGGAGCAGACGATACAGTGCTGGAGGATATGAACCTGCCAGGTAAGTGG |
| | Pol:129-320 | AAGCCTAAGATGATCGGCGGAATTGGCGGCTTTATCAAGGTCAAGCAGTACGACCAGATCACCAAGCTGCAG |
| | | AACTTCAGGGTGTACTACAGGGACAACAGAGACCCCCTCTGGAAAGGACCAGCTAGACTCCTCTGGAAAGGA |
| | | GAAGGAGCAGTGGTGATCCAGGACAATAGCGAGATCAAGGTGGTGCCTAGAAGGAAGGTGAAGATCATCAGG |
| | | GACTACGGAAAAAGAATGGCCGGAGACGATTGCGTGGCAGGAAGACAGGACGAAGATTGGGGACTGACAACA |
| | | CCAGACAAGAAGCACCAGAAGGACCCCCCATTCCTCTGGATGGGATACGAACTGCATCCAGATAGGTGGACA |
| | | GTGCAGCCAATTGAACTGCCAGAGAAGGAGTCTTGGACCGTGAACGATATCCAGAAGCTGATCGGCAAGCTG |
| | | AATTGGGCTTCTCAGATCTACGCAGGAATCAAGGTGAAGGGCACAGTGCTGATTGGACCTACACCAGTGAAC |
| | | ATCATCGGCAGAAACCTCCTGACACAGCTGGGTTGTACACTGAATTTCCCTATCAGCCCTATCGATACAGTG |
| | | CCAGTGAAGCTGAAACCAGGAATGGACGGACCTAGAGTCAAACAGTGGCCTCTGACAGAAGAGAAGATCAAG |
| | | GCCCTGATCGAGATTTGCACCGAGATGGAGAAGGAGGGAAAGATCTCTAGGATCGGCCCAGAGAATCCTTAC |
| | | AATACCCCTATCTTCGCCATCAAGAAGAAGGACGGAACCAAGTGGAGAAAGCTGGTGGATTTCAGGGAGCTG |
| | | AACAAGAAGACCCAGGACTTTTGGGAGGTGCAGCTGGGCATCCCCCATCCTTCAGGACTGAAGAAGAAGAAG |
| | | AGCGTGACAGTGCTGGATATCGGAGACGCTTACTTTAGCGTGCCACTGGACAAGGAGTTCAGAAAGTACACC |
| | | GCTTTCACCGTGCCTTCTACCAATAACGAGACCCCAGGAGTGAGATACCAGTATAACGTGCTGCCCATGGGT |
| | | TGGAAAGGATCTCCAGCAATTTTTCAGTGTAGCATGACA |
| 148 | Pol:542-606 | CCCAAGTTCAGGCTGCCTATCCAGAAAGAGACTTGGGACACTTGGTGGACCGATTATTGGCAGGCAACTTGG |
| | Nef:64-99 | ATTCCCGAGTGGGAATTTACCAACACACCTCCTCTGGTGAAGCTGTGGTATCAGCTGGAAACAGAGCCTATT |
| | Pol:56-117 | GCCGGAGTGGAAACATTCTACGTGGACGGAGCCAGCAACAGAGAGACCAAGGAGGAAGTGGGATTTCCAGTG |
| | Pol:932-1003 | AGACCTCAGGTGCCTCTGAGACCTATGACATACAAGGGAGCTCTGGATCTGAGCCACTTTCTGAAGGAGAAA |
| | Pol:367-431 | GGAGGACTGGAAGGACTGCCTCAGATTACTCTCTGGCAGAGACCTATCGTGACAATCAAGATCGGCGGACAG |
| | K | ATCAAGGAAGCTCTGCTGGATACAGGAGCCGACGATACAGTGCTGGAAGATATGAACCTGCCAGGCAAGTGG |
| | Pol:129-320 | AAACCTAAGATGATCGGAGGCATTGGCGGCTTTATCAAGGTGAAACAGTACGACCAGATCACCAAGCTGCAG |
| | | AACTTCAGGGTGTACTACAGGGACAACAGAGACCCCCTCTGGAAAGGACCAGCTAGACTGCTGTGGAAAGGA |
| | | GAAGGAGCAGTGGTGATCCAGGATAATAGCGAGATCAAGGTGGTGCCTAGGAGAAAGGTGAAGATCATCAGG |
| | | GACTACGGCAAGAGAATGGCAGGAGACGATTGCGTGGCAGGAAGACAGGACGAAGATTGGGGACTGACAACA |
| | | CCAGACAAGAAGCACCAGAAGGACCCCCCTTTCCTCTGGATGGGATACGAACTGCATCCAGATAGGTGGACA |
| | | GTGCAGCCAATTGAGCTGCCAGAAAAGGAGTCTTGGACAGTGAACGACATCCAGAAGCTGATCGGCAAGCTG |
| | | AATTGGGCTTCTCAGATCTACGCCGGAATTAAGGTGAAGGGAACAGTGCTGATTGGACCTACACCAGTGAAC |
| | | ATCATCGGAAGAAACCTGCTGACACAGCTGGGTTGTACACTGAACTTCCCTATCAGCCCTATCGATACCGTG |
| | | CCAGTGAAACTGAAACCAGGAATGGACGGACCTAGAGTCAAGCAGTGGCCTCTGACAGAAGAGAAGATCAAG |
| | | GCCCTGATCGAGATTTGCACCGAGATGGAGAAGGAGGGAAAGATCAGCAGAATTGGCCCCGAGAATCCTTAC |
| | | AACACCCCTATCTTCGCCATCAAGAAGAAGGACGGAACTAAGTGGAGAAAGCTGGTGGACTTCAGAGAGCTG |
| | | AACAAGAAGACCCAGGACTTTTGGGAAGTGCAGCTGGGCATCCCCCATCCTTCAGGACTGAAGAAGAAGAAG |
| | | AGCGTGACAGTGCTGGACATCGGAGACGCTTATTTTAGCGTGCCTCTGGATAAGGAGTTCCGGAAATACACC |
| | | GCCTTTACCGTGCCTTCTACCAATAACGAGACACCAGGAGTGAGGTACCAGTATAACGTGCTGCCAATGGGC |
| | | TGGAAAGGATCTCCAGCAATCTTTCAGTGTAGCATGACA |
| 149 | Pol:542-606 | CCCAAGTTCAGGCTGCCCATCCAGAAAGAAACCTGGGACACCTGGTGGACTGACTACTGGCAGGCCACCTGG |
| | Nef:64-99 | ATCCCAGAGTGGGAGTTCACCAACACCCCTCCCCTGGTGAAGCTGTGGTACCAGCTGGAAACTGAGCCCATA |
| | Pol:56-117 | GCAGGAGTGGAAACCTTTTATGTGGATGGAGCCAGCAACAGGGAAACCAAGGAAGAGGTGGGCTTCCCAGTG |
| | Pol:932-1003 | AGGCCCCAGGTGCCCCTGAGACCCATGACCTACAAGGGAGCCCTGGATCTGAGCCACTTCCTGAAAGAGAAG |
| | Pol:367-431 | GGGGGCCTGGAAGGCCTGCCCCAGATCACCCTGTGGCAGAGACCCATTGTGACCATCAAGATTGGGGGCCAG |
| | K | ATCAAAGAGGCCCTCCTGGACACTGGAGCTGATGACACAGTCCTGGAAGACATGAACCTCCCAGGAAAGTGG |
| | Pol:129-320 | AAGCCCAAGATGATTGGGGGCATTGGAGGCTTCATCAAGGTCAAGCAGTATGACCAGATCACCAAGCTCCAG |
| | | AATTTCAGAGTCTACTACAGAGACAACAGAGACCCCCTGTGGAAAGGCCCAGCCAGACTTCTCTGGAAGGGG |
| | | GAGGGAGCAGTGGTGATCCAAGACAACTCTGAGATCAAAGTGGTCCCCAGGAGAAAGGTGAAGATCATCAGG |
| | | GACTATGGCAAAAGGATGGCAGGGGATGACTGTGTGGCAGGCAGACAGGATGAGGACTGGGGCCTGACCACC |
| | | CCTGACAAGAAGCACCAGAAGGACCCCCCCTTCCTGTGGATGGGCTATGAGCTGCACCCAGACAGATGGACT |
| | | GTCCAACCCATTGAGCTGCCTGAGAAGGAGAGCTGGACAGTCAATGACATCCAGAAGCTGATTGGGAAGCTG |
| | | AATTGGGCCTCCCAGATCTATGCAGGCATCAAGGTCAAGGGCACTGTCCTGATTGGCCCCACCCCTGTGAAC |
| | | ATCATAGGCAGAAACCTGCTGACCCAGCTGGGCTGCACACTGAACTTCCCCATCAGCCCCATTGACACAGTG |
| | | CCTGTGAAGCTCAAGCCTGGCATGGATGGCCCCAGAGTGAAACAGTGGCCCTTGACAGAGGAAAAAATCAAG |
| | | GCCCTGATTGAGATCTGCACTGAGATGGAAAAAGAGGGCAAGATCTCAAGAATTGGCCCTGAGAACCCCTAC |
| | | AACACCCCCATTTTTGCCATCAAGAAAAAGGATGGCACAAAGTGGAGAAAGCTGGTGGACTTCAGAGAGCTC |
| | | AACAAGAAGACCCAGGACTTCTGGGAGGTGCAGCTGGGCATCCCCCACCCCTCTGGCCTCAAGAAGAAGAAA |
| | | AGTGTGACTGTGCTGGACATTGGTGATGCCTACTTCAGTGTCCCCCTGGACAAGGAATTCAGGAAGTACACA |
| | | GCCTTCACAGTGCCCAGCACCAACAATGAGACCCCTGGTGTCAGGTACCAGTACAATGTGCTGCCCATGGGC |
| | | TGGAAGGGCAGCCCAGCCATCTTCCAGTGCAGCATGACC |
| 150 | Pol:840-920 | ACTGTAAAGGCCGCCTGTTGGTGGGCTGGGATCAAACAGGAGTTCGGAATCCCATACAATCCCCAAAGCCAG |
| | Gag:147-369 | GGAGTAGTGGAATCCATGAATAAGGAGCTCAAGAAGATCATTGGGCAAGTCAGGGACCAGGCTGAGCACCTC |
| | Pol:586-606 | AAGACGGCCGTTCAAATGGCGGTATTTATCCATAACTTTAAACGCAAAGGCGGTATTGGCGGTTACTCCGCA |
| | AA | GGTGAGCGGATAGTTGACATTATCGCAATAAGCCCTAGGACGCTTAACGCTTGGGTCAAGGTAGTGGAGGAA |
| | Pol:683-708 | AAAGCCTTCTCACCGGAGGTCATCCCTATGTTTAGCGCCCTGAGCGAAGGAGCGACGCCACAAGACTTGAAC |
| | | ACCATGCTTAATACGGTGGGGGGTCACCAAGCTGCAATGCAAATGTTGAAGGAAACGATAAACGAAGAGGCC |
| | | GCCGAATGGGACAGGTTGCATCCCGTTCATGCAGGTCCCATAGCTCCCGGTCAAATGAGGGAACCACGCGGA |
| | | TCAGACATCGCGGGAACGACGTCCACGTTGCAGGAACAAATTGGATGGATGACCAATAACCCTCCTATCCCA |
| | | GTCGGTGAAATCTATAAGAGGTGGATCATCCTCGGCCTCAATAAAATAGTGAGGATGTATTCTCCAACTAGC |
| | | ATTCTGGATATACGCCAAGGCCCTAAAGAACCATTTCGCGATTACGTAGACCGATTTTACAAGACACTCCGG |
| | | GCTGAGCAGGCCTCCCAAGAGGTAAAGAATTGGATGACCGAAACGTTGCTGGTGCAGAATGCCAACCCCGAC |
| | | TGTAAGACCATTTTGAAGGCGCTTGGGCCAGCAGCAACACTGGAAGAGATGATGACTGCGTGTCAAGGTGTA |
| | | GGTGGCCCCGGCCACAAAGCCAGAGTGTTGGCCGAGGCAATGAGCCAAAAAGAACCCATTGTAGGAGCTGAG |
| | | ACCTTTTACGTTGATGGCGCAGCTAACAGAGAAACTAAGGCGGCAAAAGAGAAAGTTTATCTTGCATGGGTG |
| | | CCTGCGCACAAAGGAATCGGCGGAAATGAGCAAGTTGATAAACTCGTAAGT |
| 151 | Pol:840-920 | ACCGTGAAGGCGGCGTGTTGGTGGGCAGGGATAAAGCAAGAATTTGGCATACCTTATAACCCGCAGAGTCAG |
| | Pol:586-606 | GGAGTTGTCGAATCCATGAATAAGGAGCTTAAGAAGATCATCGGTCAGGTGCGCGATCAGGCAGAGCACCTC |
| | AAA | AAAACTGCGGTCCAAATGGCCGTGTTCATCCACAACTTCAAGCGCAAAGGCGGAATCGGTGGATACAGTGCT |
| | Gag:147-369 | GGGGAAAGGATCGTCGATATTATCGCCAAAGAGCCAATCGTGGGAGCGGAAACATTCTACGTAGACGGTGCG |
| | AAA | GCCAACAGGGAGACAAAAGCCGCTGCGATCTCACCCCGAACCCTTAATGCTTGGGTGAAGGTGGTGGAAGAA |
| | Pol:683-708 | AAAGCCTTCAGTCCCGAAGTTATCCCGATGTTCTCCGCCCTCAGTGAAGGTGCAACGCCGCAGGACCTTAAT |
| | | ACTATGTTGAACACCGTTGGTGGTCATCAGGCCGCCATGCAGATGCTTAAAGAAACCATTAACGAAGAAGCC |
| | | GCAGAATGGGACAGGTTGCACCCAGTCCACGCTGGGCCCATCGCGCCTGGGCAGATGCGAGAGCCACGAGGG |
| | | TCCGATATCGCGGGCACAACAAGCACTTTGCAAGAACAGATTGGGTGGATGACTAATAACCCGCCGATACCT |
| | | GTTGGGGAAATATATAAACGCTGGATAATTCTGGGACTCAATAAGATAGTGAGAATGTACTCCCCTACATCC |
| | | ATTCTTGATATACGACAAGGTCCAAAAGAACCCTTTCGCGACTACGTGGATAGATTTTATAAGACCCTCAGG |
| | | GCCGAACAGGCAAGTCAGGAGGTCAAGAACTGGATGACGGAGACGCTTCTTGTTCAGAATGCAAATCCCGAT |
| | | TGCAAAACTATCTTGAAAGCGCTCGGACCAGCAGCGACGCTGGAAGAAATGATGACCGCCTGCCAGGGCGTA |
| | | GGCGGCCCAGGTCATAAAGCAAGGGTATTGGCAGAAGCGATGAGTCAAGCAGCGGCAAAGGAGAAAGTATAT |
| | | CTTGCGTGGGTACCAGCGCACAAAGGGATAGGTGGAAACGAGCAAGTTGATAAGCTGGTCTCCCTT |
| 152 | Pol:932-1003 | ATAACGAAAATTCAGAACTTTCGAGTGTACTACAGAGATAGTAGAGACCCGCTGTGGAAGGGCCCAGCTAAA |
| | Pol:747-827 | CTCCTTTGGAAAGGTGAAGGGGCTGTGGTCATACAGGATAACTCAGACATAAAGGTCGTCCCAAGGAGAAAA |
| | Pol:129-320 | GCCAAAATAATTAGAGACTACGGCAAGCAGATGGCAGGGGATGACTGTGTTGCGAGCCGGCAGGATGAGGAT |
| | QEE | GTCGCGAAGGAAATCGTGGCGAGTTGTGATAAATGTCAACTGAAGGGTGAGGCAATGCACGGCCAAGTAGAT |
| | Nef:64-76 | TGCAGTCCAGGTATCTGGCAACTCGATTGCACCCACCTGGAGGGTAAGATTATCCTGGTGGCTGTGCATGTT |
| | | GCATCCGGCTACATCGAAGCTGAAGTGATTCCGGCTGAAACGGGGCAGGAAACCGCCTACTTCCTGTTGAAG |
| | | TTGGCTGGTCGATGGCCAGTCAAGACCGGTACAGTACTCGTTGGCCCGACGCCAGTGAATATCATAGGTCGG |
| | | AACCTGCTGACACAAATCGGGTGCACTCTTAATTTTCCGATTTCACCTATCGAAACCGTTCCAGTAAAACTC |
| | | AAGCCTGGGATGGATGGCCCGAAGGTTAAGCAATGGCCACTGACCGAAGAGAAAATCAAAGCGCTCGTGGAG |
| | | ATATGTACTGAAATGGAGAAAGAAGGAAAAATCTCTAAAATCGGGCCAGAAAATCCCTATAATACTCCGGTA |
| | | TTTGCTATCAAAAAAAAAGACTCAACCAAGTGGCGAAAGCTCGTTGACTTCCGAGAGTTGAATAAAAGGACC |
| | | CAGGATTTTTGGGAGGTTCAGCTGGGCATACCGCACCCCGCTGGCTTGAAAAAAAAGAAGTCTGTTACCGTC |
| | | CTGGATGTGGGCGACGCCTACTTCAGTGTACCTCTTGACAAAGACTTTAGAAAGTATACTGCTTTCACGATC |
| | | CCGAGTATAAACAACGAGACTCCAGGAATTAGGTACCAGTACAATGTATTGCCGCAGGGATGGAAGGGATCA |
| | | CCCGCAATCTTCCAATCTAGTATGACGCAAGAGGAGGAGGAAGTAGGTTTCCCAGTCAAACCACAAGTGCCG |
| | | CTC |
| 153 | Pol:932-1003 | ATTACTAAAATACAAAACTTCCGAGTATATTATAGGGATAGCCGGGACCCTCTCTGGAAAGGGCCCGCGAAA |
| | Pol:747-827 | CTGCTTTGGAAGGGCGAGGGCGCTGTTGTCATACAGGACAATAGCGATATAAAAGTAGTCCCGCGACGCAAA |
| | Pol:129-320 | GCAAAAATAATAAGAGATTATGGAAAACAAATGGCGGGCGACGATTGTGTCGCCTCACGGCAGGACGAGGAT |
| | QEE | GCTGCAGCCATCGGAACAGTACTCGTGGGGCCAACTCCCGTCAACATAATAGGACGAAATCTTCTGACTCAA |
| | Nef:64-76 | ATAGGTTGCACGCTCAACTTCCCAATTAGCCCTATAGAGACAGTGCCCGTAAAGTTGAAGCCTGGCATGGAC |
| | | GGCCCAAAAGTAAAGCAGTGGCCCCTTACTGAAGAGAAGATCAAGGCCCTGGTGGAGATCTGTACAGAGATG |
| | | GAGAAAGAAGGGAAAATCTCCAAAATCGGTCCAGAAAACCCATACAACACACCTGTTTTTGCGATAAAAAAA |
| | | AAGGATTCCACCAAGTGGAGGAAACTGGTGGATTTTCGAGAGTTGAATAAGCGAACCCAGGACTTCTGGGAA |
| | | GTACAGCTTGGGATACCACATCCAGCGGGTCTCAAGAAAAAGAAATCAGTTACGGTCCTCGATGTTGGCGAC |
| | | GCGTATTTTAGTGTTCCTCTCGATAAAGATTTCCGAAAATATACGGCATTCACCATACCCAGTATTAACAAC |
| | | GAGACACCGGGAATCAGGTATCAGTATAATGTACTTCCACAAGGTTGGAAGGGAAGCCCCGCGATTTTTCAG |
| | | AGCTCAATGACGGTTGCCAAGGAGATTGTAGCTAGCTGTGACAAATGTCAGCTGAAGGGCGAAGCGATGCAC |
| | | GGCCAGGTAGATTGCAGCCCCGGTATTTGGCAGCTCGACTGTACACATTTGGAAGGAAAAATTATACTTGTG |
| | | GCTGTCCACGTTGCAAGTGGATACATAGAGGCGGAGGTCATTCCCGCAGAAACTGGGCAAGAAACTGCTTAC |
| | | TTTCTCTTGAAGTTGGCCGGTAGGTGGCCAGTGAAAACCCAAGAGGAAGAGGAGGTTGGTTTTCCCGTCAAA |
| | | CCTCAAGTCCCCCTC |
| 154 | Gag:147-369 | CTGAGCCCCAGGACCCTGAATGCATGGGTCAAAGTGATTGAAGAGAAGGCTTTCTCTCCTGAAGTCATCCCA |
| | Pol:683-708 | ATGTTCACAGCACTTTCTGAGGGGGCCACACCACATGATTTGAACACCATGCTCAACACCATTGGTGGTCAT |
| | Pol:586-606 | CAGGCTGCTATGCAGATGCTCAAGGACACCATCAATGAGGAAGCAGCAGAGTGGGACAGAGTCCACCCAGTC |
| | Pol:840-920 | CATGCAGGGCCTGTTGCACCAGGGCAAATGAGGGACCCCAGAGGCTCAGACATTGCAGGTTCCACATCAACC |
| | | CTTCAAGAACAAATTGCCTGGATGACAAACAATCCCCCAATCCCAGTGGGGGACATCTACAAGAGATGGATC |
| | | ATCATGGGCCTGAACAAAATTGTGAGGATGTACAGCCCTGTGTCAATTCTGGACATCAAGCAAGGCCCAAAA |
| | | GAACCTTTCAGGGACTATGTTGACAGGTTTTACAGGACCCTCAGGGCAGAACAGGCCTCCCAGGATGTGAAG |
| | | AATTGGATGACAGAGACATTGCTTGTTCAGAACAGCAACCCAGACTGCAAGACCATTCTGAAAGCCCTGGGT |
| | | CCAGGAGCAACCCTGGAAGAGATGATGTCAGCCTGTCAGGGGGTGGGGGGGCCATCACACAAAGCCAGGGTG |
| | | TTGGCTGAGGCCATGTGCCAGAAAGAAAAAATCTATCTGGCATGGGTGCCAGCCCACAAGGGGATTGGAGGC |
| | | AATGAGCAAATTGACAAGCTGGTGTCAACTGAACCCATTGCTGGGGTAGAGACTTTTTATGTGGATGGTGCC |
| | | AGCAACAGGGAAACCAAGGCTGTGAAGGCTGCTTGTTGGTGGGCTGGAGTCAAGCAGGAGTTTGGCATTCCC |
| | | TACAACACCCAAAGCCAAGGTGTTGTTGAATCCATGAACAATGAGTTGAAAAAGATCATAGGCCAGATCAGG |
| | | GATCAAGCTGAGCATCTGAAAACTGCAGTTCAGATGGCAGTCCTGATTCACAATTTCAAGAGAAAGGGAGGG |
| | | ATAGGAGAGTACAGTGCAGGGGAAAGGATCATTGACATCATTGCA |
| 155 | Gag:147-369 | CTGAGCCCCCGAACCCTCAACGCATGGGTCAAGGTGATCGAAGAGAAGGCGTTCTCTCCTGAAGTTATTCCT |
| | Pol:683-708 | ATGTTTACGGCTCTGTCTGAGGGCGCCACACCCCATGATTTGAACACGATGCTCAACACGATAGGCGGTCAT |
| | Pol:586-606 | CAAGCCGCGATGCAGATGCTCAAGGATACGATCAACGAGGAAGCTGCAGAGTGGGACCGAGTTCACCCAGTC |
| | Pol:840-920 | CATGCAGGGCCTGTTGCACCAGGGCAAATGCGGGACCCACGCGGTTCAGATATTGCCGGTTCCACGTCTACC |
| | | CTGCAAGAACAAATAGCGTGGATGACAAATAATCCACCGATCCCGGTGGGCGATATATACAAGAGATGGATT |
| | | ATCATGGGCCTGAACAAAATAGTTAGGATGTACAGCCCGGTGTCAATACTTGATATAAAACAAGGACCGAAA |
| | | GAACCCTTTCGCGACTATGTAGACCGGTTCTACAGGACGCTTAGGGCAGAACAGGCCTCCCAAGATGTGAAG |
| | | AATTGGATGACAGAGACATTGCTTGTTCAGAACAGCAACCCTGACTGCAAGACCATACTTAAAGCTCTGGGT |
| | | CCAGGAGCCACTCTGGAAGAGATGATGTCAGCATGTCAGGGGGTAGGCGGGCCATCACATAAAGCCCGGGTT |
| | | TTGGCGGAAGCAATGTGCCAGAAAGAAAAAATTTATCTGGCGTGGGTGCCCGCCCACAAGGGGATAGGTGGT |
| | | AACGAGCAGATCGACAAGCTGGTCTCAACAGAACCTATAGCTGGAGTAGAGACGTTTTATGTGGATGGTGCA |
| | | AGCAATCGGGAAACCAAGGCGGTAAAGGCCGCTTGCTGGTGGGCCGGAGTCAAGCAGGAGTTCGGCATCCCG |
| | | TATAACACCCAAAGTCAAGGTGTCGTCGAATCCATGAATAACGAGTTGAAAAAGATCATAGGTCAAATAAGG |
| | | GATCAAGCTGAACATCTGAAGACGGCTGTTCAGATGGCGGTCCTTATCCACAACTTTAAGCGCAAAGGAGGT |
| | | ATAGGAGAGTACAGCGCAGGTGAACGGATAATAGACATTATAGCA |
| 156 | Gag:147-369 | CTGTCTCCTAGAACATTGAACGCTTGGGTTAAGGTTATAGAAGAGAAAGCCTTCAGTCCTGAGGTGATTCCC |
| | Pol:840-920 | ATGTTTACCGCCCTGAGCGAGGGCGCTACACCACATGATCTGAATACCATGCTGAATACTATCGGGGGACAT |
| | Pol:586-606 | CAGGCTGCCATGCAGATGCTTAAAGATACAATAAATGAGGAGGCTGCAGAGTGGGACAGGGTCCATCCTGTA |
| | AA | CACGCGGGACCTGTTGCGCCGGGACAGATGAGAGATCCGCGGGGGAGCGATATTGCAGGAAGCACCTCAACT |
| | Pol:683-708 | CTTCAGGAGCAGATTGCCTGGATGACGAACAACCCTCCGATTCCTGTGGGAGACATTTATAAGAGGTGGATA |
| | | ATTATGGGGTTGAACAAGATAGTCAGGATGTATTCTCCTGTTAGCATCCTGGACATAAAACAGGGCCCTAAA |
| | | GAGCCTTTTCGCGATTATGTTGACAGGTTTTATAGGACACTTCGCGCGGAGCAAGCCTCCCAGGATGTTAAA |
| | | AACTGGATGACCGAGACGCTCCTGGTTCAAAACAGTAACCCCGATTGTAAGACCATCCTTAAAGCACTTGGA |
| | | CCTGGCGCTACCCTGGAGGAAATGATGAGCGCCTGTCAGGGGGTAGGAGGCCCATCACATAAGGCACGGGTG |
| | | CTCGCAGAGGCGATGTGTCAAGCGGTGAAGGCAGCCTGCTGGTGGGCAGGTGTGAAGCAGGAATTTGGGATT |
| | | CCTTATAATACACAATCCCAAGGTGTTGTCGAATCTATGAACAATGAACTGAAGAAAATTATAGGGCAAATC |
| | | CGGGACCAAGCTGAGCACCTCAAAACCGCGGTTCAAATGGCTGTACTTATTCATAATTTCAAGCGCAAAGGG |
| | | GGAATCGGTGAGTACAGTGCTGGGGAACGGATAATAGATATTATCGCCACCGAGCCTATCGCAGGTGTTGAA |
| | | ACTTTTTACGTGGACGGTGCATCAAACAGAGAGACCAAAGCGGCCAAGGAGAAGATATATTTGGCCTGGGTT |
| | | CCTGCTCACAAGGGAATTGGCGGAAATGAGCAAATTGATAAGCTCGTAAGT |
| 157 | Pol:747-827 | GTTGCCAAGGAGATTGTGGCCTGCTGTGACAAGTGCCAATTGAAGGGGGAGGCAATTCATGGACAGGTGGAT |
| | Pol:932-1003 | TGCAGTCCAGGAGTCTGGCAGCTGGATTGCACACACCTGGAAGGGAAGGTCATCCTTGTTGCAGTTCATGTT |
| | Pol:129-320 | GCCTCAGGATACATTGAAGCAGAGATCATCCCCACAGAAACTGGTCAGGAAACAGCTTACTTCATTCTCAAA |
| | KIL | CTGGCTGGCAGGTGGCCAGTCACCACAATCACCAAGCTTCAAAATTTCAGGGTTTACTACAGGGACAACAGA |
| | QEE | GATCCTCTCTGGAAGGGACCTGCAAGACTTCTTTGGAAGGGTGAAGGGGCAGTTGTCATTCAGGACAACAGT |
| | Nef:64-76 | GAGATCAAGGTTGTTCCCAGGAGAAAGGTCAAGATCATCAGGGACTATGGGAAAAGGATGGCAGGGGATGAC |
| | | TGTGTTGCAGGGAGACAAGATGAGGATGGCACTGTGCTGATAGGCCCCACACCTGTCAACATCATTGGCAGG |
| | | AACCTCTTGACTCAGCTGGGCTGCACTCTCAATTTTCCCATTTCCCCCATTGACACAGTGCCAGTGAAACTG |
| | | AAGCCTGGAATGGATGGCCCAAGGGTCAAACAGTGGCCCCTGACAGAGGAGAAGATCAAGGCTCTCATTGAG |
| | | ATCTGCACTGAAATGGAAAAGGAAGGCAAGATCAGCAGGATTGGCCCTGAGAACCCTTACAACACTCCTATT |
| | | TTTGCAATCAAGAAGAAGGATGGGACCAAGTGGAGGAAGTTGGTTGACTTCAGGGAACTCAACAAGAAAACA |
| | | CAAGACTTTTGGGAAGTCCAACTTGGCATTCCCCATCCCAGTGGGTTGAAAAAGAAAAAGTCAGTGACAGTG |
| | | TTGGACATTGGGGATGCTTATTTCTCAGTTCCCCTTGACAAGGAGTTCAGAAAATACACAGCATTCACTGTG |
| | | CCTTCCACAAACAATGAAACCCCTGGGGTCAGGTACCAGTACAATGTCCTTCCAATGGGTTGGAAAGGCAGT |
| | | CCTGCAATCTTTCAATGCAGCATGACAAAAATCCTTCAAGAAGAAGAGGAAGTTGGCTTTCCTGTGAGGCCT |
| | | CAGGTCCCCCTT |
| 158 | Pol:747-827 | GTTGCCAAGGAGATCGTGGCGTGCTGCGATAAGTGCCAATTGAAAGGGGAGGCGATTCATGGACAGGTCGAT |
| | Pol:932-1003 | TGTAGTCCCGGAGTCTGGCAGCTGGATTGCACTCACCTGGAAGGTAAGGTCATCCTTGTGGCTGTACATGTT |
| | Pol:129-320 | GCCTCCGGGTATATTGAAGCCGAGATAATCCCTACCGAAACTGGTCAGGAAACCGCTTACTTCATTCTCAAA |
| | KIL | CTTGCTGGAAGGTGGCCCGTTACTACAATTACCAAGCTGCAAAATTTCAGGGTGTACTACAGGGATAACCGC |
| | QEE | GACCCTCTCTGGAAGGGCCCAGCACGACTGCTTTGGAAGGGTGAGGGGGCTGTTGTTATTCAGGATAACAGT |
| | Nef:64-76 | GAGATTAAGGTAGTCCCCAGGCGAAAGGTTAAAATAATACGGGACTACGGTAAAAGAATGGCGGGGGATGAC |
| | | TGTGTTGCAGGGAGACAAGACGAAGACGGAACGGTACTTATAGGCCCAACTCCGGTAAACATAATTGGTAGA |
| | | AACCTCTTGACGCAGCTGGGCTGTACTCTTAATTTTCCAATATCACCTATTGACACTGTCCCAGTCAAGCTG |
| | | AAGCCTGGAATGGATGGACCTCGGGTTAAACAGTGGCCCCTCACCGAGGAGAAGATCAAGGCGCTGATCGAA |
| | | ATCTGCACCGAAATGGAAAAGGAAGGTAAGATTAGCCGGATCGGCCCCGAGAACCCTTATAACACGCCTATA |
| | | TTCGCTATCAAGAAGAAGGACGGAACAAAGTGGAGGAAGTTGGTTGACTTCCGGGAACTTAACAAGAAAACA |
| | | CAAGACTTTTGGGAAGTCCAACTCGGCATCCCACACCCAAGTGGGTTGAAAAAGAAAAAGTCCGTAACTGTA |
| | | TTGGACATAGGTGACGCTTATTTTTCAGTACCACTTGATAAGGAATTTCGAAAATACACAGCGTTCACTGTG |
| | | CCGTCCACGAACAACGAAACCCCCGGGGTACGCTACCAATATAACGTACTGCCAATGGGTTGGAAAGGTAGT |
| | | CCTGCGATCTTTCAATGCAGTATGACTAAAATCCTTCAAGAAGAAGAGGAAGTCGGATTTCCTGTGCGGCCC |
| | | CAGGTCCCCCTG |
| 159 | Pol:129-320 | GGGACGGTGCTTATTGGGCCCACTCCAGTCAATATTATCGGACGAAACCTGCTGACTCAGCTGGGTTGCACT |
| | Pol:932-1003 | CTCAATTTCCCTATTAGTCCTATAGACACGGTGCCCGTAAAACTCAAGCCAGGCATGGATGGTCCGCGCGTG |
| | Pol:747-827 | AAGCAATGGCCTTTGACTGAAGAAAAAATTAAGGCACTCATTGAGATATGTACGGAGATGGAGAAAGAAGGG |
| | AA | AAAATCTCTCGAATTGGACCAGAAAACCCGTACAATACTCCGATTTTTGCGATTAAGAAGAAGGATGGCACG |
| | QEE | AAGTGGCGCAAACTCGTCGATTTTAGGGAGCTTAACAAGAAGACCCAAGATTTTTGGGAGGTCCAGCTGGGC |
| | Nef:64-76 | ATTCCGCATCCGTCCGGCTTGAAAAAGAAAAAATCAGTTACTGTGCTTGATATAGGAGACGCCTATTTTAGT |
| | | GTGCCGTTGGACAAGGAATTTCGCAAATATACAGCATTTACCGTCCCTTCAACGAACAATGAGACTCCGGGC |
| | | GTACGCTACCAATATAATGTGTTGCCTATGGGGTGGAAGGGGTCTCCTGCGATCTTCCAATGCTCTATGACG |
| | | ATTACCAAGCTGCAAAATTTTCGCGTATACTACCGCGACAATCGAGATCCATTGTGGAAGGGGCCGGCGAGG |
| | | CTCCTTTGGAAGGGAGAAGGTGCCGTAGTTATCCAAGATAATAGCGAGATCAAAGTTGTGCCCCGAAGAAAG |
| | | GTGAAAATTATAAGGGATTACGGGAAGAGAATGGCGGGAGACGACTGTGTTGCCGGTCGCCAAGACGAGGAT |
| | | GTTGCGAAGGAAATCGTTGCGTGTTGTGATAAATGTCAGCTGAAAGGCGAAGCAATTCACGGACAGGTAGAT |
| | | TGTTCTCCTGGCGTGTGGCAGCTTGACTGTACCCATTTGGAGGGAAAAGTGATCCTGGTAGCAGTCCACGTT |
| | | GCCAGTGGCTACATAGAAGCTGAGATAATTCCTACTGAGACGGGACAGGAGACGGCTTATTTTATTCTCAAG |
| | | CTGGCAGGCAGATGGCCCGTGACCACCGCGGCGCAGGAAGAGGAGGAAGTTGGTTTTCCCGTACGCCCTCAG |
| | | GTTCCCTTG |
| 160 | Pol:840-909 | ACAGTCAAAGCCGCTTGCTGGTGGGCGGGCATAAAGCAAGAATTTGGGATACCCTATAACCCTCAATCTCAA |
| | AA | GGGGTAGTTGAATCTATGAACAAGGAACTCAAAAAGATCATAGGACAGGTCCGCGATCAGGCTGAGCATTTG |
| | Gag:147-369 | AAGACGGCTGTGCAAATGGCGGTTTTTATCCATAACTTCAAGCGGAAGGGTGGGATCGGCGGAGCAGCAATC |
| | Pol:747-827 | AGTCCACGAACTCTTAATGCTTGGGTGAAGGTTGTTGAGGAGAAAGCGTTCAGTCCTGAGGTGATCCCCATG |
| | QEE | TTCTCTGCACTTAGCGAAGGAGCAACACCCCAGGACCTTAACACGATGTTGAACACAGTGGGGGGTCATCAA |
| | Nef:64-76 | GCCGCCATGCAGATGCTCAAAGAAACTATAAATGAGGAGGCTGCCGAGTGGGACAGACTGCATCCTGTGCAC |
| | | GCCGGACCAATAGCACCGGGGCAGATGCGAGAACCGCGAGGTTCCGACATCGCTGGGACCACTTCTACTCTG |
| | | CAGGAGCAGATTGGTTGGATGACTAACAACCCCCCGATACCGGTGGGTGAGATCTATAAGCGCTGGATCATA |
| | | CTTGGCCTTAACAAAATAGTTCGCATGTACTCACCAACAAGCATTCTCGACATCCGACAGGGGCCTAAGGAG |
| | | CCTTTTCGAGACTATGTGGATAGATTTTATAAAACTTTGCGGGCGGAGCAAGCATCCCAGGAGGTTAAGAAC |
| | | TGGATGACAGAGACACTTCTGGTCCAGAATGCCAACCCCGACTGTAAAACGATACTTAAAGCACTTGGGCCG |
| | | GCTGCAACTCTGGAGGAAATGATGACAGCGTGTCAAGGTGTGGGGGGTCCTGGCCATAAGGCTCGCGTGTTG |
| | | GCGGAAGCAATGTCACAAGTTGCCAAAGAAATAGTTGCCAGTTGCGACAAGTGCCAACTCAAAGGTGAAGCG |
| | | ATGCATGGACAGGTGGATTGCTCACCAGGCATCTGGCAGCTTGACTGTACACACCTGGAGGGCAAGATAATT |
| | | TTGGTCGCGGTGCATGTAGCAAGTGGTTATATCGAAGCTGAGGTAATACCCGCCGAGACGGGGCAAGAGACA |
| | | GCCTACTTCCTCTTGAAGTTGGCCGGTCGATGGCCGGTTAAGACGCAAGAGGAGGAAGAGGTTGGCTTCCCC |
| | | GTTAAGCCTCAAGTACCGCTT |
| 161 | Pol:840-909 | GCGGTGAAGGCCGCATGTTGGTGGGCGGGTGTTAAACAAGAGTTCGGTATACCGTACAATACGCAGAGTCAA |
| | Gag:147-369 | GGAGTCGTAGAATCTATGAATAATGAGCTGAAAAAAATCATTGGGCAAATTCGGGACCAGGCTGAGCATCTC |
| | VT | AAAACGGCCGTTCAGATGGCCGTCCTGATTCATAACTTTAAGAGAAAAGGCGGCATAGGGGAGCTCAGTCCA |
| | Pol:747-827 | AGGACTCTCAACGCCTGGGTGAAGGTTATTGAAGAAAAAGCGTTTAGCCCGGAGGTAATTCCAATGTTTACA |
| | AA | GCTCTCAGCGAGGGGGCGACACCTCATGATCTCAATACAATGCTCAATACAATAGGGGGGCACCAGGCCGCT |
| | QEE | ATGCAAATGCTGAAAGACACGATCAATGAAGAAGCGGCTGAATGGGATAGAGTTCATCCTGTTCATGCAGGA |
| | Nef:64-76 | CCGGTCGCCCCGGGACAGATGAGAGACCCGCGCGGTTCCGACATAGCTGGGAGCACGTCTACGTTGCAGGAG |
| | | CAGATCGCTTGGATGACTAATAATCCCCCTATCCCTGTCGGTGATATTTATAAACGGTGGATTATTATGGGT |
| | | TTGAACAAAATTGTGAGAATGTACAGCCCAGTTTCCATACTTGATATTAAGCAGGGGCCGAAAGAACCCTTT |
| | | AGGGACTATGTAGACCGCTTCTATCGCACACTTAGAGCCGAGCAGGCGAGTCAGGACGTAAAGAACTGGATG |
| | | ACAGAAACCCTCCTTGTCCAAAACTCCAATCCCGATTGCAAAACCATTTTGAAAGCACTCGGTCCTGGAGCC |
| | | ACTTTGGAAGAAATGATGTCCGCGTGTCAGGGGGTGGGAGGGCCAAGCCACAAAGCGAGAGTATTGGCGGAG |
| | | GCGATGTGCCAGGTTACCGTAGCGAAGGAGATAGTCGCATGCTGTGACAAATGCCAACTTAAAGGCGAGGCG |
| | | ATCCATGGTCAGGTTGACTGCAGTCCGGGGGTATGGCAACTTGACTGTACACATTTGGAGGGTAAGGTTATT |
| | | CTCGTTGCAGTTCATGTAGCTTCAGGATACATCGAGGCCGAAATCATCCCGACGGAGACGGGCCAAGAGACT |
| | | GCCTACTTCATCTTGAAACTGGCGGGTCGCTGGCCGGTAACTACCGCCGCCCAGGAGGAAGAAGAAGTTGGG |
| | | TTCCCTGTCCGACCCCAAGTGCCACTC |
| 162 | Pol:840-909 | ACGGTGAAAGCGGCCTGTTGGTGGGCGGGAATTAAGCAGGAATTTGGGATACCGTATAACCCTCAAAGCCAA |
| | Pol:747-827 | GGCGTCGTAGAATCCATGAACAAAGAGCTGAAGAAGATTATTGGCCAGGTTCGGGACCAGGCAGAACACCTT |
| | AA | AAAACAGCCGTGCAGATGGCAGTGTTCATCCATAATTTTAAGCGGAAGGGCGGGATTGGCGGAGTTGCGAAG |
| | Gag:147-369 | GAAATTGTTGCGAGTTGTGATAAATGCCAACTTAAAGGGGAGGCAATGCACGGACAAGTTGATTGCTCACCT |
| | QEE | GGCATATGGCAGCTGGATTGTACCCACCTTGAGGGTAAAATAATCCTGGTGGCCGTTCATGTCGCATCTGGC |
| | Nef:64-76 | TATATAGAAGCGGAAGTCATTCCAGCAGAGACGGGTCAAGAAACTGCTTACTTTCTCCTTAAACTTGCGGGA |
| | | AGGTGGCCTGTTAAAACCGCCGCTATTAGCCCCAGGACGTTGAATGCCTGGGTAAAGGTTGTGGAGGAGAAG |
| | | GCATTCTCCCCTGAGGTAATTCCCATGTTCTCAGCACTGAGTGAAGGGGCTACACCTCAAGATCTGAACACG |
| | | ATGCTCAACACGGTTGGCGGACATCAAGCGGCCATGCAAATGCTCAAAGAAACCATCAATGAAGAAGCGGCT |
| | | GAGTGGGACCGCCTTCATCCAGTCCATGCTGGCCCAATCGCACCTGGTCAAATGAGAGAGCCGAGGGGTAGT |
| | | GATATAGCCGGGACGACTAGCACATTGCAGGAACAGATAGGGTGGATGACAAATAACCCTCCTATACCTGTG |
| | | GGGGAAATATATAAACGCTGGATTATTCTCGGTCTGAACAAGATTGTCAGGATGTACTCCCCGACCAGTATC |
| | | CTTGACATAAGACAAGGGCCTAAGGAGCCCTTTCGGGACTACGTTGATCGGTTCTATAAGACGCTTCGGGCC |
| | | GAGCAGGCGTCTCAAGAGGTGAAAAATTGGATGACTGAAACTTTGCTGGTGCAAAATGCTAACCCCGACTGC |
| | | AAGACAATATTGAAGGCTCTCGGTCCAGCAGCAACTTTGGAAGAGATGATGACAGCGTGTCAAGGCGTAGGT |
| | | GGGCCAGGACACAAGGCTAGGGTCCTTGCAGAGGCTATGTCTCAGCAGGAGGAGGAAGAGGTAGGTTTCCCC |
| | | GTCAAGCCTCAAGTCCCACTC |
| 163 | Gag:147-369 | CTTAGTCCGAGAACTCTCAACGCTTGGGTCAAAGTCATAGAGGAGAAAGCCTTTTCACCCGAAGTAATACCT |
| | PV | ATGTTCACTGCGTTGAGCGAGGGCGCGACACCTCATGACCTGAATACTATGCTGAACACCATCGGGGGCCAC |
| | Pol:747-827 | CAAGCAGCTATGCAGATGCTGAAGGACACAATTAACGAAGAAGCGGCAGAATGGGATAGGGTTCACCCTGTA |
| | AAY | CATGCCGGACCAGTTGCACCTGGCCAAATGAGAGACCCACGAGGGAGCGACATCGCAGGCTCAACTAGTACC |
| | Pol:840-909 | CTGCAAGAGCAGATAGCGTGGATGACCAATAATCCTCCTATTCCTGTTGGTGACATTTACAAACGATGGATA |
| | QEE | ATAATGGGCCTCAATAAGATCGTCAGAATGTACAGCCCAGTGAGCATCCTGGATATAAAGCAGGGACCGAAA |
| | Nef:64-76 | GAACCCTTCCGGGACTATGTTGACCGCTTCTACCGGACTCTTAGGGCGGAACAGGCCAGCCAGGACGTAAAA |
| | | AACTGGATGACTGAAACGTTGTTGGTTCAAAATTCAAATCCAGACTGCAAAACCATTCTCAAAGCACTCGGA |
| | | CCAGGCGCTACCCTGGAAGAGATGATGTCTGCCTGTCAAGGTGTCGGGGGGCCGAGTCACAAGGCACGCGTA |
| | | CTGGCGGAGGCCATGTGTCAACCAGTAGTTGCCAAAGAGATTGTCGCGTGCTGTGATAAGTGTCAGCTCAAA |
| | | GGGGAAGCGATACATGGACAAGTAGACTGTAGTCCTGGCGTGTGGCAGTTGGACTGTACCCATTTGGAGGGC |
| | | AAGGTAATATTGGTAGCTGTCCATGTCGCGTCTGGTTATATCGAAGCAGAAATCATTCCGACTGAGACTGGT |
| | | CAAGAGACGGCCTACTTCATACTGAAACTTGCAGGTAGGTGGCCGGTAACAACGGCGGCTTATGCCGTAAAA |
| | | GCTGCTTGTTGGTGGGCTGGAGTCAAGCAAGAATTTGGAATCCCTTACAACACACAGAGTCAAGGCGTCGTC |
| | | GAGTCTATGAATAACGAGCTGAAAAAGATCATAGGCCAAATCAGAGACCAAGCCGAACACTTGAAGACAGCC |
| | | GTTCAAATGGCAGTTCTTATCCACAACTTCAAGCGCAAAGGGGGCATAGGTGAACAGGAGGAGGAGGAGGTA |
| | | GGCTTCCCTGTCCGGCCGCAAGTGCCGCTC |
| 164 | Pol:840-920 | ACAGTGAAGGCAGCTTGTTGGTGGGCCGGAATTAAACAGGAGTTTGGCATCCCTTATAATCCTCAGTCTCAG |
| | Gag:147-369 | GGAGTGGTGGAGTCTATGAACAAGGAGCTGAAGAAGATCATCGGCCAGGTGAGAGATCAGGCAGAACATCTG |
| | Gag:1-53 | AAGACAGCAGTGCAGATGGCCGTGTTTATCCACAACTTCAAGAGGAAGGGCGGCATTGGAGGATATAGCGCA |
| | PPV | GGAGAAAGAATCGTGGACATCATCGCCATCTCTCCTAGAACACTGAACGCTTGGGTGAAAGTGGTGGAGGAG |
| | Pol:747-827 | AAAGCCTTTAGCCCAGAAGTGATCCCTATGTTCTCAGCTCTGTCAGAAGGAGCTACACCTCAGGATCTGAAC |
| | Pol:683-708 | ACCATGCTGAATACCGTGGGAGGACATCAGGCAGCTATGCAGATGCTGAAGGAGACAATTAACGAGGAAGCA |
| | Nef:117-148 | GCCGAGTGGGATAGACTGCATCCAGTGCACGCAGGACCTATTGCTCCAGGACAGATGAGAGAGCCTAGAGGA |
| | RAKR | AGCGATATTGCCGGCACAACATCTACACTGCAGGAACAGATCGGTTGGATGACCAACAATCCTCCTATCCCA |
| | F2A linker | GTGGGCGAAATCTACAAACGCTGGATCATCCTGGGCCTGAATAAGATCGTGAGAATGTACAGCCCCACAAGC |
| | Gag:147-369 | ATCCTGGATATCAGACAGGGACCTAAGGAACCTTTCAGGGATTACGTGGACCGGTTCTACAAGACACTGAGA |
| | Pol:747-827 | GCAGAACAGGCATCTCAGGAGGTGAAGAATTGGATGACCGAGACACTGCTGGTGCAGAACGCTAATCCAGAT |
| | Gag:1-53 | TGCAAGACCATTCTGAAAGCTCTGGGACCAGCAGCTACACTGGAAGAGATGATGACAGCTTGTCAGGGAGTG |
| | AA | GGAGGACCAGGACATAAAGCTAGAGTGCTGGCAGAAGCTATGTCTCAGATGGCAGCTAGAGCTTCAGTGCTG |
| | Pol:840-920 | TCAGGAGGAGAACTCGATAGGTGGGAGAAGATCAGACTGAGACCAGGAGGCAAGAAGAAGTACAGACTGAAG |
| | Nef:117-148 | CACATCGTGTGGGCTTCTAGAGAACTGGAGAGATTTGCCGTGAATCCAGGACTCCTGGAAACACCTCCAGTG |
| | LIK | GTGGCTAAAGAGATTGTGGCTTCTTGCGATAAGTGCCAGCTGAAAGGAGAGGCTATGCACGGACAGGTGGAT |
| | Pol:683-708 | TGTTCTCCAGGAATTTGGCAGCTGGATTGTACACACCTGGAGGGAAAGATTATTCTGGTGGCAGTGCACGTG |
| | | GCATCAGGATATATTGAGGCCGAAGTGATTCCAGCAGAAACAGGACAGGAGACAGCTTACTTTCTGCTCAAA |
| | | CTGGCAGGTCGCTGGCCAGTGAAAACCAAGGAGAAGGTGTACCTGGCTTGGGTGCCAGCTCATAAAGGAATT |
| | | GGCGGAAACGAGCAGGTGGATAAACTGGTGTCTACACAGGGCTACTTCCCAGATTGGCAGAATTACACACCA |
| | | GGACCAGGCACAAGATATCCTCTGACATTCGGTTGGTGTTTCAAGCTCGTGCCAGTGAGAGCTAAAAGAGCT |
| | | CCAGTGAAGCAGACCCTGAATTTCGATCTGCTGAAGCTCGCAGGAGACGTGGAATCTAATCCAGGACCTCTG |
| | | TCTCCTAGAACACTGAACGCTTGGGTGAAGGTGATCGAAGAGAAGGCCTTTAGCCCAGAAGTGATCCCTATG |
| | | TTTACAGCCCTGAGCGAAGGAGCTACACCTCACGATCTGAATACCATGCTGAACACAATTGGAGGACATCAG |
| | | GCCGCTATGCAGATGCTGAAGGACACCATCAACGAAGAAGCAGCCGAGTGGGATAGAGTGCATCCAGTGCAC |
| | | GCAGGACCAGTGGCTCCAGGACAGATGAGAGATCCTAGAGGAAGCGACATCGCAGGATCTACATCTACACTG |
| | | CAGGAACAGATCGCCTGGATGACAAATAACCCCCCTATCCCAGTGGGAGATATCTATAAGCGCTGGATCATC |
| | | ATGGGCCTGAACAAGATCGTGAGGATGTACAGCCCAGTGTCTATCCTGGACATCAAGCAGGGACCTAAGGAG |
| | | CCTTTTAGAGACTACGTGGACAGATTCTACAGAACACTGAGAGCCGAACAGGCATCTCAGGACGTGAAGAAT |
| | | TGGATGACCGAGACACTGCTGGTGCAGAACAGCAACCCCGATTGCAAGACAATCCTGAAAGCCCTGGGACCA |
| | | GGAGCTACACTGGAAGAGATGATGTCAGCTTGTCAGGGAGTGGGAGGACCATCTCATAAGGCTAGAGTGCTG |
| | | GCAGAAGCTATGTGTCAGGTGGCTAAGGAGATTGTGGCTTGTTGCGACAAGTGCCAGCTGAAAGGAGAAGCT |
| | | ATCCACGGACAGGTGGATTGTTCTCCAGGAGTCTGGCAGCTGGATTGTACACACCTGGAAGGAAAAGTGATT |
| | | CTGGTGGCAGTGCACGTGGCCAGCGGCTATATTGAAGCCGAGATCATTCCTACAGAGACAGGACAGGAGACA |
| | | GCTTACTTCATTCTGAAACTGGCAGGTCGCTGGCCAGTGACAACAATGGCAGCTAGAGCTTCTATCCTGTCA |
| | | GGAGGAAAGCTCGATAAGTGGGAAAAGATCAGACTGAGACCAGGCGGAAGAAAGAAGTACAAGCTGAAGCAC |
| | | CTCGTCTGGGCTTCTAGAGAACTGGAAAGATTCGCCCTGAATCCAGGTCTGCTGGAAACAGCAGCAGCAGTG |
| | | AAAGCAGCTTGTTGGTGGGCAGGAGTGAAACAGGAGTTCGGCATCCCTTACAACACCCAGTCTCAGGGAGTG |
| | | GTGGAATCTATGAACAACGAGCTGAAGAAGATCATCGGCCAGATCAGAGACCAGGCAGAACATCTGAAGACA |
| | | GCAGTGCAGATGGCAGTGCTGATTCACAACTTCAAGAGAAAGGGCGGCATTGGAGAGTATAGCGCCGGAGAG |
| | | AGAATCATCGATATCATTGCCACACAGGGCTTCTTCCCAGATTGGCAGAATTACACCCCAGGACCAGGCATT |
| | | AGATTTCCTCTGACCTTCGGTTGGTGTTTCAAACTGGTGCCCCTGCTGATCAAGAAGGAGAAGATCTACCTG |
| | | GCTTGGGTGCCAGCTCATAAAGGAATCGGAGGAAACGAGCAGATCGATAAACTGGTGTCT |
| 165 | Pol:56-117 | TTTCCTCAGATCACTCTCTGGCAGAGACCACTGGTGACAATCAAGATCGGAGGACAGCTGAAAGAAGCTCTG |
| | Nef:64-99 | CTGGATACAGGAGCAGACGATACAGTGCTGGAAGAGATGAATCTGCCAGGTCGCTGGAAACCTAAGATGATT |
| | LI | GGAGGCATTGGCGGCTTTATCAAGGTGAGACAGTACGACCAGGAGGAAGTGGGATTTCCAGTGAAACCTCAG |
| | Pol:542-606 | GTGCCTCTGAGACCTATGACATTTAAGGGCGCTCTGGACCTGTCTCACTTTCTGAGAGAGAAGGGAGGACTG |
| | Pol:367-431 | GAAGGACTGATCCCTAAGTTCAAGCTGCCTATCCAGAAGGAGACTTGGGAAACTTGGTGGACAGAGTATTGG |
| | Pol:932-1003 | CAGGCTACTTGGATTCCCGAGTGGGAATTTGTGAACACACCTCCTCTGGTGAAGCTGTGGTATCAGCTGGAA |
| | Pol:129-320 | AAGGAGCCTATTGTGGGCGCAGAAACATTCTACGTGGACGGAGCAGCTAACAGAGAAACTAAGTGGGGATTC |
| | RAKR | ACCACCCCAGATAAGAAGCACCAGAAGGAGCCACCATTTCTCTGGATGGGATACGAACTGCACCCAGATAAG |
| | F2A linker | TGGACAGTCCAGCCTATTGTGCTGCCAGAAAAGGACTCTTGGACCGTGAACGATATCCAGAAGCTGGTGGGA |
| | Pol:56-117 | AAGCTGAATTGGGCTTCTCAGATCTACCCAGGAATCAAGGTGATCACCAAGATCCAGAACTTCAGGGTGTAC |
| | Pol:367-431 | TACAGAGACAGCAGAGATCCTCTCTGGAAGGGACCAGCTAAACTCCTCTGGAAAGGAGAAGGAGCAGTGGTG |
| | AA | ATCCAGGATAACAGCGACATCAAGGTGGTGCCTAGAAGAAAGGCCAAGATCATCAGGGACTACGGCAAACAG |
| | QEE | ATGGCAGGAGACGATTGCGTGGCTTCTAGACAGGACGAAGACGGAACAGTCCTGGTGGGACCTACACCAGTG |
| | Nef:64-99 | AATATCATCGGCAGAAATCTCCTGACACAGATCGGTTGTACCCTGAACTTCCCTATCAGCCCTATCGAGACA |
| | Pol:932-1003 | GTGCCAGTGAAACTGAAGCCAGGAATGGACGGACCTAAAGTCAAGCAGTGGCCTCTGACAGAAGAGAAGATC |
| | Pol:542-606 | AAGGCCCTGGTGGAGATTTGCACAGAGATGGAGAAGGAGGGAAAGATCAGCAAGATCGGCCCAGAGAATCCT |
| | AA | TACAACACCCCAGTGTTCGCCATCAAGAAGAAGGATAGCACCAAGTGGAGAAAGCTGGTGGATTTCAGGGAG |
| | Pol:129-320 | CTGAACAAGAGAACCCAGGACTTTTGGGAAGTGCAGCTGGGCATCCCCCATCCAGCAGGACTGAAGAAGAAG |
| | | AAGAGCGTGACAGTGCTGGACGTGGGAGACGCTTATTTTAGCGTGCCTCTGGACAAGGACTTCAGAAAGTAC |
| | | ACCGCCTTCACCATCCCTTCTATCAACAACGAGACCCCAGGCATCAGATACCAGTATAACGTGCTGCCTCAG |
| | | GGTTGGAAAGGATCTCCAGCAATCTTCCAGAGCAGCATGACCAGAGCTAAGAGAGCTCCAGTGAAACAGACC |
| | | CTGAACTTCGATCTGCTCAAACTGGCAGGAGACGTGGAAAGCAATCCAGGACCTAATCTGCCTCAGATCACA |
| | | CTGTGGCAGAGACCTATTGTGACCATCAAGATTGGCGGCCAGATTAAAGAAGCCCTGCTGGATACAGGAGCA |
| | | GACGATACAGTGCTGGAGGATATGAACCTGCCAGGTAAGTGGAAGCCTAAGATGATTGGCGGAATTGGCGGC |
| | | TTTATCAAGGTCAAGCAGTACGATCAGTGGGGACTGACAACACCAGACAAGAAGCACCAGAAGGACCCCCCA |
| | | TTCCTCTGGATGGGATACGAACTGCATCCAGATAGGTGGACAGTGCAGCCAATTGAACTGCCAGAGAAGGAG |
| | | TCTTGGACCGTGAACGATATCCAGAAGCTGATCGGCAAGCTGAATTGGGCTTCTCAGATCTACGCAGGAATT |
| | | AAGGTGGCCGCTCAGGAAGAAGAGGAAGTGGGATTTCCAGTGAGACCTCAGGTGCCTCTGAGACCTATGACA |
| | | TACAAGGGAGCTCTGGACCTGTCTCACTTTCTGAAGGAGAAGGGAGGACTGGAAGGAATCACCAAGCTGCAG |
| | | AATTTCCGGGTGTACTACAGGGACAATAGAGACCCTCTCTGGAAAGGACCAGCTAGACTGCTGTGGAAAGGA |
| | | GAAGGAGCAGTGGTGATCCAGGATAATAGCGAGATCAAGGTGGTGCCTAGGAGAAAGGTGAAGATCATCAGG |
| | | GACTACGGCAAGAGAATGGCAGGAGACGATTGCGTGGCAGGAAGACAGGACGAGGACCCCAAGTTCAGACTG |
| | | CCTATCCAGAAGGAGACTTGGGATACTTGGTGGACCGATTATTGGCAGGCAACTTGGATTCCCGAGTGGGAG |
| | | TTTACAAATACCCCTCCTCTGGTCAAGCTCTGGTATCAGCTGGAAACAGAGCCTATTGCCGGAGTGGAAACC |
| | | TTTTACGTGGACGGAGCCAGCAACAGAGAGACAAAAGCCGCAGGAACAGTGCTGATTGGACCTACCCCAGTG |
| | | AACATCATCGGAAGAAACCTGCTGACACAGCTGGGTTGTACACTGAACTTCCCTATCAGCCCTATTGATACA |
| | | GTGCCAGTGAAGCTGAAACCAGGAATGGACGGACCTAGAGTCAAGCAGTGGCCTCTGACAGAAGAGAAGATC |
| | | AAGGCCCTGATCGAGATTTGCACCGAAATGGAGAAGGAGGGCAAGATTAGCAGGATCGGCCCAGAGAATCCT |
| | | TACAATACCCCTATCTTCGCCATCAAGAAGAAGGACGGCACCAAGTGGAGAAAACTGGTGGATTTCAGGGAG |
| | | CTGAACAAGAAGACCCAGGACTTTTGGGAGGTGCAGCTGGGCATCCCCCATCCTTCAGGACTGAAGAAGAAG |
| | | AAGAGCGTGACAGTGCTGGACATTGGAGACGCTTACTTTAGCGTGCCACTGGACAAGGAGTTCAGAAAGTAC |
| | | ACCGCCTTCACAGTGCCTAGCACAAATAACGAGACCCCAGGAGTGAGATACCAGTATAACGTGCTGCCAATG |
| | | GGCTGGAAAGGAAGCCCAGCTATCTTTCAGTGTAGCATGACA |
| 166 | Pol:747-827 | GTGGCCAAAGAAATTGTGGCCTCTTGCGATAAGTGCCAGCTGAAAGGAGAGGCTATGCACGGACAGGTGGAT |
| | Nef:117-148 | TGTTCTCCAGGAATTTGGCAGCTGGATTGTACACACCTGGAGGGAAAGATTATTCTGGTGGCAGTGCACGTG |
| | Pol:840-920 | GCATCAGGATATATTGAGGCCGAAGTGATTCCAGCAGAAACAGGACAGGAGACAGCTTACTTTCTGCTCAAA |
| | AA | CTGGCAGGTCGCTGGCCAGTGAAGACAACACAGGGCTACTTTCCTGATTGGCAGAATTACACACCAGGACCA |
| | Gag:1-53 | GGAACAAGATACCCTCTGACCTTTGGTTGGTGCTTCAAACTGGTGCCCGTGACAGTGAAAGCAGCTTGTTGG |
| | SEG | TGGGCAGGAATTAAGCAGGAGTTCGGCATCCCTTACAATCCTCAGTCTCAGGGAGTGGTGGAATCTATGAAC |
| | Gag:147-369 | AAGGAGCTGAAGAAGATCATCGGCCAGGTGAGAGATCAGGCAGAACATCTGAAGACAGCAGTGCAGATGGCA |
| | AAA | GTGTTCATCCACAACTTCAAGCGGAAGGGAGGAATTGGAGGATATAGCGCAGGAGAGAGAATCGTGGATATC |
| | Pol:683-708 | ATTGCCGCCGCTATGGCAGCTAGAGCCAGCGTGCTGAGCGGAGGAGAACTCGATCGCTGGGAAAAGATCAGA |
| | RAKR | CTGAGACCAGGAGGCAAGAAGAAGTACAGACTGAAGCACATCGTCTGGGCTTCTAGAGAACTGGAGAGATTT |
| | F2A linker | GCCGTGAATCCAGGACTGCTGGAAACAAGCGAGGGCATTTCTCCTAGAACCCTGAACGCTTGGGTGAAAGTG |
| | Gag:147-369 | GTGGAAGAAAAAGCCTTCTCTCCAGAGGTGATCCCTATGTTTAGCGCTCTGTCAGAAGGAGCTACACCTCAG |
| | Pol:840-920 | GATCTGAACACCATGCTGAACACAGTGGGAGGACATCAGGCAGCTATGCAGATGCTGAAGGAGACAATTAAC |
| | Pol:683-708 | GAAGAAGCCGCCGAGTGGGATAGACTGCATCCAGTGCACGCAGGACCTATTGCTCCAGGACAGATGAGAGAG |
| | AAY | CCTAGAGGAAGCGATATTGCCGGAACAACAAGCACACTGCAGGAACAGATCGGTTGGATGACCAATAATCCC |
| | Gag:1-53 | CCTATTCCAGTGGGCGAGATCTATAAGCGCTGGATTATCCTGGGCCTGAACAAGATCGTGAGAATGTACAGC |
| | Nef:117-148 | CCCACCTCTATCCTGGATATCAGACAGGGCCCTAAGGAACCTTTCAGAGACTACGTGGACAGGTTCTACAAG |
| | Pol:747-827 | ACACTGAGAGCAGAACAGGCATCTCAGGAGGTGAAGAATTGGATGACCGAGACACTGCTGGTGCAGAACGCC |
| | | AATCCAGATTGCAAGACAATTCTGAAAGCCCTGGGACCAGCAGCTACACTGGAAGAGATGATGACCGCTTGT |
| | | CAGGGAGTGGGAGGACCAGGACATAAAGCTAGAGTGCTGGCAGAAGCTATGTCTCAGGCAGCAGCTAAGGAG |
| | | AAAGTGTATCTGGCTTGGGTGCCAGCCCATAAAGGAATTGGAGGAAACGAGCAGGTGGATAAGCTGGTGTCT |
| | | AGAGCTAAGAGAGCTCCAGTGAAGCAGACCCTGAACTTTGATCTGCTCAAGCTGGCAGGAGACGTGGAATCT |
| | | AATCCAGGACCTCATCAGGCTCTGTCTCCTAGAACACTGAACGCTTGGGTGAAGGTGATCGAAGAGAAGGCC |
| | | TTTAGCCCAGAAGTGATCCCTATGTTTACAGCCCTGAGCGAAGGAGCTACACCTCACGATCTGAATACCATG |
| | | CTGAACACAATTGGAGGACATCAGGCCGCTATGCAGATGCTGAAGGACACCATCAACGAGGAAGCAGCAGAG |
| | | TGGGATAGAGTGCATCCAGTGCATGCAGGACCAGTGGCTCCAGGACAGATGAGAGATCCTAGAGGAAGCGAT |
| | | ATCGCAGGATCTACAAGCACACTGCAGGAACAGATCGCTTGGATGACCAATAACCCACCTATCCCAGTGGGA |
| | | GACATCTACAAGCGCTGGATCATCATGGGACTGAACAAGATCGTGAGGATGTACAGCCCAGTGTCTATCCTG |
| | | GATATCAAGCAGGGACCTAAGGAGCCTTTCAGAGATTACGTGGACAGGTTTTACAGAACCCTGAGAGCCGAA |
| | | CAGGCATCTCAGGACGTGAAGAATTGGATGACCGAAACACTGCTGGTGCAGAATAGCAACCCAGATTGCAAG |
| | | ACAATCCTGAAAGCCCTGGGACCAGGAGCTACACTGGAAGAAATGATGAGCGCTTGTCAGGGAGTGGGAGGA |
| | | CCATCTCATAAGGCTAGAGTGCTGGCAGAAGCTATGTGTCAGGCAGTGAAAGCAGCTTGTTGGTGGGCAGGA |
| | | GTGAAACAGGAGTTTGGCATCCCTTACAACACACAGTCTCAGGGAGTGGTGGAATCTATGAACAACGAGCTG |
| | | AAGAAGATCATCGGCCAGATCAGAGACCAGGCAGAACATCTGAAGACAGCAGTGCAGATGGCAGTGCTGATT |
| | | CACAACTTCAAGAGAAAGGGCGGCATTGGAGAGTATAGCGCCGGCGAGAGAATTATCGACATCATCGCCAAG |
| | | GAGAAGATCTATCTGGCTTGGGTGCCAGCCCATAAAGGAATTGGAGGCAATGAGCAGATCGATAAACTGGTG |
| | | TCAGCAGCTTATATGGCCGCTAGAGCCTCTATTCTGAGCGGAGGAAAGCTCGATAAGTGGGAGAAGATCAGA |
| | | CTGAGACCAGGAGGCAGAAAGAAGTACAAGCTGAAGCATCTCGTCTGGGCTTCTAGAGAACTGGAGAGATTC |
| | | GCTCTGAATCCAGGACTGCTGGAAACAACACAGGGCTTCTTCCCCGATTGGCAGAATTACACACCAGGACCA |
| | | GGAATCAGATTCCCTCTGACCTTCGGTTGGTGTTTTAAGCTGGTGCCTCTGGTGGCTAAGGAAATTGTGGCT |
| | | TGTTGCGACAAGTGCCAGCTGAAAGGAGAAGCTATCCACGGACAGGTGGATTGTTCTCCAGGAGTCTGGCAG |
| | | CTGGATTGTACACACCTGGAGGGAAAAGTGATTCTGGTGGCAGTGCACGTGGCATCAGGATATATTGAGGCC |
| | | GAGATCATTCCTACAGAAACAGGACAGGAGACAGCCTACTTTATCCTGAAGCTGGCTGGTAGGTGGCCAGTG |
| | | ACAACA |
| 167 | Pol:932-1003 | ATCACCAAGATCCAGAACTTCAGGGTGTACTACAGAGACAGCAGAGATCCTCTCTGGAAGGGACCAGCTAAA |
| | AAY | CTCCTCTGGAAAGGAGAAGGAGCAGTGGTGATCCAGGATAACAGCGACATCAAGGTGGTGCCTAGAAGAAAG |
| | EE | GCCAAGATCATCAGGGACTACGGCAAACAGATGGCAGGAGACGATTGCGTGGCTTCTAGACAGGACGAAGAC |
| | Nef:64-99 | GCAGCTTACGAAGAGGAAGAGGTGGGATTTCCAGTGAAACCTCAGGTGCCTCTGAGACCTATGACATTCAAG |
| | AA | GGAGCTCTGGATCTGTCTCACTTCCTGAGAGAAAAGGGAGGACTGGAAGGAGCAGCTTGGGGATTTACCACC |
| | Pol:367-431 | CCAGACAAGAAGCACCAGAAGGAACCACCATTCCTCTGGATGGGATACGAACTGCACCCAGATAAGTGGACA |
| | Pol:542-606 | GTCCAGCCTATTGTGCTGCCAGAAAAGGACTCTTGGACCGTGAACGATATCCAGAAGCTGGTGGGAAAGCTG |
| | Pol:56-117 | AATTGGGCTTCTCAGATCTACCCAGGAATCAAGGTGCCCAAGTTCAAGCTGCCTATCCAGAAGGAGACTTGG |
| | Pol:129-320 | GAAACTTGGTGGACAGAGTATTGGCAGGCTACTTGGATTCCCGAGTGGGAATTTGTGAACACACCTCCTCTG |
| | RAKR | GTGAAGCTGTGGTATCAGCTGGAAAAGGAGCCTATCGTGGGCGCAGAAACATTTTACGTGGACGGAGCCGCC |
| | F2A linker | AATAGAGAAACCAAGTTTCCTCAGATCACTCTCTGGCAGAGACCTCTGGTGACAATCAAGATCGGCGGACAG |
| | Pol:542-606 | CTGAAAGAGGCTCTGCTGGATACAGGAGCAGACGATACCGTGCTGGAAGAAATGAATCTGCCAGGTAGGTGG |
| | Nef:64-99 | AAGCCTAAGATGATTGGCGGAATTGGCGGCTTCATCAAGGTGAGACAGTACGATCAGGGAACAGTGCTGGTG |
| | Pol:56-117 | GGACCTACTCCAGTGAACATCATCGGAAGGAACCTGCTGACACAGATCGGCTGTACACTGAACTTCCCTATC |
| | Pol:932-1003 | AGCCCTATCGAGACAGTGCCAGTGAAACTGAAGCCAGGAATGGACGGACCTAAAGTCAAACAGTGGCCTCTG |
| | Pol:367-431 | ACAGAGGAGAAAATCAAAGCCCTGGTGGAGATTTGTACCGAGATGGAGAAGGAGGGCAAGATTTCTAAGATC |
| | K | GGACCAGAGAACCCCTACAATACCCCAGTGTTTGCCATCAAGAAGAAGGACAGCACCAAGTGGAGGAAGCTG |
| | Pol:129-320 | GTGGATTTTAGGGAGCTGAACAAGAGGACACAGGACTTTTGGGAAGTGCAGCTGGGCATCCCCCACCCAGCC |
| | | GGACTGAAAAAGAAGAAGTCAGTGACAGTGCTGGACGTGGGAGATGCCTATTTTAGCGTGCCTCTGGATAAG |
| | | GACTTCAGGAAGTACACCGCCTTCACAATCCCTAGCATCAACAACGAGACCCCAGGAATCAGATACCAGTAC |
| | | AACGTGCTGCCTCAGGGTTGGAAAGGATCTCCAGCCATCTTTCAGAGCAGCATGACAAGAGCCAAGAGAGCT |
| | | CCAGTGAAGCAGACCCTGAATTTCGATCTGCTGAAGCTCGCAGGAGACGTGGAATCTAATCCAGGACCTCCC |
| | | AAGTTCAGACTGCCTATTCAGAAGGAGACTTGGGACACTTGGTGGACCGATTATTGGCAGGCAACTTGGATT |
| | | CCCGAGTGGGAGTTCACAAATACACCTCCTCTGGTCAAGCTCTGGTATCAGCTGGAAACAGAGCCTATCGCA |
| | | GGAGTGGAAACATTCTACGTGGACGGAGCTTCTAACAGAGAGACCAAGGAGGAGGTGGGATTTCCAGTGAGA |
| | | CCTCAGGTGCCTCTGAGACCTATGACATACAAGGGAGCCCTGGATCTGTCTCACTTTCTGAAGGAGAAAGGC |
| | | GGACTGGAAGGACTGCCTCAGATTACTCTCTGGCAGAGGCCTATTGTGACAATCAAGATCGGCGGACAGATC |
| | | AAAGAAGCCCTGCTGGATACAGGAGCAGACGATACAGTGCTGGAGGATATGAACCTGCCAGGCAAGTGGAAA |
| | | CCTAAGATGATCGGAGGAATCGGCGGATTTATCAAGGTGAAGCAGTACGACCAGATCACCAAGCTGCAGAAC |
| | | TTCAGGGTGTACTACAGAGACAACAGAGACCCTCTCTGGAAAGGACCAGCTAGACTCCTCTGGAAAGGAGAA |
| | | GGAGCAGTGGTGATCCAGGATAATAGCGAGATCAAGGTGGTGCCTAGGAGAAAGGTGAAGATCATCCGGGAC |
| | | TACGGCAAAAGAATGGCAGGAGACGATTGCGTGGCAGGAAGACAGGACGAAGATTGGGGACTGACAACCCCA |
| | | GATAAGAAGCACCAGAAGGACCCCCCATTCCTCTGGATGGGATACGAACTGCATCCAGATAGGTGGACAGTG |
| | | CAGCCAATTGAACTGCCAGAAAAGGAGTCTTGGACAGTGAACGACATCCAGAAGCTGATCGGCAAGCTGAAT |
| | | TGGGCTTCTCAGATCTACGCCGGAATTAAGGTGAAGGGAACAGTGCTGATTGGACCTACACCAGTGAACATC |
| | | ATCGGGAGAAACCTGCTGACACAGCTGGGTTGTACACTGAACTTCCCTATCAGCCCTATCGATACAGTGCCA |
| | | GTGAAACTGAAGCCAGGAATGGACGGACCTAGAGTGAAACAGTGGCCTCTGACAGAAGAGAAGATCAAGGCC |
| | | CTGATCGAGATTTGTACAGAGATGGAGAAGGAGGGCAAGATCTCTAGAATTGGCCCAGAGAACCCCTACAAT |
| | | ACCCCTATCTTTGCCATCAAGAAGAAGGACGGCACCAAGTGGAGAAAGCTGGTGGATTTCAGGGAGCTGAAC |
| | | AAGAAGACCCAGGACTTTTGGGAAGTGCAGCTGGGCATCCCCCACCCTAGCGGACTGAAAAAGAAGAAGAGC |
| | | GTGACCGTGCTGGATATTGGAGACGCCTATTTTAGCGTGCCACTGGATAAGGAGTTCAGAAAGTACACCGCC |
| | | TTTACCGTGCCTTCTACCAATAACGAGACACCAGGAGTGAGATACCAGTACAACGTGCTGCCTATGGGTTGG |
| | | AAGGGATCACCAGCCATCTTTCAGTGTAGCATGACA |
| 210 | Pol:840-920 | ACGGTGAAGGCTGCGTGTTGGTGGGCCGGCATAAAACAAGAATTTGGAATACCATACAATCCACAATCTCAA |
| | Gag:147-369 | GGCGTTGTGGAATCCATGAATAAAGAATTGAAAAAAATTATCGGGCAAGTTCGAGACCAGGCTGAGCATCTC |
| | Pol:586-606 | AAAACGGCCGTACAGATGGCGGTGTTTATTCACAACTTCAAAAGAAAAGGAGGAATCGGTGGTTACAGTGCA |
| | AA | GGCGAACGAATAGTTGACATTATAGCGATATCTCCTCGGACTCTGAATGCGTGGGTAAAGGTAGTCGAGGAG |
| | Pol:683-708 | AAAGCATTTAGCCCCGAAGTCATCCCCATGTTTTCAGCCCTTTCAGAGGGCGCTACACCACAGGATCTGAAT |
| | RAKR | ACCATGCTGAACACAGTAGGGGGGCACCAAGCGGCGATGCAGATGCTGAAGGAGACAATAAATGAGGAGGCG |
| | F2A linker | GCGGAATGGGATAGATTGCATCCCGTCCACGCGGGGCCGATAGCGCCTGGCCAGATGAGGGAGCCACGAGGT |
| | Gag:147-369 | TCCGACATCGCGGGAACAACCTCAACCCTGCAGGAACAAATAGGGTGGATGACGAATAACCCTCCTATTCCA |
| | Pol:840-920 | GTTGGTGAAATTTATAAACGATGGATAATACTCGGTCTCAATAAAATAGTAAGGATGTATTCTCCGACAAGC |
| | Pol:586-606 | ATACTTGACATCAGACAGGGGCCGAAAGAACCTTTCCGCGATTATGTGGACAGATTCTACAAAACGCTCAGG |
| | AA | GCCGAACAGGCCAGCCAAGAGGTTAAAAACTGGATGACTGAAACCCTGCTGGTCCAGAATGCTAACCCCGAC |
| | Pol:683-708 | TGCAAGACAATCTTGAAGGCACTTGGGCCGGCCGCAACGTTGGAAGAAATGATGACGGCTTGTCAAGGGGTT |
| | | GGCGGTCCGGGCCATAAGGCCCGAGTCCTGGCAGAAGCTATGAGTCAGAAAGAACCAATAGTTGGAGCCGAA |
| | | ACATTCTACGTCGACGGTGCTGCAAATCGCGAGACGAAGGCTGCTAAGGAAAAGGTCTATCTGGCGTGGGTT |
| | | CCGGCACACAAGGGCATAGGCGGGAATGAGCAAGTCGACAAATTGGTCTCACGAGCTAAACGCGCCCCGGTT |
| | | AAACAAACCCTGAACTTCGACTTGCTTAAACTCGCAGGGGACGTTGAATCAAATCCGGGCCCACTTAGTCCC |
| | | CGAACGCTGAACGCATGGGTAAAAGTAATAGAAGAGAAAGCGTTTTCCCCGGAGGTTATTCCCATGTTTACA |
| | | GCCCTCAGCGAGGGCGCGACGCCACATGATCTCAACACAATGCTTAACACGATTGGGGGGCATCAGGCTGCG |
| | | ATGCAAATGCTCAAGGATACGATAAACGAAGAGGCCGCAGAATGGGACCGAGTACATCCGGTCCACGCCGGG |
| | | CCCGTCGCACCAGGACAGATGCGAGACCCCCGAGGGTCAGACATCGCCGGTTCTACGTCAACCTTGCAAGAA |
| | | CAAATTGCATGGATGACTAACAATCCACCTATCCCCGTGGGAGATATCTATAAGAGATGGATCATCATGGGG |
| | | CTTAATAAAATAGTCAGGATGTATTCACCAGTTTCAATTCTTGATATTAAACAGGGTCCTAAGGAGCCCTTT |
| | | CGAGATTATGTGGATAGGTTTTATAGAACCCTTCGCGCAGAACAGGCTTCACAAGACGTCAAAAATTGGATG |
| | | ACAGAAACTCTTCTCGTACAGAATTCAAACCCGGATTGTAAGACGATCTTGAAAGCACTCGGTCCGGGTGCC |
| | | ACGTTGGAAGAGATGATGTCAGCCTGCCAAGGGGTGGGTGGCCCAAGCCATAAGGCCAGGGTATTGGCAGAG |
| | | GCAATGTGTCAAGCTGTCAAGGCAGCATGCTGGTGGGCTGGCGTAAAGCAGGAATTCGGCATACCGTACAAT |
| | | ACACAGTCACAAGGTGTTGTCGAGAGTATGAATAATGAACTTAAAAAAATCATAGGACAGATCCGAGATCAA |
| | | GCTGAACATCTGAAGACTGCTGTACAAATGGCAGTGCTTATACATAACTTCAAACGCAAGGGAGGAATTGGG |
| | | GAGTATTCAGCGGGAGAGCGGATTATTGATATCATAGCAACCGAACCCATAGCGGGCGTCGAGACTTTCTAT |
| | | GTGGACGGAGCCAGCAATAGGGAGACTAAGGCGGCGAAAGAAAAGATTTATCTGGCATGGGTTCCGGCTCAT |
| | | AAGGGGATCGGAGGCAATGAGCAGATTGACAAACTGGTATCC |
| 211 | Pol:932-1003 | TTGAGCCCCCGAACCCTTAACGCCTGGGTGAAGGTAATTGAGGAAAAAGCTTTTTCACCCGAAGTGATCCCG |
| | AAA | ATGTTTACAGCACTGTCTGAAGGTGCAACCCCACACGATCTCAACACTATGCTCAATACCATAGGGGGCCAC |
| | I | CAAGCAGCGATGCAGATGCTCAAAGATACCATTAACGAGGAAGCGGCCGAGTGGGACAGGGTACACCCCGTG |
| | Pol:129-320 | CATGCCGGACCTGTCGCCCCGGGTCAAATGCGAGATCCACGCGGAAGTGATATCGCTGGGAGTACGTCCACC |
| | Pol:747-827 | CTCCAAGAACAAATCGCGTGGATGACAAATAATCCTCCTATCCCCGTAGGAGATATTTATAAAAGGTGGATT |
| | QEE | ATCATGGGGCTTAATAAAATCGTTCGAATGTATAGTCCTGTCTCAATACTGGACATCAAACAAGGCCCAAAA |
| | Nef:64-76 | GAACCATTCAGGGACTACGTTGACAGATTCTATCGCACACTCCGAGCGGAACAAGCAAGCCAGGACGTCAAA |
| | RAKR | AATTGGATGACCGAGACGTTGCTTGTACAAAATAGCAACCCCGACTGTAAAACGATACTCAAAGCCTTGGGG |
| | F2A linker | CCAGGCGCGACCTTGGAGGAGATGATGTCCGCTTGTCAGGGAGTAGGCGGACCATCACACAAAGCACGCGTT |
| | Pol:747-827 | TTGGCGGAAGCTATGTGTCAGGCGGTAAAGGCAGCCTGTTGGTGGGCTGGAGTCAAACAAGAATTTGGCATC |
| | Pol:932-1003 | CCCTATAATACACAGTCCCAGGGTGTCGTCGAGTCTATGAATAATGAGCTGAAGAAAATCATTGGGCAGATA |
| | Pol:129-320 | AGAGACCAAGCCGAACATCTTAAAACGGCTGTCCAAATGGCGGTTTTGATCCATAATTTTAAGCGAAAAGGG |
| | KIL | GGGATTGGTGAGTACTCAGCAGGAGAGAGAATCATAGACATCATAGCAACAGAACCAATTGCTGGTGTCGAG |
| | QEE | ACTTTTTACGTAGATGGGGCGAGCAATAGGGAAACTAAAGCAGCGAAGGAAAAGATTTACCTCGCGTGGGTA |
| | Nef:64-76 | CCGGCCCACAAGGGAATCGGCGGGAACGAGCAAATCGATAAACTTGTATCCAGAGCCAAACGGGCTCCAGTA |
| | | AAACAGACACTCAATTTCGATCTTTTGAAGCTTGCTGGAGACGTTGAGAGCAATCCTGGGCCGGTAGCAAAG |
| | | GAGATTGTAGCTTGTTGCGACAAGTGCCAGTTGAAGGGTGAAGCGATACACGGTCAGGTCGATTGCTCTCCG |
| | | GGAGTTTGGCAACTTGACTGTACCCATCTCGAGGGCAAAGTTATCCTCGTAGCTGTGCATGTAGCATCAGGA |
| | | TATATAGAGGCCGAGATCATTCCGACGGAAACGGGTCAAGAAACTGCTTACTTCATTCTCAAACTTGCCGGG |
| | | CGGTGGCCAGTCACAACTATCACGAAACTCCAAAACTTTCGAGTTTACTATAGGGACAATCGAGACCCACTG |
| | | TGGAAAGGACCTGCCAGGCTTCTGTGGAAAGGGGAGGGTGCCGTTGTCATACAGGATAACTCCGAGATAAAA |
| | | GTTGTGCCAAGGCGAAAAGTTAAGATTATTCGGGATTACGGGAAACGCATGGCAGGGGATGACTGCGTTGCG |
| | | GGGCGACAAGATGAGGATGGTACTGTACTTATTGGCCCAACACCCGTGAACATTATAGGACGGAATCTGCTG |
| | | ACACAGTTGGGGTGTACGCTCAACTTTCCGATAAGTCCGATAGATACGGTTCCGGTAAAGCTGAAGCCCGGC |
| | | ATGGATGGTCCGCGCGTGAAGCAATGGCCACTCACAGAAGAGAAAATCAAAGCTTTGATAGAAATCTGCACC |
| | | GAAATGGAAAAAGAGGGGAAGATCAGCAGGATCGGCCCGGAGAATCCTTACAACACCCCTATTTTCGCGATT |
| | | AAGAAAAAAGATGGTACAAAATGGAGGAAACTCGTTGATTTTCGGGAGCTCAACAAGAAAACGCAAGACTTC |
| | | TGGGAGGTCCAGCTTGGCATACCCCACCCCTCTGGACTTAAAAAGAAAAAAAGCGTAACCGTACTTGATATT |
| | | GGTGACGCGTATTTCTCCGTTCCCTTGGATAAAGAATTTAGGAAGTACACGGCCTTTACTGTCCCCTCCACT |
| | | AACAACGAAACTCCGGGCGTGCGATATCAATATAATGTGCTTCCGATGGGATGGAAAGGCTCACCAGCGATT |
| | | TTTCAATGCAGTATGACCAAGATTCTTCAGGAAGAAGAGGAAGTGGGGTTTCCGGTAAGACCACAGGTGCCC |
| | | CTC |
| 212 | Pol:840-920 | ACGGTTAAGGCGGCGTGTTGGTGGGCGGGGATAAAGCAAGAGTTCGGAATCCCATATAATCCACAATCCCAA |
| | Gag:147-369 | GGTGTGGTGGAAAGCATGAACAAGGAATTGAAGAAGATTATAGGACAAGTCAGAGATCAGGCCGAGCATCTG |
| | Pol:586-606 | AAAACTGCAGTTCAGATGGCTGTGTTCATCCACAATTTTAAACGCAAAGGAGGAATTGGTGGATATAGCGCT |
| | AA | GGCGAGAGGATTGTAGACATTATTGCCATATCACCTCGCACTCTGAACGCCTGGGTGAAGGTTGTTGAAGAA |
| | Pol:683-708 | AAGGCTTTTTCACCGGAGGTAATCCCAATGTTCAGTGCTCTTAGCGAGGGGGCAACTCCGCAGGACCTTAAT |
| | RAKR | ACAATGTTGAACACTGTAGGGGGACATCAAGCCGCTATGCAAATGCTGAAGGAGACGATTAACGAGGAAGCT |
| | F2A linker | GCGGAATGGGATAGACTTCACCCCGTCCACGCTGGACCTATTGCACCGGGACAGATGCGCGAACCAAGAGGT |
| | Pol:932-1003 | TCCGATATAGCGGGAACAACTAGCACACTCCAGGAACAGATAGGATGGATGACCAACAACCCTCCGATACCA |
| | AAA | GTAGGCGAAATCTACAAGCGCTGGATAATATTGGGGCTGAACAAAATCGTCAGGATGTACAGCCCAACTTCA |
| | I | ATATTGGACATTCGCCAAGGACCTAAAGAGCCGTTCCGGGATTACGTGGATAGGTTTTACAAGACTTTGCGA |
| | Pol:129-320 | GCTGAACAAGCCAGTCAAGAGGTGAAAAACTGGATGACCGAGACTCTGCTCGTCCAAAATGCTAATCCAGAT |
| | Pol:747-827 | TGCAAAACAATACTTAAGGCACTGGGTCCCGCCGCAACGCTCGAGGAGATGATGACTGCCTGCCAAGGTGTC |
| | QEE | GGTGGTCCGGGTCACAAAGCACGAGTCCTGGCGGAAGCCATGTCTCAGAAAGAGCCTATAGTGGGTGCCGAG |
| | Nef:64-76 | ACGTTCTACGTTGATGGAGCCGCTAATCGAGAGACGAAAGCGGCCAAGGAAAAGGTGTATCTCGCTTGGGTG |
| | | CCTGCTCATAAGGGCATCGGAGGTAATGAACAAGTTGATAAACTGGTGAGTCGGGCGAAGCGCGCACCAGTA |
| | | AAGCAGACCCTTAATTTCGATTTGCTCAAACTCGCTGGTGATGTCGAATCTAACCCCGGTCCGATTACAAAA |
| | | ATCCAGAATTTTAGGGTTTACTATCGAGATTCCCGAGATCCACTCTGGAAAGGCCCCGCGAAATTGCTCTGG |
| | | AAGGGCGAAGGGGCTGTAGTAATTCAAGACAATTCTGATATCAAGGTAGTCCCTCGGAGGAAAGCTAAAATA |
| | | ATACGAGACTATGGAAAACAGATGGCGGGGGATGACTGTGTAGCAAGCCGGCAAGATGAAGACGCGGCAGCT |
| | | ATAGGAACAGTGCTGGTGGGGCCGACCCCCGTAAACATTATCGGCAGGAATCTGTTGACGCAAATAGGTTGT |
| | | ACGCTCAATTTTCCTATCTCACCGATCGAAACGGTGCCCGTCAAGTTGAAGCCGGGCATGGACGGCCCAAAG |
| | | GTAAAACAATGGCCCTTGACGGAGGAGAAAATCAAAGCTCTTGTCGAAATCTGTACCGAAATGGAAAAGGAA |
| | | GGTAAGATAAGTAAAATCGGACCAGAAAACCCGTATAACACTCCAGTTTTCGCGATAAAGAAGAAAGACTCC |
| | | ACAAAGTGGAGAAAACTTGTAGATTTCAGGGAGCTGAATAAAAGGACCCAGGATTTTTGGGAAGTCCAGTTG |
| | | GGCATACCACATCCCGCGGGGCTCAAAAAGAAGAAGTCAGTCACGGTACTCGACGTTGGCGACGCATATTTC |
| | | TCTGTTCCGCTCGATAAGGACTTCAGAAAATATACCGCTTTCACTATTCCAAGTATCAACAATGAAACTCCC |
| | | GGGATACGCTATCAATACAACGTTCTGCCACAGGGATGGAAGGGGAGTCCGGCTATTTTTCAGTCTTCAATG |
| | | ACAGTGGCAAAAGAGATCGTTGCAAGCTGTGATAAATGCCAACTGAAAGGTGAGGCCATGCACGGACAGGTT |
| | | GACTGCTCTCCCGGGATATGGCAGCTGGATTGCACGCATTTGGAGGGTAAGATAATTCTCGTCGCGGTCCAC |
| | | GTGGCTAGTGGCTACATCGAGGCCGAAGTAATCCCCGCAGAGACGGGCCAAGAAACTGCGTACTTCCTCCTG |
| | | AAGCTGGCAGGACGATGGCCTGTCAAAACACAGGAGGAAGAAGAAGTGGGATTCCCGGTTAAGCCCCAGGTT |
| | | CCGCTG |
| 213 | Gag:147-369 | CTTAGCCCACGGACACTTAATGCATGGGTCAAGGTGATTGAGGAGAAGGCGTTTTCCCCAGAGGTGATCCCA |
| | Pol:840-920 | ATGTTCACAGCCCTCTCTGAGGGCGCAACACCTCACGACCTGAATACAATGCTCAACACTATCGGAGGGCAC |
| | Pol:586-606 | CAAGCAGCCATGCAAATGCTGAAAGACACCATCAACGAGGAGGCTGCTGAGTGGGATCGAGTACACCCTGTA |
| | AA | CACGCGGGGCCGGTTGCTCCTGGTCAAATGAGGGATCCCCGCGGCTCCGACATAGCCGGCTCAACAAGCACT |
| | Pol:683-708 | CTGCAGGAACAGATAGCATGGATGACCAACAACCCCCCTATTCCCGTAGGGGACATTTACAAGAGGTGGATT |
| | RAKR | ATAATGGGTCTGAACAAGATCGTACGAATGTATTCACCGGTCAGCATACTTGATATAAAACAAGGCCCGAAA |
| | F2A linker | GAACCTTTCAGGGACTATGTCGATCGATTTTACCGCACGCTGCGCGCCGAGCAGGCCTCCCAGGACGTGAAG |
| | Pol:747-827 | AACTGGATGACTGAAACACTGCTTGTGCAAAATTCAAATCCAGATTGTAAAACCATACTTAAGGCTCTTGGT |
| | Pol:932-1003 | CCGGGGGCTACCCTGGAAGAGATGATGAGCGCGTGTCAAGGGGTAGGAGGTCCTTCTCACAAGGCCAGGGTT |
| | Pol:129-320 | TTGGCTGAAGCTATGTGCCAGGCCGTTAAGGCGGCCTGTTGGTGGGCAGGAGTCAAGCAAGAATTCGGCATC |
| | KIL | CCCTACAACACGCAATCACAGGGTGTCGTTGAATCAATGAACAACGAGCTGAAAAAAATTATAGGTCAAATA |
| | QEE | CGCGATCAAGCGGAACATCTTAAGACCGCAGTGCAGATGGCTGTGCTTATACATAATTTCAAGCGAAAAGGA |
| | Nef:64-76 | GGCATCGGAGAATACTCAGCCGGAGAAAGAATCATCGACATTATCGCAACGGAGCCAATCGCCGGTGTGGAA |
| | | ACGTTCTATGTAGACGGAGCATCAAATAGGGAAACGAAGGCCGCCAAGGAGAAAATTTATCTGGCGTGGGTC |
| | | CCGGCCCACAAGGGAATTGGCGGCAATGAACAGATCGACAAGCTTGTTTCTCGGGCTAAACGGGCTCCCGTT |
| | | AAGCAGACGCTCAACTTCGATCTTCTCAAGCTGGCCGGTGATGTCGAGAGCAATCCGGGGCCGGTAGCTAAG |
| | | GAAATCGTCGCTTGTTGTGATAAATGTCAACTTAAGGGTGAAGCAATTCATGGCCAAGTTGACTGTTCACCG |
| | | GGAGTATGGCAACTCGATTGTACCCATCTCGAAGGAAAAGTCATCTTGGTTGCAGTGCACGTAGCTTCTGGC |
| | | TACATTGAAGCGGAGATTATCCCGACAGAGACCGGGCAGGAGACCGCTTATTTTATCCTCAAGCTCGCAGGA |
| | | CGATGGCCCGTCACTACTATCACGAAGCTCCAAAATTTTAGAGTGTACTACCGCGATAACAGAGATCCTTTG |
| | | TGGAAAGGCCCCGCCAGACTTTTGTGGAAGGGTGAGGGAGCGGTGGTTATTCAGGACAATTCTGAGATAAAA |
| | | GTTGTACCCCGACGGAAGGTCAAGATAATTAGAGATTATGGTAAAAGGATGGCGGGCGACGACTGTGTAGCT |
| | | GGAAGGCAGGATGAGGACGGCACAGTGCTGATTGGCCCGACGCCCGTAAACATTATCGGTCGCAACCTTCTC |
| | | ACCCAGCTGGGGTGCACTTTGAATTTCCCGATTTCCCCGATTGATACTGTTCCAGTAAAGCTCAAACCCGGG |
| | | ATGGACGGCCCACGAGTAAAACAATGGCCATTGACAGAGGAGAAGATTAAGGCGCTTATCGAAATATGTACT |
| | | GAAATGGAGAAGGAAGGGAAAATTAGTCGGATAGGGCCTGAGAATCCCTACAACACGCCCATTTTTGCTATC |
| | | AAGAAGAAAGATGGCACCAAGTGGCGGAAGCTGGTCGATTTTCGGGAACTTAACAAGAAAACACAAGACTTC |
| | | TGGGAAGTACAGCTTGGGATCCCGCACCCGTCAGGATTGAAGAAGAAAAAGAGCGTCACGGTACTCGACATA |
| | | GGCGACGCTTACTTCTCAGTTCCGCTGGACAAAGAGTTCAGAAAATATACAGCTTTCACGGTACCCTCCACT |
| | | AACAATGAGACACCTGGAGTTCGCTACCAGTACAATGTGCTTCCAATGGGATGGAAGGGCTCACCGGCTATT |
| | | TTCCAATGCTCTATGACTAAAATACTTCAAGAGGAAGAAGAGGTTGGGTTTCCCGTCAGACCGCAGGTTCCA |
| | | CTT |
| 214 | Pol:840-920 | ACGGTGAAAGCAGCTTGCTGGTGGGCGGGGATCAAACAGGAGTTTGGAATACCTTATAATCCTCAATCACAG |
| | Gag:147-369 | GGGGTTGTCGAAAGCATGAACAAGGAGCTCAAGAAGATCATCGGACAGGTGCGCGATCAGGCTGAACATCTT |
| | Pol:586-606 | AAGACCGCAGTTCAGATGGCAGTCTTTATTCACAATTTTAAACGGAAAGGAGGTATAGGTGGCTACAGCGCG |
| | Pol:747-827 | GGCGAGCGCATTGTAGATATTATAGCGATTTCTCCGCGGACGCTGAATGCATGGGTTAAAGTAGTTGAAGAG |
| | Pol:683-708 | AAGGCCTTTTCTCCCGAAGTAATACCCATGTTCAGTGCACTGTCTGAAGGTGCTACTCCTCAGGATCTCAAC |
| | Nef:64-76 | ACGATGCTCAACACGGTCGGTGGGCATCAGGCAGCAATGCAGATGCTGAAGGAAACGATAAACGAGGAGGCA |
| | Pol:932-1003 | GCAGAATGGGATCGACTGCACCCAGTACACGCAGGCCCTATAGCCCCAGGTCAAATGCGGGAACCAAGAGGT |
| | AAA | AGTGATATAGCTGGGACTACCTCAACGTTGCAGGAGCAAATTGGTTGGATGACGAATAATCCTCCTATACCA |
| | Pol:129-320 | GTTGGCGAAATATACAAAAGATGGATTATCTTGGGACTGAATAAAATCGTGCGAATGTATTCTCCGACCTCT |
| | | ATACTGGACATTCGACAGGGACCAAAAGAGCCGTTCCGCGACTACGTCGATCGGTTTTATAAAACTTTGCGG |
| | | GCCGAACAGGCAAGCCAGGAGGTAAAGAACTGGATGACAGAGACCCTGTTGGTGCAAAATGCGAACCCTGAT |
| | | TGCAAGACCATACTGAAAGCACTCGGGCCAGCTGCCACCCTTGAGGAAATGATGACAGCTTGCCAGGGTGTG |
| | | GGGGGGCCGGGGCATAAAGCACGCGTCCTCGCCGAAGCCATGTCACAGAAAGAACCAATTGTGGGTGCCGAA |
| | | ACTTTTTACGTGGACGGCGCAGCCAACCGAGAGACTAAAGTGGCTAAAGAGATAGTTGCATCATGTGATAAG |
| | | TGCCAATTGAAAGGTGAGGCCATGCACGGTCAGGTAGATTGTTCACCTGGTATATGGCAGTTGGACTGTACT |
| | | CACCTTGAAGGAAAGATTATCCTGGTCGCGGTACACGTCGCATCCGGTTATATAGAGGCGGAAGTTATACCT |
| | | GCGGAGACTGGTCAAGAAACTGCCTACTTCCTTCTTAAATTGGCTGGTCGATGGCCAGTAAAAACTAAAGAG |
| | | AAAGTGTACCTTGCGTGGGTTCCAGCCCACAAGGGTATAGGAGGAAATGAGCAAGTAGACAAACTCGTAAGC |
| | | CAAGAGGAAGAAGAAGTGGGTTTCCCAGTTAAGCCACAGGTACCCCTCATTACCAAAATACAGAATTTCCGG |
| | | GTTTATTATCGCGATTCAAGGGACCCCCTGTGGAAAGGTCCAGCAAAACTGCTGTGGAAGGGCGAAGGGGCA |
| | | GTTGTTATACAAGACAACTCAGATATCAAGGTCGTGCCAAGACGCAAAGCTAAAATTATAAGGGATTATGGT |
| | | AAACAGATGGCTGGAGACGACTGCGTGGCCAGCAGACAAGACGAGGATGCAGCTGCAATTGGAACAGTCCTG |
| | | GTCGGACCAACTCCCGTTAACATCATAGGTAGAAACTTGCTCACTCAAATCGGATGCACACTTAATTTTCCG |
| | | ATTTCACCTATCGAGACCGTTCCCGTTAAGCTGAAACCTGGGATGGACGGTCCCAAGGTGAAGCAATGGCCC |
| | | TTGACTGAGGAAAAGATAAAGGCGTTGGTAGAGATCTGCACCGAAATGGAGAAGGAAGGCAAGATATCTAAG |
| | | ATCGGGCCAGAAAACCCATATAATACACCTGTCTTCGCGATAAAAAAAAAGGACTCTACTAAATGGAGAAAA |
| | | CTGGTCGACTTCAGAGAGCTTAATAAGCGAACTCAAGACTTTTGGGAAGTGCAGCTTGGTATACCTCACCCT |
| | | GCTGGTCTGAAGAAGAAAAAATCTGTTACTGTTCTTGATGTCGGTGACGCATACTTCAGTGTGCCCCTCGAT |
| | | AAAGATTTCAGGAAATACACCGCGTTCACTATACCCAGCATTAATAACGAGACCCCCGGGATACGCTACCAA |
| | | TACAATGTCCTCCCCCAGGGCTGGAAAGGGTCTCCAGCAATTTTTCAGTCATCAATGACG |
| 215 | Gag:147-369 | TTGTCCCCTCGGACGCTCAATGCATGGGTTAAAGTTATCGAGGAGAAGGCCTTCAGTCCCGAGGTTATACCT |
| | Pol:747-827 | ATGTTCACCGCTCTGTCTGAAGGAGCAACGCCCCATGATCTCAATACTATGCTCAATACAATTGGAGGTCAC |
| | Pol:683-708 | CAAGCGGCTATGCAAATGCTCAAAGATACCATTAATGAGGAGGCTGCTGAATGGGATAGGGTCCACCCAGTT |
| | Pol:586-606 | CATGCCGGACCGGTGGCTCCGGGACAGATGCGCGACCCCCGGGGGTCAGACATCGCCGGAAGTACCTCTACT |
| | Pol:932-1003 | CTGCAGGAACAAATTGCATGGATGACAAATAATCCACCTATTCCGGTCGGAGACATCTACAAACGATGGATC |
| | Pol:840-920 | ATAATGGGTCTCAACAAGATAGTCCGGATGTATAGTCCGGTAAGTATACTCGACATCAAGCAAGGCCCTAAG |
| | Nef:64-76 | GAGCCGTTCCGGGATTACGTAGACCGATTCTACCGGACGCTCAGAGCCGAACAGGCCTCCCAAGATGTTAAG |
| | Pol:129-320 | AACTGGATGACCGAAACGTTGTTGGTTCAAAATTCCAATCCTGATTGCAAAACGATACTCAAAGCTCTTGGT |
| | | CCTGGTGCAACACTGGAGGAAATGATGTCAGCCTGCCAAGGGGTCGGCGGGCCTTCACACAAAGCAAGGGTT |
| | | TTGGCGGAGGCAATGTGCCAAGTAGCGAAGGAAATAGTGGCCTGTTGTGACAAATGTCAGCTGAAAGGAGAG |
| | | GCAATACATGGACAAGTTGACTGTTCTCCCGGTGTGTGGCAACTCGACTGTACCCACTTGGAAGGAAAAGTT |
| | | ATACTGGTGGCCGTTCACGTCGCGTCTGGCTACATCGAGGCTGAGATCATACCTACAGAGACCGGGCAGGAG |
| | | ACCGCGTACTTCATCCTTAAGCTCGCGGGCCGCTGGCCAGTCACGACTAAAGAGAAAATTTATCTGGCGTGG |
| | | GTACCCGCGCATAAAGGTATTGGCGGCAATGAACAAATAGACAAATTGGTATCAACAGAGCCGATTGCAGGA |
| | | GTCGAAACATTCTATGTTGATGGTGCGTCAAACAGGGAAACGAAGATAACAAAGTTGCAAAACTTTCGAGTC |
| | | TACTATCGCGACAATCGGGATCCCCTCTGGAAGGGCCCAGCAAGGTTGCTGTGGAAGGGCGAGGGAGCAGTA |
| | | GTCATTCAAGACAACAGTGAGATTAAGGTAGTTCCGCGACGGAAGGTCAAAATAATACGGGATTACGGCAAA |
| | | AGGATGGCAGGGGATGATTGCGTGGCTGGGCGCCAGGATGAGGACGCTGTCAAAGCCGCGTGTTGGTGGGCA |
| | | GGGGTTAAGCAGGAGTTCGGAATACCATACAACACCCAGTCTCAAGGAGTTGTTGAAAGCATGAACAATGAG |
| | | CTTAAAAAAATAATCGGACAAATAAGGGATCAGGCCGAACACTTGAAGACAGCAGTTCAGATGGCCGTGCTG |
| | | ATCCATAACTTCAAACGGAAGGGCGGCATAGGAGAATACTCCGCAGGCGAGAGAATAATCGACATTATAGCT |
| | | CAAGAAGAAGAGGAAGTCGGCTTTCCTGTACGACCTCAGGTCCCTCTTGGTACGGTGTTGATAGGGCCGACC |
| | | CCCGTCAACATCATCGGTCGGAACCTGCTTACACAACTTGGTTGCACTCTTAACTTTCCTATTTCCCCCATA |
| | | GATACCGTCCCAGTCAAGTTGAAGCCGGGGATGGATGGCCCGCGCGTCAAGCAGTGGCCCCTGACTGAAGAA |
| | | AAGATTAAAGCTCTGATTGAAATATGCACAGAAATGGAAAAAGAGGGTAAGATCAGCAGAATCGGTCCAGAA |
| | | AATCCCTATAACACGCCGATATTCGCCATTAAGAAGAAGGACGGAACAAAGTGGCGGAAACTCGTCGATTTT |
| | | AGGGAGCTGAATAAGAAAACGCAGGATTTCTGGGAAGTTCAACTTGGCATACCTCACCCCTCTGGTCTTAAA |
| | | AAAAAAAAGTCAGTCACCGTTCTCGACATTGGGGACGCGTATTTTTCCGTTCCGCTCGACAAAGAGTTTCGG |
| | | AAGTACACGGCGTTCACGGTACCTTCTACAAACAATGAAACCCCCGGGGTCAGGTATCAGTATAATGTGCTG |
| | | CCAATGGGGTGGAAGGGTAGCCCTGCTATTTTTCAATGCTCAATGACAAAGATCCTT |
| 216 | M | ATGACAGTAAAAGCGGCTTGCTGGTGGGCGGGTATTAAGCAAGAGTTTGGTATCCCCTACAACCCCCAGTCC |
| | Pol:840-920 | CAAGGAGTCGTCGAGTCTATGAACAAAGAACTGAAGAAGATCATTGGGCAGGTGCGCGATCAGGCTGAGCAC |
| | Gag:147-369 | TTGAAAACAGCGGTCCAGATGGCTGTGTTTATCCATAACTTTAAGAGGAAGGGGGGGATAGGGGGCTATTCA |
| | Gag:1-53 | GCAGGGGAGAGAATTGTAGACATCATCGCCATATCCCCTCGAACGCTCAATGCGTGGGTTAAAGTAGTTGAG |
| | PPV | GAAAAGGCATTTAGTCCTGAAGTCATCCCAATGTTTAGCGCACTTTCCGAGGGCGCTACGCCCCAGGACCTG |
| | Pol:747-827 | AATACCATGCTTAACACCGTTGGGGGCCACCAGGCGGCCATGCAGATGCTCAAGGAAACTATTAACGAGGAG |
| | Pol:683-708 | GCGGCGGAGTGGGATCGGCTGCACCCTGTCCACGCAGGACCGATCGCCCCGGGGCAAATGAGAGAACCCAGA |
| | Nef:117-148 | GGTTCTGATATTGCTGGAACTACTAGTACTCTTCAGGAGCAAATCGGGTGGATGACTAATAACCCACCAATT |
| | RAKR | CCCGTAGGTGAAATTTACAAGAGATGGATCATACTGGGCTTGAACAAAATAGTCCGAATGTATAGTCCCACC |
| | F2A linker | TCAATCCTCGACATCCGGCAAGGACCGAAGGAGCCTTTCCGCGACTATGTGGATCGCTTTTATAAGACTCTG |
| | M | CGAGCAGAACAAGCATCACAAGAGGTTAAAAACTGGATGACCGAAACACTCTTGGTGCAGAACGCAAATCCC |
| | Gag:147-369 | GACTGCAAAACCATCCTGAAGGCATTGGGCCCTGCAGCAACTTTGGAGGAGATGATGACTGCATGTCAAGGC |
| | Pol:747-827 | GTAGGAGGGCCCGGCCATAAAGCCAGAGTTTTGGCAGAGGCTATGTCTCAAATGGCGGCACGAGCTTCAGTT |
| | Gag:1-53 | CTGTCAGGGGGCGAACTTGATCGGTGGGAAAAGATACGGCTTCGGCCCGGAGGCAAGAAAAAGTACAGGCTG |
| | AA | AAGCACATAGTATGGGCGTCCCGCGAACTGGAGAGGTTTGCAGTGAACCCCGGCCTGCTCGAGACGCCCCCG |
| | Pol:840-920 | GTGGTTGCTAAAGAAATAGTCGCCTCTTGTGATAAATGCCAACTCAAGGGAGAAGCTATGCATGGCCAGGTT |
| | Nef:117-148 | GACTGCTCACCGGGTATATGGCAGCTGGATTGTACACATTTGGAAGGTAAAATCATACTCGTTGCTGTGCAT |
| | LIK | GTAGCAAGCGGGTATATTGAGGCGGAAGTAATTCCGGCGGAAACCGGGCAAGAAACTGCCTATTTCCTTCTT |
| | Pol:683-708 | AAACTCGCGGGGCGGTGGCCGGTTAAGACCAAGGAGAAAGTCTATCTCGCATGGGTTCCGGCCCATAAAGGC |
| | | ATCGGCGGTAATGAACAAGTAGATAAACTCGTTAGCACTCAAGGATATTTTCCGGATTGGCAGAATTATACA |
| | | CCCGGACCTGGTACAAGATATCCCTTGACGTTCGGATGGTGTTTCAAGCTCGTCCCAGTCCGCGCTAAAAGA |
| | | GCACCAGTAAAGCAGACCTTGAACTTCGACTTGCTCAAGCTTGCTGGGGATGTCGAAAGTAACCCCGGCCCG |
| | | ATGTTGTCCCCCAGGACTTTGAATGCATGGGTCAAAGTGATTGAGGAGAAGGCCTTCTCCCCCGAAGTTATT |
| | | CCGATGTTTACCGCGCTTAGTGAAGGGGCCACACCTCATGATCTGAATACGATGCTTAACACTATAGGGGGT |
| | | CACCAGGCAGCGATGCAAATGCTGAAGGATACCATCAATGAAGAAGCAGCTGAATGGGACAGGGTACATCCA |
| | | GTGCATGCAGGACCGGTTGCACCCGGACAAATGCGCGACCCGCGAGGTTCCGACATCGCGGGGTCAACGTCC |
| | | ACCCTGCAAGAACAAATTGCATGGATGACCAATAATCCCCCTATCCCAGTGGGCGACATATATAAGCGCTGG |
| | | ATAATCATGGGTCTCAATAAAATTGTAAGGATGTATAGTCCGGTGTCAATCCTGGACATAAAGCAAGGTCCC |
| | | AAGGAACCGTTTCGCGACTATGTAGACAGATTTTATCGAACGCTGAGAGCCGAGCAAGCGAGCCAGGATGTC |
| | | AAAAACTGGATGACCGAAACACTTCTCGTTCAGAATTCAAACCCGGATTGTAAGACAATACTTAAGGCGCTC |
| | | GGTCCCGGGGCGACCCTTGAAGAGATGATGTCTGCTTGTCAAGGTGTTGGGGGTCCATCCCACAAAGCTCGC |
| | | GTCCTGGCGGAAGCAATGTGCCAAGTCGCCAAAGAAATCGTCGCGTGCTGTGACAAGTGCCAACTCAAGGGT |
| | | GAGGCGATCCATGGGCAAGTGGACTGTAGTCCAGGCGTATGGCAATTGGACTGTACGCATCTCGAAGGGAAG |
| | | GTGATCTTGGTGGCCGTCCATGTGGCGAGCGGATATATTGAAGCCGAAATCATCCCTACCGAAACGGGACAA |
| | | GAAACGGCGTATTTCATTTTGAAACTGGCGGGTCGGTGGCCGGTCACCACCATGGCCGCGCGAGCGAGCATA |
| | | CTTAGCGGGGGTAAATTGGACAAGTGGGAGAAGATCCGGCTTCGGCCCGGGGGTCGGAAAAAATATAAGCTG |
| | | AAGCACCTGGTCTGGGCATCACGAGAACTGGAGCGGTTCGCACTTAATCCAGGGTTGCTTGAAACCGCAGCC |
| | | GCGGTGAAGGCCGCCTGCTGGTGGGCGGGAGTAAAGCAGGAGTTCGGAATTCCTTACAACACGCAGAGCCAA |
| | | GGTGTAGTGGAAAGCATGAACAATGAGCTTAAGAAAATCATTGGTCAGATCAGAGACCAAGCGGAACACCTC |
| | | AAGACAGCTGTGCAAATGGCTGTACTTATTCACAACTTCAAGAGAAAAGGCGGTATAGGAGAATATAGCGCG |
| | | GGGGAAAGAATAATAGACATCATCGCTACTCAAGGGTTTTTCCCCGACTGGCAGAATTATACACCTGGCCCC |
| | | GGTATACGGTTTCCACTTACTTTCGGCTGGTGTTTCAAGTTGGTGCCTCTCCTGATAAAAAAGGAGAAAATA |
| | | TATCTTGCGTGGGTGCCTGCACACAAAGGTATAGGCGGTAACGAACAAATCGACAAATTGGTTAGC |
| 217 | M | ATGTTCCCCCAGATTACACTGTGGCAGCGACCTCTGGTCACCATCAAGATTGGGGGACAGCTTAAGGAAGCG |
| | Pol:56-117 | CTGCTTGACACTGGTGCTGATGACACTGTACTGGAGGAGATGAATCTCCCGGGTCGCTGGAAGCCTAAAATG |
| | Nef:64-99 | ATAGGCGGTATCGGGGGGTTCATTAAGGTCAGGCAGTACGACCAAGAAGAGGTCGGATTCCCGGTAAAGCCA |
| | LI | CAAGTGCCTCTTCGCCCGATGACGTTCAAGGGTGCGTTGGACCTCAGCCACTTTCTTCGAGAAAAGGGCGGA |
| | Pol:542-606 | CTGGAAGGTCTGATACCTAAATTTAAGCTCCCTATTCAGAAGGAAACATGGGAGACATGGTGGACGGAGTAT |
| | Pol:367-431 | TGGCAGGCGACATGGATCCCCGAGTGGGAATTTGTAAACACCCCGCCACTCGTAAAACTCTGGTACCAACTG |
| | Pol:932-1003 | GAAAAGGAACCCATCGTCGGTGCTGAAACCTTCTACGTCGATGGTGCGGCTAATAGGGAAACGAAGTGGGGT |
| | Pol:129-320 | TTCACTACCCCAGACAAAAAACATCAAAAAGAACCACCCTTTCTCTGGATGGGATATGAATTGCATCCCGAT |
| | RAKR | AAATGGACCGTGCAACCCATCGTTCTGCCGGAAAAGGACAGCTGGACCGTTAATGATATTCAGAAGCTTGTT |
| | F2A linker | GGGAAACTCAACTGGGCTTCCCAAATTTACCCGGGAATAAAGGTGATAACGAAAATTCAGAATTTTAGGGTG |
| | M_ | TACTATAGGGACTCACGCGATCCTCTTTGGAAAGGTCCAGCAAAGTTGTTGTGGAAAGGTGAGGGGGCTGTC |
| | Pol:56-117 | GTCATCCAAGACAATAGTGATATTAAGGTCGTGCCTAGAAGAAAGGCAAAGATTATTAGGGATTACGGCAAG |
| | Pol:367-431 | CAGATGGCTGGTGACGACTGTGTTGCAAGTCGCCAAGACGAAGATGGCACGGTGTTGGTCGGGCCCACACCA |
| | AA | GTAAACATTATAGGCCGAAATCTGCTTACTCAAATCGGATGTACTCTTAATTTTCCGATCTCCCCTATAGAA |
| | QEE | ACGGTTCCTGTAAAATTGAAACCTGGAATGGATGGTCCGAAAGTTAAACAGTGGCCGCTCACCGAGGAAAAG |
| | Nef:64-99 | ATTAAAGCGCTTGTCGAGATCTGTACTGAAATGGAAAAAGAAGGAAAGATCTCCAAAATAGGGCCAGAAAAT |
| | Pol:932-1003 | CCGTACAATACTCCAGTCTTTGCTATAAAGAAGAAGGATTCTACGAAGTGGAGGAAGCTGGTAGACTTTCGC |
| | Pol:542-606 | GAGCTCAACAAACGCACGCAAGATTTTTGGGAAGTCCAGTTGGGCATCCCTCATCCAGCTGGACTCAAGAAA |
| | AA | AAAAAATCCGTCACAGTATTGGATGTGGGCGACGCCTACTTTTCAGTGCCATTGGACAAAGATTTTCGAAAA |
| | Pol:129-320 | TACACCGCGTTCACAATTCCTAGTATCAATAACGAGACTCCCGGAATAAGGTACCAGTACAACGTGCTCCCT |
| | | CAAGGGTGGAAAGGTTCTCCCGCGATATTTCAGTCCAGTATGACTCGCGCGAAACGAGCTCCAGTTAAACAG |
| | | ACCCTCAACTTTGATTTGTTGAAGCTTGCTGGGGATGTTGAGAGTAATCCAGGCCCTATGCTGCCGCAAATC |
| | | ACACTCTGGCAAAGGCCGATAGTGACCATTAAAATTGGCGGGCAGATCAAGGAGGCATTGCTTGATACGGGA |
| | | GCAGACGATACAGTGTTGGAGGACATGAACCTGCCCGGAAAATGGAAACCAAAGATGATCGGTGGGATTGGC |
| | | GGTTTCATAAAGGTCAAGCAGTATGACCAGTGGGGTCTGACAACCCCTGACAAAAAACATCAGAAGGATCCC |
| | | CCCTTTCTTTGGATGGGTTATGAGTTGCATCCAGATCGCTGGACGGTGCAGCCTATTGAGCTTCCGGAAAAG |
| | | GAGTCTTGGACAGTTAATGATATTCAAAAACTTATTGGGAAATTGAATTGGGCCAGCCAGATATACGCAGGT |
| | | ATAAAAGTTGCGGCGCAGGAGGAGGAAGAAGTGGGGTTCCCCGTCCGACCCCAGGTGCCGCTCAGACCAATG |
| | | ACGTATAAAGGTGCGTTGGATCTGAGTCATTTTTTGAAGGAAAAAGGCGGGTTGGAAGGCATTACCAAACTC |
| | | CAAAACTTCCGGGTGTATTATCGGGACAACAGAGATCCACTCTGGAAGGGTCCCGCAAGATTGCTTTGGAAG |
| | | GGAGAAGGAGCAGTTGTTATACAAGACAACTCCGAAATTAAGGTAGTGCCTAGACGGAAGGTTAAAATTATT |
| | | AGGGACTACGGAAAACGGATGGCGGGGGATGACTGCGTCGCGGGCCGCCAGGACGAGGACCCGAAATTCCGC |
| | | CTGCCTATACAAAAGGAGACGTGGGACACGTGGTGGACAGACTACTGGCAAGCAACGTGGATCCCGGAATGG |
| | | GAATTTACTAACACACCTCCTTTGGTGAAACTCTGGTATCAACTCGAGACGGAGCCGATTGCAGGGGTCGAG |
| | | ACATTTTACGTCGATGGAGCATCCAATAGGGAAACTAAAGCAGCTGGTACAGTTCTGATAGGTCCGACCCCG |
| | | GTGAATATAATAGGCAGGAATCTCCTCACACAACTTGGCTGCACTTTGAATTTCCCAATTTCCCCAATTGAC |
| | | ACCGTACCCGTAAAGTTGAAGCCTGGAATGGACGGACCACGAGTGAAGCAGTGGCCTCTCACGGAAGAAAAG |
| | | ATCAAAGCGCTTATTGAAATTTGTACAGAAATGGAGAAGGAGGGTAAAATCTCCAGGATAGGTCCTGAAAAC |
| | | CCGTACAACACGCCCATCTTCGCTATCAAAAAAAAAGATGGAACGAAATGGCGCAAGCTGGTGGACTTCAGA |
| | | GAACTTAACAAAAAGACGCAGGATTTTTGGGAAGTCCAGTTGGGAATCCCTCACCCGAGCGGACTTAAAAAA |
| | | AAGAAAAGTGTCACAGTTCTTGATATAGGCGACGCTTATTTTTCCGTCCCACTTGACAAGGAATTTAGGAAG |
| | | TACACGGCGTTTACAGTGCCATCAACGAACAACGAAACCCCGGGGGTGCGCTACCAGTACAACGTACTGCCA |
| | | ATGGGATGGAAAGGTTCACCCGCAATCTTTCAATGCTCAATGACT |
| 218 | M | ATGACGGTAAAGGCAGCATGCTGGTGGGCAGGTATAAAACAGGAATTCGGCATTCCGTATAACCCACAAAGT |
| | Pol:840-920 | CAAGGAGTTGTCGAGTCCATGAACAAAGAATTGAAAAAGATAATTGGTCAAGTGCGAGACCAAGCAGAACAC |
| | Gag:147-369 | CTGAAAACCGCGGTTCAAATGGCCGTGTTTATACACAACTTTAAGAGAAAAGGGGGCATCGGGGGCTACTCC |
| | Gag:1-53 | GCGGGTGAACGCATAGTCGATATAATAGCCATCTCCCCTCGCACTCTCAACGCATGGGTGAAAGTCGTAGAG |
| | PPV | GAGAAAGCTTTCTCACCTGAAGTAATTCCGATGTTTAGTGCACTGAGTGAAGGCGCTACGCCTCAAGATCTG |
| | Pol:747-827 | AACACGATGCTTAATACCGTCGGGGGTCACCAAGCCGCGATGCAGATGTTGAAGGAAACAATAAATGAGGAA |
| | Pol:683-708 | GCAGCAGAGTGGGACAGACTTCACCCGGTCCACGCGGGACCAATCGCACCAGGACAAATGCGAGAACCGAGA |
| | Nef:117-148 | GGTAGTGACATCGCCGGAACAACCTCCACCCTCCAGGAACAGATTGGTTGGATGACAAATAATCCTCCGATA |
| | RAKR | CCCGTCGGTGAGATCTACAAACGCTGGATCATCCTGGGTCTTAACAAGATCGTACGGATGTACAGCCCAACC |
| | F2A linker | AGTATCCTTGACATTAGGCAGGGACCGAAGGAGCCGTTTCGCGACTACGTCGATCGGTTTTACAAGACGCTT |
| | M | AGAGCGGAACAAGCGTCACAGGAAGTTAAAAATTGGATGACAGAAACCTTGCTTGTCCAGAATGCTAATCCC |
| | Pol:56-117 | GATTGCAAAACTATTCTGAAGGCACTGGGTCCTGCGGCGACTTTGGAGGAGATGATGACGGCCTGTCAAGGT |
| | Nef:64-99 | GTTGGAGGCCCTGGTCATAAGGCACGAGTCCTGGCTGAAGCAATGTCTCAAATGGCGGCTAGAGCCTCTGTG |
| | LI | CTGTCCGGAGGGGAGCTTGACCGCTGGGAAAAGATCCGATTGCGACCAGGTGGGAAAAAGAAGTACAGGCTC |
| | Pol:542-606 | AAGCATATTGTGTGGGCATCACGGGAACTTGAGCGCTTCGCAGTCAATCCTGGACTTCTTGAAACGCCACCG |
| | Pol:367-431 | GTGGTCGCTAAAGAGATCGTTGCGAGCTGTGATAAATGTCAACTTAAAGGCGAGGCTATGCATGGCCAGGTC |
| | Pol:932-1003 | GACTGTAGCCCGGGCATCTGGCAGCTGGATTGCACTCACCTGGAGGGTAAGATCATTCTCGTGGCGGTCCAT |
| | Pol:129-320 | GTTGCCAGTGGCTACATTGAGGCGGAGGTGATTCCTGCGGAAACTGGTCAGGAGACAGCCTATTTCTTGCTG |
| | | AAGCTCGCGGGACGCTGGCCTGTCAAAACTAAGGAAAAGGTTTATTTGGCCTGGGTTCCCGCACATAAAGGA |
| | | ATTGGTGGCAATGAACAGGTAGACAAACTTGTAAGTACTCAGGGATATTTTCCCGATTGGCAGAATTACACT |
| | | CCAGGGCCGGGGACTAGGTACCCTTTGACATTTGGTTGGTGTTTTAAGCTTGTGCCTGTTCGGGCGAAGAGG |
| | | GCGCCAGTCAAACAGACTCTGAATTTCGACCTGCTGAAGCTGGCAGGAGACGTCGAGTCCAACCCTGGTCCT |
| | | ATGTTCCCACAGATTACTCTGTGGCAGCGCCCGCTTGTGACTATTAAAATCGGCGGACAACTCAAAGAGGCA |
| | | CTCCTTGACACCGGAGCGGACGACACGGTGCTGGAAGAAATGAACTTGCCCGGCCGGTGGAAGCCAAAGATG |
| | | ATCGGAGGTATCGGCGGCTTTATAAAGGTGCGCCAGTATGACCAAGAAGAAGTCGGCTTCCCAGTAAAGCCT |
| | | CAAGTTCCACTGAGACCTATGACTTTTAAGGGTGCGCTTGATCTGTCACACTTCCTCCGAGAGAAAGGCGGC |
| | | TTGGAGGGCCTTATTCCCAAGTTCAAGTTGCCTATTCAAAAAGAAACGTGGGAGACGTGGTGGACTGAATAT |
| | | TGGCAGGCGACCTGGATCCCTGAATGGGAGTTCGTGAACACACCCCCACTCGTTAAACTCTGGTATCAGTTG |
| | | GAAAAGGAACCCATCGTGGGCGCCGAGACATTTTACGTCGATGGTGCCGCTAACAGAGAGACCAAGTGGGGG |
| | | TTTACAACGCCTGACAAGAAGCACCAGAAGGAGCCCCCTTTCCTTTGGATGGGATATGAGTTGCACCCCGAC |
| | | AAATGGACCGTGCAACCGATTGTCTTGCCTGAGAAAGACTCTTGGACAGTGAACGATATCCAAAAACTTGTG |
| | | GGAAAATTGAATTGGGCAAGCCAAATCTACCCAGGGATAAAGGTAATCACTAAGATTCAAAACTTCCGAGTA |
| | | TACTACCGAGACAGCAGAGATCCCTTGTGGAAAGGTCCTGCGAAACTGCTCTGGAAAGGCGAGGGAGCTGTG |
| | | GTCATTCAGGACAACTCAGACATCAAAGTAGTCCCACGCCGCAAAGCGAAAATCATACGCGACTATGGCAAA |
| | | CAAATGGCAGGTGATGATTGTGTGGCGAGTCGACAAGATGAGGATGGTACCGTTCTGGTCGGGCCGACACCT |
| | | GTTAATATTATAGGACGCAATTTGTTGACACAAATCGGCTGCACTCTTAACTTCCCGATAAGTCCCATCGAG |
| | | ACAGTGCCAGTTAAATTGAAGCCAGGGATGGACGGTCCTAAGGTTAAGCAGTGGCCCCTCACTGAAGAAAAA |
| | | ATCAAGGCTCTCGTGGAAATTTGCACTGAGATGGAGAAGGAGGGCAAAATCTCCAAGATAGGTCCAGAGAAC |
| | | CCATATAATACGCCGGTATTTGCAATCAAAAAAAAGGACAGCACAAAGTGGCGAAAGCTGGTTGACTTTCGA |
| | | GAGCTGAACAAGCGGACGCAGGACTTTTGGGAAGTCCAATTGGGAATACCGCATCCCGCTGGATTGAAAAAA |
| | | AAAAAGAGCGTAACAGTCCTCGATGTAGGTGATGCATACTTCAGTGTCCCACTCGATAAAGATTTTAGAAAG |
| | | TACACTGCTTTTACGATTCCATCCATTAACAACGAGACTCCCGGTATTCGATATCAATACAATGTACTCCCA |
| | | CAAGGTTGGAAAGGCTCACCTGCGATCTTCCAAAGTAGCATGACT |
| 219 | M | ATGTTGTCTCCTAGAACTCTGAATGCTTGGGTAAAGGTGATCGAAGAAAAGGCATTCTCACCCGAGGTTATC |
| | Gag:147-369 | CCTATGTTTACTGCGTTGAGCGAAGGCGCAACACCCCATGATCTGAACACAATGCTGAATACAATCGGCGGA |
| | Pol:747-827 | CATCAAGCTGCTATGCAAATGCTTAAGGACACCATCAATGAGGAGGCAGCCGAGTGGGATCGCGTTCATCCA |
| | Gag:1-53 | GTCCACGCTGGGCCCGTTGCGCCTGGTCAGATGAGGGACCCACGAGGATCCGACATCGCAGGGAGCACCAGT |
| | AA | ACACTCCAAGAGCAGATTGCATGGATGACGAACAATCCACCAATACCTGTCGGTGACATTTACAAAAGGTGG |
| | Pol:840-920 | ATTATTATGGGCTTGAACAAAATTGTACGGATGTATAGCCCGGTGAGCATACTGGACATTAAACAGGGTCCA |
| | Nef:117-148 | AAAGAACCCTTTCGAGATTACGTTGACAGATTTTATAGGACACTGAGGGCGGAACAAGCGTCTCAAGACGTT |
| | LIK | AAAAACTGGATGACAGAAACGCTGCTTGTACAGAATTCCAACCCAGACTGCAAAACTATACTGAAAGCGCTC |
| | Pol:683-708 | GGTCCCGGTGCGACACTTGAGGAAATGATGAGCGCATGCCAGGGCGTCGGAGGGCCGTCTCACAAGGCCCGC |
| | RAKR | GTGTTGGCGGAAGCTATGTGCCAGGTGGCTAAGGAGATAGTAGCATGTTGTGATAAATGTCAGCTTAAAGGT |
| | F2A linker | GAAGCTATACATGGGCAGGTGGATTGTAGCCCGGGTGTATGGCAGCTGGACTGTACTCATCTGGAAGGAAAG |
| | M | GTAATACTTGTCGCAGTTCATGTCGCGAGCGGATACATTGAAGCTGAAATCATTCCTACGGAGACGGGCCAA |
| | Pol:56-117 | GAGACAGCTTACTTCATACTTAAACTTGCTGGGCGATGGCCGGTGACAACTATGGCGGCGCGCGCTTCAATT |
| | Pol:367-431 | TTGAGTGGTGGAAAGTTGGACAAGTGGGAGAAGATTAGACTCAGACCCGGAGGGAGAAAGAAGTATAAACTG |
| | AA | AAACATCTGGTTTGGGCTTCACGCGAACTTGAACGGTTTGCTCTCAACCCCGGGCTGCTCGAAACTGCTGCT |
| | QEE | GCTGTAAAGGCCGCTTGTTGGTGGGCGGGGGTAAAGCAGGAATTCGGAATTCCATATAACACTCAAAGCCAG |
| | Nef:64-99 | GGAGTAGTGGAATCCATGAATAATGAACTTAAGAAGATAATTGGACAGATTCGCGATCAGGCTGAACATCTC |
| | Pol:932-1003 | AAGACGGCCGTACAAATGGCAGTATTGATTCATAACTTTAAACGGAAGGGCGGCATAGGAGAGTATTCTGCG |
| | Pol:542-606 | GGAGAACGCATAATAGATATAATTGCGACTCAGGGGTTCTTTCCGGATTGGCAGAACTATACGCCGGGGCCA |
| | AA | GGCATTAGGTTCCCCCTCACGTTTGGATGGTGTTTCAAGTTGGTACCGTTGCTCATTAAAAAAGAAAAAATC |
| | Pol:129-320 | TACCTGGCCTGGGTCCCGGCGCACAAGGGTATAGGGGGGAACGAGCAAATTGATAAGCTCGTGTCAAGGGCG |
| | | AAGCGCGCGCCAGTCAAACAGACCTTGAATTTCGACCTCCTTAAGCTCGCTGGAGACGTCGAATCCAACCCT |
| | | GGCCCGATGCTGCCACAAATCACATTGTGGCAACGACCCATTGTAACAATAAAAATCGGGGGCCAGATCAAA |
| | | GAAGCGCTGCTTGACACCGGCGCCGACGATACAGTCCTCGAGGATATGAATTTGCCAGGCAAATGGAAGCCG |
| | | AAGATGATTGGCGGCATTGGCGGCTTTATTAAGGTTAAACAGTATGATCAGTGGGGATTGACCACTCCCGAT |
| | | AAGAAGCATCAGAAAGATCCGCCTTTTCTGTGGATGGGGTACGAACTGCACCCTGATCGATGGACGGTCCAG |
| | | CCGATAGAGCTGCCGGAAAAAGAATCATGGACCGTGAATGATATTCAAAAACTGATCGGAAAACTCAATTGG |
| | | GCGTCCCAGATATATGCTGGCATCAAAGTTGCAGCACAAGAAGAGGAAGAGGTAGGTTTCCCGGTTCGGCCG |
| | | CAAGTTCCCTTGCGACCGATGACATACAAGGGCGCATTGGACCTTTCTCACTTCCTCAAGGAAAAGGGCGGT |
| | | TTGGAGGGCATCACTAAACTTCAGAATTTCAGAGTCTACTATAGAGATAACAGGGACCCATTGTGGAAGGGC |
| | | CCCGCTCGACTTCTCTGGAAAGGGGAGGGAGCGGTTGTAATTCAAGACAACAGTGAAATTAAGGTCGTCCCA |
| | | CGACGGAAGGTTAAAATAATTCGCGACTATGGCAAGCGAATGGCGGGAGACGACTGTGTAGCAGGACGACAA |
| | | GACGAGGACCCAAAGTTTAGATTGCCGATCCAGAAAGAGACATGGGATACGTGGTGGACGGACTATTGGCAG |
| | | GCCACCTGGATACCAGAATGGGAGTTTACAAACACTCCTCCACTCGTGAAATTGTGGTATCAACTTGAGACC |
| | | GAGCCCATAGCTGGTGTAGAGACGTTTTACGTTGACGGTGCTAGCAACAGGGAAACAAAGGCCGCTGGAACC |
| | | GTGCTCATCGGTCCTACTCCTGTGAACATAATTGGACGAAATTTGTTGACCCAGCTGGGATGCACCCTCAAT |
| | | TTCCCCATTAGCCCAATAGATACCGTACCAGTCAAGCTTAAGCCTGGTATGGACGGTCCGCGAGTTAAGCAA |
| | | TGGCCACTTACTGAGGAGAAAATCAAGGCACTCATCGAGATCTGCACCGAAATGGAGAAGGAGGGCAAAATA |
| | | AGCAGGATTGGTCCCGAGAATCCATATAATACGCCGATCTTCGCGATAAAAAAGAAGGACGGCACCAAATGG |
| | | CGAAAACTGGTTGACTTCCGGGAGCTTAACAAAAAAACTCAGGATTTTTGGGAAGTTCAACTCGGGATCCCA |
| | | CACCCGTCTGGTCTTAAAAAAAAAAAAAGCGTAACAGTCCTTGACATCGGCGATGCCTACTTTAGCGTGCCT |
| | | CTCGATAAGGAGTTCAGAAAATACACGGCTTTCACTGTACCAAGCACAAACAATGAGACTCCTGGGGTCAGA |
| | | TACCAGTACAATGTCCTTCCCATGGGGTGGAAAGGAAGCCCCGCAATATTCCAGTGCTCAATGACG |
| 226 | M | ATGCTTTCACCGAGAACCTTGAACGCGTGGGTTAAAGTAATCGAAGAGAAGGCATTTAGCCCGGAGGTAATC |
| | Gag:147-369 | CCCATGTTTACCGCACTTTCCGAAGGTGCTACACCCCACGACCTGAATACAATGTTGAATACTATTGGCGGC |
| | Pol:747-827 | CACCAAGCTGCCATGCAGATGCTTAAGGACACTATTAACGAGGAAGCCGCCGAATGGGATCGCGTTCACCCT |
| | Gag:1-53 | GTTCACGCGGGTCCAGTCGCACCAGGACAAATGCGAGACCCTCGGGGGTCTGACATCGCAGGATCAACTAGT |
| | AA | ACATTGCAGGAACAGATTGCTTGGATGACCAATAACCCTCCGATCCCGGTAGGTGACATTTATAAACGGTGG |
| | Pol:840-920 | ATCATCATGGGATTGAACAAAATAGTCCGAATGTACAGTCCAGTGAGTATTCTTGACATTAAACAGGGACCA |
| | Nef:117-148 | AAGGAGCCGTTCCGGGACTACGTTGACCGGTTTTACAGGACCCTGAGAGCCGAACAGGCTTCCCAGGATGTG |
| | LIK | AAAAACTGGATGACAGAGACCCTGCTTGTTCAGAATTCAAATCCTGACTGTAAAACGATTCTGAAGGCACTC |
| | Pol:683-708 | GGTCCCGGCGCCACGCTGGAGGAAATGATGTCAGCTTGTCAGGGAGTAGGAGGACCTTCTCATAAGGCACGC |
| | F2A | GTCCTTGCAGAGGCCATGTGCCAAGTCGCGAAAGAGATCGTGGCCTGTTGTGACAAGTGCCAACTTAAGGGA |
| | M | GAAGCCATCCATGGGCAGGTCGATTGTTCTCCGGGCGTTTGGCAATTGGACTGCACGCACTTGGAGGGTAAG |
| | Pol:56-117 | GTGATTCTTGTTGCCGTTCATGTCGCAAGCGGTTACATTGAAGCGGAGATTATCCCAACTGAGACCGGCCAA |
| | Pol:367-431 | GAGACTGCATATTTCATCCTTAAACTGGCGGGAAGATGGCCGGTCACGACTATGGCTGCACGAGCATCCATA |
| | AA | CTGTCTGGGGGAAAGCTCGACAAGTGGGAGAAGATTCGACTTAGGCCTGGAGGAAGAAAAAAGTATAAGTTG |
| | QEE | AAACATCTTGTTTGGGCATCACGAGAACTTGAACGGTTTGCTCTGAATCCGGGATTGCTTGAAACCGCCGCG |
| | Nef:64-99 | GCTGTAAAGGCAGCATGTTGGTGGGCTGGTGTCAAACAAGAGTTCGGTATTCCCTATAACACCCAAAGTCAA |
| | Pol:932-1003 | GGCGTCGTAGAATCCATGAACAATGAACTTAAGAAAATTATCGGACAAATCCGGGACCAAGCGGAACACCTC |
| | Pol:542-60 | AAAACTGCTGTCCAGATGGCAGTTCTGATTCATAACTTTAAAAGAAAGGGGGGGATAGGGGAGTATTCTGCA |
| | AA | GGTGAACGAATTATAGACATAATTGCCACACAAGGGTTTTTTCCAGATTGGCAAAATTATACACCGGGCCCT |
| | Pol:129-320 | GGGATAAGATTCCCGCTCACCTTCGGCTGGTGCTTCAAACTGGTACCCTTGCTGATCAAGAAAGAGAAGATC |
| | | TATCTTGCTTGGGTGCCAGCCCATAAAGGGATCGGGGGTAACGAACAAATCGATAAGCTGGTGTCTAGAGCT |
| | | AAACGGGCTCCCGTAAAACAGACCTTGAACTTCGATCTGCTTAAATTGGCAGGGGACGTGGAAAGCAACCCC |
| | | GGGCCAATGCTGCCCCAGATAACACTTTGGCAACGCCCCATCGTGACAATCAAGATCGGTGGCCAAATTAAG |
| | | GAAGCACTCTTGGACACGGGAGCAGACGACACTGTGCTGGAGGATATGAACCTGCCGGGCAAGTGGAAACCA |
| | | AAGATGATCGGGGGCATTGGCGGGTTCATAAAGGTTAAACAGTACGACCAATGGGGGTTGACAACGCCTGAT |
| | | AAGAAGCATCAAAAAGATCCCCCATTTTTGTGGATGGGTTATGAACTTCACCCGGACAGGTGGACCGTTCAG |
| | | CCGATAGAGCTCCCAGAAAAGGAGTCTTGGACAGTTAATGACATACAGAAACTTATTGGCAAACTTAACTGG |
| | | GCTTCACAGATTTATGCCGGCATCAAAGTCGCCGCCCAGGAAGAAGAAGAGGTAGGTTTTCCCGTACGACCT |
| | | CAGGTTCCTCTTCGGCCTATGACCTATAAGGGTGCGCTTGATCTTTCTCACTTCCTTAAAGAAAAAGGAGGT |
| | | CTGGAAGGTATCACGAAACTTCAGAATTTTCGGGTGTATTACCGGGACAACAGAGACCCGCTTTGGAAGGGG |
| | | CCGGCTAGGCTTCTGTGGAAAGGCGAGGGAGCGGTAGTTATCCAGGATAACTCTGAGATAAAGGTAGTACCC |
| | | CGACGGAAGGTAAAGATCATCAGAGACTACGGCAAGAGGATGGCTGGAGACGACTGTGTGGCCGGGCGACAG |
| | | GATGAAGATCCTAAATTCAGGCTGCCAATCCAAAAAGAGACGTGGGACACATGGTGGACCGATTATTGGCAG |
| | | GCTACGTGGATCCCCGAATGGGAGTTTACCAATACTCCGCCACTCGTGAAGCTTTGGTACCAATTGGAGACA |
| | | GAGCCTATAGCCGGCGTTGAGACCTTCTACGTGGATGGGGCCAGCAACAGAGAAACCAAAGCGGCCGGAACG |
| | | GTCCTGATCGGTCCCACACCTGTTAACATCATAGGGCGCAATCTGCTTACGCAATTGGGGTGCACATTGAAT |
| | | TTTCCAATATCCCCTATTGATACCGTGCCGGTTAAATTGAAGCCGGGTATGGACGGGCCTCGGGTCAAGCAG |
| | | TGGCCCCTGACCGAAGAAAAGATCAAAGCCCTGATTGAGATCTGCACGGAAATGGAAAAAGAGGGCAAGATT |
| | | AGTCGCATCGGCCCGGAGAACCCATACAATACTCCTATTTTTGCAATTAAAAAAAAGGACGGAACAAAGTGG |
| | | AGGAAACTTGTAGATTTCAGAGAGCTTAATAAGAAAACTCAGGACTTCTGGGAGGTCCAACTCGGTATTCCG |
| | | CATCCCTCCGGACTTAAGAAGAAAAAGTCAGTAACCGTCTTGGATATAGGGGACGCTTATTTTTCAGTGCCC |
| | | CTCGATAAAGAATTTCGCAAATACACGGCGTTTACTGTGCCATCTACTAATAACGAAACGCCAGGCGTGAGA |
| | | TATCAATACAACGTCCTTCCTATGGGCTGGAAGGGTTCACCCGCAATTTTTCAGTGCTCCATGACC |
| 225 | Gag:147-369 | ATTTCTCCTCGGACGCTGAATGCATGGGTGAAGGTAGTGGAGGAAAAGGCATTTTCACCAGAAGTCATTCCG |
| | Pol:747-827 | ATGTTCTCCGCGCTCTCCGAGGGTGCTACGCCACAGGACTTGAATACGATGCTGAATACCGTTGGTGGCCAT |
| | Gag:1-53 | CAGGCGGCGATGCAGATGTTGAAGGAGACAATTAACGAAGAAGCCGCCGAATGGGACAGATTGCACCCGGTG |
| | AA | CATGCGGGGCCAATAGCTCCTGGCCAGATGCGCGAGCCTAGGGGTTCTGACATTGCTGGTACAACAAGTACC |
| | Pol:840-920 | CTTCAGGAGCAGATTGGTTGGATGACGAATAACCCTCCCATACCTGTTGGCGAGATCTATAAGCGCTGGATT |
| | Nef:117-148 | ATACTTGGGCTGAATAAGATAGTCCGAATGTATTCTCCCACCTCTATTCTGGATATTCGGCAAGGACCTAAG |
| | LIK | GAGCCGTTTAGAGACTACGTAGACCGGTTTTACAAAACCCTGCGGGCGGAACAAGCTTCTCAGGAAGTCAAA |
| | Pol:683-708 | AATTGGATGACTGAGACCTTGCTCGTCCAGAATGCGAACCCGGACTGTAAAACAATACTCAAAGCGCTGGGC |
| | F2A | CCCGCTGCAACCCTGGAAGAAATGATGACGGCTTGTCAGGGAGTAGGAGGCCCCGGACATAAGGCACGAGTG |
| | Pol:56-117 | TTGGCAGAAGCCATGAGCCAGCCGCCTGTCGTCGCGAAAGAAATCGTCGCTTCTTGCGACAAATGTCAGCTG |
| | Pol:367-431 | AAGGGGGAGGCGATGCACGGTCAAGTTGATTGCTCTCCCGGTATTTGGCAATTGGACTGTACCCACCTTGAA |
| | AA | GGCAAAATTATTCTGGTTGCAGTGCACGTAGCATCCGGTTACATCGAAGCTGAAGTGATACCCGCAGAGACA |
| | QEE | GGCCAGGAGACGGCTTATTTCCTCCTTAAGCTTGCGGGTCGGTGGCCCGTAAAGACCATGGCTGCTCGGGCA |
| | Nef:64-99 | TCTGTCCTCTCCGGTGGTGAACTCGACCGATGGGAAAAGATTCGATTGCGCCCCGGAGGAAAGAAGAAATAT |
| | Pol:932-1003 | AGGCTGAAACATATTGTGTGGGCATCACGGGAACTTGAGCGATTTGCGGTAAACCCAGGCCTTTTGGAAACA |
| | Pol:542-606 | GCTGCAACTGTGAAAGCGGCTTGCTGGTGGGCGGGGATAAAACAGGAGTTTGGTATCCCGTACAATCCCCAA |
| | AA | TCTCAGGGGGTAGTAGAAAGCATGAACAAAGAATTGAAAAAAATAATTGGCCAGGTTCGCGACCAAGCCGAG |
| | Pol:129-320 | CACCTCAAGACCGCTGTACAGATGGCTGTATTTATTCACAACTTCAAGCGGAAGGGCGGAATAGGAGGATAT |
| | | AGCGCAGGGGAAAGGATTGTTGATATTATTGCAACACAAGGTTACTTTCCTGACTGGCAAAACTACACACCG |
| | | GGCCCTGGCACGCGCTATCCCCTTACGTTCGGTTGGTGCTTCAAGCTGGTGCCGGTAAAAGAAAAAGTTTAT |
| | | TTGGCATGGGTTCCTGCACATAAAGGAATAGGGGGTAACGAACAAGTTGACAAACTCGTCAGCCGCGCTAAA |
| | | AGAGCCCCAGTCAAGCAGACCCTGAATTTTGACCTGCTTAAATTGGCTGGGGACGTCGAGAGTAACCCGGGA |
| | | CCCTTCCCACAAATTACACTCTGGCAGCGACCACTGGTAACAATCAAAATAGGGGGACAATTGAAAGAAGCA |
| | | CTCCTGGATACGGGCGCGGACGATACAGTCCTGGAGGAAATGAATCTCCCCGGCCGCTGGAAACCTAAGATG |
| | | ATAGGGGGGATCGGTGGATTTATTAAAGTGCGGCAGTACGATCAATGGGGTTTTACGACACCTGACAAGAAA |
| | | CATCAAAAGGAGCCGCCATTTCTTTGGATGGGTTATGAGCTTCATCCGGATAAATGGACTGTTCAGCCGATT |
| | | GTCCTCCCCGAGAAGGATAGTTGGACTGTGAACGACATCCAGAAGCTGGTCGGGAAACTTAATTGGGCCAGT |
| | | CAAATATATCCAGGTATTAAAGTTGCCGCACAAGAAGAGGAGGAAGTAGGGTTCCCCGTGAAACCGCAAGTC |
| | | CCTCTCCGGCCCATGACCTTTAAGGGCGCTCTCGACCTGTCCCATTTCCTCCGCGAAAAGGGGGGTTTGGAA |
| | | GGCTTGATCATTACCAAGATTCAAAACTTCAGGGTCTATTATCGAGACAGTCGCGATCCCCTTTGGAAAGGA |
| | | CCTGCGAAACTTCTTTGGAAAGGAGAAGGAGCCGTGGTAATTCAGGATAATTCTGACATAAAGGTCGTCCCA |
| | | CGCCGAAAAGCGAAGATTATAAGAGATTATGGCAAGCAGATGGCCGGGGATGATTGTGTCGCAAGTAGACAG |
| | | GATGAAGACCCTAAATTCAAGCTTCCAATCCAGAAGGAAACGTGGGAGACATGGTGGACCGAGTATTGGCAA |
| | | GCTACTTGGATCCCAGAATGGGAATTTGTGAACACTCCCCCACTCGTAAAGCTGTGGTACCAGCTTGAAAAA |
| | | GAACCTATAGTCGGGGCGGAGACGTTCTATGTAGACGGCGCCGCTAATCGAGAGACAAAAGCAGCAGGCACG |
| | | GTACTGGTAGGCCCGACCCCTGTCAACATCATTGGACGAAATCTGTTGACCCAGATTGGGTGTACCCTTAAC |
| | | TTTCCCATTTCACCAATAGAGACCGTCCCGGTTAAACTGAAACCGGGTATGGATGGTCCCAAAGTAAAACAG |
| | | TGGCCACTTACCGAGGAGAAGATTAAGGCACTCGTTGAAATATGTACAGAAATGGAAAAAGAGGGGAAAATC |
| | | TCTAAAATTGGCCCTGAAAATCCGTACAACACTCCGGTATTCGCCATAAAAAAGAAGGACTCTACCAAGTGG |
| | | CGCAAACTCGTGGACTTTAGAGAACTGAATAAAAGGACCCAGGACTTTTGGGAAGTCCAGCTGGGTATTCCT |
| | | CACCCCGCTGGTCTCAAGAAAAAGAAAAGTGTCACTGTCTTGGATGTTGGAGATGCGTACTTTTCAGTACCT |
| | | CTTGATAAAGATTTTCGAAAGTATACCGCGTTTACCATTCCCTCCATAAATAACGAAACACCGGGGATCAGG |
| | | TATCAATATAACGTGCTTCCACAAGGCTGGAAGGGTTCACCGGCTATTTTCCAATCTTCTATGACG |

As appropriate, in certain embodiments, 3′-end of the polynucleotide encoding the fusion polypeptides or compound fusion polypeptides described herein comprises one or multiple tandem stop codons, e.g., two or more tandem TAG (“amber”), TAA (“ochre”) or TGA (“opal” or “umber”) stop codons. The multiple tandem stop codons can be the same or different.

As appropriate, in certain embodiments, 3′-end of the polynucleotide encoding the fusion polypeptides or compound fusion polypeptides described herein does not comprise a poly A sequence. As appropriate, in certain embodiments, 3′-end of the polynucleotide encoding the fusion polypeptides or compound fusion polypeptides described herein comprises a poly A sequence.

Further provided are expression cassettes, comprising a polynucleotide encoding a fusion polypeptide or a compound fusion polypeptide, as described herein, operably linked to one or more regulatory sequences, e.g., a promoter. In some embodiments, the polynucleotide is operably linked to and under the control of a constitutive promoter or an inducible promoter. In some embodiments, the promoter is selected from cytomegalovirus major immediate-early (CMV), the CMV enhancer fused to the chicken beta-actin promoter (CAG), human elongation factor-la (HEF-la), mouse cytomegalovirus (mouse CMV), Chinese hamster elongation factor-la (CHEF-la), and phosphoglycerate kinase (PGK). In some embodiments, the promoter is a native promoter of the viral expression vector, e.g., an arenavirus vector promoter, an adenovirus vector promoter, etc.

Further provided are methods for making a fusion polypeptide or compound fusion polypeptide, pharmaceutical composition, immunogenic composition or vaccine composition comprising same. In some implementations, the methods comprise constructing the fusion polypeptides or compound fusion polypeptides using peptide synthesis. In some implementations, the methods comprise constructing, using synthetic or recombinant DNA technology, polynucleotides encoding each of the polypeptides of the bivalent antigen and expressing the polypeptides from an expression vector. In some implementations, the methods may further comprise inserting the polynucleotides into one or more vectors and expressing the encoded polypeptides in a cell.

### 4. Vectors and Host Cells

Further provided are vectors comprising one or more polynucleotides encoding one or more of the fusion polypeptides or compound fusion polypeptides, described herein, or an expression cassette comprising such polynucleotides. A vector can be of any type, for example, a recombinant vector such as an expression vector. Vectors include without limitation, plasmids, cosmids, bacterial artificial chromosomes (BAC) and yeast artificial chromosomes (YAC) and vectors derived from bacteriophages or plant or animal (including human) viruses. Vectors can comprise an origin of replication recognized by the proposed host cell and in the case of expression vectors, promoter and other regulatory regions recognized by the host cell. In additional embodiments, a vector comprises one or more polynucleotides encoding one or more fusion polypeptides or one or more compound fusion polypeptides, as described herein, operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include without limitation, those suitable for recombinant production of the fusion polypeptides and/or compound fusion polypeptides, disclosed herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The choice of the vector is dependent on the recombinant procedures followed and the host used. Introduction of vectors into host cells can be effected by inter alia calcium phosphate transfection, DEAE-dextran-mediated transfection, lipofectamine transfection, electroporation, virus infection, or via administration to a subject, as described herein. Vectors may be autonomously replicating or may replicate together with the chromosome into which they have been integrated. In certain embodiments, the vectors contain one or more selection markers. The choice of the markers may depend on the host cells of choice. These include without limitation, kanamycin, neomycin, puromycin, hygromycin, zeocin, thymidine kinase gene from Herpes simplex virus (HSV-TK), and dihydrofolate reductase gene from mouse (dhfr). Vectors comprising one or more nucleic acid molecules encoding the fusion polypeptides or compound fusion polypeptides, described herein, operably linked to one or more nucleic acid molecules encoding proteins or peptides that can be used to isolate the fusion polypeptides or compound fusion polypeptides, are also contemplated. These proteins or peptides include without limitation, glutathione-S-transferase, maltose binding protein, metal-binding polyhistidine, green fluorescent protein, luciferase and beta-galactosidase.

In various embodiments, the vector comprises one or more polynucleotides encoding one or more fusion polypeptides comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In various embodiments, the vector comprises one or more polynucleotides encoding one or more fusion polypeptides comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223.

In various embodiments, the vector comprises a polynucleotide encoding a fusion polypeptide comprising or consisting of an amino acid sequence of SEQ ID NO: 82, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:82. In various embodiments, the vector comprises a polynucleotide encoding a fusion polypeptide comprising or consisting of an amino acid sequence of SEQ ID NO: 83, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:83. In various embodiments, the vector comprises a polynucleotide encoding a fusion polypeptide comprising or consisting of an amino acid sequence of SEQ ID NO: 85, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:85. In various embodiments, the vector comprises a polynucleotide encoding a fusion polypeptide comprising or consisting of an amino acid sequence of SEQ ID NO: 86, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:86. In various embodiments, the vector comprises a polynucleotide encoding a fusion polypeptide comprising or consisting of an amino acid sequence of SEQ ID NO: 87, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 87. In various embodiments, the vector comprising a polynucleotide encoding one or more of the foregoing fusion polypeptides is a Cali mammarenavirus (a.k.a., Pichinde mammarenavirus or Pichinde arenavirus.

In various embodiments, the vector comprises a polynucleotide encoding a fusion polypeptide comprising or consisting of an amino acid sequence of SEQ ID NO: 85, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:85. In various embodiments, the vector comprises a polynucleotide encoding a fusion polypeptide comprising or consisting of an amino acid sequence of SEQ ID NO: 98, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:98. In various embodiments, the vector comprises a polynucleotide encoding a fusion polypeptide comprising or consisting of an amino acid sequence of SEQ ID NO: 99, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:99. In various embodiments, the vector comprises a polynucleotide encoding a fusion polypeptide comprising or consisting of an amino acid sequence of SEQ ID NO: 100, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 100. In various embodiments, the vector comprises a polynucleotide encoding a fusion polypeptide comprising or consisting of an amino acid sequence of SEQ ID NO: 101, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 101. In various embodiments, the vector comprising a polynucleotide encoding one or more of the foregoing fusion polypeptides is a Lymphocytic choriomeningitis mammarenavirus (LCMV).

In various embodiments, the vector comprises a polynucleotide encoding a fusion polypeptide comprising or consisting of an amino acid sequence of SEQ ID NO: 209, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209. In various embodiments, the vector comprises a polynucleotide encoding a fusion polypeptide comprising or consisting of an amino acid sequence of SEQ ID NO: 222, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222. In various embodiments, the vector comprises a polynucleotide encoding a fusion polypeptide comprising or consisting of an amino acid sequence of SEQ ID NO: 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In various embodiments, the vector comprises a polynucleotide encoding a fusion polypeptide comprising or consisting of an amino acid sequence of SEQ ID NO: 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 227.

In various embodiments, the vector comprises one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In various embodiments, the vector comprises one or more polynucleotides encoding the following first fusion polypeptide and second fusion polypeptide, optionally joined or connected by one or more linkers: SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively.

In various embodiments, the vector comprises one or more polynucleotides encoding the following first fusion polypeptide and second fusion polypeptide, optionally joined or connected by one or more linkers: SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively.

In various embodiments, the vector comprises one or more polynucleotides encoding the following first fusion polypeptide and second fusion polypeptide, optionally joined or connected by one or more linkers: SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively.

In various embodiments, the vector comprises one or more polynucleotides encoding the following first fusion polypeptide and second fusion polypeptide, optionally joined or connected by one or more linkers: SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively.

In various embodiments, the vector comprises one or more polynucleotides encoding the following first fusion polypeptide and second fusion polypeptide, optionally joined or connected by one or more linkers: SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively.

In various embodiments, the vector comprises one or more polynucleotides encoding the following first fusion polypeptide and second fusion polypeptide, optionally joined or connected by one or more linkers: SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively.

In various embodiments, the vector comprises one or more polynucleotides encoding the following first fusion polypeptide and second fusion polypeptide, optionally joined or connected by one or more linkers: SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively.

In various embodiments, the vector comprises one or more polynucleotides encoding the following first fusion polypeptide and second fusion polypeptide, optionally joined or connected by one or more linkers: SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively.

In various embodiments, the vector comprises one or more polynucleotides encoding the following first fusion polypeptide and second fusion polypeptide, optionally joined or connected by one or more linkers: SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In various embodiments, the vector comprises one or more polynucleotides encoding the following first fusion polypeptide and second fusion polypeptide, optionally joined or connected by one or more linkers: SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively.

In various embodiments, the vector comprises one or more polynucleotides encoding the following first fusion polypeptide and second fusion polypeptide, optionally joined or connected by one or more linkers: SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively.

In various embodiments, the vector comprises one or more polynucleotides encoding the following first fusion polypeptide and second fusion polypeptide, optionally joined or connected by one or more linkers: SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively.

In various embodiments, the vector comprises one or more polynucleotides encoding the following first fusion polypeptide and second fusion polypeptide, optionally joined or connected by one or more linkers: SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively.

In various embodiments, the vector comprises one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively.

In various embodiments, the vector comprises one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively.

In various embodiments, the vector comprises one or more polynucleotides encoding the following first fusion polypeptide and second fusion polypeptide, optionally joined or connected by one or more linkers: SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively.

In various embodiments, the vector comprises one or more polynucleotides encoding the following first fusion polypeptide and second fusion polypeptide, optionally joined or connected by one or more linkers: SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively.

In various embodiments, the vector comprises one or more polynucleotides encoding the following first fusion polypeptide and second fusion polypeptide, optionally joined or connected by one or more linkers: SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In various embodiments, the vector comprises one or more polynucleotides encoding the following first fusion polypeptide and second fusion polypeptide, optionally joined or connected by one or more linkers: SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively.

In various embodiments, the vector comprises one or more polynucleotides encoding the following first fusion polypeptide and second fusion polypeptide, optionally joined or connected by one or more linkers: SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In various embodiments, the vector comprises one or more polynucleotides encoding the following first fusion polypeptide and second fusion polypeptide, optionally joined or connected by one or more linkers: SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively.

In various embodiments, the vector comprises one or more polynucleotides encoding the following first fusion polypeptide and second fusion polypeptide, optionally joined or connected by one or more linkers: SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively.

In various embodiments, the vector comprises one or more polynucleotides encoding the following first fusion polypeptide and second fusion polypeptide, optionally joined or connected by one or more linkers: SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In various embodiments, the vector comprises one or more polynucleotides encoding the following first fusion polypeptide and second fusion polypeptide, optionally joined or connected by one or more linkers: SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In various embodiments, the vector comprises a polynucleotide encoding a compound fusion polypeptide comprising SEQ ID NO: 105 or 206, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105 or 206.

In various embodiments, the vector comprises a polynucleotide encoding a compound fusion polypeptide comprising SEQ ID NO: 107 or 207, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107 or 207.

In various embodiments, the vector comprises a polynucleotide encoding a compound fusion polypeptide comprising SEQ ID NO: 109, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 109.

In various embodiments, the vector comprises a polynucleotide encoding a compound fusion polypeptide comprising SEQ ID NO: 111, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 111.

In various embodiments, the vector comprises a polynucleotide encoding a compound fusion polypeptide comprising SEQ ID NO: 200, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200.

In various embodiments, the vector comprises a polynucleotide encoding a compound fusion polypeptide comprising SEQ ID NO: 201, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201.

In various embodiments, the vector comprises a polynucleotide encoding a compound fusion polypeptide comprising SEQ ID NO: 202, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202.

In various embodiments, the vector comprises a polynucleotide encoding a compound fusion polypeptide comprising SEQ ID NO: 203, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203.

In various embodiments, the vector comprises a polynucleotide encoding a compound fusion polypeptide comprising SEQ ID NO: 204, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204.

In various embodiments, the vector comprises a polynucleotide encoding a compound fusion polypeptide comprising SEQ ID NO: 205, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205.

In various embodiments, the vector comprises a polynucleotide encoding a compound fusion polypeptide comprising SEQ ID NO: 208, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208.

In various embodiments, the vector comprises a polynucleotide encoding a compound fusion polypeptide comprising SEQ ID NO: 209, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209.

In various embodiments, the vector comprises a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222.

In various embodiments, the vector comprises a polynucleotide encoding a compound fusion polypeptide comprising SEQ ID NO: 223, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223.

In various embodiments, the vector comprises a polynucleotide encoding a compound fusion polypeptide comprising SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 227.

In some embodiments, the vector comprises a polynucleotide encoding a fusion polypeptide or a compound fusion polypeptide, as described herein, which has a nucleic acid sequence of any one of SEQ ID NOs: 130-167, 210-219 and 225-226, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from SEQ ID NOs: 130-167, 210-219 and 225-226. In some embodiments, the vector comprises a polynucleotide encoding a fusion polypeptide or a compound fusion polypeptide, as described herein, which has a nucleic acid sequence of any one of SEQ ID NOs: 139, 140, 142, 143, 145, 146, 148, 149, 150, 152, 155, 158, 225 and 226, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from SEQ ID NOs: 139, 140, 142, 143, 145, 146, 148, 149, 150, 152, 155, 158, 225 and 226.

In some embodiments, the vector comprises a polynucleotide encoding a fusion polypeptide or a compound fusion polypeptide, as described herein, which has a nucleic acid sequence of SEQ ID NO: 139, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 139. In some embodiments, the vector comprises a polynucleotide encoding a fusion polypeptide or a compound fusion polypeptide, as described herein, which has a nucleic acid sequence of SEQ ID NO: 142, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 142. In some embodiments, the vector comprises a polynucleotide encoding a fusion polypeptide or a compound fusion polypeptide, as described herein, which has a nucleic acid sequence of SEQ ID NO: 145, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 145. In some embodiments, the vector comprises a polynucleotide encoding a fusion polypeptide or a compound fusion polypeptide, as described herein, which has a nucleic acid sequence of SEQ ID NO: 148, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 148. In various embodiments, the vector comprising one or more of the foregoing polynucleotides is a Lymphocytic choriomeningitis mammarenavirus (LCMV).

In some embodiments, the vector comprises a polynucleotide encoding a fusion polypeptide or a compound fusion polypeptide, as described herein, which has a nucleic acid sequence of SEQ ID NO: 150, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 150. In some embodiments, the vector comprises a polynucleotide encoding a fusion polypeptide or a compound fusion polypeptide, as described herein, which has a nucleic acid sequence of SEQ ID NO: 152, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 152. In some embodiments, the vector comprises a polynucleotide encoding a fusion polypeptide or a compound fusion polypeptide, as described herein, which has a nucleic acid sequence of SEQ ID NO: 155, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 155. In some embodiments, the vector comprises a polynucleotide encoding a fusion polypeptide or a compound fusion polypeptide, as described herein, which has a nucleic acid sequence of SEQ ID NO: 158, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 158. In various embodiments, the vector comprising one or more of the foregoing polynucleotides is a Cali mammarenavirus (a.k.a., Pichinde mammarenavirus or Pichinde arenavirus).

In some embodiments, the vector comprises a polynucleotide encoding a fusion polypeptide or a compound fusion polypeptide, as described herein, which has a nucleic acid sequence of SEQ ID NO: 225, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 225. In some embodiments, the vector comprises a polynucleotide encoding a fusion polypeptide or a compound fusion polypeptide, as described herein, which has a nucleic acid sequence of SEQ ID NO: 226, or a nucleic acid sequence that is at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence of SEQ ID NO: 226.

In various embodiments, the vector comprises the following first polynucleotide and second polynucleotide: SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively;
SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively;
SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively;
SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively;
SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively;
SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively;
SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively;
SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively;
SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively;
SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively;
SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively;
SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively;
SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively;
SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively;
SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively;
SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively;
SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively;
SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively;
SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively;
SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively;
SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively;
SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively;
SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively;
SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively;
SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively;
SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively;
SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively;
SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively;
SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively;
SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively;
SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively;
SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively;
SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively;
SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively;
SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively;
SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or
SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively.

In various embodiments, the vector comprises the following first polynucleotide and second polynucleotide:
a) a first polynucleotide comprising SEQ ID NO: 130 or SEQ ID NO: 131, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 130 or SEQ ID NO: 131, respectively, and
b) a second polynucleotide comprising SEQ ID NO: 134 or SEQ ID NO: 135, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 134 or SEQ ID NO: 135.

In various embodiments, the vector comprises the following first polynucleotide and second polynucleotide:
a) a first polynucleotide comprising SEQ ID NO: 132 or SEQ ID NO: 133, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 132 or SEQ ID NO: 133, respectively, and
b) a second polynucleotide comprising SEQ ID NO: 136 or SEQ ID NO: 137, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 136 or SEQ ID NO: 137.

In various embodiments, the vector comprises the following first polynucleotide and second polynucleotide:
a) a first polynucleotide comprising SEQ ID NO: 139, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 139, respectively, and
b) a second polynucleotide comprising SEQ ID NO: 145, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 145.

In various embodiments, the vector comprises the following first polynucleotide and second polynucleotide:
a) a first polynucleotide comprising SEQ ID NO: 140, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 140, respectively, and
b) a second polynucleotide comprising SEQ ID NO: 146, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 146.

a) a first polynucleotide comprising SEQ ID NO: 142, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 142, respectively, and
b) a second polynucleotide comprising SEQ ID NO: 148, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 148.

In various embodiments, the vector comprises the following first polynucleotide and second polynucleotide:
a) a first polynucleotide comprising SEQ ID NO: 143, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 143, respectively, and
b) a second polynucleotide comprising SEQ ID NO: 149, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 149.

In various embodiments, the vector comprises the following first polynucleotide and second polynucleotide:
a) a first polynucleotide comprising SEQ ID NO: 150, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 150, respectively, and
b) a second polynucleotide comprising SEQ ID NO: 152, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 152.

In various embodiments, the vector comprises the following first polynucleotide and second polynucleotide:
a) a first polynucleotide comprising SEQ ID NO: 151, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 151, respectively, and
b) a second polynucleotide comprising SEQ ID NO: 153, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 153.
a) a first polynucleotide comprising SEQ ID NO: 154, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 154, respectively, and
b) a second polynucleotide comprising SEQ ID NO: 157, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 157.

In various embodiments, the vector comprises the following first polynucleotide and second polynucleotide:
a) a first polynucleotide comprising SEQ ID NO: 155, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 155, respectively, and
b) a second polynucleotide comprising SEQ ID NO: 158, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 158.

In various embodiments, the vector comprises the following first polynucleotide and second polynucleotide:
a) a first polynucleotide comprising SEQ ID NO: 156, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 156, respectively, and
b) a second polynucleotide comprising SEQ ID NO: 159, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 159.

In various embodiments, the vector comprises the following first polynucleotide and second polynucleotide:
a) a first polynucleotide comprising SEQ ID NO: 160, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 160, respectively, and
b) a second polynucleotide comprising SEQ ID NO: 161, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 161.
a) a first polynucleotide comprising SEQ ID NO: 162, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 162, respectively, and
b) a second polynucleotide comprising SEQ ID NO: 163, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 163.

In various embodiments, the vector comprises the following first polynucleotide and second polynucleotide:
a) a first polynucleotide comprising SEQ ID NO: 164, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 164, respectively, and
b) a second polynucleotide comprising SEQ ID NO: 165, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 165.

In various embodiments, the vector comprises the following first polynucleotide and second polynucleotide:
a) a first polynucleotide comprising SEQ ID NO: 166, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 166, respectively, and
b) a second polynucleotide comprising SEQ ID NO: 167, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 167.

In various embodiments, the vector comprises the following first polynucleotide and second polynucleotide:
a) a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and
b) a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211.
a) a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and
b) a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213.

In various embodiments, the vector comprises the following first polynucleotide and second polynucleotide:
a) a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and
b) a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215.

In various embodiments, the vector comprises the following first polynucleotide and second polynucleotide:
a) a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and
b) a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217.

In various embodiments, the vector comprises the following first polynucleotide and second polynucleotide:
a) a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and
b) a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219.
a) a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and
b) a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In various embodiments, the vector comprises the following first polynucleotide and second polynucleotide:
a) a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and
b) a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In various embodiments, the vector comprises a polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225.

In various embodiments, the vector comprises a polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In other embodiments, the vector that is used is pcDNATM3.1+ (ThermoFisher, MA).

In some embodiments, the vector is viral vector. As appropriate, the viral vector can be a DNA virus or an RNA virus, including a self-replicating RNA virus. Self-replicating RNA viruses include Alphaviruses, and are described, e.g., in Lundstrom, Molecules. (2018) 23 (12). pii: E3310 (PMID: 30551668); and Ljungberg, et al., Expert Rev Vaccines. (2015) 14 (2): 177-94). Additional alphaviruses of use as viral vectors are described, e.g., in WO 2020/097393 and WO 2018/208856. In various embodiments, the viral vector is from a virus selected from adenovirus, adeno-associated virus, arenavirus, alphavirus, self-replicating alphavirus, poxvirus, cytomegalovirus, rhabdovirus, vesicular stomatitis virus, flavivirus, maraba virus and vaccinia virus. In some embodiments, the viral vector is from a viral family selected from: Adenoviridae (e.g., Adenovirus, adeno-associated virus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Cytomegalovirus, Herpesvirus, e.g., HSV-1), Parvoviridae (e.g., Parvovirus H1), Poxviridae (e.g. Vaccinia virus, e.g. modified vaccinia Ankara (MVA)), Flaviviridae (e.g. Yellow fever virus), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesiculovirus, including Maraba vesiculovirus and Vesicular stomatitis virus (VSV)), Togaviridae (e.g., Alphavirus, e.g., Venezuelan equine encephalitis virus, e.g., self-replicating Alphavirus; Sindbis virus), Enteroviridae (e.g., Echovirus). Illustrative modified vaccinia viral vectors of use for expressing the present fusion polypeptides and/or compound fusion polypeptides are described, e.g., in WO 2019/134049.

In some embodiments, the viral expression vector is an arenavirus vector selected from Lymphocytic choriomeningitis mammarenavirus (LCMV) (NCBI: txid11623), Cali mammarenavirus (a.k.a., Pichinde mammarenavirus or Pichinde arenavirus) (NCBI: txid2169993), Guanarito virus (GTOV) (NCBI: txid45219), Argentinian mammarenavirus (a.k.a., Junin virus (JUNV)) (NCBI: txid2169991), Lassa virus (LASV) (NCBI: txid11620), Lujo virus (LUJV) (NCBI: txid649188), Machupo virus (MACV) (NCBI: txid11628), Brazilian mammarenavirus (a.k.a., Sabia virus (SABV)) (NCBI: txid2169992), and Whitewater Arroyo virus (WWAV) (NCBI: txid46919). In some embodiments, the viral expression vector is an arenavirus vector selected from Lymphocytic choriomeningitis mammarenavirus (LCMV) or Cali mammarenavirus (a.k.a., Pichinde mammarenavirus or Pichinde arenavirus). Illustrative arenavirus vectors that can be used as delivery and expression vehicles for the herein described fusion polypeptides are described, e.g., in WO 2009/083210; WO 2015/183895; WO 2016/075250; WO 2017/198726; and U.S. Pat. No. 9,943,585.

In some embodiments, the viral expression vector is an adenovirus vector, e.g., from a human adenovirus or a simian adenovirus (e.g., a chimpanzee adenovirus, a gorilla adenovirus or a rhesus monkey adenovirus). In various embodiments, the adenovirus vector is selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (e.g. ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), ChAd147 (AdC147)), gorilla adenovirus (e.g. GC44, GC45, GC46) and rhesus adenovirus (e.g., RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66). Illustrative Chimpanzee, Gorilla and Rhesus monkey adenovirus vectors that can be used as delivery and expression vehicles for the herein described fusion polypeptides and/or compound fusion polypeptides are described, e.g., in WO 2019/076880; WO 2019/076877; Andrabi et al., (2019) Cell Reports 27:2426-2441; Guo, et al., Hum Vaccin Immunother. (2018) 14 (7): 1679-1685; Abbink, et al., J Virol. (2015) 89 (3): 1512-22; Abbink, et al., J Virol. (2018) 92 (6). pii: e01924-17, and in WO 2020/243719A1 and WO 2018/098362A1.

In various embodiments, the viral expression vector is incapable of replication (i.e., replication defective or replication deficient), has reduced or diminished capacity for replication, e.g., in comparison to a wild-type viral vector (i.e., replication attenuated) or is replication competent.

Further provided are host cells comprising one or more polynucleotides encoding one or more of the fusion polypeptides or compound fusion polypeptides, or one or more vectors expressing the fusion polypeptides or compound fusion polypeptides, as described herein. Any of a variety of host cells can be used. In one embodiment, a host cell is a prokaryotic cell, for example, E. coli. In another embodiment, a host cell is a eukaryotic cell, for example, a yeast cell, a plant cell, an insect cell, a mammalian cell, such as a Chinese Hamster Ovary (CHO)-based or CHO-origin cell line (e.g., CHO-S, CHO DG44, ExpiCHO™, CHOZN® ZFN-modified GS-/-CHO cell line, CHO-K1, CHO-Kla), COS cells, BHK cells, NSO cells or Bowes melanoma cells. Examples of human host cells are, inter alia, HeLa, 911, AT1080, A549 and HEK293 (e.g., HEK293E, HEK293F, HEK293H, HEK293T, Expi293TM). In addition, the fusion polypeptides and/or compound fusion polypeptides can be expressed in a yeast cell such as Pichia (see, e.g., Powers et al., J Immunol Methods. 251:123-35 (2001)), Hanseula, or Saccharomyces.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with one or more polynucleotides encoding one or more fusion polypeptides or compound fusion polypeptides, as described herein. As appropriate, the host cells can be infected with one or more vectors expressing one or more fusion polypeptides or compound fusion polypeptides, as described herein. In some embodiments, the host cells are capable of being infected with and propagating one or more replication attenuated or replication competent vectors expressing one or more fusion polypeptides or compound fusion polypeptides, as described herein. Illustrative cells useful for infecting with and/or propagating viral vectors include without limitation BHK-21, A549, Vero and HEK293 (e.g., HEK293E, HEK293F, HEK293H, HEK293T, Expi293TM) cells. In certain embodiments, the host cells express the Coxsackievirus and adenovirus receptor (CAR), e.g., MDCK, Caco-2 or Calu-3 host cells. In certain embodiments, the polynucleotides integrate into the genome of the host cell.

### 5. Pharmaceutical Compositions/Immunogenic Compositions

Provided are pharmaceutical compositions or immunogenic compositions comprising one or more of the fusion polypeptides or compound fusion polypeptides, as described herein, or a polynucleotide encoding one or more of the fusion polypeptides or compound fusion polypeptides, as described herein, or a viral expression vector comprising one or more of such polynucleotides, and a pharmaceutically acceptable diluent, carrier or excipient. Generally, the pharmaceutical compositions described herein are immunogenic. In certain embodiments, the pharmaceutical composition comprises a therapeutically effective amount of the one or more fusion polypeptides or compound fusion polypeptides, or one or more polynucleotides encoding one or more of the fusion polypeptides or compound fusion polypeptides, or one or more viral expression vectors containing one or more of the polynucleotides encoding one or more of the fusion polypeptides or compound fusion polypeptides.

Various pharmaceutically acceptable diluents, carriers, and excipients, and techniques for the preparation and use of pharmaceutical compositions or immunogenic compositions will be known to those of skill in the art in light of the present disclosure. Illustrative pharmaceutical compositions/immunogenic compositions and pharmaceutically acceptable diluents, carriers, and excipients are also described in, e.g., Loyd V. Allen Jr (Editor), “Remington: The Science and Practice of Pharmacy,” 22nd Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing.

In certain embodiments, the polynucleotides or vectors are formulated into a lipoplex, e.g., a lipid nanoparticle (LNP). As used herein, a “lipoplex” refers to cationic liposomes that are nonviral (synthetic) lipid carriers of one or more polynucleotides, e.g., RNA, DNA. For example, in some embodiments where the fusion polypeptides and/or compound fusion polypeptides are expressed from self-replicating or self-amplifying RNA molecules, the self-replicating or self-amplifying RNA can be formulated into LNPs. As used herein, the term “lipid nanoparticle” refers to one or more spherical nanoparticles with an average diameter of between about 10 to about 1000 nanometers, and which comprise a solid lipid core matrix that can solubilize lipophilic molecules. In certain embodiments, the lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, each of which is incorporated by reference in its entirety. In one embodiment, a self-replicating or self-amplifying RNA (saRNA) molecule encoding one or more of the fusion polypeptides or compound fusion polypeptides described herein is formulated or condensed into polyethylenimine (PEI)-polyplex delivery vehicles, e.g., as described in Moyo, et al., Mol Ther Methods Clin Dev. (2018) 12:32-46; Blakney, et al., Gene Therapy (2019) 26:363-372; Démoulins, et al., Nanomedicine. (2016) Apr; 12 (3): 711-722 and Démoulins, et al., J Control Release. (2017) 266:256-271, which can be nanoparticulate.

In embodiments where the fusion polypeptides or compound fusion polypeptides are expressed from a viral expression vector, the viral expression vector can be formulated for the desired route of administration, e.g., as an isotonic pharmaceutically acceptable aqueous solution for intravenous, intramuscular, subcutaneous, intradermal or intranodal administration. In some embodiments, the viral expression vector can be formulated for mucosal, e.g., buccal or intra-rectal delivery. Illustrative formulations for viral expression vectors that can be used in the herein described pharmaceutical compositions/immunogenic compositions and methods are described, e.g., in Manfredsson and Benskey, editors, “Viral Vectors for Gene Therapy: Methods and Protocols (Methods in Molecular Biology),” 2019, Book 1937 in Methods in Molecular Biology Series, Humana Press; WO 2017/013169 (formulation of Adenoviral vectors in an aqueous mixture or freeze dried composition in the presence of amorphous sugar and low salt concentration); and Kumru, et al., J Pharm Sci. (2018) Nov; 107 (11): 2764-3374 (aqueous formulations buffered in Tris and containing proline, lactose, and mannitol as stabilizing additives). Formulation of arenavirus vectors is described, e.g., in WO 2009/083210; WO 2016/075250 and WO 2017/198726. In certain embodiments, the viral expression vectors are delivered via microneedle-mediated delivery, e.g., as described in Zaric, et al., Expert Opin Drug Deliv. (2017) Oct; 14 (10): 1177-1187. Intranodal delivery of mRNA vaccines are described, e.g., in Jong, et al., Vaccines (Basel). (2019) 7 (4): 209; Leal, et al., AIDS. (2018) 32 (17): 2533-2545; de Jong, et al., Trials. (2019) 20 (1): 361; and Joe, et al., J Transl Med. (2019) 17 (1): 242.

In some embodiments, each carrier, diluent or excipient is “acceptable” in the sense of being compatible with the other ingredients of the pharmaceutical composition/immunogenic composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., a buffer having a pKa in the range of about 6.0 to about 8.0, e.g., a physiologically acceptable buffer, e.g., selected from phosphate, carbonate, bicarbonate, citrate, maleate, glycine-glycine, HEPES, HEPPSO, HEPPS, imidazole, BICINE, TRICINE, Tris, and BIS-Tris; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Hank's solution, Ringer's solution; ethyl alcohol; phosphate buffer solutions; amino acids (e.g., charged amino acids, including without limitation, aspartate, asparagine, glutamate, glutamine, histidine, arginine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In one particular formulation, an arenavirus vector (e.g., a LCMV or Pichinde mammarenavirus vector (PICV)) described herein is formulated in an isotonic aqueous solution comprising a biologically compatible buffer having a pKa in the range of about 6.0 to about 8.0 (e.g., HEPES and NaCl), at a neutral or near-neutral pH and a non-ionic surfactant (e.g., PLURONIC® F68 (a.k.a., poloxamer 188)). In one particular formulation, an arenavirus vector (e.g., a LCMV or Pichinde mammarenavirus vector) described herein is formulated in an isotonic aqueous solution comprising HEPES buffer at pH 7.4, NaCl, and PLURONIC® F68 (a.k.a., poloxamer 188). Schleiss, et al. (Clin Vaccine Immunol. 2017 Jan. 5; 24 (1): e00300-16) describes an LCMV formulating LCMV vectors in a diluent of 25 mM HEPES, 150 mM NaCl, 0.01% PLURONIC® F68; pH 7.4), which can be used to formulate the herein described arenavirus vectors. A final concentration of 10% sorbitol was added before freezing below −60° C.

The formulation of and delivery methods of pharmaceutical compositions or immunogenic compositions will generally be adapted according to the site and the disease to be treated. Exemplary formulations include without limitation, those suitable for parenteral administration, e.g., intravenous, intra-arterial, intramuscular, subcutaneous or intranodal administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. In some embodiments, the pharmaceutical compositions or immunogenic compositions are formulated for parenteral, e.g., intravenous, subcutaneous, intranodal or oral administration. In some embodiments, the pharmaceutical compositions or immunogenic compositions are formulated for mucosal, e.g., buccal, intrarectal and/or intravaginal administration. In some embodiments, for intrarectal administration, the pharmaceutical composition or immunogenic composition can be formulated as a suppository or as an enema. In some embodiments, for intravaginal administration, the pharmaceutical composition or immunogenic composition can be formulated as a pessary.

In certain embodiments, pharmaceutical compositions/immunogenic compositions are sterile. In certain embodiments, the pharmaceutical composition or immunogenic composition has a pH in the range of 4.5 to 8.5, 4.5 to 6.5, 6.5 to 8.5, or a pH of about 5.0, about 5.5, about 6.0, about 6.5, about 7.0, about 7.5, about 8.0 or about 8.5. In one embodiment, the pharmaceutical composition/immunogenic composition has an osmolarity in the range of 240-260 or 250-330 mOsmol/L. In certain embodiments, the pharmaceutical composition/immunogenic composition is isotonic or near isotonic.

In some embodiments, the pharmaceutical compositions or immunogenic compositions are liquids or solids. In some embodiments, the pharmaceutical composition or immunogenic composition comprises an aqueous solution. In some embodiments, the pharmaceutical composition or immunogenic composition is lyophilized or is a frozen liquid.

In some embodiments, the pharmaceutical composition or immunogenic composition further comprises one or more additional therapeutic agents, e.g., a second therapeutic agent, or second and third therapeutic agents, for use in combination therapies, as described herein.

In certain embodiments, the pharmaceutical composition or immunogenic composition further comprises an adjuvant. Illustrative adjuvants that can be co-formulated or co-administered with the herein described fusion polypeptides or compound fusion polypeptides, polynucleotides encoding such fusion polypeptides or compound fusion polypeptides, and vectors expressing such fusion polypeptides or compound fusion polypeptides, include without limitation cytokines, chemokines, immune costimulatory molecules, toll-like receptor agonists, second mitochondria-derived activator of caspases (SMAC) mimetics or inhibitors of immune suppressive pathways (e.g., immune checkpoint inhibitors), as described herein, and in Li, et al., Curr Issues Mol Biol. (2017) 22:17-40. Other adjuvants that can be co-formulated or co-administered with the herein described fusion polypeptides or compound fusion polypeptides, polynucleotides encoding such fusion polypeptides or compound fusion polypeptides, and vectors expressing such fusion polypeptides or compound fusion polypeptides, include without limitation mineral salts (e.g., aluminum salts (e.g., alum), calcium phosphate, incomplete Freunds's adjuvant), lipid particles (e.g., MF59, cochleates, virus-like particles), microparticles (e.g., virosomes, polylactic acid (PLA), poly [lactide-coglycolide] (PLG)), immune potentiators (e.g., dsRNA: Poly (I: C), Poly-IC: LC, Monophosphoryl lipid A (MPL), LPS, Flagellin, Imidazoquinolines: imiquimod (R837), resiquimod (848), CpG oligodeoxynucleotides (ODN), Muramyl dipeptide (MDP), Saponins (QS-21)), and mucosal adjuvants (e.g., Cholera toxin (CT), Heat-labile enterotoxin (LTK3 and LTR72), Chitosan). Adjuvants that can be co-formulated or co-administered with the herein described fusion polypeptides, polynucleotides encoding such fusion polypeptides and vectors expressing such fusion polypeptides are summarized in Apostolico, et al., J Immunol Res. (2016) 2016:1459394.

In some embodiments, the pharmaceutical compositions or immunogenic compositions comprise two or more fusion polypeptides and/or compound fusion polypeptides, two or more polynucleotides encoding such fusion polypeptides and/or compound fusion polypeptides, or two or more vectors expressing such fusion polypeptides and/or compound fusion polypeptides. In various embodiments, the pharmaceutical compositions or immunogenic compositions comprise a first fusion polypeptide and a second fusion polypeptide that are bivalent, or one or more vectors comprising one or more polynucleotides encoding the bivalent first and second fusion polypeptides. In some embodiments, the pharmaceutical compositions or immunogenic compositions comprise a first fusion polypeptide and a second fusion polypeptide, or one or more vectors comprising one or more polynucleotides encoding the first and second fusion polypeptides, the first and second polypeptides comprising the following polypeptide segments, in sequential order, from N-terminus to C terminus, optionally joined or connected by one or more linkers: SEQ ID NOs: 6, 4, 16 and 26, and SEQ ID NOs: 7, 5, 27 and 17;
SEQ ID NOs: 12, 24, 8 and 10, and SEQ ID NOs: 9, 25, 13 and 11;
SEQ ID NOs: 14, 22, 18, 24 and 20, and SEQ ID NOs: 15, 25, 19, 23 and 21;
SEQ ID NOs: 26, 16, 4 and 6, and SEQ ID NOs: 7, 27, 5 and 17;
SEQ ID NOs: 8, 24, 12 and 10, and SEQ ID NOs: 13, 25, 9 and 11;
SEQ ID NOs: 22, 24, 14, 18 and 20, and SEQ ID NOs: 25, 23, 15, 21 and 19;
SEQ ID NOs: 20, 6, 4, 18, 16 and 26, and SEQ ID NOs: 7, 19, 5, 21, 27 and 17;
SEQ ID NOs: 8, 24, 14, 12, 22 and 10, and SEQ ID NOs: 9, 13, 25, 23, 15 and 11;
SEQ ID NOs: 18, 26, 20, 4, 6 and 16, and SEQ ID NOs: 7, 21, 17, 5, 27 and 19;
SEQ ID NOs: 22, 24, 12, 14, 8 and 10, and SEQ ID NOs: 15, 25, 9, 23, 13 and 11;
SEQ ID NOs: 24, 6, 4, 20, 18 and 32, and SEQ ID NOs: 8, 30, 14, 12, 26 and 10;
SEQ ID NOs: 24, 6, 4, 20, 18 and 32, and SEQ ID NOs: 7, 21, 5, 25, 33 and 19;
SEQ ID NOs: 24, 6, 4, 20, 18 and 32, and SEQ ID NOs: 8, 30, 14, 12, 26 and 10;
SEQ ID NOs: 8, 30, 14, 12, 26 and 10, and SEQ ID NOs: 9, 13, 31, 27, 15 and 11;
SEQ ID NOs: 6, 20, 4, 24, 32 and 18 and SEQ ID NOs: 8, 12, 30, 26, 14 and 10;
SEQ ID NOs: 7, 21, 5, 25, 33 and 19 and SEQ ID NOs: 9, 13, 31, 27, 15 and 11;
SEQ ID NOs: 20, 32, 24, 4, 6 and 18, and SEQ ID NOs: 26, 30, 12, 14, 8 and 10;
SEQ ID NOs: 7, 25, 19, 5, 33 and 21, and SEQ ID NOs: 15, 31, 9, 27, 13 and 11;
SEQ ID NOs: 24, 6, 16 and 18, and SEQ ID NOs: 26, 20, 10 and 28;
SEQ ID NOs: 24, 6, 16 and 18, and SEQ ID NOs: 26, 10, 20 and 28;
SEQ ID NOs: 24, 6, 16 and 18, and SEQ ID NOs: 7, 25, 17 and 19;
SEQ ID NOs: 7, 19, 17 and 25, and SEQ ID NOs: 21, 27, 11 and 29;
SEQ ID NOs: 24, 16, 6 and 18, and SEQ ID NOs: 27, 11, 21 and 29;
SEQ ID NOs: 7, 25, 17 and 19, and SEQ ID NOs: 11, 27, 21 and 29;
SEQ ID NOs: 7, 25, 17 and 19, and SEQ ID NOs: 21, 27, 11 and 29;
SEQ ID NOs: 22, 6, 20 and 28, and SEQ ID NOs: 23, 7, 21 and 29;
SEQ ID NOs: 22, 20, 6 and 28, and SEQ ID NOs: 7, 21, 23 and 29;
SEQ ID NOs: 26, 10, 20 and 28, and SEQ ID NOs: 21, 27, 11 and 29;
SEQ ID NOs: 22, 6, 16, 20, 18, 28, 26 and 10; or.
SEQ ID NOs: 7, 21, 19, 17, 27, 25, 29 and 11.

In some embodiments, the pharmaceutical composition or immunogenic composition comprises one or more, e.g., two or more, fusion polypeptides, or one or more, e.g., two or more, vectors comprising one or more polynucleotides encoding one or more, e.g., two or more, fusion polypeptides, comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the pharmaceutical composition or immunogenic composition comprises one or more, e.g., two or more, fusion polypeptides, or one or more, e.g., two or more, vectors comprising one or more polynucleotides encoding one or more, e.g., two or more, fusion polypeptides, comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223.

In some embodiments, the pharmaceutical compositions or immunogenic compositions comprise the following first fusion polypeptide and second fusion polypeptide, or one or more vectors comprising one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223. In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of SEQ ID NO: 209, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209. In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of SEQ ID NO: 222, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222. In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of SEQ ID NO: 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223. In some embodiments, the pharmaceutical compositions or immunogenic compositions comprise a compound fusion polypeptide or a vector comprising a polynucleotide encoding a compound fusion polypeptide, the compound fusion polypeptide comprising or consisting of an amino acid sequence of SEQ ID NO: 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 227.

In some embodiments, the pharmaceutical compositions or immunogenic compositions comprise the following first fusion polypeptide and second fusion polypeptide, or one or more vectors comprising one or more polynucleotides encoding the following first fusion polypeptide and second fusion polypeptide, in sequential order, from N-terminus to C-terminus, optionally joined or connected by one or more linkers: SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively;
SEQ ID NOs: 76 and 77, or fusion polypeptides that are at least 80%, 81%, 82%, 97%, 98% or 99% identical to any one of SEQ ID NOs: 76 and 77, respectively;
SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 87 and 86, or fusion polypeptides that are at least 80%, 81%, 82%, 97%, 98% or 99% identical to any one of SEQ ID NOs: 87 and 86, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively;
SEQ ID NOs: 85 and 87, or fusion polypeptides that are at least 80%, 81%, 82%, 97%, 98% or 99% identical to any one of SEQ ID NOs: 85 and 87, respectively;
SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 91 and 90, or fusion polypeptides that are at least 80%, 81%, 82%, 97%, 98% or 99% identical to any one of SEQ ID NOs: 91 and 90, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively;
SEQ ID NOs: 220 and 221, or fusion polypeptides that are at least 80%, 81%, 82%, 97%, 98% or 99% identical to any one of SEQ ID NOs: 220 and 221, respectively;
SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively;
SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively;
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or
SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively, and
b) a second viral vector comprising one or more polynucleotides encoding a first fusion polypeptide and a second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively, and
b) a second viral vector comprising one or more polynucleotides encoding a first fusion polypeptide and a second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively, and
b) a second viral vector comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively, and
b) a second viral vector comprising one or more polynucleotides encoding a first fusion polypeptide and a second fusion polypeptide comprising SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively, and
b) a second viral vector comprising one or more polynucleotides encoding a first fusion polypeptide and a second fusion polypeptide comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively, and
b) a second viral vector comprising one or more polynucleotides encoding a first fusion polypeptide and a second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively.

In various embodiments, the immunogenic composition or pharmaceutical composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively.

In various embodiments, the immunogenic composition or pharmaceutical composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively.

In various embodiments, the immunogenic composition or pharmaceutical composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In various embodiments, the immunogenic composition or pharmaceutical composition comprises a viral vector or a lipoplex (e.g., LNP) comprising one or more polynucleotides encoding a first fusion polypeptide and a second fusion polypeptide, optionally joined or connected by one or more linkers, comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively.

In various embodiments, the immunogenic composition or pharmaceutical composition comprises a viral vector comprising one or more polynucleotides encoding the following first fusion polypeptide and second fusion polypeptide, optionally joined or connected by one or more linkers, SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively.

In various embodiments, the immunogenic composition or pharmaceutical composition comprises a viral vector comprising one or more polynucleotides encoding the following first fusion polypeptide and second fusion polypeptide, optionally joined or connected by one or more linkers, SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) the first viral vector comprising one or more polynucleotides encoding a first fusion polypeptide and a second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and
b) the second viral vector comprising one or more polynucleotides encoding a first fusion polypeptide and a second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) the first viral vector comprising one or more polynucleotides encoding a first fusion polypeptide and a second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and
b) the second viral vector comprising one or more polynucleotides encoding a first fusion polypeptide and a second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In various embodiments, the immunogenic composition or pharmaceutical composition comprises a viral vector comprising one or more polynucleotides encoding a first fusion polypeptide and a second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively.

In various embodiments, the immunogenic composition or pharmaceutical composition comprises a viral vector comprising one or more polynucleotides encoding a first fusion polypeptide and a second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) the first viral vector comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and
b) the second viral vector comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In various embodiments, the immunogenic composition or pharmaceutical composition comprises first and second viral vectors comprising one or more polynucleotides encoding the following first fusion polypeptide and second fusion polypeptide, optionally joined or connected by one or more linkers:
a) the first viral vector comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and
b) the second viral vector comprising one or more polynucleotides encoding a first fusion polypeptide and a second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively.

In various embodiments, the immunogenic composition or pharmaceutical composition comprises a viral vector comprising one or more polynucleotides encoding a first fusion polypeptide and a second fusion polypeptide comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively.

In various embodiments, the immunogenic composition or pharmaceutical composition comprises a viral vector comprising one or more polynucleotides encoding a first fusion polypeptide and a second fusion polypeptide comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In various embodiments, the immunogenic composition or pharmaceutical composition comprises a viral vector comprising a polynucleotide encoding a compound fusion polypeptide comprising SEQ ID NO: 105 or 206, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105 or 206.

In various embodiments, the immunogenic composition or pharmaceutical composition comprises a viral vector comprising a polynucleotide encoding a compound fusion polypeptide comprising SEQ ID NO: 107 or 207, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107 or 207.

In various embodiments, the immunogenic composition or pharmaceutical composition comprises a viral vector comprising a polynucleotide encoding a compound fusion polypeptide comprising SEQ ID NO: 109, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 109.

In various embodiments, the immunogenic composition or pharmaceutical composition comprises a viral vector comprising a polynucleotide encoding a compound fusion polypeptide comprising SEQ ID NO: 111, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 111.

In various embodiments, the immunogenic composition or pharmaceutical composition comprises a viral vector comprising a polynucleotide encoding a compound fusion polypeptide comprising SEQ ID NO: 200, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200.

In various embodiments, the immunogenic composition or pharmaceutical composition comprises a viral vector comprising a polynucleotide encoding a compound fusion polypeptide comprising SEQ ID NO: 201, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201.

In various embodiments, the immunogenic composition or pharmaceutical composition comprises a viral vector comprising a polynucleotide encoding a compound fusion polypeptide comprising SEQ ID NO: 202, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202.

In various embodiments, the immunogenic composition or pharmaceutical composition comprises a viral vector comprising a polynucleotide encoding a compound fusion polypeptide comprising SEQ ID NO: 203, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203.

In various embodiments, the immunogenic composition or pharmaceutical composition comprises a viral vector comprising a polynucleotide encoding a compound fusion polypeptide comprising SEQ ID NO: 204, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204.

In various embodiments, the immunogenic composition or pharmaceutical composition comprises a viral vector comprising a polynucleotide encoding a compound fusion polypeptide comprising SEQ ID NO: 205, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205.

In various embodiments, the immunogenic composition or pharmaceutical composition comprises a viral vector comprising a polynucleotide encoding a compound fusion polypeptide comprising SEQ ID NO: 208, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208.

In various embodiments, the immunogenic composition or pharmaceutical composition comprises a viral vector comprising a polynucleotide encoding a compound fusion polypeptide comprising SEQ ID NO: 209, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209.

In various embodiments, the immunogenic composition or pharmaceutical composition comprises a viral vector comprising a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222.

In various embodiments, the immunogenic composition or pharmaceutical composition comprises a viral vector comprising a polynucleotide encoding a compound fusion polypeptide comprising SEQ ID NO: 223, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223.

In various embodiments, the immunogenic composition or pharmaceutical composition comprises a viral vector comprising a polynucleotide encoding a compound fusion polypeptide comprising SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 227.

In various embodiments, the immunogenic composition or pharmaceutical composition comprises a first viral vector or a lipoplex (e.g., LNP) comprising a first polynucleotide encoding a first polypeptide comprising SEQ ID NO: 200, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, and a second viral vector or a lipoplex (e.g., LNP) comprising a second polynucleotide encoding a second polypeptide comprising SEQ ID NO: 201, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201.

In various embodiments, the immunogenic composition or pharmaceutical composition comprises a first viral vector or a lipoplex (e.g., LNP) comprising a first polynucleotide encoding a first polypeptide comprising SEQ ID NO: 202, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202, and a second viral vector or a lipoplex (e.g., LNP) comprising a second polynucleotide encoding a second polypeptide comprising SEQ ID NO: 203, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203.

In various embodiments, the immunogenic composition or pharmaceutical composition comprises a first viral vector or a lipoplex (e.g., LNP) comprising a first polynucleotide encoding a first polypeptide comprising SEQ ID NO: 204, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204, and a second viral vector or a lipoplex (e.g., LNP) comprising a second polynucleotide encoding a second polypeptide comprising SEQ ID NO: 205, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205.

In various embodiments, the immunogenic composition or pharmaceutical composition comprises a first viral vector or a lipoplex (e.g., LNP) comprising a first polynucleotide encoding a first polypeptide comprising SEQ ID NO: 105, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, and a second viral vector or a lipoplex (e.g., LNP) comprising a second polynucleotide encoding a second polypeptide comprising SEQ ID NO: 107, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107.

In various embodiments, the immunogenic composition or pharmaceutical composition comprises a first viral vector or a lipoplex (e.g., LNP) comprising a first polynucleotide encoding a first polypeptide comprising SEQ ID NO: 206, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, and a second viral vector or a lipoplex (e.g., LNP) comprising a second polynucleotide encoding a second polypeptide comprising SEQ ID NO: 207, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207.

In various embodiments, the immunogenic composition or pharmaceutical composition comprises a first viral vector or a lipoplex (e.g., LNP) comprising a first polynucleotide encoding a first polypeptide comprising SEQ ID NO: 208, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, and a second viral vector or a lipoplex (e.g., LNP) comprising a second polynucleotide encoding a second polypeptide comprising SEQ ID NO: 209 or SEQ ID NO: 227, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In various embodiments, the immunogenic composition or pharmaceutical composition comprises a first viral vector or a lipoplex (e.g., LNP) comprising a first polynucleotide encoding a first polypeptide comprising SEQ ID NO: 222, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and a second viral vector or a lipoplex (e.g., LNP) comprising a second polynucleotide encoding a second polypeptide comprising SEQ ID NO: 209 or SEQ ID NO: 227, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In various embodiments, the immunogenic composition or pharmaceutical composition comprises a first viral vector or a lipoplex (e.g., LNP) comprising a first polynucleotide encoding a first polypeptide comprising SEQ ID NO: 222, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and a second viral vector or a lipoplex (e.g., LNP) comprising a second polynucleotide encoding a second polypeptide comprising SEQ ID NO: 223, or a polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223.

In some embodiments, the pharmaceutical compositions or immunogenic compositions comprise a polynucleotide comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the pharmaceutical compositions or immunogenic compositions comprise a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226.

In some embodiments, the pharmaceutical compositions or immunogenic compositions comprise the following first polynucleotide and second polynucleotide, or one or more vectors comprising the following first polynucleotide and second polynucleotide, the first and second polynucleotides comprising or consisting of, respectively:
SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively;
SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively;
SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively;
SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively;
SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively;
SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively;
SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively;
SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively;
SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively;
SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively;
SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively;
SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively;
SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively;
SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively;
SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively;
SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively;
SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively;
SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively;
SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively;
SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively;
SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively;
SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively;
SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively;
SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively;
SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively;
SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively;
SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively;
SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively;
SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively;
SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively;
SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively;
SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively;
SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively;
SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively;
SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively;
SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or. SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively.

In various embodiments, the immunogenic composition or pharmaceutical composition comprises first and second viral vectors comprising one or more polynucleotides:
a) the first viral vector comprising first and second polynucleotides of SEQ ID NOs: 131 and 135, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively, and
b) the second viral vector comprising first and second polynucleotides of SEQ ID NOs: 133 and 137, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively.

In various embodiments, the immunogenic composition or pharmaceutical composition comprises first and second viral vectors comprising one or more polynucleotides:
a) the first viral vector comprising first and second polynucleotides of SEQ ID NOs: 139 and 145, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively, and
b) the second viral vector comprising first and second polynucleotides of SEQ ID NOs: 142 and 148, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively.

In various embodiments, the immunogenic composition or pharmaceutical composition comprises first and second viral vectors comprising one or more polynucleotides:
a) the first viral vector comprising first and second polynucleotides of SEQ ID NOs: 140 and 146, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively, and
b) the second viral vector comprising first and second polynucleotides of SEQ ID NOs: 143 and 149, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively.

In various embodiments, the immunogenic composition or pharmaceutical composition comprises first and second viral vectors comprising one or more polynucleotides: a) the first viral vector comprising first and second polynucleotides of SEQ ID NOs: 150 and 152, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and
b) the second viral vector comprising first and second polynucleotides of SEQ ID NOs: 154 and 157, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively.

In various embodiments, the immunogenic composition or pharmaceutical composition comprises first and second viral vectors comprising one or more polynucleotides:
a) the first viral vector comprising first and second polynucleotides of SEQ ID NOs: 150 and 152, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and
b) the second viral vector comprising first and second polynucleotides of SEQ ID NOs: 155 and 158, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively.

In various embodiments, the immunogenic composition or pharmaceutical composition comprises first and second viral vectors comprising one or more polynucleotides:
a) the first viral vector comprising first and second polynucleotides of SEQ ID NOs: 151 and 153, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively, and
b) the second viral vector comprising first and second polynucleotides of SEQ ID NOs: 156 and 159, or first and second polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively.

In various embodiments, the immunogenic composition or pharmaceutical composition comprises first and second viral vectors comprising one or more polynucleotides:
a) the first viral vector comprising a polynucleotide of SEQ ID NO: 160, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 160, respectively, and
b) the second viral vector comprising a polynucleotide of SEQ ID NO: 161, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 161, respectively.

In various embodiments, the immunogenic composition or pharmaceutical composition comprises first and second viral vectors comprising one or more polynucleotides:
a) the first viral vector comprising a polynucleotide of SEQ ID NO: 162, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 162, respectively, and
b) the second viral vector comprising a polynucleotide of SEQ ID NO: 163, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 163, respectively.

In various embodiments, the immunogenic composition or pharmaceutical composition comprises first and second viral vectors comprising one or more polynucleotides:
a) the first viral vector comprising a polynucleotide of SEQ ID NO: 164, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 164, respectively, and
b) the second viral vector comprising a polynucleotide of SEQ ID NO: 165, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 165, respectively.

In various embodiments, the immunogenic composition or pharmaceutical composition comprises first and second viral vectors comprising one or more polynucleotides:
a) the first viral vector comprising a polynucleotide of SEQ ID NO: 166, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 166, respectively, and
b) the second viral vector comprising a polynucleotide of SEQ ID NO: 167, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 167, respectively.

In various embodiments, the immunogenic composition or pharmaceutical composition comprises first and second viral vectors comprising one or more polynucleotides:
a) the first viral vector comprising a polynucleotide of SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and
b) the second viral vector comprising a polynucleotide of SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211.

In various embodiments, the immunogenic composition or pharmaceutical composition comprises first and second viral vectors comprising one or more polynucleotides:
a) the first viral vector comprising a polynucleotide of SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and
b) the second viral vector comprising a polynucleotide of SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213.

In various embodiments, the immunogenic composition or pharmaceutical composition comprises first and second viral vectors comprising one or more polynucleotides:
a) the first viral vector comprising a polynucleotide of SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and
b) the second viral vector comprising a polynucleotide of SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215.

In various embodiments, the immunogenic composition or pharmaceutical composition comprises first and second viral vectors comprising one or more polynucleotides:
a) the first viral vector comprising a polynucleotide of SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and
b) the second viral vector comprising a polynucleotide of SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217.

In various embodiments, the immunogenic composition or pharmaceutical composition comprises first and second viral vectors comprising one or more polynucleotides:
a) the first viral vector comprising a polynucleotide of SEQ ID NO: 218, or a polynucleotide that is at least 80%, 81%, 82%, 83%, 84%, 85%, 86%, 87%, 88%, 89%, 90%,
91%, 92%, 93%, 94%, 95%, 96%, 97%, 98% or 99% identical to SEQ ID NO: 218, and b) the second viral vector comprising a polynucleotide of SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219.

In various embodiments, the immunogenic composition or pharmaceutical composition comprises first and second viral vectors comprising one or more polynucleotides:
a) the first viral vector comprising a polynucleotide of SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and
b) the second viral vector comprising a polynucleotide of SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In various embodiments, the immunogenic composition or pharmaceutical composition comprises first and second viral vectors comprising one or more polynucleotides:
a) the first viral vector comprising a polynucleotide of SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and
b) the second viral vector comprising a polynucleotide of SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In various embodiments of the pharmaceutical compositions or immunogenic compositions, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In various embodiments of the pharmaceutical compositions or immunogenic compositions, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof.

### 6. Methods of Treatment

Further provided are methods for treating or preventing an HIV infection or a related disease or disorder in a subject in need thereof (e.g., a human subject), comprising providing to a subject in need thereof an effective amount of one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein. As used herein, the term “subject” refers to a mammal. The mammal can be any mammal, for example, a human, a non-human primate, a rodent (e.g., mouse, rat, guinea pig), a dog, a cat, or a domesticated animal such as a cow, a horse, a goat, a camel, a sheep or a pig. The term “patient” refers to a human subject. As used herein, the term “effective amount” in the context of the administration of a therapy to a subject refers to the amount of a therapy that achieves a desired prophylactic or therapeutic effect. The polynucleotide may be present in a vector, e.g., a viral vector, as described herein. In some embodiments, the related disease or disorder is caused by infection with HIV. In other embodiments, it is acquired immune deficiency syndrome (AIDS). In certain embodiments, the subject is a virologically suppressed HIV-infected mammal, while in other embodiments, the subject is a treatment-naïve HIV-infected mammal or a treatment experienced HIV-infected subject that is not virologically suppressed. In certain embodiments, a treatment-naïve subject has a viral load between <50 copies/mL and 10⁸ copies/ml. In certain embodiments, a virologically suppressed subject has a viral load <50 copies/ml. In another embodiment, the subject is a mammal, e.g., a human. In certain embodiments, the subject has been diagnosed with an HIV, e.g., HIV-1 or HIV-2, infection or a related disease or disorder, e.g., AIDS, or is considered at risk for developing an HIV, e.g., HIV-1 or HIV-2, infection or a related disease or disorder, e.g., AIDS. Subjects at risk for HIV-related diseases or disorders include patients who have come into contact with an infected person or who have been exposed to HIV in some other way. Administration of a prophylactic agent can occur prior to the manifestation of symptoms characteristic of HIV-related disease or disorder, such that a disease or disorder is prevented or, alternatively, delayed in its progression.

In some embodiments, the subject is chronically infected with HIV-1. In some embodiments, the subject is acutely infected with HIV-1, e.g., has an HIV-1 infection of Fiebig stage IV or earlier, e.g. Fiebig stage III, Fiebig stage II or Fiebig stage I. In some embodiments, the subject is not receiving antiretroviral therapy (ART) or ART is discontinued prior to administration of the one or more compositions. In some embodiments, ART is discontinued after one or more administrations of the compositions. In some embodiments, ART is administered concurrently with administration of one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein.

Also provided are methods for preventing or inhibiting an increase in HIV virus titer, virus replication, virus proliferation or an amount of an HIV viral DNA, HIV proviral DNA, or HIV viral protein in a subject (e.g., a human subject). In one embodiment, the method entails providing to the subject in need thereof an amount of an one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, effective to prevent an increase in HIV titer, virus replication, or an amount of an HIV protein of one or more HIV strains or isolates in the subject. In certain embodiments, the method further comprises measuring an amount of HIV viral or proviral DNA or protein at one or more time points, e.g., before and after the subject in provided with one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein. Methods and biomarkers for determining an amount of HIV viral or proviral DNA or protein in a subject are known and available in the art, and described for example, in Siliciano, J. D. et al., Curr Opin. HIV AIDS, 5 (6): 491-7 (2010), and Rouzioux, C. et al., Curr Opin HIV AIDS, 8 (3): 170-5 (2013).

In some embodiments, one or more fusion polypeptides or compound fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, may be used in, for example, methods of inhibiting certain viruses such as HIV isolates described herein, prophylactic inhibiting or preventing infections of certain viruses such as HIV isolates described herein, detection of certain viruses such as HIV isolates described herein in a sample, inhibiting certain viruses such as HIV isolates described herein, or diagnosis of certain viruses such as HIV isolates described herein.

For in vivo treatment of mammalian subject, e.g., humans, the subject may be administered or provided a pharmaceutical composition comprising one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein. When used for in vivo therapy, the one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, are typically administered or provided to the patient in therapeutically effective amounts (i.e., amounts that eliminate or reduce the patient's viral burden and/or viral reservoir). The one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, are administered or provided to a mammalian subject, e.g., a human, in accord with known methods, such as, but not limited to, intravenous administration, e.g., as a bolus or by continuous infusion over a period of time, by intramuscular, intraperitoneal, intracerebrospinal, subcutaneous, intranodal, intraarticular, intrasynovial, intrathecal, oral, topical, or inhalation routes. The one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, may be administered parenterally, when possible, at the target cell site, or intravenously. In one embodiment, administration of the one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, to the subject is via an intravenous route. In another embodiment, administration of the one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein, to the subject is via a subcutaneous route. In additional embodiments, pharmaceutical compositions of the disclosure are administered to a subject systemically, parenterally, or locally (e.g., mucosally, including buccal, intrarectal and/or intravaginal routes).

In certain embodiments, the present disclosure provides a method for treating an HIV infection, comprising administering to a human subject in need thereof a therapeutically effective amount of one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein. In some embodiments, the present disclosure provides a method for preventing an HIV infection, comprising administering to a human subject in need thereof a therapeutically effective amount of one or more fusion polypeptides or compound fusion polypeptides, as described herein, or one or more polynucleotides encoding one or more fusion polypeptides or compound fusion polypeptides, as described herein, or one or more vectors expressing one or more fusion polypeptides or compound fusion polypeptides, as described herein.

In various embodiments, the method entails administering first and second bivalent fusion polypeptides, or first and second polynucleotides (e.g., first and second expression cassettes, first and second open reading frames) encoding the first and second fusion polypeptides, respectively, or a single viral expression vector comprising first and second polynucleotides (e.g., first and second expression cassettes, first and second open reading frames) encoding the first and second fusion polypeptides, respectively, wherein the first and second fusion polypeptides are bivalent fusion polypeptides. In certain embodiments, the single viral expression vector has a bi-segmented genome. In certain embodiments, the single viral expression vector has a tri-segmented genome. In various embodiments, the method entails administering a single compound fusion polypeptide, or a single polynucleotide (e.g., single expression cassette, single open reading frame) encoding the compound fusion polypeptide, or single viral expression vector comprising a polynucleotide encoding the compound fusion polypeptide, wherein the compound fusion polypeptide comprises bivalent fusion polypeptides.

In some embodiments, the methods entail administering to the subject: a first fusion polypeptide and a second fusion polypeptide, or one or more vectors comprising one or more polynucleotides encoding the first and second fusion polypeptides, the first and second polypeptides comprising the following polypeptide segments, in sequential order, from N-terminus to C-terminus, optionally joined or connected by one or more linkers:
SEQ ID NOs: 6, 4, 16 and 26, and SEQ ID NOs: 7, 5, 27 and 17;
SEQ ID NOs: 12, 24, 8 and 10, and SEQ ID NOs: 9, 25, 13 and 11;
SEQ ID NOs: 14, 22, 18, 24 and 20, and SEQ ID NOs: 15, 25, 19, 23 and 21;
SEQ ID NOs: 26, 16, 4 and 6, and SEQ ID NOs: 7, 27, 5 and 17;
SEQ ID NOs: 8, 24, 12 and 10, and SEQ ID NOs: 13, 25, 9 and 11;
SEQ ID NOs: 22, 24, 14, 18 and 20, and SEQ ID NOs: 25, 23, 15, 21 and 19;
SEQ ID NOs: 20, 6, 4, 18, 16 and 26, and SEQ ID NOs: 7, 19, 5, 21, 27 and 17;
SEQ ID NOs: 8, 24, 14, 12, 22 and 10, and SEQ ID NOs: 9, 13, 25, 23, 15 and 11;
SEQ ID NOs: 18, 26, 20, 4, 6 and 16, and SEQ ID NOs: 7, 21, 17, 5, 27 and 19;
SEQ ID NOs: 22, 24, 12, 14, 8 and 10, and SEQ ID NOs: 15, 25, 9, 23, 13 and 11;
SEQ ID NOs: 24, 6, 4, 20, 18 and 32, and SEQ ID NOs: 8, 30, 14, 12, 26 and 10;
SEQ ID NOs: 24, 6, 4, 20, 18 and 32, and SEQ ID NOs: 7, 21, 5, 25, 33 and 19;
SEQ ID NOs: 24, 6, 4, 20, 18 and 32, and SEQ ID NOs: 8, 30, 14, 12, 26 and 10;
SEQ ID NOs: 8, 30, 14, 12, 26 and 10, and SEQ ID NOs: 9, 13, 31, 27, 15 and 11;
SEQ ID NOs: 7, 21, 5, 25, 33 and 19 and SEQ ID NOs: 9, 13, 31, 27, 15 and 11;
SEQ ID NOs: 20, 32, 24, 4, 6 and 18, and SEQ ID NOs: 26, 30, 12, 14, 8 and 10;
SEQ ID NOs: 6, 20, 4, 24, 32, and 18, and SEQ ID NOs: 8, 12, 30, 26, 14 and 10;
SEQ ID NOs: 7, 25, 19, 5, 33 and 21, and SEQ ID NOs: 15, 31, 9, 27, 13 and 11;
SEQ ID NOs: 24, 6, 16 and 18, and SEQ ID NOs: 26, 20, 10 and 28;
SEQ ID NOs: 24, 6, 16 and 18, and SEQ ID NOs: 26, 10, 20 and 28;
SEQ ID NOs: 24, 6, 16 and 18, and SEQ ID NOs: 7, 25, 17 and 19;
SEQ ID NOs: 7, 19, 17 and 25, and SEQ ID NOs: 21, 27, 11 and 29;
SEQ ID NOs: 24, 16, 6 and 18, and SEQ ID NOs: 27, 11, 21 and 29;
SEQ ID NOs: 7, 25, 17 and 19, and SEQ ID NOs: 11, 27, 21 and 29;
SEQ ID NOs: 7, 25, 17 and 19, and SEQ ID NOs: 21, 27, 11 and 29;
SEQ ID NOs: 22, 6, 20 and 28, and SEQ ID NOs: 23, 7, 21 and 29;
SEQ ID NOs: 22, 20, 6 and 28, and SEQ ID NOs: 7, 21, 23 and 29;
SEQ ID NOs: 26, 10, 20 and 28, and SEQ ID NOs: 21, 27, 11 and 29;
SEQ ID NOs: 22, 6, 16, 20, 18, 28, 26 and 10; or.
SEQ ID NOs: 7, 21, 19, 17, 27, 25, 29 and 11.

In some embodiments, the methods entail administering to the subject: one or more fusion polypeptides, or one or more vectors comprising one or more polynucleotides encoding one or more fusion polypeptides, comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the methods entail administering to the subject: one or more fusion polypeptides, or one or more vectors comprising one or more polynucleotides encoding one or more fusion polypeptides, comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223.

In some embodiments, the methods entail administering to the subject: the following first fusion polypeptide and second fusion polypeptide, or one or more vectors comprising one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or.
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively, and (b) a second viral vector comprising one or more polynucleotides encoding the following first fusion polypeptide and second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively, and (b) a second viral vector comprising one or more polynucleotides encoding the following first fusion polypeptide and second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively, and (b) a second viral vector comprising one or more polynucleotides encoding the following first fusion polypeptide and second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively, and (b) a second viral vector comprising one or more polynucleotides encoding the following first fusion polypeptide and second fusion polypeptide comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively, and (b) a second viral vector comprising one or more polynucleotides encoding the following first fusion polypeptide and second fusion polypeptide comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively, and (b) a second viral vector comprising one or more polynucleotides encoding the following first fusion polypeptide and second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and (b) a second viral vector comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and (b) a second viral vector comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively, and (b) a second viral vector comprising one or more polynucleotides encoding the following first fusion polypeptide and second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second viral vector comprising one or more polynucleotides encoding the following first fusion polypeptide and second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and (b) a second viral vector comprising one or more polynucleotides encoding the following first fusion polypeptide and second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively.

In some embodiments, the methods entail administering to the subject first and second viral vectors comprising one or more polynucleotides encoding the following first fusion polypeptide and second fusion polypeptide, optionally joined or connected by one or more linkers: (a) a first viral vector comprising one or more polynucleotides encoding the following first fusion polypeptide and second fusion polypeptide comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively, and (b) a second viral vector comprising one or more polynucleotides encoding the following first fusion polypeptide and second fusion polypeptide comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the methods entail administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 200, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200, and (b) a second polynucleotide or second viral vector or second lipoplex (e.g., LNP) comprising a polynucleotide encoding a second fusion polypeptide comprising SEQ ID NO: 201, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201.

In some embodiments, the methods entail administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 202, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202, and (b) a second polynucleotide or second viral vector or second lipoplex (e.g., LNP) comprising a polynucleotide encoding a second fusion polypeptide comprising SEQ ID NO: 203, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203.

In some embodiments, the methods entail administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 204, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204, and (b) a second polynucleotide or second viral vector or second lipoplex (e.g., LNP) comprising a polynucleotide encoding a second fusion polypeptide comprising SEQ ID NO: 205, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205.

In some embodiments, the methods entail administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 105, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, and (b) a second polynucleotide or second viral vector or second lipoplex (e.g., LNP) comprising a polynucleotide encoding a second fusion polypeptide comprising SEQ ID NO: 107, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107.

In some embodiments, the methods entail administering first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 206, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, and (b) a second polynucleotide or second viral vector or second lipoplex (e.g., LNP) comprising a polynucleotide encoding a second fusion polypeptide comprising SEQ ID NO: 207, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207.

In some embodiments, the methods entail administering g first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 208, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, and (b) a second polynucleotide or second viral vector or second lipoplex (e.g., LNP) comprising a polynucleotide encoding a second fusion polypeptide comprising SEQ ID NO: 209 or SEQ ID NO: 227, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In some embodiments, the methods entail administering g first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 222, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second polynucleotide or second viral vector or second lipoplex (e.g., LNP) comprising a polynucleotide encoding a second fusion polypeptide comprising SEQ ID NO: 209 or SEQ ID NO: 227, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In some embodiments, the methods entail administering g first and second viral vectors or first and second lipoplexes (e.g., LNPs) comprising first and second polynucleotides encoding the following first fusion polypeptide and second fusion polypeptide: (a) a first polynucleotide or first viral vector or first lipoplex (e.g., LNP) comprising a polynucleotide encoding a first fusion polypeptide comprising SEQ ID NO: 222, that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, and (b) a second polynucleotide or second viral vector or second lipoplex (e.g., LNP) comprising a polynucleotide encoding a second fusion polypeptide comprising SEQ ID NO: 223, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223.

In some embodiments, the methods entail administering to the subject a viral vector comprising one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively.

In some embodiments, the methods entail administering to the subject a viral vector comprising one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively.

In some embodiments, the methods entail administering to the subject: the following first fusion polypeptide and second fusion polypeptide, or one or more vectors comprising one or more polynucleotides encoding the following first fusion polypeptide and second fusion polypeptide, in sequential order, from N-terminus to C-terminus, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 77 and 76, or fusion polypeptides that are at least 80%, 81%, 82%, 97%, 98% or 99% identical to any one of SEQ ID NOs: 77 and 76, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 87 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 86, respectively;
SEQ ID NOs: 88 and 89, or fusion polypeptides that are at least 80%, 81%, 82%, 97%, 98% or 99% identical to any one of SEQ ID NOs: 88 and 89, respectively;
SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 87 and 85, or fusion polypeptides that are at least 80%, 81%, 82%, 97%, 98% or 99% identical to any one of SEQ ID NOs: 87 and 85, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 91 and 90, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 91 and 90, respectively;
SEQ ID NOs: 92 and 93, or fusion polypeptides that are at least 80%, 81%, 82%, 97%, 98% or 99% identical to any one of SEQ ID NOs: 92 and 93, respectively;
SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 221 and 220, or fusion polypeptides that are at least 80%, 81%, 82%, 97%, 98% or 99% identical to any one of SEQ ID NOs: 221 and 220, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively;
SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively;
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or
SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively.

In some embodiments, the methods entail administering to the subject: a polynucleotide comprising or consisting of a nucleic acid sequence of any one of SEQ ID NOS: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the methods entail administering to the subject: a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226.

In some embodiments, the methods entail administering to the subject: the following first polynucleotide and second polynucleotide, or one or more vectors comprising or consisting of the following first polynucleotide and second polynucleotide:
SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively;
SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively;
SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively;
SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively;
SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively;
SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively;
SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively;
SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively;
SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively;
SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively;
SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively;
SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively;
SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively;
SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively;
SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively;
SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively;
SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively;
SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively;
SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively;
SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively;
SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively;
SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively;
SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively;
SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively;
SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively;
SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively;
SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively;
SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively;
SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively;
SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively;
SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively;
SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively;
SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively;
SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively;
SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively;
SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or
SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively.

In some embodiments, the method entails administering: (a) a first vector or lipoplex (e.g., LNP) comprising a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and (b) a second vector or lipoplex (e.g., LNP) comprising a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 211.

In some embodiments, the method entails administering: (a) a first vector or lipoplex (e.g., LNP) comprising a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and (b) a second vector or lipoplex (e.g., LNP) comprising a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 213.

In some embodiments, the method entails administering: (a) a first vector or lipoplex (e.g., LNP) comprising a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and (b) a second vector or lipoplex (e.g., LNP) comprising a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 215.

In some embodiments, the method entails administering: (a) a first vector or lipoplex (e.g., LNP) comprising a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and (b) a second vector or lipoplex (e.g., LNP) comprising a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 217.

In some embodiments, the method entails administering: (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 219.

In some embodiments, the method entails administering: (a) a first vector or lipoplex (e.g., LNP) comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 226.

In some embodiments, the method entails administering: (a) a first vector or lipoplex (e.g., LNP) comprising a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and (b) a second vector or lipoplex (e.g., LNP) comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 226.

In some embodiments, the methods entail administering to the subject: a compound fusion polypeptide or a vector comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the methods entail administering to the subject: a compound fusion polypeptide or a vector comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223.

In various embodiments of the methods, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In various embodiments of the methods, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof.

In some embodiments, the methods entail administering one or more viral expression vectors that express one or more of the fusion polypeptides. In various embodiments, the methods entail administering from about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In various embodiments, the methods implement a prime-boost regimen comprising administering a priming composition at a first time point and administering one or more boosting compositions at one or more subsequent time points. Generally, in a prime-boost regimen, the priming composition and the boosting composition are administered sequentially. Illustrative prime-boost regimens include prime-boost-prime-boost and prime-boost-boost-boost. In some embodiments, the administrations of the priming composition and the one or more boosting compositions are spaced at least 1 week, 2 weeks, 3 weeks or 1 month apart, e.g., at least 2, 3, 4, 5, 6, 7, 8, 9, 10, 11 or 12 months apart. In some embodiments, the priming composition and the boosting composition comprise the same immunogenic composition. In some embodiments, the priming composition and the boosting composition comprise different immunogenic compositions. In some embodiments, the priming composition and the boosting composition comprise the same one or more fusion polypeptides and same viral expression vector. In some embodiments, the priming composition and the boosting composition comprise different fusion polypeptides and the same viral expression vectors. In some embodiments, the priming composition and the boosting composition comprise the same fusion polypeptides and different viral expression vectors. In some embodiments, the methods entail priming with a first viral expression vector, and boosting with a second viral expression vector. As appropriate, a prime-boost regimen can be repeated one or more iterations.

In various embodiments, the prime-boost regimen comprises:
Priming with one or more viral expression vectors and boosting with one or more polynucleotides, wherein the one or more polynucleotides comprise DNA, cDNA, mRNA or self-replicating RNA;
Priming with one or more polynucleotides, wherein the one or more polynucleotides comprise DNA, cDNA, RNA, self-amplifying or self-replicating RNA, and boosting with one or more viral expression vectors;
Priming with one or more viral expression vectors, and boosting with one or more viral expression vectors, wherein the one or more viral expression vectors in the priming composition and the one or more viral expression vectors in the boosting composition are from identical, related or unrelated taxonomical families;
Priming with one or more replication-deficient viral expression vectors and boosting with one or more replication-deficient viral expression vectors, wherein the one or more replication-deficient viral expression vectors in the priming composition and the one or more replication-deficient viral expression vectors in the boosting composition are from identical, related or unrelated taxonomical families;
Priming with one or more replication-attenuated viral expression vectors and boosting with one or more replication-attenuated viral expression vectors, wherein the one or more replication-attenuated viral expression vectors in the priming composition and the one or more replication-attenuated viral expression vectors in the boosting composition are from identical, related or unrelated taxonomical families;
Priming with one or more replication-deficient viral expression vectors and boosting with one or more replication-attenuated viral expression vectors;
Priming with one or more replication-attenuated viral expression vectors and boosting with one or more replication-deficient viral expression vectors;
Priming with one or more Lymphocytic choriomeningitis mammarenavirus (LCMV) viral expression vectors and boosting with one or more Pichinde mammarenavirus viral expression vectors;
Priming with one or more Pichinde mammarenavirus viral expression vectors and boosting with one or more Lymphocytic choriomeningitis mammarenavirus (LCMV) viral expression vectors;
Priming with one or more replication deficient Pichinde mammarenavirus viral expression vectors and boosting with one or more replication deficient Lymphocytic choriomeningitis mammarenavirus (LCMV) viral expression vectors;
Priming with one or more replication deficient Lymphocytic choriomeningitis mammarenavirus (LCMV) viral expression vectors and boosting with one or more replication deficient Pichinde mammarenavirus viral expression vectors;
Priming with one or more arenavirus viral expression vectors and boosting with one or more adenovirus viral expression vectors;
Priming with one or more adenovirus viral expression vectors and boosting with boosting composition comprising one or more arenavirus viral expression vectors;
Priming with one or more adenovirus viral expression vectors and boosting with boosting composition comprising one or more RNA molecules (e.g., mRNA, self-amplifying or self-replicating RNA);
Priming with one or more RNA molecules (e.g., mRNA, self-amplifying or self-replicating RNA) and boosting with boosting composition comprising one or more adenovirus viral expression vectors;
Priming with one or more chimpanzee adenoviral (ChAd) expression vectors and boosting with boosting composition comprising one or more self-amplifying or self-replicating RNA (saRNA or samRNA);
Priming with one or more self-amplifying or self-replicating RNA (saRNA or samRNA) and boosting with boosting composition comprising one or more chimpanzee adenoviral (ChAd) expression vectors;
Priming with one or more poxvirus (e.g., Vaccinia) viral expression vectors and boosting with one or more arenavirus viral expression vectors;
Priming with one or more arenavirus viral expression vectors and boosting with boosting composition comprising one or more poxvirus (e.g., Vaccinia) viral expression vectors;
Priming with one or more poxvirus (e.g., Vaccinia) viral expression vectors and boosting with one or more adenovirus viral expression vectors; or
Priming with one or more adenovirus viral expression vectors and boosting with boosting composition comprising one or more poxvirus (e.g., Vaccinia) viral expression vectors.

In some embodiments, the prime-boost regimen comprises:
l) Priming with an immunogenic composition comprising the following first fusion polypeptide and second fusion polypeptide, or one or more vectors comprising one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; and
2) Boosting with an immunogenic composition comprising the following first fusion polypeptide and second fusion polypeptide, or one or more vectors comprising one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively; SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the prime-boost regimen comprises:
l) Priming with an immunogenic composition comprising the following first fusion polypeptide and second fusion polypeptide, or one or more vectors comprising one or more polynucleotides encoding the following first fusion polypeptide and second fusion polypeptide, optionally joined or connected by one or more linkers:
a) SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
b) SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
c) SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
d) SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and/or
e) SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; and
2) Boosting with an immunogenic composition comprising the following first fusion polypeptide and second fusion polypeptide, or one or more vectors comprising one or more polynucleotides encoding the following first fusion polypeptide and second fusion polypeptide, optionally joined or connected by one or more linkers:
a) SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively;
b) SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively;
c) SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and/or
d) SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively; and
   b) a second viral vector comprising one or more polynucleotides encoding first fusion polypeptide and second fusion polypeptides comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively; and/or
   b) a second viral vector comprising one or more polynucleotides encoding first fusion polypeptide and second fusion polypeptides comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and
   b) a second viral vector comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; and/or
   b) a second viral vector comprising one or more polynucleotides encoding first fusion polypeptide and second fusion polypeptides comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and
   b) a second viral vector comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and/or
   b) a second viral vector comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively. In various embodiments of this method, the priming vectors are Cali mammarenavirus (a.k.a., Pichinde mammarenavirus or Pichinde arenavirus) and the boosting vectors are Lymphocytic choriomeningitis mammarenavirus (LCMV). In various embodiments of this method the priming vectors and boosting vectors are replication attenuated or replication competent.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and
   b) a second viral vector comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and/or
   b) a second viral vector comprising one or more polynucleotides encoding first fusion polypeptide and second fusion polypeptides comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively. In various embodiments of this method, the priming vectors are Cali mammarenavirus (a.k.a., Pichinde mammarenavirus or Pichinde arenavirus) and the boosting vectors are Lymphocytic choriomeningitis mammarenavirus (LCMV). In various embodiments of this method the priming vectors and boosting vectors are replication attenuated or replication competent.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and
   b) a second viral vector comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and/or
   b) a second viral vector comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In various embodiments of this method, the priming vectors are Lymphocytic choriomeningitis mammarenavirus (LCMV) and the boosting vectors are Cali mammarenavirus (a.k.a., Pichinde mammarenavirus or Pichinde arenavirus). In various embodiments of this method the priming vectors and boosting vectors are replication attenuated or replication competent.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and
   b) a second viral vector comprising one or more polynucleotides encoding first fusion polypeptide and second fusion polypeptides comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and/or
   b) a second viral vector comprising one or more polynucleotides encoding first fusion polypeptide and second fusion polypeptides comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively. In various embodiments of this method, the priming vectors are Lymphocytic choriomeningitis mammarenavirus (LCMV) and the boosting vectors are Cali mammarenavirus (a.k.a., Pichinde mammarenavirus or Pichinde arenavirus). In various embodiments of this method the priming vectors and boosting vectors are replication attenuated or replication competent.

In some embodiments, the prime-boost regimen comprises:
1) Priming with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, or one or more polynucleotides encoding first fusion polypeptide and second fusion polypeptides comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and
   b) a second viral vector comprising one or more polynucleotides encoding first fusion polypeptide and second fusion polypeptides comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively; and
2) Boosting with an immunogenic composition comprising first and second viral vectors comprising one or more polynucleotides encoding the following first fusion polypeptide and second fusion polypeptide, optionally joined or connected by one or more linkers:
   a) a first viral vector comprising one or more polynucleotides encoding first fusion polypeptide and second fusion polypeptides comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and/or
b) a second viral vector comprising one or more polynucleotides encoding first fusion polypeptide and second fusion polypeptides comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively.

In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the prime-boost regimen comprises: priming and boosting with an immunogenic composition comprising a first viral vector or a lipoplex (e.g., LNP) comprising one or more polynucleotides encoding first fusion polypeptide and second fusion polypeptides, optionally joined or connected by one or more linkers, comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively; and a second viral vector or a lipoplex (e.g., LNP) comprising one or more polynucleotides encoding first fusion polypeptide and second fusion polypeptides, optionally joined or connected by one or more linkers, comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively.

In some embodiments, the prime-boost regimen comprises:
1) Priming with an immunogenic composition comprising a viral vector comprising a polynucleotide encoding a compound fusion polypeptide comprising SEQ ID NO: 105, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105; and
2) Boosting with an immunogenic composition comprising a viral vector comprising a polynucleotide encoding a compound fusion polypeptide comprising SEQ ID NO: 109, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 109.

In some embodiments, the prime-boost regimen comprises:
1) Priming with an immunogenic composition comprising a viral vector comprising a polynucleotide encoding a compound fusion polypeptide comprising SEQ ID NO: 105 or 206, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105 or 206; and
2) Boosting with an immunogenic composition comprising a viral vector comprising a polynucleotide encoding a compound fusion polypeptide comprising SEQ ID NO: 107 or 207, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107 or 207.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 105, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, respectively; and a second viral vector or a lipoplex (e.g., LNP) comprising a polynucleotide encoding a second fusion polypeptide, comprising SEQ ID NO: 107, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107, respectively.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 206, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 206, respectively; and a second viral vector or a lipoplex (e.g., LNP) comprising a polynucleotide encoding a second fusion polypeptide, comprising SEQ ID NO: 207, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 207, respectively.

In some embodiments, the prime-boost regimen comprises:
1) Priming with an immunogenic composition comprising a viral vector comprising a polynucleotide encoding a compound fusion polypeptide comprising SEQ ID NO: 208, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208; and
2) Boosting with an immunogenic composition comprising a viral vector comprising a polynucleotide encoding a compound fusion polypeptide comprising SEQ ID NO: 209 or SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In some embodiments, the prime-boost regimen comprises:
1) Priming with an immunogenic composition comprising a viral vector comprising a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222; and
2) Boosting with an immunogenic composition comprising a viral vector comprising a polynucleotide encoding a compound fusion polypeptide comprising SEQ ID NO: 209 or SEQ ID NO: 227, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227.

In some embodiments, the prime-boost regimen comprises:
1) Priming with an immunogenic composition comprising a viral vector comprising a polynucleotide encoding a compound fusion polypeptide comprising SEQ ID NO: 222, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222; and
2) Boosting with an immunogenic composition comprising a viral vector comprising a polynucleotide encoding a compound fusion polypeptide comprising SEQ ID NO: 223, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 208, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 208, respectively; and a second viral vector or a lipoplex (e.g., LNP) comprising a polynucleotide encoding a second fusion polypeptide, comprising SEQ ID NO: 209 or SEQ ID NO: 227, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In various embodiments of this method, the priming composition comprises one or more ChAd vectors and the boosting composition comprises one or more self-amplifying mRNA molecules, e.g., analogous to the prime-boost regimen described in NCT04776317 and WO 2021/236854 for SARS-COV2 vaccines, in WO 2021/203104 for infectious disease vaccines and in WO 2020/243719.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 222, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and a second viral vector or a lipoplex (e.g., LNP) comprising a polynucleotide encoding a second fusion polypeptide, comprising SEQ ID NO: 209 or SEQ ID NO: 227, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 209 or SEQ ID NO: 227, respectively. In various embodiments of this method, the priming composition comprises one or more ChAd vectors and the boosting composition comprises one or more self-amplifying mRNA molecules, e.g., analogous to the prime-boost regimen described in NCT04776317 and WO 2021/236854 for SARS-COV2 vaccines, in WO 2021/203104 for infectious disease vaccines and in WO 2020/243719.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 222, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 222, respectively; and a second viral vector or a lipoplex (e.g., LNP) comprising a polynucleotide encoding a second fusion polypeptide, comprising SEQ ID NO: 223, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 223, respectively. In various embodiments of this method, the priming composition comprises one or more ChAd vectors and the boosting composition comprises one or more self-amplifying mRNA molecules, e.g., analogous to the prime-boost regimen described in NCT04776317 and WO 2021/236854 for SARS-COV2 vaccines, in WO 2021/203104 for infectious disease vaccines and in WO 2020/243719.

In some embodiments, the prime-boost regimen comprises:
1) Priming with an immunogenic composition comprising a viral vector comprising one or more polynucleotides encoding the following first fusion polypeptide and second fusion polypeptide, optionally joined or connected by one or more linkers: SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively; and
2) Boosting with an immunogenic composition comprising a viral vector comprising one or more polynucleotides encoding the following first fusion polypeptide and second fusion polypeptide, optionally joined or connected by one or more linkers: SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the prime-boost regimen comprises:
1) Priming with an immunogenic composition comprising a viral vector comprising a polynucleotide encoding a compound fusion polypeptide comprising SEQ ID NO: 107, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107; and
2) Boosting with an immunogenic composition comprising a viral vector comprising a polynucleotide encoding a compound fusion polypeptide comprising SEQ ID NO: 111, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 111.

In some embodiments, the prime-boost regimen comprises:
1) Priming with an immunogenic composition comprising a viral vector comprising a polynucleotide encoding a compound fusion polypeptide comprising SEQ ID NO: 200, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 200; and
2) Boosting with an immunogenic composition comprising a viral vector comprising a polynucleotide encoding a compound fusion polypeptide comprising SEQ ID NO: 201, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 201.

In some embodiments, the prime-boost regimen comprises:
1) Priming with an immunogenic composition comprising a viral vector comprising a polynucleotide encoding a compound fusion polypeptide comprising SEQ ID NO: 202, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 202; and
2) Boosting with an immunogenic composition comprising a viral vector comprising a polynucleotide encoding a compound fusion polypeptide comprising SEQ ID NO: 203, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 203.

In some embodiments, the prime-boost regimen comprises:
1) Priming with an immunogenic composition comprising a viral vector comprising a polynucleotide encoding a compound fusion polypeptide comprising SEQ ID NO: 204, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204; and
2) Boosting with an immunogenic composition comprising a viral vector comprising a polynucleotide encoding a compound fusion polypeptide comprising SEQ ID NO: 205, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205.

In some embodiments, the prime-boost regimen comprises: priming and boosting with an immunogenic composition comprising a first viral vector or a lipoplex (e.g., LNP) comprising a polynucleotide encoding a first fusion polypeptide comprising SEQ ID NO: 204, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 204, respectively; and a second viral vector or a lipoplex (e.g., LNP) comprising a polynucleotide encoding a second fusion polypeptide, comprising SEQ ID NO: 205, or a fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 205, respectively.

In some embodiments, the prime-boost regimen comprises:
1) Priming with an immunogenic composition comprising first and second viral vectors comprising the following polynucleotides:
   a) a first viral vector comprising first and second polynucleotides comprising SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; and
   b) a second viral vector comprising first and second polynucleotides comprising SEQ ID NOs: 133 and 137, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively; and
2) Boosting with an immunogenic composition comprising first and second viral vectors comprising the following polynucleotides:
   a) a first viral vector comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and
   b) a second viral vector comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively.

In some embodiments, the prime-boost regimen comprises:
1) Priming with an immunogenic composition comprising first and second viral vectors comprising the following polynucleotides:
a) a first viral vector comprising first and second polynucleotides comprising SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and
b) a second viral vector comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively; and
2) Boosting with an immunogenic composition comprising first and second viral vectors comprising the following polynucleotides:
   a) a first viral vector comprising first and second polynucleotides comprising SEQ ID NOs: 131 and 135, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively; and
   b) a second viral vector comprising first and second polynucleotides comprising SEQ ID NOs: 133 and 137, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively.

In some embodiments, the prime-boost regimen comprises:
1) Priming with an immunogenic composition comprising first and second viral vectors comprising the following polynucleotides:
   a) a first viral vector comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; and
   b) a second viral vector comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively; and
2) Boosting with an immunogenic composition comprising first and second viral vectors comprising the following polynucleotides:
   a) a first viral vector comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and
   b) a second viral vector comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In various embodiments of this method, the priming vectors are Cali mammarenavirus (a.k.a., Pichinde mammarenavirus or Pichinde arenavirus) and the boosting vectors are Lymphocytic choriomeningitis mammarenavirus (LCMV). In various embodiments of this method the priming vectors and boosting vectors are replication attenuated or replication competent.

In some embodiments, the prime-boost regimen comprises:
1) Priming with an immunogenic composition comprising first and second viral vectors comprising the following polynucleotides:
   a) a first viral vector comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and
   b) a second viral vector comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively; and
2) Boosting with an immunogenic composition comprising first and second viral vectors comprising the following polynucleotides:
   a) a first viral vector comprising first and second polynucleotides comprising SEQ ID NOs: 140 and 146, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively; and
   b) a second viral vector comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively. In various embodiments of this method, the priming vectors are Cali mammarenavirus (a.k.a., Pichinde mammarenavirus or Pichinde arenavirus) and the boosting vectors are Lymphocytic choriomeningitis mammarenavirus (LCMV). In various embodiments of this method the priming vectors and boosting vectors are replication attenuated or replication competent.

In some embodiments, the prime-boost regimen comprises:
1) Priming with an immunogenic composition comprising first and second viral vectors comprising the following polynucleotides:
   a) a first viral vector comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and
   b) a second viral vector comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; and
2) Boosting with an immunogenic composition comprising first and second viral vectors comprising the following polynucleotides:
   a) a first viral vector comprising first and second polynucleotides comprising SEQ ID NOs: 151 and 153, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; and
   b) a second viral vector comprising first and second polynucleotides comprising SEQ ID NOs: 156 and 159, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively.

In some embodiments, the prime-boost regimen comprises:
1) Priming with an immunogenic composition comprising first and second viral vectors comprising the following polynucleotides:
   a) a first viral vector comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and
   b) a second viral vector comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively; and
2) Boosting with an immunogenic composition comprising first and second viral vectors comprising the following polynucleotides:
   a) a first viral vector comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and
   b) a second viral vector comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In various embodiments of this method, the priming vectors are Cali mammarenavirus (a.k.a., Pichinde mammarenavirus or Pichinde arenavirus) and the boosting vectors are Lymphocytic choriomeningitis mammarenavirus (LCMV). In various embodiments of this method the priming vectors and boosting vectors are replication attenuated or replication competent.

In some embodiments, the prime-boost regimen comprises:
1) Priming with an immunogenic composition comprising a first viral vector comprising first and second polynucleotides comprising SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively; and
2) Boosting with an immunogenic composition comprising a second viral vector comprising first and second polynucleotides comprising SEQ ID NOs: 162 and 163, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively.

In some embodiments, the prime-boost regimen comprises:
1) Priming with an immunogenic composition comprising a first viral vector comprising first and second polynucleotides comprising SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively; and
2) Boosting with an immunogenic composition comprising a second viral vector comprising first and second polynucleotides comprising SEQ ID NOs: 166 and 167, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively.

In some embodiments, the prime-boost regimen comprises: priming and boosting with an immunogenic composition comprising a first viral vector comprising a first polynucleotide comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210; and a second viral vector comprising a second polynucleotide comprising SEQ ID NO: 211, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211.

In some embodiments, the prime-boost regimen comprises: priming and boosting with an immunogenic composition comprising a first viral vector comprising a first polynucleotide comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212; and a second viral vector comprising a second polynucleotide comprising SEQ ID NO: 213, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213.

In some embodiments, the prime-boost regimen comprises: priming and boosting with an immunogenic composition comprising a first viral vector comprising a first polynucleotide comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214; and a second viral vector comprising a second polynucleotide comprising SEQ ID NO: 215, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215.

In some embodiments, the prime-boost regimen comprises: priming and boosting with an immunogenic composition comprising a first viral vector comprising a first polynucleotide comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216; and a second viral vector comprising a second polynucleotide comprising SEQ ID NO: 217, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217.

In some embodiments, the prime-boost regimen comprises: priming and boosting with an immunogenic composition comprising a first viral vector comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218; and a second viral vector comprising a second polynucleotide comprising SEQ ID NO: 219, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219.

In some embodiments, the prime-boost regimen comprises: priming and boosting with an immunogenic composition comprising a first viral vector comprising a first polynucleotide comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218; and a second viral vector comprising a second polynucleotide comprising SEQ ID NO: 226, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In various embodiments of this method, the priming composition comprises one or more ChAd vectors and the boosting composition comprises one or more self-amplifying mRNA molecules, e.g., analogous to the prime-boost regimen described in NCT04776317 and WO 2021/236854 for SARS-COV2 vaccines, in WO 2021/203104 for infectious disease vaccines and in WO 2020/243719.

In some embodiments, the prime-boost regimen comprises: priming and boosting with an immunogenic composition comprising a first viral vector comprising a first polynucleotide comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225; and a second viral vector comprising a second polynucleotide comprising SEQ ID NO: 226, or that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226. In various embodiments of this method, the priming composition comprises one or more ChAd vectors and the boosting composition comprises one or more self-amplifying mRNA molecules, e.g., analogous to the prime-boost regimen described in NCT04776317 and WO 2021/236854 for SARS-COV2 vaccines, in WO 2021/203104 for infectious disease vaccines and in WO 2020/243719.

In some embodiments, after one or more administrations of the one or more fusion polypeptides, as described herein, or one or more polynucleotides encoding one or more fusion polypeptides, as described herein, or one or more vectors expressing one or more fusion polypeptides, as described herein, optionally with one or more additional therapeutic agents, described herein, the subject does not exhibit symptoms of HIV or AIDS in the absence of anti-retroviral treatment (ART) for at least 3 months, at least 6 months, at least 1 year, at least 2 years, at least 3 years, or more. In some embodiments, after one or more administrations of the one or more fusion polypeptides, as described herein, or one or more polynucleotides encoding one or more fusion polypeptides, as described herein, or one or more vectors expressing one or more fusion polypeptides, as described herein, optionally with one or more additional therapeutic agents, the subject has a viral load of copies/ml blood of less than 500, e.g., less than 400, less than 300, less than 200, less than 100, less than 50, in the absence of anti-retroviral treatment (ART) for at least 3 months, at least 6 months, at least 1 year, at least 2 years, at least 3 years, or more.

### 7. Combination Therapies

In certain embodiments, a method for treating or preventing an HIV infection in a human having or at risk of having the infection is provided, comprising administering to the human a therapeutically effective amount of one or more fusion polypeptides, or polynucleotides encoding or vectors expressing such fusion polypeptides, as disclosed herein, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of a compound disclosed herein, or a pharmaceutically acceptable salt thereof, in combination with a therapeutically effective amount of one or more (e.g., one, two, three, one or two, or one to three) additional therapeutic agents.

In various embodiments, of one or more fusion polypeptides, or polynucleotides encoding or vectors expressing such fusion polypeptides, as disclosed herein, are administered in combination with one or more (e.g., one, two, three, four, one or two, one to three, or one to four) additional therapeutic agents, and a pharmaceutically acceptable carrier, diluent, or excipient.

In certain embodiments, the provided are methods for treating an HIV infection, comprising administering to a patient in need thereof a therapeutically effective amount of a compound disclosed herein, or a pharmaceutically acceptable salt thereof, in combination with a therapeutically effective amount of one or more (e.g., one, two, three, one or two, or one to three) additional therapeutic agents which are suitable for treating an HIV infection.

In certain embodiments, one or more fusion polypeptides, or polynucleotides encoding or vectors expressing such fusion polypeptides, as disclosed herein, is co-formulated with one, two, three, four, or more additional therapeutic agents, and a pharmaceutically acceptable carrier. In certain embodiments, one or more fusion polypeptides, or polynucleotides encoding or vectors expressing such fusion polypeptides, as disclosed herein, or a pharmaceutically acceptable salt thereof, is combined with two additional therapeutic agents. As appropriate, the one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a one or more fusion polypeptides, or polynucleotides encoding or vectors expressing such fusion polypeptides, as disclosed herein, are administered with one or more additional therapeutic agents. Co-administration of a compound disclosed herein with one or more additional therapeutic agents generally refers to simultaneous or concurrent, or sequential, administration of a compound disclosed herein and one or more additional therapeutic agents, such that therapeutically effective amounts of the compound disclosed herein and the one or more additional therapeutic agents are both present in the body of the patient. When administered sequentially, the combination may be administered in two or more administrations.

Co-administration includes administration of unit dosages of a unit dose of the one or more fusion polypeptides, or polynucleotides encoding, lipoplexes (e.g., LNPs) comprising such polynucleotides, or vectors expressing such fusion polypeptides, as disclosed herein, disclosed herein before or after administration of unit dosages of one or more additional therapeutic agents. For example, the one or more fusion polypeptides, or polynucleotides encoding, lipoplexes (e.g., LNPs) comprising such polynucleotides, or vectors expressing such fusion polypeptides, as disclosed herein, may be administered within seconds, minutes, or hours of the administration of the one or more additional therapeutic agents. In some embodiments, a unit dose of a one or more fusion polypeptides, or polynucleotides encoding or vectors expressing such fusion polypeptides, as disclosed herein, is administered first, followed within seconds or minutes by administration of a unit dose of one or more additional therapeutic agents.

Alternatively, a unit dose of one or more additional therapeutic agents is administered first, followed by administration of a unit dose of a one or more fusion polypeptides, or polynucleotides encoding or vectors expressing such fusion polypeptides, as disclosed herein, within seconds or minutes. In other embodiments, a unit dose of one or more fusion polypeptides, or polynucleotides encoding, lipoplexes (e.g., LNPs) comprising such polynucleotides, or vectors expressing such fusion polypeptides, as disclosed herein, is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of one or more fusion polypeptides, or polynucleotides encoding or vectors expressing such fusion polypeptides, as disclosed herein. In some embodiments, the one or more additional therapeutic agents are not co-administered, but are separately administered as part of a combination therapy regimen. In such approaches, a unit dose of one or more fusion polypeptides, or polynucleotides encoding, lipoplexes (e.g., LNPs) comprising such polynucleotides, or vectors expressing such fusion polypeptides, as disclosed herein, and the one or more additional therapeutic agents can be administered at longer time intervals, e.g., 1, 2, 3, 4, 5, 6, 7, 8, 9, 10 days or more apart.

In certain embodiments, one or more fusion polypeptides, or polynucleotides encoding or vectors expressing such fusion polypeptides, as disclosed herein, is combined or co-administered with one or more additional therapeutic agents in a unitary dosage form for simultaneous or concurrent administration to a patient, for example as an aqueous formulation for intravenous, intramuscular, intradermal, intranodal or subcutaneous administration. In certain embodiments, one or more fusion polypeptides, or polynucleotides encoding or vectors expressing such fusion polypeptides, as disclosed herein, is combined with one or more additional therapeutic agents in a unitary dosage form for simultaneous or concurrent administration to a patient, for example as an intrarectal suppository.

In certain embodiments, the one or more fusion polypeptides, or polynucleotides encoding, lipoplexes (e.g., LNPs) comprising such polynucleotides, or vectors expressing such fusion polypeptides, as disclosed herein, can be co-formulated or co-administered with one or more other compounds useful for treating HIV. In certain embodiments, the co-formulation or co-administration can comprise another active agent for treating HIV, such as anti-HIV antibodies (e.g., HIV bNAbs), bispecific antibodies, and “antibody-like” therapeutic proteins, toll-like receptor (TLR) agonists (e.g., agonists of TLR7, TLR8, and/or TLR9), an immune checkpoint inhibitor, HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, nucleocapsid protein 7 (NCp7) inhibitors, HIV Tat or Rev inhibitors, inhibitors of Tat-TAR-P-TEFb, HIV non-catalytic site (or allosteric) integrase inhibitors, HIV entry (fusion) inhibitors, HIV maturation inhibitors, latency reversing agents, immune-based therapies, PI3K inhibitors, gp41 inhibitors, CXCR4 inhibitors, gp120 inhibitors, CCR5 inhibitors, Nef inhibitors, latency reversing agents, HIV vaccines, cytokines, immune checkpoint inhibitors, FLT3 agonists, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are selected from dolutegravir, cabotegravir, islatravir, darunavir, bictegravir, elsulfavirine, rilpivirine, and lenacapavir, and combinations thereof.

In certain embodiments, the one or more active agents are suitable for once daily dosing, weekly dosing, monthly dosing, every 3 months dosing, every four months dosing, bi-annual dosing, or annual dosing, as appropriate.

In some embodiments, the one or more fusion polypeptides, or polynucleotides encoding, lipoplexes (e.g., LNPs) comprising such polynucleotides, or vectors expressing such fusion polypeptides, as disclosed herein, and the one or more additional therapeutic agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, interferon (IFN) antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, prolylendopeptidase inhibitors, phospholipase A2 inhibitors, pharmacokinetic enhancers, HIV gene therapy, HIV vaccines, anti-HIV peptides and combinations thereof.

In some embodiments, the additional therapeutic agent is selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

### Combination Drugs

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV combination drug. Examples of combination drugs that can be employed with an agent of this disclosure include ATRIPLA® (efavirenz, tenofovir disoproxil fumarate, and emtricitabine); COMPLERAR (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXAR; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir +lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

Examples of other drugs for treating HIV that can be combined with the one or more fusion polypeptides, or polynucleotides encoding, lipoplexes (e.g., LNPs) comprising such polynucleotides, or vectors expressing such fusion polypeptides, as disclosed herein, include without limitation aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BG-HIV, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591, islatravir, NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV protease inhibitor. Examples of HIV protease inhibitors that can be co-administered or combined include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234 and U.S. Patent Publ. Nos. US2020030327 and US2019210978.

### HIV Gag Protein Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV Gag protein inhibitor. Examples of HIV Gag protein inhibitors that can be combined or co-administered include, but are not limited to, HRF-10071.

### HIV Ribonuclease H Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV ribonuclease H inhibitor. Examples of HIV ribonuclease H inhibitors that can be combined or co-administered include, but are not limited to, NSC-727447.

### HIV Nef Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV Nef inhibitor. Examples of HIV Nef inhibitors that can be combined or co-administered include, but are not limited to, FP-1.

### HIV Reverse Transcriptase Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a non-nucleoside or non-nucleotide inhibitor. Examples of HIV non-nucleoside or non-nucleotide inhibitors of reverse transcriptase that can be combined or co-administered include dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV nucleoside or nucleotide inhibitor. Examples of HIV nucleoside or nucleotide inhibitors of reverse transcriptase that can be combined or co-administered include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500 and KP-1461.

### HIV Integrase Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV integrase inhibitor. Examples of HIV integrase inhibitors that can be combined or co-administered include without limitation elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, ACC-017 and cabotegravir.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a HIV non-catalytic site, or allosteric, integrase inhibitor (NCINI). Examples of HIV non-catalytic site, or allosteric, integrase inhibitors (NCINI) that can be combined or co-administered include CX-05045, CX-05168, and CX-14442. Additional examples of HIV capsid inhibitors include, without limitation those described in U.S. Patent Publ. Nos. US2014221356 and US2016016973.

### HIV Viral Infectivity Factor Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a HIV Viral Infectivity Factor Inhibitor. Examples of HIV viral infectivity factor inhibitors include, but are not limited to 2-amino-N-(2-methoxyphenyl)-6-((4-nitrophenyl) thio) benzamide derivatives and Irino-L.

### HIV Entry Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV entry inhibitor. Examples of HIV entry (fusion) inhibitors that can be combined or co-administered include without limitation AAR-501, LBT-5001, cenicriviroc, CCR5 inhibitors, gp41 inhibitors, CD4 attachment inhibitors, gp120 inhibitors, gp160 inhibitors, and CXCR4 inhibitors.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a CCR5 inhibitor. Examples of CCR5 inhibitors that can be combined or co-administered include without limitation aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a gp41 inhibitor. Examples of gp41 inhibitors that can be combined or co-administered include without limitation albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a CD4 attachment inhibitor. Examples of CD4 attachment inhibitors that can be combined or co-administered include ibalizumab and CADA analogs.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a gp120 inhibitor. Examples of gp120 inhibitors that can be combined or co-administered include without limitation anti-HIV microbicide, Radha-108 (receptol) 3B3-PE38, BMS818251, BanLec, bentonite-based nanomedicine, fostemsavir tromethamine, IQP-0831, VVX-004, and BMS-663068.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a gp160 inhibitor. Examples of gp160 inhibitors that can be combined or co-administered include without limitation fangchinoline.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a CXCR4 inhibitor. Examples of CXCR4 inhibitors that can be combined or co-administered include plerixafor, ALT-1188, N15 peptide, and vMIP (Haimipu).

### Maturation Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a HIV maturation inhibitor. Examples of HIV maturation inhibitors that can be combined or co-administered include BMS-955176, GSK-3640254 and GSK-2838232.

### Latency Reversing Agents

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a latency reversing agent (LRA). Examples of latency reversing agents that can be combined or co-administered include toll-like receptor (TLR) agonists (including TLR7 agonists, e.g., GS-9620 (vesatolimod), TLR8 agonists, e.g., GS-9688 (Selgantolimod), TLR9 agonists, e.g., lefitolimod (MGN-1703)), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to indolactam, prostratin, ingenol B, and DAG-lactones. Additional examples of TLR7 agonists includes but are not limited to those described in U.S. Patent No. US2010/143301. Additional examples of TLR8 agonists includes but are not limited to those described in U.S. Patent No. US2017/071944.

### Histone Deacetylase (HDAC) Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an inhibitor of a histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a capsid inhibitor. Examples of capsid inhibitors that can be combined or co-administered include without limitation capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (WO 2019/087016 (GSK)). Additional examples of capsid inhibitors include without limitation those described in US Patent Publ. Nos. US2018051005, US2016108030.

### HIV Long-Acting Therapy

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an HIV long-acting therapy. Examples of as long acting regimens that can be combined or co-administered include without limitation cabotegravir, rilpivirine, any integrase LA, VM-1500 LAI, maraviroc (LAI), tenofovir implant, islatravir implant, doravirine, raltegravir, and long acting dolutegravir.

Additionally encompassed as HIV long-acting therapies include anti-HIV broadly neutralizing antibodies (bNAbs), described herein, having serum half-life extended amino acid substitutions in the Fc region. Fc region amino acid substitutions that increase the half-life of an antibody have been described. For example, a “YTE mutant” (a methionine to tyrosine substitution at position 252, a serine to threonine substitution at position 254, and a threonine to glutamic acid substitution at position 256 (EU numbering)) exhibits a four-fold increased half-life relative to wild-type versions of the same antibody (Dall'Acqua, et al., J Biol Chem, 281:23514-24 (2006); Robbie, et al., Antimicrob Agents Chemotherap., 57 (12): 6147-6153 (2013)). See also, U.S. Pat. No. 7,658,921. In a further example, M428L and N434S (EU numbering; “LS”) substitutions can increase the pharmacokinetic half-life of the bNAb. In other embodiments, the bNAbs described herein comprise T250Q and M428L (EU numbering) mutations. In other embodiments, the bNAbs described herein comprise H433K and N434F (EU numbering) mutations. Illustrative serum half-life extended bNAbs that may be combined or co-administered with the one or more fusion polypeptides, or polynucleotides encoding, lipoplexes (e.g., LNPs) comprising such polynucleotides, or vectors expressing such fusion polypeptides, as disclosed herein, include 3BNC117-LS, 10-1074-LS, 10-1074-LS-J, GS-5423 and GS-2872.

### Cytochrome P450 3 Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a Cytochrome P450 3 inhibitor. Examples of Cytochrome P450 3 inhibitors include without limitation those described in U.S. Pat. No. 7,939,553.

### RNA Polymerase Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an RNA polymerase modulator. Examples of RNA polymerase modulators include without limitation those described in U.S. Pat. Nos. 10,065,958; and 8,008,264.

### Immune Checkpoint Modulators

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18 (11): 671-688).

Examples of immune checkpoint proteins or receptors include without limitation CD27 (NCBI Gene ID: 939), CD70 (NCBI Gene ID: 970), CD40 (NCBI Gene ID: 958), CD40LG (NCBI Gene ID: 959), CD47 (NCBI Gene ID: 961), CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4; NCBI Gene ID: 79679); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; CD279; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL 16 binding protein 1 (ULBP1; NCBI Gene ID: 80329); UL16 binding protein 2 (ULBP2; NCBI Gene ID: 80328); UL 16 binding protein 3 (ULBP3; NCBI Gene ID: 79465); retinoic acid early transcript 1E (RAETIE; ULBP4; NCBI Gene ID: 135250); retinoic acid early transcript 1G (RAETIG; ULBP5; NCBI Gene ID: 353091); retinoic acid early transcript 1L (RAETIL; ULBP6; NCBI Gene ID: 154064); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1, KIR, CD158E1; NCBI Gene ID: 3811) (e.g., Lirilumab (IPH2102/BMS-986015), IPH-4102); killer cell lectin like receptor D1 (KLRD1; NCBI Gene ID: 3824) and mitogen-activated protein kinase kinase kinase kinase 1 (MAP4K1, a.k.a., Hematopoietic Progenitor Kinase 1 (HPK1); NCBI Gene ID: 11184).

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In various embodiments, the fusion polypeptides, polynucleotides, vectors, LNPs, immunogenic compositions and/or pharmaceutical compositions, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCDI is selected from GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with anti-TIGIT antibodies, such as etigilimab, BMS-986207, tiragolumab (a.k.a., MTIG-7192A; RG-6058; RO 7092284), vibostolimab (MK-7684), AGEN1307, AGEN1327, AGEN1777, COM-902, IBI-939, AB154, SGN-TGT, MG1131 and EOS884448 (EOS-448).

### TNF Receptor Superfamily (TNFRSF) Member Agonists or Activators

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with one or more agonists of one or more TNF receptor superfamily (TNFRSF) members, e.g., an agonist of one or more of TNFRSFIA (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF1OD (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (4-1BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRIKE) include OXS-3550, HIV-TriKE and CD16-IL-15-B7H3 Trike.

### Indoleamine-Pyrrole-2,3-Dioxygenase (IDO1) Inhibitors

In various embodiments, the one or more fusion polypeptides, or polynucleotides encoding, lipoplexes (e.g., LNPs) comprising such polynucleotides, or vectors expressing such fusion polypeptides, as disclosed herein, are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Toll-Like Receptor (TLR) Agonists

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262, telratolimod and BDB-001. Example TLR8 agonists that can be co-administered include without limitation E-6887, IMO-4200, IMO-8400, IMO-9200, MCT-465, MEDI-9197, motolimod, resiquimod, GS-9688 (Selgantolimod), VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008 (cavrotolimod), cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonists include rintatolimod, poly-ICLC, RIBOXXON®, Apoxxim, RIBOXXIM®, IPH-33, MCT-465, MCT-475, and ND-1.1. Examples of TLR4 agonists include G-100, and GSK-1795091.

### CDK Inhibitors or Antagonists

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a CDK inhibitor or antagonist. In some embodiments, the agents described herein are combined with an inhibitor or antagonist of CDK. In some embodiments, the CDK inhibitor or antagonist is selected from VS2-370.

### STING Agonists, RIG-I and NOD2 Modulators

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a stimulator of interferon genes (STING) receptor (a.k.a, stimulator of interferon response cGAMP interactor 1 (STING1); transmembrane protein 173 (TMEM173); NCBI Gene ID: 340061) agonist. In some embodiments, the STING receptor agonist or activator is selected from ADU-S100 (MIW-815), SB-11285, MK-1454, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

In some embodiments, the additional therapeutic agent is an agonist of DExD/H-box helicase 58 (DDX58; a.k.a., RIG-I, RIGI, RIGI, RLR-1, SGMRT2; NCBI Gene ID: 23586). Illustrative RIG-I agonists include inarigivir soproxil (SB-9200; GS-9992); SB-40, SB-44, ORI-7246, ORI-9350, ORI-7537, ORI-9020, ORI-9198, ORI-7170, RGT-100 and KIN1148, described by Hemann, et al., J Immunol May 1, 2016, 196 (1 Supplement) 76.1. Additional RIG-I agonists are described, e.g., in Elion, et al., Cancer Res. (2018) 78 (21): 6183-6195; and Liu, et al., J Virol. (2016) 90 (20): 9406-19. RIG-I agonists are commercially available, e.g., from Invivogen (invivogen.com). In some embodiments, the agents described herein are combined with a nucleotide binding oligomerization domain containing 2 (NOD2; NCBI Gene ID: 64127) agonist, such as inarigivir soproxil (SB-9200; GS-9992) and IR-103.

LAG-3 and TIM-3 inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an anti-TIM-3 antibody, such as TSR-022, LY-3321367, MBG-453, INCAGN-2390.

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with anti-LAG-3 (Lymphocyte-activation) antibody, such as relatlimab (ONO-4482), LAG-525, MK-4280, REGN-3767, INCAGN2385.

### Interleukin or Cytokine Receptor Agonists

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an interleukin receptor agonist, such as IL-2, IL-7, IL-15, IL-10, IL-12 receptor agonists. Examples of IL-2 receptor agonists that can be co-administered include proleukin (aldesleukin, IL-2); BC-IL (Cel-Sci), pegylated IL-2 (e.g., NKTR-214); modified variants of IL-2 (e.g., THOR-707, Fc-IL-2 fusion protein), bempegaldesleukin, AIC-284, ALKS-4230, CUI-101, Neo-2/15. Examples of IL-15 receptor agonists that can be co-administered include ALT-803, NKTR-255, and hetIL-15, interleukin-15/Fc fusion protein, AM-0015, NIZ-985, SO-C101, IL-15 Synthorin (pegylated Il-15), P-22339, and an IL-15-PD-1 fusion protein N-809. Examples of IL-7 receptor agonist that can be co-administered include CYT-107.

Examples of additional immune-based therapies that can be combined or co-administered include interferon alfa; interferon alfa-2b; interferon alfa-n3; pegylated interferon alfa; interferon gamma; fms related tyrosine kinase 3 (FLT3) agonists (e.g., GS-3583, CDX-301); gepon; normferon, peginterferon alfa-2a, peginterferon alfa-2b, RPI-MN. In various embodiments, a fms related tyrosine kinase 3 (FLT3) agonists (e.g., GS-3583, CDX-301) is administered at a first time point and the one or more fusion polypeptides, or polynucleotides encoding, lipoplexes (e.g., LNPs) comprising such polynucleotides, or vectors expressing such fusion polypeptides are administered at a time point at least 6, 7, 8, 9, 10 days, e.g., 1 week, after the first time point.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an fms related tyrosine kinase 3 (FLT3) agonist (e.g., GS-3583, CDX-301). Illustrative FLT3 agonists that can be co-administered are described, e.g., in WO 2020/263830A1. In various embodiments, the co-administration of a FLT3 agonist increases the vaccine-induced T cell response in comparison to administration of the one or more fusion polypeptides, or polynucleotides encoding, lipoplexes (e.g., LNPs) comprising such polynucleotides, or vectors expressing such fusion polypeptides in the absence of a FLT3 agonist.

Phosphatidylinositol 3-kinase (PI3K) Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a PI3K inhibitor. Examples of PI3K inhibitors that can be combined or co-administered include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Diacylglycerol Kinase Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an inhibitor of a diacylglycerol kinase (DGK), e.g., diacylglycerol kinase alpha (DGKa) and diacylglycerol kinase zeta (DGKč). Examples of DGK inhibitors that can be combined or co-administered include ritanserin and the DGK inhibitors described in WO2020006016 and WO2020006018.

alpha-4/beta-7 antagonists

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an alpha-4/beta-7 antagonist. Examples of Integrin alpha-4/beta-7 antagonists that can be combined or co-administered include PTG-100, TRK-170, abrilumab, etrolizumab, carotegrast methyl, and vedolizumab.

### HPK1/MAP4K1 Inhibitors

In certain embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with an inhibitor of mitogen-activated protein kinase kinase kinase kinase 1 (MAP4K1, a.k.a., Hematopoietic Progenitor Kinase 1 (HPK1); NCBI Gene ID: 11184). Examples of HPK1 inhibitors include, but are not limited to, ZYF-0272, and ZYF-0057.

### HIV-Targeting Antibodies

Examples of HIV antibodies, bispecific antibodies, and “antibody-like” therapeutic proteins that can be combined or co-administered include DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs, ibalizumab, ibalizumab (second generation), Immuglo, MB-66, clone 3 human monoclonal antibody targeting KLIC (HIV infection). Various bNAbs may be used, as described herein.

In certain embodiments, the co-administered antibody or antigen-binding fragment thereof, or an antigen-binding molecule, is or is derived from human neutralizing antibodies (e.g., monoclonal) that target HIV-1. A “neutralizing antibody” is one that can neutralize the ability of HIV to initiate and/or perpetuate an infection in a host and/or in target cells in vitro. The disclosure provides neutralizing monoclonal human antibodies, wherein the antibody recognizes an antigen from HIV, e.g., a gp120 polypeptide. In certain embodiments, a “neutralizing antibody” may inhibit the entry of HIV-1 virus, e.g., SF162 and/or JR-CSF, with a neutralization index>1.5 or >2.0 (Kostrikis L G et al., J. Virol., 70 (1): 445-458 (1996)).

In some embodiments, the co-administered antibody or antigen-binding fragment thereof, or an antigen-binding molecule, is or is derived from a human broadly neutralizing antibody (e.g., monoclonal) that target HIV-1. By “broadly neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. Illustrative broadly neutralizing antibodies (bNAbs) which can be co-administered as an additional therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Illustrative bNAbs that can be co-administered include without limitation 12A12, 12A21, NIH45-46, bANC131, 8ANC134, IB2530, INC9, 8ANC195, 8ANC196, 10-259, 10-303, 10-410, 10-847, 10-996, 10-1074, 10-1074-LS, 10-1074-LS-J, 10-1121, 10-1130, 10-1146, 10-1341, 10-1369, 10-1074GM, PGT-121, PGT-121.414, GS-9721, GS-9722, GS-2872. Additional examples include those described in Sajadi, et al., Cell. (2018) 173 (7): 1783-1795; Sajadi, et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid, et al., Science, 333:1633-1637 (2011), Scheid, et al., Nature, 458:636-640 (2009), Eroshkin et al, Nucleic Acids Res., 42 (Database issue): D1 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind VIV2-glycan), 2G12 (which binds to outer domain glycan); b12, HJ16, CH130-149, VRC01-03, VRC-PG04, 04b, VRC-CH30-34, 3BNC62, 3BNC89, 3BNC91, 3BNC95, 3BNC104, 3BNC117, 3BNC176, 8ANC131, GS-9723, GS-5423 (all of which bind to the CD4 binding site), which are hereby incorporated herein by reference in their entireties for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp 120 selected from: (i) the third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120. The foregoing epitopes or regions of gp 120 bound by broadly neutralizing antibodies are described, e.g., in McCoy, Retrovirology (2018) 15:70; Sok and Burton, Nat Immunol. 2018 19 (11): 1179-1188; Possas, et al., Expert Opin Ther Pat. 2018 July; 28 (7): 551-560; and Stephenson and Barouch, Curr HIV AIDS Rep (2016) 13:31-37, which are hereby incorporated herein by reference in their entirety for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb) that binds to an epitope or region of gp 120 in the third variable loop (V3) and/or high mannose patch comprising a N332 oligomannose glycan and competes with or comprises VH and VL regions from an antibody selected from GS-9722, PGT-121.60,

PGT-121.66, PGT-121, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp 120 in the third variable loop (V3) and/or high mannose patch comprising a N332 oligomannose glycan and which can be used as the second antibody or antigen-binding fragment thereof are described, e.g., in WO 2012/030904; WO 2014/063059; WO 2016/149698; WO 2017/106346; WO 2018/075564, WO 2018/125813 and WO 2018/237148, which are hereby incorporated herein by reference in their entireties for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp 120 in the CD4 binding site (CD4bs) and competes with or comprises CDRs and/or VH and VL regions from an antibody selected from b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N49-P7, NC-Cow1, IOMA, CH235 and CH235.12, N49P6, N49P7, N49P11, N49P9 and N60P25. Additional broadly neutralizing antibodies that bind to gp120 CD4 binding site (CD4bs) and which can be used as the second antibody or antigen-binding fragment thereof are described, e.g., in WO 2012/154312, WO 2012/158948, WO 2013/090644, WO 2013/192589, WO 2018/125813, which are hereby incorporated herein by reference in their entireties for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp 120 in the second variable loop (V2) and/or Env trimer apex and competes with or comprises VH and VL regions from an antibody selected from PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. Additional broadly neutralizing antibodies that bind to gp120 in the second variable loop (V2) and/or Env trimer apex and which can be used as the second antibody or antigen-binding fragment thereof are described, e.g., in WO 2010/107939; WO 2012/030904; WO 2018/075564 and WO 2018/125813, which are hereby incorporated herein by reference in their entireties for all purposes.

In some embodiments the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp 120 in the gp120/gp41 interface and competes with or comprises VH and VL regions from an antibody selected from PGT-151, CAP248-2B, 35022, 8ANC195, ACS202, VRC34 and VRC34.01. Additional broadly neutralizing antibodies that bind to gp 120 in the gp120/gp41 interface and which can be used as the second antibody or antigen-binding fragment thereof are described, e.g., in WO 2011/038290; WO 2012/030904 and WO2017/079479, which are hereby incorporated herein by reference in their entireties for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, combined or co-administered with a broadly neutralizing antibody (bNAb)) that binds to an epitope or region of the gp 120 silent face and competes with or comprises VH and VL regions from an antibody selected from VRC-PG05 and SF12. See, e.g., Schoofs, et al., “Broad and Potent Neutralizing Antibodies Recognize the Silent Face of the HIV Envelope,” Immunity (2019) May 14. pii: S1074-7613 (19) 30194-3 (PMID 31126879).

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp41 in the membrane proximal region (MPER). Additional broadly neutralizing antibodies that bind to gp41 in the MPER and which can be used as the second antibody or antigen-binding fragment thereof are described, e.g., in WO 2011/034582; WO 2011/038290; WO 2011/046623 and WO 2013/070776, which are hereby incorporated herein by reference in their entireties for all purposes.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of gp41 in the membrane proximal region (MPER) and competes with or comprises VH and VL regions from an antibody selected from 10E8, 10E8v4, 10E8-5R-100cF, 4E10, DH511.11P, 2F5, 7b2, and LN01.

In some embodiments, the one or more fusion polypeptides, or polynucleotides encoding, lipoplexes (e.g., LNPs) comprising such polynucleotides, or vectors expressing such fusion polypeptides, as disclosed herein, are combined or co-administered with a broadly neutralizing antibody (bNAb)) that binds to an epitope or region of the gp41 fusion peptide and competes with or comprises VH and VL regions from an antibody selected from VRC34 and ACS202.

Examples of additional antibodies that can be co-administered include bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, DID2, 10-1074-LS, C13hmAb, GS-9722 (elipovimab), PGT-121.414, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Example of HIV bispecific and trispecific antibodies include MGD014, B12BiTe, BiIA-SG, TMB-bispecific, SAR-441236, VRC-01/PGDM-1400/10E8v4, 10E8.4/iMab, 10E8v4/PGT121-VRC01.

In some embodiments, the bNAbs can be expressed in vivo in the patient. Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

### Pharmacokinetic Enhancers

In certain embodiments, the one or more fusion polypeptides, or polynucleotides encoding, lipoplexes (e.g., LNPs) comprising such polynucleotides, or vectors expressing such fusion polypeptides, as disclosed herein, are combined with a pharmacokinetic enhancer. Examples of pharmacokinetic enhancers that can be combined or co-administered include cobicistat and ritonavir.

### Additional Therapeutic Agents

Examples of additional therapeutic agents that can be combined with the one or more fusion polypeptides, or polynucleotides encoding, lipoplexes (e.g., LNPs) comprising such polynucleotides, or vectors expressing such fusion polypeptides, as disclosed herein, include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US20100143301 (Gilead Sciences) and WO 2013/091096 (Boehringer Ingelheim).

### HIV Vaccines

In certain embodiments, the one or more fusion polypeptides, or polynucleotides encoding, lipoplexes (e.g., LNPs) comprising such polynucleotides, or vectors expressing such fusion polypeptides, as disclosed herein, are combined with an HIV vaccine. Examples of HIV vaccines that can be combined or co-administered include peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of HIV vaccines that can be co-administered include without limitation AAVLP-HIV vaccine, AE-298p, anti-CD40.Env-gp140 vaccine, Ad4-EnvC150, BG505 SOSIP.664 gp 140 adjuvanted vaccine, BG505 SOSIP. GT1.1 gp140 adjuvanted vaccine, ChAd0×1.tHIVconsv1 vaccine, CMV-MVA triplex vaccine, ChAd0×1.HTI, Chimigen HIV vaccine, ConM SOSIP.v7 gp140, ALVAC HIV (vCP1521), AIDSVAX B/E (gp120), monomeric gp 120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta] V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC—HIV-PTI, NYVAC-HIV-PT4, DNA-HIV-PT123, rAAVI-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, Maga Vax, DNA-Ad5 gag/pol/nef/nev (HVTN505), MVATG-17401, ETV-01, CDX-1401, DNA and Sev vectors vaccine expressing SCaVII, rcAD26. MOS1.HIV-Env, Ad26. Mod.HIV vaccine, Ad26. Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp 160 HIV vaccine, RNActive HIV vaccine, SCB-703, Tat Oyi vaccine, TBC-M4, UBI HIV gp120, Vacc-4x+romidepsin, variant gp 120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb 12 neutralizing VRC-01 antibody-stimulating anti-CD4 vaccine, arenavirus vector-based vaccines (VaxWave®, TheraT®), MVA-BN HIV-1 vaccine regimen, UBI HIV gp120, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined or co-administered include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In one embodiment, an agent disclosed herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir +abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXAR; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the one or more fusion polypeptides, or polynucleotides encoding, lipoplexes (e.g., LNPs) comprising such polynucleotides, or vectors expressing such fusion polypeptides, as disclosed herein, are combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In a certain embodiment, the one or more fusion polypeptides, or polynucleotides encoding, lipoplexes (e.g., LNPs) comprising such polynucleotides, or vectors expressing such fusion polypeptides, as disclosed herein, are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In another embodiment, the one or more fusion polypeptides, or polynucleotides encoding, lipoplexes (e.g., LNPs) comprising such polynucleotides, or vectors expressing such fusion polypeptides, as disclosed herein, are combined with tenofovir, tenofovir disoproxil, tenofovir disoproxil fumarate, tenofovir alafenamide, or tenofovir alafenamide hemifumarate.

In yet another embodiment, the one or more fusion polypeptides, or polynucleotides encoding, lipoplexes (e.g., LNPs) comprising such polynucleotides, or vectors expressing such fusion polypeptides, as disclosed herein, are combined with a first additional therapeutic agent selected from abacavir sulfate, tenofovir, tenofovir disoproxil, tenofovir disoproxil fumarate, tenofovir alafenamide, and tenofovir alafenamide hemifumarate, and a second additional therapeutic agent selected from emtricitabine and lamivudine.

In yet another embodiment, the one or more fusion polypeptides, or polynucleotides encoding, lipoplexes (e.g., LNPs) comprising such polynucleotides, or vectors expressing such fusion polypeptides, as disclosed herein, are combined with a first additional therapeutic agent selected from dolutegravir, cabotegravir, islatravir, darunavir, bictegravir, elsulfavirine, rilpivirine, and lenacapavir, and a second additional therapeutic agent selected from emtricitabine and lamivudine.

In another embodiment, the one or more fusion polypeptides, or polynucleotides encoding, lipoplexes (e.g., LNPs) comprising such polynucleotides, or vectors expressing such fusion polypeptides, as disclosed herein, are combined with a first additional therapeutic agent selected from tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

the one or more fusion polypeptides, or polynucleotides encoding, lipoplexes (e.g., LNPs) comprising such polynucleotides, or vectors expressing such fusion polypeptides, as disclosed herein, are combined with a first additional therapeutic agent (a contraceptive) selected from cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the one or more fusion polypeptides, or polynucleotides encoding, lipoplexes (e.g., LNPs) comprising such polynucleotides, or vectors expressing such fusion polypeptides, as disclosed herein, are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupTI cell-based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more fusion polypeptides. In some embodiments, the agents described herein are co-administered with AGT-103-transduced autologous T-cell therapy or AAV-eCD4-Ig gene therapy.

### Gene Editors

In certain embodiments, the one or more fusion polypeptides, or polynucleotides encoding, lipoplexes (e.g., LNPs) comprising such polynucleotides, or vectors expressing such fusion polypeptides, as disclosed herein, are combined with a gene editor, e.g., an HIV targeted gene editor. In various embodiments, the genome editing system can be selected from: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T-Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N-glycan on gp120, the V2 of gp120, the membrane proximal region on gp41. The immune effector cell is a T-cell or an NK cell. In some embodiments, the T-cell is a CD4+T-cell, a CD8+T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR-T-Cell Therapy

In certain embodiments, the one or more fusion polypeptides, or polynucleotides encoding, lipoplexes (e.g., LNPs) comprising such polynucleotides, or vectors expressing such fusion polypeptides, as disclosed herein, are combined with a population of TCR-T-cells. TCR-T-cells are engineered to target HIV derived peptides present on the surface of virus-infected cells, for example ImmTAV.

### B-Cell Therapy

In certain embodiments, the one or more fusion polypeptides, or polynucleotides encoding, lipoplexes (e.g., LNPs) comprising such polynucleotides, or vectors expressing such fusion polypeptides, as disclosed herein, are combined with a population of B cells genetically modified to express broadly neutralizing antibodies, such as 3BNC117 (Hartweger et al, J. Exp. Med. 2019, 1301, Moffett et al., Sci. Immunol. 4, eaax0644 (2019) 17 May 2019).

### 8. Kits

Further provided are kits comprising one or more unitary doses of one or more of the fusion polypeptides or compound fusion polypeptides, as described herein, or one or more polynucleotides encoding such fusion polypeptides or compound fusion polypeptides, as described herein, or one or more vectors expressing such fusion polypeptides or compound fusion polypeptides, as described herein. In some embodiments, the kit comprises two or more unitary doses of one or more of the fusion polypeptides or compound fusion polypeptides, as described herein, or two or more polynucleotides encoding such fusion polypeptides or compound fusion polypeptides, as described herein, or two or more vectors expressing such fusion polypeptides or compound fusion polypeptides, as described herein. In some embodiments, the one or more unitary doses are in a single container. In some embodiments, the one or more unitary doses are in two or more separate containers. In certain embodiments, the unitary doses can be the same or different, e.g., can comprise the same or different unitary doses, e.g., can comprise polypeptides, polynucleotides, vectors or combinations thereof.

In various embodiments, the kit comprises one or more pharmaceutical packs or one or more containers (e.g., vials, ampules, pre-loaded syringes) containing one or more of the ingredients of the pharmaceutical compositions described herein, such as one or more of the fusion polypeptides, as described herein, or one or more polynucleotides encoding such fusion polypeptides, as described herein, or one or more vectors expressing such fusion polypeptides, as described herein. In various embodiments, the kit comprises one or more containers comprising the one or more of the fusion polypeptides, as described herein, or one or more polynucleotides encoding such fusion polypeptides, as described herein, or one or more vectors expressing such fusion polypeptides, as described herein, in an aqueous solution. In various embodiments, the kit comprises one or more containers comprising the one or more of the fusion polypeptides, as described herein, or one or more polynucleotides encoding such fusion polypeptides, as described herein, or one or more vectors expressing such fusion polypeptides, as described herein, in lyophilized form.

In some embodiments, the kit comprises one or more unitary doses of one or more viral vectors capable of expressing the fusion polypeptides. In some embodiments, the unitary doses of the one or more viral vectors are in the range of about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In some embodiments, the kit comprises a first fusion polypeptide and a second fusion polypeptide, or one or more vectors comprising one or more polynucleotides encoding the first and second fusion polypeptides, the first and second polypeptides comprising the following polypeptide segments, in sequential order, from N-terminus to C-terminus, optionally joined or connected by one or more linkers:
SEQ ID NOs: 6, 4, 16 and 26, and SEQ ID NOs: 7, 5, 27 and 17;
SEQ ID NOs: 12, 24, 8 and 10, and SEQ ID NOs: 9, 25, 13 and 11;
SEQ ID NOs: 14, 22, 18, 24 and 20, and SEQ ID NOs: 15, 25, 19, 23 and 21;
SEQ ID NOs: 26, 16, 4 and 6, and SEQ ID NOs: 7, 27, 5 and 17;
SEQ ID NOs: 8, 24, 12 and 10, and SEQ ID NOs: 13, 25, 9 and 11;
SEQ ID NOs: 22, 24, 14, 18 and 20, and SEQ ID NOs: 25, 23, 15, 21 and 19;
SEQ ID NOs: 20, 6, 4, 18, 16 and 26, and SEQ ID NOs: 7, 19, 5, 21, 27 and 17;
SEQ ID NOs: 8, 24, 14, 12, 22 and 10, and SEQ ID NOs: 9, 13, 25, 23, 15 and 11;
SEQ ID NOs: 18, 26, 20, 4, 6 and 16, and SEQ ID NOs: 7, 21, 17, 5, 27 and 19;
SEQ ID NOs: 22, 24, 12, 14, 8 and 10, and SEQ ID NOs: 15, 25, 9, 23, 13 and 11;
SEQ ID NOs: 24, 6, 4, 20, 18 and 32, and SEQ ID NOs: 8, 30, 14, 12, 26 and 10;
SEQ ID NOs: 24, 6, 4, 20, 18 and 32, and SEQ ID NOs: 7, 21, 5, 25, 33 and 19;
SEQ ID NOs: 24, 6, 4, 20, 18 and 32, and SEQ ID NOs: 8, 30, 14, 12, 26 and 10;
SEQ ID NOs: 8, 30, 14, 12, 26 and 10, and SEQ ID NOs: 9, 13, 31, 27, 15 and 11;
SEQ ID NOs: 7, 21, 5, 25, 33 and 19 and SEQ ID NOs: 9, 13, 31, 27, 15 and 11;
SEQ ID NOs: 20, 32, 24, 4, 6 and 18, and SEQ ID NOs: 26, 30, 12, 14, 8 and 10;
SEQ ID NOs: 6, 20, 4, 24, 32 and 18, and SEQ ID NOs: 8, 12, 30, 26, 14 and 10;
SEQ ID NOs: 7, 25, 19, 5, 33 and 21, and SEQ ID NOs: 15, 31, 9, 27, 13 and 11;
SEQ ID NOs: 24, 6, 16 and 18, and SEQ ID NOs: 26, 20, 10 and 28;
SEQ ID NOs: 24, 6, 16 and 18, and SEQ ID NOs: 26, 10, 20 and 28;
SEQ ID NOs: 24, 6, 16 and 18, and SEQ ID NOs: 7, 25, 17 and 19;
SEQ ID NOs: 7, 19, 17 and 25, and SEQ ID NOs: 21, 27, 11 and 29;
SEQ ID NOs: 24, 16, 6 and 18, and SEQ ID NOs: 27, 11, 21 and 29;
SEQ ID NOs: 7, 25, 17 and 19, and SEQ ID NOs: 11, 27, 21 and 29;
SEQ ID NOs: 7, 25, 17 and 19, and SEQ ID NOs: 21, 27, 11 and 29;
SEQ ID NOs: 22, 6, 20 and 28, and SEQ ID NOs: 23, 7, 21 and 29;
SEQ ID NOs: 22, 20, 6 and 28, and SEQ ID NOs: 7, 21, 23 and 29;
SEQ ID NOs: 26, 10, 20 and 28, and SEQ ID NOs: 21, 27, 11 and 29;
SEQ ID NOs: 22, 6, 16, 20, 18, 28, 26 and 10; or.
SEQ ID NOs: 7, 21, 19, 17, 27, 25, 29 and 11.

In some embodiments, the kit comprises one or more fusion polypeptides, or one or more vectors comprising one or more polynucleotides encoding one or more fusion polypeptides, comprising or consisting of an amino acid sequence of any one of SEQ ID NOs: 70-101, 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70-101, 105-112, 200-209, 222-223 and 227. In some embodiments, the kit comprises one or more fusion polypeptides, or one or more vectors comprising one or more polynucleotides encoding one or more fusion polypeptides, comprising or consisting of an amino acid sequence of any one of SEQ ID NOs: 82-83, 85-87, 98-101, 209 and 222-223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82-83, 85-87, 98-101, 209 and 222-223.

In some embodiments, the kit comprises the following first fusion polypeptide and second fusion polypeptide, or one or more vectors comprising one or more polynucleotides encoding the following first fusion polypeptide and second fusion polypeptide, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 76 and 77, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76 and 77, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 85 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 87, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively; or
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the kit comprises first, second, third and fourth viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
b) a second viral vector comprising one or more polynucleotides encoding a first fusion polypeptide and a second fusion polypeptide comprising SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
c) a third viral vector comprising one or more polynucleotides encoding a first fusion polypeptide and a second fusion polypeptide comprising SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively; and
d) a fourth viral vector comprising one or more polynucleotides encoding a first fusion polypeptide and a second fusion polypeptide comprising SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively; and
b) a second viral vector comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively.

In some embodiments, the kit comprises first, second, third and fourth viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
b) a second viral vector comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
c) a third viral vector comprising one or more polynucleotides encoding a first fusion polypeptide and a second fusion polypeptide comprising SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 88, respectively; and
d) a fourth viral vector comprising one or more polynucleotides encoding a first fusion polypeptide and a second fusion polypeptide comprising SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively.

In some embodiments, the kit comprises first, second, third and fourth viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
b) a second viral vector comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
c) a third viral vector comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and
d) a fourth viral vector comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In some embodiments, the kit comprises first, second, third and fourth viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
b) a second viral vector comprising one or more polynucleotides encoding a first fusion polypeptide and a second fusion polypeptide comprising SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
c) a third viral vector comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively; and
d) a fourth viral vector comprising one or more polynucleotides encoding a first fusion polypeptide and a second fusion polypeptide comprising SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively, and
b) a second viral vector comprising one or more polynucleotides encoding a first fusion polypeptide and a second fusion polypeptide comprising SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding the following first fusion polypeptide and second fusion polypeptide comprising SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively, and
b) a second viral vector comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding the following first fusion polypeptide and second fusion polypeptide comprising SEQ ID NOs: 220 and 221, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 220 and 221, respectively, and
b) a second viral vector comprising one or more polynucleotides encoding the following first fusion polypeptide and second fusion polypeptide comprising SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding the following first fusion polypeptide and second fusion polypeptide comprising SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively, and
b) a second viral vector comprising one or more polynucleotides encoding the following first fusion polypeptide and second fusion polypeptide comprising SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively, and
b) a second viral vector comprising one or more polynucleotides encoding a first fusion polypeptide and a second fusion polypeptide comprising SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively.

In some embodiments, the kit comprises first and second viral vectors comprising one or more polynucleotides encoding the following first fusion polypeptide and second fusion polypeptide, optionally joined or connected by one or more linkers:
a) a first viral vector comprising one or more polynucleotides encoding a first fusion polypeptide and a second fusion polypeptide comprising SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively, and b a second viral vector comprising one or more polynucleotides encoding a first fusion polypeptide and a second fusion polypeptide comprising SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively.

In some embodiments, the kit comprises first and second viral vectors comprising polynucleotides encoding the following first compound fusion polypeptide and second compound fusion polypeptide:
a) a first viral vector comprising a polynucleotide encoding a first compound fusion polypeptide comprising SEQ ID NO: 105, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 105, and
b) a second viral vector comprising a polynucleotide encoding a first compound fusion polypeptide comprising SEQ ID NO: 109, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 109.

In some embodiments, the kit comprises first and second viral vectors comprising polynucleotides encoding the following first compound fusion polypeptide and second compound fusion polypeptide:
a) a first viral vector comprising a polynucleotide encoding a first compound fusion polypeptide comprising SEQ ID NO: 107, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 107, and
b) a second viral vector comprising a polynucleotide encoding a first compound fusion polypeptide comprising SEQ ID NO: 111, or a compound fusion polypeptide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 111.

In some embodiments, the kit comprises the following first fusion polypeptide and second fusion polypeptide, or one or more vectors comprising one or more polynucleotides encoding the following first fusion polypeptide and second fusion polypeptide, in sequential order, from N-terminus to C-terminus, optionally joined or connected by one or more linkers:
SEQ ID NOs: 70 and 71, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 70 and 71, respectively;
SEQ ID NOs: 71 and 70, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 71 and 70, respectively;
SEQ ID NOs: 72 and 73, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 72 and 73, respectively;
SEQ ID NOs: 73 and 72, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 73 and 72, respectively;
SEQ ID NOs: 74 and 75, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 74 and 75, respectively;
SEQ ID NOs: 75 and 74, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 75 and 74, respectively;
SEQ ID NOs: 76 and 77, or fusion polypeptides that are at least 80%, 81%, 82%, 97%, 98% or 99% identical to any one of SEQ ID NOs: 76 and 77, respectively;
SEQ ID NOs: 77 and 76, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 77 and 76, respectively;
SEQ ID NOs: 78 and 79, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 78 and 79, respectively;
SEQ ID NOs: 79 and 78, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79 and 78, respectively;
SEQ ID NOs: 80 and 81, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 80 and 81, respectively;
SEQ ID NOs: 81 and 80, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 81 and 80, respectively;
SEQ ID NOs: 82 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 83, respectively;
SEQ ID NOs: 83 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 82, respectively;
SEQ ID NOs: 84 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 84 and 85, respectively;
SEQ ID NOs: 85 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 84, respectively;
SEQ ID NOs: 86 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 87, respectively;
SEQ ID NOs: 87 and 86, or fusion polypeptides that are at least 80%, 81%, 82%, 97%, 98% or 99% identical to any one of SEQ ID NOs: 87 and 86, respectively;
SEQ ID NOs: 88 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 89, respectively;
SEQ ID NOs: 89 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 88, respectively;
SEQ ID NOs: 82 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 85, respectively;
SEQ ID NOs: 85 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 82, respectively;
SEQ ID NOs: 82 and 86, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 86, respectively;
SEQ ID NOs: 86 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86 and 82, respectively;
SEQ ID NOs: 82 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 82 and 88, respectively;
SEQ ID NOs: 88 and 82, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 82, respectively;
SEQ ID NOs: 83 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 83 and 87, respectively;
SEQ ID NOs: 87 and 83, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 83, respectively;
SEQ ID NOs: 85 and 87, or fusion polypeptides that are at least 80%, 81%, 82%, 97%, 98% or 99% identical to any one of SEQ ID NOs: 85 and 87, respectively;
SEQ ID NOs: 87 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 85, respectively;
SEQ ID NOs: 88 and 87, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 87, respectively;
SEQ ID NOs: 87 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 87 and 88, respectively;
SEQ ID NOs: 84 and 88, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 88 and 84, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88 and 84, respectively;
SEQ ID NOs: 85 and 89, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 89, respectively;
SEQ ID NOs: 89 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 89 and 85, respectively;
SEQ ID NOs: 85 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 85 and 101, respectively;
SEQ ID NOs: 101 and 85, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 85, respectively;
SEQ ID NOs: 90 and 91, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 90 and 91, respectively;
SEQ ID NOs: 91 and 90, or fusion polypeptides that are at least 80%, 81%, 82%, 97%, 98% or 99% identical to any one of SEQ ID NOs: 91 and 90, respectively;
SEQ ID NOs: 92 and 93, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 92 and 93, respectively;
SEQ ID NOs: 93 and 92, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 93 and 92, respectively;
SEQ ID NOs: 94 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 95, respectively;
SEQ ID NOs: 95 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 94, respectively;
SEQ ID NOs: 96 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 97, respectively;
SEQ ID NOs: 97 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 96, respectively;
SEQ ID NOs: 94 and 96, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 94 and 96, respectively;
SEQ ID NOs: 96 and 94, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 96 and 94, respectively;
SEQ ID NOs: 95 and 97, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 95 and 97, respectively;
SEQ ID NOs: 97 and 95, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 97 and 95, respectively;
SEQ ID NOs: 220 and 221, or fusion polypeptides that are at least 80%, 81%, 82%, 97%, 98% or 99% identical to any one of SEQ ID NOs: 220 and 221, respectively;
SEQ ID NOs: 221 and 220, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 221 and 220, respectively;
SEQ ID NOs: 98 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 100, respectively;
SEQ ID NOs: 100 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 98, respectively;
SEQ ID NOs: 99 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 101, respectively;
SEQ ID NOs: 101 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 99, respectively;
SEQ ID NOs: 98 and 99, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 98 and 99, respectively;
SEQ ID NOs: 99 and 98, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 99 and 98, respectively;
SEQ ID NOs: 100 and 101, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 100 and 101, respectively; or
SEQ ID NOs: 101 and 100, or fusion polypeptides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 101 and 100, respectively.

In some embodiments, the kit comprises a polynucleotide comprising or consisting of a nucleic acid sequence of any one of SEQ ID NOs: 130-167, 210-219 and 225-226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130-167, 210-219 and 225-226. In some embodiments, the kit comprises a polynucleotide comprising or consisting of a nucleic acid sequence of any one of SEQ ID NOs: 139, 140, 142, 143, 145, 146, 148, 149, 150, 152, 155, 158, 225 and 226, or a nucleic acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139, 140, 142, 143, 145, 146, 148, 149, 150, 152, 155, 158, 225 and 226.

In some embodiments, the kit comprises the following first polynucleotide and second polynucleotide, or one or more vectors comprising the following first polynucleotide and second polynucleotide:
SEQ ID NOs: 130 and 132, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 132, respectively;
SEQ ID NOs: 130 and 134, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 130 and 134, respectively;
SEQ ID NOs: 131 and 133, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 133, respectively;
SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively;
SEQ ID NOs: 132 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 132 and 136, respectively;
SEQ ID NOs: 133 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 135, respectively;
SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively;
SEQ ID NOs: 134 and 136, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 134 and 136, respectively;
SEQ ID NOs: 135 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 135 and 137, respectively;
SEQ ID NOs: 138 and 141, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 141, respectively;
SEQ ID NOs: 138 and 144, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 138 and 144, respectively;
SEQ ID NOs: 139 and 142, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 142, respectively;
SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively;
SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively;
SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively;
SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively;
SEQ ID NOs: 145 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 145 and 148, respectively;
SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively;
SEQ ID NOs: 150 and 155, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 155, respectively;
SEQ ID NOs: 151 and 153, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively;
SEQ ID NOs: 151 and 156, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 156, respectively;
SEQ ID NOs: 152 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 152 and 158, respectively;
SEQ ID NOs: 153 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 153 and 159, respectively;
SEQ ID NOs: 154 and 157, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157, respectively;
SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively;
SEQ ID NOs: 156 and 159, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively;
SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively;
SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively;
SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively;
SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively;
SEQ ID NOs: 210 and 211, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 210 and 211, respectively;
SEQ ID NOs: 212 and 213, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 212 and 213, respectively;
SEQ ID NOs: 214 and 215, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 214 and 215, respectively;
SEQ ID NOs: 216 and 217, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 216 and 217, respectively;
SEQ ID NOs: 218 and 219, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 219, respectively;
SEQ ID NOs: 218 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 218 and 226, respectively; or
SEQ ID NOs: 225 and 226, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 225 and 226, respectively.

In some embodiments, the kit comprises first and second viral vectors comprising the following first polynucleotide and second polynucleotide:
a) a first vector comprising first and second polynucleotides comprising SEQ ID NOs: 131 and 135, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively, and
b) a second vector comprising first and second polynucleotides comprising SEQ ID NOs: 133 and 137, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively. In some embodiments, the first and second viral vectors are adenoviral vectors.

In some embodiments, the kit comprises first and second viral vectors comprising the following first polynucleotide and second polynucleotide:
a) a first vector comprising first and second polynucleotides comprising SEQ ID NOs: 139 and 145, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively, and
b) a second vector comprising first and second polynucleotides comprising SEQ ID NOs: 142 and 148, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively. In some embodiments, the first and second viral vectors are adenoviral vectors.

In some embodiments, the kit comprises first and second viral vectors comprising the following first polynucleotide and second polynucleotide:
a) a first vector comprising first and second polynucleotides comprising SEQ ID NOs: 140 and 146, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively, and
b) a second vector comprising first and second polynucleotides comprising SEQ ID NOs: 143 and 149, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively. In some embodiments, the first and second viral vectors are Lymphocytic choriomeningitis mammarenavirus (LCMV) viral vectors.

In some embodiments, the kit comprises first and second viral vectors comprising

the following first polynucleotide and second polynucleotide:
a) a first vector comprising first and second polynucleotides comprising SEQ ID NOs: 150 and 152, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively, and
b) a second vector comprising first and second polynucleotides comprising SEQ ID NOs: 154 and 157 or SEQ ID NOs: 155 and 158, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively. In some embodiments, the first and second viral vectors are Cali mammarenavirus (a.k.a. Pichinde mammarenavirus or Pichinde arenavirus) viral vectors.

In some embodiments, the kit comprises first, second, third and fourth viral vectors, each vector comprising first and second polynucleotides:
a) a first viral vector comprising first and second polynucleotides comprising SEQ ID NOs: 140 and 146, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 140 and 146, respectively;

b) a second viral vector comprising first and second polynucleotides comprising SEQ ID NOs: 143 and 149, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 143 and 149, respectively;

c) a third viral vector comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively; and
d) a fourth viral vector comprising first and second polynucleotides comprising SEQ ID NOs: 154 and 157 or SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 154 and 157 or SEQ ID NOs: 155 and 158, respectively.

In some embodiments, the kit comprises first, second, third and fourth viral vectors, each vector comprising first and second polynucleotides:
a) a first viral vector comprising first and second polynucleotides comprising SEQ ID NOs: 150 and 152, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 150 and 152, respectively;
b) a second viral vector comprising first and second polynucleotides comprising SEQ ID NOs: 155 and 158, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 155 and 158, respectively;
c) a third viral vector comprising first and second polynucleotides comprising SEQ ID NOs: 151 and 153, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 151 and 153, respectively; and
d) a fourth viral vector comprising first and second polynucleotides comprising SEQ ID NOs: 156 and 159, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 156 and 159, respectively. In some embodiments, one or more of the first, second, third and fourth viral vectors are adenoviral vectors.

In some embodiments, the kit comprises first, second, third and fourth viral vectors, each vector comprising first and second polynucleotides:
a) a first viral vector comprising first and second polynucleotides comprising SEQ ID NOs: 131 and 135, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 131 and 135, respectively;
b) a second viral vector comprising first and second polynucleotides comprising SEQ ID NOs: 133 and 137, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 133 and 137, respectively;
c) a third viral vector comprising first and second polynucleotides comprising SEQ ID NOs: 139 and 145, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 139 and 145, respectively; and
d) a fourth viral vector comprising first and second polynucleotides comprising SEQ ID NOs: 142 and 148, or that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 142 and 148, respectively.

In some embodiments, the kit comprises first and second viral vectors comprising the following first polynucleotide and second polynucleotide:
a) a first vector comprising first and second polynucleotides comprising SEQ ID NOs: 160 and 161, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 160 and 161, respectively, and
b) a second vector comprising first and second polynucleotides comprising SEQ ID NOs: 162 and 163, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 162 and 163, respectively.

In some embodiments, the kit comprises first and second viral vectors comprising the following first polynucleotide and second polynucleotide:
a) a first vector comprising first and second polynucleotides comprising SEQ ID NOs: 164 and 165, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 164 and 165, respectively, and
b) a second vector comprising first and second polynucleotides comprising SEQ ID NOs: 166 and 167, or polynucleotides that are at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 166 and 167, respectively.

In some embodiments, the kit comprises first and second viral vectors comprising the following first polynucleotide and second polynucleotide:
a) a first vector comprising a first polynucleotides comprising SEQ ID NO: 210, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 210, and
b) a second vector comprising a second polynucleotide comprising SEQ ID NO: 211, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 211.

In some embodiments, the kit comprises first and second viral vectors comprising the following first polynucleotide and second polynucleotide:
a) a first vector comprising a first polynucleotides comprising SEQ ID NO: 212, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 212, and
b) a second vector comprising a second polynucleotide comprising SEQ ID NO: 213, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 213.

In some embodiments, the kit comprises first and second viral vectors comprising the following first polynucleotide and second polynucleotide:
a) a first vector comprising a first polynucleotides comprising SEQ ID NO: 214, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 214, and
b) a second vector comprising a second polynucleotide comprising SEQ ID NO: 215, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 215.

In some embodiments, the kit comprises first and second viral vectors comprising the following first polynucleotide and second polynucleotide:
a) a first vector comprising a first polynucleotides comprising SEQ ID NO: 216, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 216, and
b) a second vector comprising a second polynucleotide comprising SEQ ID NO: 217, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 217.

In some embodiments, the kit comprises first and second viral vectors comprising the following first polynucleotide and second polynucleotide:
a) a first vector comprising a first polynucleotides comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and
b) a second vector comprising a second polynucleotide comprising SEQ ID NO: 219, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 219.

In some embodiments, the kit comprises first and second viral vectors comprising the following first polynucleotide and second polynucleotide:
a) a first vector comprising a first polynucleotides comprising SEQ ID NO: 218, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 218, and
b) a second vector comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In some embodiments, the kit comprises first and second viral vectors comprising the following first polynucleotide and second polynucleotide:
a) a first vector comprising a first polynucleotides comprising SEQ ID NO: 225, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 225, and
b) a second vector comprising a second polynucleotide comprising SEQ ID NO: 226, or a polynucleotide that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 226.

In some embodiments, the kit comprises a compound fusion polypeptide or a vector comprising a polynucleotide encoding a compound fusion polypeptide, the compound fusion polypeptide comprising or consisting of an amino acid sequence of any one of SEQ ID NOs: 105-112, 200-209, 222-223 and 227, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 105-112, 200-209, 222-223 and 227. In some embodiments, the kit comprises a compound fusion polypeptide or a vector comprising a polynucleotide encoding a compound fusion polypeptide, the compound fusion polypeptide comprising or consisting of an amino acid sequence of any one of SEQ ID NOs: 209, 222 and 223, or an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 209, 222 and 223.

In various embodiments of the kits, the one or more fusion polypeptides do not comprise any polypeptide segments corresponding to Gag 54-146; Gag 370-500; Pol 1-55; Pol 118-128; Pol 321-366; Pol 432-541; Pol 607-682; Pol 709-746; Pol 828-839; Pol 921-931; Nef 1-63; Nef 100-116 and Nef 149-206, or fragments or subsequences thereof, wherein the Gag, Pol and Nef amino acid position numbers correspond to SEQ ID NOs: 1, 2 and 3, respectively. In various embodiments of the kits, the one or more fusion polypeptides do not comprise any polypeptide segments having an amino acid sequence of SEQ ID NOs: 35-47, or fragments or subsequences thereof, or an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 35-47, or fragments or subsequences thereof.

In some embodiments, the kits further comprise one or more unitary doses of one or more additional therapeutic agents. For example, in some embodiments, the kit comprises one or more agonists or activators of one or more toll-like receptors (TLRs). In some embodiments, the TLR agonist or activator is selected from a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from GS 9620 (vesatolimod), R848 (Resiquimod), DS-0509, LHC-165 and TMX-101 (imiquimod), and/or wherein the TLR8 agonist is selected from GS-9688 (Selgantolimod), R848 (Resiquimod) and NKTR-262 (dual TLR7/TLR8 agonist).

In some embodiments, the kit comprises one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL 16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7). In some embodiments, the kit comprises one or more blockers or inhibitors of one or more T-cell inhibitory immune checkpoint proteins or receptors. In some embodiments, the T-cell inhibitory immune checkpoint proteins or receptors are selected from CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In some embodiments, the kit comprises one or more agonists or activators of one or more T-cell stimulatory immune checkpoint proteins or receptors. In some embodiments, the T-cell stimulatory immune checkpoint proteins or receptors are selected from CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the kit comprises one or more blockers or inhibitors of one or more NK-cell inhibitory immune checkpoint proteins or receptors. In some embodiments, the NK-cell inhibitory immune checkpoint proteins or receptors are selected from killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In some embodiments, the kit comprises one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous inhibitor of PD-L1 (CD274), PD-1 (PDCD1) or CTLA4. In some embodiments, the proteinaceous or antibody inhibitor of CTLA4 is selected from ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In various embodiments, the co-administration of a proteinaceous or antibody inhibitor of CTLA4 increases the vaccine-induced T cell response in comparison to administration of the one or more fusion polypeptides, or polynucleotides encoding, lipoplexes (e.g., LNPs) comprising such polynucleotides, or vectors expressing such fusion polypeptides in the absence of a proteinaceous or antibody inhibitor of CTLA4. In some embodiments, the proteinaceous inhibitor of PD-L1 (CD274) or PD-1 (PDCD1) is selected from pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFß-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCD1 is selected from GS-4224, GS-4416, INCB086550 and MAX10181. In some embodiments, the small molecule inhibitor of CTLA4 comprises BPI-002.

In some embodiments, the kit comprises one or more antiviral agents. In some embodiments, the one or more antiviral agents are selected from HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, and capsid inhibitors.

Optionally associated with such container(s) can be a notice in the form prescribed by a governmental agency regulating the manufacture, use or sale of pharmaceuticals or biological products, which notice reflects approval by the agency of manufacture, use or sale for human administration.

### 9. Methods of Designing Fusion Polypeptides Useful to Promote Antiviral Immune Responses

Provided are methods for designing a vaccine construct or an immunogen that is capable of eliciting an immune response in a human against one or more viral antigens. The immunogenic fusion polypeptides are designed employing a combination of computational, experiential and manual steps, and can be used to elicit an immune response against a highly variable virus. The design methods can be applied to creating an immunogen capable of inducing an immune response in a human against one or more viral antigens of a desired target virus, including without limitation human immunodeficiency virus (HIV), hepatitis B virus (HBV), human papillomavirus (HPV), herpes simplex virus (HSV), Ebola virus, Zika virus and Chikungunya virus. Generally, the immunogen design methods provide a vaccine construct designed for maximum epitope coverage of a broad-based population, referred to herein as a “population” construct or antigen. Usually, the segments comprising each of the constructs represent one or more MHC class I and/or MHC class II T cell epitopes. The population-based polypeptide segments can be combined and assembled into immunogenic fusion polypeptides.

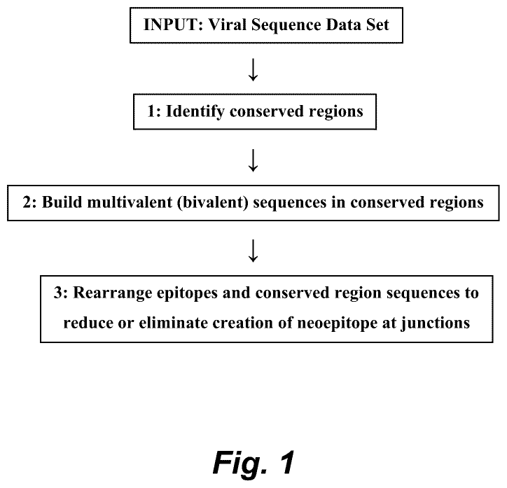

Most of the steps can be performed in silico, but some steps can be performed manually (e.g., inclusion and/or exclusion selections of certain polypeptide sequences; selection of linker or linkers) and may incorporate information based on experimental data (e.g., experimentally determined MHC class II epitopes). The input information is a viral sequence data set (e.g., for HIV, internal and publicly available HIV population data sets). As summarized in the flow chart of FIG. 1, the vaccine design methods involve at least 2, e.g., at least 3 of the steps of:
1. Identify conserved regions. All 9 amino acid segments (9-mers) are considered in naturally occurring viral sequences as potential T-cell epitopes. 9-mer positions having a conservation of at least 80% across interpatient viral populations, are identified as conserved regions and included for further analysis.
2. Build bivalent sequences from conserved regions. This can be done by employing a graph-based algorithm. 9-mers are assembled from conserved regions to include the maximum number of naturally occurring 9-mers.
3. Arrange polypeptide segments to reduce or avoid the creation of human-recognizable neoepitopes and cross-reactivities with human proteins at junctions. This can be done by evaluating 9-mers around junctions for MHC class I binding, cross-recognition with host (e.g., human, dog, cat, horse) proteins, and checking whether there are identical or substantially identical 9-mers between multiple antigen sequences if multiple insert sequences are developed simultaneously. As used herein, “substantially identical” 9-mers have at least 5 of 9 identical amino acid residues, e.g., have at least 55% (5 of 9), e.g., at least 65% (6 of 9), e.g., at least 75% (7 of 9), e.g., at least 85% (8 of 9) identical amino acid residues.

In one aspect, these methods comprise screening a set of candidate polypeptide segments of a population/conservation-based construct for MHC peptide binding affinity. MHC binding affinity can be predicted using one or more algorithms. Exemplary predictive algorithms include NetMHC (Vita et al. Nucleic Acids Res 2015 43: D405-D412), NetMHCpan (Andreatta and Nielsen Bioinformatics 2016 32:511-517), and MHCflurry (O'Donnell et al. Cell Syst 2018 7:12-132). Other T-cell epitope prediction tools are publicly available and are described, for example in Sanchez-Trincado et al. J. Immunology Res. 2017 Article ID 2680160. Additional methods for identifying MHC binding peptides include those employing machine learning tools, for example, as described in U.S. Pat. No. 10,055,540, WO 2019/104203 and the “EDGE” tool described in Bulik-Sullivan et al. Nature Biotechnology 2019 37:55.

In some implementations, the disclosure provides methods for producing a bivalent population/conservation-based construct designed both to capture the unique diversity of a viral proteome (e.g., HIV proteome) by providing mathematically determined and improved coverage of all potential T cell epitopes and to ensure that the epitopes in each polypeptide of the pair of constructed polypeptides retain the positional information of the original input viral sequences, e.g., by retaining the same order of the polypeptide segments as found in the naturally occurring viral proteome. The epitopes of the resulting pair of polypeptides will therefore more closely resemble those of the naturally occurring viral sequences, increasing the likelihood of their eliciting a relevant T cell response.

Generally, the methods described here comprise initially providing a set of mathematically determined and improved potential T cell epitopes (“PTE”) in terms of their coverage of all PTEs in a population of viral proteome sequences, using a graph-based approach. Unlike similar graph-based approaches to vaccine design, the approach described here builds segments of connected PTE's using only adjacent PTE's that are also adjacent in the natural sequences. This provides constructs that retain the positional information of the PTE's within the source set of sequences. Also, unlike other graph-based approaches, the methods described here simultaneously build a bivalent construct to provide maximal coverage of the most highly conserved PTEs in the population. The result is an initial bivalent vaccine construct that advantageously maximizes highly conserved PTEs that are most likely to be highly similar to conserved epitopes in the natural sequences. Further advantageously, the use of only the most highly conserved PTEs reduces the likelihood of escape mutants because the highly conserved sequences are more likely to be essential for viral function.

The methods described herein generally begin with the identification of a conserved region bivalent sequences, using a process referred to herein as the “Conservation Analysis” or “Conservation Algorithm”. The methods further generally comprise a step of building a bivalent vaccine construct having maximal epitope coverage while retaining the positional information of the PTE's from the natural sequences, using a process referred to referred to herein as a “Conserved Walking Algorithm” or “CWA”. Thus, in some implementations, an initial step in the method is identifying a set of all conserved regions in a viral proteome for a selected set of viral genes. In implementations for designing a fusion polypeptide to elicit an immune response against HIV-1, the set of HIV-1 viral genes is selected from two or more of Gag, Pol and Nef. In some implementations, the set of viral genes consists of Gag, Pol and Nef. In some implementations, the set of viral genes consists of Pol and Nef.

In accordance with the methods described here, a population of viral proteome sequences is first aligned to a reference sequence, for example, the HIV reference sequence HXB2 identified by GenBank No. Accession K03455. Reference sequences for polypeptides encoded by the Gag, Nef and Pol genes are provided herein as SEQ ID NOs: 1-3, in Table A. The initial input or ‘source’ population of viral proteome sequences consists of sequences obtained from naturally occurring viruses. Such sequences are publicly available, for example, from the HIV Databases maintained by the Los Alamos National Laboratory, the U.S. Dept. of Health and Human Services, and the National Institutes of Health. In some implementations of the methods described herein, the source viral sequences may consist of sequences corresponding to a specific viral group and/or clade. In some implementations, in order to improve the identification of conserved regions and sequences, the input viral sequences may be restricted to a single viral clade and/or group. In some implementations, the input viral sequences are restricted to Group M clade B sequences.

The alignment of the source viral sequences to the reference sequence may be accomplished using a multiple alignment algorithm, for example, the fast Fourier transform algorithm, MAFFT. Katoh et al. 2002 Nucleic Acids Res. 30 (14): 3059-66. The base MAFFT software is publicly available and distributed, e.g., under the Berkeley Software Distribution (BSD) license.

Next, the Conservation Algorithm is applied to the aligned sequences. For each sequence in the alignment, starting from the first amino acid position, the method shifts one amino acid position at a time and creates all possible amino acid segments that are 9 amino acids in length, referred to herein as “9-mers”. The algorithm thus creates, for each sequence in the alignment, a set of 9-amino acid subsequences (“9-mers”) starting with the N-terminal amino acid, each subsequence overlapping the preceding subsequence by eight amino acids such that each sequence of length l in the alignment contains (1-8) 9-mers.

Next, for each 9-mer position, the method identifies the two most common unique 9-mers and their prevalence in the aligned set of source viral proteome sequences. Stated another way, starting at position i the two most common unique 9-mers at each position are identified based on their frequency, calculated as the number of times the unique 9-mer occurs at position i in the alignment divided by the total number of sequences in the alignment.

Computationally, each sequence of length/, contains/-8 9-mers. We define all the 9-mers starting at position i as sᵢⱼ and frequency as fᵢⱼ, j=1,2,3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there are m+ (m-1)+ (m-2)+ . . . +2+1=m* (m+1)/2 9-mer pairs at each position.

The method then calculates the bivalent conservation for each 9-mer position by summing up the proportions of aligned set of source viral proteome sequences containing either of the two most common 9-mers. To do this, a “bivalent conservation” is calculated for each position by summing the proportion of sequences in the alignment containing either of the two most common unique 9-mers. As used herein, “bivalent conservation” refers to the percentage of sequences containing exactly the same 9 amino acid segments (9-mers) as either of the two most prevalent ones in a 9-mer position.

Next, a new alignment of conserved regions is created by extracting the sequences in the alignment having a desired bivalent conservation, for example, a bivalent conservation of greater than 80% or greater than 90%, meaning that the two most common 9-mers at position i account for more than 80% or more than 90% of the 9-mers at that position in the new alignment of conserved regions. Stated another way, the method identifies the conserved regions in the new alignment as those in which the sum of the frequencies of the two most common 9-mers at each position is greater than a certain cutoff, e.g., greater than 80% or greater than 90%. Thus, the method also calculates the frequency of each pair of unique 9-mers at each position in the new alignment of conserved regions.

In some implementations, further selection criteria may be applied to the conserved regions, such as restricting to regions having greater than 90% conservation and removing short segments of less than 35 amino acids.

Using this modified set of conserved regions, certain implementations of the method apply the CWA to build bivalent sequence constructs. The CWA connects 9-mer pairs in adjacent positions of the alignment of conserved regions that share an overlap of eight amino acids.

Computationally, each 9-mer s contains 9 amino acids, we write s [x: y] to represent the amino acid subsequence from position x to y, y-x+1 amino acids in total:

$${s}_{iu} [ 2 : 9 ] == {s}_{i + 1 p} [ 1 : 8 ] and {s}_{iv} [ 2 : 9 ] == {s}_{i + 1 q} [ 1 : 8 ]$$

$$or$$

$${s}_{iu} [ 2 : 9 ] == {s}_{i + 1 q} [ 1 : 8 ] and {s}_{iv} [ 2 : 9 ] == {s}_{i + 1 p} [ 1 : 8 ] .$$

Next, the algorithm builds a directed acyclic graph in which each 9-mer pair is a node and the edges between adjacent nodes are formed from the connected 9-mer pairs in the adjacent positions with the weight of each edge equal to the frequency of the downstream 9-mer pair. This directed acyclic graph is a positional De Brujin graph. Such graphs have been described in connection with assemblies of next generation sequencing data, for example as described in Ronen et al., Bioinformatics 2012 28:188-196. The method further adds a source node, connecting it with all of the nodes in the first position; and a sink node, connecting it with all of the nodes in the last position. The weights are then negated and the optimal path is found from the source node to the sink node, where the optimal path is defined as the path that has the largest sum of the frequencies of all 9-mer pairs among all the paths from the source node to the sink node. The task of finding the optimal path is performed, for example, using the Bellman-Ford algorithm. Generally, the Bellman-Ford algorithm computes the shortest paths from a single source vertex to all of the other vertices in a weighted directed graph which is made up of a set of vertices connected by edges, where the edges have a direction associated with them. The computational steps can be summarized as follows:
(4-1) Treat each 9-mer pair as a node, and build edges between adjacent nodes in Step 3;
(4-2) Adding a source node and connect it with all the nodes at the 1st position;
(4-3) Adding a sink node and connect it with all the nodes at the last position;
(4-4) Weight of each edge equals to the frequency of downstream 9-mer pair;
(4-5) Negating all the weights and finding the optimal path using the Bellman-Ford algorithm.

A further step of the method is to build bivalent vaccine sequences based on the optimal bivalent 9-mer pair path and connect two 9-mers in adjacent positions within the optimal bivalent 9-mer pair path if they share an overlap of 8 amino acids. A bivalent construct is built by connecting two 9-mers in adjacent positions within the optimal bivalent 9-mer path if they share an overlap of eight amino acids, thereby creating two sequences of connected 9-mers which together form the bivalent construct. The connected adjacent 9-mer pairs all have an 8 amino acid overlap, so they will be assembled into two sequences. For example, one 9-mer pair (AIIIIIIIS (SEQ ID NO: 190), MIIIIIIII (SEQ ID NO: 191)) can be connected with another 9-mer pair (IIIIIIISK (SEQ ID NO: 192), IIIIIIIIR (SEQ ID NO: 184)) and make two sequences (bivalent sequences): AIIIIIIISK (SEQ ID NO: 193) and MIIIIIIIIR (SEQ ID NO: 194).

Computationally, the methodology can be described as a positional De Brujin graph based bivalent vaccine sequence design algorithm comprising the following 5 basic steps:

Step 1: align all the population sequences

Step 2: for each 9-mer position, pull out all the unique 9-mers and their frequencies, and build 9-mer pair sets with frequencies. Each sequence of length/, contains 1-8 9-mers. We define all the 9-mers starting at position i as sᵢⱼ and frequency as fᵢⱼ, j=1,2,3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fix. Thus, in total, there are m+ (m-1)+ (m-2)+ . . . +2+1=m* (m+1)/2 9-mer pairs at each position.

Step 3: connect 9-mer pairs in adjacent positions if they do not have any conflicting amino acids. As used herein, “conflicting amino acid residues” refers to different amino acid residues at overlapped positions between two 9-mers. Each 9-mer s contains 9 amino acids, we write s [x: y] to represent the amino acid subsequence from position x to y, y-x+1 amino acids in total:

$${s}_{iu} [ 2 : 9 ] == {s}_{i + 1 p} [ 1 : 8 ] and {s}_{iv} [ 2 : 9 ] == {s}_{i + 1 q} [ 1 : 8 ]$$

$$or$$

$${s}_{iu} [ 2 : 9 ] == {s}_{i + 1 q} [ 1 : 8 ] and {s}_{iv} [ 2 : 9 ] == {s}_{i + 1 p} [ 1 : 8 ]$$

Step 4: find the optimal path from the 1st 9-mer position to the last position in terms of the sum of the frequencies of all the 9-mers within the path. The basic idea is to model the maximum coverage bivalent vaccine construction problem as a classic graph theory problem where the solution is finding the minimum path in a directed acyclic graph.

Step 5: build bivalent vaccine sequences based on the optimal bivalent 9-mer pair path and connect two 9-mers in adjacent positions within the optimal bivalent 9-mer pair path if they share an overlap of 8 amino acids. Take for example the following cases:
Case 1: if sᵢᵤ[2:9]=sᵢ₊₁ₚ [1:8] and sᵢᵥ [2:9]=s_{i+1q} [1:8], connect sᵢᵤ with sᵢ₊₁ₚ and sᵢᵥ with s_{i+1q},
Case 2: if sᵢᵤ[2:9]=s_{i+1q} [1:8] and sᵢᵥ [2:9]=sᵢ₊₁ₚ [1:8], connect sᵢᵤ with s_{i+1q} and sᵢᵥ with sᵢ₊₁ₚ;
Case 3: if sᵢᵤ[2:9]=sᵢ₊₁ₚ [1:8] and sᵢᵥ [2:9]=s_{i+1q} [1:8] and six [2:9]=s_{i+1q} [1:8] and sᵢᵥ [2:9]=Sᵢ₊₁ₚ [1:8], the selection of connection is based on the prevalence of the two connections in natural sequences:

Denote the prevalence of the co-existence of sᵢₓ and s_{i+1y} in input sequences as C_{ixy}
If Cᵢᵤₚ+C_{ivq}>C_{iuq}+Cᵢᵥₚ, connect sᵢᵤ with Sᵢ₊₁ₚ and sᵢᵥ with s_{i+1q};
If C_{iuq}+Cᵢᵥₚ>Cᵢᵤₚ+C_{ivq}, connect sᵢᵤ with s_{i+1q} and sᵢᵥ with sᵢ₊₁ₚ;

If Cᵢᵤₚ+C_{ivq}=C_{iuq}+Cᵢᵥₚ. backtrack and combine the prevalence of the co-existence of 9-mer pairs in positions i-l and i until the 1st position. If there is no difference between two different connections, randomly pick one.

In some implementations, the construct is further improved by performing a human proteome cross-recognition analysis, for example by a method comprising searching all of the 9-mers in the construct against a human proteome database such as UniProt to identify any 9-mers having a certain amino acid sequence identity with human peptides, e.g., at least 5 residues, or that share T cell receptor (TCR) facing residues with human proteins. Any such 9-mers may then be excluded from the construct. In some implementations, the polypeptide segments may optionally be rearranged to reduce or avoid deleterious junctional responses, for example by performing an HLA binding analysis, a human proteome cross-recognition analysis, or both, with respect to the junctional segments. Illustrative methods for reducing junction epitope presentation for neoantigens, in the context of designing anticancer vaccines, are described in WO 2019/104203. Alternatively, if multiple antigen sequences are developed, the polypeptide segments can be rearranged to avoid near-identical or identical 9-mers at junction between antigen sequences.

In some implementations, the conserved regions are further defined by performing one or more of the following steps in silico: (i) removing short polypeptide segments, e.g., polypeptide segments of 35 or fewer amino acids in length, e.g., 9-35 amino acids in length; (ii) removing segments that are weakly immunogenic or non-immunogenic in humans; (iii) removing segments that are less than 90% conserved, in certain instances, less than 80% conserved, amongst a predetermined population of sequences; (iv) including additional segments from HIV-1 proteins, e.g., Env, Gag, Nef and Pol, that are known to be immunogenic in humans (see, e.g., epitope maps at hiv.lanl.gov/content/immunology/maps/maps.html; Fischer, et al., Nat Med. (2007) 13 (1): 100-6; and Addo, et al., J Virol, (2003) 77 (3): 2081-92).

In some implementations, adjacent polypeptide segments may optionally be separated with a linker sequence, as described above. In some implementations, the linker sequence or sequences comprise a cleavable linker, optionally further comprising an additional linker sequence located adjacent to the cleavable linker. The additional linker may be another cleavable linker, a polyalanine linker, a polyglycine linker, a flexible linker, or a rigid linker, as described above and herein. In some embodiments, a furin recognition site precedes or is positioned N-terminal to a 2A cleavable linker. In a specific implementation, where the linker sequence comprises a foot-and-mouth disease virus (FMDV) cleavable peptide, FMDV 2A, or derivative thereof, the additional linker sequence may be a REKR (SEQ ID NO: 61) sequence, or derivative thereof. In some implementations, the linker is selected from a short polyalanine peptide, for example a peptide consisting of from 2 to 4 alanine residues, or having the sequence AAY (SEQ ID NO: 49) or AAX, where X is any amino acid residue (SEQ ID NO: 50).

In some implementations, the linker is inserted between each adjacent conserved region of the bivalent polypeptide. In some implementations, e.g., when no deleterious junctional epitope is created, no linker is inserted between adjacent segments of the same protein in the polypeptide. A linker can be inserted between adjacent segments of different proteins.

### EXAMPLES

The following examples are offered to illustrate, but not to limit the claimed invention.

### Example 1

### Illustrated Implementation of the Conservation Analysis and Conserved Walking Analysis (CWA) to Generate a Bivalent Vaccine Construct

This Example describes the design of population-based bivalent polypeptide constructs by a specific implementation of the Conservation Analysis and CWA to generate a bivalent vaccine construct based on conserved protein regions encoded by the HIV-1 Gag, Nef and/or Pol genes.

First, we identified a set of all conserved regions in a viral proteome for a selected set of viral genes. In this example, the set of viral genes consisted of HIV-1 Gag, Pol, and Nef.

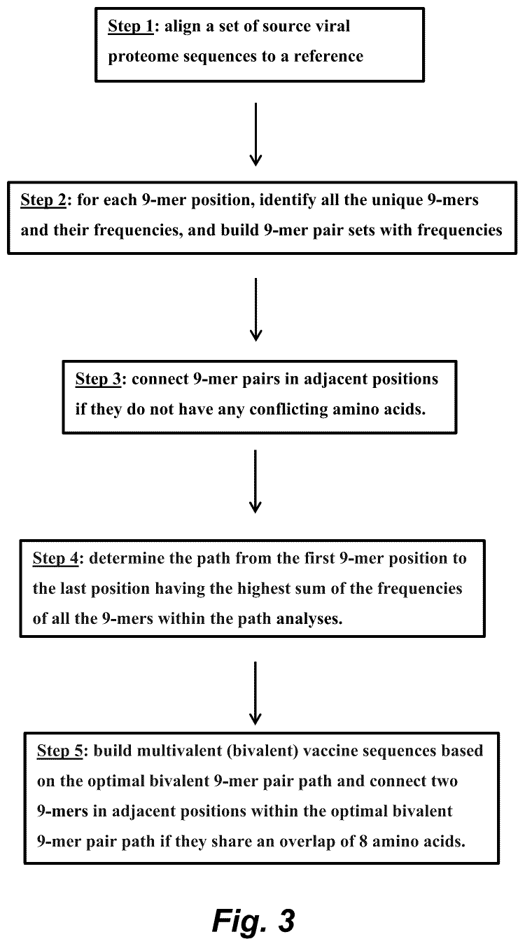

Computationally, the combination of the Conservation Algorithm and the CWA is a positional De Brujin graph based bivalent vaccine sequence design algorithm comprising the following 5 basic steps, illustrated in FIG. 3:

### Step 1: Align a Set of Source Viral Proteome Sequences to a Reference Sequence.

In Step 1, a source population of viral proteome sequences was aligned to a reference sequence. In this example, the reference sequence used was the HIV-1 HXB2, identified by GenBank No. Accession K03455. The amino acid sequences of HXB2 reference polypeptides Gag, Nef and Pol are provided herein as SEQ ID NOs: 1, 2 and 3, respectively, in Table A. The source population of viral proteome sequences consists of sequences obtained from naturally occurring viruses. Such sequences are publicly available, for example, from the HIV Databases maintained by the Los Alamos National Laboratory, the U.S. Dept. of Health and Human Services, and the National Institutes of Health (hiv.lanl.gov), which was the database used for the source population of sequences in this example. For the purposes of illustration, we focused our analysis on a subset of the viral sequences, here, sequences of Group M Clade B. The alignment was performed using a multiple alignment algorithm, specifically a fast Fourier transform algorithm, MAFFT. Katoh, et al. (2002) Nucleic Acids Res. 30 (14): 3059-66. The base MAFFT software is publicly available and distributed, e.g., under the Berkeley Software Distribution (BSD) license.

Step 2: For Each 9-Mer Position, Pull Out all the Unique 9-Mers and their Frequencies, and Build 9-Mer Pair Sets with Frequencies

In Step 2, we applied the Conservation Algorithm to the set of aligned sequences. For each sequence in the alignment, starting from the first amino acid of the N-terminus, the algorithm shifts one amino acid position at a time to create a set of all possible amino acid segments that are 9 amino acids in length, referred to as “9-mers.” The algorithm thus created, for each sequence in the alignment, a set of 9-amino acid subsequences (“9-mers”) starting with the N-terminal amino acid, each subsequence overlapping the preceding subsequence by eight amino acids such that each sequence of length/in the alignment contains (1-8) 9-mers.

Next, for each 9-mer position, the method identified the two most common unique 9-mers and their prevalence in the aligned set of source viral proteome sequences. Stated another way, starting at position i the two most common unique 9-mers at each position were identified based on their frequency, calculated as the number of times the unique 9-mer occurred at position i in the alignment divided by the total number of sequences in the alignment.

Computationally, each sequence of length/, contained/-8 9-mers. We defined all the 9-mers starting at position i as sy and frequency as fᵢⱼ, j=1,2,3, . . . m. In total there were m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there were m* (m+1)/2 9-mer pairs at each position.

The method then calculated the bivalent conservation for each 9-mer position by summing up the proportions of aligned set of source viral proteome sequences containing either of the two most common 9-mers. To do this, a “bivalent conservation” was calculated for each position by summing the proportion of sequences in the alignment containing either of the two most common unique 9-mers.

Next, a new alignment of conserved regions was created by extracting the sequences in the alignment having a desired bivalent conservation. In this example, we used a bivalent conservation of greater than 80% or greater than 90%, meaning that the two most common 9-mers at position i accounted for more than 80% or more than 90% of the 9-mers at that position in the new alignment of conserved regions. Stated another way, the method identified the conserved regions in the new alignment as those in which the sum of the frequencies of the two most common 9-mers at each position was greater than a certain cutoff, e.g., greater than 80% or greater than 90%. Thus, the method also calculated the frequency of each pair of unique 9-mers at each position in the new alignment of conserved regions.

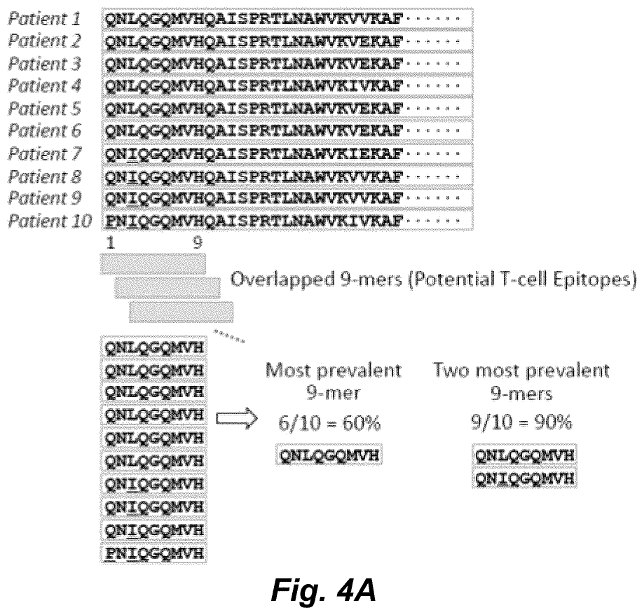

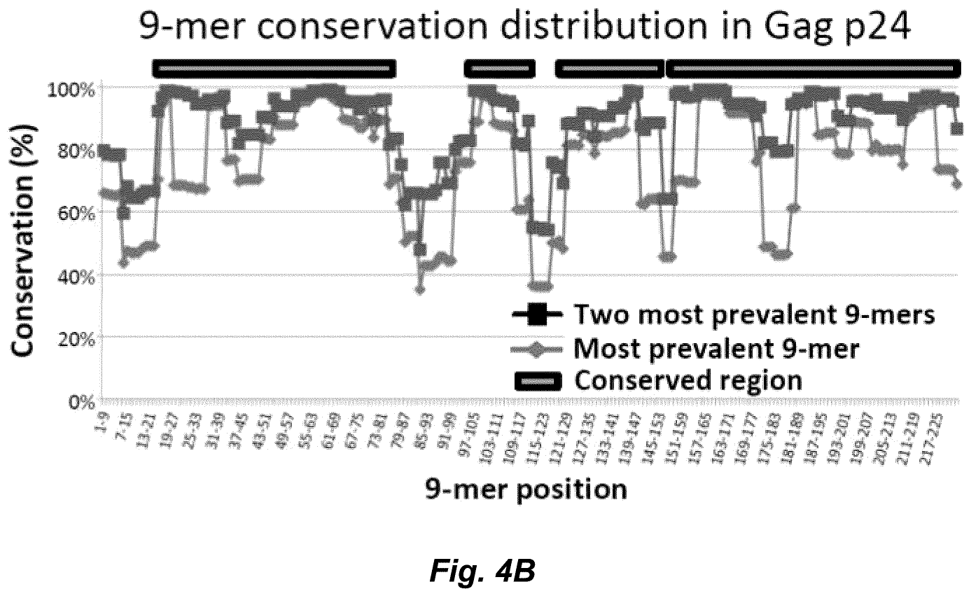

This is illustrated graphically in FIG. 4A. FIG. 4A shows a hypothetical set of 10 input natural sequences, each having a single amino acid variation within the first 9-mer. Across the set of 10 sequences, the 9-mer having an “L” at the third amino acid position occurs 6 times, the 9-mer having an “I” at that location occurs 3 times, and the 9-mer having an “I” at that location but a different amino acid in the first position occurs once. Thus, the Conservation Algorithm selected the two most prevalent 9-mers which together account for 90% of the possible 9-mers at that position in the population of aligned sequences.

Using this analysis, the distribution of highly conserved 9-mers at each position across all of the protein sequences in the population was determined. This is illustrated graphically in FIG. 4B. The plot shows the conservation distribution for proteins encoded by the Gag gene p24 protein in 9,846 Group M Clade B input sequences obtained from the Los Alamos HIV Sequence database. The y-axis shows bivalent conservation and the x-axis shows the location of the 9-mer relative to the reference sequence, Gag p24 from HXB2. Across the top of the graph the horizontal bars depict conserved regions as those having at least 80% bivalent conservation using the two most prevalent 9-mers at each position. The dark gray line with the squares plots the bivalent conservation at each position using the two most prevalent 9-mers while the light gray line with the diamonds shows conservation using only the most prevalent 9-mer at each position. This analysis demonstrates that the use of the two most prevalent 9-mers improved the identification of structurally conserved sequences with an input population.

We next applied further selection criteria to define the conserved regions, including restricting to regions having greater than 90% bivalent conservation and removing short segments of less than 35 amino acids, e.g., segments 9-35 amino acids in length.

We also included some additional segments from certain regions having at least 80% bivalent conservation and known to be highly immunogenic, in particular, the region of Nef corresponding to amino acids 64-99 of the reference sequence HXB2_K03455 (see, e.g., epitope maps at hiv.lanl.gov/content/immunology/maps/maps.html; Fischer, et al., Nat Med. (2007) 13 (1): 100-6; and Addo, et al., J Virol, (2003) 77 (3): 2081-92).

Step 3: Connect 9-Mer Pairs in Adjacent Positions if they do not have any Conflicting Amino Acids.

Using this modified set of conserved regions, we applied the CWA to build bivalent sequence constructs. The CWA connects 9-mer pairs in adjacent positions of the alignment of conserved regions that share an overlap of eight amino acids.

Computationally, each 9-mer s contains 9 amino acids, and s [x: y] represented the amino acid subsequence from position x to y, y-x+1 amino acids in total:

$${s}_{iu} [ 2 : 9 ] == {s}_{i + 1 p} [ 1 : 8 ] and {s}_{iv} [ 2 : 9 ] == {s}_{i + 1 q} [ 1 : 8 ]$$

$$or$$

$${s}_{iu} [ 2 : 9 ] == {s}_{i + 1 q} [ 1 : 8 ] and {s}_{iv} [ 2 : 9 ] == {s}_{i + 1 p} [ 1 : 8 ]$$

Step 4: Find the Optimal Path from the First 9-Mer Position to the Last Position in Terms of the Sum of the Frequencies of all the 9-Mers within the Path.

In Step 4, the algorithm built a directed acyclic graph in which each 9-mer pair was a node and the edges between adjacent nodes were formed from the connected 9-mer pairs in the adjacent positions with the weight of each edge equal to the frequency of the downstream 9-mer pair. This directed acyclic graph is a positional De Brujin graph. Such graphs have been described in connection with assemblies of next generation sequencing data, for example as described in Ronen et al., Bioinformatics (2012) 28:188-196.

In the present example, we added a source node and connected it with all of the nodes in the first position; and we added a sink node and connected it with all of the nodes in the last position. In a directed graph, a source node was a node that only has out flow and a sink node was a node that only has in flow. Here, the source node was a dummy node that connects to all the 9-mer pair nodes in the first position, and the sink node was a dummy node that connects to all the 9-mer pair nodes in the last position.

We then negate all of the weights and find the optimal path from the source node to the sink node, where the optimal path is defined in terms of the sum of the frequencies of all 9-mer pairs. The task of finding the optimal path is performed, for example, using the Bellman-Ford algorithm. Generally, the Bellman-Ford algorithm computes the shortest paths from a single source vertex to all of the other vertices in a weighted directed graph. A directed graph is one made up of a set of vertices connected by edges, where the edges have a direction associated with them.

Computationally, the basic idea was to model the maximum coverage bivalent vaccine construction problem as a classic graph theory problem where the solution was finding the minimum path in a directed acyclic graph. The computational steps are summarized as follows:
(4-1) Treat each 9-mer pair as a node, and build edges between adjacent nodes in Step 3;
(4-2) Adding a source node and connect it with all the nodes at the 1st position;
(4-3) Adding a sink node and connect it with all the nodes at the last position;
(4-4) Weight of each edge equals to the frequency of downstream 9-mer pair; and
(4-5) Negating all the weights and finding the optimal path using the Bellman-Ford algorithm.
   Step 5: Build Bivalent Vaccine Sequences Based on the Optimal Bivalent 9-Mer Pair Path and Connect Two 9-Mers in Adjacent Positions within the Optimal Bivalent 9-Mer Pair Path if they Share an Overlap of 8 Amino Acids.

In Step 5, a bivalent construct was built by connecting two 9-mers in adjacent positions within the optimal bivalent 9-mer path if they shared an overlap of eight amino acids, thereby creating two sequences of connected 9-mers which together form the bivalent construct. The connected adjacent 9-mer pairs all had an 8 amino acid overlap, so they were assembled into two sequences. For example, one 9-mer pair (AIIIIIIIS (SEQ ID NO: 190), MIIIIIIII (SEQ ID NO: 191)) was connected with another 9-mer pair (IIIIIIISK (SEQ ID NO: 192), IIIIIIIIR (SEQ ID NO: 184)) and made two sequences (bivalent sequences): AIIIIIIISK (SEQ ID NO: 193) and MIIIIIIIIR (SEQ ID NO: 194).

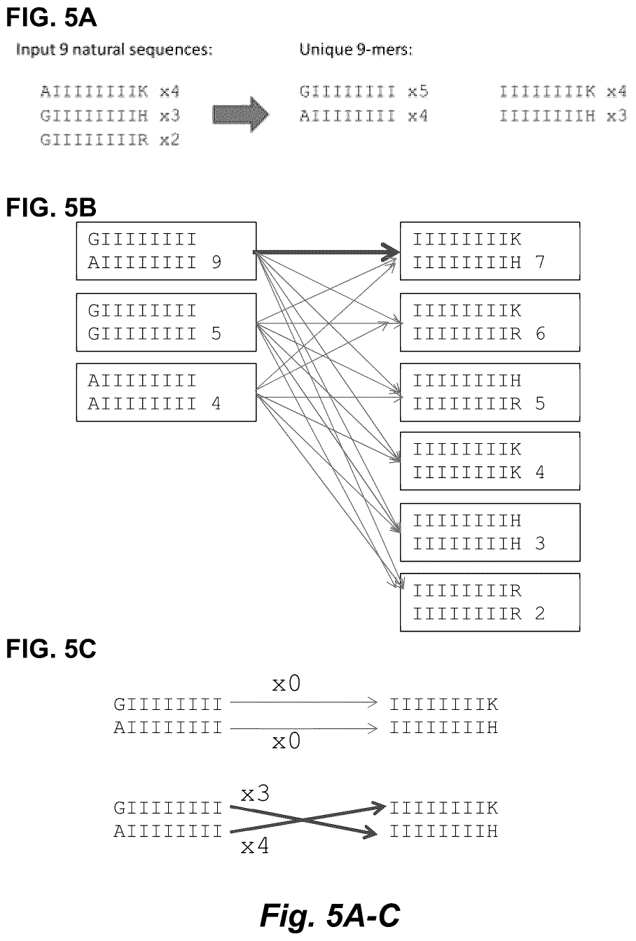

This method is illustrated graphically in FIGS. 5A-5C. FIG. 5A shows a hypothetical set of 9 source viral sequences having, at the first position, 2 unique 9-mers and at the second adjacent position 3 unique 9-mers. The frequency of each sequence is indicated to the right of the sequence as ‘times’, e.g., “x5” means that sequence occurs 5 times in the source set. FIG. 5B depicts the building of the positional De Brujin graph in which each node is one bivalent 9-mer pair. Where two bivalent 9-mer pairs in adjacent positions shared an overlap of eight amino acids they were connected to build an edge. In this manner the directed acyclic graph was created. FIG. 5C illustrates the finding of the optimal path. As noted above, the optimal path was defined in terms of the sum of the frequencies of all 9-mer pairs. This was accomplished by finding the connection between adjacent 9-mers that provided the highest conservation with reference to the input sequences. Thus, in FIG. 5C, connecting the two 9-mer pairs as shown in the top set of four pairs provided the following bivalent sequences,

(SEQ ID NO: 195)
GIIIIIIIIK
x0
(SEQ ID NO: 196)
AIIIIIIIIH
x0.

Neither of these sequences was present in the source sequences shown in FIG. 5A.

In contrast, connecting the two 9-mer pairs as shown in the bottom set of four pairs in FIG. 5C provided the following bivalent sequences,

(SEQ ID NO: 178)
GIIIIIIIIH
x3
(SEQ ID NO: 177)
AIIIIIIIIK
x4.

Each of these was present, 3 or 4 times, respectively, in the source sequences shown in FIG. 5A. Accordingly, it is these second pairs of bivalent sequences that were selected by the algorithm because it maximized conservation relative to the source sequences.

Computationally, this can be illustrated by the following exemplary cases:
Case 1: if sᵢₓ [2:9]=sᵢ₊₁ₚ [1:8] and sᵢᵥ [2:9]=s_{i+1q} [1:8], connect sᵢₓ with sᵢ₊₁ₚ and sᵢᵥ with s_{i+1q};
Case 2: if sᵢₓ [2:9]=s_{i+1q} [1:8] and sᵢᵥ [2:9]=sᵢ₊₁ₚ [1:8], connect sᵢₓ with s_{i+1q} and sᵢᵥ with sᵢ₊₁ₚ;
Case 3: if sᵢₓ [2:9]=sᵢ₊₁ₚ [1:8] and sᵢᵥ [2:9]=s_{i+1q} [1:8] and sᵢₓ [2:9]=s_{i+1q} [1:8] and sᵢᵥ [2:9]=si+1p [1:8], the selection of connection is based on the prevalence of the two connections in natural sequences:

Denote the prevalence of the co-existence of sᵢₓ and si+ly in input sequences as C_{ixy}
If Cᵢᵤₚ+C_{ivq}>C_{iuq}+Cᵢᵥₚ, connect sᵢᵤ with sᵢ₊₁ₚ and sᵢᵥ with s_{i+1q};
If C_{iuq}+Cᵢᵥₚ>Cᵢᵤₚ+C_{ivq}, connect Sin with s_{i+1q} and sᵢᵥ with sᵢ₊₁ₚ;

If Cᵢᵤₚ+C_{ivq}=C_{iuq}+Cᵢᵥₚ. backtrack and combine the prevalence of the co-existence of 9-mer pairs in positions i−1 and i until the first position. If there is no difference between two different connections, randomly pick one.

This backtrack and co-existence prevalence approach considered prevalence of peptides longer than 9 amino acids and further differentiated the present algorithm from other graph-based methods.

Next, constructed sequences from regions not adjacent to one another in the natural sequence, that is, regions which could not be joined according to the CWA as described above due to their lacking an 8 amino acid overlap, were combined using one of three different linker strategies: 1. direct fusion without any linker; 2. insert ‘AAA’ (SEQ ID NO: 48) linker between each two conserved regions; 3. direct fusion without any linker for segments within the same protein and insertion of an F2A linker between segments from different proteins.

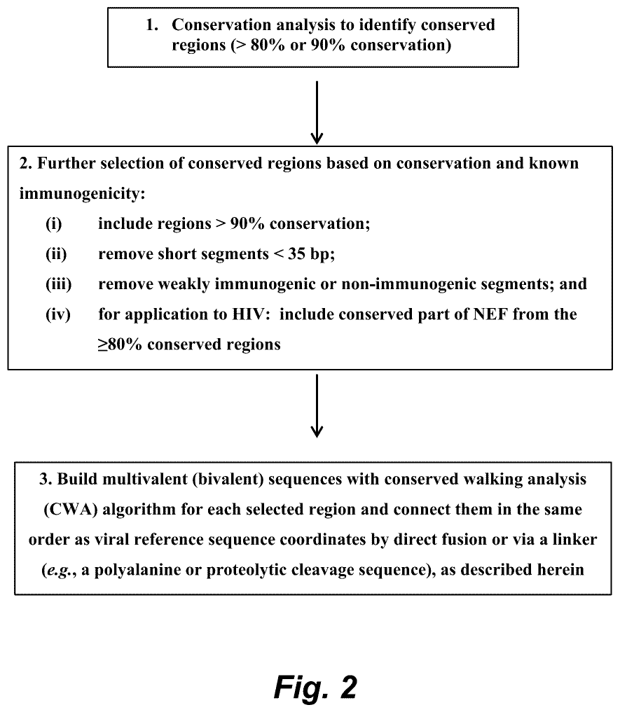

An overview of the Conserved Walking Analysis (CWA) method is shown in FIGS. 1 and 2.

### Example 2

### Illustrated Implementation of the Conservation Analysis and Conserved Walking Analysis (CWA) Applied to Proteins Encoded by HIV-1 Genes

This example describes a similar implementation based on conserved HIV-1 regions of (1) Gag, Pol and Nef or (2) Pol and Nef.

In Example 1 above, the Conservation algorithm was applied to identify a set of all candidate conserved regions in the protein coding regions of the target genes Gag, Nef and Pol. In this example, we utilized the protein coding regions of (1) Gag, Pol and Nef, (2) Pol and Nef to generate different bivalent constructs. As in Steps 1-2 of Example 1 above, we first aligned the source sequences and then applied the Conservation Algorithm to identify a set of all candidate conserved regions in the protein coding regions of the target genes, which were either from (1) Gag and Nef, Pol, or (2) Pol and Nef. As above, we then we applied further selection criteria based on conservation and known immunogenicity (see, e.g., epitope maps at hiv.lanl.gov/content/immunology/maps/maps.html and Fischer, et al., Nat Med. (2007) 13 (1): 100-6). In certain sequences including polypeptide segments encoding by the Pol gene, we excluded sequence segments including one or both of the “YMDD” motif (SEQ ID NO: 197) in reverse transcriptase and the “DTG” motif in protease, because they may affect one or both of the expression and maintenance of enzymatic activity.

Using this modified set of conserved regions, we applied the CWA to build bivalent sequence constructs, as in Steps 3-5 in Example 1.

Some polypeptide segments were connected by a polyalanine linker (e.g., AA, AAA (SEQ ID NO: 48) or AAY (SEQ ID NO: 49)), chosen for demonstration purposes because it is a small flexible linker that is unlikely to have a significant influence on protein structure. Some polypeptide segments were connected by natural short linkers (e.g., K, I, LI, EE, PPV, LIK, KIL, QEE, or SEG), chosen from the natural HIV protein sequences that flanking the conserved polypeptide segments to be fused. If we determined that it was possible to fuse polypeptide segments without creating a deleterious or undesirable junctional epitope, e.g., such as one that may stimulate T cells that may cross react to self-antigens, a fusion approach was used. If we determined that a deleterious or undesirable junctional epitope may be created, a flexible or natural short linker was inserted between polypeptide segments.

For this Example, we applied a further analysis of the junctional regions for possible presentation of deleterious epitopes and arranged the segments to reduce or avoid the creation of such junctional epitopes.

Different arrangements of peptide segments generate different junction 9-mers that can induce different junction responses. We developed a polypeptide segment arrangement tool to examine MHC binding affinities and cross-recognition with human peptides for all the junction 9-mers in each arrangement. Our internally developed polypeptide segment arrangement tool searched different arrangements of peptides and determined the best arrangement with minimal junction response based on in silico prediction results of applying the two analyses described below ((1) in-silico HLA binding analysis and (2) human proteome analysis to identify epitopes that may prime T cells that may recognize self-antigens) on the junctions of 9-mers. The junctional response score between each two adjacent segments was determined by the sum of the number of junction 9-mers that were predicted to have high binding affinities to target HLA alleles and the number of human proteins predicted to have peptides or T cell recognition motifs with any junction 9-mers. The score of each segment arrangement was determined by the sum of the junctional response scores for all the junctional regions in each segment arrangement.
1) When there were less than 15 peptide segments, our internally developed polypeptide segment arrangement tool searched all the possible arrangements and determined the best one with minimal junction response (the lowest segment arrangement score).
2) When there were at least 15 peptide segments, our internally developed polypeptide segment arrangement tool uses a ‘greedy’ strategy. It first created all the junctions and then started from the best junction in terms of predicted junctional response. Next, it searched for the next compatible best junction iteratively and assembled all the peptide segments.

In-silico MHC class I (human HLA) binding analysis: Antigen processing, presentation, and T cell receptor recognition are complex processes that remain incompletely understood. Intracellular and extracellular antigens are processed within endosomal compartments, and the cytoplasm by the proteasome and trafficked to endosomal compartments such as the ER where they peptide fragments interact with MHC molecules. Stable peptide-MHC complexes are trafficked to the cell surface where they can be recognized by a T cell expressing a TCR with the appropriate specificity. One of the most selective steps in antigen processing and presentation is HLA binding. HLA binding affinities can be predicted using various tools such as NetMHC or MHCflurry, or large internal datasets derived from immunopeptidome analyses and confirmed by experimental binding data as well as epitopes defined from patient samples. These tools are publicly available and are described, for example, in Lundegarrd et al., Nucleic Acids Res. 2008 Jul. 1; 36 (Web Server issue): W509-12 and O'Donnell, et al., Cell Systems 2018 7:129-132. In this example we used NetMHC, version 4.0. The default settings were used for all the parameters in NetMHC, along with inputting information for peptide sequences and HLA alleles. Predicted binding affinities with an IC50 value less than 1,000 nM were considered as low binding affinities.

Human proteome cross-recognition analysis: Epitopes similar to human peptides may induce tolerogenic responses or responses that may cross-react with self-antigens. We searched all the 9-mers in our vaccine against public human protein databases (e.g., Uniprot, NCBI). If an HIV peptide 9-mer has at least a 5-residue amino acid sequence identity with a human peptide 9-mer, and both were predicted to have high binding affinities to the same alleles, they were considered as cross-conserved 9-mers. We downloaded all the human protein sequences from the UniProt database and built a tool to support efficient search of a given 9-mer against all the human protein 9-mers with up to 4 mismatches (at least 5 matches).

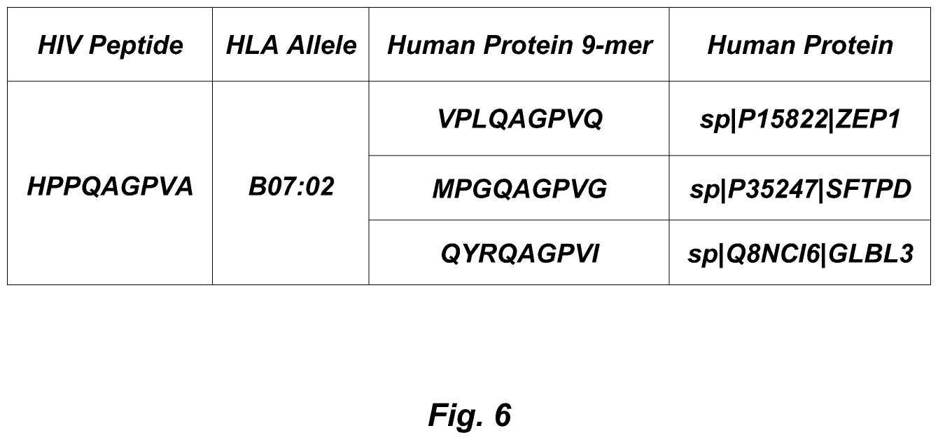

FIG. 6 illustrates the results of human proteome cross-recognition analysis. In this example, we searched HIV-1 peptide 9-mers over human protein databases and identified all the human protein 9-mers sharing a certain number of amino acids (at least 5 tentatively) and were predicted to have high binding affinities (e.g., IC50 of less than about 1000 nM or having a percentile rank within the top 5% in a population of polypeptide segments) to the same alleles based on the in silico MHC class I analysis described herein. Such HIV 9-mers having both substantial or high sequence identity (e.g., having at least 55% (5 of 9 amino acid residues), e.g., at least 65% (6 of 9 amino acid residues), e.g., at least 75% (7 of 9 amino acid residues), e.g., at least 85% (8 of 9 amino acid residues)) to a peptide segment of a human protein and high predicted MHC class I binding affinity were excluded because they may induce tolerogenic responses or responses that may cross-react with human self-antigens (defined herein as “deleterious epitopes”).

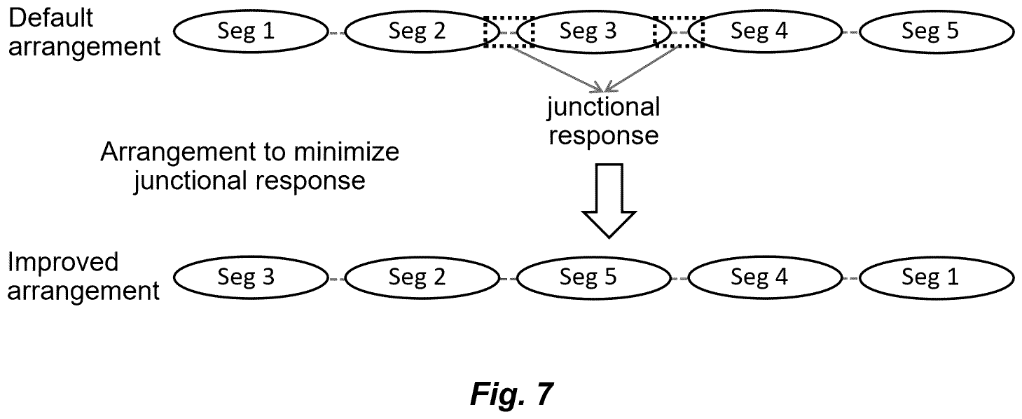

FIG. 7 illustrates how polypeptide segment arrangement analysis can reduce or eliminate possible presentation of deleterious or undesirable epitopes in junction regions. In the illustrated default arrangement, the junction 9-mers between Seg 2 and Seg 3, and between Seg 3 and Seg 4 were predicted to produce junctional sequences that may induce tolerogenic or self-reactive responses in a human (e.g., having either high MHC binding affinity based on in silico HLA binding analysis or cross-recognition with human proteins based on human proteome cross-recognition analysis). We applied an algorithm that searched different arrangements and determined an arrangement that results in reduced or eliminated predicted junctional sequences that may induce tolerogenic or self-reactive responses in a human. Additionally, if multiple antigen sequences were developed, the polypeptide segments were also rearranged to avoid identical or nearly identical 9-mers at junction between antigen sequences.

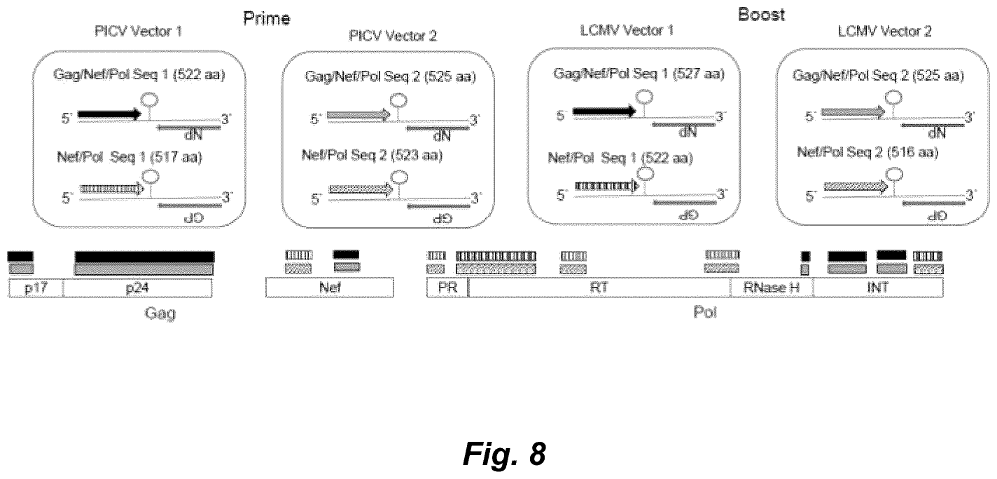

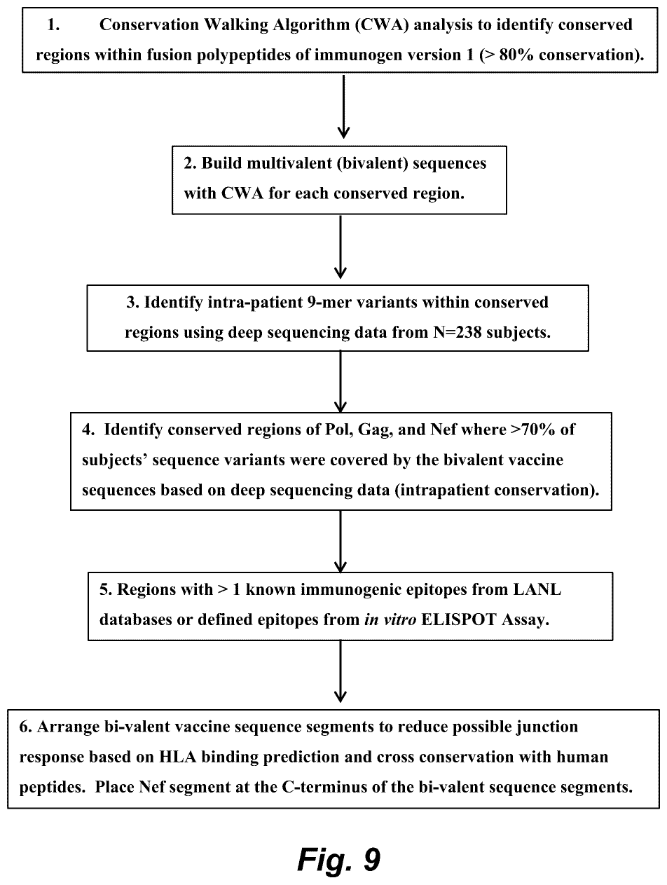

The fusion polypeptides and compound fusion polypeptides described herein are exemplary immunogenic fusion polypeptide sequences designed according to the herein described immunogen design methods. The fusion polypeptides of SEQ ID NOs: 70-101 (provided in Table E), particularly SEQ ID NOs: 94-101 (immunogen version 1), and the compound fusion polypeptides of SEQ ID NOs: 105-112, 200-209, 222-223 and 227 (provided in Table F), which have polypeptide segments encoding by the HIV-1 Gag, Nef and Pol genes, are exemplary immunogenic fusion polypeptide sequences designed according to this method. FIG. 8 provides a schematic of viral vectors containing the fusion proteins of immunogen version 1.

### Example 3

Immunogen Design Improved with Deep sequencing Analysis and Immunogenicity Data: Immunogen Version 2

In Example 1 above, the Conservation algorithm was applied to identify a set of all candidate conserved regions in the protein coding regions of the target genes Gag, Nef and Pol. In Example 2, we utilized the protein coding regions of (1) Gag, Pol and Nef, (2) Pol and Nef to generate different bivalent constructs. In this example, we describe the design of immunogen version 2 by incorporating both deep sequencing data and immunogenicity data to identify which conserved regions from immunogen version 1 to be included in shortened bivalent polypeptide constructs. For the design of this improved immunogen, as in Steps 1-2 of Example 1 above, we first aligned the source sequences and then applied the herein described conserved walking algorithm (CWA) to identify a set of all candidate conserved regions in the protein coding regions of the target genes. The target genes were Gag, Nef and Pol, and we applied the CWA to build bivalent sequences in those regions, as in Steps 3-5 of Example 1.

INTRA-patient Conservation Analysis using Deep Sequencing. In addition to the 9-mers derived from downloaded population sequences, we also analyzed viral deep sequencing data from HIV-1 subtype B chronically infected individuals to identify intra-patient diversity within those conserved regions. To identify intra-patient 9-mer variants using deep sequencing data, deep sequencing data of N=238 HIV chronically infected treatment-naïve subjects were analyzed to evaluate intra-patient diversity within conserved regions defined by CWA. To evaluate intra-patient 9-mer variants using deep sequencing data, deep sequencing reads were assembled to create subject-specific consensus sequence. Reads were aligned to subject-specific consensus sequence and then the alignment was mapped to HXB2 position coordinates based on alignment of subject-specific consensus to HXB2 reference sequence. At each 9-mer position, corresponding sequencing reads completely covering the 27 bp of the 9-mer were extracted and converted into 9-mer amino acid sequences. Only 9-mer positions with ≥1000 sequencing read depth were included in the analysis and variants were analyzed with a 1% frequency cutoff. The 9-mer variants were used to evaluate the bivalent vaccine sequences for coverage of quasi-species variants. Here we defined the intra-patient conservation of a 9-mer position as percentage of patients (Total N=238 samples) that were covered by the bivalent vaccine sequences without escape variants. High intra-patient conserved 9-mers are the 9-mers that were covered by bivalent population-based vaccine sequences without any escape variants in >70% patients. The intra-patient conservation was evaluated for the regions of Pol, Gag, and Nef that were selected based on population conservation as described in Example 1.

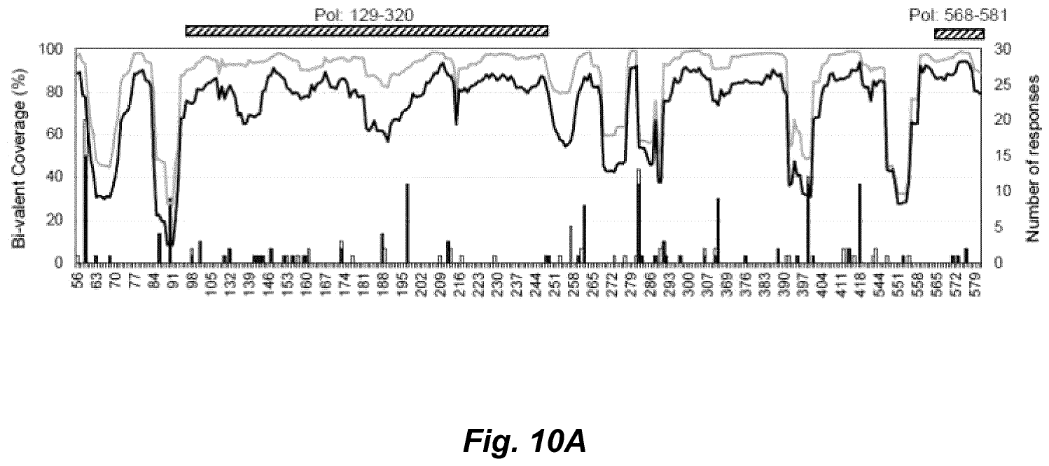

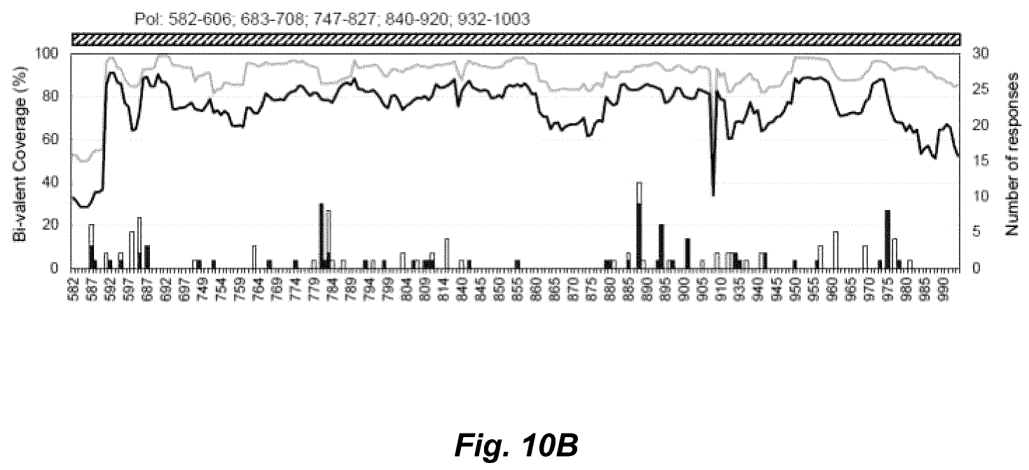

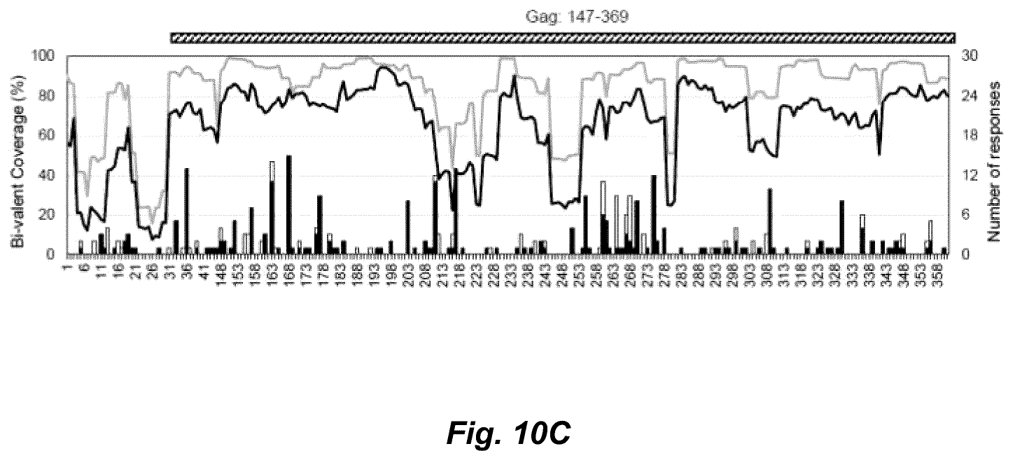

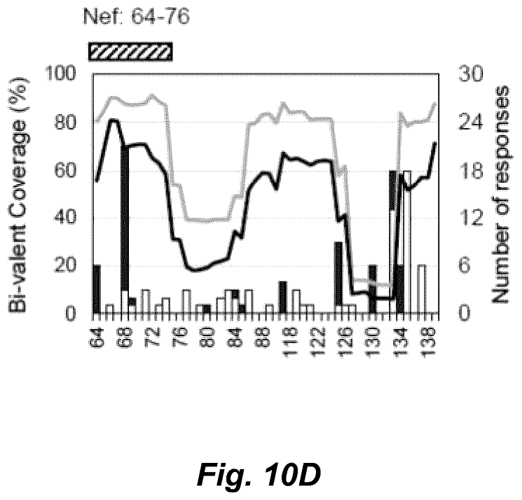

FIG. 10 provides a flow diagram illustrating the basic methodology of the approach for designing immunogen version 2. The fusion polypeptides of SEQ ID NOs: 82-89 are illustrative amino acid sequences designed according to this method.

Known epitopes from Los Alamos National Laboratory (LANL) database and Gilead ELISPOT Assay Identified Epitopes. To evaluate immunogenicity of the previously identified conserved regions, described CD8+T-cell epitopes (e.g., from LANL database) and internally conducted ELISpot assays were analyzed. Any 9-mer positions in conserved regions of Pol, Gag, and Nef with >1 mapped epitope defined by ELISPOT assay or from LANL database were considered as an immunogenic region. Immunogenicity results were not limited to specific HLA alleles.

FIGS. 10A-10C illustrate the regions that were selected for immunogen version 2 by combining deep sequencing data and immunogenicity data. The intra-patient conservation is represented as the black horizontal lines for each 9-mer position in the plot. The number of epitopes overlapped at each 9-mer region is shown as stacked bar in the plot. We selected the amino acid 9-mers that have intra-patient conservation >70% and overlap with epitopes reported by LANL database or defined by ELISpot assay. After identifying the amino acid 9-mers to use for the improved immunogen, those 9-mers that were from the same viral protein and contiguous were connected (e.g., via a linker or directly fused) to form fusion amino acid segments as highlighted in the plot.

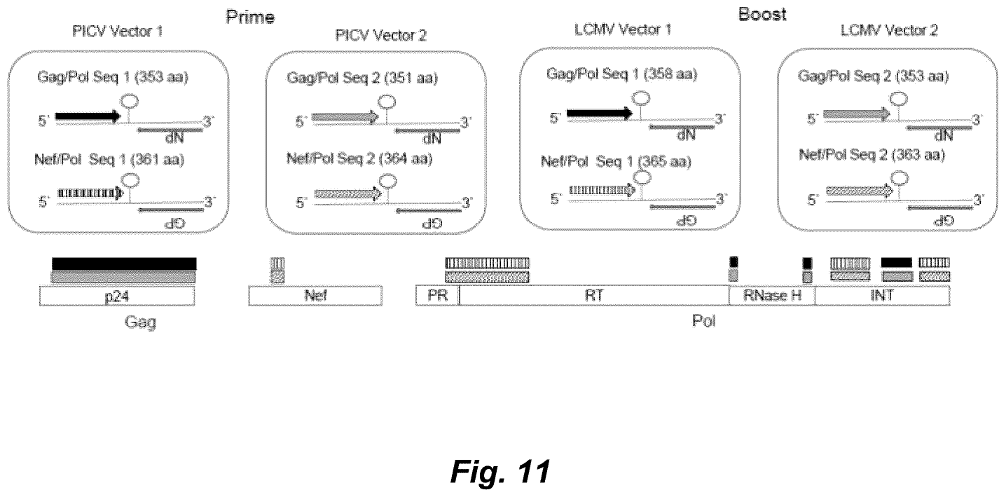

We utilized the protein coding regions of (1) Gag and Pol, (2) Pol and Nef to generate different bivalent constructs. In a final step, we applied our internally developed polypeptide segment arrangement tool described in Example 2 on the segments to reduce or eliminate possible presentation of deleterious or undesirable epitopes in junction regions. We anchored Nef conserved sequence to the C-terminus of the vaccine sequence during segment rearrangement for immunogen 2, because our previous experiments showed that positioning a Nef segment on the C-terminus of the vaccine sequences was more likely to induce immune responses. FIG. 11 provides a schematic of viral vectors containing the fusion proteins of immunogen version 2 (e.g., SEQ ID NOs: 82-89).

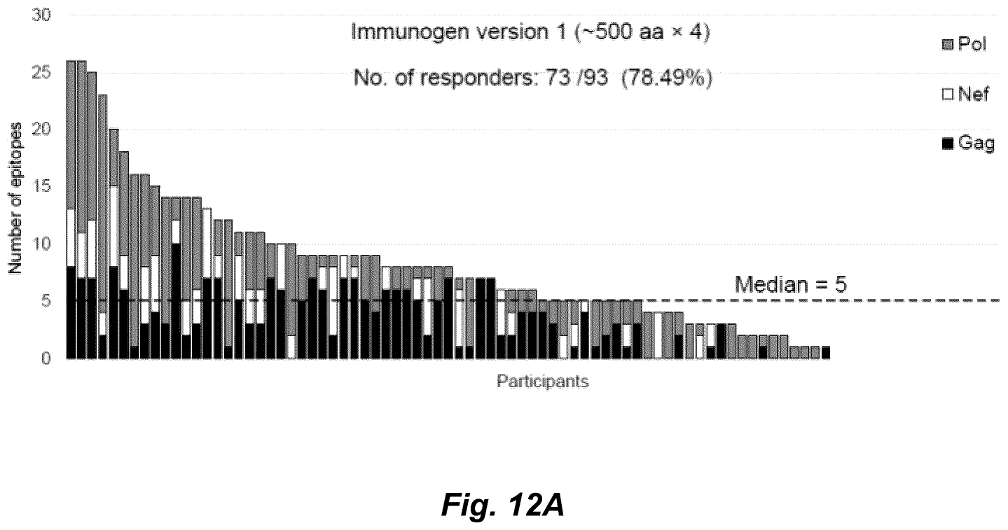

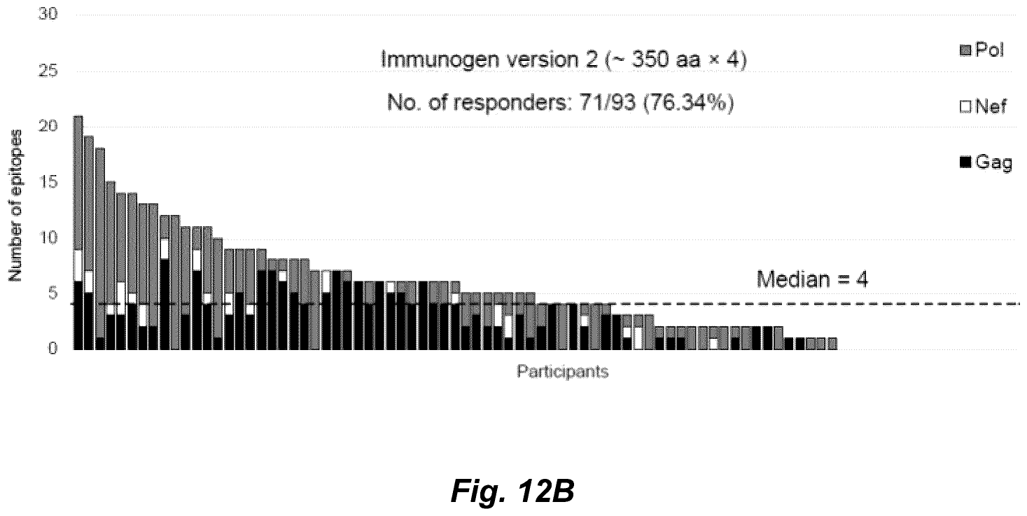

Median response and epitope density of the improved immunogen design. To assess whether the shortened immunogen is enriched with immunogenic regions, we compared the number of internally defined epitopes per sample between the fusion proteins designed according to the methods of Examples 1-2 (e.g., immunogen version 1; SEQ ID NOs: 94-101) and the fusion proteins designed according to the methods of Examples 1-3 (e.g., immunogen version 2; SEQ ID NOs: 82-89). FIGS. 12A-12B illustrate the median responses remain stable with size modification. Total of 93 samples were analyzed. We measured the median number of epitopes identified in each sample. The median number of epitopes of immunogen version 2 is 4 while the median number of epitopes of immunogen 1 is 5. Two non-responders were observed using shorter immunogen version 2. Epitope density was examined by assessing epitope rate. The epitope rate was defined as the number of in vitro clinical trial defined epitopes divided by the total potential epitopes in the immunogen. As illustrated in Table 1, the epitope rate analysis results demonstrate that we maximized the epitope density after size reduction of the fusion proteins of immunogen version 1 (516-527 amino acids in length) to the fusion proteins of immunogen version 2 (351-365 amino acids in length). The epitope rate of immunogen version 1 was maintained in immunogen version 2. We define the epitope rate as the percentage of all 9-mers that are recognized as epitopes by ELISpot assay (Total number of epitopes/the number of 9-mer positions).

TABLE 1
Epitope Rate Analysis Between Immunogen Versions 1 and 2

| | Immunogen version 1 | | | Immunogen version 2 | | |
|---|---|---|---|---|---|---|
| | Number of | Number of | | Number of | Number of | |
| | unique | 9mer | Epitope | unique | 9mer | Epitope |
| Gene | epitopes | positions | rate (%) | epitopes | positions | rate (%) |
| Gag | 64 | 259 | 24.71 | 53 | 215 | 24.65 |
| Nef | 13 | 50 | 26.00 | 2 | 5 | 40.00 |
| Pol | 107 | 572 | 18.71 | 84 | 419 | 20.04 |
| Total | 184 | 881 | 20.89 | 138 | 639 | 21.60 |

### Example 4

Shortened Construct Suitable for Expression from Single Priming Vector and Single Boosting Vector: Immunogen Version 3

In this Example, as in Steps 1-2 of Example 1 above, we first aligned the source sequences and then applied the CWA to identify a set of all candidate conserved regions in the protein coding regions of the target genes. In this example, the target genes were Gag, Nef and Pol. We applied the CWA to build bivalent sequences in those regions, as in Steps 3-5 of Example 1.

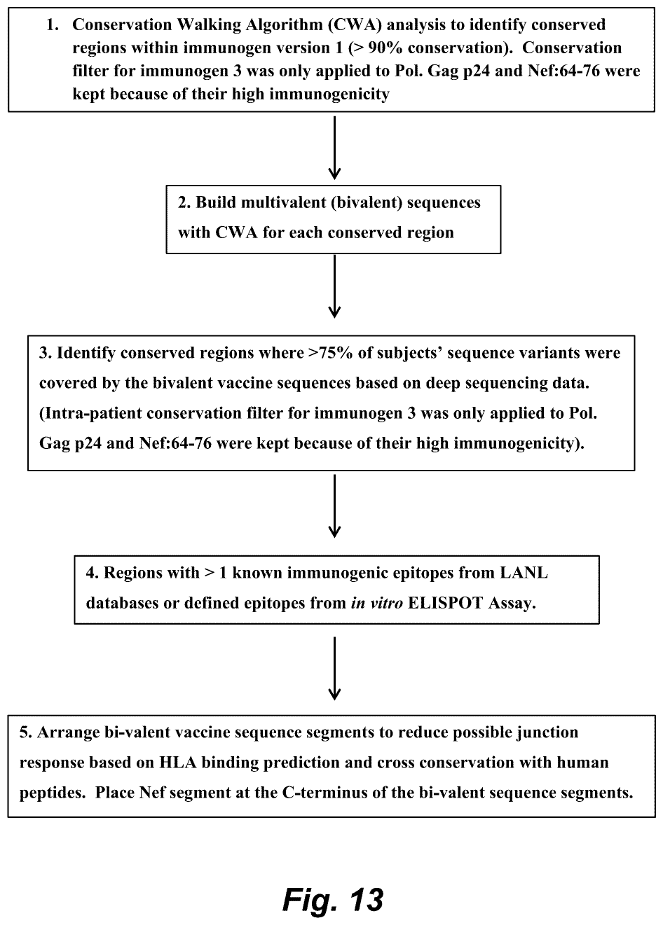

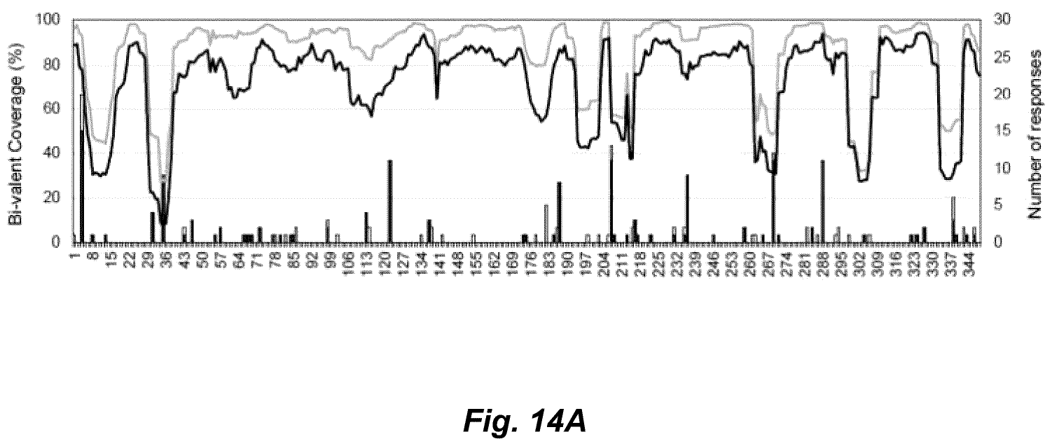

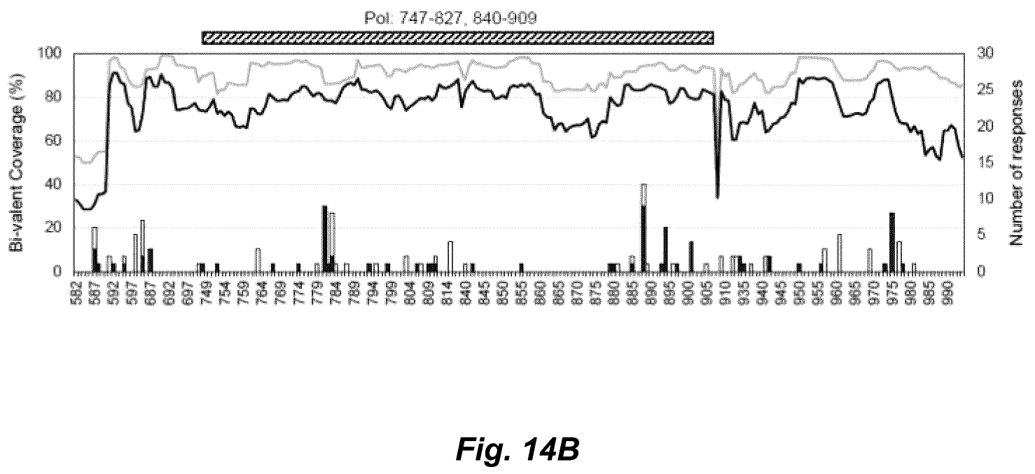

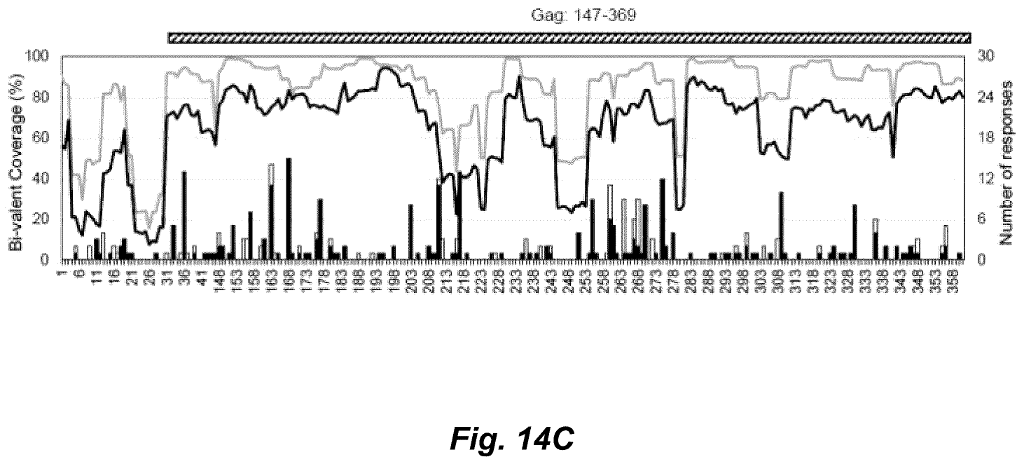

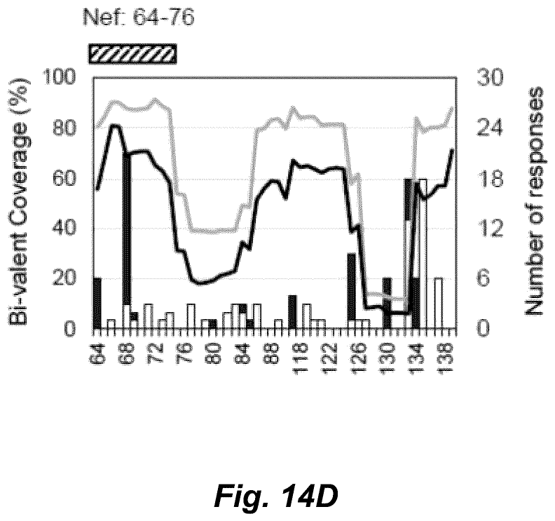

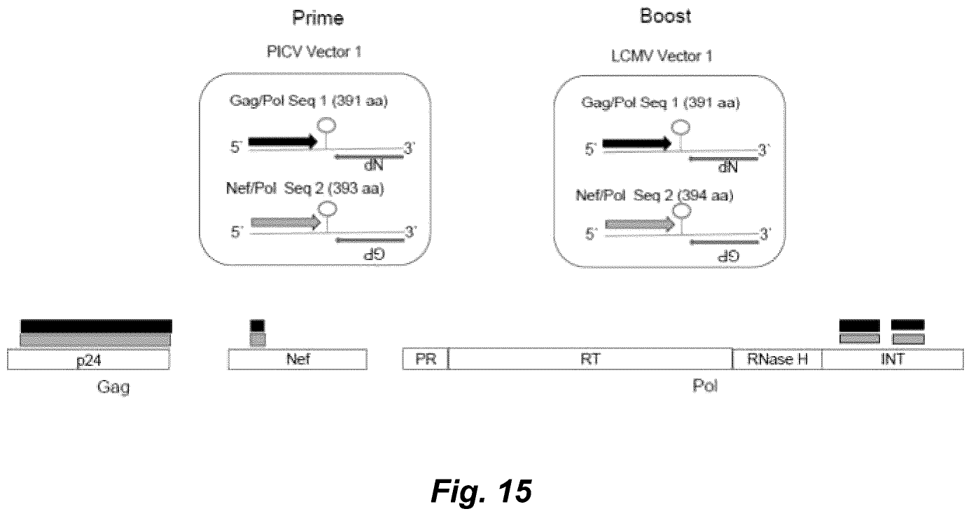

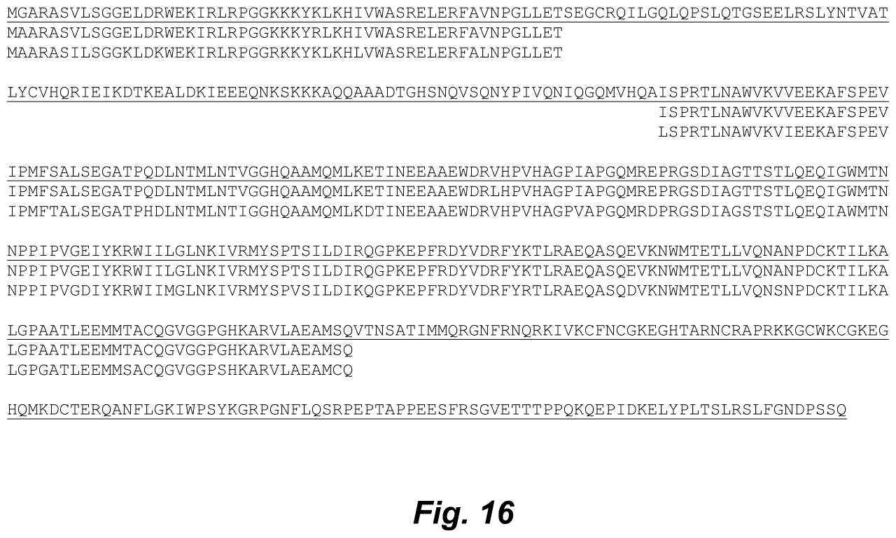

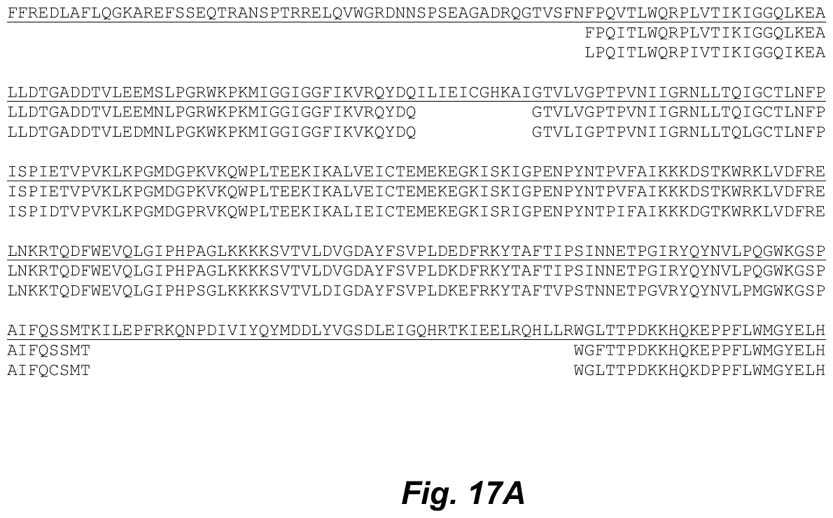

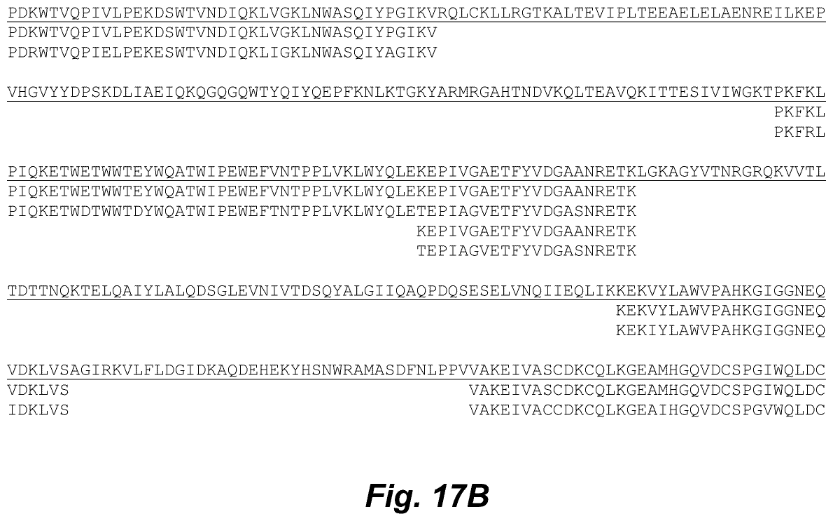

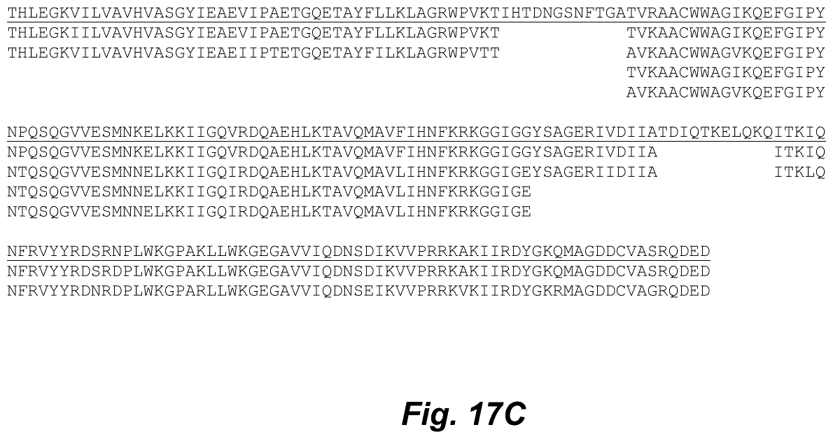

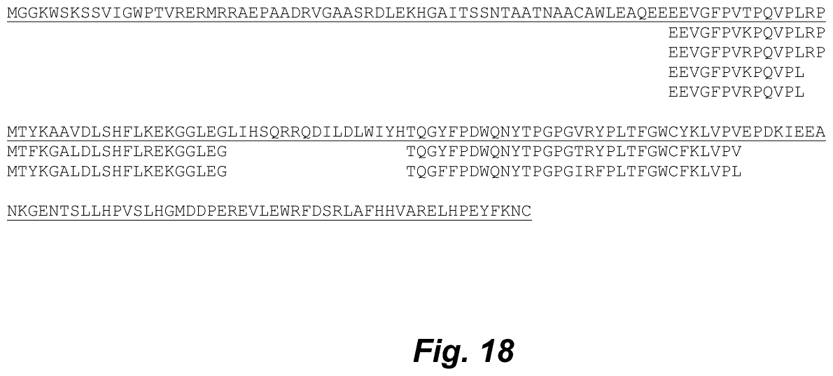

As Example 3 above, we applied deep sequencing and immunogenicity data to identify a subset of highly conserved and immunogenic regions within immunogen version 1 to retain in a shortened immunogen. FIG. 13 provides a flow diagram illustrating the basic methodology of the fusion polypeptides of immunogen version 3. SEQ ID NOs: 90-93 are illustrative fusion polypeptides designed according to this method. We utilized the same method described in Example 3 to design the shortened bivalent fusion polypeptides of immunogen version 3 (391-394 amino acids in length) of a length such that four fusion polypeptides could be expressed from two virus vectors (e.g., two arenavirus vectors). Immunogenicity data showed that Gag p24 and Nef segment (64-76) elicited strong immune responses, so Nef 64-76 was retained in Immunogen version 3. For Pol, as above, we identified the continuous regions in Pol that have intra-patient conservation >75% and contain multiple epitopes defined by in vitro assay. The FIGS. 14A-14D illustrate the regions selected for immunogen 3. We utilized the protein coding regions of (1) Gag and Pol, (2) Pol and Nef to generate different bivalent constructs. In a final step, we applied our internally developed polypeptide segment arrangement tool described in Example 2 on the segments to reduce or eliminate possible presentation of deleterious or undesirable epitopes in junction regions. A conserved subsequence of Nef was positioned at the C-terminus of the immunogen version 3 fusion polypeptides. FIG. 15 provides a schematic of viral vectors containing the fusion proteins of immunogen version 3 (e.g., SEQ ID NOs: 90-93).

### Example 5

### Viral Expression Vectors Containing Immunogenic Fusion Polypeptides in Immunogen 1

In this example, we generated viral expression vectors encoding the computationally defined polypeptide segments containing conserved regions of HIV-1 encoded by Gag, Nef and Pol genes as a transgene and confirmed expression of the transgene in mammalian cells. The polypeptide segments containing conserved regions were concatenated or connected by a variety of approaches including direct fusion, linkage of regions by the addition of a proteolytic cleavage site sequence or the addition of a flexible linker between regions

### Methods

Evaluation of target gene expression in vitro. To improve assembly of viral vectors encoding the vaccine expression cassette, the genes were cloned into vector plasmids (ThermoFisher Scientific) containing restriction sites for cloning target genes and a GFP marker. DNA was transformed into One Shot™ TOP10 competent cells (Invitrogen, Carlsbad, CA) following manufacturer's protocol and plated onto LB agar plate supplemented with 100 μg/ml ampicillin. The plate was incubated overnight at 37° C. A single colony was picked from the plate and inoculated into a 10 ml liquid LB+ampicillin culture and shaken overnight at 37° C. at 250 rpm in an Eppendorf bench top shaker. The bacterial pellet was processed using QIAprep Spin miniprep kit (Qiagen, Germantown, MD) to obtain the plasmid DNA following manufacturer's protocol. Nucleic acid concentration was determined by reading absorbance at 280 nm using NanoDropTM2000 (Thermo Scientific). To evaluate in vitro expression, the Adenovirus serotype 5 (Ad5) expression vectors expressing a compound fusion protein of SEQ ID NO: 105, 107, 109 or 111 were used to transduce monocyte derived dendritic cells (moDCs) as outlined in Example 9 below. At day 2 post-transduction, when the viability of cells was still at >80%, they were evaluated for GFP expression by flow cytometry or pelleted. The cell lysates were evaluated for HIV-1 Gag p24 expression by ELISA.

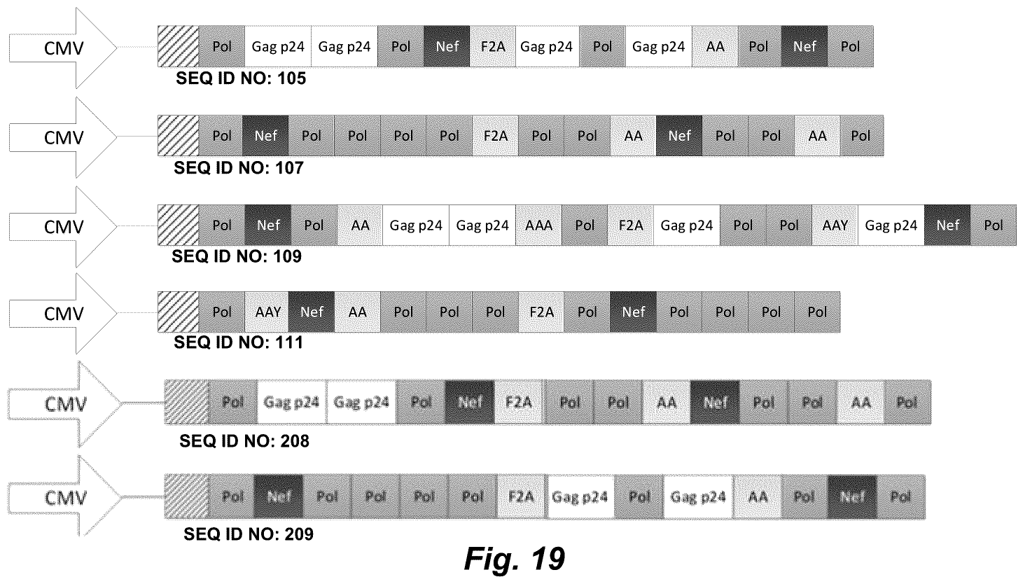

Construction of viral expression vector containing transgene encoding fusion polypeptide variants. Ad5 vectors expressing an HIV-1 computationally defined vaccine immunogen with various approaches to linkage of conserved HIV-1 sequences, were generated by in vitro recombination using standard methods (Vector Biolabs). Expression cassettes were generated by PCR using synthetic oligonucleotides codon-biased for improved human expression (IDT), and placed under the control of the CMV promoter using standard gene cloning techniques. The immunogen version 1 compound fusion polypeptide constructs developed for this evaluation are listed in Table F and schematically depicted in FIG. 19.

### Results

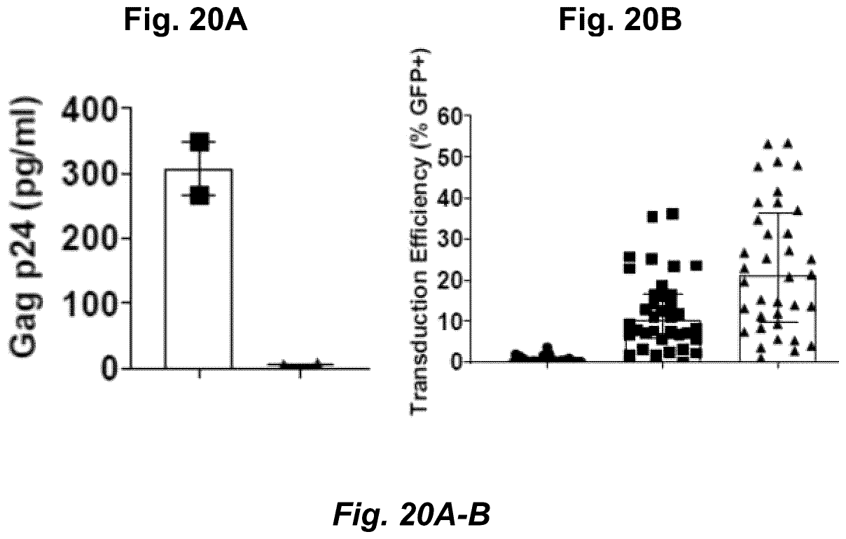

The data depicted in FIG. 20 demonstrate that all conserved region fusion polypeptide sequences were efficiently expressed (FIG. 20A) and transduced (FIG. 20B) as demonstrated by GFP expression and Gag p24 ELISA.

### Example 6

### Viral Expression Vectors Containing Immunogenic Fusion Polypeptides in Immunogen 2

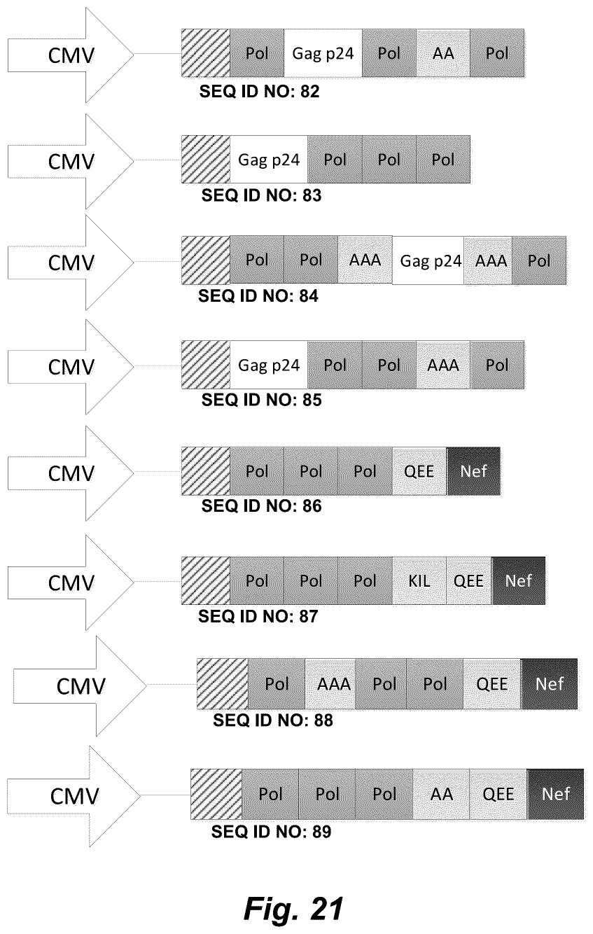

This example evaluates target gene expression in vitro and construction of Ad5 viral expression vectors. Similar strategy was adopted for evaluating the expression of conserved regions HIV Immunogen 2 (SEQ ID NO: 82-89) as described above in Example 5 for immunogen version 1 fusion polypeptides. The immunogen version 2 fusion polypeptide constructs are listed in Table E and schematically depicted in FIG. 21.

### Results

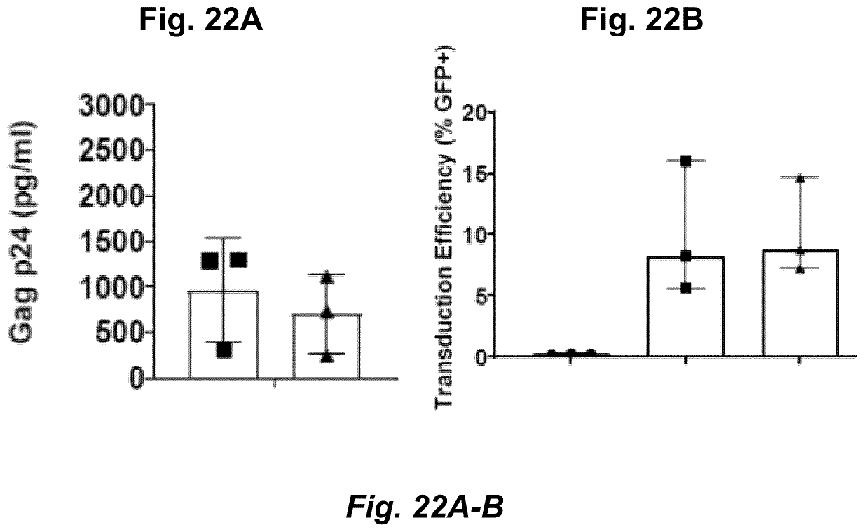

The data depicted in FIGS. 22A-22B demonstrate that all conserved region polypeptide sequences resulted in the efficient expression (FIG. 22A) and transduction (FIG. 22B) of polynucleotides encoding the fusion polypeptides of SEQ ID NOs: 82-89.

### Example 7

### Viral Expression Vectors Containing Immunogenic Fusion Polypeptides in Immunogen 3

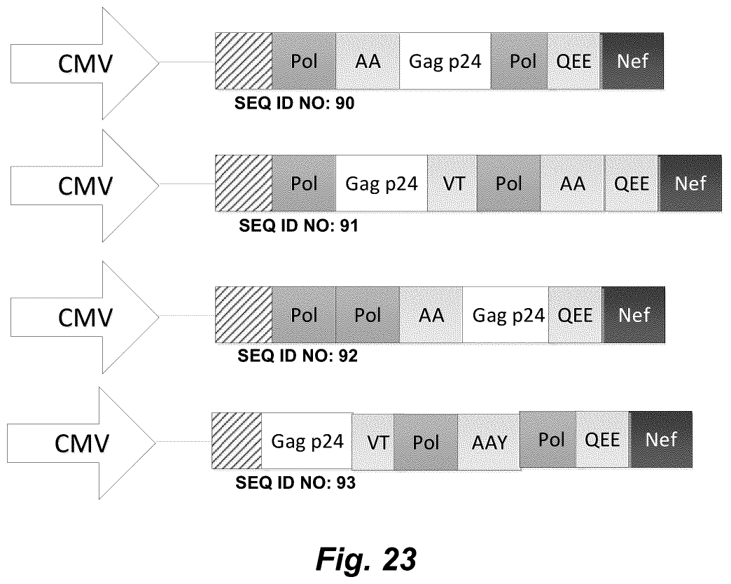

This example evaluates target gene expression in vitro and Construction of Ad5 viral expression vectors. Similar strategy was adopted for evaluating the expression of conserved regions HIV Immunogen 3 (SEQ ID NO: 90-93) as described above in Example 5 for the fusion polypeptides of immunogen version 1. The immunogen version 3 fusion polypeptide constructs are listed in Table E and schematically depicted in FIG. 23.

### Results

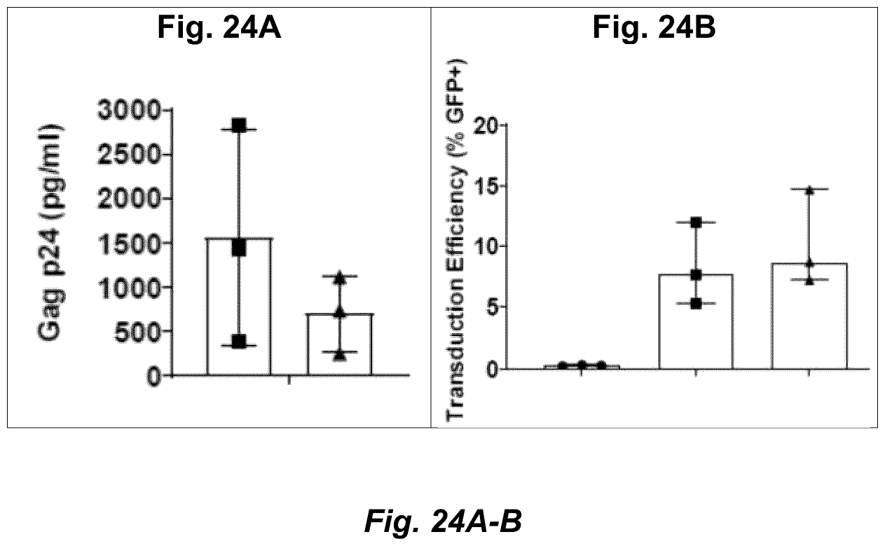

The data depicted in FIGS. 24A-24B demonstrate that all conserved region polypeptide sequences resulted in the efficient expression (FIG. 24A) and transduction (FIG. 24B) of polynucleotides encoding the fusion polypeptides of SEQ ID NOs: 90-93.

We then tested the efficiency of these constructs in various viral vector systems to prime T cell responses in vitro and in vivo.

### Example 8

### In Vivo T Cell Activation Assays

In this example, we evaluated the efficacy of in vivo T cell priming by vaccine constructs in a mouse model. To do this, we immunized groups of mice with Ad5 vectors expressing computationally defined conserved regions vaccine immunogen sequences. We investigated the magnitude and functional phenotype of those responses to determine the immunogenicity of the vaccine sequences.

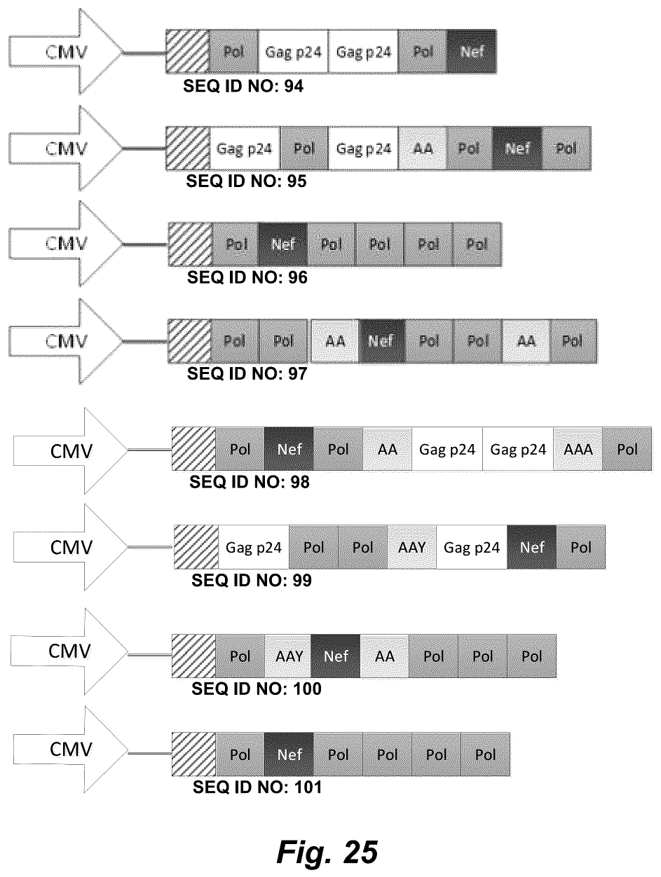

The vaccines were administered in a homologous (with respect to viral expression vector) prime-boost approach. Each viral Ad5 vector in the prime-boost pair had different rearrangements of the HIV gene segments encoded in the fusion polypeptide construct. This gene rearrangement reduced or eliminated the formation of junctional responses. Vectors may differ in the size of transgene that they can stably express. In the design of the vaccine immunogens two complementary fusion polypeptides of less than 600 amino acids in length each were expressed from a single viral vector. Vectors that can accommodate larger transgenes may combine two segments, e.g., using an F2A linker (described herein as “compound fusion polypeptides;” see Table F). We tested the immunogenicity of each individual fusion polypeptide sequence in a priming sequence, and again in a prime-boost sequence using vectors expressing compound fusion polypeptides with and without the F2A linker. Vectors that contained F2A linker also had an N-terminal tissue plasminogen (t-PA) signal peptide sequence (MDAMKRGLCCVLLLCGAVFVSAR (SEQ ID NO: 121)). The immunogen version 1 fusion polypeptide constructs are listed in Table E and schematically depicted in FIG. 25. The immunogen version 1 compound fusion polypeptide constructs are listed in Table F and schematically depicted in FIG. 19.

### Methods

### In Vivo Evaluation of Immunogenicity

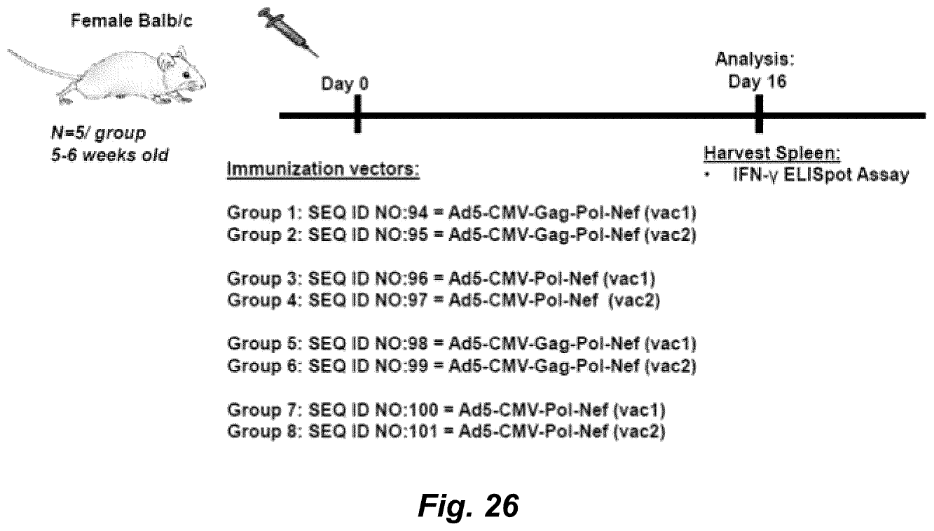

Immunizations. Five or sᵢₓ-week-old Balb/c mice were immunized with 1×10⁹ PFU of Ad5 vectors expressing HIV immunogens by intramuscular (I.M.) injections in both hind leg muscles. The vaccine vector was administered in 100 μl of phosphate-buffered saline (PBS) injections (50 μl per quadriceps). Mice were anesthetized with isoflurane prior to vaccine immunization. Animals were housed at the animal facility (Bioqual, Maryland) and experiments were performed according to approved IACUC protocol. A schematic of the regimen is provided in FIG. 26.

Single vector immunogenicity. Mice were randomly assigned into 8 groups and were primed with 1×10⁹ PFU of Ad5 vectors expressing HIV immunogens (SEQ ID NOs: 94-101, schematically depicted in FIG. 25) by intramuscular (I.M.) injections in both hind leg muscles and rested for 16 days before splenocytes were harvested. Immunogenicity was evaluated by analyzing splenocytes by IFN-γ ELISpot assay as previously described (Miyahira, et al., J Immunol Methods, (1995) 181 (1): 45-54).

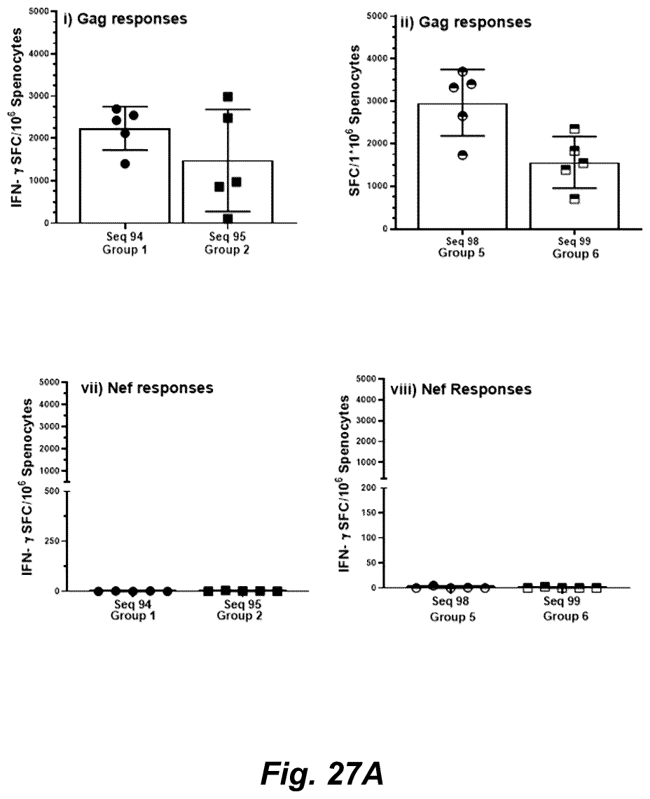

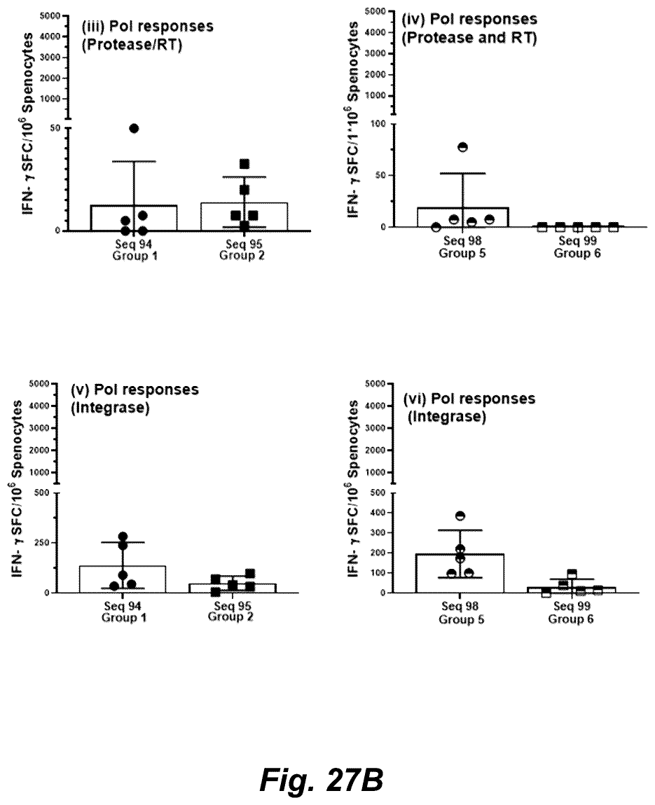

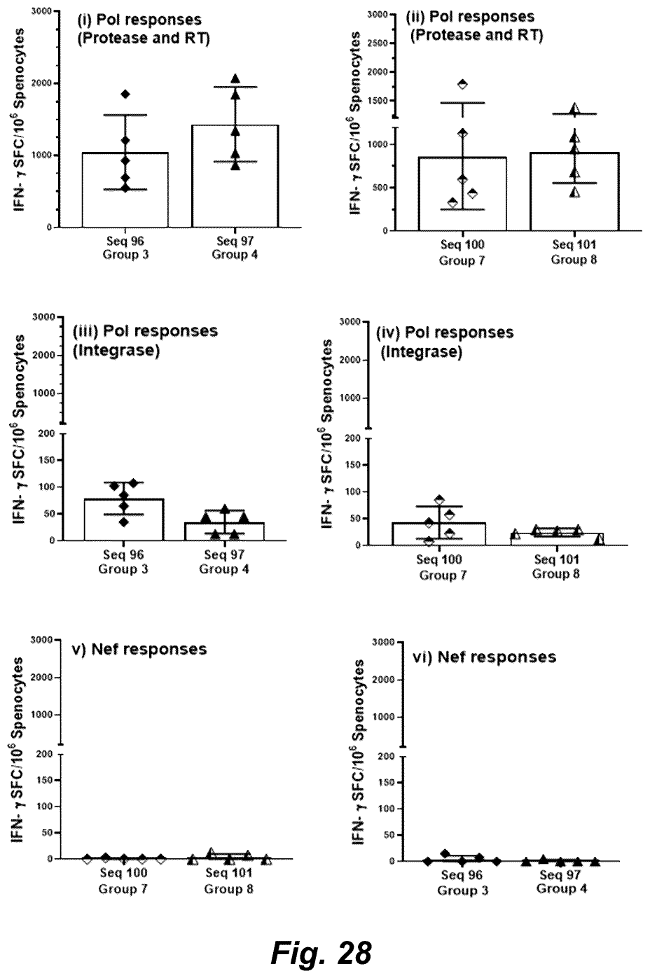

ELISpot Assays. Pre-coated strip ELISpot plates (Cellular Technologies Limited) were used for all ELISpot analyses. Briefly, 2×105 splenocytes from immunized animals were seeded to each well. Peptide pools having 15-mer peptides overlapping by 11 amino acid residues spanning the HIV Gag, Pol and Nef conserved regions sequences were used in IFN-γ ELISpot assays to evaluate vaccine immunogenicity. 2.5 ng/ml PMA and 250 ng/ml ionomycion as well as lug/mL ConA were used as positive controls. Plates were incubated at 37° C. in 5% CO2 overnight and up to 18 hrs. After overnight stimulation, the cells were removed from the plates and the wells were washed two times in PBS prior to two washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming units (SFUs) were counted on an Immunospot ELISpot reader. The settings were identical for all plates and counts were expressed at SFU per 106 splenocytes. A schematic of the vector variants is provided in FIG. 25, the immunization regimen is provided in FIG. 26, and the results are shown in FIGS. 27-28.

Homologous prime-boost. Mice were randomly allocated into 6 groups. Groups 1 and 2 mice were immunized with 1×10⁹ PFU of Ad5 vectors expressing bivalent fusion polypeptides of SEQ ID NO: 105 (Seq 94-F2A-95) and SEQ ID NO: 109 (Seq 98-F2A-99) having N-terminal t-PA leader sequences, respectively, by intramuscular (i.m.) injections in both hind leg muscles and rested for 16 days before splenocytes were harvested. Groups 3 and 4 mice were immunized with 1×10⁹ PFU of Ad5 vectors expressing bivalent fusion polypeptides of SEQ ID NO: 107 (Seq 96-F2A-97) and SEQ ID NO: 111 (Seq 100-F2A-101) having N-terminal t-PA leader sequences, respectively, by i.m. injections in both hind leg muscles and rested for 16 days before splenocytes were harvested.

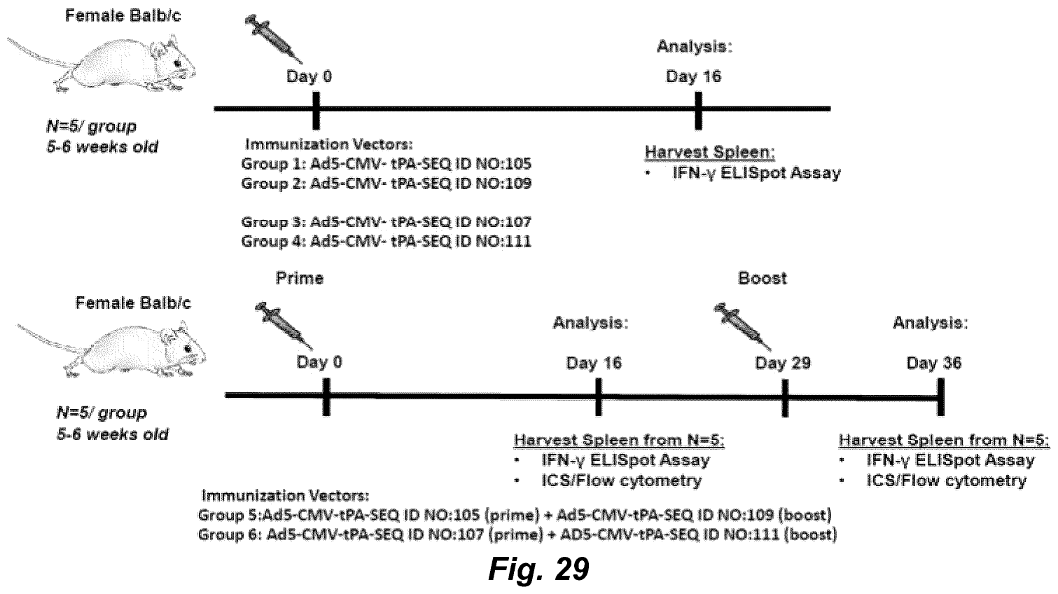

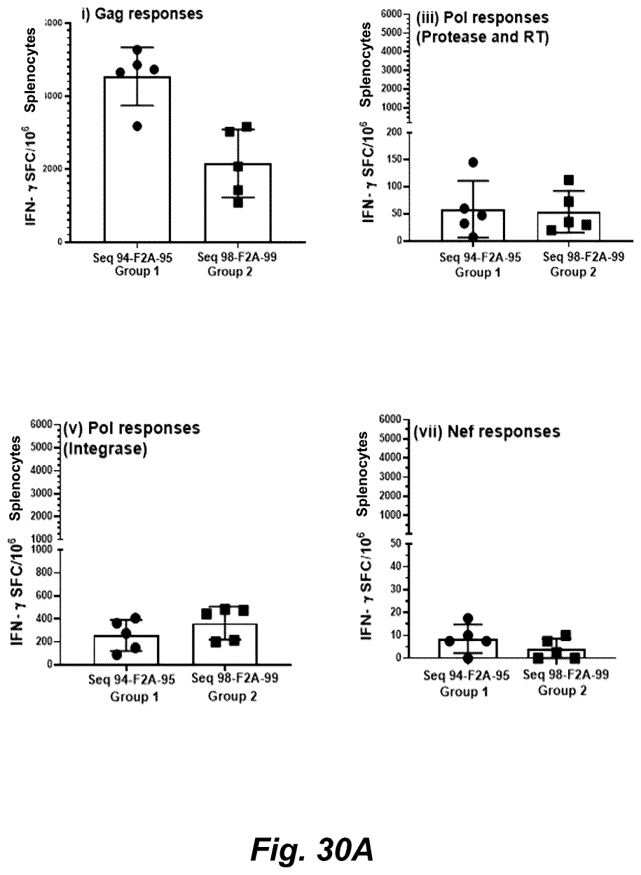

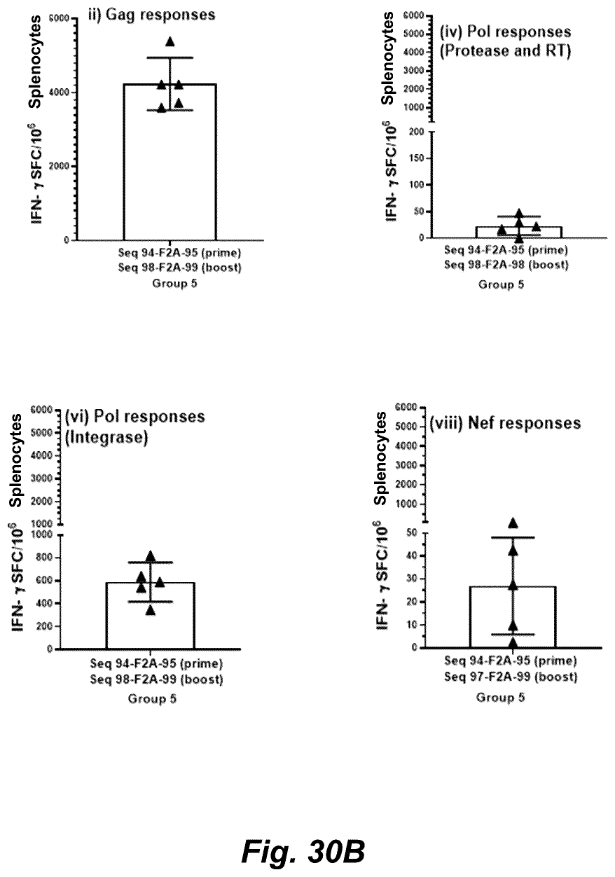

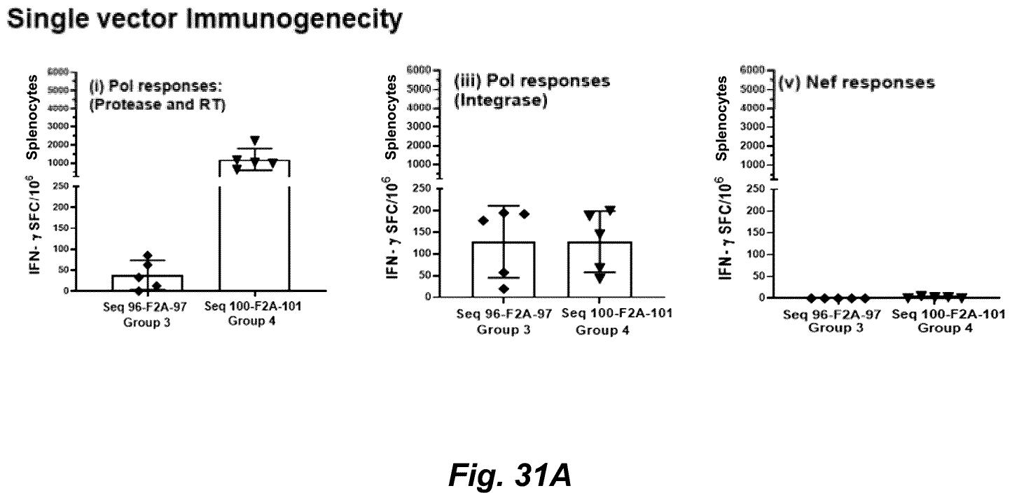

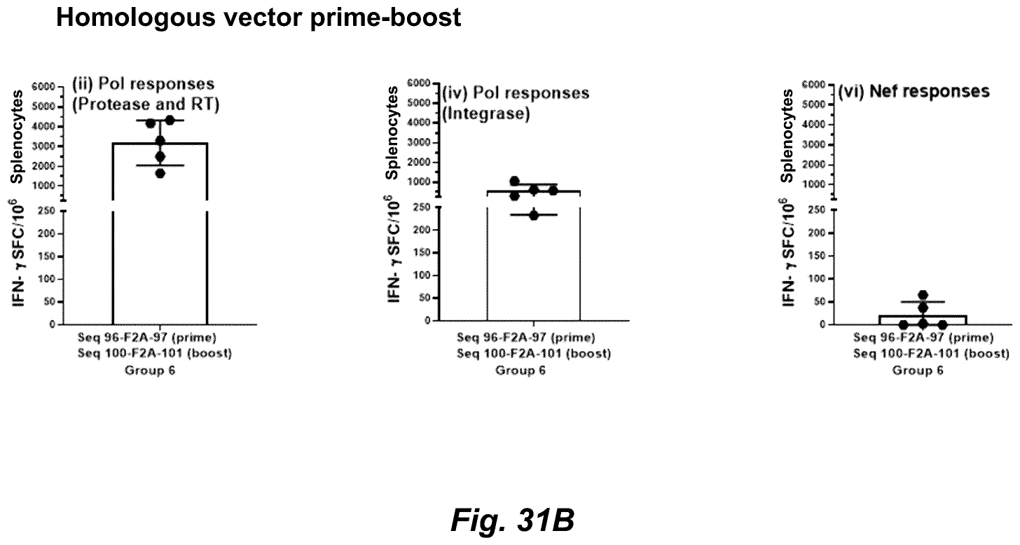

Group 5 and 6 mice were immunized with 1×10⁹ PFU of Ad5 vectors Seq-94-F2A-95 (tPA-SEQ ID NO: 105) and Seq 96-F2A-97 (tPA-SEQ ID NO: 107) expressing HIV immunogens utilized in Group 1 or 3 respectively, by i.m. injections in both hind leg muscles and rested for 29 days before homologous boost with vectors expressing the same antigens but rearranged to minimize junctional responses (Seq 98-F2A-99 (tPA-SEQ ID NO:10⁹) and Seq 100-F2A-101 (tPA-SEQ ID NO: 111) immunogens utilized in Group 2 and 4 respectively). Immunogenicity and cellular phenotype were evaluated by analyzing splenocytes on Day 36 by ELISpot assay as previously described (Miyahira, et al., J Immunol Methods, (1995) 181 (1): 45-54), ICS by flow cytometry was conducted at day 16 after prime and day 36 after prime/boost. A schematic of the vector variants is provided in FIG. 19, the immunization regimen provided in FIG. 29 and results are shown in FIGS. 30-31.

Flow cytometry. Cell counts for prepared single-cell suspensions were determined using a hemacytometer. 1×10⁶ cells/condition were stimulated with relevant HIV peptides in the presence of Golgi plug for a total of 6 hr (no more than 18 hrs). Washed cells were surface stained with live dead Aqua (Thermo fisher L34957) dye first and incubated with a mixture of fluorescence-conjugated anti-mouse antibodies for 20 min at 4° C. CD3 APC-Cy7 clone 17A2, CD4 PE-Cy7 clone GK 1.5, CD8 percp-Cy5.5 clone 53-6.7 were used for surface staining. After surface staining, cells were fixed and permeabilized in preparation for intracellular cytokine staining. Briefly, 1×10⁶ cells already stained with surface antibodies were incubated with 200 μl BD cytofix/cytoperm buffer for 25 minutes on ice. Subsequently, cells were washed twice with 200 μl 1XPerm buffer each time and were then incubated with a cocktail of antibodies diluted in 100 μl of Perm buffer per 1×10⁶ cells. A cocktail of fluorophore-conjugated anti mouse anti-IFN-γ APC clone XMG1.2, anti-IL-2 PE clone JES6-5H4 and anti-TNF-α FITC clone MP6-XT22 were used for intracellular cytokine staining. Permeabilized cells were then washed with 100 μl Perm buffer twice and immediately analyzed on MACSQUANT 10 Analyzer using MACS Quantify version 2.13 (Miltenyi Biotech) and analyzed using FlowJo software version 10.7 (TreeStar).

### Results

The various viral Ad5 vector constructs expressing immunogen version 1 fusion polypeptides (SEQ ID NOs: 94-101; depicted in FIG. 25) were effective in priming a T cell response. Immunogenicity of these sequences was studied in a Balb/C mouse model by IM immunization as shown in FIG. 26.

Priming bivalent fusion polypeptides SEQ ID NOs: 94-95 and boosting bivalent fusion polypeptides SEQ ID NOs: 98-99 having HIV-1 Gag, Pol and Nef sequence segments induced robust responses to HIV-1 Gag (FIG. 27A (i and ii), with weaker but detectable responses to Pol proteins (protease, RT and integrase, (FIG. 27A (iii to vi). No responses were detected to HIV-Nef (FIG. 27A (vii and viii) in this model likely due to previously described immunodominance patterns for HIV-1 Gag epitopes in Balb/c mice and reflecting the lack of Nef epitopes that can be presented in Balb/c mice.

Priming bivalent fusion polypeptides SEQ ID NOs: 96-97 and boosting bivalent fusion polypeptides SEQ ID NOs: 100-101 having HIV-1 Pol and Nef induced robust responses to HIV-1 Pol, especially protease and RT (FIG. 28 (i and ii), with weaker responses to Pol integrase (FIG. 28 (iii and iv). Responses to HIV-Nef (FIG. 28 (v and vi) were also not seen with this set of sequences corroborating the observations from previous set of sequences (FIG. 27A (vii and viii)).

To test if combining these bivalent construct sequences designed to ensure coverage of >80% of circulating HIV-1 viral sequences into a single construct affects immunogenicity, individual sequences were combined using an F2A linker as shown in FIG. 19 (SEQ ID NOs: 105, 107, 109 and 111). The compound fusion polypeptides each had an N-terminal tPA leader sequence. Bivalent fusion polypeptide pairs, SEQ ID NOs: 94 and 95 were combined into Seq 94-F2A-95 (tPA-SEQ ID NO: 105), SEQ ID NOs: 96 and 97 were combined into Seq 96-F2A-Seq 97 (tPA-SEQ ID NO: 107), SEQ ID NOs: 98 and 99 were combined into Seq 98-F2A-Seq 99 (tPA-SEQ ID NO: 109) and SEQ ID NOs: 100 and 101 were combined into Seq 100-F2A-Seq 101 (tPA-SEQ ID NO: 111), respectively.

To test immunogenicity, these combined sequences were tested by IM immunization in Balb/C mice (FIG. 29), as single vectors in a prime only mode (FIG. 30A and in a homologous prime boost combination mode (FIG. 30B). Sequences Seq 94-F2A-95 (tPA-SEQ ID NO: 105) and Seq 98-F2A-99 (tPA-SEQ ID NO: 107) were used as bivalent fusion polypeptides having HIV-1 Gag, Pol and Nef sequence segments and were tested as prime and boost sequences in homologous vector prime boost combination mode. Seq 96-F2A-97 (tPA-SEQ ID NO: 109) and Seq 100-F2A-Seq 101 (tPA-SEQ ID NO: 111) were used as bivalent fusion polypeptides having HIV-1 Pol and Nef and were tested as prime and boost sequences in homologous vector prime-boost combination mode. The priming and boosting fusion polypeptides encode similar regions but are rearranged in order to reduce or eliminate creation of de novo epitopes that resemble epitopes from the human proteome and to reduce or eliminate boosting of junctional responses in prime boost sequences.

Combined sequences Seq 94-F2A-95 (tPA-SEQ ID NO: 105) and Seq 98-F2A-99 (tPA-SEQ ID NO: 109) when tested for immunogenicity either as single prime or in a prime/boost combination were immunogenic (FIG. 31A, responses to HIV-Gag (i), Pol (protease and RT (iii), Pol (integrase (v)) and Nef (vii) respectively and FIG. 27 HIV-1 Gag (i and ii), Pol (protease, RT and integrase (iii to vi) and Nef (vii and viii). Bivalent sequences concatenated with F2A did not inhibit their immunogenicity. Overall, responses to HIV-1 Gag were robust with weaker but detectable responses to Pol proteins (protease, RT and integrase) and minimal responses to HIV-Nef. Responses induced by priming sequence Seq 94-F2A-95 (tPA-SEQ ID NO: 105) were enhanced by boosting sequence Seq 98-F2A-99 (tPA-SEQ ID NO: 10⁹), especially for Pol (integrase) (FIG. 30B (vi) and Nef (FIG. 30B (viii). No responses were generated to the F2A and tPA sequences.

Combined sequences Seq 96-F2A-97 (tPA-SEQ ID NO: 107) and Seq 100-F2A-101 (tPA-SEQ ID NO: 111) when tested for immunogenicity either as single prime or in a prime/boost combination were immunogenic, inducing robust responses to HIV-1 Pol, particularly protease and RT, with weaker responses detected toward integrase (FIG. 31A (i to iv). Magnitude of IFN-γ ELISpot responses induced by the combination vector Seq 96-F2A-97 (tPA-SEQ ID NO: 107) to Pol (protease and RT, FIG. 31B (i) were weaker than responses previously observed individual vectors expressing a fusion polypeptide of SEQ ID NO: 96 or 97 (FIG. 28 (i). The responses were boosted in sequential dosing with Seq 96-F2A-97 (tPA-SEQ ID NO: 107) and Seq 100-F2A-101 (tPA-SEQ ID NO: 111) (FIG. 31B (ii). The responses induced by priming sequence Seq 96-F2A-97 (tPA-SEQ ID NO: 107) were enhanced by boosting sequence Seq 100-F2A-101 (tPA-SEQ ID NO: 111) against both Pol (integrase) (FIG. 31B (iv) and Nef (FIG. 31B (vi)).

The ability to produce cytokines is a functional measure of effector and memory CD4+ and CD8+T cells. We evaluated the phenotypic and functional characteristics of CD4+ and CD8+ T cell responses generated following immunization with Ad5 vectors expressing compound fusion proteins of SEQ ID NOs: 105, 107, 109 or 111, using both single vector prime immunization and homologous prime/boost immunization. Responses were measured using ICS and Flow cytometry.

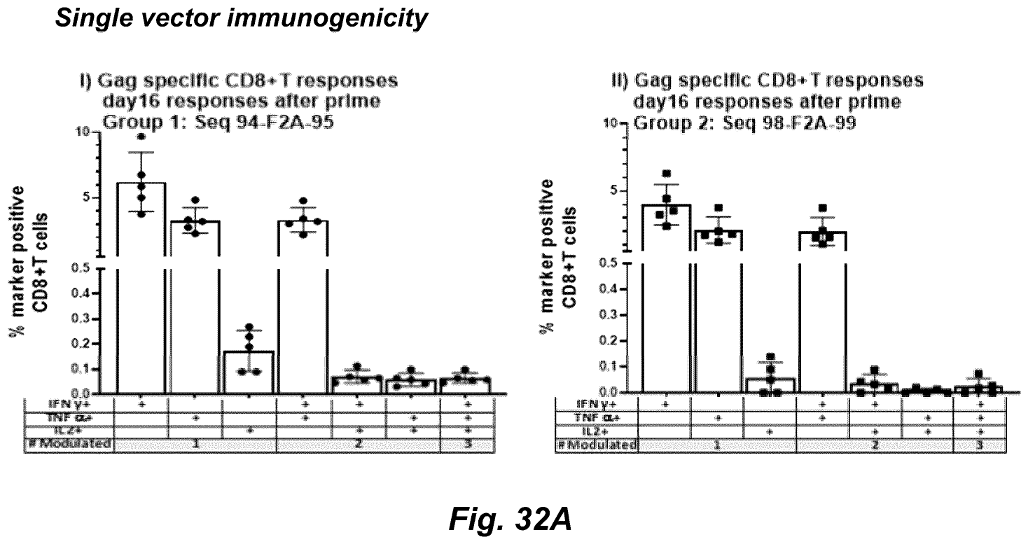

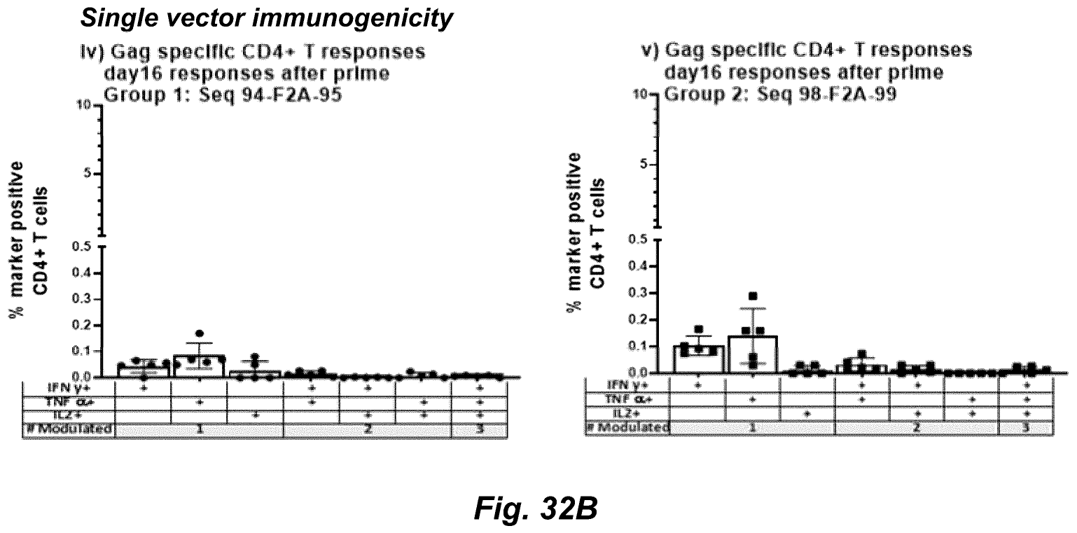

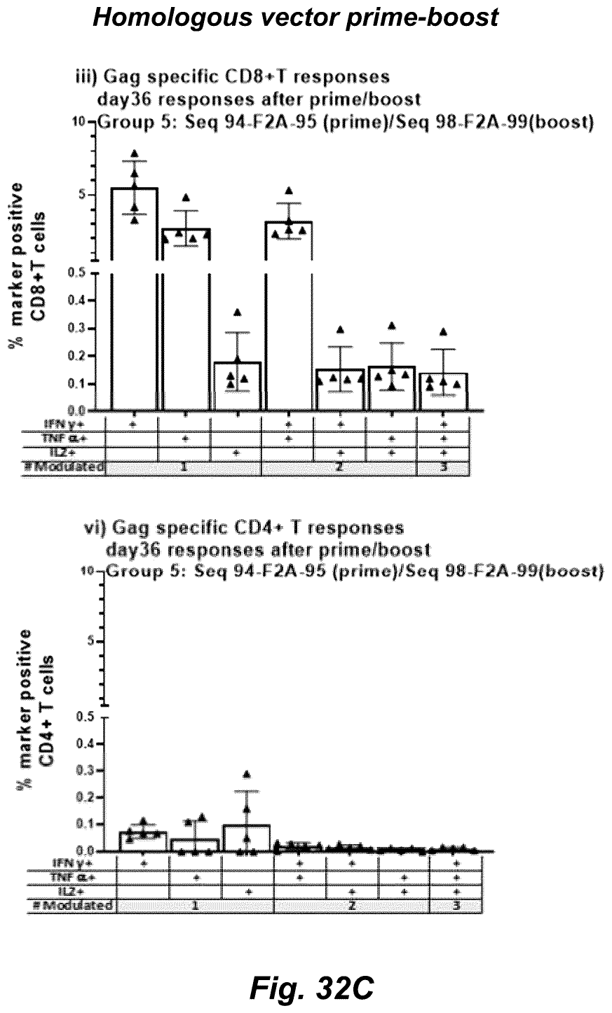

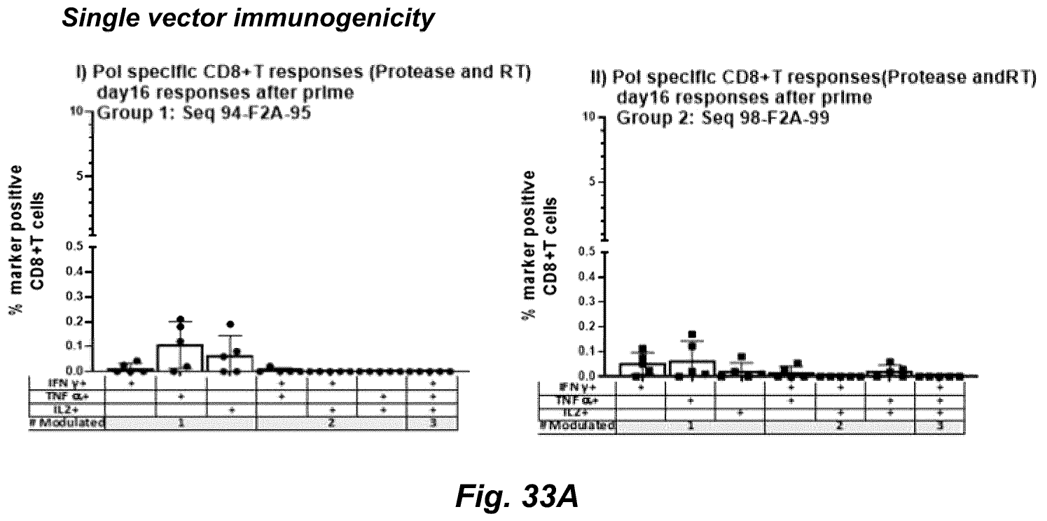

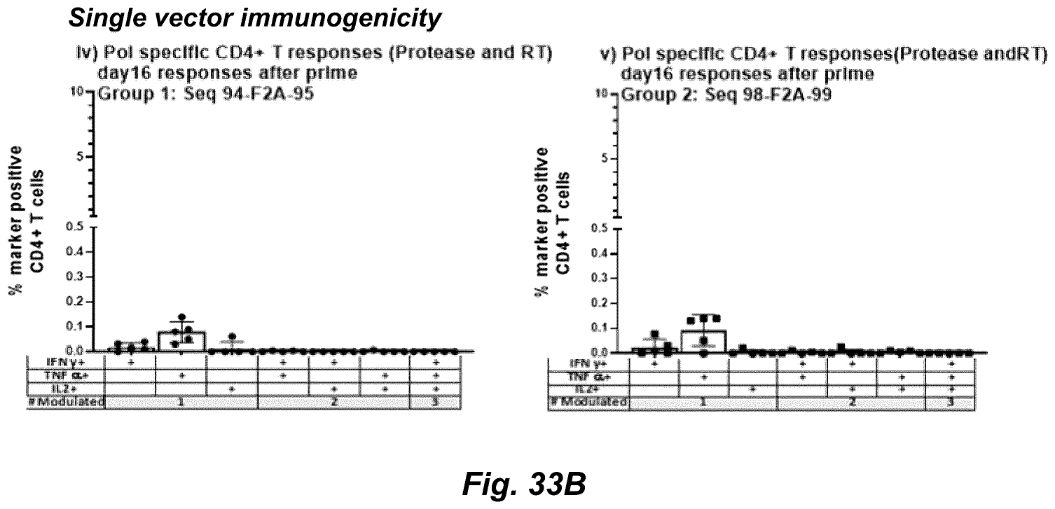

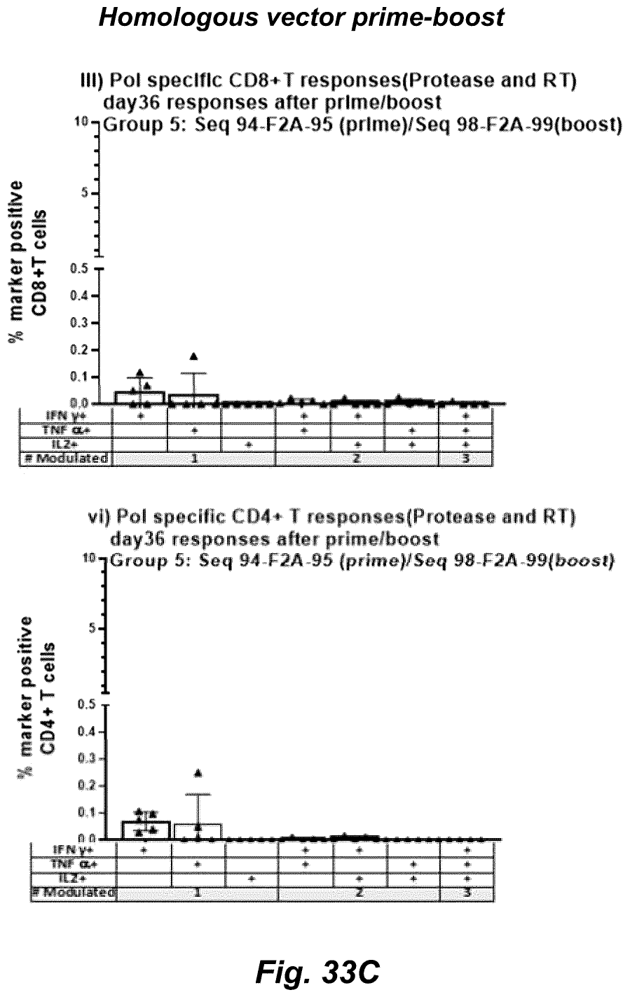

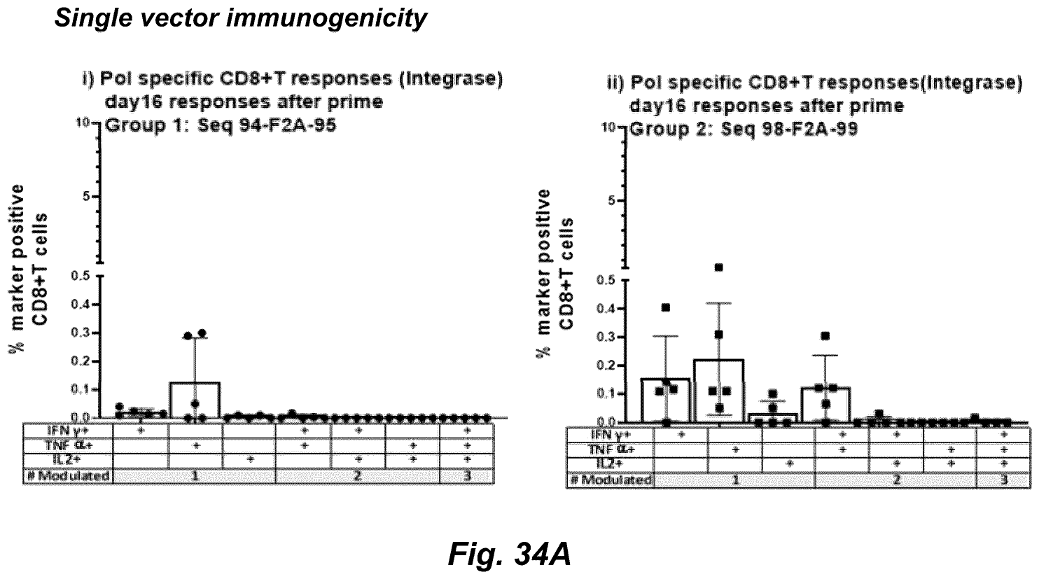

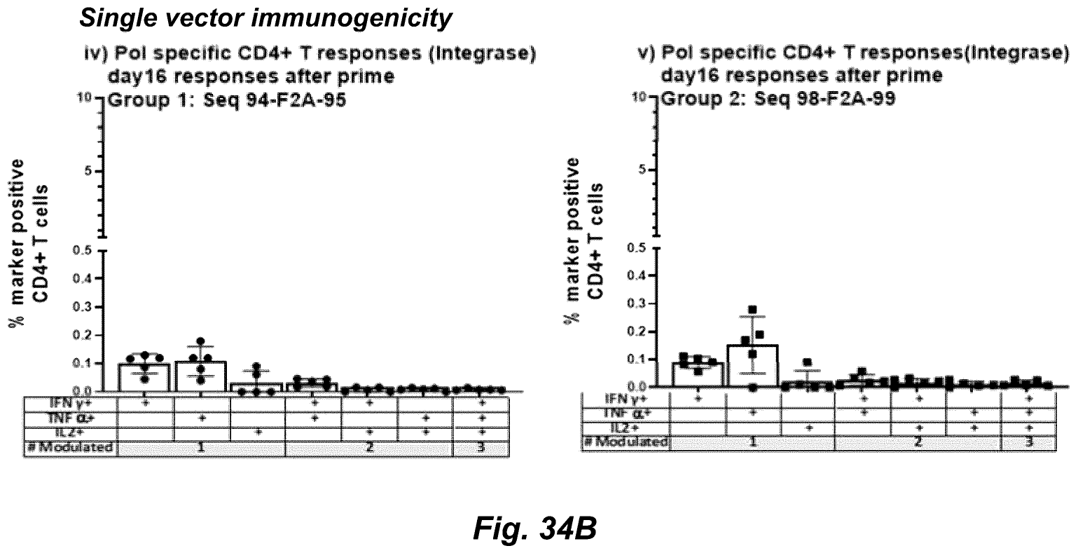

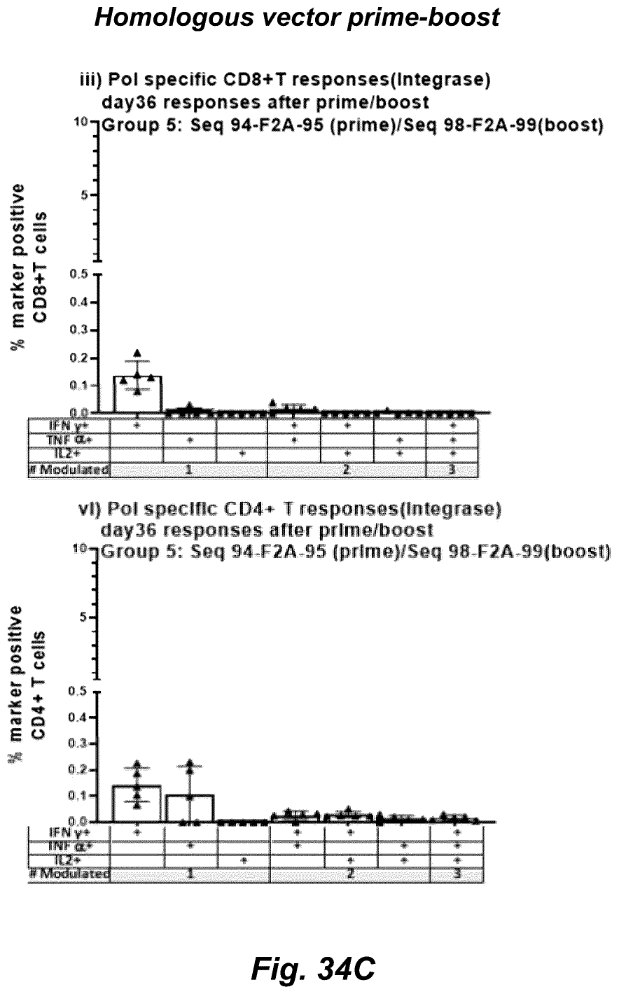

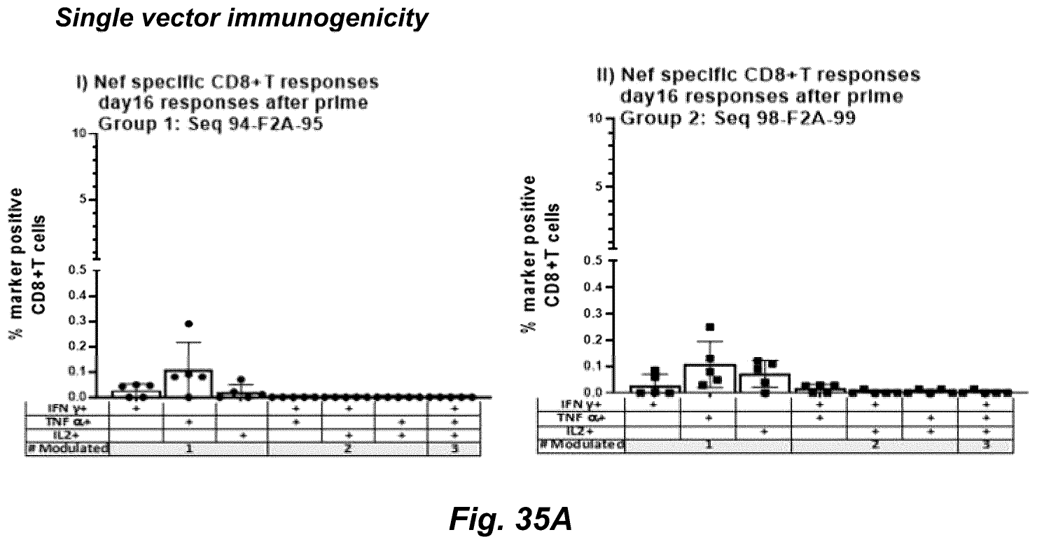

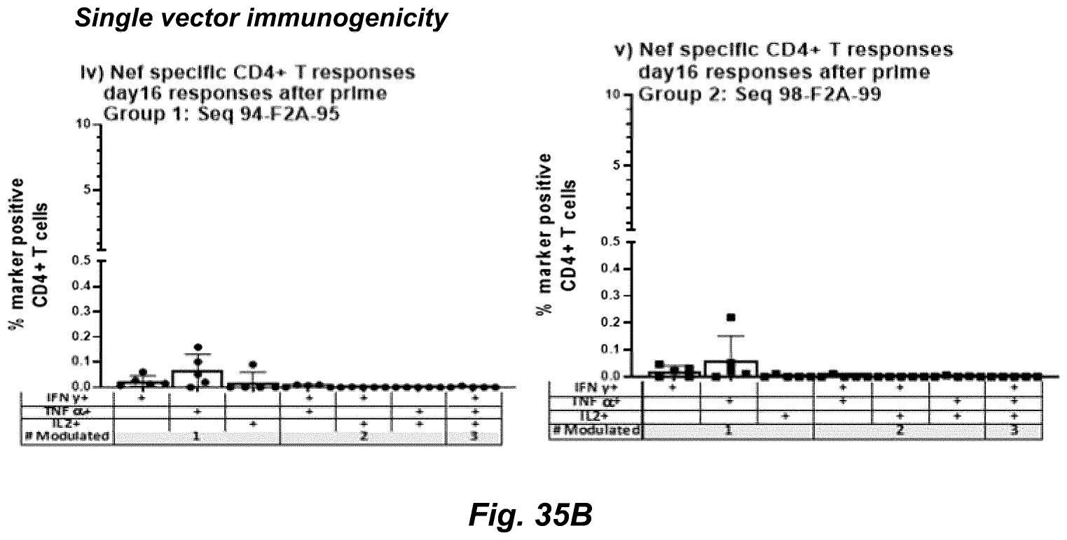

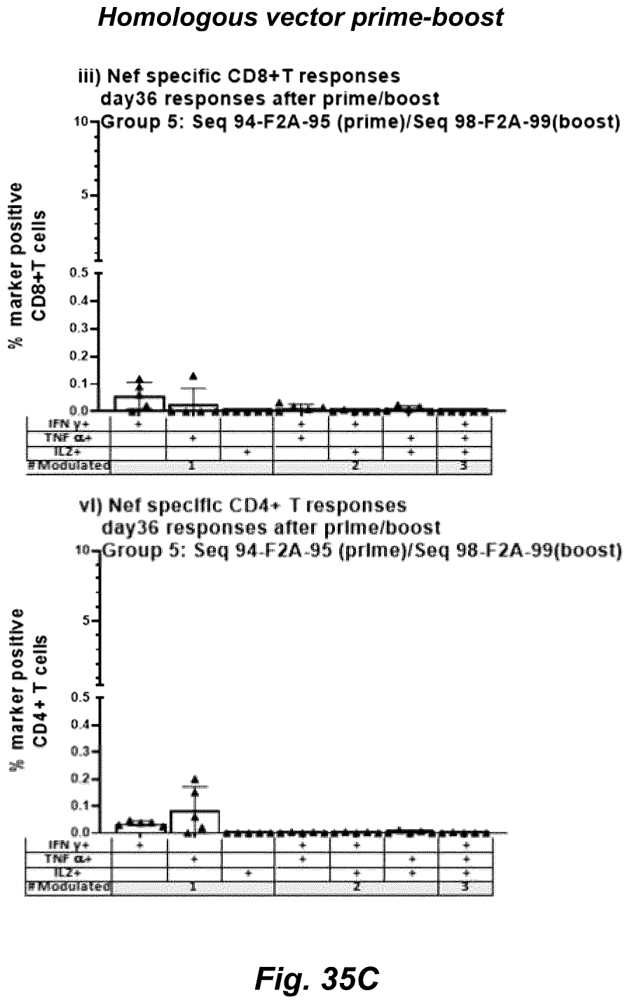

FIGS. 32-35 show immunogenicity against HIV-1 Gag, Pol and Nef antigens by intracellular cytokine staining (ICS) following single vector immunization with Seq 94-F2A-95 (tPA-SEQ ID NO: 105), or Seq 98-F2A-99 (tPA-SEQ ID NO: 109), and by homologous vector prime-boost immunization. The Y axis represents proportion of CD8+ (i, ii and iii) or CD4+ (iv, v and vi) T cells exhibiting HIV-1 Gag (FIG. 32), Pol (protease and RT) (FIG. 33), Pol (integrase (FIG. 34) and Nef (FIG. 35) specific responses by expressing cytokines IFN-γ, IL-2 and TNF-α, either as individual cytokines or combination of cytokines, as shown on X axis. Antigen specific values were obtained by subtracting no antigen stimulated control to exclude nonspecific responses. Strong HIV-1 Gag specific CD8+ and CD4+ T cell responses were seen in response to vaccination, with demonstrated polyfunctionality most robust in CD8+T cells. Homologous prime-boost enhanced polyfunctionality of the CD8+ T cell responses. Even though generated, the Pol (protease, RT and integrase) specific and Nef specific CD8+ and CD4+ T cell responses produced after vaccination were weaker than Gag responses and tend to be monofunctional, with limited boosting.

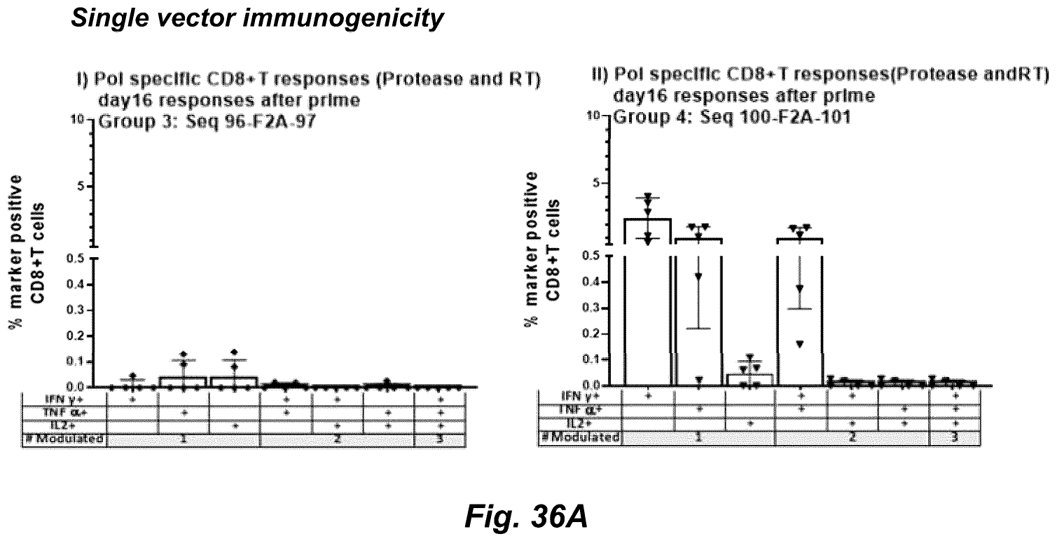

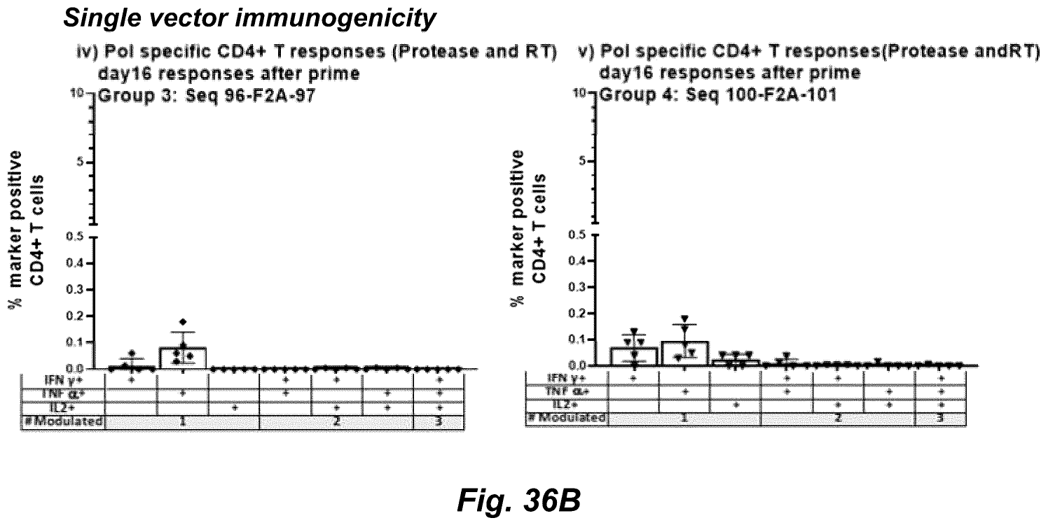

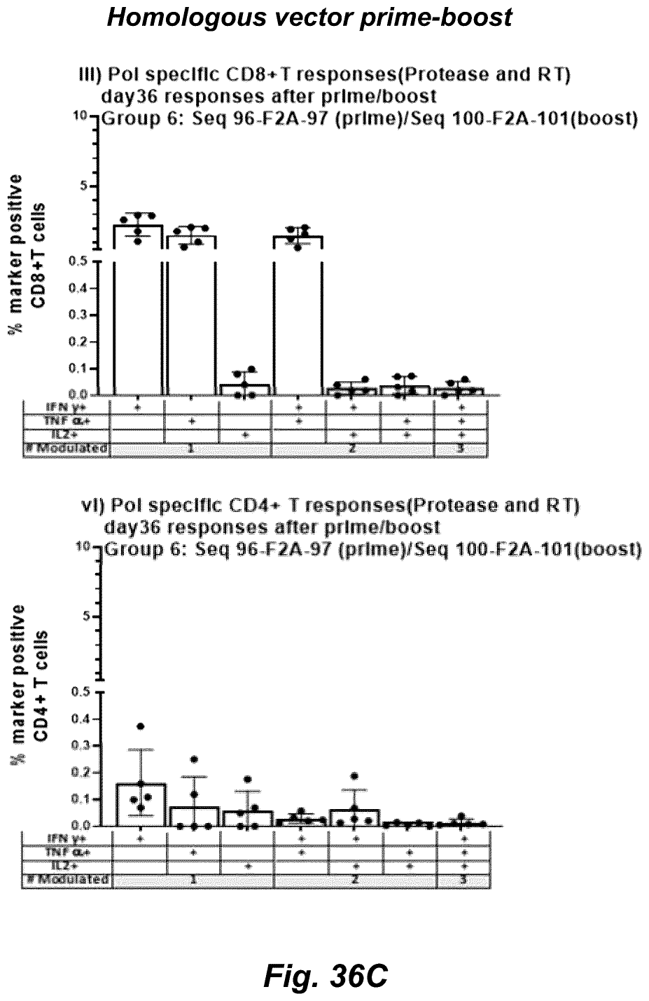

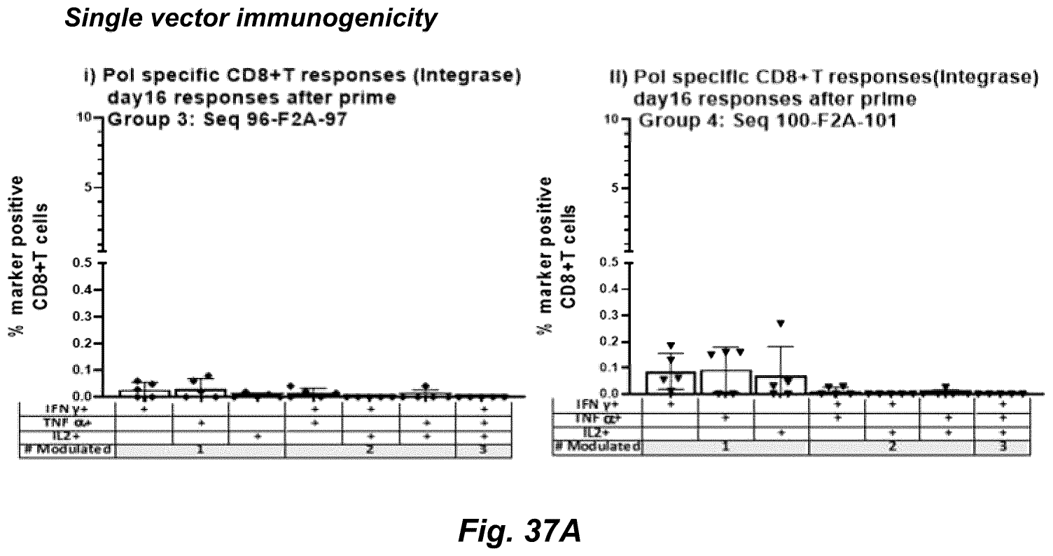

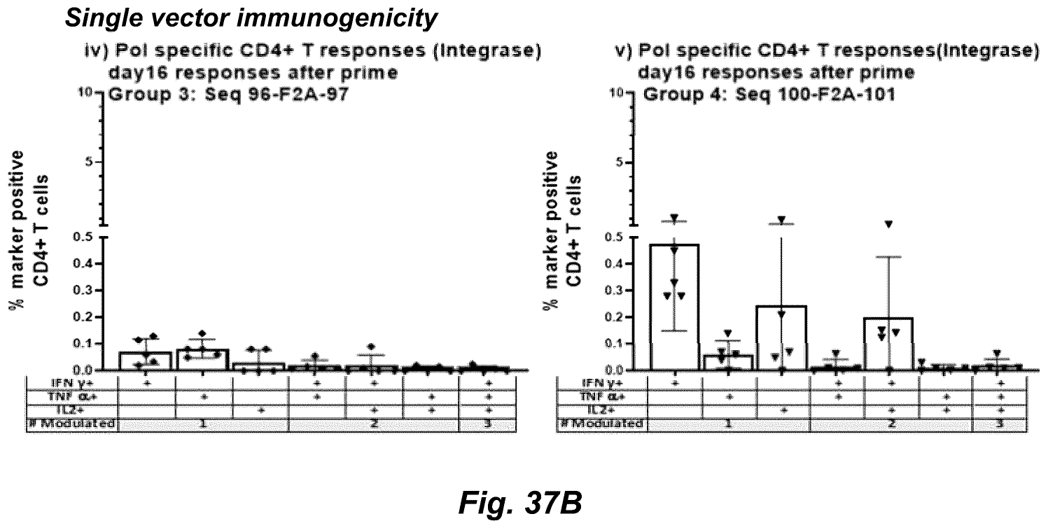

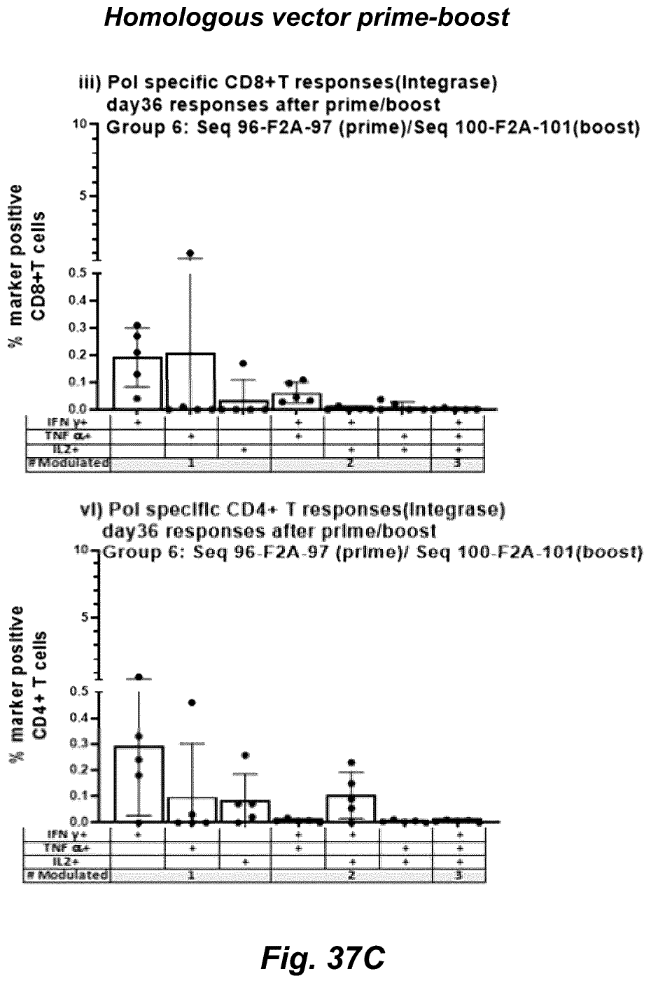

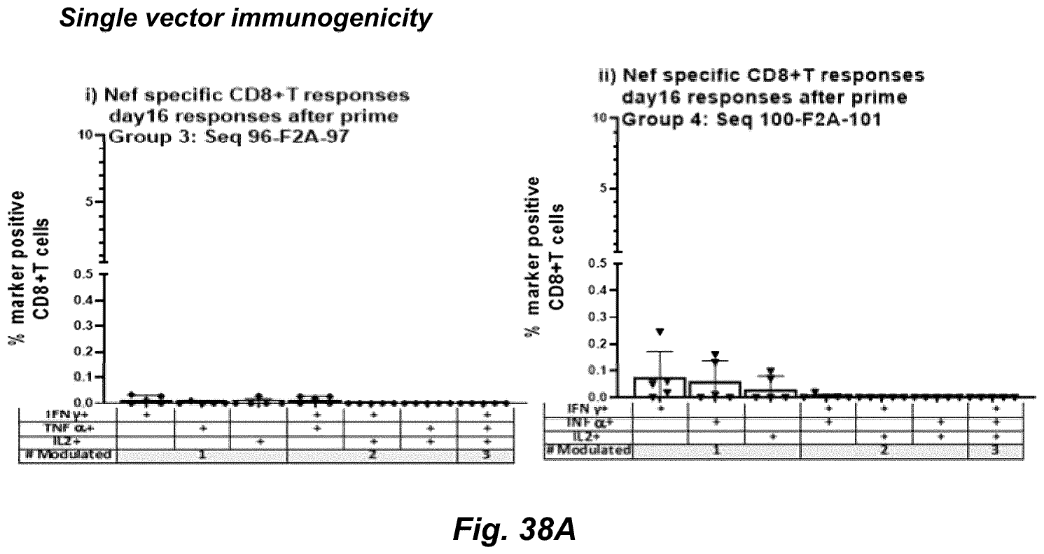

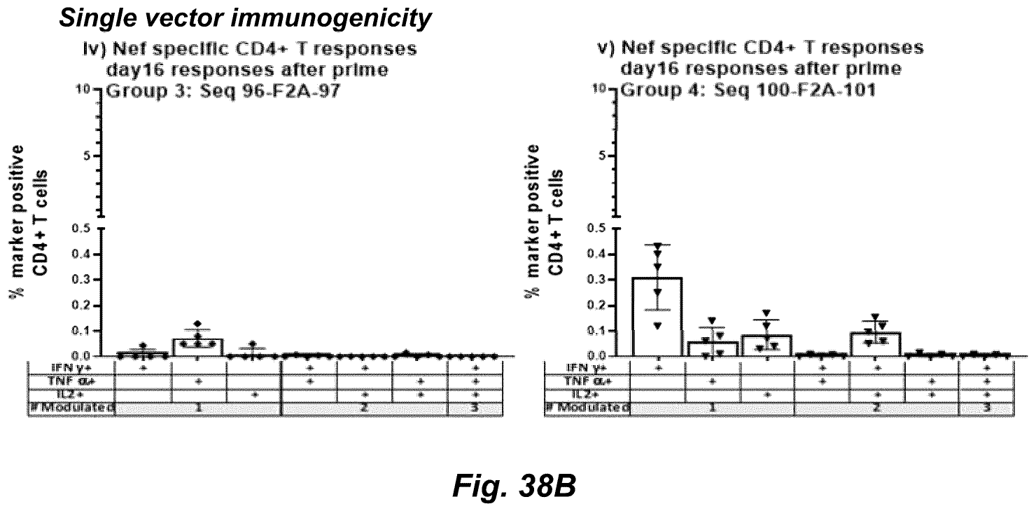

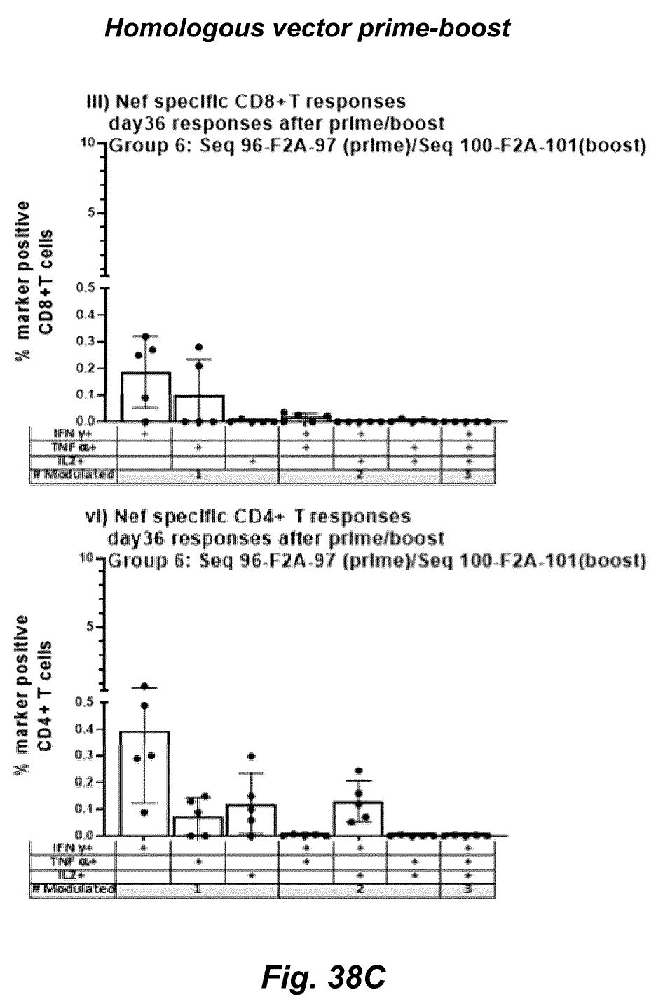

FIGS. 36-38 show immunogenicity against HIV-1 Pol and Nef antigens by intracellular cytokine staining (ICS) following single vector immunization with Seq 96-F2A-97 (tPA-SEQ ID NO: 107), or Seq 100-F2A-101 (tPA-SEQ ID NO: 111), and by homologous vector prime-boost immunization. The Y axis represents proportion of CD8+ (i, ii and iii) or CD4+ (iv, v and vi) T cells exhibiting HIV-1 Pol (protease and RT) (FIG. 36), Pol (integrase (FIG. 37) and Nef (FIG. 38) specific responses by expressing cytokines IFN-γ, IL-2 and TNF-α, either as individual cytokines or combination of cytokines, as shown on X axis. Antigen specific values were obtained by subtracting no antigen stimulated control to exclude nonspecific responses. HIV-1 Pol (Protease and RT) specific CD4+ and CD8+ T cell responses were seen in response to vaccination with robust CD8+ T cell responses. Seq 100-F2A-101 (tPA-SEQ ID NO: 111) demonstrated strong immunogenicity with polyfunctionality (>2 cytokines produced). Low levels of Pol (Integrase) and Nef specific CD4+ and CD8+ T cell responses were generated in response to vaccination and higher responses were seen with CD4+T cells. Seq 100-F2A-101 (tPA-SEQ ID NO: 111) demonstrated and induced mostly monokine production. Homologous vector prime-boost immunization with the vectors encoding the fusion polypeptides enhanced both CD4+ and CD8+ T cell responses.

The data are consistent with the conclusion that the above tested HIV-1 bivalent sequences are immunogenic either as individual priming sequences, or when combined with an F2A linker, except for the Seq 96-F2A-Seq97 (tPA-SEQ ID NO: 107), and also in a homologous prime boost immunization. The magnitude of responses generated were antigen specific, CD4+ and CD8+ and expressed polyfunctionality.

### Example 9

### In Vitro Assays Demonstrating Human T Cell Activation Induced by Immunogens 1-3

In this example, we established an in vitro method for testing the efficacy of T cell priming in humans by vaccine constructs in expression vectors. The application of this method in vaccinology allows evaluation of antigen processing, presentation and priming of T cells in humans of the transgene cassette, as well as the study of immune parameters including adjuvants and immune modulators that may modify the efficacy of priming.

### Methods

Monocyte purification and maturation of monocyte derived dendritic cells (mol) ('s). Freshly isolated or cryopreserved PBMCs were used in the moDC-based T cell stimulation assays. CD14+ monocytes were purified from PBMCS from individuals with or without HIV, and ART naïve or on ART using the EasySep human anti-CD14 positive selection antibody kit (StemCell Technologies). Flow cytometry was used to confirm the purification of the isolated CD14+ monocytes to >90% prior to the establishment of the culture. To generate immature moDCs, 2×10⁶ purified CD14+ monocytes were cultured in 3 mL of moDC differentiation media (complete RPMI 1640 containing 10% heat inactivated fetal calf serum, 1% penicillin streptomycin/mL, 0.5 mM HEPES, 800U/mL of GM-CSF (Miltenyi Biotec), and 1000U of IL-4 (Miltenyi Biotec)) in 6 well culture plates. The plates were incubated at 37° C. and 5% CO2 for 6 days and monitored daily to ensure adherence of monocytes. To generate mature moDCs, adherent immature moDC cultures were supplemented with recombinant soluble CD40L (0.5 ug/ml), IFN-γ (1,000U/ml), PGE2 (5 μM), TNF-α (10 ng/ml), IL-6 (100 ng/ml) and IL-1B (10 ng/ml) with an additional 3 ml of moDC differentiation media on day 6 and incubated at 37° C. and 5% CO₂ for an additional 48 hrs.

On day 8, adherent mature moDCs were detached using ice-cold PBS and a cell scrapper to manually detach the moDCs. Following this procedure, unattached cells were washed using moDC differentiation media and transferred to a 50 ml centrifuge tube. The resulting cell mixture was centrifuged at 1500 rpm for 5 minutes at room temperature. Next, the supernatant was discarded and the cell pellet was resuspended in 5 ml of moDC differentiation media. A fraction of the mature moDCs were isolated and stained to characterize the differentiation phenotype of the moDCs with antiCD11c+, anti-HLA-DR+, anti-CD14-, anti-CD40+, anti-DCSIGN+, anti-CD83, anti-CD86 and anti-OX40L antibodies.

Transduction of moDC's with Adenovirus 5 (Ad5) viral vectors. The purified moDCs were harvested, washed twice in serum-free media, and re-suspended in X-Vivo 15 (BioWhittaker, Walkersville,MD) at 107/ml. Cells were equilibrated at 37° C. in a water bath for 20-30 min before transduction. Ad5 stocks expressing vaccine immunogen or empty vector controls were thawed on ice and added to the moDC suspension at a multiplicity of infection (MOI) of 500. Cells were gently mixed and placed immediately in the 37° C. incubator. After 2 hours, warm moDC differentiation media containing GM-CSF and IL-4 were added to dilute the moDCs to a final concentration of 106/ml. Transduced moDCs (4×106) were transferred to T75 cell culture flasks and maintained at 37° C. in 5% CO₂ for an additional 48 h before addition of autologous PBMCs.

Co-culture of autologous PBMC's with mol) C's. In experiments evaluating the immunogenicity of our conserved regions vaccines, autologous PBMCs were enumerated and 80×106 PBMCs were co-cultured with 4×106 moDCs that had been transduced with Ad5 vectors expressing vaccine immunogens or Ad5 empty vector controls. The PBMC-moDC co-cultures proceeded for a period of 10 days in the presence of IL-2 (50U/ml), IL-7 (10 ng/ml), Efavirenz (0.1 μM) and Elvitegravir (0.1 μM). Co-cultures of moDC and PBMCs were set up at a moDC: PBMC ratio of 1:20.

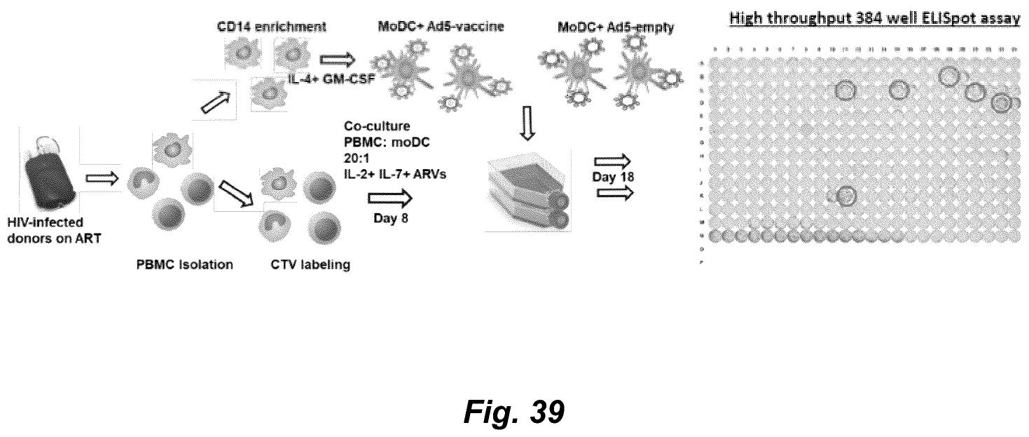

IFN-γ ELISpot Assays. Pre-coated strip ELISpot plates (Cellular Technologies Limited) were used for all ELISpot analyses. Briefly, 3×10⁴ cells from Day 10 moDC-PBMC cultures were seeded to each well. Vaccine-matched peptides consisting of 15-mers overlapping by 11 amino acids spanning the entire HIV conserved regions immunogen were assembled into 384 ELISpot plates with each individual well corresponding to an individual 15-mer peptide and used in IFN-γ ELISpot assays to evaluate vaccine immunogenicity. For positive controls, 50 ng/ml PMA (Sigma) was added. Plates were incubated at 37° C. in 5% CO₂ for 24 h. After 24 h stimulation, the cells were removed from the plates and the wells were washed three times in PBS prior to three washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming units (SFUs) were counted on an Immunospot ELISpot reader. The settings were identical for all plates and counts were expressed at SFU per 3×10+PBMCs. The SFU were calculated as number of spots in test wells minus the mean number of spots in medium control wells. Positive responses were defined as >3-fold higher SFUs compared to medium control wells and >5 spots per well (3×10⁴ cells). Medium control wells contained media reconstituted with a similar composition of DMSO as peptide stimulated test wells. A schematic summarizing the moDC-PBMC culture and ELISpot assay is depicted in FIG. 39.

In vitro viral inhibition assay. The capacity of vaccine-induced CD8+T cells to suppress HIV-1 infection of autologous CD4+T cells was evaluated to determine cytotoxicity. CD4+ T cells were isolated using negative magnetic bead selection (StemCell Technologies) from cryopreserved PBMCs, rested for 24 h and cultured in RPMI and 10% FBS. After 24 h, cultured CD4+ T cells were washed, counted and added to 50 ml conical tubes for spinoculation with HIV-1BaL at a multiplicity of infection (MOI) of 0.01. Spinoculation was performed by centrifugation at 1200g for 2 h. After infection, CD4+ T cells were washed twice and cultured in in RPMI and 10% FBS and IL-2 (30U/ml) for 72 h. After 72 h, CD8+ T cells were isolated at the end of the PBMC-moDC co-cultures using negative magnetic bead selection (StemCell Technologies), labeled with CFSE and counted. Meanwhile, cultured CD4+ T cells were washed, counted and plated in U-bottom 96 well plates with vaccine-induced CD8+T cells at a 1:1 ratio in RPMI and 10% FBS. Three days post co-culture of CD4+ T cells and CD8+T cells, cells were stained with viability dye and surface markers, followed by intracellular detection of HIV-1 Gag (Beckman Coulter) using the IC Fixation/Permeabilization kit (BD Biosciences) according to the manufacturer's protocol. Cells were incubated with a mixture of fluorescence-conjugated anti-human antibodies for 30 min at 4° C. Stained cells were washed twice using FACS buffer (PBS, 2% FCS, 0.1% NaN3), acquired with an LSR II flow cytometer using FACSDiva software (BD), and analyzed using FlowJo software version 10.2 (TreeStar). For surface staining, cells were stained with anti-CD4 BV605 clone OKT4, anti-CD8 BV650 clone RPA-T8, anti CD3 AF700 clone SK7, anti-CD20 BV421 clone 2H7, Live-dead Aqua dye (ThermoFischer). For intracellular detection of p24, cells were fixed and permeabilized using Cytofix/cytoperm buffers (BD Biosciences) and stained with anti-HIV Gag p24 PE (KC57). All experiments were performed in duplicate or triplicate, depending on cell availability. Uninfected CD4+ T cells were included as negative controls and infected CD4+T cells cultured without CD8+T cells served as 100% infectivity controls. Productive infection was considered only when >0.1% p24 Gag+CD4-cells were detected by flow cytometry for each independent sample.

### Results

In this example, we used the in vitro T cell priming assay described herein to evaluate immunogenicity and decode the CD8+ T cell responses to the vaccine immunogen (schematic provided in FIG. 39). We focused on determining the epitopes within conserved regions vaccine that induce antigen specific T cell responses and evaluated the impact of pre-existing responses on induction of de novo responses. Monocyte derived DCs were transduced with viral vectors containing a vaccine transgene were able to prime autologous vaccine antigen specific T cells in vitro. This assay can facilitate the preclinical evaluation of vaccine constructs and provides a useful tool for the identification of immunogenic regions within the conserved region vaccine across a broad repertoire of participants prior to initiation of large-scale vaccine trials.

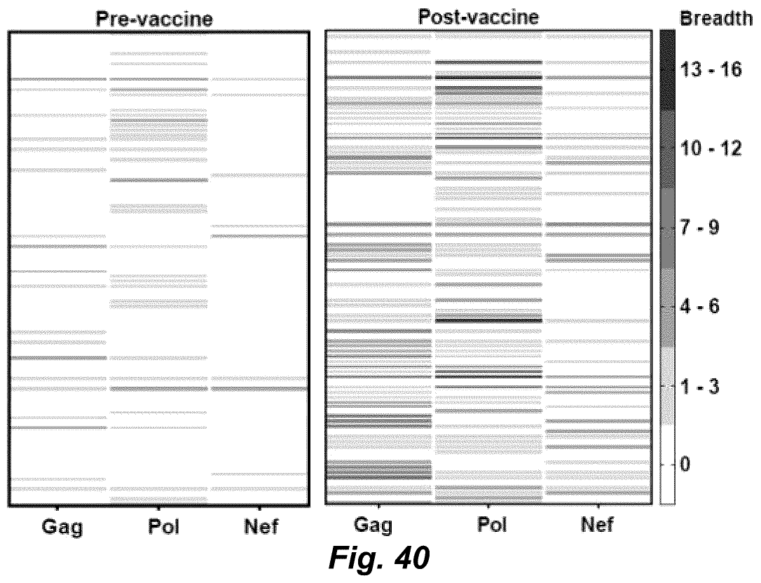

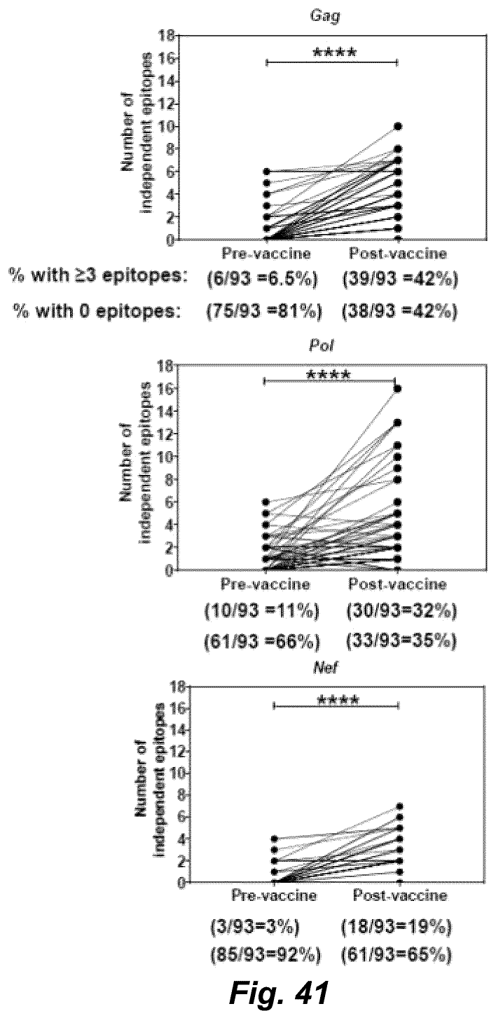

We completed the evaluation of immunogenicity for immunogen 1 (SEQ ID NOs: 105, 107, 109 and 111) in N=93 HIV-1+participant samples. Patient to patient variability is observed in transduction efficiency of moDCs and may reflect variability in expression of receptors to facilitate uptake of viral vectors as would be expected in a heterogeneous human population (FIGS. 40-41).

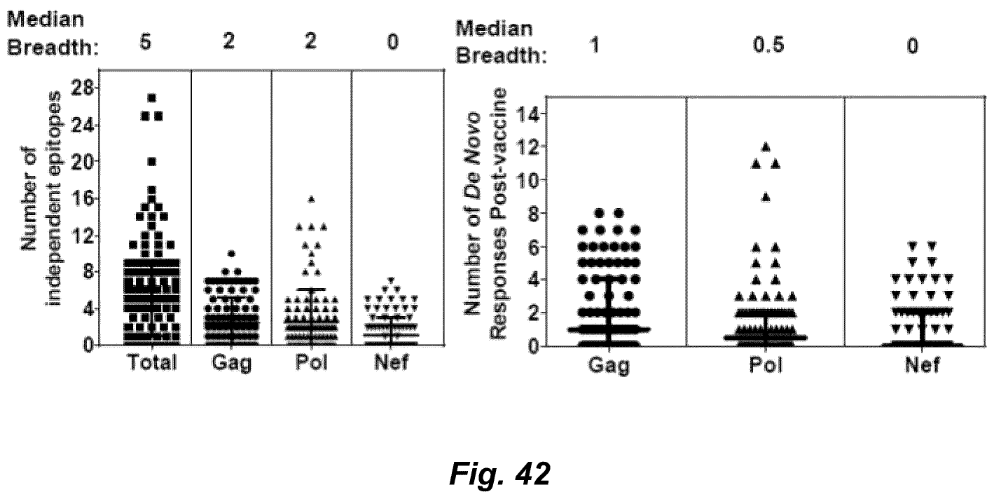

The heat map depicted in FIG. 40 summarizes the profile of the breadth of immune responses to each HIV protein in all 93 participants after in vitro priming with SEQ ID NOs: 105, 107, 109 and 111. Our data shows that in vitro vaccination resulted in induction of responses to conserved regions (>1 epitope) in 80% (74/93) of tested samples, suggesting that vaccination re-focuses immune responses to conserved regions. Additionally, while vaccination with conserved regions vaccine boosted 25% of pre-existing detectable responses, 69% of all responses identified post vaccination were de novo T cell responses primarily to conserved Gag and Pol epitopes that were not detected in the pre-vaccine condition (Ad5-empty vector control) (FIG. 42). Vaccination also significantly enhanced the breadth of immune responses to Gag, Pol and Nef antigens (median total breadth of responses-5, median Gag breadth=2, median Pol breadth=2 and median Nef breadth=0; FIG. 40). Detailed analysis further showed that vaccination significantly enhanced the fraction of participants responding to ≥3 epitopes within each of Gag, Pol and Nef (FIG. 41). We chose to evaluate responses that were greater than 3 epitopes given the data from the STEP Trial. See, Janes, et al., J Infect Dis (2013) 208 (8): 1231-1239; ClinicalTrials.gov identifier: NCT00095576.

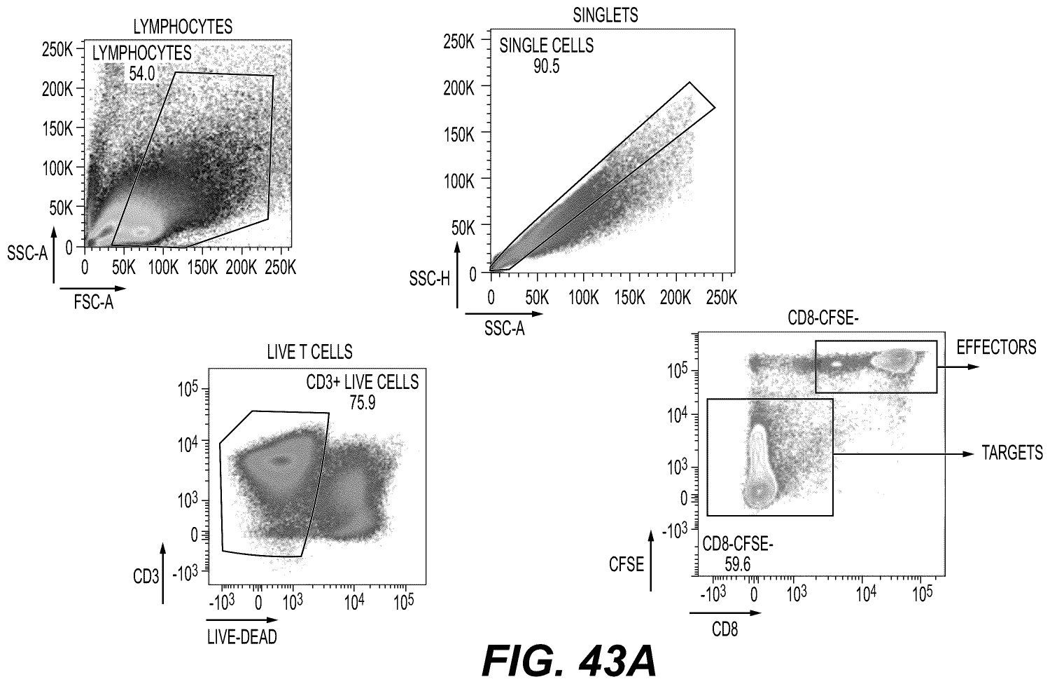

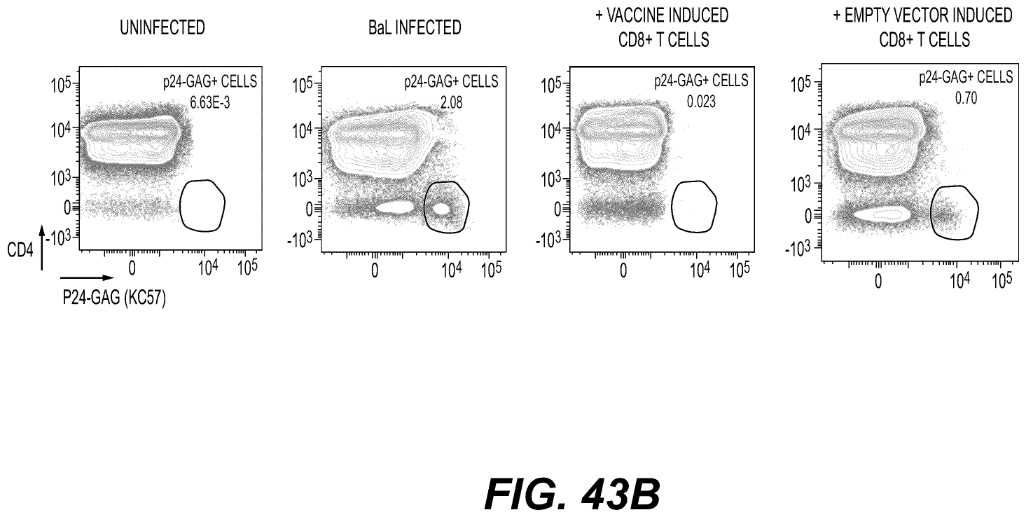

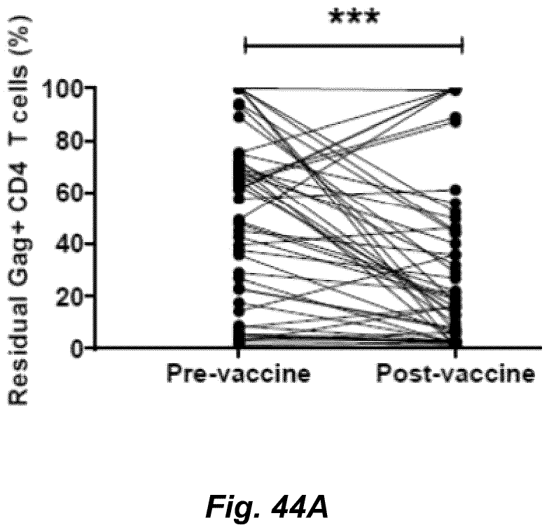

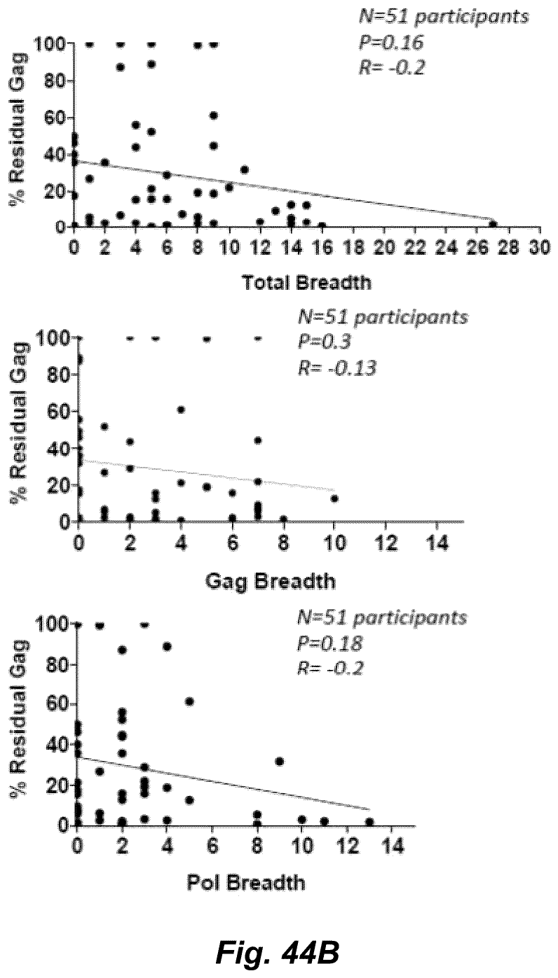

To determine whether vaccine-induced CD8+T cells could eliminate HIV-1 infected cells in vitro, we performed HIV-1 viral inhibition assays. CD4+ T cells from participants were infected with HIV-1BaL and co-cultured either alone or in the presence of purified vaccine or empty vector primed CD8+T cells. Data from 51/68 participants who demonstrated a productive infection rate with HIV-1BaL (≥0.1% p24 Gag+ cells) were used in our analysis (FIG. 43). The data in FIG. 44B shows that CD8+ T cells from 69% (N=35/51) of participants demonstrated increased suppression of HIV-1BaL in vitro in the presence of vaccine (SEQ ID NOs: 105, 107, 109 and 111) primed CD8+T cells compared to empty vector primed CD8+T cells. To evaluate whether there was a correlation between breadth of responses and CD8+T cell mediated suppression of HIV-1BaL, we evaluated breadth and cytotoxicity in each participant that had demonstrated a productive infection rate with HIV-1BaL (≥0.1% p24 Gag+ cells). The data in FIG. 44B shows a lack of correlation between % residual Gag+ cells with Gag/Pol breadth which may be indicative that the quality of responses was more important.

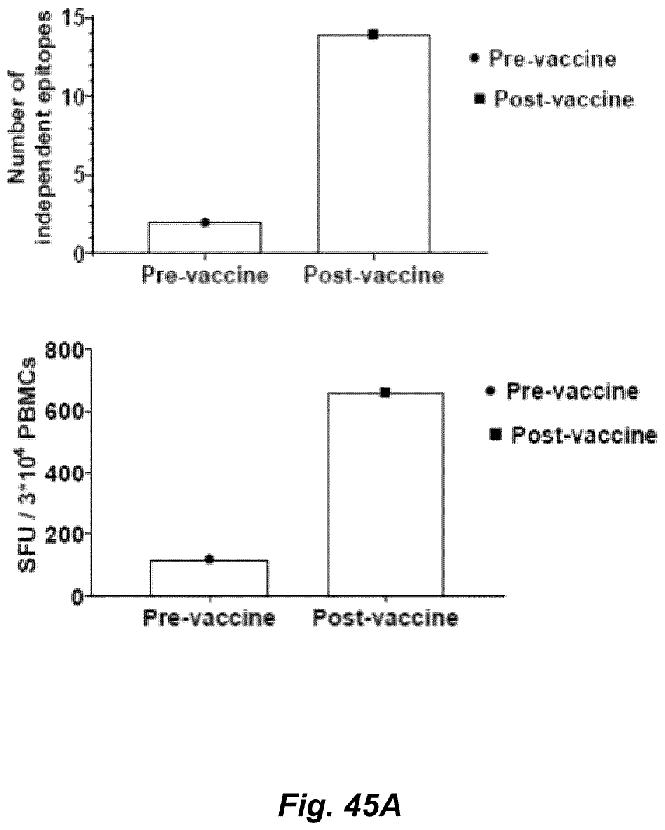

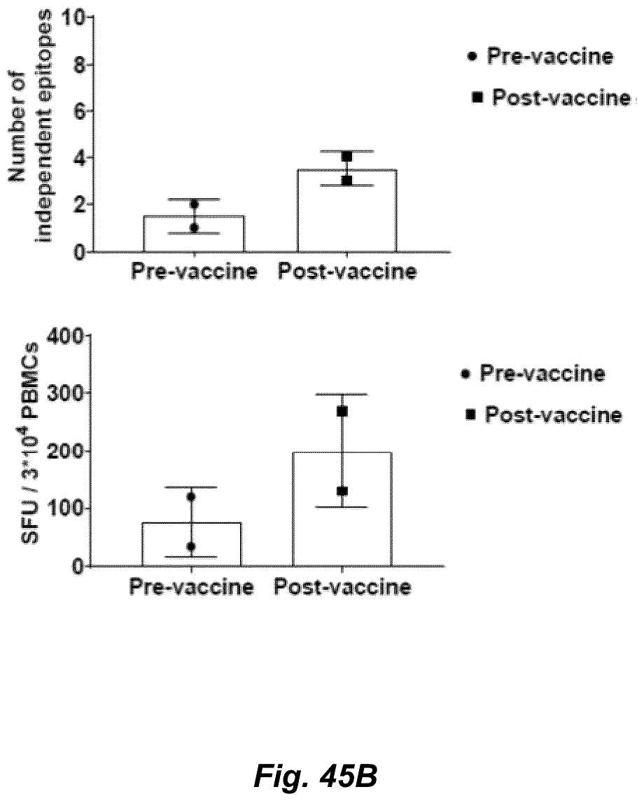

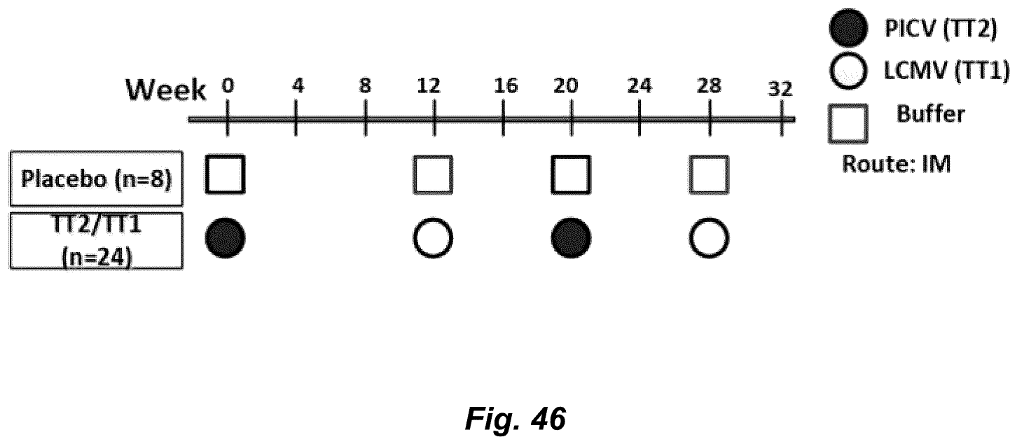

We also evaluated immunogenicity of immunogen version 2 (SEQ ID NOs: 82-89) and immunogen version 3 (SEQ ID NOs: 90-93), using the moDC-T cell priming assay described above, in N=3 participants. The data, summarized in FIGS. 45A and 45B, show the breadth and magnitude of total, Gag, Pol and Nef immune responses to each immunogen. For immunogen version 2, in a single donor in vitro priming with the vaccine sequence resulted in expansion of the response from 2 to 14 independent epitopes; for immunogen version 3, the median breadth of the response was 3.

### Example 10

### Non-Human Primate (NHP) Arenavirus Vector Data

Heterologous prime-boost in non-human primates. Simian immunodeficiency virus infection in NHPs serves as a good model for evaluating HIV vaccine vector immunogenicity. To evaluate the immunogenicity of replication-competent LCMV (TT1) and replication-competent PICV (TT2) vectors in NHPs (e.g., vectors as described in WO2016075250 and WO2017198726), we developed vectors expressing full length SIV proteins Gag, Env or Pol derived from the SIVsme543 strain. The amino acid sequences are provided in Table 2. Indian-origin healthy rhesus macaques were immunized via IM route with the arenavirus vectors. The Gag and Env expressing vectors (replication attenuated Pichinde arenavirus (TT2) and replication attenuated LCMV arenavirus (TT1)) were administered on the left quadricep whereas the Pol expressing vectors (TT2 and TT1) were administered on the right quadricep. The doses administered are as below: 1×10⁶ RCV of TT2 Gag, 1×10⁶ RCV of TT2 Env, 1×10⁶ RCV of TT2 Pol1/Pol2, 4×10⁶ RCV of TT1 Gag, 4×10⁶ RCV of TT1 Env, and 2×10⁶ RCV of TT1 Pol1/Pol2. In the placebo group, NHPs were administered placebo buffer solution composed of 10 mM HEPES, 150 mM NaCl, 20 mM Glycine and 0.1% macaque serum albumin.

TABLE 2
amino acid sequences of full length SIV proteins
Gag, Env or Pol derived from the SIVsme543 strain

| SEQ | | |
|---|---|---|
| ID | SIV | |
| NO: | protein | Amino acid sequence |
| 228 | SIV GAG | MGARNSVLSGKKADELEKIRLRPNGKKKYML |
| | | KHVVWAANELDRFGLAESLLDNKEGCQKILS |
| | | VLAPLVPTGSENLKSLYNTVCVIWCIHAEEK |
| | | VKHTEEAKQIVQRHLVVETGTADKMPATSRP |
| | | TAPPSGRGGNYPVQQVGGNYVHLPLSPRTLN |
| | | AWVKLVEEKKFGAEVVPGFQALSEGCTPYDI |
| | | NQMLNCVGEHQAAMQIIREIINEEAADWDLQ |
| | | HPQPGPLPAGQLREPRGSDIAGTTSTVEEQI |
| | | QWMYRQQNPIPVGNIYRRWIQLGLQKCVRMY |
| | | NPTNILDVKQGPKEPFQSYVDRFYKSLRAEQ |
| | | TDPAVKNWMTQTLLIQNANPDCKLVLKGLGM |
| | | NPTLEEMLTACQGIGGPGQKARLMAEALKEA |
| | | LRPDQLPFAAVQQKGQRRTIKCWNCGKEGHS |
| | | ARQCRAPRRQGCWGCGKTGHVMAKCPERQAG |
| | | FLGFGPWGKKPRNFPMAQMPQGLTPTAPPED |
| | | PAVDLLKNYMKMGRKQRENRERPYKEVTEDL |
| | | LHLNSLFGEDQ |
| 229 | Pol1- | PQFSLWRRPVVTAYIEEQPVEVLLADDSIVI |
| | Protease/ | GIELGPNYTPKIVGGIGGFINTKEYKDVKIK |
| | RT | VLGKVIKGTIMIGDTPINIFGRNLLTAMGMS |
| | | LNFPIAKVEPIKVTLKPGKEGPKLRQWPLSK |
| | | EKIIALREICEKMEKDGQLEEAPPINPYNTP |
| | | TFAIKKKDKNKWRMLIDFRELNKVTQDFTEV |
| | | QLGIPHPAGLAKRRRITVLDVGDAYFSIPLD |
| | | EEFRQYTAFTLPSVNNAEPGKRYIYKVLPQG |
| | | WKGSPAIFQYTMRNVLEPFRKANPDVTLIQI |
| | | LIASDRIDLEHDRVVLQLKELLNGIGFSTPE |
| | | EKFQKDPPFQWMGYELWPTKWKLQKIELPQR |
| | | ETWTVNDIQKLVGVLNWAAQIYPGIKTKHLC |
| | | RLIRGKMTLTEEVQWTEMAEAEYEENKIILS |
| | | QEQEGCYYQEGKPIEATVIKSQDNQWSYKIH |
| | | QEDKVLKVGKFAKVKNTHINGVRLLAHVVQK |
| | | IGKEALVIWGEVPKFHLPVEREIWEQWWTDY |
| | | WQVTWIPDWDFVSTPPLVRLVFNLVKEPIQG |
| | | AETF |
| 230 | Pol2- | YVDGSCNRQSREGKAGYVTDRGRDKAKLLEQ |
| | RNaseH/ | TTNQQALEAFYLALADSGPKANIIVDSQYVM |
| | Integrase | GIVAGQPTESESRLVNQIIEEMIKKEAIYVA |
| | | WVPAHKGIGGNQEVDHLVSQGIRQVLFLEKI |
| | | EPAQEEHEKYHSNVKELVFKFGIPRLVAKQI |
| | | VDTCDRCHQKGEAIHGQVNAELGTWQMCTHL |
| | | EGKIIIVAVHVASGFIEAEVIPQETGRQTAL |
| | | FLLKLAGRWPITHLHTNGANFTSQEVKMVAW |
| | | WAGIEQTFGVPYNPQSQGVVAMNHHLKTQID |
| | | RIREQANSVETIVLMAVHCMNFKRRGGIGDM |
| | | TPAERLVNMITTEQEIQFQQSKNSKFKNFRV |
| | | YYREGRDQLWRGPGELLWKGEGAVILKVGTE |
| | | IKVVPRRKAKIIKDYGGGKELDSGSHLEDTG |
| | | EAREVA |
| 231 | SIV sm543 | MGCLGNQLLIALLLVSVLEICCVQYVIVFYG |
| | Env | VPAWKNATIPLFCATRNRDTWGTTQCLPDND |
| | | DYSELAVNITEAFDAWNNTVTEQAIEDVWNL |
| | | FETSIKPCVKLTPLCIAMRCNKTETDRWGLT |
| | | GRAETTTTAKSTTSTTTTTVTPKVINEGDSC |
| | | IKNNSCAGLEQEPMIGCKFNMTGLKRDKKIE |
| | | YNETWYSRDLICEQPANGSESKCYMQHCNTS |
| | | VIQESCDKHYWDAIRFRYCAPPGYALLRCND |
| | | SNYSGFAPKCSKVVVSSCTRMMETQTSTWFG |
| | | ENGTRAENRTYIYWHGNSNRTIISLNKYYNL |
| | | PINERPKQAWCRFGGNWSEAIQEVKETLVKH |
| | | PRYTGTNDTRKINLTAPAGGDPEVTFMWTNC |
| | | RGEFLYCKMNWFLNWVEDRDQNSNRWKQQKK |
| | | PEQQKRNYVPCHIRQIINTWHKVGKNVYLPP |
| | | REGDLTCNSTVTSLIAEIDWINNNETNITMS |
| | | AEVAELYRLELGDYKLVEITPIGLAPTDVRR |
| | | SLTLSAQSRTLLAGIVQQQQQLLDVVKRQHE |
| | | LLRLTVWGTKNLQTRVTAIEKYLKDQAQLND |
| | | SLVPNWDNMTWQEWEGKVDFLEANITQLLEE |
| | | AQIQQEKNMYELQKLNSWDIFGNWFDLTSWI |
| | | RYIQ |

IFN-γ ELISpot Assays. Pre-coated strip ELISpot plates (MabTech) were used for all ELISpot analyses. Briefly, 2×105 PBMCs isolated from whole blood at each of the timepoints analyzed was seeded to each well. SIV smE543 peptides consisting of 15-mers overlapping by 11 amino acids spanning the SIV immunogens Gag, Env and Pol at a final concentration of lug/ml were assembled into 96-well ELISpot plates. To determine breadth of responses to Gag, Env and Pol, sub-pools of 12, 16 or 23 peptides respectively were tested individually at a final concentration of lug/ml. Each sub-pool had 10 peptides composed of 15-mers overlapping by 11 amino acids. Each sample was tested in duplicates. For positive controls, 5 ug/ml PHA (Sigma) was added. Medium control wells contained cell culture media reconstituted with a similar composition of DMSO as peptide stimulated test wells. Plates were incubated at 37° C. in 5% CO₂ for 20-24 h. After overnight stimulation, the cells were removed from the plates and the wells were washed three times in PBS prior to three washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming units (SFUs) were counted on an Immunospot ELISpot reader. The settings were identical for all plates and counts were expressed at SFU per 2×105 PBMCs. To determine a proper spot count using the CTL Single Color immunospot counting software, multiple parameters were normalized relative to the sample. Signal sensitivity is set to the highest value before non-specific spots begin to appear. Spot size is reduced to the point where all real spots are counted but artifacts are not. Background signal reduction is set to avoid counting artifacts. The SFU were calculated as number of spots in test wells minus the mean number of spots in medium control wells. Positive responses were defined as >3-fold higher SFUs compared to medium control wells and >50 spots per 1×106 PBMCs.

SIV Challenge and Viral Load. All animals in the study were challenged at 4 weeks post the last vaccine dose (i.e., week 32 of study) with a single intravenous (IV) inoculation of a heterologous SIV virus swarm (SIVmac251, 8.19 TCID50). The viral sequence of the challenge virus differs from that in the vaccine sequence (SIVsme543). The estimated AID50 of the SIVmac251 challenge stock is 0.29 TCID50 via the IV route. Post challenge, all animals were monitored for clinical and laboratory progression as well as viral load to determine peak viral load (calculated at 2 weeks post challenge) and set point viral load (calculated over weeks 10-40 post challenge). Plasma SIV viral load as copies/ml was quantified by two-step RT-PCR assay performed in duplicates.

### Results

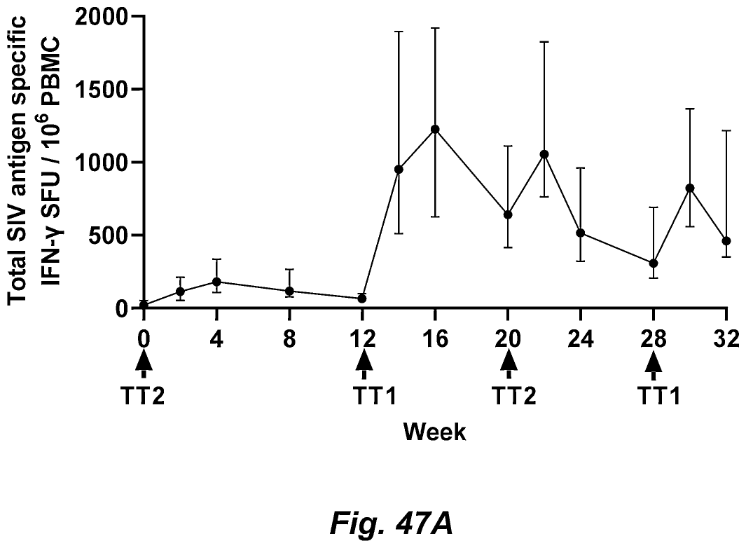

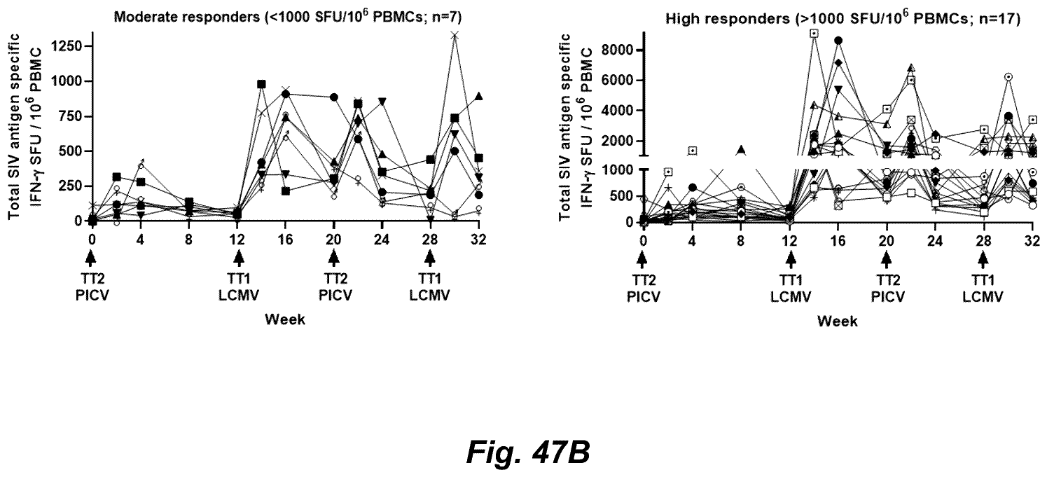

FIG. 47A shows the magnitude of SIV-specific IFN-γ responses as assessed by IFN-γ ELISpot. Boosting by TT1 results in a robust increase in response at week 14. The peak magnitude of response observed at 2 weeks post each vaccine dose is maintained with each subsequent boost.

FIG. 47B shows the categorization of the animals that received the heterologous TT2/TT1 vaccine as moderate or high responders. This is based on the magnitude of peak response post each vaccine dose being less or more than 1000 SFU/million PBMCs respectively. The majority of the animals (17 of 24) were high responders with all animals showing a positive response to at least one SIV immunogen.

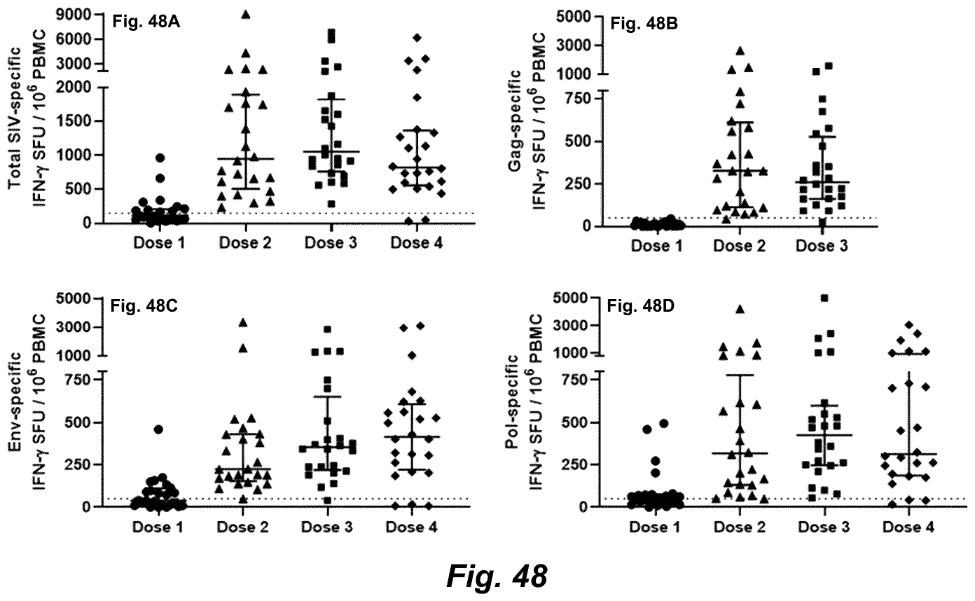

FIGS. 48A-D demonstrate the magnitude of peak SIV-specific IFN-γ responses at 2 weeks post each vaccine dose. Peak Gag- and Pol-specific responses are maintained after doses 2, 3 and 4. Env-specific responses increased with each subsequent dose. After four doses of the heterologous vaccine, a positive response to Gag, Env and Pol is observed in at least 21 of 24 NHPs (response rate=87.5%).

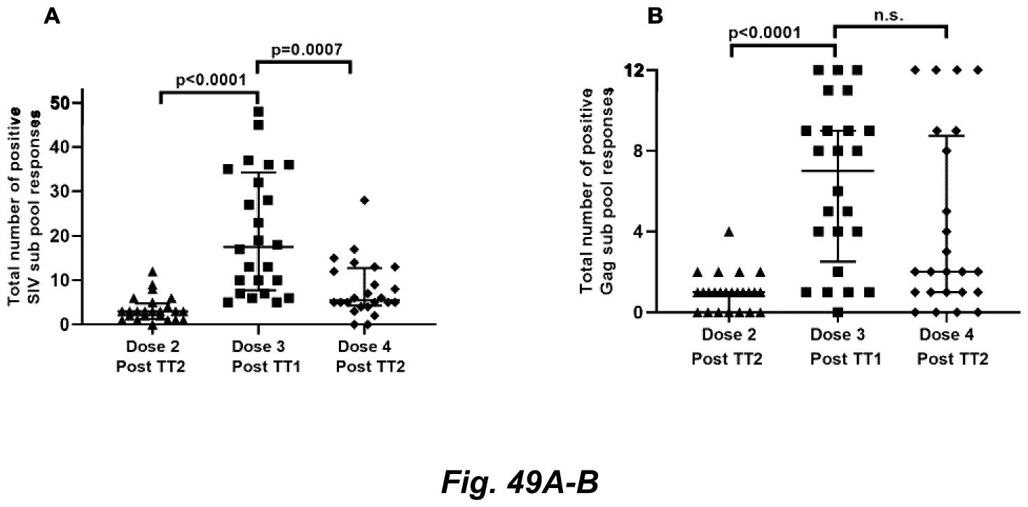

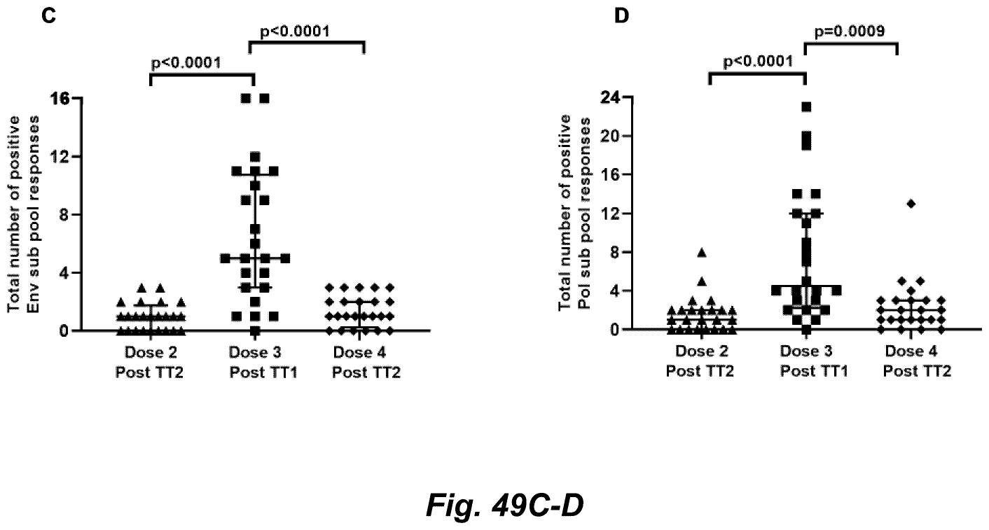

With respect to breadth, highest total SIV-specific breadth was observed post-dose 3 compared to post-doses 2 and 4. Similarly, significantly higher Env- and Pol-specific breadth observed post-dose 3 compared to post dose 2 and 4. Gag-specific breadth is significantly high post dose 3 and 4 compared to post dose 2. These results suggest the induction of TT2/TT1 vaccine-induced breadth of responses to Gag, Env and Pol-specific immunogens at 2 weeks post each vaccine boost dose. See, FIGS. 49A-D.

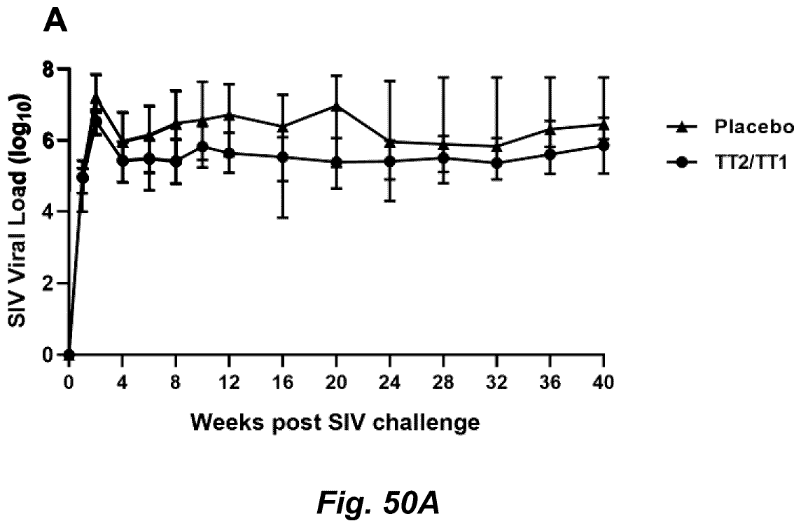

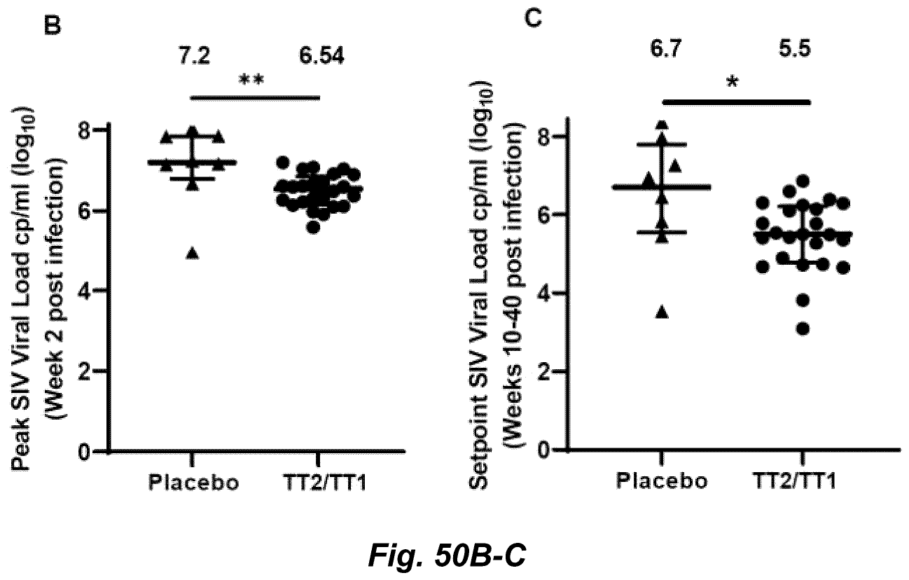

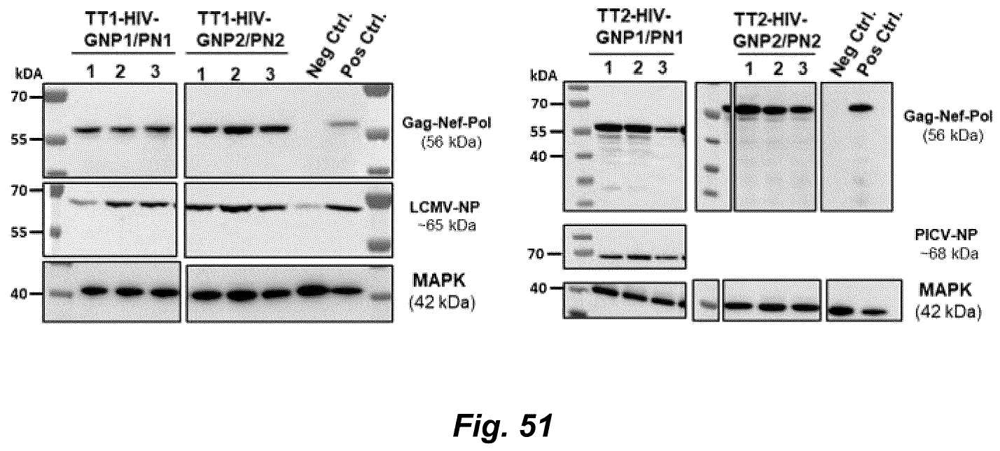

With respect to SIV challenge and viral load, post-challenge, reduced viral load was observed in the TT2/TT1 group compared with the placebo group (FIG. 50A). The median peak viral load measured at 2 weeks post challenge with SIVmac251 was 6.54 log 10 SIV copies/ml in the TT2/TT1 group and 7.2 log 10 SIV copies/ml in the placebo group. A significant decrease in peak viral load (** p=0.0046, Mann-Whitney t-test) was observed in the TT2/TT1 vaccinated NHPs compared to the placebo group (FIG. 50B). The setpoint viral load was calculated as the average SIV viral load over weeks 10-40 post challenge. The median setpoint VL was 5.5 log 10 SIV copies/ml in the TT2/TT1 group and 6.7 log 10 SIV copies/ml in the placebo group. A significant decrease of 1.2 log 10 SIV copies/ml in setpoint viral load (* p=0.0291, Mann-Whitney t-test) was observed in the TT2/TT1 vaccinated NHPs compared to the placebo group (FIG. 50C). These data demonstrate the prophylactic efficacy of heterologous TT2/TT1 prime-boost schemes of PICV/LMCV replication-attenuated arenavirus vectors encoding SIV immunogens in a SIV challenge model in NHPs.

### Example 11

### Replication-Attenuated Arenavirus Vectors Containing

### HIV-1 Immunogen Version 1 Fusion Polypeptide-Encoding Transgenes

In this example, we generated replication-attenuated arenavirus-based viral vectors encoding the computationally defined polypeptide antigens containing conserved regions of HIV-1 encoded by Gag, Nef and Pol genes as a transgene. Replication-attenuated arenavirus vectors based on Pichinde Virus (PICV) and Lymphocytic choriomeningitis virus (LCMV) were generated. The polypeptide segments containing conserved regions were concatenated or connected by different approaches including direct fusion, or the addition of various flexible linkers between regions. The transgenes were segregated and encoded on both viral S-Segments (NP-Segment and GP-Segment). In a first replication-attenuated LCMV vector, the NP-Segment was replaced with a polynucleotide encoding a fusion polypeptide of SEQ ID NO: 98 and the GP-Segment was replaced with a polynucleotide encoding a fusion polypeptide of SEQ ID NO: 100. In a second replication-attenuated LCMV vector, the NP-Segment was replaced with a polynucleotide encoding a fusion polypeptide of SEQ ID NO: 99 and the GP-Segment was replaced with a polynucleotide encoding a fusion polypeptide of SEQ ID NO: 101. In a first replication-attenuated PICV vector, the NP-Segment was replaced with a polynucleotide encoding a fusion polypeptide of SEQ ID NO: 94 and the GP-Segment was replaced with a polynucleotide encoding a fusion polypeptide of SEQ ID NO: 96. In a second replication-attenuated PICV vector, the NP-Segment was replaced with a polynucleotide encoding a fusion polypeptide of SEQ ID NO: 95 and the GP-Segment was replaced with a polynucleotide encoding a fusion polypeptide of SEQ ID NO: 97. Schematics of the fusion polypeptides are provided in FIG. 25.

### Methods

Construction and generation of replication-attenuated arenavirus vectors containing HIV-1 fusion polypeptide transgene polypeptide variants. Replication-attenuated arena virus vectors expressing HIV-1 computationally defined vaccine immunogens, were generated as described previously (Kallert, et al, Nat Commun. (2017) 8:15327; see also, WO2016075250 and WO2017198726). Briefly, cDNA sequences were synthesized and subcloned into the respective backbone plasmids. Plasmids for each viral genomic segment and plasmids expressing the viral trans-acting factors NP and L, were transfected into LCMV-GP complementing cells. Cell culture supernatant was harvested and further propagated on HEK293 suspension cells, to generated vector stock material (passage 1 (P1)). The vectors developed for this evaluation are listed in Table 3 and schematically depicted in FIG. 25.

TABLE 3
Arenavirus Vectors Encoding HIV-1 Immunogen 1 Fusion Polypeptides

| | | Fusion Polypeptide | Transgene |
|---|---|---|---|
| Vector Name | Arenavirus | (SEQ ID NOs) | (SEQ ID NOs) |
| TT1-HIV- | LCMV | NP-SEQ ID NO: 98 | NP-SEQ ID NO: 140 |
| GNP1/PN1 | | GP-SEQ ID NO: 100 | GP-SEQ ID NO: 146 |
| TT1-HIV- | LCMV | NP-SEQ ID NO: 99 | NP-SEQ ID NO: 143 |
| GNP2/PN2 | | GP-SEQ ID NO: 101 | GP-SEQ ID NO: 149 |
| TT2-HIV- | PICV | NP-SEQ ID NO: 94 | NP-SEQ ID NO: 131 |
| GNP1/PN1 | | GP-SEQ ID NO: 96 | GP-SEQ ID NO: 135 |
| TT2-HIV- | PICV | NP-SEQ ID NO: 95 | NP-SEQ ID NO: 133 |
| GNP2/PN2 | | GP-SEQ ID NO: 97 | GP-SEQ ID NO: 137 |

Evaluation of target gene expression by detection of the conserved region for Gag (p24). We evaluated transgene expression by detection of the conserved region for Gag (p24) either by Immunoblot or Double-Immuno staining (D1). For immunoblot analysis, infected HEK293 cells were harvested and lysed, before applying whole cell lysates to acrylamide gel electrophoresis. After blotting, membranes were incubated using either a mouse mAb [39/5.4A] to detect HIV1 p24 (Abcam (ab9071)), a rabbit pAb ERK-2 (sc-154; Santa Cruz) or antibodies for detection of successful infection (rabbit pAb anti LCMV-NP (University of Genf; Prof. Doron Merkler); mouse mAb anti PICV-NP (University of Basel; Prof. Daniel Pinschewer).

Evaluation of stable transgene integration by serial passaging of viral vectors. We evaluated transgene stability of replication-attenuated arenavirus vectors, by serial passaging of vectors stock material in HEK293 suspension cells. To this end, P1 stock material infectious titer was determined by Focus Forming Assay, and HEK293 cells were infected using a multiplicity of infection (MOI) of 0.001. Progeny virus was harvested 72 hours post infection and again titrated. Further passages were infected applying the same principle. After a maximum eight passages, virus containing supernatant samples were analyzed by transgene PCR and/or D1 staining. For transgene PCR analysis, RNA from virus containing supernatant was extracted and subsequently reverse transcribed and amplified by PCR using specific HIV-transgene flanking primers. PCR products were applied to gel electrophoresis analysis to evaluate transgene PCR fragment length as an indicator of transgene stability.

### Results

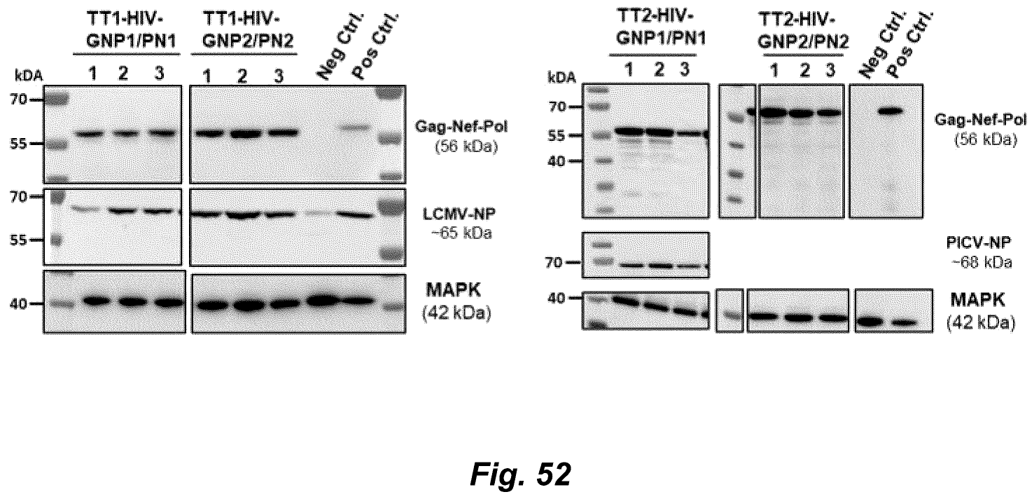

Evaluation of HIV-1 fusion polypeptide transgene expression by immunoblot analysis. We could verify expression of Gag/Nef/Pol antigen by immunoblot analysis of cell lysates of three replicates per vector. The results are shown in FIG. 52.

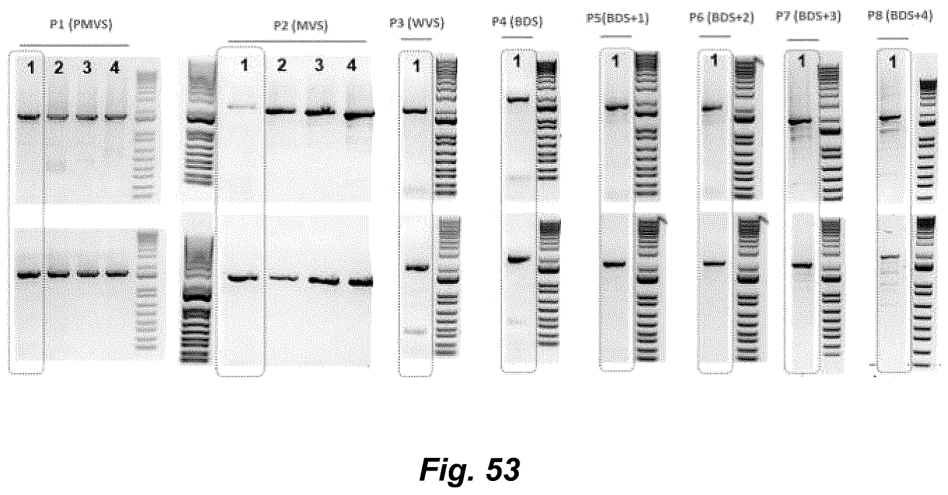

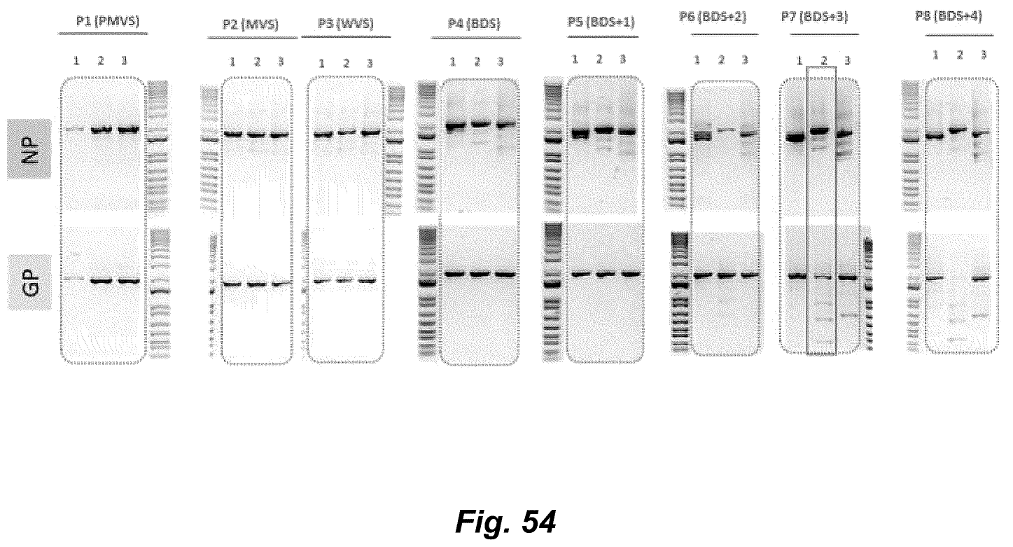

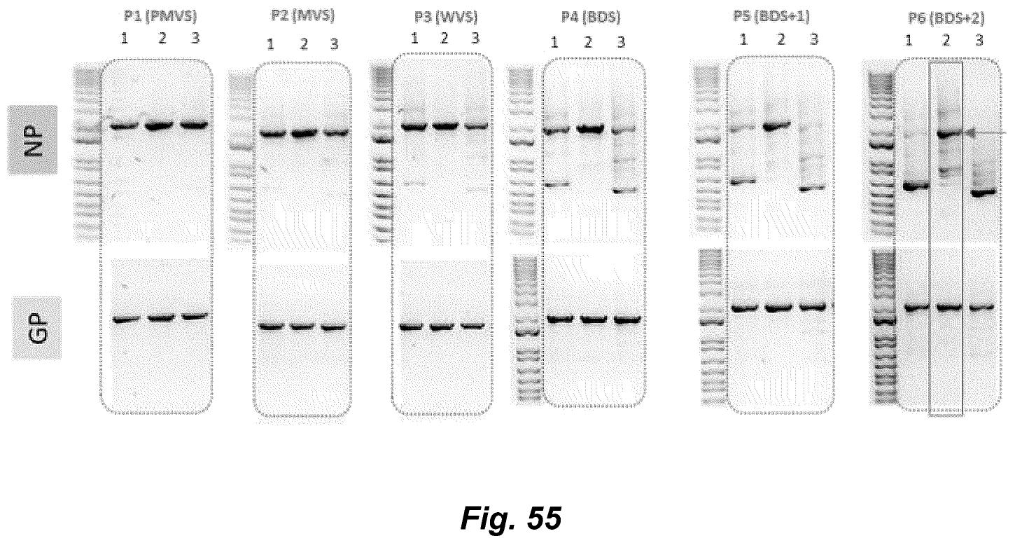

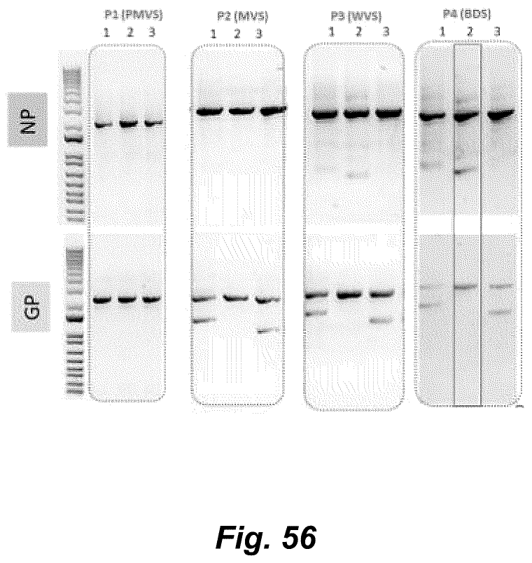

Evaluation of stable integration of HIV-1 fusion polypeptide transgene by serial passaging. To evaluate, whether HIV-1 Immunogen 1 transgenes are stably encoded in replication attenuated arenaviral vectors over several passages, we analyzed cell culture supernatant and performed transgene PCR analysis. The passage level at which the majority (>50%) of the transgene specific band still shows the expected full-length size, was considered the last passage level with stable transgene insertion. Results are shown in Table 4 and FIGS. 53-56. Further, where applicable, supernatant samples harvested from each passaging step were applied to double-immuno staining (D1 staining), to determine the ratio of HIV-1 Gag and arenavirus-NP expression. A combination of both results allowed for a more accurate assessment of transgene stability of HIV-1 Immunogen 1 transgenes.

We found that replication attenuated-LCMV HIV antigen encoding vectors to stably encoded the transgenes up to passage level 7, while replication attenuated-PICV HIV antigen encoding vectors stably encoded the transgenes to passage levels 4 and 6.

TABLE 4
Overview Table for Assessment of Transgene Genetic Stability

| | | Transgene Stability |
|---|---|---|
| Vector Name | Arenavirus | Through Passage (P) |
| TT1-HIV-GNP1/PN1 | LCMV | P7 |
| TT1-HIV-GNP2/PN2 | LCMV | P7 |
| TT2-HIV-GNP1/PN1 | PICV | P6 |
| TT2-HIV-GNP2/PN2 | PICV | P4 |

P# indicates number of passages

Replication-attenuated-LCMV HIV antigen encoding vectors stably retained the transgenes encoding the fusion polypeptides SEQ ID NOs 98 and 100 (TT1-HIV-GNP1/PN1) and SEQ ID NOs: 99 and 101 (TT1-HIV-GNP2/PN2) up to passage level 7. Replication-attenuated-PICV vector TT2-HIV-GNP1/PN1 stably retained transgenes encoding fusion polypeptides SEQ ID NOs: 94 and 96 up to passage level 6 and replication attenuated-PICV vector TT2-HIV-GNP2/PN2 stably retained transgenes encoding fusion polypeptides SEQ ID NOs: 95 and 97 up to passage level 4, however.

### Example 12

### Replication-Attenuated Arenavirus Vectors Containing HIV-1 Immunogen Version 2 Fusion Polypeptide-Encoding Transgenes

In this example, we generated replication attenuated arenavirus based viral vectors encoding shorter computationally defined polypeptide antigen HIV-1 Immunogen 2, containing conserved regions of HIV-1 including Gag, Nef and Pol genes as a transgene. Replication-attenuated arenavirus vectors based on Pichinde Virus (PICV) and Lymphocytic choriomeningitis virus (LCMV) were generated. The polypeptide segments containing conserved regions were concatenated or connected by different approaches including direct fusion, or the addition of various flexible linkers between regions. The transgenes were segregated and encoded on both viral S-Segments (NP-Segment and GP-Segment). In a first replication-attenuated LCMV vector, the NP-Segment was replaced with a polynucleotide encoding a fusion polypeptide of SEQ ID NO: 84 and the GP-Segment was replaced with a polynucleotide encoding a fusion polypeptide of SEQ ID NO: 88. In a second replication-attenuated LCMV vector, the NP-Segment was replaced with a polynucleotide encoding a fusion polypeptide of SEQ ID NO: 85 and the GP-Segment was replaced with a polynucleotide encoding a fusion polypeptide of SEQ ID NO: 89. In a first replication-attenuated PICV vector, the NP-Segment was replaced with a polynucleotide encoding a fusion polypeptide of SEQ ID NO: 82 and the GP-Segment was replaced with a polynucleotide encoding a fusion polypeptide of SEQ ID NO: 86. In a second replication-attenuated PICV vector, the NP-Segment was replaced with a polynucleotide encoding a fusion polypeptide of SEQ ID NO: 83 and the GP-Segment was replaced with a polynucleotide encoding a fusion polypeptide of SEQ ID NO: 87. Schematics of the fusion polypeptides are provided in FIG. 21.

### Methods

Construction and generation of replication attenuated arenavirus vectors containing HIV-1 Immunogen 2 transgene polypeptide variants. Replication-attenuated PICV and replication-attenuated LCMV viral vectors expressing HIV-1 computationally defined vaccine immunogens, were generated as described previously (Kallert, et al, (2017) Nat.Comm., supra). Briefly, cDNA sequences were synthesized and subcloned into the respective backbone plasmids. Plasmids for each viral genomic segment and plasmids expressing the viral trans-acting factors NP and L, were transfected into LCMV-GP complementing cells. Cell culture supernatant was harvested and further propagated on HEK293 suspension cells, to generate vector stock material (passage 1). The vectors developed for this evaluation are listed in Table 5 and schematically depicted in FIG. 21.

TABLE 5
Arenavirus Vectors Encoding HIV-1
Immunogen 2 Fusion Polypeptides

| | | Fusion Polypeptide | Transgene |
|---|---|---|---|
| Vector Name | Arenavirus | (SEQ ID NOs) | (SEQ ID NOs) |
| TT1-HIV(C2)- | LCMV | NP-SEQ ID NO: 84 | NP-SEQ ID NO: 151 |
| GP1/PN1 | | GP-SEQ ID NO: 88 | GP-SEQ ID NO: 153 |
| TT1-HIV(C2)- | LCMV | NP-SEQ ID NO: 85 | NP-SEQ ID NO: 156 |
| GP2/PN2 | | GP-SEQ ID NO: 89 | GP-SEQ ID NO: 159 |
| TT2-HIV(C2)- | PICV | NP-SEQ ID NO: 82 | NP-SEQ ID NO: 150 |
| GP1/PN1 | | GP-SEQ ID NO: 86 | GP-SEQ ID NO: 152 |
| TT2-HIV(C2)- | PICV | NP-SEQ ID NO: 83 | NP-SEQ ID NO: 154 |
| GP2/PN2 | | GP-SEQ ID NO: 87 | GP-SEQ ID NO: 157 |

Evaluation of target gene expression by detection of the conserved region for Gag (p24). We evaluated transgene expression by detection of the conserved region for Gag (p24) either by Immunoblot or Double-Immunostaining (D1). For immunoblot analysis, infected HEK293 cells were harvested and lysed, before applying whole cell lysates to acrylamide gel electrophoresis. After blotting, membranes were incubated using either a mouse mAb [39/5.4A] to detect HIV1 p24 (Abcam (ab9071)), a rabbit pAb ERK-2 (sc-154; Santa Cruz) or antibodies for detection of successful infection (rabbit pAb anti LCMV-NP (University of Genf; Prof. Doron Merkler); mouse mAb anti PICV-NP (University of Basel; Prof. Daniel Pinschewer).

Evaluation of stable transgene integration by serial passaging of viral vectors. We evaluated transgene stability of replication attenuated arenavirus vectors, by serial passaging of vector stock material in HEK293 suspension cells. To this end, P1 stock material infectious titer was determined by Focus Forming Assay, and HEK293 cells were infected using a MOI 0.001. Progeny virus was harvested 72 hours post infection and again titrated. Further passages were infected applying the same principle. After a maximum of eight passages, virus containing supernatant samples were analyzed by transgene PCR and/or D1 staining. For transgene PCR analysis, RNA from virus containing supernatant was extracted and subsequently reverse transcribed and amplified by PCR using specific HIV-transgene flanking primers. PCR products were applied to Gel electrophoresis analysis to evaluate transgene PCR fragment length as an indicator of transgene stability.

### Results

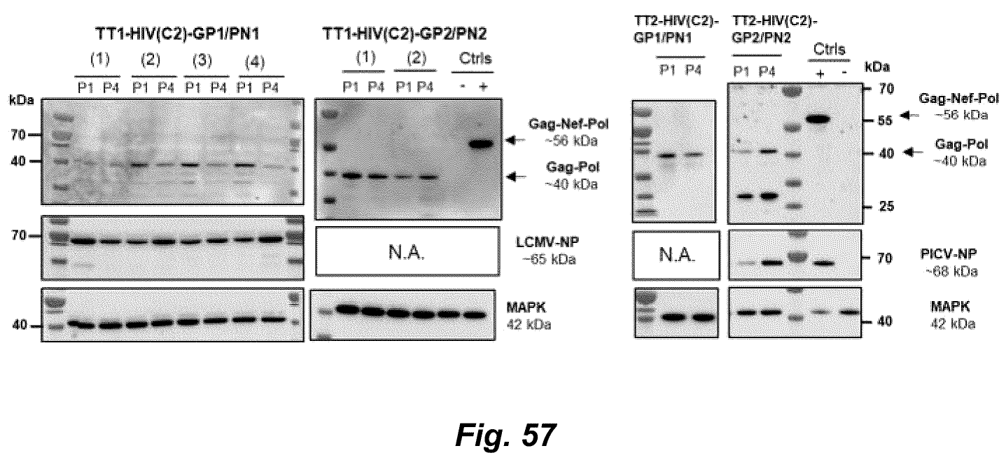

Evaluation of HIV Immunogen 2 transgene expression by immunoblot analysis. We verified expression of Gag/Nef/Pol antigen by immunoblot analysis of cell lysates of four (TT1-HIV (C2)-GP1/PN1) or two (TT1-HIV (C2)-GP2/PN2) replicates per replication-attenuated-LCMV vector (left panel) and one representative vector stock per replication-attenuated-PICV vector (right panel). We determined antigen expression by staining of HIV-1 Gag-Pol in passage level 1 (P1) and 4 (P4). Cells infected with TT2-HIV (C2)-GP2/PN2 showed an additional band around 30 kDa size, when stained by Gag antibody. Results are shown in FIG. 57.

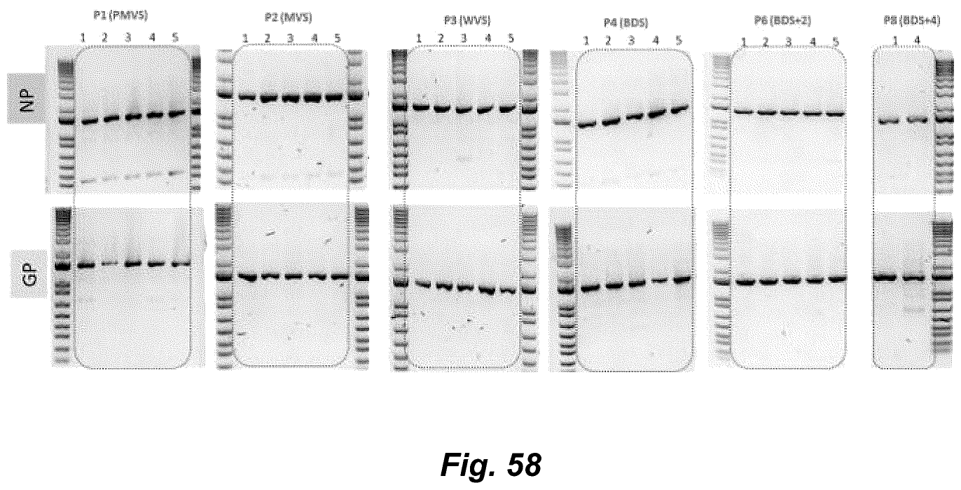

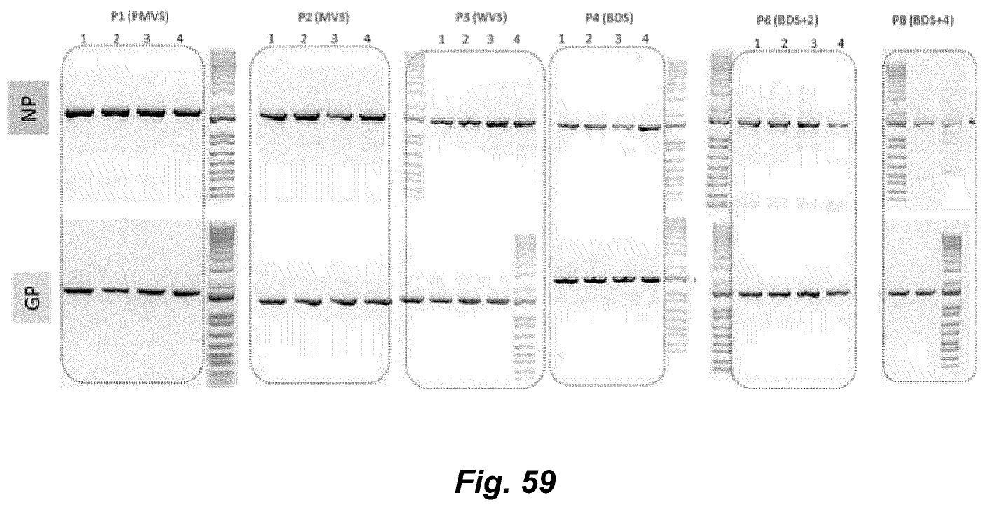

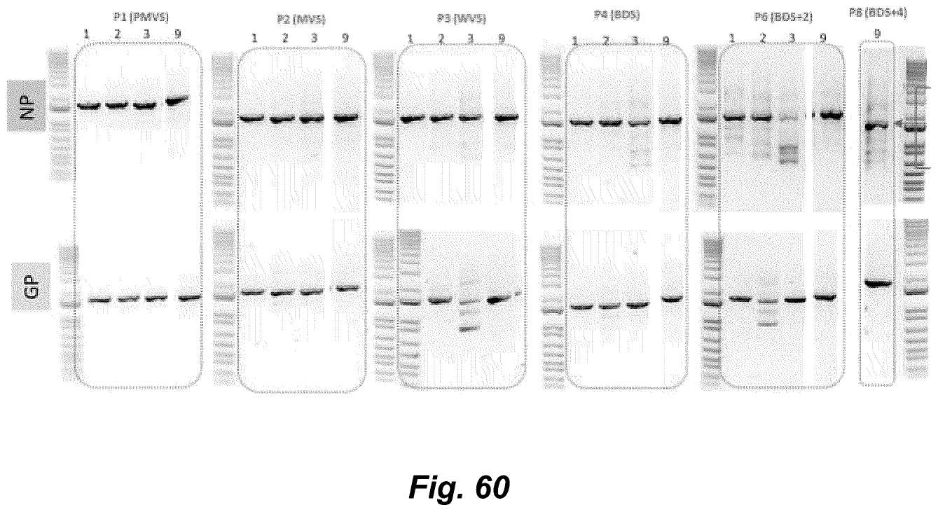

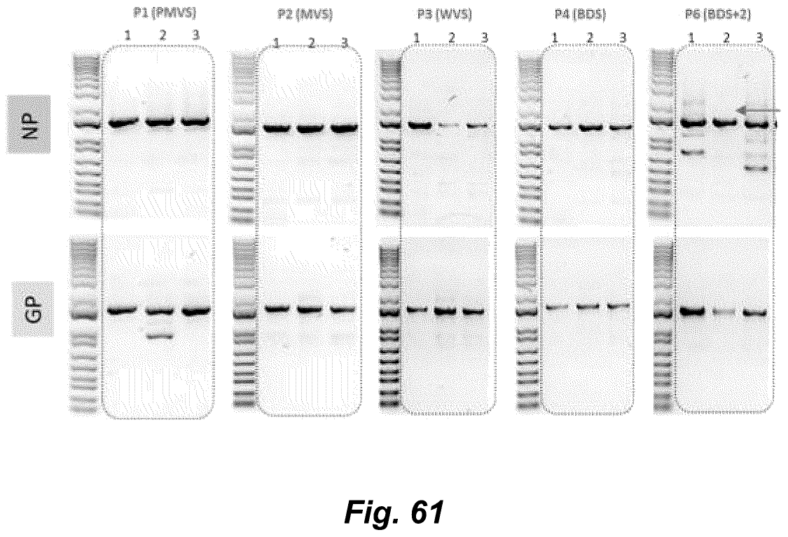

Evaluation of stable integration of HIV-1 Immunogen 2 transgene by serial passaging. To evaluate whether HIV-1 Immunogen 2 transgenes are stably encoded in replication attenuated arenavirus vectors over several passages, we analyzed cell culture supernatant and performed transgene PCR analysis. The passage level at which the majority (>50%) of the transgene specific band still shows the expected full-length size, was considered the last passage level with stable transgene insertion. Results are shown in Table 6 and FIGS. 58-61. Further, where applicable, supernatant samples harvested from each passaging step were applied to double-immunostaining (D1 staining), to determine the ratio of HIV-1 Gag and replication attenuated arenavirus-NP expression. A combination of both results allowed for a more accurate assessment of transgene stability of HIV-1 Immunogen 2 transgenes.

We found that replication attenuated-LCMV HIV antigen encoding vectors to stably encoded the transgenes greater than passage level 8, while replication attenuated-PICV HIV antigen encoding vectors stably encoded the transgenes to passage levels 6 and 8.

TABLE 6
Overview Table for Assessment of Transgene Genetic Stability

| | SEQ ID | | Transgene Stability |
|---|---|---|---|
| Vector Name | NOs: | Arenavirus | Through Passage (P) |
| TT1-HIV(C2)-GP1/PN1 | 84 and 88 | LCMV | >P8 |
| TT1-HIV(C2)-GP2/PN2 | 85 and 89 | LCMV | >P8 |
| TT2-HIV(C2)-GP1/PN1 | 82 and 86 | PICV | P8 |
| TT2-HIV(C2)-GP2/PN2 | 83 and 87 | PICV | >P6 |

P# indicates number of passages

Replication-attenuated-LCMV HIV antigen encoding vectors stably retained the transgenes encoding the fusion polypeptides SEQ ID NOs 84 and 88 (TT1-HIV (C2)-GP1/PN1) and SEQ ID NOs: 85 and 89 (TT1-HIV (C2)-GP2/PN2) greater than passage level 8. Replication-attenuated-PICV vector TT2-HIV (C2)-GP1/PN1 stably retained transgenes encoding fusion polypeptides SEQ ID NOs: 82 and 86 up to passage level 8 and replication attenuated-PICV vector TT2-HIV (C2)-GP2/PN2 stably retained transgenes encoding fusion polypeptides SEQ ID NOs: 83 and 87 greater than passage level 6, however.

### Example 13

### Vaccine and Immune Modulator Combinations

In non-human primates, administration of an adenoviral vector based vaccine encoding SIV immunogens in combination with checkpoint inhibitors such as αPD1 antibody has shown an increase in durability of vaccine induced T cell responses whereas combination with aCTLA4 antibody has demonstrated an increase in magnitude of T cell responses. See, Pan, et al., Front Immunol. (2018) 9:2415; and Loffredo, et al., Poster P55, Society for Immunotherapy of Cancer (SITC) 32nd Annual Meeting and Pre-Conference Programs; 2017 08-12 November; National Harbor, MD). FLT3L-FLT3 interaction leads to expansion and maturation of dendritic cells. Arenavirus based vectors show dendritic cell tropism (Flatz, et al., Nat Med (2010) 16 (3): 339-45). Therefore, we postulated that combining DC expansion with arenavirus vectors would enhance immunogenicity observed with the TT2/TT1 replication-attenuated PICV/LCMV arenavirus vector prime-boost scheme.

### Methods

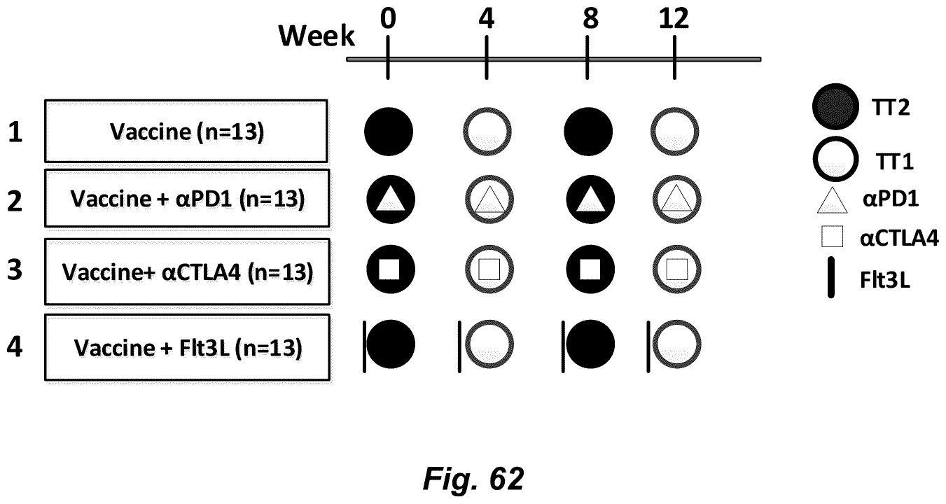

FIG. 62 provides a schematic of immunization schedule prime-boost with arenavirus vectors (TT2/TT1) in non-human primates (NHPs) in combination with immune modulators. Indian-origin healthy rhesus macaques were immunized via intramuscular (I.M.) route with the arenavirus vectors and via intravenous (I.V.) route with immune modulators with thirteen (13) NHPs in each group. The Gag and Env expressing vectors TT2 (replication-attenuated Pichinde (PICV)) and TT1 (replication-attenuated LCMV)) were administered in the left quadricep whereas the Pol expressing vectors (TT2 and TT1) were administered in the right quadricep. The doses administered are as below: 1×10⁶ replication competent virus particles (RCV) of TT2 Gag, Env and Pol1/Pol2 vectors, 4×10⁶ RCV of TT1 Gag, Env, and 2×10⁶ RCV of TT1 Pol1/Pol2.

The heterologous TT2/TT1 vaccine was administered every 4 weeks for a total of 4 doses either alone (Group 1, Vaccine) or in combination with checkpoint inhibitors (αPD1 antibody in group 2, or aCTLA4 antibody in group 3, 10 mg/kg each) administered immediately after the vaccine. The FLT3L-Fc FLT3 agonist was administered 1 week before the vaccine dose (Group 4, 0.3 mg/kg).

IFN-γ ELISpot assays were performed as described in Example 10.

### Results

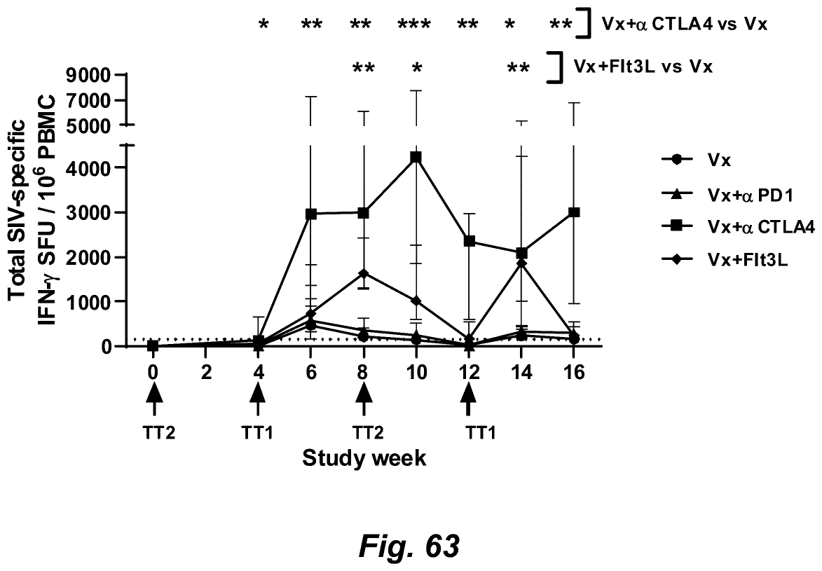

FIG. 63 shows the magnitude of SIV-specific IFN-γ responses as assessed by IFN-γ ELISpot in peripheral blood. Addition of αCTLA4 antibody with TT2/TT1 vaccine led to a robust and sustained increase in magnitude of SIV-specific responses compared to TT2/TT1 vaccine alone (p<0.05 at week 4, 6, 8, 10, 12, 14 and 16; Two-way ANOVA with Dunnett's multiple comparison test). Combination with the FLT3L-Fc FLT3 agonist also led to a significant increase in magnitude of SIV-specific response post boost (p<0.05 at weeks 8, 10 and 14; Two-way ANOVA with Dunnett's multiple comparison test). Administration of aPD1 antibody did not have any effect on the magnitude of vaccine-induced T cell response. Both checkpoint inhibitor aCTLA4 antibody and DC agonist FLT3L-Fc induced a significant increase in peak magnitude of T cell response consistent with the conclusion that vaccine-induced responses were augmented by these immune modulators.

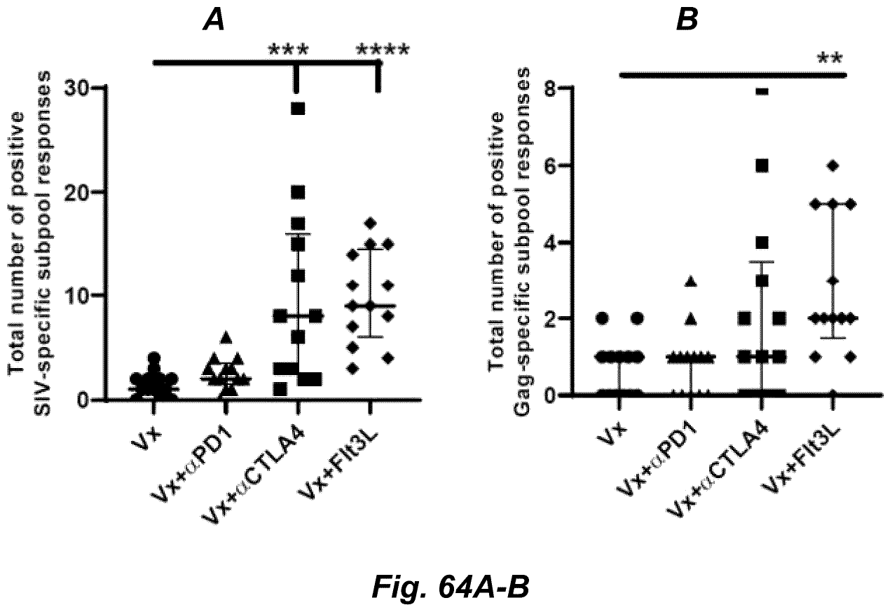

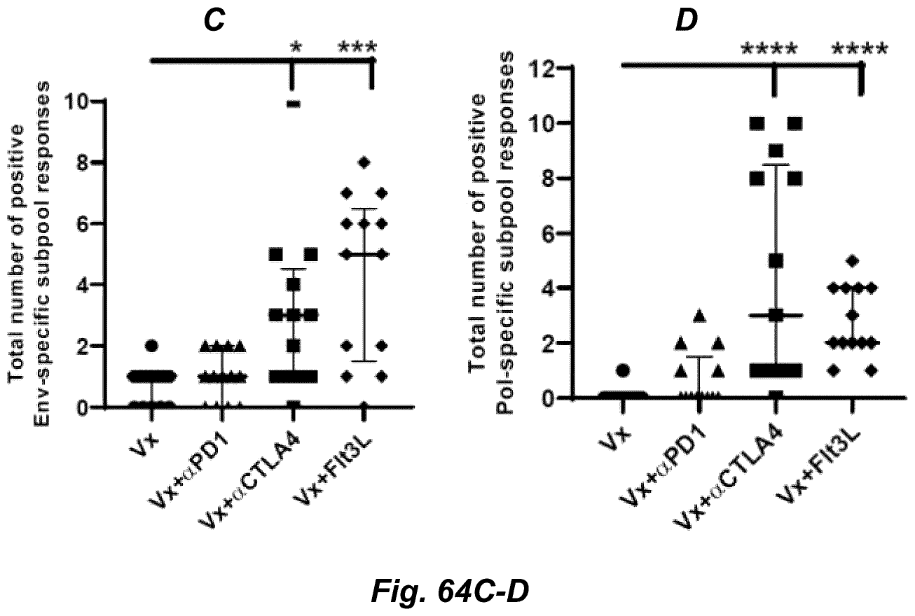

With respect to breadth, FIG. 64 shows that the highest total SIV-specific breadth at 2 weeks post last vaccine dose is observed in groups that received vaccine in combination with aCTLA4 antibody or FLT3L-Fc FLT3 agonist. Administration of TT2/TT1 vaccine in combination with FLT3L-Fc FLT3 agonist resulted in a significant increase in Gag, Env and Pol-specific breadth of responses. Administration of TT2/TT1 vaccine in combination with aCTLA4 antibody resulted in a significant increase in Env and Pol-specific breadth of responses. Taken together, these results are consistent with the conclusion that combination of heterologous TT2/TT1 vaccine with aCTLA4 antibody or FLT3L-Fc FLT3 agonist leads to a significant increase in breadth of SIV-specific T cell responses.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose

1-53. (canceled)

54. A polynucleotide comprising a nucleic acid sequence encoding one or more fusion polypeptides comprising or consisting of an amino acid sequence of any one of SEQ ID NOs: 82-89, or an amino acid sequence that is at least 90%, 91%, 92%, 93%, 94%, 95%, 96%, 97%, 98% or 99% identical to any one of SEQ ID NOs: 82-89, wherein the full-length of the fusion polypeptide comprises at least 350 amino acids and up to 385 amino acids, and wherein the fusion polypeptide is capable of inducing, promoting or stimulating an immune response in a human.

55-56. (canceled)

57. The polynucleotide of claim 54, comprising a nucleic acid sequence of any one of SEQ ID NOs: 150-159, or that is at least 85%, 86%, 87%, 88%, 89%, 90%, 91%, 92%, 93%, 94%, 95%, 96%, 97%, 98% or 99% identical to any one of SEQ ID NOs: 150-159.

58-59. (canceled)

60. An expression cassette, comprising a polynucleotide of claim 54 operably linked to one or more regulatory sequences.

61-62. (canceled)

63. A vector comprising one or more polynucleotides of claim 54.

64-68. (canceled)

69. The vector of claim 63, comprising one or more polynucleotides encoding the following first fusion polypeptide and second fusion polypeptide, optionally joined or connected by one or more linkers: SEQ ID NOs: 82 and 83, or fusion polypeptides that are at least 90%, 91%, 92%, 93%, 94%, 95%, 96%, 97%, 98% or 99% identical to any one of SEQ ID NOs: 82 and 83, respectively.

70. The vector of claim 63, comprising one or more polynucleotides encoding the following first fusion polypeptide and second fusion polypeptide, optionally joined or connected by one or more linkers: SEQ ID NOs: 84 and 85, or fusion polypeptides that are at least 90%, 91%, 92%, 93%, 94%, 95%, 96%, 97%, 98% or 99% identical to any one of SEQ ID NOs: 84 and 85, respectively.

71. The vector of claim 63, comprising one or more polynucleotides encoding the following first fusion polypeptide and second fusion polypeptide, optionally joined or connected by one or more linkers: SEQ ID NOs: 86 and 87, or fusion polypeptides that are at least 90%, 91%, 92%, 93%, 94%, 95%, 96%, 97%, 98% or 99% identical to any one of SEQ ID NOs: 86 and 87, respectively.

72. The vector of claim 63, comprising one or more polynucleotides encoding the following first fusion polypeptide and second fusion polypeptide, optionally joined or connected by one or more linkers: SEQ ID NOs: 88 and 89, or fusion polypeptides that are at least 90%, 91%, 92%, 93%, 94%, 95%, 96%, 97%, 98% or 99% identical to any one of SEQ ID NOs: 88 and 89, respectively.

73. The vector of claim 63, comprising one or more polynucleotides encoding the following first fusion polypeptide and second fusion polypeptide, optionally joined or connected by one or more linkers: SEQ ID NOs: 82 and 85, or fusion polypeptides that are at least 90%, 91%, 92%, 93%, 94%, 95%, 96%, 97%, 98% or 99% identical to any one of SEQ ID NOs: 82 and 85, respectively.

74. The vector of claim 63, comprising one or more polynucleotides encoding the following first fusion polypeptide and second fusion polypeptide, optionally joined or connected by one or more linkers: SEQ ID NOs: 82 and 86, or fusion polypeptides that are at least 90%, 91%, 92%, 93%, 94%, 95%, 96%, 97%, 98% or 99% identical to any one of SEQ ID NOs: 82 and 86, respectively.

75. The vector of claim 63, comprising one or more polynucleotides encoding the following first fusion polypeptide and second fusion polypeptide, optionally joined or connected by one or more linkers: SEQ ID NOs: 82 and 88, or fusion polypeptides that are at least 90%, 91%, 92%, 93%, 94%, 95%, 96%, 97%, 98% or 99% identical to any one of SEQ ID NOs: 82 and 88, respectively.

76. The vector of claim 63, comprising one or more polynucleotides encoding the following first fusion polypeptide and second fusion polypeptide, optionally joined or connected by one or more linkers: SEQ ID NOs: 83 and 87, or fusion polypeptides that are at least 90%, 91%, 92%, 93%, 94%, 95%, 96%, 97%, 98% or 99% identical to any one of SEQ ID NOs: 83 and 87, respectively.

77. The vector of claim 63, comprising one or more polynucleotides encoding the following first fusion polypeptide and second fusion polypeptide, optionally joined or connected by one or more linkers: SEQ ID NOs: 85 and 87, or fusion polypeptides that are at least 90%, 91%, 92%, 93%, 94%, 95%, 96%, 97%, 98% or 99% identical to any one of SEQ ID NOs: 85 and 87, respectively.

78. The vector of claim 63, comprising one or more polynucleotides encoding the following first fusion polypeptide and second fusion polypeptide, optionally joined or connected by one or more linkers: SEQ ID NOs: 88 and 87, or fusion polypeptides that are at least 90%, 91%, 92%, 93%, 94%, 95%, 96%, 97%, 98% or 99% identical to any one of SEQ ID NOs: 88 and 87, respectively.

79. The vector of claim 63, comprising one or more polynucleotides encoding the following first fusion polypeptide and second fusion polypeptide, optionally joined or connected by one or more linkers: SEQ ID NOs: 84 and 88, or fusion polypeptides that are at least 90%, 91%, 92%, 93%, 94%, 95%, 96%, 97%, 98% or 99% identical to any one of SEQ ID NOs: 84 and 88, respectively.

80. The vector of claim 63, comprising one or more polynucleotides encoding the following first fusion polypeptide and second fusion polypeptide, optionally joined or connected by one or more linkers: SEQ ID NOs: 85 and 89, or fusion polypeptides that are at least 90%, 91%, 92%, 93%, 94%, 95%, 96%, 97%, 98% or 99% identical to any one of SEQ ID NOs: 85 and 89, respectively.

81-104. (canceled)

105. The vector of claim 63, comprising the following first polynucleotide and second polynucleotide:
a) a first polynucleotide comprising SEQ ID NO: 150, or a polynucleotide that is at least 85%, 86%, 87%, 88%, 89%, 90%, 91%, 92%, 93%, 94%, 95%, 96%, 97%, 98% or 99% identical to SEQ ID NO: 150, respectively, and
b) a second polynucleotide comprising SEQ ID NO: 152, or a polynucleotide that is at least 85%, 86%, 87%, 88%, 89%, 90%, 91%, 92%, 93%, 94%, 95%, 96%, 97%, 98% or 99% identical to SEQ ID NO: 152.

106. The vector of claim 63, comprising the following first polynucleotide and second polynucleotide:
a) a first polynucleotide comprising SEQ ID NO: 151, or a polynucleotide that is at least 85%, 86%, 87%, 88%, 89%, 90%, 91%, 92%, 93%, 94%, 95%, 96%, 97%, 98% or 99% identical to SEQ ID NO: 151, respectively, and
b) a second polynucleotide comprising SEQ ID NO: 153, or a polynucleotide that is at least 85%, 86%, 87%, 88%, 89%, 90%, 91%, 92%, 93%, 94%, 95%, 96%, 97%, 98% or 99% identical to SEQ ID NO: 153.

107. The vector of claim 63, comprising the following first polynucleotide and second polynucleotide:
a) a first polynucleotide comprising SEQ ID NO: 154, or a polynucleotide that is at least 85%, 86%, 87%, 88%, 89%, 90%, 91%, 92%, 93%, 94%, 95%, 96%, 97%, 98% or 99% identical to SEQ ID NO: 154, respectively, and
b) a second polynucleotide comprising SEQ ID NO: 157, or a polynucleotide that is at least 85%, 86%, 87%, 88%, 89%, 90%, 91%, 92%, 93%, 94%, 95%, 96%, 97%, 98% or 99% identical to SEQ ID NO: 157.

108. The vector of claim 63, comprising the following first polynucleotide and second polynucleotide:
a) a first polynucleotide comprising SEQ ID NO: 155, or a polynucleotide that is at least 85%, 86%, 87%, 88%, 89%, 90%, 91%, 92%, 93%, 94%, 95%, 96%, 97%, 98% or 99% identical to SEQ ID NO: 155, respectively, and
b) a second polynucleotide comprising SEQ ID NO: 158, or a polynucleotide that is at least 85%, 86%, 87%, 88%, 89%, 90%, 91%, 92%, 93%, 94%, 95%, 96%, 97%, 98% or 99% identical to SEQ ID NO: 158.

109. The vector of claim 63, comprising the following first polynucleotide and second polynucleotide:
a) a first polynucleotide comprising SEQ ID NO: 156, or a polynucleotide that is at least 85%, 86%, 87%, 88%, 89%, 90%, 91%, 92%, 93%, 94%, 95%, 96%, 97%, 98% or 99% identical to SEQ ID NO: 156, respectively, and
b) a second polynucleotide comprising SEQ ID NO: 159, or a polynucleotide that is at least 85%, 86%, 87%, 88%, 89%, 90%, 91%, 92%, 93%, 94%, 95%, 96%, 97%, 98% or 99% identical to SEQ ID NO: 159.

110-127. (canceled)

128. The vector of claim 63, wherein the vector is a viral vector.

129-131. (canceled)

132. The vector of claim 63, wherein the viral vector is from a virus from a taxonomical family selected from Adenoviridae, Arenaviridae, Herpesviridae, Poxviridae, Flaviviridae, Rhabdoviridae, Togaviridae.

133. The vector of claim 63, wherein the viral vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

134-138. (canceled)

139. A host cell comprising one or more polynucleotides of claim 54.

140-215. (canceled)

216. A kit comprising one or more unitary doses of one or more vectors of claim 63.

217-311. (canceled)

312. A method for eliciting an immune response to human immunodeficiency virus (HIV) in a subject in need thereof, comprising administering to the subject one or more vectors of claim 63.

313. (canceled)

314. A method of treating or preventing human immunodeficiency virus (HIV) in a subject in need thereof, comprising administering to the subject one or more vectors of claim 63.

315-504. (canceled)


SEQUENCE LISTING (231 sequences)

SEQ ID NO: 1 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 2 - protein, 1003 aa, Human immunodeficiency virus 1
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 3 - protein, 206 aa, Human immunodeficiency virus 1
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 4 - protein, 53 aa, Human immunodeficiency virus 1
MAARASVLSGGELDRWEKIRLRPGGKKKYRLKHIVWASRELERFAVNPGLLET

SEQ ID NO: 5 - protein, 53 aa, Human immunodeficiency virus 1
MAARASILSGGKLDKWEKIRLRPGGRKKYKLKHLVWASRELERFALNPGLLET

SEQ ID NO: 6 - protein, 223 aa, Human immunodeficiency virus 1
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 7 - protein, 223 aa, Human immunodeficiency virus 1
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQ

SEQ ID NO: 8 - protein, 62 aa, Human immunodeficiency virus 1
FPQITLWQRPLVTIKIGGQLKEALLDTGADDTVLEEMNLPGRWKPKMIGGIGGFIKVRQYDQ

SEQ ID NO: 9 - protein, 62 aa, Human immunodeficiency virus 1
LPQITLWQRPIVTIKIGGQIKEALLDTGADDTVLEDMNLPGKWKPKMIGGIGGFIKVKQYDQ

SEQ ID NO: 10 - protein, 192 aa, Human immunodeficiency virus 1
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 11 - protein, 192 aa, Human immunodeficiency virus 1
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 12 - protein, 65 aa, Human immunodeficiency virus 1
WGFTTPDKKHQKEPPFLWMGYELHPDKWTVQPIVLPEKDSWTVNDIQKLVGKLNWASQIYPGIKV

SEQ ID NO: 13 - protein, 65 aa, Human immunodeficiency virus 1
WGLTTPDKKHQKDPPFLWMGYELHPDRWTVQPIELPEKESWTVNDIQKLIGKLNWASQIYAGIKV

SEQ ID NO: 14 - protein, 65 aa, Human immunodeficiency virus 1
PKFKLPIQKETWETWWTEYWQATWIPEWEFVNTPPLVKLWYQLEKEPIVGAETFYVDGAANRETK

SEQ ID NO: 15 - protein, 65 aa, Human immunodeficiency virus 1
PKFRLPIQKETWDTWWTDYWQATWIPEWEFTNTPPLVKLWYQLETEPIAGVETFYVDGASNRETK

SEQ ID NO: 16 - protein, 21 aa, Human immunodeficiency virus 1
KEPIVGAETFYVDGAANRETK

SEQ ID NO: 17 - protein, 21 aa, Human immunodeficiency virus 1
TEPIAGVETFYVDGASNRETK

SEQ ID NO: 18 - protein, 26 aa, Human immunodeficiency virus 1
KEKVYLAWVPAHKGIGGNEQVDKLVS

SEQ ID NO: 19 - protein, 26 aa, Human immunodeficiency virus 1
KEKIYLAWVPAHKGIGGNEQIDKLVS

SEQ ID NO: 20 - protein, 81 aa, Human immunodeficiency virus 1
VAKEIVASCDKCQLKGEAMHGQVDCSPGIWQLDCTHLEGKIILVAVHVASGYIEAEVIPAETGQETAYFLLKLAGRWPVKT

SEQ ID NO: 21 - protein, 81 aa, Human immunodeficiency virus 1
VAKEIVACCDKCQLKGEAIHGQVDCSPGVWQLDCTHLEGKVILVAVHVASGYIEAEIIPTETGQETAYFILKLAGRWPVTT

SEQ ID NO: 22 - protein, 69 aa, Human immunodeficiency virus 1
TVKAACWWAGIKQEFGIPYNPQSQGVVESMNKELKKIIGQVRDQAEHLKTAVQMAVFIHNFKRKGGIGG

SEQ ID NO: 23 - protein, 69 aa, Human immunodeficiency virus 1
AVKAACWWAGVKQEFGIPYNTQSQGVVESMNNELKKIIGQIRDQAEHLKTAVQMAVLIHNFKRKGGIGE

SEQ ID NO: 24 - protein, 81 aa, Human immunodeficiency virus 1
TVKAACWWAGIKQEFGIPYNPQSQGVVESMNKELKKIIGQVRDQAEHLKTAVQMAVFIHNFKRKGGIGGYSAGERIVDIIA

SEQ ID NO: 25 - protein, 81 aa, Human immunodeficiency virus 1
AVKAACWWAGVKQEFGIPYNTQSQGVVESMNNELKKIIGQIRDQAEHLKTAVQMAVLIHNFKRKGGIGEYSAGERIIDIIA

SEQ ID NO: 26 - protein, 72 aa, Human immunodeficiency virus 1
ITKIQNFRVYYRDSRDPLWKGPAKLLWKGEGAVVIQDNSDIKVVPRRKAKIIRDYGKQMAGDDCVASRQDED

SEQ ID NO: 27 - protein, 72 aa, Human immunodeficiency virus 1
ITKLQNFRVYYRDNRDPLWKGPARLLWKGEGAVVIQDNSEIKVVPRRKVKIIRDYGKRMAGDDCVAGRQDED

SEQ ID NO: 28 - protein, 13 aa, Human immunodeficiency virus 1
EEVGFPVKPQVPL

SEQ ID NO: 29 - protein, 13 aa, Human immunodeficiency virus 1
EEVGFPVRPQVPL

SEQ ID NO: 30 - protein, 36 aa, Human immunodeficiency virus 1
EEVGFPVKPQVPLRPMTFKGALDLSHFLREKGGLEG

SEQ ID NO: 31 - protein, 36 aa, Human immunodeficiency virus 1
EEVGFPVRPQVPLRPMTYKGALDLSHFLKEKGGLEG

SEQ ID NO: 32 - protein, 32 aa, Human immunodeficiency virus 1
TQGYFPDWQNYTPGPGTRYPLTFGWCFKLVPV

SEQ ID NO: 33 - protein, 32 aa, Human immunodeficiency virus 1
TQGFFPDWQNYTPGPGIRFPLTFGWCFKLVPL

SEQ ID NO: 34 - skipped

SEQ ID NO: 35 - protein, 92 aa, Human immunodeficiency virus 1
EGCRQILGQLQPSLQTGSEELRSLYNTVATLYCVHQRIEIKDTKEALDKIEEEQNKSKKKAQQAAADTGHSNQVSQNYPIVQNIQGQMVHQA

SEQ ID NO: 36 - protein, 131 aa, Human immunodeficiency virus 1
VTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 37 - protein, 55 aa, Human immunodeficiency virus 1
FFREDLAFLQGKAREFSSEQTRANSPTRRELQVWGRDNNSPSEAGADRQGTVSFN

SEQ ID NO: 38 - protein, 11 aa, Human immunodeficiency virus 1
ILIEICGHKAI

SEQ ID NO: 39 - protein, 46 aa, Human immunodeficiency virus 1
KILEPFRKQNPDIVIYQYMDDLYVGSDLEIGQHRTKIEELRQHLLR

SEQ ID NO: 40 - protein, 109 aa, Human immunodeficiency virus 1
QLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKT

SEQ ID NO: 41 - protein, 75 aa, Human immunodeficiency virus 1
GKAGYVTNRGRQKVVTLTDTTNQKTELQAIYLALQDSGLEVNIVTDSQYALGIIQAQPDQSESELVNQIIEQLIK

SEQ ID NO: 42 - protein, 38 aa, Human immunodeficiency virus 1
AGIRKVLFLDGIDKAQDEHEKYHSNWRAMASDFNLPPV

SEQ ID NO: 43 - protein, 12 aa, Human immunodeficiency virus 1
IHTDNGSNFTGA

SEQ ID NO: 44 - protein, 11 aa, Human immunodeficiency virus 1
TDIQTKELQKQ

SEQ ID NO: 45 - protein, 63 aa, Human immunodeficiency virus 1
MGGKWSKSSVIGWPTVRERMRRAEPAADRVGAASRDLEKHGAITSSNTAATNAACAWLEAQEE

SEQ ID NO: 46 - protein, 17 aa, Human immunodeficiency virus 1
LIHSQRRQDILDLWIYH

SEQ ID NO: 47 - protein, 57 aa, Human immunodeficiency virus 1
PDKIEEANKGENTSLLHPVSLHGMDDPEREVLEWRFDSRLAFHHVARELHPEYFKNC

SEQ ID NO: 48-58 - skipped (11 entries)

SEQ ID NO: 59 - protein, 4 aa, synthetic construct
  REGION 1..4: note = Description of Artificial Sequence: Synthetic peptide
GGGS

SEQ ID NO: 60 - protein, 4 aa, synthetic construct
  REGION 1..4: note = Description of Artificial Sequence: Synthetic peptide
RAKR

SEQ ID NO: 61 - protein, 4 aa, synthetic construct
  REGION 1..4: note = Description of Artificial Sequence: Synthetic peptide
REKR

SEQ ID NO: 62 - protein, 4 aa, synthetic construct
  REGION 1..4: note = Description of Artificial Sequence: Synthetic peptide
RRKR

SEQ ID NO: 63 - protein, 19 aa, Porcine teschovirus 1
ATNFSLLKQAGDVEENPGP

SEQ ID NO: 64 - protein, 24 aa, Foot-and-mouth disease virus
APVKQTLNFDLLKLAGDVESNPGP

SEQ ID NO: 65 - protein, 28 aa, synthetic construct
  REGION 1..28: note = Description of Artificial Sequence: Synthetic peptide
RAKRAPVKQTLNFDLLKLAGDVESNPGP

SEQ ID NO: 66 - protein, 20 aa, Equine rhinitis A virus
QCTNYALLKLAGDVESNPGP

SEQ ID NO: 67 - protein, 18 aa, Thosea asigna virus
EGRGSLLTCGDVEENPGP

SEQ ID NO: 68-69 - skipped (2 entries)

SEQ ID NO: 70 - protein, 337 aa, synthetic construct
  REGION 1..337: note = Description of Artificial Sequence: Synthetic polypeptide
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETAAAKEKVYLAWVPAHKGIGGNEQVDKLVSTQGYFPDWQNYTPGPGTRYPLTFGWCFKLVPV

SEQ ID NO: 71 - protein, 339 aa, synthetic construct
  REGION 1..339: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAAMAARASILSGGKLDKWEKIRLRPGGRKKYKLKHLVWASRELERFALNPGLLETTQGFFPDWQNYTPGPGIRFPLTFGWCFKLVPLLIKKEKIYLAWVPAHKGIGGNEQIDKLVS

SEQ ID NO: 72 - protein, 360 aa, synthetic construct
  REGION 1..360: note = Description of Artificial Sequence: Synthetic polypeptide
WGFTTPDKKHQKEPPFLWMGYELHPDKWTVQPIVLPEKDSWTVNDIQKLVGKLNWASQIYPGIKVAAQEEEEVGFPVKPQVPLRPMTFKGALDLSHFLREKGGLEGFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 73 - protein, 356 aa, synthetic construct
  REGION 1..356: note = Description of Artificial Sequence: Synthetic polypeptide
LPQITLWQRPIVTIKIGGQIKEALLDTGADDTVLEDMNLPGKWKPKMIGGIGGFIKVKQYDQEEVGFPVRPQVPLRPMTYKGALDLSHFLKEKGGLEGWGLTTPDKKHQKDPPFLWMGYELHPDRWTVQPIELPEKESWTVNDIQKLIGKLNWASQIYAGIKVKGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 74 - protein, 335 aa, synthetic construct
  REGION 1..335: note = Description of Artificial Sequence: Synthetic polypeptide
PKFKLPIQKETWETWWTEYWQATWIPEWEFVNTPPLVKLWYQLEKEPIVGAETFYVDGAANRETKITKIQNFRVYYRDSRDPLWKGPAKLLWKGEGAVVIQDNSDIKVVPRRKAKIIRDYGKQMAGDDCVASRQDEDVAKEIVASCDKCQLKGEAMHGQVDCSPGIWQLDCTHLEGKIILVAVHVASGYIEAEVIPAETGQETAYFLLKLAGRWPVKTEEVGFPVKPQVPLRPMTFKGALDLSHFLREKGGLEGTVKAACWWAGIKQEFGIPYNPQSQGVVESMNKELKKIIGQVRDQAEHLKTAVQMAVFIHNFKRKGGIGGYSAGERIVDIIA

SEQ ID NO: 75 - protein, 335 aa, synthetic construct
  REGION 1..335: note = Description of Artificial Sequence: Synthetic polypeptide
PKFRLPIQKETWDTWWTDYWQATWIPEWEFTNTPPLVKLWYQLETEPIAGVETFYVDGASNRETKEEVGFPVRPQVPLRPMTYKGALDLSHFLKEKGGLEGVAKEIVACCDKCQLKGEAIHGQVDCSPGVWQLDCTHLEGKVILVAVHVASGYIEAEIIPTETGQETAYFILKLAGRWPVTTITKLQNFRVYYRDNRDPLWKGPARLLWKGEGAVVIQDNSEIKVVPRRKVKIIRDYGKRMAGDDCVAGRQDEDAVKAACWWAGVKQEFGIPYNTQSQGVVESMNNELKKIIGQIRDQAEHLKTAVQMAVLIHNFKRKGGIGEYSAGERIIDIIA

SEQ ID NO: 76 - protein, 340 aa, synthetic construct
  REGION 1..340: note = Description of Artificial Sequence: Synthetic polypeptide
TQGYFPDWQNYTPGPGTRYPLTFGWCFKLVPVKEKVYLAWVPAHKGIGGNEQVDKLVSAAYMAARASVLSGGELDRWEKIRLRPGGKKKYRLKHIVWASRELERFAVNPGLLETSEGISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 77 - protein, 334 aa, synthetic construct
  REGION 1..334: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQTQGFFPDWQNYTPGPGIRFPLTFGWCFKLVPLMAARASILSGGKLDKWEKIRLRPGGRKKYKLKHLVWASRELERFALNPGLLETKEKIYLAWVPAHKGIGGNEQIDKLVS

SEQ ID NO: 78 - protein, 360 aa, synthetic construct
  REGION 1..360: note = Description of Artificial Sequence: Synthetic polypeptide
FPQITLWQRPLVTIKIGGQLKEALLDTGADDTVLEEMNLPGRWKPKMIGGIGGFIKVRQYDQAAYEEVGFPVKPQVPLRPMTFKGALDLSHFLREKGGLEGAAWGFTTPDKKHQKEPPFLWMGYELHPDKWTVQPIVLPEKDSWTVNDIQKLVGKLNWASQIYPGIKVGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 79 - protein, 358 aa, synthetic construct
  REGION 1..358: note = Description of Artificial Sequence: Synthetic polypeptide
WGLTTPDKKHQKDPPFLWMGYELHPDRWTVQPIELPEKESWTVNDIQKLIGKLNWASQIYAGIKVEEVGFPVRPQVPLRPMTYKGALDLSHFLKEKGGLEGLPQITLWQRPIVTIKIGGQIKEALLDTGADDTVLEDMNLPGKWKPKMIGGIGGFIKVKQYDQAAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 80 - protein, 342 aa, synthetic construct
  REGION 1..342: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDAAYEEEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKVAKEIVASCDKCQLKGEAMHGQVDCSPGIWQLDCTHLEGKIILVAVHVASGYIEAEVIPAETGQETAYFLLKLAGRWPVKTTVKAACWWAGIKQEFGIPYNPQSQGVVESMNKELKKIIGQVRDQAEHLKTAVQMAVFIHNFKRKGGIGGYSAGERIVDIIA

SEQ ID NO: 81 - protein, 335 aa, synthetic construct
  REGION 1..335: note = Description of Artificial Sequence: Synthetic polypeptide
EEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAVKAACWWAGVKQEFGIPYNTQSQGVVESMNNELKKIIGQIRDQAEHLKTAVQMAVLIHNFKRKGGIGEYSAGERIIDIIAVAKEIVACCDKCQLKGEAIHGQVDCSPGVWQLDCTHLEGKVILVAVHVASGYIEAEIIPTETGQETAYFILKLAGRWPVTT

SEQ ID NO: 82 - protein, 353 aa, synthetic construct
  REGION 1..353: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQKEPIVGAETFYVDGAANRETKAAKEKVYLAWVPAHKGIGGNEQVDKLVS

SEQ ID NO: 83 - protein, 351 aa, synthetic construct
  REGION 1..351: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQKEKIYLAWVPAHKGIGGNEQIDKLVSTEPIAGVETFYVDGASNRETKAVKAACWWAGVKQEFGIPYNTQSQGVVESMNNELKKIIGQIRDQAEHLKTAVQMAVLIHNFKRKGGIGEYSAGERIIDIIA

SEQ ID NO: 84 - protein, 358 aa, synthetic construct
  REGION 1..358: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAKEPIVGAETFYVDGAANRETKAAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAAKEKVYLAWVPAHKGIGGNEQVDKLVSL

SEQ ID NO: 85 - protein, 353 aa, synthetic construct
  REGION 1..353: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIATEPIAGVETFYVDGASNRETKAAKEKIYLAWVPAHKGIGGNEQIDKLVS

SEQ ID NO: 86 - protein, 361 aa, synthetic construct
  REGION 1..361: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDVAKEIVASCDKCQLKGEAMHGQVDCSPGIWQLDCTHLEGKIILVAVHVASGYIEAEVIPAETGQETAYFLLKLAGRWPVKTGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTQEEEEVGFPVKPQVPL

SEQ ID NO: 87 - protein, 364 aa, synthetic construct
  REGION 1..364: note = Description of Artificial Sequence: Synthetic polypeptide
VAKEIVACCDKCQLKGEAIHGQVDCSPGVWQLDCTHLEGKVILVAVHVASGYIEAEIIPTETGQETAYFILKLAGRWPVTTITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKILQEEEEVGFPVRPQVPL

SEQ ID NO: 88 - protein, 365 aa, synthetic construct
  REGION 1..365: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDAAAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTVAKEIVASCDKCQLKGEAMHGQVDCSPGIWQLDCTHLEGKIILVAVHVASGYIEAEVIPAETGQETAYFLLKLAGRWPVKTQEEEEVGFPVKPQVPL

SEQ ID NO: 89 - protein, 363 aa, synthetic construct
  REGION 1..363: note = Description of Artificial Sequence: Synthetic polypeptide
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTITKLQNFRVYYRDNRDPLWKGPARLLWKGEGAVVIQDNSEIKVVPRRKVKIIRDYGKRMAGDDCVAGRQDEDVAKEIVACCDKCQLKGEAIHGQVDCSPGVWQLDCTHLEGKVILVAVHVASGYIEAEIIPTETGQETAYFILKLAGRWPVTTAAQEEEEVGFPVRPQVPL

SEQ ID NO: 90 - protein, 391 aa, synthetic construct
  REGION 1..391: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVAKEIVASCDKCQLKGEAMHGQVDCSPGIWQLDCTHLEGKIILVAVHVASGYIEAEVIPAETGQETAYFLLKLAGRWPVKTQEEEEVGFPVKPQVPL

SEQ ID NO: 91 - protein, 393 aa, synthetic construct
  REGION 1..393: note = Description of Artificial Sequence: Synthetic polypeptide
AVKAACWWAGVKQEFGIPYNTQSQGVVESMNNELKKIIGQIRDQAEHLKTAVQMAVLIHNFKRKGGIGELSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVTVAKEIVACCDKCQLKGEAIHGQVDCSPGVWQLDCTHLEGKVILVAVHVASGYIEAEIIPTETGQETAYFILKLAGRWPVTTAAQEEEEVGFPVRPQVPL

SEQ ID NO: 92 - protein, 391 aa, synthetic construct
  REGION 1..391: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGVAKEIVASCDKCQLKGEAMHGQVDCSPGIWQLDCTHLEGKIILVAVHVASGYIEAEVIPAETGQETAYFLLKLAGRWPVKTAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQQEEEEVGFPVKPQVPL

SEQ ID NO: 93 - protein, 394 aa, synthetic construct
  REGION 1..394: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQPVVAKEIVACCDKCQLKGEAIHGQVDCSPGVWQLDCTHLEGKVILVAVHVASGYIEAEIIPTETGQETAYFILKLAGRWPVTTAAYAVKAACWWAGVKQEFGIPYNTQSQGVVESMNNELKKIIGQIRDQAEHLKTAVQMAVLIHNFKRKGGIGEQEEEEVGFPVRPQVPL

SEQ ID NO: 94 - protein, 499 aa, synthetic construct
  REGION 1..499: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPV

SEQ ID NO: 95 - protein, 501 aa, synthetic construct
  REGION 1..501: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVS

SEQ ID NO: 96 - protein, 494 aa, synthetic construct
  REGION 1..494: note = Description of Artificial Sequence: Synthetic polypeptide
FPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 97 - protein, 499 aa, synthetic construct
  REGION 1..499: note = Description of Artificial Sequence: Synthetic polypeptide
LPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 98 - protein, 504 aa, synthetic construct
  REGION 1..504: note = Description of Artificial Sequence: Synthetic polypeptide
VAKEIVASCDKCQLKGEAMHGQVDCSPGIWQLDCTHLEGKIILVAVHVASGYIEAEVIPAETGQETAYFLLKLAGRWPVKTTQGYFPDWQNYTPGPGTRYPLTFGWCFKLVPVTVKAACWWAGIKQEFGIPYNPQSQGVVESMNKELKKIIGQVRDQAEHLKTAVQMAVFIHNFKRKGGIGGYSAGERIVDIIAAAMAARASVLSGGELDRWEKIRLRPGGKKKYRLKHIVWASRELERFAVNPGLLETSEGISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAAKEKVYLAWVPAHKGIGGNEQVDKLVS

SEQ ID NO: 99 - protein, 499 aa, synthetic construct
  REGION 1..499: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIAKEKIYLAWVPAHKGIGGNEQIDKLVSAAYMAARASILSGGKLDKWEKIRLRPGGRKKYKLKHLVWASRELERFALNPGLLETTQGFFPDWQNYTPGPGIRFPLTFGWCFKLVPLVAKEIVACCDKCQLKGEAIHGQVDCSPGVWQLDCTHLEGKVILVAVHVASGYIEAEIIPTETGQETAYFILKLAGRWPVTT

SEQ ID NO: 100 - protein, 499 aa, synthetic construct
  REGION 1..499: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDAAYEEEEVGFPVKPQVPLRPMTFKGALDLSHFLREKGGLEGAAWGFTTPDKKHQKEPPFLWMGYELHPDKWTVQPIVLPEKDSWTVNDIQKLVGKLNWASQIYPGIKVPKFKLPIQKETWETWWTEYWQATWIPEWEFVNTPPLVKLWYQLEKEPIVGAETFYVDGAANRETKFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 101 - protein, 493 aa, synthetic construct
  REGION 1..493: note = Description of Artificial Sequence: Synthetic polypeptide
PKFRLPIQKETWDTWWTDYWQATWIPEWEFTNTPPLVKLWYQLETEPIAGVETFYVDGASNRETKEEVGFPVRPQVPLRPMTYKGALDLSHFLKEKGGLEGLPQITLWQRPIVTIKIGGQIKEALLDTGADDTVLEDMNLPGKWKPKMIGGIGGFIKVKQYDQITKLQNFRVYYRDNRDPLWKGPARLLWKGEGAVVIQDNSEIKVVPRRKVKIIRDYGKRMAGDDCVAGRQDEDWGLTTPDKKHQKDPPFLWMGYELHPDRWTVQPIELPEKESWTVNDIQKLIGKLNWASQIYAGIKVKGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 102-104 - skipped (3 entries)

SEQ ID NO: 105 - protein, 1028 aa, synthetic construct
  REGION 1..1028: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPVRAKRAPVKQTLNFDLLKLAGDVESNPGPLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVS

SEQ ID NO: 106 - protein, 1028 aa, synthetic construct
  REGION 1..1028: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVSRAKRAPVKQTLNFDLLKLAGDVESNPGPTVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPV

SEQ ID NO: 107 - protein, 1022 aa, synthetic construct
  REGION 1..1022: note = Description of Artificial Sequence: Synthetic polypeptide
FPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTRAKRAPVKQTLNFDLLKLAGDVESNPGPNLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 108 - protein, 1021 aa, synthetic construct
  REGION 1..1021: note = Description of Artificial Sequence: Synthetic polypeptide
LPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTRAKRAPVKQTLNFDLLKLAGDVESNPGPFPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 109 - protein, 1034 aa, synthetic construct
  REGION 1..1034: note = Description of Artificial Sequence: Synthetic polypeptide
VAKEIVASCDKCQLKGEAMHGQVDCSPGIWQLDCTHLEGKIILVAVHVASGYIEAEVIPAETGQETAYFLLKLAGRWPVKTTQGYFPDWQNYTPGPGTRYPLTFGWCFKLVPVTVKAACWWAGIKQEFGIPYNPQSQGVVESMNKELKKIIGQVRDQAEHLKTAVQMAVFIHNFKRKGGIGGYSAGERIVDIIAAAMAARASVLSGGELDRWEKIRLRPGGKKKYRLKHIVWASRELERFAVNPGLLETSEGISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAAKEKVYLAWVPAHKGIGGNEQVDKLVSRAKRAPVKQTLNFDLLKLAGDVESNPGPHQALSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIAKEKIYLAWVPAHKGIGGNEQIDKLVSAAYMAARASILSGGKLDKWEKIRLRPGGRKKYKLKHLVWASRELERFALNPGLLETTQGFFPDWQNYTPGPGIRFPLTFGWCFKLVPLVAKEIVACCDKCQLKGEAIHGQVDCSPGVWQLDCTHLEGKVILVAVHVASGYIEAEIIPTETGQETAYFILKLAGRWPVTT

SEQ ID NO: 110 - protein, 1031 aa, synthetic construct
  REGION 1..1031: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIAKEKIYLAWVPAHKGIGGNEQIDKLVSAAYMAARASILSGGKLDKWEKIRLRPGGRKKYKLKHLVWASRELERFALNPGLLETTQGFFPDWQNYTPGPGIRFPLTFGWCFKLVPLVAKEIVACCDKCQLKGEAIHGQVDCSPGVWQLDCTHLEGKVILVAVHVASGYIEAEIIPTETGQETAYFILKLAGRWPVTTRAKRAPVKQTLNFDLLKLAGDVESNPGPVAKEIVASCDKCQLKGEAMHGQVDCSPGIWQLDCTHLEGKIILVAVHVASGYIEAEVIPAETGQETAYFLLKLAGRWPVKTTQGYFPDWQNYTPGPGTRYPLTFGWCFKLVPVTVKAACWWAGIKQEFGIPYNPQSQGVVESMNKELKKIIGQVRDQAEHLKTAVQMAVFIHNFKRKGGIGGYSAGERIVDIIAAAMAARASVLSGGELDRWEKIRLRPGGKKKYRLKHIVWASRELERFAVNPGLLETSEGISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAAKEKVYLAWVPAHKGIGGNEQVDKLVS

SEQ ID NO: 111 - protein, 1020 aa, synthetic construct
  REGION 1..1020: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDAAYEEEEVGFPVKPQVPLRPMTFKGALDLSHFLREKGGLEGAAWGFTTPDKKHQKEPPFLWMGYELHPDKWTVQPIVLPEKDSWTVNDIQKLVGKLNWASQIYPGIKVPKFKLPIQKETWETWWTEYWQATWIPEWEFVNTPPLVKLWYQLEKEPIVGAETFYVDGAANRETKFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTRAKRAPVKQTLNFDLLKLAGDVESNPGPPKFRLPIQKETWDTWWTDYWQATWIPEWEFTNTPPLVKLWYQLETEPIAGVETFYVDGASNRETKEEVGFPVRPQVPLRPMTYKGALDLSHFLKEKGGLEGLPQITLWQRPIVTIKIGGQIKEALLDTGADDTVLEDMNLPGKWKPKMIGGIGGFIKVKQYDQITKLQNFRVYYRDNRDPLWKGPARLLWKGEGAVVIQDNSEIKVVPRRKVKIIRDYGKRMAGDDCVAGRQDEDWGLTTPDKKHQKDPPFLWMGYELHPDRWTVQPIELPEKESWTVNDIQKLIGKLNWASQIYAGIKVKGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 112 - protein, 1020 aa, synthetic construct
  REGION 1..1020: note = Description of Artificial Sequence: Synthetic polypeptide
PKFRLPIQKETWDTWWTDYWQATWIPEWEFTNTPPLVKLWYQLETEPIAGVETFYVDGASNRETKEEVGFPVRPQVPLRPMTYKGALDLSHFLKEKGGLEGLPQITLWQRPIVTIKIGGQIKEALLDTGADDTVLEDMNLPGKWKPKMIGGIGGFIKVKQYDQITKLQNFRVYYRDNRDPLWKGPARLLWKGEGAVVIQDNSEIKVVPRRKVKIIRDYGKRMAGDDCVAGRQDEDWGLTTPDKKHQKDPPFLWMGYELHPDRWTVQPIELPEKESWTVNDIQKLIGKLNWASQIYAGIKVKGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTRAKRAPVKQTLNFDLLKLAGDVESNPGPITKIQNFRVYYRDSRDPLWKGPAKLLWKGEGAVVIQDNSDIKVVPRRKAKIIRDYGKQMAGDDCVASRQDEDAAYEEEEVGFPVKPQVPLRPMTFKGALDLSHFLREKGGLEGAAWGFTTPDKKHQKEPPFLWMGYELHPDKWTVQPIVLPEKDSWTVNDIQKLVGKLNWASQIYPGIKVPKFKLPIQKETWETWWTEYWQATWIPEWEFVNTPPLVKLWYQLEKEPIVGAETFYVDGAANRETKFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 113-114 - skipped (2 entries)

SEQ ID NO: 115 - protein, 16 aa, unidentified
  REGION 1..16: note = Description of Unknown: CD74 sequence
MHRRRSRSCREDQKPV

SEQ ID NO: 116 - protein, 16 aa, Vesicular stomatitis virus
MKCLLYLAFLFIGVNC

SEQ ID NO: 117 - protein, 17 aa, unidentified
  REGION 1..17: note = Description of Unknown: albumin sequence
KWVTFISLLFLFSSAYS

SEQ ID NO: 118 - protein, 17 aa, unidentified
  REGION 1..17: note = Description of Unknown: calreticulin sequence
MLLSVPLLLGLLGLAVA

SEQ ID NO: 119 - protein, 18 aa, unidentified
  REGION 1..18: note = Description of Unknown: CSF2, GM-CSF sequence
MWLQSLLLLGTVACSISV

SEQ ID NO: 120 - protein, 21 aa, unidentified
  REGION 1..21: note = Description of Unknown: CXCL10, IP-10 sequence
MNQTAILICCLIFLTLSGIQG

SEQ ID NO: 121 - protein, 23 aa, unidentified
  REGION 1..23: note = Description of Unknown: PLAT, t-PA sequence
MDAMKRGLCCVLLLCGAVFVSAR

SEQ ID NO: 122 - protein, 31 aa, unidentified
  REGION 1..31: note = Description of Unknown: Beta-catenin sequence
MRKAAVSHWQQQSYLDSGIHSGATTTAPSLS

SEQ ID NO: 123 - protein, 76 aa, unidentified [sequence omitted]

SEQ ID NO: 124 - protein, 23 aa, unidentified
  REGION 1..23: note = Description of Unknown: CCL7, MCP-3 sequence
MKASAALLCLLLTAAAFSPQGLA

SEQ ID NO: 125 - protein, 384 aa, unidentified
  REGION 1..384: note = Description of Unknown: LAMP-1 N-terminal sequence
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTLLPDARDPAFKAANGSLRALQATVGNSYKCNAEEHVRVTKAFSVNIFKVWVQAFKVEGGQFGSVEECLLDENSLEDI

SEQ ID NO: 126 - protein, 39 aa, unidentified
  REGION 1..39: note = Description of Unknown: LAMP-1 C-terminal sequence
GSEFTLIPIAVGGALAGLVIVLIAYLVGRKRSHAGYQTI

SEQ ID NO: 127-129 - skipped (3 entries)

SEQ ID NO: 130 - DNA, 1497 bp, synthetic construct
  misc_feature 1..1497: note = Description of Artificial Sequence: Synthetic polynucleotide
  source 1..1497: mol_type = other DNA
acagtgaaggcagcttgttggtgggccggaattaaacaggagtttggcatcccttataatcctcagtctcagggagtggtggagtctatgaacaaggagctgaagaagatcatcggccaggtgagagatcaggcagaacatctgaagacagcagtgcagatggccgtgtttatccacaacttcaagaggaagggcggcattggaggatatagcgcaggagaaagaatcgtggacatcatcgccatctctcctagaacactgaacgcttgggtgaaagtggtggaggagaaagcctttagcccagaagtgatccctatgttctcagctctgtcagaaggagctacacctcaggatctgaacaccatgctgaataccgtgggaggacatcaggcagctatgcagatgctgaaggagacaattaacgaggaagcagccgagtgggatagactgcatccagtgcacgcaggacctattgctccaggacagatgagagagcctagaggaagcgatattgccggcacaacatctacactgcaggaacagatcggttggatgaccaacaatcctcctatcccagtgggcgaaatctacaaacgctggatcatcctgggcctgaataagatcgtgagaatgtacagccccacaagcatcctggatatcagacagggacctaaggaacctttcagggattacgtggaccggttctacaagacactgagagcagaacaggcatctcaggaggtgaagaattggatgaccgagacactgctggtgcagaacgctaatccagattgcaagaccattctgaaagctctgggaccagcagctacactggaagagatgatgacagcttgtcagggagtgggaggaccaggacataaagctagagtgctggcagaagctatgtctcagatggcagctagagcttcagtgctgtcaggaggagaactcgataggtgggagaagatcagactgagaccaggaggcaagaagaagtacagactgaagcacatcgtgtgggcttctagagaactggagagatttgccgtgaatccaggactcctggaaacacctccagtggtggctaaagagattgtggcttcttgcgataagtgccagctgaaaggagaggctatgcacggacaggtggattgttctccaggaatttggcagctggattgtacacacctggagggaaagattattctggtggcagtgcacgtggcatcaggatatattgaggccgaagtgattccagcagaaacaggacaggagacagcttactttctgctcaaactggcaggtcgctggccagtgaaaaccaaggagaaggtgtacctggcttgggtgccagctcataaaggaattggcggaaacgagcaggtggataaactggtgtctacacagggctacttcccagattggcagaattacacaccaggaccaggcacaagatatcctctgacattcggttggtgtttcaagctggtgcccgtg

SEQ ID NO: 131 - DNA, 1497 bp, synthetic construct
  misc_feature 1..1497: note = Description of Artificial Sequence: Synthetic polynucleotide
  source 1..1497: mol_type = other DNA
acagtgaaagcagcttgttggtgggcaggaatcaagcaggagtttggcatcccttacaatcctcagtctcagggagtggtggaatctatgaacaaggagctgaagaagatcatcggccaggtgagagatcaggcagaacatctgaagacagcagtgcagatggcagtgtttatccacaatttcaagagaaagggcggcattggcggatatagcgccggagagagaatcgtggatatcatcgccatctctcctagaacactgaacgcttgggtgaaagtggtggaagagaaagccttctctccagaggtgatccctatgtttagcgctctgtcagaaggagctacacctcaggatctgaataccatgctgaataccgtgggcggacatcaggcagctatgcagatgctgaaagagacaatcaacgaagaagcagccgagtgggatagactgcatccagtgcacgcaggacctattgctccaggacagatgagagaacctagaggatcagacattgccggaacaacatctacactgcaggagcagatcggttggatgacaaacaaccctccaatcccagtgggagagatctacaagagatggatcatcctgggcctgaataagatcgtgagaatgtacagccccacaagcatcctggatatcagacagggacctaaggagcctttcagagattacgtggacaggttctacaagaccctgagagcagaacaggcttctcaggaggtgaaaaattggatgaccgaaacactgctggtgcagaacgctaatcccgattgcaagaccatcctgaaagctctgggaccagcagctacactggaagagatgatgacagcttgtcagggagtgggaggaccaggacataaagctagagtgctggcagaagctatgtctcagatggcagctagagcttcagtgctgtcaggaggagaactcgatagatgggaaaagatcagactgagaccaggaggaaagaagaagtacaggctgaagcacatcgtctgggcttctagagaactggagagatttgccgtgaatccaggactcctggaaacacctccagtggtggctaaagagattgtggcttcttgcgacaagtgtcagctgaaaggagaggctatgcacggacaggtggattgttctccaggaatttggcagctggattgcacacatctggaaggaaagattattctggtggcagtgcacgtggcatctggatatatcgaggccgaggtgattccagccgaaacaggacaggaaacagcctactttctcctgaaactggcaggtaggtggccagtgaagacaaaggagaaggtgtacctggcttgggtgccagcccataaaggaattggaggcaatgagcaggtggataaactggtgtcaacacagggctacttcccagattggcagaattacaccccaggaccaggaacaagatatcctctgacattcggttggtgctttaaactggtgcccgtg

SEQ ID NO: 132 - DNA, 1503 bp, synthetic construct
  misc_feature 1..1503: note = Description of Artificial Sequence: Synthetic polynucleotide
  source 1..1503: mol_type = other DNA
ctgtctcctagaacactgaacgcttgggtgaaggtgatcgaagagaaggcctttagcccagaagtgatccctatgttcacagctctgtcagaaggagctacacctcatgacctgaacaccatgctgaataccatcggaggacatcaggcagctatgcagatgctgaaggataccatcaacgaagaagcagccgagtgggatagagtgcatccagtgcacgcaggaccagtggctccaggacagatgagagatcctagaggaagcgatattgccggctctacatctacactgcaggaacagatcgcttggatgaccaacaatcctcctatcccagtgggcgatatctacaaacgctggatcatcatgggcctgaacaagatcgtgaggatgtacagcccagtgtctatcctggatatcaagcagggccctaaagaacctttcagggattacgtggaccggttctacagaacactgagagcagaacaggcctcacaggatgtgaagaattggatgaccgagacactgctggtgcagaacagcaaccccgattgcaagaccattctgaaagctctgggaccaggagctacactggaagagatgatgtcagcttgtcagggagtgggaggaccatctcataaagctagagtgctggccgaagctatgtgtcaggtggctaaagagatcgtggcttgttgcgacaagtgtcagctgaaaggagaggctattcacggacaggtggattgttctccaggagtctggcagctggattgtacacacctggagggaaaggtgattctggtggcagtgcacgtggcatcaggatatattgaggccgagatcattcctacagaaacaggacaggagaccgcttacttcatcctgaaactggcaggtaggtggccagtgacaacaatggcagctagagcttctatcctgagcggaggaaaactcgacaagtgggagaagatcagactgagaccaggaggcagaaagaagtacaagctgaagcatctcgtctgggcttctagagagctggaaagattcgctctgaatccaggtctgctggaaacagcagcagcagtgaaagcagcttgttggtgggcaggagtgaaacaggaatttggcatcccttacaatacacagtctcagggagtggtggagagcatgaacaacgagctgaagaagatcatcggccagatcagagatcaggcagaacatctgaagacagcagtgcagatggcagtgctgatccacaacttcaagaggaagggcggaatcggagaatatagcgccggcgagagaattatcgatatcatcgccacacagggctttttcccagattggcagaactatacaccaggaccaggaatcaggttccctctgacattcggttggtgtttcaagctggtgcctctgctgatcaagaaggagaaaatctatctggcttgggtgccagctcacaaaggaattggcggaaacgagcagatcgataagctggtgtct

SEQ ID NO: 133 - DNA, 1503 bp, synthetic construct
  misc_feature 1..1503: note = Description of Artificial Sequence: Synthetic polynucleotide
  source 1..1503: mol_type = other DNA
ctgtctcctagaacactgaacgcttgggtgaaggtgattgaggagaaagccttcagcccagaagtgatccctatgtttacagccctgagcgaaggagctacacctcacgatctgaataccatgctgaatacaatcggcggacatcaggcagccatgcagatgctgaaggatacaatcaacgaagaagcagccgagtgggatagagtgcatccagtgcacgcaggaccagtggctccaggacagatgagagatcctagaggaagcgatatcgccggatctacatctacactgcaggaacagatcgcttggatgacaaataacccccctatcccagtgggagatatctataagcgctggatcatcatgggcctgaacaagatcgtgaggatgtacagcccagtgagcatcctggatatcaagcagggacctaaggagcctttcagagattacgtggacaggttctacagaaccctgagagcagaacaggcttctcaggacgtgaagaattggatgaccgaaacactgctggtgcagaatagcaaccccgattgcaagaccatcctgaaagctctgggaccaggagctacactggaagaaatgatgagcgcttgtcagggagtgggaggaccatctcataaggctagagtgctggcagaagctatgtgtcaggtggctaaggagattgtggcttgttgcgacaagtgtcagctgaaaggagaggctattcacggacaggtggattgttctccaggagtctggcagctggattgtacacatctggagggaaaagtgattctggtggcagtgcacgtggcatcaggatatattgaggccgaaatcatccctacagagacaggacaggagacagcctactttatcctgaaactggcaggcagatggccagtgacaacaatggcagctagagcttctatcctgagcggaggaaagctggataagtgggaaaagatcagactgagaccaggaggaaggaagaagtacaagctgaagcacctggtctgggcttctagagaactggaaagattcgccctgaatccaggtctgctggaaacagcagcagcagtgaaagcagcttgttggtgggcaggagtgaaacaggagttcggaatcccctacaacacacagtctcagggagtggtggaatctatgaacaacgagctgaagaagatcatcggccagatcagagaccaggccgaacatctgaagacagcagtgcagatggcagtgctgattcacaatttcaagagaaagggcggcatcggagagtatagcgccggagagagaatcatcgatatcatcgctacacagggcttcttccccgattggcagaattacaccccaggcccaggcattagattccctctgacattcggttggtgcttcaaactggtgcctctgctgatcaagaaggagaagatctacctggcttgggtgccagctcataaaggaatcggaggaaacgagcagatcgataagctggtgtct

SEQ ID NO: 134 - DNA, 1482 bp, synthetic construct
  misc_feature 1..1482: note = Description of Artificial Sequence: Synthetic polynucleotide
  source 1..1482: mol_type = other DNA
tttcctcagatcactctctggcagagaccactggtgacaatcaagatcggaggacagctgaaagaagctctgctggatacaggagcagacgatacagtgctggaagagatgaatctgccaggtcgctggaaacctaagatgattggaggcattggcggctttatcaaggtgagacagtacgaccaggaggaagtgggatttccagtgaaacctcaggtgcctctgagacctatgacatttaagggcgctctggacctgtctcactttctgagagagaagggaggactggaaggactgatccctaagttcaagctgcctatccagaaggagacttgggaaacttggtggacagagtattggcaggctacttggattcccgagtgggaatttgtgaacacacctcctctggtgaagctgtggtatcagctggaaaaggagcctattgtgggcgcagaaacattctacgtggacggagcagctaacagagaaactaagtggggattcaccaccccagataagaagcaccagaaggagccaccatttctctggatgggatacgaactgcacccagataagtggacagtccagcctattgtgctgccagaaaaggactcttggaccgtgaacgatatccagaagctggtgggaaagctgaattgggcttctcagatctacccaggaatcaaggtgatcaccaagatccagaacttcagggtgtactacagagacagcagagatcctctctggaagggaccagctaaactcctctggaaaggagaaggagcagtggtgatccaggataacagcgacatcaaggtggtgcctagaagaaaggccaagatcatcagggactacggcaaacagatggcaggagacgattgcgtggcttctagacaggacgaagacggaacagtcctggtgggacctacaccagtgaatatcatcggcagaaatctcctgacacagatcggttgtaccctgaacttccctatcagccctatcgagacagtgccagtgaaactgaagccaggaatggacggacctaaagtcaagcagtggcctctgacagaagagaagatcaaggccctggtggagatttgcacagagatggagaaggagggaaagatcagcaagatcggcccagagaatccttacaacaccccagtgttcgccatcaagaagaaggatagcaccaagtggagaaagctggtggatttcagggagctgaacaagagaacccaggacttttgggaagtgcagctgggcatcccccatccagcaggactgaagaagaagaagagcgtgacagtgctggacgtgggagacgcttattttagcgtgcctctggacaaggacttcagaaagtacaccgccttcaccatcccttctatcaacaacgagaccccaggcatcagataccagtataacgtgctgcctcagggttggaaaggatctccagcaatctttcagtctagcatgacc

SEQ ID NO: 135 - DNA, 1482 bp, synthetic construct
  misc_feature 1..1482: note = Description of Artificial Sequence: Synthetic polynucleotide
  source 1..1482: mol_type = other DNA
ttccctcagattactctctggcagaggccactggtgacaattaagatcggaggacagctgaaagaagctctgctggatacaggagcagacgatacagtgctggaggaaatgaacctgccaggtcgctggaaacctaaaatgatcggaggaatcggcggctttattaaggtgagacagtacgatcaggaggaagtgggatttccagtgaaacctcaggtgcctctgagacctatgacatttaagggcgctctggatctgagccactttctgagagagaaaggaggactggaaggactgatccctaagttcaagctgcccatccagaaggagacttgggaaacttggtggaccgagtattggcaggcaacttggattcccgagtgggaatttgtgaacacacctcctctggtgaagctgtggtatcagctggaaaaggagcctatcgtgggagccgaaacattttacgtggacggagcagccaacagagaaactaagtggggattcaccaccccagataagaagcaccagaaagagcctccctttctctggatgggatacgaactgcacccagataagtggacagtccagcctattgtgctgccagaaaaggactcttggacagtgaacgacatccagaagctggtgggaaagctgaattgggcctctcagatttacccaggaatcaaggtgatcaccaagatccagaacttcagggtgtactacagggatagcagagatcctctctggaagggaccagctaaactcctctggaaaggagaaggagcagtggtgatccaggataatagcgacatcaaggtggtgcctagaagaaaggctaagatcatccgggactacggcaaacagatggcaggagacgattgcgtggcttctagacaggatgaagacggaacagtcctggtgggacctacaccagtgaacatcatcggcagaaacctgctgacacagatcggttgtaccctgaacttccctatctctcctatcgaaacagtgccagtgaagctgaagccaggaatggacggacctaaagtcaagcagtggcctctgacagaagagaagatcaaggctctggtggagatttgcaccgaaatggagaaggagggcaagatcagcaagatcggaccagagaatccttacaataccccagtgttcgccatcaagaagaaggacagcaccaagtggagaaagctggtggatttcagggaactgaacaagaggacccaggacttttgggaagtgcagctgggcatcccccatccagcaggactgaagaagaagaagagcgtgacagtgctggacgtgggagacgcttattttagcgtgcctctggacaaggacttcagaaagtacaccgccttcaccatccctagcatcaataacgagaccccaggcatcagataccagtataacgtgctgccacagggctggaaaggatctccagcaatctttcagagctctatgaca

SEQ ID NO: 136 - DNA, 1497 bp, synthetic construct
  misc_feature 1..1497: note = Description of Artificial Sequence: Synthetic polynucleotide
  source 1..1497: mol_type = other DNA
ctgcctcagattaccctgtggcagagacctatcgtgaccatcaagatcggaggacagatcaaagaagctctgctggatacaggagcagacgatacagtgctggaggatatgaacctgccaggtaagtggaagcctaagatgatcggcggaattggcggctttatcaaggtcaagcagtacgatcagtggggactgacaacaccagacaagaagcaccagaaggacccccctttcctctggatgggatacgaactgcatccagataggtggacagtgcagccaattgagctgccagaaaaggagtcttggacagtgaacgacatccagaagctgatcggcaagctgaattgggcttctcagatctacgccggaattaaggtggcagctcaggaagaagaagaagtgggatttccagtgagacctcaggtgcctctgagacctatgacatacaagggagctctggatctgagccactttctgaaagagaagggaggactggagggaattaccaagctgcagaacttcagggtgtactacagggacaacagagatcctctgtggaaaggaccagctagactcctctggaaaggagaaggagcagtggtgattcaggacaatagcgagatcaaggtggtgcctagaagaaaggtgaagatcatccgggactacggcaaaagaatggcaggagacgattgcgtggcaggaagacaggacgaggaccccaaattcagactgcctatccagaaggagacttgggacacttggtggacagattattggcaggcaacttggattcccgagtgggaatttaccaatacccctcctctggtcaagctctggtatcagctggaaacagagcctatcgcaggagtggaaacattctacgtggacggagcctctaatagagagacaaaagccgcaggaacagtgctgattggacctacaccagtgaacatcatcgggagaaacctgctgacacagctgggttgtacactgaacttccctatcagccctatcgatacagtgccagtgaaactgaagccaggaatggacggacctagagtgaaacagtggcctctgacagaagagaagatcaaggccctgatcgagatttgtacagagatggagaaggagggcaagatctctagaattggcccagagaacccctacaatacccctatctttgccatcaagaagaaggacggcaccaagtggagaaagctggtggatttcagggagctgaacaagaagacccaggacttttgggaagtgcagctgggcatcccccaccctagcggactgaaaaagaagaagagcgtgaccgtgctggatattggagacgcctattttagcgtgccactggataaggagttcagaaagtacaccgcctttaccgtgccttctaccaataacgagacaccaggagtgagataccagtacaacgtgctgcctatgggttggaagggatcaccagccatctttcagtgtagcatgaca

SEQ ID NO: 137 - DNA, 1497 bp, synthetic construct
  misc_feature 1..1497: note = Description of Artificial Sequence: Synthetic polynucleotide
  source 1..1497: mol_type = other DNA
ctgcctcagattactctctggcagaggcctattgtgacaatcaagatcggcggacagatcaaagaagccctgctggatacaggagcagacgatacagtgctggaggatatgaacctgccaggcaagtggaaacctaagatgatcggaggaatcggcggatttatcaaggtgaagcagtacgatcagtggggactgacaacaccagataagaagcaccagaaggaccccccattcctgtggatgggatacgaactgcatccagataggtggacagtgcagccaatcgaactgccagaaaaggagtcttggaccgtgaacgacatccagaaactgatcggcaagctgaattgggccagccagatttacgccggaatcaaagtggcagctcaggaagaagaggaagtgggatttccagtgagacctcaggtgcctctgagacctatgacatacaaaggcgctctggatctgagccactttctgaaagagaagggaggactggagggaattacaaagctgcagaacttccgggtgtactacagagacaacagagaccctctctggaaaggaccagctagactcctctggaaaggagaaggagcagtggtgatccaggataatagcgagatcaaggtggtgcctaggagaaaggtgaagatcatcagggattacggcaaaagaatggccggagacgattgcgtggcaggaagacaggacgaagatcccaagttcagactgcctatccagaaggagacttgggacacttggtggaccgattattggcaggcaacttggattcccgagtgggaatttaccaacacacctcctctggtgaagctgtggtatcagctggaaacagagcctattgccggagtggaaacattctacgtggacggagccagcaacagagagacaaaagccgccggaacagtgctgattggacctacacccgtgaatatcatcggaagaaatctgctgacacagctgggttgtaccctgaatttccctatcagccccatcgatacagtgccagtgaaactgaagccaggaatggacggacctagagtcaaacagtggcctctgacagaagagaagatcaaggccctgatcgagatttgtaccgagatggagaaggagggaaagatcagcagaatcggcccagagaatccttacaacacccccatcttcgccatcaagaagaaagacggaaccaagtggagaaagctggtggatttcagggagctgaacaagaagacccaggacttttgggaagtgcagctgggcatcccccaccctagcggcctgaagaagaaaaagagcgtgacagtgctggacattggagacgcttatttcagcgtgccactggataaggagttcagaaagtacaccgcctttaccgtgccttctacaaacaacgagacaccaggcgtgagatatcagtacaacgtgctgcctatgggttggaaaggatctcccgccatctttcagtgtagcatgaca

SEQ ID NO: 138 - DNA, 1512 bp, synthetic construct
  misc_feature 1..1512: note = Description of Artificial Sequence: Synthetic polynucleotide
  source 1..1512: mol_type = other DNA
gtggccaaagaaattgtggcctcttgcgataagtgccagctgaaaggagaggctatgcacggacaggtggattgttctccaggaatttggcagctggattgtacacacctggagggaaagattattctggtggcagtgcacgtggcatcaggatatattgaggccgaagtgattccagcagaaacaggacaggagacagcttactttctgctcaaactggcaggtcgctggccagtgaagacaacacagggctactttcctgattggcagaattacacaccaggaccaggaacaagataccctctgacctttggttggtgcttcaaactggtgcccgtgacagtgaaagcagcttgttggtgggcaggaattaagcaggagttcggcatcccttacaatcctcagtctcagggagtggtggaatctatgaacaaggagctgaagaagatcatcggccaggtgagagatcaggcagaacatctgaagacagcagtgcagatggcagtgttcatccacaacttcaagcggaagggaggaattggaggatatagcgcaggagagagaatcgtggatatcattgccgccgctatggcagctagagccagcgtgctgagcggaggagaactcgatcgctgggaaaagatcagactgagaccaggaggcaagaagaagtacagactgaagcacatcgtctgggcttctagagaactggagagatttgccgtgaatccaggactgctggaaacaagcgagggcatttctcctagaaccctgaacgcttgggtgaaagtggtggaagaaaaagccttctctccagaggtgatccctatgtttagcgctctgtcagaaggagctacacctcaggatctgaacaccatgctgaacacagtgggaggacatcaggcagctatgcagatgctgaaggagacaattaacgaagaagccgccgagtgggatagactgcatccagtgcacgcaggacctattgctccaggacagatgagagagcctagaggaagcgatattgccggaacaacaagcacactgcaggaacagatcggttggatgaccaataatccccctattccagtgggcgagatctataagcgctggattatcctgggcctgaacaagatcgtgagaatgtacagccccacctctatcctggatatcagacagggccctaaggaacctttcagagactacgtggacaggttctacaagacactgagagcagaacaggcatctcaggaggtgaagaattggatgaccgagacactgctggtgcagaacgccaatccagattgcaagacaattctgaaagccctgggaccagcagctacactggaagagatgatgaccgcttgtcagggagtgggaggaccaggacataaagctagagtgctggcagaagctatgtctcaggcagcagctaaggagaaagtgtatctggcttgggtgccagcccataaaggaattggaggaaacgagcaggtggataaactggtgtct

SEQ ID NO: 139 - DNA, 1512 bp, synthetic construct
  misc_feature 1..1512: note = Description of Artificial Sequence: Synthetic polynucleotide
  source 1..1512: mol_type = other DNA
gtggctaaggaaattgtggcctcttgcgacaagtgtcagctgaaaggagaggctatgcacggacaggtggattgttctccaggaatttggcagctggattgcacacatctggaaggaaagattattctggtggcagtgcacgtggcatctggatatatcgaggccgaggtgattccagccgaaacaggacaggaaacagcctactttctcctgaaactggcaggtaggtggccagtgaagacaacacagggctacttcccagattggcagaattacaccccaggaccaggaacaagataccctctgacctttggttggtgcttcaagctcgtcccagtgacagtgaaagcagcttgttggtgggcaggaattaaacaggagttcggaatcccttacaatcctcagtctcagggagtggtggaaagcatgaacaaggagctgaagaagatcatcggacaggtgagagatcaggcagaacatctgaagacagcagtgcagatggcagtgttcatccacaacttcaagaggaagggcggaattggaggatatagcgccggagagagaatcgtggatatcattgcagcagctatggcagctagagcttcagtgctgtcaggaggagaactcgataggtgggagaagatcagactgagaccaggaggcaagaagaagtacagactgaagcacatcgtgtgggcttctagagaactggagagattcgcagtgaatccaggactgctggaaacaagcgagggaattagccctagaaccctgaatgcttgggtgaaagtggtggaagagaaggccttcagcccagaggtgatccctatgtttagcgctctgtcagaaggagctacacctcaggatctgaacaccatgctgaatacagtgggaggacatcaggcagctatgcagatgctgaaggagaccatcaacgaagaagcagccgagtgggatagactgcatccagtgcacgcaggacctattgctccaggacagatgagagaacctagaggaagcgatatcgccggaacaacatctacactgcaggaacagatcggttggatgaccaacaaccctcctattccagtgggcgagatttacaagcgctggattatcctgggcctgaataagatcgtgagaatgtacagccctaccagcattctggacatcagacagggacctaaggagccttttagagactacgtggacaggttctacaagaccctgagagcagaacaggcatctcaggaggtgaagaattggatgaccgagacactgctggtgcagaacgctaatcccgattgcaagaccatcctgaaagctctgggaccagcagctacactggaagagatgatgacagcttgtcagggagtgggaggaccaggacataaagctagagtgctggcagaagctatgtctcaggcagcagctaaagagaaagtgtatctggcttgggtgccagctcacaaaggaattggaggaaacgagcaggtggataaactggtgagc

SEQ ID NO: 140 - DNA, 1512 bp, synthetic construct
  misc_feature 1..1512: note = Description of Artificial Sequence: Synthetic polynucleotide
  source 1..1512: mol_type = other DNA
gtggccaaagagattgtggcctcctgtgacaagtgccagctgaaaggagaggcaatgcatggacaggtggattgttctccaggaatctggcagctggattgcacacacctggagggaaagatcatcctggtggcagtgcatgttgcatcaggatacattgaggcagaagtgattccagcagaaacaggacaggagactgcttactttctgctgaaactggcaggaaggtggccagtgaagacaacacagggttatttccctgattggcagaactacaccccaggccctggcacaagataccctctgacctttggttggtgcttcaaactggtccctgtgacagtgaaagcagcttgttggtgggcaggcatcaagcaggagtttggcatcccttacaaccctcagtctcagggagttgtggaatccatgaacaaggagctgaagaagatcattggtcaggtgagggatcaggcagaacatctgaagacagcagtgcagatggcagtgttcatccacaatttcaagaggaagggaggaattggaggatacagtgcaggagagagaattgtggacatcattgcagctgcaatggcagcaagagccagtgtgctcagtggaggagaacttgacaggtgggaaaagatcagactgagaccaggaggcaagaagaagtacagactgaagcacattgtctgggcttccagagaactggagagatttgctgtgaatccagggctgctggaaacaagtgagggcatttctcccagaactctgaatgcttgggtgaaggtggtggaagaaaaagccttctctccagaggtgatccccatgttcagtgcactgtctgaaggagccacacctcaggacctcaacaccatgctgaacacagtgggaggacatcaggcagccatgcagatgctgaaggagaccatcaatgaagaagctgcagagtgggacaggctgcatccagttcatgcaggaccaattgctcctggacagatgagagagcccagaggaagtgacattgctggcacaaccagcacactgcaggaacagattggttggatgaccaacaatccccccattccagtgggagagatctacaagaggtggatcatccttggcctgaacaagattgtgagaatgtacagccccacttcaatcctggacatcagacagggccccaaggaacctttcagagactatgtggacaggttctacaagacactgagagcagaacaggcctcacaggaggtgaagaattggatgactgagacactgctggtgcagaatgccaatccagattgcaagacaattctgaaagccctgggtccagcagccacactggaagagatgatgacagcttgccagggagtgggtggaccaggacacaaagcaagagtgctggcagaagcaatgtctcaggctgcagccaaggagaaagtttatctggcttgggtcccagcgcacaaaggaattggaggaaatgagcaggtggacaaacttgtgtcc

SEQ ID NO: 141 - DNA, 1497 bp, synthetic construct
  misc_feature 1..1497: note = Description of Artificial Sequence: Synthetic polynucleotide
  source 1..1497: mol_type = other DNA
ctgtctcctagaacactgaacgcttgggtgaaagtgatcgaggaaaaggcctttagcccagaagtgatccctatgtttaccgccctgtcagaaggagctacacctcacgatctgaacaccatgctgaacacaatcggaggacatcaggcagctatgcagatgctgaaggatacaatcaacgaagaagccgccgagtgggatagagtgcatccagtgcacgcaggaccagtggctccaggacagatgagagatcctagaggaagcgatatcgcaggatctacaagcacactgcaggaacagatcgcttggatgaccaataatccccctattccagtgggcgatatctacaagcgctggatcatcatgggcctgaacaagatcgtgaggatgtacagcccagtgagcatcctggatatcaagcagggacctaaggagcctttcagagattacgtggacaggttctacagaacactgagagccgaacaggcatctcaggacgtgaagaattggatgaccgagacactgctggtgcagaacagcaatcccgattgcaagacaatcctgaaagctctgggaccaggagctacactggaggaaatgatgagcgcttgtcagggagtgggaggaccatctcataaagctagagtgctggccgaagctatgtgtcaggcagtgaaagcagcttgttggtgggcaggagtgaaacaggagttcggcatcccttacaacacccagtctcagggagtggtggaatctatgaacaacgagctgaagaagatcatcggccagatcagagaccaggcagaacatctgaagacagcagtgcagatggcagtgctgattcacaacttcaagagaaagggcggcattggagagtatagcgccggagagagaattatcgatatcatcgccaaggagaagatctatctggcttgggtgccagctcataaaggaatcggaggaaacgagcagatcgataagctggtgtcagccgcctatatggcagctagagcttctattctgagcggaggaaagctcgacaagtgggaaaagatcaggctgagaccaggaggcagaaagaagtacaagctgaagcatctcgtctgggcttctagagaactggaaagattcgctctgaatccaggactgctggaaacaacccagggcttcttccccgattggcagaattacaccccaggaccaggaatcagattccctctgaccttcggttggtgtttcaagctggtgcctctggtggctaaagagattgtggcttgttgcgacaagtgtcagctgaaaggagaggctattcacggacaggtggattgttctccaggagtctggcagctggattgtacacatctggagggaaaagtgattctggtggcagtgcacgtggcatcaggatatattgaggccgaaatcatccctacagagacaggacaggagacagcctactttatcctgaagctggcaggaagatggccagtgacaaca

SEQ ID NO: 142 - DNA, 1497 bp, synthetic construct
  misc_feature 1..1497: note = Description of Artificial Sequence: Synthetic polynucleotide
  source 1..1497: mol_type = other DNA
ctgtctcctagaacactgaacgcttgggtgaaggtgattgaggagaaagccttcagcccagaagtgatccctatgtttacagccctgagcgaaggagctacacctcacgatctgaataccatgctgaatacaatcggcggacatcaggcagccatgcagatgctgaaggatacaatcaacgaagaagcagccgagtgggatagagtgcatccagtgcacgcaggaccagtggctccaggacagatgagagatcctagaggaagcgatatcgccggatctacatctacactgcaggaacagatcgcttggatgacaaataacccccctatcccagtgggagatatctataagcgctggatcatcatgggcctgaacaagatcgtgaggatgtacagcccagtgagcatcctggatatcaagcagggacctaaggagcctttcagagattacgtggacaggttctacagaaccctgagagcagaacaggcttctcaggacgtgaagaattggatgaccgaaacactgctggtgcagaatagcaaccccgattgcaagaccatcctgaaagctctgggaccaggagctacactggaagaaatgatgagcgcttgtcagggagtgggaggaccatctcataaggctagagtgctggcagaagctatgtgtcaggcagtgaaagcagcttgttggtgggcaggagtgaaacaggagtttggcatcccttacaatacccagtctcagggagtggtggaaagcatgaacaacgagctgaagaagatcatcggccagattagagatcaggccgaacatctgaaaacagcagtgcagatggccgtgctgatccacaatttcaagaggaagggcggaattggcgaatatagcgccggagagagaatcatcgacatcatcgccaaggagaagatctacctggcttgggtgccagctcataaaggaatcggaggaaacgagcagatcgataaactggtgtctgccgcttatatggcagctagagcttctatcctgtcaggaggaaagctcgataagtgggaaaagatcagactgagaccaggcggaagaaagaagtacaagctgaagcacctcgtctgggcttctagagaactggaaagattcgccctgaatccaggactgctggaaacaacacagggattcttccccgattggcagaactacacaccaggaccaggcatcagatttcctctgacctttggttggtgctttaaactggtgcctctggtggctaaggagattgtggcttgttgcgacaagtgtcagctgaaaggagaggctattcacggacaggtggattgttctccaggagtctggcagctggattgtacacacctggagggaaaagtgattctggtggcagtgcacgtggcatcaggatatatcgaggccgagatcatccctacagagacaggacaggaaaccgcctactttatcctgaaactggcaggtaggtggccagtgacaaca

SEQ ID NO: 143 - DNA, 1497 bp, synthetic construct
  misc_feature 1..1497: note = Description of Artificial Sequence: Synthetic polynucleotide
  source 1..1497: mol_type = other DNA
ctgagccccagaaccctcaatgcctgggtcaaagtgattgaggaaaaggccttcagcccagaggtgatccccatgttcacagccctcagtgagggggccaccccccatgacctgaacaccatgctcaacaccattgggggccaccaggctgccatgcagatgctgaaggacaccatcaatgaggaagctgctgagtgggacagagtccacccagtgcatgctggcccagtggccccaggccagatgagggaccccaggggctctgacattgctggcagcaccagcaccctgcaggagcagatagcctggatgaccaacaacccccccatccctgtgggagacatctacaaaagatggatcatcatgggcctcaacaagattgtcaggatgtactcccctgtgtccatcctggacatcaagcagggccccaaggaacccttcagggactatgtggacagattctacagaaccctgagagctgagcaggcctcccaggatgtgaaaaactggatgacagagaccctcctggtccagaacagcaacccagactgcaagaccatcctcaaggccttgggcccaggagccaccctggaagagatgatgagtgcctgccagggagttggaggccccagccacaaggccagagtgctggcagaggccatgtgccaggctgtgaaggctgcctgctggtgggcaggagtcaagcaggagtttggcatcccatacaacacccagagccagggtgtggtggaaagcatgaacaatgagctgaaaaagatcattggccagatcagagaccaggctgagcacctgaagacagcagtgcagatggctgtgctcatccacaacttcaagaggaaaggtggcataggggaatacagtgctggggagaggatcattgacatcattgccaaggagaagatctacctggcctgggtgcctgcccacaagggcattggtggcaatgagcagattgacaagctggtctcagcagcctacatggctgccagagcctccatcctctcagggggcaagctggacaagtgggagaaaatcagactgaggcctggtggcagaaagaagtacaagctgaagcacctggtgtgggcctccagggaactggaaagatttgccctgaaccctggcctgctggaaaccacccagggcttcttccctgactggcagaactacaccccaggcccaggcatcaggttccccctgacctttggctggtgcttcaagctggtgcccctggtggccaaggaaatagtggcctgctgtgacaagtgccagctgaaaggggaggccatccatggccaagtggactgcagccctggtgtgtggcagctggactgcacccacctggaaggcaaggtcatcctggttgcagtgcatgtggccagtggctacattgaggctgagatcatccccacagagacaggccaggagactgcctacttcatcctgaaacttgcaggcaggtggcctgtgaccacc

SEQ ID NO: 144 - DNA, 1497 bp, synthetic construct
  misc_feature 1..1497: note = Description of Artificial Sequence: Synthetic polynucleotide
  source 1..1497: mol_type = other DNA
atcaccaagatccagaacttcagggtgtactacagagacagcagagatcctctctggaagggaccagctaaactcctctggaaaggagaaggagcagtggtgatccaggataacagcgacatcaaggtggtgcctagaagaaaggccaagatcatcagggactacggcaaacagatggcaggagacgattgcgtggcttctagacaggacgaagacgcagcttacgaagaggaagaggtgggatttccagtgaaacctcaggtgcctctgagacctatgacattcaagggagctctggatctgtctcacttcctgagagaaaagggaggactggaaggagcagcttggggatttaccaccccagacaagaagcaccagaaggaaccaccattcctctggatgggatacgaactgcacccagataagtggacagtccagcctattgtgctgccagaaaaggactcttggaccgtgaacgatatccagaagctggtgggaaagctgaattgggcttctcagatctacccaggaatcaaggtgcccaagttcaagctgcctatccagaaggagacttgggaaacttggtggacagagtattggcaggctacttggattcccgagtgggaatttgtgaacacacctcctctggtgaagctgtggtatcagctggaaaaggagcctatcgtgggcgcagaaacattttacgtggacggagccgccaatagagaaaccaagtttcctcagatcactctctggcagagacctctggtgacaatcaagatcggcggacagctgaaagaggctctgctggatacaggagcagacgataccgtgctggaagaaatgaatctgccaggtaggtggaagcctaagatgattggcggaattggcggcttcatcaaggtgagacagtacgatcagggaacagtgctggtgggacctactccagtgaacatcatcggaaggaacctgctgacacagatcggctgtacactgaacttccctatcagccctatcgagacagtgccagtgaaactgaagccaggaatggacggacctaaagtcaaacagtggcctctgacagaggagaaaatcaaagccctggtggagatttgtaccgagatggagaaggagggcaagatttctaagatcggaccagagaacccctacaataccccagtgtttgccatcaagaagaaggacagcaccaagtggaggaagctggtggattttagggagctgaacaagaggacacaggacttttgggaagtgcagctgggcatcccccacccagccggactgaaaaagaagaagtcagtgacagtgctggacgtgggagatgcctattttagcgtgcctctggataaggacttcaggaagtacaccgccttcacaatccctagcatcaacaacgagaccccaggaatcagataccagtacaacgtgctgcctcagggttggaaaggaagcccagccatctttcagtctagcatgacc

SEQ ID NO: 145 - DNA, 1497 bp, synthetic construct
  misc_feature 1..1497: note = Description of Artificial Sequence: Synthetic polynucleotide
  source 1..1497: mol_type = other DNA
atcaccaagatccagaacttccgggtgtactacagggatagcagagatcctctctggaagggaccagctaaactcctctggaaaggagaaggagcagtggtgatccaggataatagcgacatcaaggtggtgcctagaagaaaggctaagatcatccgggactacggcaaacagatggcaggagacgattgcgtggcttctagacaggatgaagacgcagcctacgaagaagaggaagtgggatttccagtgaaacctcaggtgcctctgagacctatgacattcaagggagctctggacctgtctcactttctgagagagaagggaggactggaaggagcagcttggggatttaccacaccagataagaagcaccagaaggagccaccatttctctggatgggatacgaactgcacccagataagtggacagtccagcctattgtgctgccagaaaaggactcttggacagtgaacgacatccagaagctggtgggaaagctgaattgggcctctcagatttacccaggaatcaaggtgcccaagtttaagctgcctatccagaaggagacttgggaaacttggtggaccgagtattggcaggcaacttggattcccgagtgggaatttgtgaacacacctcctctggtgaagctgtggtatcagctggaaaaggagcctatcgtgggagccgaaacattttacgtggacggagcagccaacagagagacaaagttccctcagatcactctctggcagagaccactggtgacaattaagatcggaggacagctgaaagaagctctgctggatacaggagcagacgatacagtgctggaggaaatgaacctgccaggtcgctggaaacctaaaatgatcggcggcattggcggatttatcaaggtgaggcagtacgatcagggaacagtgctggtgggacctacacccgtgaatattatcggaaggaatctgctgacacagattggctgtaccctgaacttccctatcagccctatcgaaaccgtgccagtgaaactgaaaccaggaatggacggacctaaagtcaagcagtggcctctgacagaagagaagatcaaagccctggtggagatttgtaccgagatggagaaggagggaaagatcagcaagatcggcccagagaatccttacaacaccccagtgttcgccatcaagaagaaggatagcaccaagtggagaaagctggtggatttcagggagctgaacaagagaacccaggacttttgggaagtgcagctgggcatcccccaccctgccggcctgaagaagaagaaaagcgtgacagtgctggacgtgggagacgcttattttagcgtgcctctggacaaggacttcagaaagtacaccgccttcaccatcccttctatcaataacgagaccccaggcatcagataccagtataacgtgctgcctcagggttggaaaggaagcccagccatttttcagagcagcatgaca

SEQ ID NO: 146 - DNA, 1497 bp, synthetic construct
  misc_feature 1..1497: note = Description of Artificial Sequence: Synthetic polynucleotide
  source 1..1497: mol_type = other DNA
atcaccaagatccagaacttcagggtctactacagagacagcagagatccactctggaagggcccagccaaactcctttggaaaggagaaggagcagtggtgatccaggacaacagtgacatcaaggtggttcccagaagaaaggccaagatcatcagggactatggcaaacagatggcaggagatgactgtgtggcttccagacaggatgaagatgcagcttatgaagaggaagaggtgggattcccagtgaaaccccaggtgcctctgaggccaatgacattcaagggagctctggatctgtcccacttcctgagagaaaagggaggactggaaggagcagcttggggattcaccaccccagacaagaagcatcagaaggaacctcccttcctctggatgggttatgaactgcacccagacaagtggacagtccagcccattgtgctgccagaaaaggattcttggacagtgaatgacatccagaagctggtgggcaagctgaattgggcctcccagatctacccaggaatcaaggtgcccaagttcaagctccccatccagaaggagacttgggaaacttggtggacagagtattggcaggcaacttggattcctgagtgggaatttgtgaacaccccccctctggtgaagctgtggtatcagctggaaaaggagcccattgtgggggcagaaacattttatgtggatggagcagccaacagagaaaccaagtttccccagatcactctttggcagagaccactggtgacaatcaagattgggggacagctgaaagaggctctgctggacacaggagctgatgacacagtgctggaagaaatgaatctgccaggcaggtggaagcccaagatgattggaggcattgggggtttcatcaaggtgagacagtatgaccagggaactgtgctggtgggacccactccagtgaacatcattggcaggaacctgctgacacagattggctgcacactgaacttccccatcagccccattgagacagtcccagtgaaactgaagccaggaatggatggcccaaaagtcaaacaatggcccctgacagaggagaaaatcaaagctctggtggagatttgcacagagatggagaaggagggcaagatctctaagattggcccagagaacccatacaacactccagtgtttgcaatcaagaagaaggacagcaccaagtggaggaagctggtggacttcagggagctcaacaagaggacacaggacttttgggaagtgcagctgggcattccccacccagcaggactgaaaaagaagaagtcagtgacagtgctggatgtgggggatgcttatttcagtgtgcctctggacaaggacttcaggaagtacacagccttcacaatccccagcatcaacaatgagaccccaggaatcagatatcagtacaatgtgctgcctcagggttggaaaggaagcccagccattttccagtcgagcatgacc

SEQ ID NO: 147 - DNA, 1479 bp, synthetic construct
  misc_feature 1..1479: note = Description of Artificial Sequence: Synthetic polynucleotide
  source 1..1479: mol_type = other DNA
cctaaattcagactgcctatccagaaggagacttgggatacttggtggaccgattattggcaggcaacttggattcccgagtgggaatttaccaatacccctcctctggtgaagctgtggtatcagctggaaacagagcctattgccggagtggaaacattctacgtggacggagcctctaatagagagaccaaggaggaagtgggatttccagtgagacctcaggtgcctctgagacctatgacatacaagggagctctggacctgtctcactttctgaaggagaagggaggactggaaggactgcctcagattactctctggcagagacctatcgtgaccatcaagatcggaggacagatcaaagaagctctgctggatacaggagcagacgatacagtgctggaggatatgaacctgccaggtaagtggaagcctaagatgatcggcggaattggcggctttatcaaggtcaagcagtacgaccagatcaccaagctgcagaacttcagggtgtactacagggacaacagagaccccctctggaaaggaccagctagactcctctggaaaggagaaggagcagtggtgatccaggacaatagcgagatcaaggtggtgcctagaaggaaggtgaagatcatcagggactacggaaaaagaatggccggagacgattgcgtggcaggaagacaggacgaagattggggactgacaacaccagacaagaagcaccagaaggaccccccattcctctggatgggatacgaactgcatccagataggtggacagtgcagccaattgaactgccagagaaggagtcttggaccgtgaacgatatccagaagctgatcggcaagctgaattgggcttctcagatctacgcaggaatcaaggtgaagggcacagtgctgattggacctacaccagtgaacatcatcggcagaaacctcctgacacagctgggttgtacactgaatttccctatcagccctatcgatacagtgccagtgaagctgaaaccaggaatggacggacctagagtcaaacagtggcctctgacagaagagaagatcaaggccctgatcgagatttgcaccgagatggagaaggagggaaagatctctaggatcggcccagagaatccttacaatacccctatcttcgccatcaagaagaaggacggaaccaagtggagaaagctggtggatttcagggagctgaacaagaagacccaggacttttgggaggtgcagctgggcatcccccatccttcaggactgaagaagaagaagagcgtgacagtgctggatatcggagacgcttactttagcgtgccactggacaaggagttcagaaagtacaccgctttcaccgtgccttctaccaataacgagaccccaggagtgagataccagtataacgtgctgcccatgggttggaaaggatctccagcaatttttcagtgtagcatgaca

SEQ ID NO: 148 - DNA, 1479 bp, synthetic construct
  misc_feature 1..1479: note = Description of Artificial Sequence: Synthetic polynucleotide
  source 1..1479: mol_type = other DNA
cccaagttcaggctgcctatccagaaagagacttgggacacttggtggaccgattattggcaggcaacttggattcccgagtgggaatttaccaacacacctcctctggtgaagctgtggtatcagctggaaacagagcctattgccggagtggaaacattctacgtggacggagccagcaacagagagaccaaggaggaagtgggatttccagtgagacctcaggtgcctctgagacctatgacatacaagggagctctggatctgagccactttctgaaggagaaaggaggactggaaggactgcctcagattactctctggcagagacctatcgtgacaatcaagatcggcggacagatcaaggaagctctgctggatacaggagccgacgatacagtgctggaagatatgaacctgccaggcaagtggaaacctaagatgatcggaggcattggcggctttatcaaggtgaaacagtacgaccagatcaccaagctgcagaacttcagggtgtactacagggacaacagagaccccctctggaaaggaccagctagactgctgtggaaaggagaaggagcagtggtgatccaggataatagcgagatcaaggtggtgcctaggagaaaggtgaagatcatcagggactacggcaagagaatggcaggagacgattgcgtggcaggaagacaggacgaagattggggactgacaacaccagacaagaagcaccagaaggacccccctttcctctggatgggatacgaactgcatccagataggtggacagtgcagccaattgagctgccagaaaaggagtcttggacagtgaacgacatccagaagctgatcggcaagctgaattgggcttctcagatctacgccggaattaaggtgaagggaacagtgctgattggacctacaccagtgaacatcatcggaagaaacctgctgacacagctgggttgtacactgaacttccctatcagccctatcgataccgtgccagtgaaactgaaaccaggaatggacggacctagagtcaagcagtggcctctgacagaagagaagatcaaggccctgatcgagatttgcaccgagatggagaaggagggaaagatcagcagaattggccccgagaatccttacaacacccctatcttcgccatcaagaagaaggacggaactaagtggagaaagctggtggacttcagagagctgaacaagaagacccaggacttttgggaagtgcagctgggcatcccccatccttcaggactgaagaagaagaagagcgtgacagtgctggacatcggagacgcttattttagcgtgcctctggataaggagttccggaaatacaccgcctttaccgtgccttctaccaataacgagacaccaggagtgaggtaccagtataacgtgctgccaatgggctggaaaggatctccagcaatctttcagtgtagcatgaca

SEQ ID NO: 149 - DNA, 1479 bp, synthetic construct
  misc_feature 1..1479: note = Description of Artificial Sequence: Synthetic polynucleotide
  source 1..1479: mol_type = other DNA
cccaagttcaggctgcccatccagaaagaaacctgggacacctggtggactgactactggcaggccacctggatcccagagtgggagttcaccaacacccctcccctggtgaagctgtggtaccagctggaaactgagcccatagcaggagtggaaaccttttatgtggatggagccagcaacagggaaaccaaggaagaggtgggcttcccagtgaggccccaggtgcccctgagacccatgacctacaagggagccctggatctgagccacttcctgaaagagaaggggggcctggaaggcctgccccagatcaccctgtggcagagacccattgtgaccatcaagattgggggccagatcaaagaggccctcctggacactggagctgatgacacagtcctggaagacatgaacctcccaggaaagtggaagcccaagatgattgggggcattggaggcttcatcaaggtcaagcagtatgaccagatcaccaagctccagaatttcagagtctactacagagacaacagagaccccctgtggaaaggcccagccagacttctctggaagggggagggagcagtggtgatccaagacaactctgagatcaaagtggtccccaggagaaaggtgaagatcatcagggactatggcaaaaggatggcaggggatgactgtgtggcaggcagacaggatgaggactggggcctgaccacccctgacaagaagcaccagaaggacccccccttcctgtggatgggctatgagctgcacccagacagatggactgtccaacccattgagctgcctgagaaggagagctggacagtcaatgacatccagaagctgattgggaagctgaattgggcctcccagatctatgcaggcatcaaggtcaagggcactgtcctgattggccccacccctgtgaacatcataggcagaaacctgctgacccagctgggctgcacactgaacttccccatcagccccattgacacagtgcctgtgaagctcaagcctggcatggatggccccagagtgaaacagtggcccttgacagaggaaaaaatcaaggccctgattgagatctgcactgagatggaaaaagagggcaagatctcaagaattggccctgagaacccctacaacacccccatttttgccatcaagaaaaaggatggcacaaagtggagaaagctggtggacttcagagagctcaacaagaagacccaggacttctgggaggtgcagctgggcatcccccacccctctggcctcaagaagaagaaaagtgtgactgtgctggacattggtgatgcctacttcagtgtccccctggacaaggaattcaggaagtacacagccttcacagtgcccagcaccaacaatgagacccctggtgtcaggtaccagtacaatgtgctgcccatgggctggaagggcagcccagccatcttccagtgcagcatgacc

SEQ ID NO: 150 - DNA, 1059 bp, synthetic construct
  misc_feature 1..1059: note = Description of Artificial Sequence: Synthetic polynucleotide
  source 1..1059: mol_type = other DNA
actgtaaaggccgcctgttggtgggctgggatcaaacaggagttcggaatcccatacaatccccaaagccagggagtagtggaatccatgaataaggagctcaagaagatcattgggcaagtcagggaccaggctgagcacctcaagacggccgttcaaatggcggtatttatccataactttaaacgcaaaggcggtattggcggttactccgcaggtgagcggatagttgacattatcgcaataagccctaggacgcttaacgcttgggtcaaggtagtggaggaaaaagccttctcaccggaggtcatccctatgtttagcgccctgagcgaaggagcgacgccacaagacttgaacaccatgcttaatacggtggggggtcaccaagctgcaatgcaaatgttgaaggaaacgataaacgaagaggccgccgaatgggacaggttgcatcccgttcatgcaggtcccatagctcccggtcaaatgagggaaccacgcggatcagacatcgcgggaacgacgtccacgttgcaggaacaaattggatggatgaccaataaccctcctatcccagtcggtgaaatctataagaggtggatcatcctcggcctcaataaaatagtgaggatgtattctccaactagcattctggatatacgccaaggccctaaagaaccatttcgcgattacgtagaccgattttacaagacactccgggctgagcaggcctcccaagaggtaaagaattggatgaccgaaacgttgctggtgcagaatgccaaccccgactgtaagaccattttgaaggcgcttgggccagcagcaacactggaagagatgatgactgcgtgtcaaggtgtaggtggccccggccacaaagccagagtgttggccgaggcaatgagccaaaaagaacccattgtaggagctgagaccttttacgttgatggcgcagctaacagagaaactaaggcggcaaaagagaaagtttatcttgcatgggtgcctgcgcacaaaggaatcggcggaaatgagcaagttgataaactcgtaagt

SEQ ID NO: 151 - DNA, 1074 bp, synthetic construct
  misc_feature 1..1074: note = Description of Artificial Sequence: Synthetic polynucleotide
  source 1..1074: mol_type = other DNA
accgtgaaggcggcgtgttggtgggcagggataaagcaagaatttggcataccttataacccgcagagtcagggagttgtcgaatccatgaataaggagcttaagaagatcatcggtcaggtgcgcgatcaggcagagcacctcaaaactgcggtccaaatggccgtgttcatccacaacttcaagcgcaaaggcggaatcggtggatacagtgctggggaaaggatcgtcgatattatcgccaaagagccaatcgtgggagcggaaacattctacgtagacggtgcggccaacagggagacaaaagccgctgcgatctcaccccgaacccttaatgcttgggtgaaggtggtggaagaaaaagccttcagtcccgaagttatcccgatgttctccgccctcagtgaaggtgcaacgccgcaggaccttaatactatgttgaacaccgttggtggtcatcaggccgccatgcagatgcttaaagaaaccattaacgaagaagccgcagaatgggacaggttgcacccagtccacgctgggcccatcgcgcctgggcagatgcgagagccacgagggtccgatatcgcgggcacaacaagcactttgcaagaacagattgggtggatgactaataacccgccgatacctgttggggaaatatataaacgctggataattctgggactcaataagatagtgagaatgtactcccctacatccattcttgatatacgacaaggtccaaaagaaccctttcgcgactacgtggatagattttataagaccctcagggccgaacaggcaagtcaggaggtcaagaactggatgacggagacgcttcttgttcagaatgcaaatcccgattgcaaaactatcttgaaagcgctcggaccagcagcgacgctggaagaaatgatgaccgcctgccagggcgtaggcggcccaggtcataaagcaagggtattggcagaagcgatgagtcaagcagcggcaaaggagaaagtatatcttgcgtgggtaccagcgcacaaagggataggtggaaacgagcaagttgataagctggtctccctt

SEQ ID NO: 152 - DNA, 1083 bp, synthetic construct
  misc_feature 1..1083: note = Description of Artificial Sequence: Synthetic polynucleotide
  source 1..1083: mol_type = other DNA
ataacgaaaattcagaactttcgagtgtactacagagatagtagagacccgctgtggaagggcccagctaaactcctttggaaaggtgaaggggctgtggtcatacaggataactcagacataaaggtcgtcccaaggagaaaagccaaaataattagagactacggcaagcagatggcaggggatgactgtgttgcgagccggcaggatgaggatgtcgcgaaggaaatcgtggcgagttgtgataaatgtcaactgaagggtgaggcaatgcacggccaagtagattgcagtccaggtatctggcaactcgattgcacccacctggagggtaagattatcctggtggctgtgcatgttgcatccggctacatcgaagctgaagtgattccggctgaaacggggcaggaaaccgcctacttcctgttgaagttggctggtcgatggccagtcaagaccggtacagtactcgttggcccgacgccagtgaatatcataggtcggaacctgctgacacaaatcgggtgcactcttaattttccgatttcacctatcgaaaccgttccagtaaaactcaagcctgggatggatggcccgaaggttaagcaatggccactgaccgaagagaaaatcaaagcgctcgtggagatatgtactgaaatggagaaagaaggaaaaatctctaaaatcgggccagaaaatccctataatactccggtatttgctatcaaaaaaaaagactcaaccaagtggcgaaagctcgttgacttccgagagttgaataaaaggacccaggatttttgggaggttcagctgggcataccgcaccccgctggcttgaaaaaaaagaagtctgttaccgtcctggatgtgggcgacgcctacttcagtgtacctcttgacaaagactttagaaagtatactgctttcacgatcccgagtataaacaacgagactccaggaattaggtaccagtacaatgtattgccgcagggatggaagggatcacccgcaatcttccaatctagtatgacgcaagaggaggaggaagtaggtttcccagtcaaaccacaagtgccgctc

SEQ ID NO: 153 - DNA, 1095 bp, synthetic construct
  misc_feature 1..1095: note = Description of Artificial Sequence: Synthetic polynucleotide
  source 1..1095: mol_type = other DNA
attactaaaatacaaaacttccgagtatattatagggatagccgggaccctctctggaaagggcccgcgaaactgctttggaagggcgagggcgctgttgtcatacaggacaatagcgatataaaagtagtcccgcgacgcaaagcaaaaataataagagattatggaaaacaaatggcgggcgacgattgtgtcgcctcacggcaggacgaggatgctgcagccatcggaacagtactcgtggggccaactcccgtcaacataataggacgaaatcttctgactcaaataggttgcacgctcaacttcccaattagccctatagagacagtgcccgtaaagttgaagcctggcatggacggcccaaaagtaaagcagtggccccttactgaagagaagatcaaggccctggtggagatctgtacagagatggagaaagaagggaaaatctccaaaatcggtccagaaaacccatacaacacacctgtttttgcgataaaaaaaaaggattccaccaagtggaggaaactggtggattttcgagagttgaataagcgaacccaggacttctgggaagtacagcttgggataccacatccagcgggtctcaagaaaaagaaatcagttacggtcctcgatgttggcgacgcgtattttagtgttcctctcgataaagatttccgaaaatatacggcattcaccatacccagtattaacaacgagacaccgggaatcaggtatcagtataatgtacttccacaaggttggaagggaagccccgcgatttttcagagctcaatgacggttgccaaggagattgtagctagctgtgacaaatgtcagctgaagggcgaagcgatgcacggccaggtagattgcagccccggtatttggcagctcgactgtacacatttggaaggaaaaattatacttgtggctgtccacgttgcaagtggatacatagaggcggaggtcattcccgcagaaactgggcaagaaactgcttactttctcttgaagttggccggtaggtggccagtgaaaacccaagaggaagaggaggttggttttcccgtcaaacctcaagtccccctc

SEQ ID NO: 154 - DNA, 1053 bp, synthetic construct
  misc_feature 1..1053: note = Description of Artificial Sequence: Synthetic polynucleotide
  source 1..1053: mol_type = other DNA
ctgagccccaggaccctgaatgcatgggtcaaagtgattgaagagaaggctttctctcctgaagtcatcccaatgttcacagcactttctgagggggccacaccacatgatttgaacaccatgctcaacaccattggtggtcatcaggctgctatgcagatgctcaaggacaccatcaatgaggaagcagcagagtgggacagagtccacccagtccatgcagggcctgttgcaccagggcaaatgagggaccccagaggctcagacattgcaggttccacatcaacccttcaagaacaaattgcctggatgacaaacaatcccccaatcccagtgggggacatctacaagagatggatcatcatgggcctgaacaaaattgtgaggatgtacagccctgtgtcaattctggacatcaagcaaggcccaaaagaacctttcagggactatgttgacaggttttacaggaccctcagggcagaacaggcctcccaggatgtgaagaattggatgacagagacattgcttgttcagaacagcaacccagactgcaagaccattctgaaagccctgggtccaggagcaaccctggaagagatgatgtcagcctgtcagggggtgggggggccatcacacaaagccagggtgttggctgaggccatgtgccagaaagaaaaaatctatctggcatgggtgccagcccacaaggggattggaggcaatgagcaaattgacaagctggtgtcaactgaacccattgctggggtagagactttttatgtggatggtgccagcaacagggaaaccaaggctgtgaaggctgcttgttggtgggctggagtcaagcaggagtttggcattccctacaacacccaaagccaaggtgttgttgaatccatgaacaatgagttgaaaaagatcataggccagatcagggatcaagctgagcatctgaaaactgcagttcagatggcagtcctgattcacaatttcaagagaaagggagggataggagagtacagtgcaggggaaaggatcattgacatcattgca

SEQ ID NO: 155 - DNA, 1053 bp, synthetic construct
  misc_feature 1..1053: note = Description of Artificial Sequence: Synthetic polynucleotide
  source 1..1053: mol_type = other DNA
ctgagcccccgaaccctcaacgcatgggtcaaggtgatcgaagagaaggcgttctctcctgaagttattcctatgtttacggctctgtctgagggcgccacaccccatgatttgaacacgatgctcaacacgataggcggtcatcaagccgcgatgcagatgctcaaggatacgatcaacgaggaagctgcagagtgggaccgagttcacccagtccatgcagggcctgttgcaccagggcaaatgcgggacccacgcggttcagatattgccggttccacgtctaccctgcaagaacaaatagcgtggatgacaaataatccaccgatcccggtgggcgatatatacaagagatggattatcatgggcctgaacaaaatagttaggatgtacagcccggtgtcaatacttgatataaaacaaggaccgaaagaaccctttcgcgactatgtagaccggttctacaggacgcttagggcagaacaggcctcccaagatgtgaagaattggatgacagagacattgcttgttcagaacagcaaccctgactgcaagaccatacttaaagctctgggtccaggagccactctggaagagatgatgtcagcatgtcagggggtaggcgggccatcacataaagcccgggttttggcggaagcaatgtgccagaaagaaaaaatttatctggcgtgggtgcccgcccacaaggggataggtggtaacgagcagatcgacaagctggtctcaacagaacctatagctggagtagagacgttttatgtggatggtgcaagcaatcgggaaaccaaggcggtaaaggccgcttgctggtgggccggagtcaagcaggagttcggcatcccgtataacacccaaagtcaaggtgtcgtcgaatccatgaataacgagttgaaaaagatcataggtcaaataagggatcaagctgaacatctgaagacggctgttcagatggcggtccttatccacaactttaagcgcaaaggaggtataggagagtacagcgcaggtgaacggataatagacattatagca

SEQ ID NO: 156 - DNA, 1059 bp, synthetic construct
  misc_feature 1..1059: note = Description of Artificial Sequence: Synthetic polynucleotide
  source 1..1059: mol_type = other DNA
ctgtctcctagaacattgaacgcttgggttaaggttatagaagagaaagccttcagtcctgaggtgattcccatgtttaccgccctgagcgagggcgctacaccacatgatctgaataccatgctgaatactatcgggggacatcaggctgccatgcagatgcttaaagatacaataaatgaggaggctgcagagtgggacagggtccatcctgtacacgcgggacctgttgcgccgggacagatgagagatccgcgggggagcgatattgcaggaagcacctcaactcttcaggagcagattgcctggatgacgaacaaccctccgattcctgtgggagacatttataagaggtggataattatggggttgaacaagatagtcaggatgtattctcctgttagcatcctggacataaaacagggccctaaagagccttttcgcgattatgttgacaggttttataggacacttcgcgcggagcaagcctcccaggatgttaaaaactggatgaccgagacgctcctggttcaaaacagtaaccccgattgtaagaccatccttaaagcacttggacctggcgctaccctggaggaaatgatgagcgcctgtcagggggtaggaggcccatcacataaggcacgggtgctcgcagaggcgatgtgtcaagcggtgaaggcagcctgctggtgggcaggtgtgaagcaggaatttgggattccttataatacacaatcccaaggtgttgtcgaatctatgaacaatgaactgaagaaaattatagggcaaatccgggaccaagctgagcacctcaaaaccgcggttcaaatggctgtacttattcataatttcaagcgcaaagggggaatcggtgagtacagtgctggggaacggataatagatattatcgccaccgagcctatcgcaggtgttgaaactttttacgtggacggtgcatcaaacagagagaccaaagcggccaaggagaagatatatttggcctgggttcctgctcacaagggaattggcggaaatgagcaaattgataagctcgtaagt

SEQ ID NO: 157 - DNA, 1092 bp, synthetic construct
  misc_feature 1..1092: note = Description of Artificial Sequence: Synthetic polynucleotide
  source 1..1092: mol_type = other DNA
gttgccaaggagattgtggcctgctgtgacaagtgccaattgaagggggaggcaattcatggacaggtggattgcagtccaggagtctggcagctggattgcacacacctggaagggaaggtcatccttgttgcagttcatgttgcctcaggatacattgaagcagagatcatccccacagaaactggtcaggaaacagcttacttcattctcaaactggctggcaggtggccagtcaccacaatcaccaagcttcaaaatttcagggtttactacagggacaacagagatcctctctggaagggacctgcaagacttctttggaagggtgaaggggcagttgtcattcaggacaacagtgagatcaaggttgttcccaggagaaaggtcaagatcatcagggactatgggaaaaggatggcaggggatgactgtgttgcagggagacaagatgaggatggcactgtgctgataggccccacacctgtcaacatcattggcaggaacctcttgactcagctgggctgcactctcaattttcccatttcccccattgacacagtgccagtgaaactgaagcctggaatggatggcccaagggtcaaacagtggcccctgacagaggagaagatcaaggctctcattgagatctgcactgaaatggaaaaggaaggcaagatcagcaggattggccctgagaacccttacaacactcctatttttgcaatcaagaagaaggatgggaccaagtggaggaagttggttgacttcagggaactcaacaagaaaacacaagacttttgggaagtccaacttggcattccccatcccagtgggttgaaaaagaaaaagtcagtgacagtgttggacattggggatgcttatttctcagttccccttgacaaggagttcagaaaatacacagcattcactgtgccttccacaaacaatgaaacccctggggtcaggtaccagtacaatgtccttccaatgggttggaaaggcagtcctgcaatctttcaatgcagcatgacaaaaatccttcaagaagaagaggaagttggctttcctgtgaggcctcaggtccccctt

SEQ ID NO: 158 - DNA, 1092 bp, synthetic construct
  misc_feature 1..1092: note = Description of Artificial Sequence: Synthetic polynucleotide
  source 1..1092: mol_type = other DNA
gttgccaaggagatcgtggcgtgctgcgataagtgccaattgaaaggggaggcgattcatggacaggtcgattgtagtcccggagtctggcagctggattgcactcacctggaaggtaaggtcatccttgtggctgtacatgttgcctccgggtatattgaagccgagataatccctaccgaaactggtcaggaaaccgcttacttcattctcaaacttgctggaaggtggcccgttactacaattaccaagctgcaaaatttcagggtgtactacagggataaccgcgaccctctctggaagggcccagcacgactgctttggaagggtgagggggctgttgttattcaggataacagtgagattaaggtagtccccaggcgaaaggttaaaataatacgggactacggtaaaagaatggcgggggatgactgtgttgcagggagacaagacgaagacggaacggtacttataggcccaactccggtaaacataattggtagaaacctcttgacgcagctgggctgtactcttaattttccaatatcacctattgacactgtcccagtcaagctgaagcctggaatggatggacctcgggttaaacagtggcccctcaccgaggagaagatcaaggcgctgatcgaaatctgcaccgaaatggaaaaggaaggtaagattagccggatcggccccgagaacccttataacacgcctatattcgctatcaagaagaaggacggaacaaagtggaggaagttggttgacttccgggaacttaacaagaaaacacaagacttttgggaagtccaactcggcatcccacacccaagtgggttgaaaaagaaaaagtccgtaactgtattggacataggtgacgcttatttttcagtaccacttgataaggaatttcgaaaatacacagcgttcactgtgccgtccacgaacaacgaaacccccggggtacgctaccaatataacgtactgccaatgggttggaaaggtagtcctgcgatctttcaatgcagtatgactaaaatccttcaagaagaagaggaagtcggatttcctgtgcggccccaggtccccctg

SEQ ID NO: 159 - DNA, 1089 bp, synthetic construct
  misc_feature 1..1089: note = Description of Artificial Sequence: Synthetic polynucleotide
  source 1..1089: mol_type = other DNA
gggacggtgcttattgggcccactccagtcaatattatcggacgaaacctgctgactcagctgggttgcactctcaatttccctattagtcctatagacacggtgcccgtaaaactcaagccaggcatggatggtccgcgcgtgaagcaatggcctttgactgaagaaaaaattaaggcactcattgagatatgtacggagatggagaaagaagggaaaatctctcgaattggaccagaaaacccgtacaatactccgatttttgcgattaagaagaaggatggcacgaagtggcgcaaactcgtcgattttagggagcttaacaagaagacccaagatttttgggaggtccagctgggcattccgcatccgtccggcttgaaaaagaaaaaatcagttactgtgcttgatataggagacgcctattttagtgtgccgttggacaaggaatttcgcaaatatacagcatttaccgtcccttcaacgaacaatgagactccgggcgtacgctaccaatataatgtgttgcctatggggtggaaggggtctcctgcgatcttccaatgctctatgacgattaccaagctgcaaaattttcgcgtatactaccgcgacaatcgagatccattgtggaaggggccggcgaggctcctttggaagggagaaggtgccgtagttatccaagataatagcgagatcaaagttgtgccccgaagaaaggtgaaaattataagggattacgggaagagaatggcgggagacgactgtgttgccggtcgccaagacgaggatgttgcgaaggaaatcgttgcgtgttgtgataaatgtcagctgaaaggcgaagcaattcacggacaggtagattgttctcctggcgtgtggcagcttgactgtacccatttggagggaaaagtgatcctggtagcagtccacgttgccagtggctacatagaagctgagataattcctactgagacgggacaggagacggcttattttattctcaagctggcaggcagatggcccgtgaccaccgcggcgcaggaagaggaggaagttggttttcccgtacgccctcaggttcccttg

SEQ ID NO: 160 - DNA, 1173 bp, synthetic construct
  misc_feature 1..1173: note = Description of Artificial Sequence: Synthetic polynucleotide
  source 1..1173: mol_type = other DNA
acagtcaaagccgcttgctggtgggcgggcataaagcaagaatttgggataccctataaccctcaatctcaaggggtagttgaatctatgaacaaggaactcaaaaagatcataggacaggtccgcgatcaggctgagcatttgaagacggctgtgcaaatggcggtttttatccataacttcaagcggaagggtgggatcggcggagcagcaatcagtccacgaactcttaatgcttgggtgaaggttgttgaggagaaagcgttcagtcctgaggtgatccccatgttctctgcacttagcgaaggagcaacaccccaggaccttaacacgatgttgaacacagtggggggtcatcaagccgccatgcagatgctcaaagaaactataaatgaggaggctgccgagtgggacagactgcatcctgtgcacgccggaccaatagcaccggggcagatgcgagaaccgcgaggttccgacatcgctgggaccacttctactctgcaggagcagattggttggatgactaacaaccccccgataccggtgggtgagatctataagcgctggatcatacttggccttaacaaaatagttcgcatgtactcaccaacaagcattctcgacatccgacaggggcctaaggagccttttcgagactatgtggatagattttataaaactttgcgggcggagcaagcatcccaggaggttaagaactggatgacagagacacttctggtccagaatgccaaccccgactgtaaaacgatacttaaagcacttgggccggctgcaactctggaggaaatgatgacagcgtgtcaaggtgtggggggtcctggccataaggctcgcgtgttggcggaagcaatgtcacaagttgccaaagaaatagttgccagttgcgacaagtgccaactcaaaggtgaagcgatgcatggacaggtggattgctcaccaggcatctggcagcttgactgtacacacctggagggcaagataattttggtcgcggtgcatgtagcaagtggttatatcgaagctgaggtaatacccgccgagacggggcaagagacagcctacttcctcttgaagttggccggtcgatggccggttaagacgcaagaggaggaagaggttggcttccccgttaagcctcaagtaccgctt

SEQ ID NO: 161 - DNA, 1179 bp, synthetic construct
  misc_feature 1..1179: note = Description of Artificial Sequence: Synthetic polynucleotide
  source 1..1179: mol_type = other DNA
gcggtgaaggccgcatgttggtgggcgggtgttaaacaagagttcggtataccgtacaatacgcagagtcaaggagtcgtagaatctatgaataatgagctgaaaaaaatcattgggcaaattcgggaccaggctgagcatctcaaaacggccgttcagatggccgtcctgattcataactttaagagaaaaggcggcataggggagctcagtccaaggactctcaacgcctgggtgaaggttattgaagaaaaagcgtttagcccggaggtaattccaatgtttacagctctcagcgagggggcgacacctcatgatctcaatacaatgctcaatacaataggggggcaccaggccgctatgcaaatgctgaaagacacgatcaatgaagaagcggctgaatgggatagagttcatcctgttcatgcaggaccggtcgccccgggacagatgagagacccgcgcggttccgacatagctgggagcacgtctacgttgcaggagcagatcgcttggatgactaataatccccctatccctgtcggtgatatttataaacggtggattattatgggtttgaacaaaattgtgagaatgtacagcccagtttccatacttgatattaagcaggggccgaaagaaccctttagggactatgtagaccgcttctatcgcacacttagagccgagcaggcgagtcaggacgtaaagaactggatgacagaaaccctccttgtccaaaactccaatcccgattgcaaaaccattttgaaagcactcggtcctggagccactttggaagaaatgatgtccgcgtgtcagggggtgggagggccaagccacaaagcgagagtattggcggaggcgatgtgccaggttaccgtagcgaaggagatagtcgcatgctgtgacaaatgccaacttaaaggcgaggcgatccatggtcaggttgactgcagtccgggggtatggcaacttgactgtacacatttggagggtaaggttattctcgttgcagttcatgtagcttcaggatacatcgaggccgaaatcatcccgacggagacgggccaagagactgcctacttcatcttgaaactggcgggtcgctggccggtaactaccgccgcccaggaggaagaagaagttgggttccctgtccgaccccaagtgccactc

SEQ ID NO: 162 - DNA, 1173 bp, synthetic construct
  misc_feature 1..1173: note = Description of Artificial Sequence: Synthetic polynucleotide
  source 1..1173: mol_type = other DNA
acggtgaaagcggcctgttggtgggcgggaattaagcaggaatttgggataccgtataaccctcaaagccaaggcgtcgtagaatccatgaacaaagagctgaagaagattattggccaggttcgggaccaggcagaacaccttaaaacagccgtgcagatggcagtgttcatccataattttaagcggaagggcgggattggcggagttgcgaaggaaattgttgcgagttgtgataaatgccaacttaaaggggaggcaatgcacggacaagttgattgctcacctggcatatggcagctggattgtacccaccttgagggtaaaataatcctggtggccgttcatgtcgcatctggctatatagaagcggaagtcattccagcagagacgggtcaagaaactgcttactttctccttaaacttgcgggaaggtggcctgttaaaaccgccgctattagccccaggacgttgaatgcctgggtaaaggttgtggaggagaaggcattctcccctgaggtaattcccatgttctcagcactgagtgaaggggctacacctcaagatctgaacacgatgctcaacacggttggcggacatcaagcggccatgcaaatgctcaaagaaaccatcaatgaagaagcggctgagtgggaccgccttcatccagtccatgctggcccaatcgcacctggtcaaatgagagagccgaggggtagtgatatagccgggacgactagcacattgcaggaacagatagggtggatgacaaataaccctcctatacctgtgggggaaatatataaacgctggattattctcggtctgaacaagattgtcaggatgtactccccgaccagtatccttgacataagacaagggcctaaggagccctttcgggactacgttgatcggttctataagacgcttcgggccgagcaggcgtctcaagaggtgaaaaattggatgactgaaactttgctggtgcaaaatgctaaccccgactgcaagacaatattgaaggctctcggtccagcagcaactttggaagagatgatgacagcgtgtcaaggcgtaggtgggccaggacacaaggctagggtccttgcagaggctatgtctcagcaggaggaggaagaggtaggtttccccgtcaagcctcaagtcccactc

SEQ ID NO: 163 - DNA, 1182 bp, synthetic construct
  misc_feature 1..1182: note = Description of Artificial Sequence: Synthetic polynucleotide
  source 1..1182: mol_type = other DNA
cttagtccgagaactctcaacgcttgggtcaaagtcatagaggagaaagccttttcacccgaagtaatacctatgttcactgcgttgagcgagggcgcgacacctcatgacctgaatactatgctgaacaccatcgggggccaccaagcagctatgcagatgctgaaggacacaattaacgaagaagcggcagaatgggatagggttcaccctgtacatgccggaccagttgcacctggccaaatgagagacccacgagggagcgacatcgcaggctcaactagtaccctgcaagagcagatagcgtggatgaccaataatcctcctattcctgttggtgacatttacaaacgatggataataatgggcctcaataagatcgtcagaatgtacagcccagtgagcatcctggatataaagcagggaccgaaagaacccttccgggactatgttgaccgcttctaccggactcttagggcggaacaggccagccaggacgtaaaaaactggatgactgaaacgttgttggttcaaaattcaaatccagactgcaaaaccattctcaaagcactcggaccaggcgctaccctggaagagatgatgtctgcctgtcaaggtgtcggggggccgagtcacaaggcacgcgtactggcggaggccatgtgtcaaccagtagttgccaaagagattgtcgcgtgctgtgataagtgtcagctcaaaggggaagcgatacatggacaagtagactgtagtcctggcgtgtggcagttggactgtacccatttggagggcaaggtaatattggtagctgtccatgtcgcgtctggttatatcgaagcagaaatcattccgactgagactggtcaagagacggcctacttcatactgaaacttgcaggtaggtggccggtaacaacggcggcttatgccgtaaaagctgcttgttggtgggctggagtcaagcaagaatttggaatcccttacaacacacagagtcaaggcgtcgtcgagtctatgaataacgagctgaaaaagatcataggccaaatcagagaccaagccgaacacttgaagacagccgttcaaatggcagttcttatccacaacttcaagcgcaaagggggcataggtgaacaggaggaggaggaggtaggcttccctgtccggccgcaagtgccgctc

SEQ ID NO: 164 - DNA, 3084 bp, synthetic construct
  misc_feature 1..3084: note = Description of Artificial Sequence: Synthetic polynucleotide
  source 1..3084: mol_type = other DNA
acagtgaaggcagcttgttggtgggccggaattaaacaggagtttggcatcccttataatcctcagtctcagggagtggtggagtctatgaacaaggagctgaagaagatcatcggccaggtgagagatcaggcagaacatctgaagacagcagtgcagatggccgtgtttatccacaacttcaagaggaagggcggcattggaggatatagcgcaggagaaagaatcgtggacatcatcgccatctctcctagaacactgaacgcttgggtgaaagtggtggaggagaaagcctttagcccagaagtgatccctatgttctcagctctgtcagaaggagctacacctcaggatctgaacaccatgctgaataccgtgggaggacatcaggcagctatgcagatgctgaaggagacaattaacgaggaagcagccgagtgggatagactgcatccagtgcacgcaggacctattgctccaggacagatgagagagcctagaggaagcgatattgccggcacaacatctacactgcaggaacagatcggttggatgaccaacaatcctcctatcccagtgggcgaaatctacaaacgctggatcatcctgggcctgaataagatcgtgagaatgtacagccccacaagcatcctggatatcagacagggacctaaggaacctttcagggattacgtggaccggttctacaagacactgagagcagaacaggcatctcaggaggtgaagaattggatgaccgagacactgctggtgcagaacgctaatccagattgcaagaccattctgaaagctctgggaccagcagctacactggaagagatgatgacagcttgtcagggagtgggaggaccaggacataaagctagagtgctggcagaagctatgtctcagatggcagctagagcttcagtgctgtcaggaggagaactcgataggtgggagaagatcagactgagaccaggaggcaagaagaagtacagactgaagcacatcgtgtgggcttctagagaactggagagatttgccgtgaatccaggactcctggaaacacctccagtggtggctaaagagattgtggcttcttgcgataagtgccagctgaaaggagaggctatgcacggacaggtggattgttctccaggaatttggcagctggattgtacacacctggagggaaagattattctggtggcagtgcacgtggcatcaggatatattgaggccgaagtgattccagcagaaacaggacaggagacagcttactttctgctcaaactggcaggtcgctggccagtgaaaaccaaggagaaggtgtacctggcttgggtgccagctcataaaggaattggcggaaacgagcaggtggataaactggtgtctacacagggctacttcccagattggcagaattacacaccaggaccaggcacaagatatcctctgacattcggttggtgtttcaagctcgtgccagtgagagctaaaagagctccagtgaagcagaccctgaatttcgatctgctgaagctcgcaggagacgtggaatctaatccaggacctctgtctcctagaacactgaacgcttgggtgaaggtgatcgaagagaaggcctttagcccagaagtgatccctatgtttacagccctgagcgaaggagctacacctcacgatctgaataccatgctgaacacaattggaggacatcaggccgctatgcagatgctgaaggacaccatcaacgaagaagcagccgagtgggatagagtgcatccagtgcacgcaggaccagtggctccaggacagatgagagatcctagaggaagcgacatcgcaggatctacatctacactgcaggaacagatcgcctggatgacaaataacccccctatcccagtgggagatatctataagcgctggatcatcatgggcctgaacaagatcgtgaggatgtacagcccagtgtctatcctggacatcaagcagggacctaaggagccttttagagactacgtggacagattctacagaacactgagagccgaacaggcatctcaggacgtgaagaattggatgaccgagacactgctggtgcagaacagcaaccccgattgcaagacaatcctgaaagccctgggaccaggagctacactggaagagatgatgtcagcttgtcagggagtgggaggaccatctcataaggctagagtgctggcagaagctatgtgtcaggtggctaaggagattgtggcttgttgcgacaagtgccagctgaaaggagaagctatccacggacaggtggattgttctccaggagtctggcagctggattgtacacacctggaaggaaaagtgattctggtggcagtgcacgtggccagcggctatattgaagccgagatcattcctacagagacaggacaggagacagcttacttcattctgaaactggcaggtcgctggccagtgacaacaatggcagctagagcttctatcctgtcaggaggaaagctcgataagtgggaaaagatcagactgagaccaggcggaagaaagaagtacaagctgaagcacctcgtctgggcttctagagaactggaaagattcgccctgaatccaggtctgctggaaacagcagcagcagtgaaagcagcttgttggtgggcaggagtgaaacaggagttcggcatcccttacaacacccagtctcagggagtggtggaatctatgaacaacgagctgaagaagatcatcggccagatcagagaccaggcagaacatctgaagacagcagtgcagatggcagtgctgattcacaacttcaagagaaagggcggcattggagagtatagcgccggagagagaatcatcgatatcattgccacacagggcttcttcccagattggcagaattacaccccaggaccaggcattagatttcctctgaccttcggttggtgtttcaaactggtgcccctgctgatcaagaaggagaagatctacctggcttgggtgccagctcataaaggaatcggaggaaacgagcagatcgataaactggtgtct

SEQ ID NO: 165 - DNA, 3066 bp, synthetic construct
  misc_feature 1..3066: note = Description of Artificial Sequence: Synthetic polynucleotide
  source 1..3066: mol_type = other DNA
tttcctcagatcactctctggcagagaccactggtgacaatcaagatcggaggacagctgaaagaagctctgctggatacaggagcagacgatacagtgctggaagagatgaatctgccaggtcgctggaaacctaagatgattggaggcattggcggctttatcaaggtgagacagtacgaccaggaggaagtgggatttccagtgaaacctcaggtgcctctgagacctatgacatttaagggcgctctggacctgtctcactttctgagagagaagggaggactggaaggactgatccctaagttcaagctgcctatccagaaggagacttgggaaacttggtggacagagtattggcaggctacttggattcccgagtgggaatttgtgaacacacctcctctggtgaagctgtggtatcagctggaaaaggagcctattgtgggcgcagaaacattctacgtggacggagcagctaacagagaaactaagtggggattcaccaccccagataagaagcaccagaaggagccaccatttctctggatgggatacgaactgcacccagataagtggacagtccagcctattgtgctgccagaaaaggactcttggaccgtgaacgatatccagaagctggtgggaaagctgaattgggcttctcagatctacccaggaatcaaggtgatcaccaagatccagaacttcagggtgtactacagagacagcagagatcctctctggaagggaccagctaaactcctctggaaaggagaaggagcagtggtgatccaggataacagcgacatcaaggtggtgcctagaagaaaggccaagatcatcagggactacggcaaacagatggcaggagacgattgcgtggcttctagacaggacgaagacggaacagtcctggtgggacctacaccagtgaatatcatcggcagaaatctcctgacacagatcggttgtaccctgaacttccctatcagccctatcgagacagtgccagtgaaactgaagccaggaatggacggacctaaagtcaagcagtggcctctgacagaagagaagatcaaggccctggtggagatttgcacagagatggagaaggagggaaagatcagcaagatcggcccagagaatccttacaacaccccagtgttcgccatcaagaagaaggatagcaccaagtggagaaagctggtggatttcagggagctgaacaagagaacccaggacttttgggaagtgcagctgggcatcccccatccagcaggactgaagaagaagaagagcgtgacagtgctggacgtgggagacgcttattttagcgtgcctctggacaaggacttcagaaagtacaccgccttcaccatcccttctatcaacaacgagaccccaggcatcagataccagtataacgtgctgcctcagggttggaaaggatctccagcaatcttccagagcagcatgaccagagctaagagagctccagtgaaacagaccctgaacttcgatctgctcaaactggcaggagacgtggaaagcaatccaggacctaatctgcctcagatcacactgtggcagagacctattgtgaccatcaagattggcggccagattaaagaagccctgctggatacaggagcagacgatacagtgctggaggatatgaacctgccaggtaagtggaagcctaagatgattggcggaattggcggctttatcaaggtcaagcagtacgatcagtggggactgacaacaccagacaagaagcaccagaaggaccccccattcctctggatgggatacgaactgcatccagataggtggacagtgcagccaattgaactgccagagaaggagtcttggaccgtgaacgatatccagaagctgatcggcaagctgaattgggcttctcagatctacgcaggaattaaggtggccgctcaggaagaagaggaagtgggatttccagtgagacctcaggtgcctctgagacctatgacatacaagggagctctggacctgtctcactttctgaaggagaagggaggactggaaggaatcaccaagctgcagaatttccgggtgtactacagggacaatagagaccctctctggaaaggaccagctagactgctgtggaaaggagaaggagcagtggtgatccaggataatagcgagatcaaggtggtgcctaggagaaaggtgaagatcatcagggactacggcaagagaatggcaggagacgattgcgtggcaggaagacaggacgaggaccccaagttcagactgcctatccagaaggagacttgggatacttggtggaccgattattggcaggcaacttggattcccgagtgggagtttacaaatacccctcctctggtcaagctctggtatcagctggaaacagagcctattgccggagtggaaaccttttacgtggacggagccagcaacagagagacaaaagccgcaggaacagtgctgattggacctaccccagtgaacatcatcggaagaaacctgctgacacagctgggttgtacactgaacttccctatcagccctattgatacagtgccagtgaagctgaaaccaggaatggacggacctagagtcaagcagtggcctctgacagaagagaagatcaaggccctgatcgagatttgcaccgaaatggagaaggagggcaagattagcaggatcggcccagagaatccttacaatacccctatcttcgccatcaagaagaaggacggcaccaagtggagaaaactggtggatttcagggagctgaacaagaagacccaggacttttgggaggtgcagctgggcatcccccatccttcaggactgaagaagaagaagagcgtgacagtgctggacattggagacgcttactttagcgtgccactggacaaggagttcagaaagtacaccgccttcacagtgcctagcacaaataacgagaccccaggagtgagataccagtataacgtgctgccaatgggctggaaaggaagcccagctatctttcagtgtagcatgaca

SEQ ID NO: 166 - DNA, 3102 bp, synthetic construct
  misc_feature 1..3102: note = Description of Artificial Sequence: Synthetic polynucleotide
  source 1..3102: mol_type = other DNA
gtggccaaagaaattgtggcctcttgcgataagtgccagctgaaaggagaggctatgcacggacaggtggattgttctccaggaatttggcagctggattgtacacacctggagggaaagattattctggtggcagtgcacgtggcatcaggatatattgaggccgaagtgattccagcagaaacaggacaggagacagcttactttctgctcaaactggcaggtcgctggccagtgaagacaacacagggctactttcctgattggcagaattacacaccaggaccaggaacaagataccctctgacctttggttggtgcttcaaactggtgcccgtgacagtgaaagcagcttgttggtgggcaggaattaagcaggagttcggcatcccttacaatcctcagtctcagggagtggtggaatctatgaacaaggagctgaagaagatcatcggccaggtgagagatcaggcagaacatctgaagacagcagtgcagatggcagtgttcatccacaacttcaagcggaagggaggaattggaggatatagcgcaggagagagaatcgtggatatcattgccgccgctatggcagctagagccagcgtgctgagcggaggagaactcgatcgctgggaaaagatcagactgagaccaggaggcaagaagaagtacagactgaagcacatcgtctgggcttctagagaactggagagatttgccgtgaatccaggactgctggaaacaagcgagggcatttctcctagaaccctgaacgcttgggtgaaagtggtggaagaaaaagccttctctccagaggtgatccctatgtttagcgctctgtcagaaggagctacacctcaggatctgaacaccatgctgaacacagtgggaggacatcaggcagctatgcagatgctgaaggagacaattaacgaagaagccgccgagtgggatagactgcatccagtgcacgcaggacctattgctccaggacagatgagagagcctagaggaagcgatattgccggaacaacaagcacactgcaggaacagatcggttggatgaccaataatccccctattccagtgggcgagatctataagcgctggattatcctgggcctgaacaagatcgtgagaatgtacagccccacctctatcctggatatcagacagggccctaaggaacctttcagagactacgtggacaggttctacaagacactgagagcagaacaggcatctcaggaggtgaagaattggatgaccgagacactgctggtgcagaacgccaatccagattgcaagacaattctgaaagccctgggaccagcagctacactggaagagatgatgaccgcttgtcagggagtgggaggaccaggacataaagctagagtgctggcagaagctatgtctcaggcagcagctaaggagaaagtgtatctggcttgggtgccagcccataaaggaattggaggaaacgagcaggtggataagctggtgtctagagctaagagagctccagtgaagcagaccctgaactttgatctgctcaagctggcaggagacgtggaatctaatccaggacctcatcaggctctgtctcctagaacactgaacgcttgggtgaaggtgatcgaagagaaggcctttagcccagaagtgatccctatgtttacagccctgagcgaaggagctacacctcacgatctgaataccatgctgaacacaattggaggacatcaggccgctatgcagatgctgaaggacaccatcaacgaggaagcagcagagtgggatagagtgcatccagtgcatgcaggaccagtggctccaggacagatgagagatcctagaggaagcgatatcgcaggatctacaagcacactgcaggaacagatcgcttggatgaccaataacccacctatcccagtgggagacatctacaagcgctggatcatcatgggactgaacaagatcgtgaggatgtacagcccagtgtctatcctggatatcaagcagggacctaaggagcctttcagagattacgtggacaggttttacagaaccctgagagccgaacaggcatctcaggacgtgaagaattggatgaccgaaacactgctggtgcagaatagcaacccagattgcaagacaatcctgaaagccctgggaccaggagctacactggaagaaatgatgagcgcttgtcagggagtgggaggaccatctcataaggctagagtgctggcagaagctatgtgtcaggcagtgaaagcagcttgttggtgggcaggagtgaaacaggagtttggcatcccttacaacacacagtctcagggagtggtggaatctatgaacaacgagctgaagaagatcatcggccagatcagagaccaggcagaacatctgaagacagcagtgcagatggcagtgctgattcacaacttcaagagaaagggcggcattggagagtatagcgccggcgagagaattatcgacatcatcgccaaggagaagatctatctggcttgggtgccagcccataaaggaattggaggcaatgagcagatcgataaactggtgtcagcagcttatatggccgctagagcctctattctgagcggaggaaagctcgataagtgggagaagatcagactgagaccaggaggcagaaagaagtacaagctgaagcatctcgtctgggcttctagagaactggagagattcgctctgaatccaggactgctggaaacaacacagggcttcttccccgattggcagaattacacaccaggaccaggaatcagattccctctgaccttcggttggtgttttaagctggtgcctctggtggctaaggaaattgtggcttgttgcgacaagtgccagctgaaaggagaagctatccacggacaggtggattgttctccaggagtctggcagctggattgtacacacctggagggaaaagtgattctggtggcagtgcacgtggcatcaggatatattgaggccgagatcattcctacagaaacaggacaggagacagcctactttatcctgaagctggctggtaggtggccagtgacaaca

SEQ ID NO: 167 - DNA, 3060 bp, synthetic construct
  misc_feature 1..3060: note = Description of Artificial Sequence: Synthetic polynucleotide
  source 1..3060: mol_type = other DNA
atcaccaagatccagaacttcagggtgtactacagagacagcagagatcctctctggaagggaccagctaaactcctctggaaaggagaaggagcagtggtgatccaggataacagcgacatcaaggtggtgcctagaagaaaggccaagatcatcagggactacggcaaacagatggcaggagacgattgcgtggcttctagacaggacgaagacgcagcttacgaagaggaagaggtgggatttccagtgaaacctcaggtgcctctgagacctatgacattcaagggagctctggatctgtctcacttcctgagagaaaagggaggactggaaggagcagcttggggatttaccaccccagacaagaagcaccagaaggaaccaccattcctctggatgggatacgaactgcacccagataagtggacagtccagcctattgtgctgccagaaaaggactcttggaccgtgaacgatatccagaagctggtgggaaagctgaattgggcttctcagatctacccaggaatcaaggtgcccaagttcaagctgcctatccagaaggagacttgggaaacttggtggacagagtattggcaggctacttggattcccgagtgggaatttgtgaacacacctcctctggtgaagctgtggtatcagctggaaaaggagcctatcgtgggcgcagaaacattttacgtggacggagccgccaatagagaaaccaagtttcctcagatcactctctggcagagacctctggtgacaatcaagatcggcggacagctgaaagaggctctgctggatacaggagcagacgataccgtgctggaagaaatgaatctgccaggtaggtggaagcctaagatgattggcggaattggcggcttcatcaaggtgagacagtacgatcagggaacagtgctggtgggacctactccagtgaacatcatcggaaggaacctgctgacacagatcggctgtacactgaacttccctatcagccctatcgagacagtgccagtgaaactgaagccaggaatggacggacctaaagtcaaacagtggcctctgacagaggagaaaatcaaagccctggtggagatttgtaccgagatggagaaggagggcaagatttctaagatcggaccagagaacccctacaataccccagtgtttgccatcaagaagaaggacagcaccaagtggaggaagctggtggattttagggagctgaacaagaggacacaggacttttgggaagtgcagctgggcatcccccacccagccggactgaaaaagaagaagtcagtgacagtgctggacgtgggagatgcctattttagcgtgcctctggataaggacttcaggaagtacaccgccttcacaatccctagcatcaacaacgagaccccaggaatcagataccagtacaacgtgctgcctcagggttggaaaggatctccagccatctttcagagcagcatgacaagagccaagagagctccagtgaagcagaccctgaatttcgatctgctgaagctcgcaggagacgtggaatctaatccaggacctcccaagttcagactgcctattcagaaggagacttgggacacttggtggaccgattattggcaggcaacttggattcccgagtgggagttcacaaatacacctcctctggtcaagctctggtatcagctggaaacagagcctatcgcaggagtggaaacattctacgtggacggagcttctaacagagagaccaaggaggaggtgggatttccagtgagacctcaggtgcctctgagacctatgacatacaagggagccctggatctgtctcactttctgaaggagaaaggcggactggaaggactgcctcagattactctctggcagaggcctattgtgacaatcaagatcggcggacagatcaaagaagccctgctggatacaggagcagacgatacagtgctggaggatatgaacctgccaggcaagtggaaacctaagatgatcggaggaatcggcggatttatcaaggtgaagcagtacgaccagatcaccaagctgcagaacttcagggtgtactacagagacaacagagaccctctctggaaaggaccagctagactcctctggaaaggagaaggagcagtggtgatccaggataatagcgagatcaaggtggtgcctaggagaaaggtgaagatcatccgggactacggcaaaagaatggcaggagacgattgcgtggcaggaagacaggacgaagattggggactgacaaccccagataagaagcaccagaaggaccccccattcctctggatgggatacgaactgcatccagataggtggacagtgcagccaattgaactgccagaaaaggagtcttggacagtgaacgacatccagaagctgatcggcaagctgaattgggcttctcagatctacgccggaattaaggtgaagggaacagtgctgattggacctacaccagtgaacatcatcgggagaaacctgctgacacagctgggttgtacactgaacttccctatcagccctatcgatacagtgccagtgaaactgaagccaggaatggacggacctagagtgaaacagtggcctctgacagaagagaagatcaaggccctgatcgagatttgtacagagatggagaaggagggcaagatctctagaattggcccagagaacccctacaatacccctatctttgccatcaagaagaaggacggcaccaagtggagaaagctggtggatttcagggagctgaacaagaagacccaggacttttgggaagtgcagctgggcatcccccaccctagcggactgaaaaagaagaagagcgtgaccgtgctggatattggagacgcctattttagcgtgccactggataaggagttcagaaagtacaccgcctttaccgtgccttctaccaataacgagacaccaggagtgagataccagtacaacgtgctgcctatgggttggaagggatcaccagccatctttcagtgtagcatgaca

SEQ ID NO: 168 - protein, 27 aa, synthetic construct
  REGION 1..27: note = Description of Artificial Sequence: Synthetic peptide
QNLQGQMVHQAISPRTLNAWVKVVKAF

SEQ ID NO: 169 - protein, 27 aa, synthetic construct
  REGION 1..27: note = Description of Artificial Sequence: Synthetic peptide
QNLQGQMVHQAISPRTLNAWVKVEKAF

SEQ ID NO: 170 - protein, 27 aa, synthetic construct
  REGION 1..27: note = Description of Artificial Sequence: Synthetic peptide
QNLQGQMVHQAISPRTLNAWVKIVKAF

SEQ ID NO: 171 - protein, 27 aa, synthetic construct
  REGION 1..27: note = Description of Artificial Sequence: Synthetic peptide
QNIQGQMVHQAISPRTLNAWVKIEKAF

SEQ ID NO: 172 - protein, 27 aa, synthetic construct
  REGION 1..27: note = Description of Artificial Sequence: Synthetic peptide
QNIQGQMVHQAISPRTLNAWVKVVKAF

SEQ ID NO: 173 - protein, 27 aa, synthetic construct
  REGION 1..27: note = Description of Artificial Sequence: Synthetic peptide
PNIQGQMVHQAISPRTLNAWVKIVKAF

SEQ ID NO: 174 - protein, 9 aa, synthetic construct
  REGION 1..9: note = Description of Artificial Sequence: Synthetic peptide
QNLQGQMVH

SEQ ID NO: 175 - protein, 9 aa, synthetic construct
  REGION 1..9: note = Description of Artificial Sequence: Synthetic peptide
QNIQGQMVH

SEQ ID NO: 176 - protein, 9 aa, synthetic construct
  REGION 1..9: note = Description of Artificial Sequence: Synthetic peptide
PNIQGQMVH

SEQ ID NO: 177 - protein, 10 aa, synthetic construct
  REGION 1..10: note = Description of Artificial Sequence: Synthetic peptide
AIIIIIIIIK

SEQ ID NO: 178 - protein, 10 aa, synthetic construct
  REGION 1..10: note = Description of Artificial Sequence: Synthetic peptide
GIIIIIIIIH

SEQ ID NO: 179 - protein, 10 aa, synthetic construct
  REGION 1..10: note = Description of Artificial Sequence: Synthetic peptide
GIIIIIIIIR

SEQ ID NO: 180 - protein, 9 aa, synthetic construct
  REGION 1..9: note = Description of Artificial Sequence: Synthetic peptide
GIIIIIIII

SEQ ID NO: 181 - protein, 9 aa, synthetic construct
  REGION 1..9: note = Description of Artificial Sequence: Synthetic peptide
AIIIIIIII

SEQ ID NO: 182 - protein, 9 aa, synthetic construct
  REGION 1..9: note = Description of Artificial Sequence: Synthetic peptide
IIIIIIIIK

SEQ ID NO: 183 - protein, 9 aa, synthetic construct
  REGION 1..9: note = Description of Artificial Sequence: Synthetic peptide
IIIIIIIIH

SEQ ID NO: 184 - protein, 9 aa, synthetic construct
  REGION 1..9: note = Description of Artificial Sequence: Synthetic peptide
IIIIIIIIR

SEQ ID NO: 185 - protein, 9 aa, Human immunodeficiency virus 1
HPPQAGPVA

SEQ ID NO: 186 - protein, 9 aa, Homo sapiens
VPLQAGPVQ

SEQ ID NO: 187 - protein, 9 aa, Homo sapiens
MPGQAGPVG

SEQ ID NO: 188 - protein, 9 aa, Homo sapiens
QYRQAGPVI

SEQ ID NO: 189 - protein, 69 aa, Human immunodeficiency virus 1
TVKAACWWAGIKQEFGIPYNTQSQGVVESMNNELKKIIGQIRDQAEHLKTAVQMAVLIHNFKRKGGIGE

SEQ ID NO: 190 - protein, 9 aa, synthetic construct
  REGION 1..9: note = Description of Artificial Sequence: Synthetic peptide
AIIIIIIIS

SEQ ID NO: 191 - protein, 9 aa, synthetic construct
  REGION 1..9: note = Description of Artificial Sequence: Synthetic peptide
MIIIIIIII

SEQ ID NO: 192 - protein, 9 aa, synthetic construct
  REGION 1..9: note = Description of Artificial Sequence: Synthetic peptide
IIIIIIISK

SEQ ID NO: 193 - protein, 10 aa, synthetic construct
  REGION 1..10: note = Description of Artificial Sequence: Synthetic peptide
AIIIIIIISK

SEQ ID NO: 194 - protein, 10 aa, synthetic construct
  REGION 1..10: note = Description of Artificial Sequence: Synthetic peptide
MIIIIIIIIR

SEQ ID NO: 195 - protein, 10 aa, synthetic construct
  REGION 1..10: note = Description of Artificial Sequence: Synthetic peptide
GIIIIIIIIK

SEQ ID NO: 196 - protein, 10 aa, synthetic construct
  REGION 1..10: note = Description of Artificial Sequence: Synthetic peptide
AIIIIIIIIH

SEQ ID NO: 197 - protein, 4 aa, synthetic construct
  REGION 1..4: note = Description of Artificial Sequence: Synthetic peptide
YMDD

SEQ ID NO: 198 - protein, 6 aa, synthetic construct
  REGION 1..6: note = Description of Artificial Sequence: Synthetic peptide
KILQEE

SEQ ID NO: 199 - protein, 5 aa, synthetic construct
  REGION 1..5: note = Description of Artificial Sequence: Synthetic peptide
AAQEE

SEQ ID NO: 200 - protein, 734 aa, synthetic construct
  REGION 1..734: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQKEPIVGAETFYVDGAANRETKAAKEKVYLAWVPAHKGIGGNEQVDKLVSRAKRAPVKQTLNFDLLKLAGDVESNPGPLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIATEPIAGVETFYVDGASNRETKAAKEKIYLAWVPAHKGIGGNEQIDKLVS

SEQ ID NO: 201 - protein, 757 aa, synthetic construct
  REGION 1..757: note = Description of Artificial Sequence: Synthetic polypeptide
ITKIQNFRVYYRDSRDPLWKGPAKLLWKGEGAVVIQDNSDIKVVPRRKAKIIRDYGKQMAGDDCVASRQDEDAAAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTVAKEIVASCDKCQLKGEAMHGQVDCSPGIWQLDCTHLEGKIILVAVHVASGYIEAEVIPAETGQETAYFLLKLAGRWPVKTQEEEEVGFPVKPQVPLRAKRAPVKQTLNFDLLKLAGDVESNPGPVAKEIVACCDKCQLKGEAIHGQVDCSPGVWQLDCTHLEGKVILVAVHVASGYIEAEIIPTETGQETAYFILKLAGRWPVTTITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKILQEEEEVGFPVRPQVPL

SEQ ID NO: 202 - protein, 746 aa, synthetic construct
  REGION 1..746: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQKEPIVGAETFYVDGAANRETKAAKEKVYLAWVPAHKGIGGNEQVDKLVSRAKRAPVKQTLNFDLLKLAGDVESNPGPITKIQNFRVYYRDSRDPLWKGPAKLLWKGEGAVVIQDNSDIKVVPRRKAKIIRDYGKQMAGDDCVASRQDEDAAAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTVAKEIVASCDKCQLKGEAMHGQVDCSPGIWQLDCTHLEGKIILVAVHVASGYIEAEVIPAETGQETAYFLLKLAGRWPVKTQEEEEVGFPVKPQVPL

SEQ ID NO: 203 - protein, 745 aa, synthetic construct
  REGION 1..745: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAVKAACWWAGVKQEFGIPYNTQSQGVVESMNNELKKIIGQIRDQAEHLKTAVQMAVLIHNFKRKGGIGEYSAGERIIDIIATEPIAGVETFYVDGASNRETKAAKEKIYLAWVPAHKGIGGNEQIDKLVSRAKRAPVKQTLNFDLLKLAGDVESNPGPVAKEIVACCDKCQLKGEAIHGQVDCSPGVWQLDCTHLEGKVILVAVHVASGYIEAEIIPTETGQETAYFILKLAGRWPVTTITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKILQEEEEVGFPVRPQVPL

SEQ ID NO: 204 - protein, 716 aa, synthetic construct
  REGION 1..716: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQKEPIVGAETFYVDGAANRETKVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSQEEEEVGFPVKPQVPLITKIQNFRVYYRDSRDPLWKGPAKLLWKGEGAVVIQDNSDIKVVPRRKAKIIRDYGKQMAGDDCVASRQDEDAAAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 205 - protein, 715 aa, synthetic construct
  REGION 1..715: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTKEKIYLAWVPAHKGIGGNEQIDKLVSTEPIAGVETFYVDGASNRETKITKLQNFRVYYRDNRDPLWKGPARLLWKGEGAVVIQDNSEIKVVPRRKVKIIRDYGKRMAGDDCVAGRQDEDAVKAACWWAGVKQEFGIPYNTQSQGVVESMNNELKKIIGQIRDQAEHLKTAVQMAVLIHNFKRKGGIGEYSAGERIIDIIAQEEEEVGFPVRPQVPLGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKIL

SEQ ID NO: 206 - protein, 1030 aa, synthetic construct
  REGION 1..1030: note = Description of Artificial Sequence: Synthetic polypeptide
MTVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPVRAKRAPVKQTLNFDLLKLAGDVESNPGPMLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVS

SEQ ID NO: 207 - protein, 1023 aa, synthetic construct
  REGION 1..1023: note = Description of Artificial Sequence: Synthetic polypeptide
MFPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTRAKRAPVKQTLNFDLLKLAGDVESNPGPMLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 208 - protein, 1023 aa, synthetic construct
  REGION 1..1023: note = Description of Artificial Sequence: Synthetic polypeptide
MTVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPVRAKRAPVKQTLNFDLLKLAGDVESNPGPMFPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 209 - protein, 1030 aa, synthetic construct
  REGION 1..1030: note = Description of Artificial Sequence: Synthetic polypeptide
MLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVSRAKRAPVKQTLNFDLLKLAGDVESNPGPMLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 210 - DNA, 2202 bp, synthetic construct
  misc_feature 1..2202: note = Description of Artificial Sequence: Synthetic polynucleotide
  source 1..2202: mol_type = other DNA
acggtgaaggctgcgtgttggtgggccggcataaaacaagaatttggaataccatacaatccacaatctcaaggcgttgtggaatccatgaataaagaattgaaaaaaattatcgggcaagttcgagaccaggctgagcatctcaaaacggccgtacagatggcggtgtttattcacaacttcaaaagaaaaggaggaatcggtggttacagtgcaggcgaacgaatagttgacattatagcgatatctcctcggactctgaatgcgtgggtaaaggtagtcgaggagaaagcatttagccccgaagtcatccccatgttttcagccctttcagagggcgctacaccacaggatctgaataccatgctgaacacagtaggggggcaccaagcggcgatgcagatgctgaaggagacaataaatgaggaggcggcggaatgggatagattgcatcccgtccacgcggggccgatagcgcctggccagatgagggagccacgaggttccgacatcgcgggaacaacctcaaccctgcaggaacaaatagggtggatgacgaataaccctcctattccagttggtgaaatttataaacgatggataatactcggtctcaataaaatagtaaggatgtattctccgacaagcatacttgacatcagacaggggccgaaagaacctttccgcgattatgtggacagattctacaaaacgctcagggccgaacaggccagccaagaggttaaaaactggatgactgaaaccctgctggtccagaatgctaaccccgactgcaagacaatcttgaaggcacttgggccggccgcaacgttggaagaaatgatgacggcttgtcaaggggttggcggtccgggccataaggcccgagtcctggcagaagctatgagtcagaaagaaccaatagttggagccgaaacattctacgtcgacggtgctgcaaatcgcgagacgaaggctgctaaggaaaaggtctatctggcgtgggttccggcacacaagggcataggcgggaatgagcaagtcgacaaattggtctcacgagctaaacgcgccccggttaaacaaaccctgaacttcgacttgcttaaactcgcaggggacgttgaatcaaatccgggcccacttagtccccgaacgctgaacgcatgggtaaaagtaatagaagagaaagcgttttccccggaggttattcccatgtttacagccctcagcgagggcgcgacgccacatgatctcaacacaatgcttaacacgattggggggcatcaggctgcgatgcaaatgctcaaggatacgataaacgaagaggccgcagaatgggaccgagtacatccggtccacgccgggcccgtcgcaccaggacagatgcgagacccccgagggtcagacatcgccggttctacgtcaaccttgcaagaacaaattgcatggatgactaacaatccacctatccccgtgggagatatctataagagatggatcatcatggggcttaataaaatagtcaggatgtattcaccagtttcaattcttgatattaaacagggtcctaaggagccctttcgagattatgtggataggttttatagaacccttcgcgcagaacaggcttcacaagacgtcaaaaattggatgacagaaactcttctcgtacagaattcaaacccggattgtaagacgatcttgaaagcactcggtccgggtgccacgttggaagagatgatgtcagcctgccaaggggtgggtggcccaagccataaggccagggtattggcagaggcaatgtgtcaagctgtcaaggcagcatgctggtgggctggcgtaaagcaggaattcggcataccgtacaatacacagtcacaaggtgttgtcgagagtatgaataatgaacttaaaaaaatcataggacagatccgagatcaagctgaacatctgaagactgctgtacaaatggcagtgcttatacataacttcaaacgcaagggaggaattggggagtattcagcgggagagcggattattgatatcatagcaaccgaacccatagcgggcgtcgagactttctatgtggacggagccagcaatagggagactaaggcggcgaaagaaaagatttatctggcatgggttccggctcataaggggatcggaggcaatgagcagattgacaaactggtatcc

SEQ ID NO: 211 - DNA, 2235 bp, synthetic construct
  misc_feature 1..2235: note = Description of Artificial Sequence: Synthetic polynucleotide
  source 1..2235: mol_type = other DNA
ttgagcccccgaacccttaacgcctgggtgaaggtaattgaggaaaaagctttttcacccgaagtgatcccgatgtttacagcactgtctgaaggtgcaaccccacacgatctcaacactatgctcaataccatagggggccaccaagcagcgatgcagatgctcaaagataccattaacgaggaagcggccgagtgggacagggtacaccccgtgcatgccggacctgtcgccccgggtcaaatgcgagatccacgcggaagtgatatcgctgggagtacgtccaccctccaagaacaaatcgcgtggatgacaaataatcctcctatccccgtaggagatatttataaaaggtggattatcatggggcttaataaaatcgttcgaatgtatagtcctgtctcaatactggacatcaaacaaggcccaaaagaaccattcagggactacgttgacagattctatcgcacactccgagcggaacaagcaagccaggacgtcaaaaattggatgaccgagacgttgcttgtacaaaatagcaaccccgactgtaaaacgatactcaaagccttggggccaggcgcgaccttggaggagatgatgtccgcttgtcagggagtaggcggaccatcacacaaagcacgcgttttggcggaagctatgtgtcaggcggtaaaggcagcctgttggtgggctggagtcaaacaagaatttggcatcccctataatacacagtcccagggtgtcgtcgagtctatgaataatgagctgaagaaaatcattgggcagataagagaccaagccgaacatcttaaaacggctgtccaaatggcggttttgatccataattttaagcgaaaaggggggattggtgagtactcagcaggagagagaatcatagacatcatagcaacagaaccaattgctggtgtcgagactttttacgtagatggggcgagcaatagggaaactaaagcagcgaaggaaaagatttacctcgcgtgggtaccggcccacaagggaatcggcgggaacgagcaaatcgataaacttgtatccagagccaaacgggctccagtaaaacagacactcaatttcgatcttttgaagcttgctggagacgttgagagcaatcctgggccggtagcaaaggagattgtagcttgttgcgacaagtgccagttgaagggtgaagcgatacacggtcaggtcgattgctctccgggagtttggcaacttgactgtacccatctcgagggcaaagttatcctcgtagctgtgcatgtagcatcaggatatatagaggccgagatcattccgacggaaacgggtcaagaaactgcttacttcattctcaaacttgccgggcggtggccagtcacaactatcacgaaactccaaaactttcgagtttactatagggacaatcgagacccactgtggaaaggacctgccaggcttctgtggaaaggggagggtgccgttgtcatacaggataactccgagataaaagttgtgccaaggcgaaaagttaagattattcgggattacgggaaacgcatggcaggggatgactgcgttgcggggcgacaagatgaggatggtactgtacttattggcccaacacccgtgaacattataggacggaatctgctgacacagttggggtgtacgctcaactttccgataagtccgatagatacggttccggtaaagctgaagcccggcatggatggtccgcgcgtgaagcaatggccactcacagaagagaaaatcaaagctttgatagaaatctgcaccgaaatggaaaaagaggggaagatcagcaggatcggcccggagaatccttacaacacccctattttcgcgattaagaaaaaagatggtacaaaatggaggaaactcgttgattttcgggagctcaacaagaaaacgcaagacttctgggaggtccagcttggcataccccacccctctggacttaaaaagaaaaaaagcgtaaccgtacttgatattggtgacgcgtatttctccgttcccttggataaagaatttaggaagtacacggcctttactgtcccctccactaacaacgaaactccgggcgtgcgatatcaatataatgtgcttccgatgggatggaaaggctcaccagcgatttttcaatgcagtatgaccaagattcttcaggaagaagaggaagtggggtttccggtaagaccacaggtgcccctc

SEQ ID NO: 212 - DNA, 2238 bp, synthetic construct
  misc_feature 1..2238: note = Description of Artificial Sequence: Synthetic polynucleotide
  source 1..2238: mol_type = other DNA
acggttaaggcggcgtgttggtgggcggggataaagcaagagttcggaatcccatataatccacaatcccaaggtgtggtggaaagcatgaacaaggaattgaagaagattataggacaagtcagagatcaggccgagcatctgaaaactgcagttcagatggctgtgttcatccacaattttaaacgcaaaggaggaattggtggatatagcgctggcgagaggattgtagacattattgccatatcacctcgcactctgaacgcctgggtgaaggttgttgaagaaaaggctttttcaccggaggtaatcccaatgttcagtgctcttagcgagggggcaactccgcaggaccttaatacaatgttgaacactgtagggggacatcaagccgctatgcaaatgctgaaggagacgattaacgaggaagctgcggaatgggatagacttcaccccgtccacgctggacctattgcaccgggacagatgcgcgaaccaagaggttccgatatagcgggaacaactagcacactccaggaacagataggatggatgaccaacaaccctccgataccagtaggcgaaatctacaagcgctggataatattggggctgaacaaaatcgtcaggatgtacagcccaacttcaatattggacattcgccaaggacctaaagagccgttccgggattacgtggataggttttacaagactttgcgagctgaacaagccagtcaagaggtgaaaaactggatgaccgagactctgctcgtccaaaatgctaatccagattgcaaaacaatacttaaggcactgggtcccgccgcaacgctcgaggagatgatgactgcctgccaaggtgtcggtggtccgggtcacaaagcacgagtcctggcggaagccatgtctcagaaagagcctatagtgggtgccgagacgttctacgttgatggagccgctaatcgagagacgaaagcggccaaggaaaaggtgtatctcgcttgggtgcctgctcataagggcatcggaggtaatgaacaagttgataaactggtgagtcgggcgaagcgcgcaccagtaaagcagacccttaatttcgatttgctcaaactcgctggtgatgtcgaatctaaccccggtccgattacaaaaatccagaattttagggtttactatcgagattcccgagatccactctggaaaggccccgcgaaattgctctggaagggcgaaggggctgtagtaattcaagacaattctgatatcaaggtagtccctcggaggaaagctaaaataatacgagactatggaaaacagatggcgggggatgactgtgtagcaagccggcaagatgaagacgcggcagctataggaacagtgctggtggggccgacccccgtaaacattatcggcaggaatctgttgacgcaaataggttgtacgctcaattttcctatctcaccgatcgaaacggtgcccgtcaagttgaagccgggcatggacggcccaaaggtaaaacaatggcccttgacggaggagaaaatcaaagctcttgtcgaaatctgtaccgaaatggaaaaggaaggtaagataagtaaaatcggaccagaaaacccgtataacactccagttttcgcgataaagaagaaagactccacaaagtggagaaaacttgtagatttcagggagctgaataaaaggacccaggatttttgggaagtccagttgggcataccacatcccgcggggctcaaaaagaagaagtcagtcacggtactcgacgttggcgacgcatatttctctgttccgctcgataaggacttcagaaaatataccgctttcactattccaagtatcaacaatgaaactcccgggatacgctatcaatacaacgttctgccacagggatggaaggggagtccggctatttttcagtcttcaatgacagtggcaaaagagatcgttgcaagctgtgataaatgccaactgaaaggtgaggccatgcacggacaggttgactgctctcccgggatatggcagctggattgcacgcatttggagggtaagataattctcgtcgcggtccacgtggctagtggctacatcgaggccgaagtaatccccgcagagacgggccaagaaactgcgtacttcctcctgaagctggcaggacgatggcctgtcaaaacacaggaggaagaagaagtgggattcccggttaagccccaggttccgctg

SEQ ID NO: 213 - DNA, 2235 bp, synthetic construct
  misc_feature 1..2235: note = Description of Artificial Sequence: Synthetic polynucleotide
  source 1..2235: mol_type = other DNA
cttagcccacggacacttaatgcatgggtcaaggtgattgaggagaaggcgttttccccagaggtgatcccaatgttcacagccctctctgagggcgcaacacctcacgacctgaatacaatgctcaacactatcggagggcaccaagcagccatgcaaatgctgaaagacaccatcaacgaggaggctgctgagtgggatcgagtacaccctgtacacgcggggccggttgctcctggtcaaatgagggatccccgcggctccgacatagccggctcaacaagcactctgcaggaacagatagcatggatgaccaacaacccccctattcccgtaggggacatttacaagaggtggattataatgggtctgaacaagatcgtacgaatgtattcaccggtcagcatacttgatataaaacaaggcccgaaagaacctttcagggactatgtcgatcgattttaccgcacgctgcgcgccgagcaggcctcccaggacgtgaagaactggatgactgaaacactgcttgtgcaaaattcaaatccagattgtaaaaccatacttaaggctcttggtccgggggctaccctggaagagatgatgagcgcgtgtcaaggggtaggaggtccttctcacaaggccagggttttggctgaagctatgtgccaggccgttaaggcggcctgttggtgggcaggagtcaagcaagaattcggcatcccctacaacacgcaatcacagggtgtcgttgaatcaatgaacaacgagctgaaaaaaattataggtcaaatacgcgatcaagcggaacatcttaagaccgcagtgcagatggctgtgcttatacataatttcaagcgaaaaggaggcatcggagaatactcagccggagaaagaatcatcgacattatcgcaacggagccaatcgccggtgtggaaacgttctatgtagacggagcatcaaatagggaaacgaaggccgccaaggagaaaatttatctggcgtgggtcccggcccacaagggaattggcggcaatgaacagatcgacaagcttgtttctcgggctaaacgggctcccgttaagcagacgctcaacttcgatcttctcaagctggccggtgatgtcgagagcaatccggggccggtagctaaggaaatcgtcgcttgttgtgataaatgtcaacttaagggtgaagcaattcatggccaagttgactgttcaccgggagtatggcaactcgattgtacccatctcgaaggaaaagtcatcttggttgcagtgcacgtagcttctggctacattgaagcggagattatcccgacagagaccgggcaggagaccgcttattttatcctcaagctcgcaggacgatggcccgtcactactatcacgaagctccaaaattttagagtgtactaccgcgataacagagatcctttgtggaaaggccccgccagacttttgtggaagggtgagggagcggtggttattcaggacaattctgagataaaagttgtaccccgacggaaggtcaagataattagagattatggtaaaaggatggcgggcgacgactgtgtagctggaaggcaggatgaggacggcacagtgctgattggcccgacgcccgtaaacattatcggtcgcaaccttctcacccagctggggtgcactttgaatttcccgatttccccgattgatactgttccagtaaagctcaaacccgggatggacggcccacgagtaaaacaatggccattgacagaggagaagattaaggcgcttatcgaaatatgtactgaaatggagaaggaagggaaaattagtcggatagggcctgagaatccctacaacacgcccatttttgctatcaagaagaaagatggcaccaagtggcggaagctggtcgattttcgggaacttaacaagaaaacacaagacttctgggaagtacagcttgggatcccgcacccgtcaggattgaagaagaaaaagagcgtcacggtactcgacataggcgacgcttacttctcagttccgctggacaaagagttcagaaaatatacagctttcacggtaccctccactaacaatgagacacctggagttcgctaccagtacaatgtgcttccaatgggatggaagggctcaccggctattttccaatgctctatgactaaaatacttcaagaggaagaagaggttgggtttcccgtcagaccgcaggttccactt

SEQ ID NO: 214 - DNA, 2148 bp, synthetic construct
  misc_feature 1..2148: note = Description of Artificial Sequence: Synthetic polynucleotide
  source 1..2148: mol_type = other DNA
acggtgaaagcagcttgctggtgggcggggatcaaacaggagtttggaataccttataatcctcaatcacagggggttgtcgaaagcatgaacaaggagctcaagaagatcatcggacaggtgcgcgatcaggctgaacatcttaagaccgcagttcagatggcagtctttattcacaattttaaacggaaaggaggtataggtggctacagcgcgggcgagcgcattgtagatattatagcgatttctccgcggacgctgaatgcatgggttaaagtagttgaagagaaggccttttctcccgaagtaatacccatgttcagtgcactgtctgaaggtgctactcctcaggatctcaacacgatgctcaacacggtcggtgggcatcaggcagcaatgcagatgctgaaggaaacgataaacgaggaggcagcagaatgggatcgactgcacccagtacacgcaggccctatagccccaggtcaaatgcgggaaccaagaggtagtgatatagctgggactacctcaacgttgcaggagcaaattggttggatgacgaataatcctcctataccagttggcgaaatatacaaaagatggattatcttgggactgaataaaatcgtgcgaatgtattctccgacctctatactggacattcgacagggaccaaaagagccgttccgcgactacgtcgatcggttttataaaactttgcgggccgaacaggcaagccaggaggtaaagaactggatgacagagaccctgttggtgcaaaatgcgaaccctgattgcaagaccatactgaaagcactcgggccagctgccacccttgaggaaatgatgacagcttgccagggtgtgggggggccggggcataaagcacgcgtcctcgccgaagccatgtcacagaaagaaccaattgtgggtgccgaaactttttacgtggacggcgcagccaaccgagagactaaagtggctaaagagatagttgcatcatgtgataagtgccaattgaaaggtgaggccatgcacggtcaggtagattgttcacctggtatatggcagttggactgtactcaccttgaaggaaagattatcctggtcgcggtacacgtcgcatccggttatatagaggcggaagttatacctgcggagactggtcaagaaactgcctacttccttcttaaattggctggtcgatggccagtaaaaactaaagagaaagtgtaccttgcgtgggttccagcccacaagggtataggaggaaatgagcaagtagacaaactcgtaagccaagaggaagaagaagtgggtttcccagttaagccacaggtacccctcattaccaaaatacagaatttccgggtttattatcgcgattcaagggaccccctgtggaaaggtccagcaaaactgctgtggaagggcgaaggggcagttgttatacaagacaactcagatatcaaggtcgtgccaagacgcaaagctaaaattataagggattatggtaaacagatggctggagacgactgcgtggccagcagacaagacgaggatgcagctgcaattggaacagtcctggtcggaccaactcccgttaacatcataggtagaaacttgctcactcaaatcggatgcacacttaattttccgatttcacctatcgagaccgttcccgttaagctgaaacctgggatggacggtcccaaggtgaagcaatggcccttgactgaggaaaagataaaggcgttggtagagatctgcaccgaaatggagaaggaaggcaagatatctaagatcgggccagaaaacccatataatacacctgtcttcgcgataaaaaaaaaggactctactaaatggagaaaactggtcgacttcagagagcttaataagcgaactcaagacttttgggaagtgcagcttggtatacctcaccctgctggtctgaagaagaaaaaatctgttactgttcttgatgtcggtgacgcatacttcagtgtgcccctcgataaagatttcaggaaatacaccgcgttcactatacccagcattaataacgagacccccgggatacgctaccaatacaatgtcctcccccagggctggaaagggtctccagcaatttttcagtcatcaatgacg

SEQ ID NO: 215 - DNA, 2145 bp, synthetic construct
  misc_feature 1..2145: note = Description of Artificial Sequence: Synthetic polynucleotide
  source 1..2145: mol_type = other DNA
ttgtcccctcggacgctcaatgcatgggttaaagttatcgaggagaaggccttcagtcccgaggttatacctatgttcaccgctctgtctgaaggagcaacgccccatgatctcaatactatgctcaatacaattggaggtcaccaagcggctatgcaaatgctcaaagataccattaatgaggaggctgctgaatgggatagggtccacccagttcatgccggaccggtggctccgggacagatgcgcgacccccgggggtcagacatcgccggaagtacctctactctgcaggaacaaattgcatggatgacaaataatccacctattccggtcggagacatctacaaacgatggatcataatgggtctcaacaagatagtccggatgtatagtccggtaagtatactcgacatcaagcaaggccctaaggagccgttccgggattacgtagaccgattctaccggacgctcagagccgaacaggcctcccaagatgttaagaactggatgaccgaaacgttgttggttcaaaattccaatcctgattgcaaaacgatactcaaagctcttggtcctggtgcaacactggaggaaatgatgtcagcctgccaaggggtcggcgggccttcacacaaagcaagggttttggcggaggcaatgtgccaagtagcgaaggaaatagtggcctgttgtgacaaatgtcagctgaaaggagaggcaatacatggacaagttgactgttctcccggtgtgtggcaactcgactgtacccacttggaaggaaaagttatactggtggccgttcacgtcgcgtctggctacatcgaggctgagatcatacctacagagaccgggcaggagaccgcgtacttcatccttaagctcgcgggccgctggccagtcacgactaaagagaaaatttatctggcgtgggtacccgcgcataaaggtattggcggcaatgaacaaatagacaaattggtatcaacagagccgattgcaggagtcgaaacattctatgttgatggtgcgtcaaacagggaaacgaagataacaaagttgcaaaactttcgagtctactatcgcgacaatcgggatcccctctggaagggcccagcaaggttgctgtggaagggcgagggagcagtagtcattcaagacaacagtgagattaaggtagttccgcgacggaaggtcaaaataatacgggattacggcaaaaggatggcaggggatgattgcgtggctgggcgccaggatgaggacgctgtcaaagccgcgtgttggtgggcaggggttaagcaggagttcggaataccatacaacacccagtctcaaggagttgttgaaagcatgaacaatgagcttaaaaaaataatcggacaaataagggatcaggccgaacacttgaagacagcagttcagatggccgtgctgatccataacttcaaacggaagggcggcataggagaatactccgcaggcgagagaataatcgacattatagctcaagaagaagaggaagtcggctttcctgtacgacctcaggtccctcttggtacggtgttgatagggccgacccccgtcaacatcatcggtcggaacctgcttacacaacttggttgcactcttaactttcctatttcccccatagataccgtcccagtcaagttgaagccggggatggatggcccgcgcgtcaagcagtggcccctgactgaagaaaagattaaagctctgattgaaatatgcacagaaatggaaaaagagggtaagatcagcagaatcggtccagaaaatccctataacacgccgatattcgccattaagaagaaggacggaacaaagtggcggaaactcgtcgattttagggagctgaataagaaaacgcaggatttctgggaagttcaacttggcatacctcacccctctggtcttaaaaaaaaaaagtcagtcaccgttctcgacattggggacgcgtatttttccgttccgctcgacaaagagtttcggaagtacacggcgttcacggtaccttctacaaacaatgaaacccccggggtcaggtatcagtataatgtgctgccaatggggtggaagggtagccctgctatttttcaatgctcaatgacaaagatcctt

SEQ ID NO: 216 - DNA, 3090 bp, synthetic construct
  misc_feature 1..3090: note = Description of Artificial Sequence: Synthetic polynucleotide
  source 1..3090: mol_type = other DNA
atgacagtaaaagcggcttgctggtgggcgggtattaagcaagagtttggtatcccctacaacccccagtcccaaggagtcgtcgagtctatgaacaaagaactgaagaagatcattgggcaggtgcgcgatcaggctgagcacttgaaaacagcggtccagatggctgtgtttatccataactttaagaggaagggggggatagggggctattcagcaggggagagaattgtagacatcatcgccatatcccctcgaacgctcaatgcgtgggttaaagtagttgaggaaaaggcatttagtcctgaagtcatcccaatgtttagcgcactttccgagggcgctacgccccaggacctgaataccatgcttaacaccgttgggggccaccaggcggccatgcagatgctcaaggaaactattaacgaggaggcggcggagtgggatcggctgcaccctgtccacgcaggaccgatcgccccggggcaaatgagagaacccagaggttctgatattgctggaactactagtactcttcaggagcaaatcgggtggatgactaataacccaccaattcccgtaggtgaaatttacaagagatggatcatactgggcttgaacaaaatagtccgaatgtatagtcccacctcaatcctcgacatccggcaaggaccgaaggagcctttccgcgactatgtggatcgcttttataagactctgcgagcagaacaagcatcacaagaggttaaaaactggatgaccgaaacactcttggtgcagaacgcaaatcccgactgcaaaaccatcctgaaggcattgggccctgcagcaactttggaggagatgatgactgcatgtcaaggcgtaggagggcccggccataaagccagagttttggcagaggctatgtctcaaatggcggcacgagcttcagttctgtcagggggcgaacttgatcggtgggaaaagatacggcttcggcccggaggcaagaaaaagtacaggctgaagcacatagtatgggcgtcccgcgaactggagaggtttgcagtgaaccccggcctgctcgagacgcccccggtggttgctaaagaaatagtcgcctcttgtgataaatgccaactcaagggagaagctatgcatggccaggttgactgctcaccgggtatatggcagctggattgtacacatttggaaggtaaaatcatactcgttgctgtgcatgtagcaagcgggtatattgaggcggaagtaattccggcggaaaccgggcaagaaactgcctatttccttcttaaactcgcggggcggtggccggttaagaccaaggagaaagtctatctcgcatgggttccggcccataaaggcatcggcggtaatgaacaagtagataaactcgttagcactcaaggatattttccggattggcagaattatacacccggacctggtacaagatatcccttgacgttcggatggtgtttcaagctcgtcccagtccgcgctaaaagagcaccagtaaagcagaccttgaacttcgacttgctcaagcttgctggggatgtcgaaagtaaccccggcccgatgttgtcccccaggactttgaatgcatgggtcaaagtgattgaggagaaggccttctcccccgaagttattccgatgtttaccgcgcttagtgaaggggccacacctcatgatctgaatacgatgcttaacactatagggggtcaccaggcagcgatgcaaatgctgaaggataccatcaatgaagaagcagctgaatgggacagggtacatccagtgcatgcaggaccggttgcacccggacaaatgcgcgacccgcgaggttccgacatcgcggggtcaacgtccaccctgcaagaacaaattgcatggatgaccaataatccccctatcccagtgggcgacatatataagcgctggataatcatgggtctcaataaaattgtaaggatgtatagtccggtgtcaatcctggacataaagcaaggtcccaaggaaccgtttcgcgactatgtagacagattttatcgaacgctgagagccgagcaagcgagccaggatgtcaaaaactggatgaccgaaacacttctcgttcagaattcaaacccggattgtaagacaatacttaaggcgctcggtcccggggcgacccttgaagagatgatgtctgcttgtcaaggtgttgggggtccatcccacaaagctcgcgtcctggcggaagcaatgtgccaagtcgccaaagaaatcgtcgcgtgctgtgacaagtgccaactcaagggtgaggcgatccatgggcaagtggactgtagtccaggcgtatggcaattggactgtacgcatctcgaagggaaggtgatcttggtggccgtccatgtggcgagcggatatattgaagccgaaatcatccctaccgaaacgggacaagaaacggcgtatttcattttgaaactggcgggtcggtggccggtcaccaccatggccgcgcgagcgagcatacttagcgggggtaaattggacaagtgggagaagatccggcttcggcccgggggtcggaaaaaatataagctgaagcacctggtctgggcatcacgagaactggagcggttcgcacttaatccagggttgcttgaaaccgcagccgcggtgaaggccgcctgctggtgggcgggagtaaagcaggagttcggaattccttacaacacgcagagccaaggtgtagtggaaagcatgaacaatgagcttaagaaaatcattggtcagatcagagaccaagcggaacacctcaagacagctgtgcaaatggctgtacttattcacaacttcaagagaaaaggcggtataggagaatatagcgcgggggaaagaataatagacatcatcgctactcaagggtttttccccgactggcagaattatacacctggccccggtatacggtttccacttactttcggctggtgtttcaagttggtgcctctcctgataaaaaaggagaaaatatatcttgcgtgggtgcctgcacacaaaggtataggcggtaacgaacaaatcgacaaattggttagc

SEQ ID NO: 217 - DNA, 3069 bp, synthetic construct
  misc_feature 1..3069: note = Description of Artificial Sequence: Synthetic polynucleotide
  source 1..3069: mol_type = other DNA
atgttcccccagattacactgtggcagcgacctctggtcaccatcaagattgggggacagcttaaggaagcgctgcttgacactggtgctgatgacactgtactggaggagatgaatctcccgggtcgctggaagcctaaaatgataggcggtatcggggggttcattaaggtcaggcagtacgaccaagaagaggtcggattcccggtaaagccacaagtgcctcttcgcccgatgacgttcaagggtgcgttggacctcagccactttcttcgagaaaagggcggactggaaggtctgatacctaaatttaagctccctattcagaaggaaacatgggagacatggtggacggagtattggcaggcgacatggatccccgagtgggaatttgtaaacaccccgccactcgtaaaactctggtaccaactggaaaaggaacccatcgtcggtgctgaaaccttctacgtcgatggtgcggctaatagggaaacgaagtggggtttcactaccccagacaaaaaacatcaaaaagaaccaccctttctctggatgggatatgaattgcatcccgataaatggaccgtgcaacccatcgttctgccggaaaaggacagctggaccgttaatgatattcagaagcttgttgggaaactcaactgggcttcccaaatttacccgggaataaaggtgataacgaaaattcagaattttagggtgtactatagggactcacgcgatcctctttggaaaggtccagcaaagttgttgtggaaaggtgagggggctgtcgtcatccaagacaatagtgatattaaggtcgtgcctagaagaaaggcaaagattattagggattacggcaagcagatggctggtgacgactgtgttgcaagtcgccaagacgaagatggcacggtgttggtcgggcccacaccagtaaacattataggccgaaatctgcttactcaaatcggatgtactcttaattttccgatctcccctatagaaacggttcctgtaaaattgaaacctggaatggatggtccgaaagttaaacagtggccgctcaccgaggaaaagattaaagcgcttgtcgagatctgtactgaaatggaaaaagaaggaaagatctccaaaatagggccagaaaatccgtacaatactccagtctttgctataaagaagaaggattctacgaagtggaggaagctggtagactttcgcgagctcaacaaacgcacgcaagatttttgggaagtccagttgggcatccctcatccagctggactcaagaaaaaaaaatccgtcacagtattggatgtgggcgacgcctacttttcagtgccattggacaaagattttcgaaaatacaccgcgttcacaattcctagtatcaataacgagactcccggaataaggtaccagtacaacgtgctccctcaagggtggaaaggttctcccgcgatatttcagtccagtatgactcgcgcgaaacgagctccagttaaacagaccctcaactttgatttgttgaagcttgctggggatgttgagagtaatccaggccctatgctgccgcaaatcacactctggcaaaggccgatagtgaccattaaaattggcgggcagatcaaggaggcattgcttgatacgggagcagacgatacagtgttggaggacatgaacctgcccggaaaatggaaaccaaagatgatcggtgggattggcggtttcataaaggtcaagcagtatgaccagtggggtctgacaacccctgacaaaaaacatcagaaggatcccccctttctttggatgggttatgagttgcatccagatcgctggacggtgcagcctattgagcttccggaaaaggagtcttggacagttaatgatattcaaaaacttattgggaaattgaattgggccagccagatatacgcaggtataaaagttgcggcgcaggaggaggaagaagtggggttccccgtccgaccccaggtgccgctcagaccaatgacgtataaaggtgcgttggatctgagtcattttttgaaggaaaaaggcgggttggaaggcattaccaaactccaaaacttccgggtgtattatcgggacaacagagatccactctggaagggtcccgcaagattgctttggaagggagaaggagcagttgttatacaagacaactccgaaattaaggtagtgcctagacggaaggttaaaattattagggactacggaaaacggatggcgggggatgactgcgtcgcgggccgccaggacgaggacccgaaattccgcctgcctatacaaaaggagacgtgggacacgtggtggacagactactggcaagcaacgtggatcccggaatgggaatttactaacacacctcctttggtgaaactctggtatcaactcgagacggagccgattgcaggggtcgagacattttacgtcgatggagcatccaatagggaaactaaagcagctggtacagttctgataggtccgaccccggtgaatataataggcaggaatctcctcacacaacttggctgcactttgaatttcccaatttccccaattgacaccgtacccgtaaagttgaagcctggaatggacggaccacgagtgaagcagtggcctctcacggaagaaaagatcaaagcgcttattgaaatttgtacagaaatggagaaggagggtaaaatctccaggataggtcctgaaaacccgtacaacacgcccatcttcgctatcaaaaaaaaagatggaacgaaatggcgcaagctggtggacttcagagaacttaacaaaaagacgcaggatttttgggaagtccagttgggaatccctcacccgagcggacttaaaaaaaagaaaagtgtcacagttcttgatataggcgacgcttatttttccgtcccacttgacaaggaatttaggaagtacacggcgtttacagtgccatcaacgaacaacgaaaccccgggggtgcgctaccagtacaacgtactgccaatgggatggaaaggttcacccgcaatctttcaatgctcaatgact

SEQ ID NO: 218 - DNA, 3069 bp, synthetic construct
  misc_feature 1..3069: note = Description of Artificial Sequence: Synthetic polynucleotide
  source 1..3069: mol_type = other DNA
atgacggtaaaggcagcatgctggtgggcaggtataaaacaggaattcggcattccgtataacccacaaagtcaaggagttgtcgagtccatgaacaaagaattgaaaaagataattggtcaagtgcgagaccaagcagaacacctgaaaaccgcggttcaaatggccgtgtttatacacaactttaagagaaaagggggcatcgggggctactccgcgggtgaacgcatagtcgatataatagccatctcccctcgcactctcaacgcatgggtgaaagtcgtagaggagaaagctttctcacctgaagtaattccgatgtttagtgcactgagtgaaggcgctacgcctcaagatctgaacacgatgcttaataccgtcgggggtcaccaagccgcgatgcagatgttgaaggaaacaataaatgaggaagcagcagagtgggacagacttcacccggtccacgcgggaccaatcgcaccaggacaaatgcgagaaccgagaggtagtgacatcgccggaacaacctccaccctccaggaacagattggttggatgacaaataatcctccgatacccgtcggtgagatctacaaacgctggatcatcctgggtcttaacaagatcgtacggatgtacagcccaaccagtatccttgacattaggcagggaccgaaggagccgtttcgcgactacgtcgatcggttttacaagacgcttagagcggaacaagcgtcacaggaagttaaaaattggatgacagaaaccttgcttgtccagaatgctaatcccgattgcaaaactattctgaaggcactgggtcctgcggcgactttggaggagatgatgacggcctgtcaaggtgttggaggccctggtcataaggcacgagtcctggctgaagcaatgtctcaaatggcggctagagcctctgtgctgtccggaggggagcttgaccgctgggaaaagatccgattgcgaccaggtgggaaaaagaagtacaggctcaagcatattgtgtgggcatcacgggaacttgagcgcttcgcagtcaatcctggacttcttgaaacgccaccggtggtcgctaaagagatcgttgcgagctgtgataaatgtcaacttaaaggcgaggctatgcatggccaggtcgactgtagcccgggcatctggcagctggattgcactcacctggagggtaagatcattctcgtggcggtccatgttgccagtggctacattgaggcggaggtgattcctgcggaaactggtcaggagacagcctatttcttgctgaagctcgcgggacgctggcctgtcaaaactaaggaaaaggtttatttggcctgggttcccgcacataaaggaattggtggcaatgaacaggtagacaaacttgtaagtactcagggatattttcccgattggcagaattacactccagggccggggactaggtaccctttgacatttggttggtgttttaagcttgtgcctgttcgggcgaagagggcgccagtcaaacagactctgaatttcgacctgctgaagctggcaggagacgtcgagtccaaccctggtcctatgttcccacagattactctgtggcagcgcccgcttgtgactattaaaatcggcggacaactcaaagaggcactccttgacaccggagcggacgacacggtgctggaagaaatgaacttgcccggccggtggaagccaaagatgatcggaggtatcggcggctttataaaggtgcgccagtatgaccaagaagaagtcggcttcccagtaaagcctcaagttccactgagacctatgacttttaagggtgcgcttgatctgtcacacttcctccgagagaaaggcggcttggagggccttattcccaagttcaagttgcctattcaaaaagaaacgtgggagacgtggtggactgaatattggcaggcgacctggatccctgaatgggagttcgtgaacacacccccactcgttaaactctggtatcagttggaaaaggaacccatcgtgggcgccgagacattttacgtcgatggtgccgctaacagagagaccaagtgggggtttacaacgcctgacaagaagcaccagaaggagccccctttcctttggatgggatatgagttgcaccccgacaaatggaccgtgcaaccgattgtcttgcctgagaaagactcttggacagtgaacgatatccaaaaacttgtgggaaaattgaattgggcaagccaaatctacccagggataaaggtaatcactaagattcaaaacttccgagtatactaccgagacagcagagatcccttgtggaaaggtcctgcgaaactgctctggaaaggcgagggagctgtggtcattcaggacaactcagacatcaaagtagtcccacgccgcaaagcgaaaatcatacgcgactatggcaaacaaatggcaggtgatgattgtgtggcgagtcgacaagatgaggatggtaccgttctggtcgggccgacacctgttaatattataggacgcaatttgttgacacaaatcggctgcactcttaacttcccgataagtcccatcgagacagtgccagttaaattgaagccagggatggacggtcctaaggttaagcagtggcccctcactgaagaaaaaatcaaggctctcgtggaaatttgcactgagatggagaaggagggcaaaatctccaagataggtccagagaacccatataatacgccggtatttgcaatcaaaaaaaaggacagcacaaagtggcgaaagctggttgactttcgagagctgaacaagcggacgcaggacttttgggaagtccaattgggaataccgcatcccgctggattgaaaaaaaaaaagagcgtaacagtcctcgatgtaggtgatgcatacttcagtgtcccactcgataaagattttagaaagtacactgcttttacgattccatccattaacaacgagactcccggtattcgatatcaatacaatgtactcccacaaggttggaaaggctcacctgcgatcttccaaagtagcatgact

SEQ ID NO: 219 - DNA, 3090 bp, synthetic construct
  misc_feature 1..3090: note = Description of Artificial Sequence: Synthetic polynucleotide
  source 1..3090: mol_type = other DNA
atgttgtctcctagaactctgaatgcttgggtaaaggtgatcgaagaaaaggcattctcacccgaggttatccctatgtttactgcgttgagcgaaggcgcaacaccccatgatctgaacacaatgctgaatacaatcggcggacatcaagctgctatgcaaatgcttaaggacaccatcaatgaggaggcagccgagtgggatcgcgttcatccagtccacgctgggcccgttgcgcctggtcagatgagggacccacgaggatccgacatcgcagggagcaccagtacactccaagagcagattgcatggatgacgaacaatccaccaatacctgtcggtgacatttacaaaaggtggattattatgggcttgaacaaaattgtacggatgtatagcccggtgagcatactggacattaaacagggtccaaaagaaccctttcgagattacgttgacagattttataggacactgagggcggaacaagcgtctcaagacgttaaaaactggatgacagaaacgctgcttgtacagaattccaacccagactgcaaaactatactgaaagcgctcggtcccggtgcgacacttgaggaaatgatgagcgcatgccagggcgtcggagggccgtctcacaaggcccgcgtgttggcggaagctatgtgccaggtggctaaggagatagtagcatgttgtgataaatgtcagcttaaaggtgaagctatacatgggcaggtggattgtagcccgggtgtatggcagctggactgtactcatctggaaggaaaggtaatacttgtcgcagttcatgtcgcgagcggatacattgaagctgaaatcattcctacggagacgggccaagagacagcttacttcatacttaaacttgctgggcgatggccggtgacaactatggcggcgcgcgcttcaattttgagtggtggaaagttggacaagtgggagaagattagactcagacccggagggagaaagaagtataaactgaaacatctggtttgggcttcacgcgaacttgaacggtttgctctcaaccccgggctgctcgaaactgctgctgctgtaaaggccgcttgttggtgggcgggggtaaagcaggaattcggaattccatataacactcaaagccagggagtagtggaatccatgaataatgaacttaagaagataattggacagattcgcgatcaggctgaacatctcaagacggccgtacaaatggcagtattgattcataactttaaacggaagggcggcataggagagtattctgcgggagaacgcataatagatataattgcgactcaggggttctttccggattggcagaactatacgccggggccaggcattaggttccccctcacgtttggatggtgtttcaagttggtaccgttgctcattaaaaaagaaaaaatctacctggcctgggtcccggcgcacaagggtataggggggaacgagcaaattgataagctcgtgtcaagggcgaagcgcgcgccagtcaaacagaccttgaatttcgacctccttaagctcgctggagacgtcgaatccaaccctggcccgatgctgccacaaatcacattgtggcaacgacccattgtaacaataaaaatcgggggccagatcaaagaagcgctgcttgacaccggcgccgacgatacagtcctcgaggatatgaatttgccaggcaaatggaagccgaagatgattggcggcattggcggctttattaaggttaaacagtatgatcagtggggattgaccactcccgataagaagcatcagaaagatccgccttttctgtggatggggtacgaactgcaccctgatcgatggacggtccagccgatagagctgccggaaaaagaatcatggaccgtgaatgatattcaaaaactgatcggaaaactcaattgggcgtcccagatatatgctggcatcaaagttgcagcacaagaagaggaagaggtaggtttcccggttcggccgcaagttcccttgcgaccgatgacatacaagggcgcattggacctttctcacttcctcaaggaaaagggcggtttggagggcatcactaaacttcagaatttcagagtctactatagagataacagggacccattgtggaagggccccgctcgacttctctggaaaggggagggagcggttgtaattcaagacaacagtgaaattaaggtcgtcccacgacggaaggttaaaataattcgcgactatggcaagcgaatggcgggagacgactgtgtagcaggacgacaagacgaggacccaaagtttagattgccgatccagaaagagacatgggatacgtggtggacggactattggcaggccacctggataccagaatgggagtttacaaacactcctccactcgtgaaattgtggtatcaacttgagaccgagcccatagctggtgtagagacgttttacgttgacggtgctagcaacagggaaacaaaggccgctggaaccgtgctcatcggtcctactcctgtgaacataattggacgaaatttgttgacccagctgggatgcaccctcaatttccccattagcccaatagataccgtaccagtcaagcttaagcctggtatggacggtccgcgagttaagcaatggccacttactgaggagaaaatcaaggcactcatcgagatctgcaccgaaatggagaaggagggcaaaataagcaggattggtcccgagaatccatataatacgccgatcttcgcgataaaaaagaaggacggcaccaaatggcgaaaactggttgacttccgggagcttaacaaaaaaactcaggatttttgggaagttcaactcgggatcccacacccgtctggtcttaaaaaaaaaaaaagcgtaacagtccttgacatcggcgatgcctactttagcgtgcctctcgataaggagttcagaaaatacacggctttcactgtaccaagcacaaacaatgagactcctggggtcagataccagtacaatgtccttcccatggggtggaaaggaagccccgcaatattccagtgctcaatgacg

SEQ ID NO: 220 - protein, 501 aa, synthetic construct
  REGION 1..501: note = Description of Artificial Sequence: Synthetic polypeptide
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQPPVVAKEIVASCDKCQLKGEAMHGQVDCSPGIWQLDCTHLEGKIILVAVHVASGYIEAEVIPAETGQETAYFLLKLAGRWPVKTMAARASVLSGGELDRWEKIRLRPGGKKKYRLKHIVWASRELERFAVNPGLLETAATVKAACWWAGIKQEFGIPYNPQSQGVVESMNKELKKIIGQVRDQAEHLKTAVQMAVFIHNFKRKGGIGGYSAGERIVDIIATQGYFPDWQNYTPGPGTRYPLTFGWCFKLVPVKEKVYLAWVPAHKGIGGNEQVDKLVS

SEQ ID NO: 221 - protein, 501 aa, synthetic construct
  REGION 1..501: note = Description of Artificial Sequence: Synthetic polypeptide
FPQITLWQRPLVTIKIGGQLKEALLDTGADDTVLEEMNLPGRWKPKMIGGIGGFIKVRQYDQWGFTTPDKKHQKEPPFLWMGYELHPDKWTVQPIVLPEKDSWTVNDIQKLVGKLNWASQIYPGIKVAAQEEEEVGFPVKPQVPLRPMTFKGALDLSHFLREKGGLEGLIITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 222 - protein, 1030 aa, synthetic construct
  REGION 1..1030: note = Description of Artificial Sequence: Synthetic polypeptide
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQPPVVAKEIVASCDKCQLKGEAMHGQVDCSPGIWQLDCTHLEGKIILVAVHVASGYIEAEVIPAETGQETAYFLLKLAGRWPVKTMAARASVLSGGELDRWEKIRLRPGGKKKYRLKHIVWASRELERFAVNPGLLETAATVKAACWWAGIKQEFGIPYNPQSQGVVESMNKELKKIIGQVRDQAEHLKTAVQMAVFIHNFKRKGGIGGYSAGERIVDIIATQGYFPDWQNYTPGPGTRYPLTFGWCFKLVPVKEKVYLAWVPAHKGIGGNEQVDKLVSRAKRAPVKQTLNFDLLKLAGDVESNPGPFPQITLWQRPLVTIKIGGQLKEALLDTGADDTVLEEMNLPGRWKPKMIGGIGGFIKVRQYDQWGFTTPDKKHQKEPPFLWMGYELHPDKWTVQPIVLPEKDSWTVNDIQKLVGKLNWASQIYPGIKVAAQEEEEVGFPVKPQVPLRPMTFKGALDLSHFLREKGGLEGLIITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 223 - protein, 1028 aa, synthetic construct
  REGION 1..1028: note = Description of Artificial Sequence: Synthetic polypeptide
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVSRAKRAPVKQTLNFDLLKLAGDVESNPGPLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 224 - protein, 1021 aa, synthetic construct
  REGION 1..1021: note = Description of Artificial Sequence: Synthetic polypeptide
TVKAACWWAGIKQEFGIPYNPQSQGVVESMNKELKKIIGQVRDQAEHLKTAVQMAVFIHNFKRKGGIGGYSAGERIVDIIAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQMAARASVLSGGELDRWEKIRLRPGGKKKYRLKHIVWASRELERFAVNPGLLETPPVVAKEIVASCDKCQLKGEAMHGQVDCSPGIWQLDCTHLEGKIILVAVHVASGYIEAEVIPAETGQETAYFLLKLAGRWPVKTKEKVYLAWVPAHKGIGGNEQVDKLVSTQGYFPDWQNYTPGPGTRYPLTFGWCFKLVPVRAKRAPVKQTLNFDLLKLAGDVESNPGPFPQITLWQRPLVTIKIGGQLKEALLDTGADDTVLEEMNLPGRWKPKMIGGIGGFIKVRQYDQEEVGFPVKPQVPLRPMTFKGALDLSHFLREKGGLEGLIPKFKLPIQKETWETWWTEYWQATWIPEWEFVNTPPLVKLWYQLEKEPIVGAETFYVDGAANRETKWGFTTPDKKHQKEPPFLWMGYELHPDKWTVQPIVLPEKDSWTVNDIQKLVGKLNWASQIYPGIKVITKIQNFRVYYRDSRDPLWKGPAKLLWKGEGAVVIQDNSDIKVVPRRKAKIIRDYGKQMAGDDCVASRQDEDGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 225 - DNA, 3090 bp, synthetic construct
  misc_feature 1..3090: note = Description of Artificial Sequence: Synthetic polynucleotide
  source 1..3090: mol_type = other DNA
atttctcctcggacgctgaatgcatgggtgaaggtagtggaggaaaaggcattttcaccagaagtcattccgatgttctccgcgctctccgagggtgctacgccacaggacttgaatacgatgctgaataccgttggtggccatcaggcggcgatgcagatgttgaaggagacaattaacgaagaagccgccgaatgggacagattgcacccggtgcatgcggggccaatagctcctggccagatgcgcgagcctaggggttctgacattgctggtacaacaagtacccttcaggagcagattggttggatgacgaataaccctcccatacctgttggcgagatctataagcgctggattatacttgggctgaataagatagtccgaatgtattctcccacctctattctggatattcggcaaggacctaaggagccgtttagagactacgtagaccggttttacaaaaccctgcgggcggaacaagcttctcaggaagtcaaaaattggatgactgagaccttgctcgtccagaatgcgaacccggactgtaaaacaatactcaaagcgctgggccccgctgcaaccctggaagaaatgatgacggcttgtcagggagtaggaggccccggacataaggcacgagtgttggcagaagccatgagccagccgcctgtcgtcgcgaaagaaatcgtcgcttcttgcgacaaatgtcagctgaagggggaggcgatgcacggtcaagttgattgctctcccggtatttggcaattggactgtacccaccttgaaggcaaaattattctggttgcagtgcacgtagcatccggttacatcgaagctgaagtgatacccgcagagacaggccaggagacggcttatttcctccttaagcttgcgggtcggtggcccgtaaagaccatggctgctcgggcatctgtcctctccggtggtgaactcgaccgatgggaaaagattcgattgcgccccggaggaaagaagaaatataggctgaaacatattgtgtgggcatcacgggaacttgagcgatttgcggtaaacccaggccttttggaaacagctgcaactgtgaaagcggcttgctggtgggcggggataaaacaggagtttggtatcccgtacaatccccaatctcagggggtagtagaaagcatgaacaaagaattgaaaaaaataattggccaggttcgcgaccaagccgagcacctcaagaccgctgtacagatggctgtatttattcacaacttcaagcggaagggcggaataggaggatatagcgcaggggaaaggattgttgatattattgcaacacaaggttactttcctgactggcaaaactacacaccgggccctggcacgcgctatccccttacgttcggttggtgcttcaagctggtgccggtaaaagaaaaagtttatttggcatgggttcctgcacataaaggaatagggggtaacgaacaagttgacaaactcgtcagccgcgctaaaagagccccagtcaagcagaccctgaattttgacctgcttaaattggctggggacgtcgagagtaacccgggacccttcccacaaattacactctggcagcgaccactggtaacaatcaaaatagggggacaattgaaagaagcactcctggatacgggcgcggacgatacagtcctggaggaaatgaatctccccggccgctggaaacctaagatgataggggggatcggtggatttattaaagtgcggcagtacgatcaatggggttttacgacacctgacaagaaacatcaaaaggagccgccatttctttggatgggttatgagcttcatccggataaatggactgttcagccgattgtcctccccgagaaggatagttggactgtgaacgacatccagaagctggtcgggaaacttaattgggccagtcaaatatatccaggtattaaagttgccgcacaagaagaggaggaagtagggttccccgtgaaaccgcaagtccctctccggcccatgacctttaagggcgctctcgacctgtcccatttcctccgcgaaaaggggggtttggaaggcttgatcattaccaagattcaaaacttcagggtctattatcgagacagtcgcgatcccctttggaaaggacctgcgaaacttctttggaaaggagaaggagccgtggtaattcaggataattctgacataaaggtcgtcccacgccgaaaagcgaagattataagagattatggcaagcagatggccggggatgattgtgtcgcaagtagacaggatgaagaccctaaattcaagcttccaatccagaaggaaacgtgggagacatggtggaccgagtattggcaagctacttggatcccagaatgggaatttgtgaacactcccccactcgtaaagctgtggtaccagcttgaaaaagaacctatagtcggggcggagacgttctatgtagacggcgccgctaatcgagagacaaaagcagcaggcacggtactggtaggcccgacccctgtcaacatcattggacgaaatctgttgacccagattgggtgtacccttaactttcccatttcaccaatagagaccgtcccggttaaactgaaaccgggtatggatggtcccaaagtaaaacagtggccacttaccgaggagaagattaaggcactcgttgaaatatgtacagaaatggaaaaagaggggaaaatctctaaaattggccctgaaaatccgtacaacactccggtattcgccataaaaaagaaggactctaccaagtggcgcaaactcgtggactttagagaactgaataaaaggacccaggacttttgggaagtccagctgggtattcctcaccccgctggtctcaagaaaaagaaaagtgtcactgtcttggatgttggagatgcgtacttttcagtacctcttgataaagattttcgaaagtataccgcgtttaccattccctccataaataacgaaacaccggggatcaggtatcaatataacgtgcttccacaaggctggaagggttcaccggctattttccaatcttctatgacg

SEQ ID NO: 226 - DNA, 3090 bp, synthetic construct
  misc_feature 1..3090: note = Description of Artificial Sequence: Synthetic polynucleotide
  source 1..3090: mol_type = other DNA
atgctttcaccgagaaccttgaacgcgtgggttaaagtaatcgaagagaaggcatttagcccggaggtaatccccatgtttaccgcactttccgaaggtgctacaccccacgacctgaatacaatgttgaatactattggcggccaccaagctgccatgcagatgcttaaggacactattaacgaggaagccgccgaatgggatcgcgttcaccctgttcacgcgggtccagtcgcaccaggacaaatgcgagaccctcgggggtctgacatcgcaggatcaactagtacattgcaggaacagattgcttggatgaccaataaccctccgatcccggtaggtgacatttataaacggtggatcatcatgggattgaacaaaatagtccgaatgtacagtccagtgagtattcttgacattaaacagggaccaaaggagccgttccgggactacgttgaccggttttacaggaccctgagagccgaacaggcttcccaggatgtgaaaaactggatgacagagaccctgcttgttcagaattcaaatcctgactgtaaaacgattctgaaggcactcggtcccggcgccacgctggaggaaatgatgtcagcttgtcagggagtaggaggaccttctcataaggcacgcgtccttgcagaggccatgtgccaagtcgcgaaagagatcgtggcctgttgtgacaagtgccaacttaagggagaagccatccatgggcaggtcgattgttctccgggcgtttggcaattggactgcacgcacttggagggtaaggtgattcttgttgccgttcatgtcgcaagcggttacattgaagcggagattatcccaactgagaccggccaagagactgcatatttcatccttaaactggcgggaagatggccggtcacgactatggctgcacgagcatccatactgtctgggggaaagctcgacaagtgggagaagattcgacttaggcctggaggaagaaaaaagtataagttgaaacatcttgtttgggcatcacgagaacttgaacggtttgctctgaatccgggattgcttgaaaccgccgcggctgtaaaggcagcatgttggtgggctggtgtcaaacaagagttcggtattccctataacacccaaagtcaaggcgtcgtagaatccatgaacaatgaacttaagaaaattatcggacaaatccgggaccaagcggaacacctcaaaactgctgtccagatggcagttctgattcataactttaaaagaaagggggggataggggagtattctgcaggtgaacgaattatagacataattgccacacaagggttttttccagattggcaaaattatacaccgggccctgggataagattcccgctcaccttcggctggtgcttcaaactggtacccttgctgatcaagaaagagaagatctatcttgcttgggtgccagcccataaagggatcgggggtaacgaacaaatcgataagctggtgtctagagctaaacgggctcccgtaaaacagaccttgaacttcgatctgcttaaattggcaggggacgtggaaagcaaccccgggccaatgctgccccagataacactttggcaacgccccatcgtgacaatcaagatcggtggccaaattaaggaagcactcttggacacgggagcagacgacactgtgctggaggatatgaacctgccgggcaagtggaaaccaaagatgatcgggggcattggcgggttcataaaggttaaacagtacgaccaatgggggttgacaacgcctgataagaagcatcaaaaagatcccccatttttgtggatgggttatgaacttcacccggacaggtggaccgttcagccgatagagctcccagaaaaggagtcttggacagttaatgacatacagaaacttattggcaaacttaactgggcttcacagatttatgccggcatcaaagtcgccgcccaggaagaagaagaggtaggttttcccgtacgacctcaggttcctcttcggcctatgacctataagggtgcgcttgatctttctcacttccttaaagaaaaaggaggtctggaaggtatcacgaaacttcagaattttcgggtgtattaccgggacaacagagacccgctttggaaggggccggctaggcttctgtggaaaggcgagggagcggtagttatccaggataactctgagataaaggtagtaccccgacggaaggtaaagatcatcagagactacggcaagaggatggctggagacgactgtgtggccgggcgacaggatgaagatcctaaattcaggctgccaatccaaaaagagacgtgggacacatggtggaccgattattggcaggctacgtggatccccgaatgggagtttaccaatactccgccactcgtgaagctttggtaccaattggagacagagcctatagccggcgttgagaccttctacgtggatggggccagcaacagagaaaccaaagcggccggaacggtcctgatcggtcccacacctgttaacatcatagggcgcaatctgcttacgcaattggggtgcacattgaattttccaatatcccctattgataccgtgccggttaaattgaagccgggtatggacgggcctcgggtcaagcagtggcccctgaccgaagaaaagatcaaagccctgattgagatctgcacggaaatggaaaaagagggcaagattagtcgcatcggcccggagaacccatacaatactcctatttttgcaattaaaaaaaaggacggaacaaagtggaggaaacttgtagatttcagagagcttaataagaaaactcaggacttctgggaggtccaactcggtattccgcatccctccggacttaagaagaaaaagtcagtaaccgtcttggatataggggacgcttatttttcagtgcccctcgataaagaatttcgcaaatacacggcgtttactgtgccatctactaataacgaaacgccaggcgtgagatatcaatacaacgtccttcctatgggctggaagggttcacccgcaatttttcagtgctccatgacc

SEQ ID NO: 227 - protein, 1029 aa, synthetic construct
  REGION 1..1029: note = source = /note="Description of Artificial Sequence: Synthetic polypeptide"
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVSRAKRAPVKQTLNFDLLKLAGDVESNPGPMLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 228 - protein, 507 aa, unidentified
MGARNSVLSGKKADELEKIRLRPNGKKKYMLKHVVWAANELDRFGLAESLLDNKEGCQKILSVLAPLVPTGSENLKSLYNTVCVIWCIHAEEKVKHTEEAKQIVQRHLVVETGTADKMPATSRPTAPPSGRGGNYPVQQVGGNYVHLPLSPRTLNAWVKLVEEKKFGAEVVPGFQALSEGCTPYDINQMLNCVGEHQAAMQIIREIINEEAADWDLQHPQPGPLPAGQLREPRGSDIAGTTSTVEEQIQWMYRQQNPIPVGNIYRRWIQLGLQKCVRMYNPTNILDVKQGPKEPFQSYVDRFYKSLRAEQTDPAVKNWMTQTLLIQNANPDCKLVLKGLGMNPTLEEMLTACQGIGGPGQKARLMAEALKEALRPDQLPFAAVQQKGQRRTIKCWNCGKEGHSARQCRAPRRQGCWGCGKTGHVMAKCPERQAGFLGFGPWGKKPRNFPMAQMPQGLTPTAPPEDPAVDLLKNYMKMGRKQRENRERPYKEVTEDLLHLNSLFGEDQ

SEQ ID NO: 229 - protein, 531 aa, unidentified
PQFSLWRRPVVTAYIEEQPVEVLLADDSIVTGIELGPNYTPKIVGGIGGFINTKEYKDVKIKVLGKVIKGTIMTGDTPINIFGRNLLTAMGMSLNFPIAKVEPIKVTLKPGKEGPKLRQWPLSKEKIIALREICEKMEKDGQLEEAPPTNPYNTPTFAIKKKDKNKWRMLIDFRELNKVTQDFTEVQLGIPHPAGLAKRRRITVLDVGDAYFSIPLDEEFRQYTAFTLPSVNNAEPGKRYIYKVLPQGWKGSPAIFQYTMRNVLEPFRKANPDVTLIQILIASDRTDLEHDRVVLQLKELLNGIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPIEATVIKSQDNQWSYKIHQEDKVLKVGKFAKVKNTHTNGVRLLAHVVQKIGKEALVIWGEVPKFHLPVEREIWEQWWTDYWQVTWIPDWDFVSTPPLVRLVFNLVKEPIQGAETF

SEQ ID NO: 230 - protein, 409 aa, unidentified
YVDGSCNRQSREGKAGYVTDRGRDKAKLLEQTTNQQALEAFYLALADSGPKANIIVDSQYVMGIVAGQPTESESRLVNQIIEEMIKKEAIYVAWVPAHKGIGGNQEVDHLVSQGIRQVLFLEKIEPAQEEHEKYHSNVKELVFKFGIPRLVAKQIVDTCDRCHQKGEAIHGQVNAELGTWQMCTHLEGKIIIVAVHVASGFIEAEVIPQETGRQTALFLLKLAGRWPITHLHTNGANFTSQEVKMVAWWAGIEQTFGVPYNPQSQGVVAMNHHLKTQIDRIREQANSVETIVLMAVHCMNFKRRGGIGDMTPAERLVNMITTEQEIQFQQSKNSKFKNFRVYYREGRDQLWRGPGELLWKGEGAVILKVGTEIKVVPRRKAKIIKDYGGGKELDSGSHLEDTGEAREVA

SEQ ID NO: 231 - protein, 650 aa, unidentified
MGCLGNQLLIALLLVSVLEICCVQYVTVFYGVPAWKNATIPLFCATRNRDTWGTTQCLPDNDDYSELAVNITEAFDAWNNTVTEQAIEDVWNLFETSIKPCVKLTPLCIAMRCNKTETDRWGLTGRAETTTTAKSTTSTTTTTVTPKVINEGDSCIKNNSCAGLEQEPMIGCKFNMTGLKRDKKIEYNETWYSRDLICEQPANGSESKCYMQHCNTSVIQESCDKHYWDAIRFRYCAPPGYALLRCNDSNYSGFAPKCSKVVVSSCTRMMETQTSTWFGFNGTRAENRTYIYWHGNSNRTIISLNKYYNLTMKCRRPGNKTVLPVTIMSGLVFHSQPINERPKQAWCRFGGNWSEAIQEVKETLVKHPRYTGTNDTRKINLTAPAGGDPEVTFMWTNCRGEFLYCKMNWFLNWVEDRDQNSNRWKQQKKPEQQKRNYVPCHIRQIINTWHKVGKNVYLPPREGDLTCNSTVTSLIAEIDWINNNETNITMSAEVAELYRLELGDYKLVEITPIGLAPTDVRRSLTLSAQSRTLLAGIVQQQQQLLDVVKRQHELLRLTVWGTKNLQTRVTAIEKYLKDQAQLNDSLVPNWDNMTWQEWEGKVDFLEANITQLLEEAQIQQEKNMYELQKLNSWDIFGNWFDLTSWIRYIQ